SEQUENCE LISTING (38 sequences)

SEQ ID NO: 1 - protein, 21 aa, ARTIFICIAL
  human insulin A-chain
GIVEQCCTSICSLYQLENYCN

SEQ ID NO: 2 - protein, 30 aa, ARTIFICIAL
  human insulin B-chain
FVNQHLCGSHLVEALYLVCGERGFFYTPKT

SEQ ID NO: 3 - protein, 21 aa, ARTIFICIAL
  A-chain of human insulin analogue
GIVEQCCTSICSLYQLENYCQ

SEQ ID NO: 4 - protein, 21 aa, ARTIFICIAL
  A-chain of human insulin analogue
GIVEQCCTSICSLEQLENYCN

SEQ ID NO: 5 - protein, 21 aa, ARTIFICIAL
  A-chain of human insulin analogue
GIVEQCCTSICSLEQLENYCN

SEQ ID NO: 6 - protein, 22 aa, ARTIFICIAL
  A-chain of human insulin analogue
GIVEQCCTSICSLYQLENYCNK

SEQ ID NO: 7 - protein, 23 aa, ARTIFICIAL
  A-chain of human insulin analogue
KPGIVEQCCTSICSLYQLENYCN

SEQ ID NO: 8 - protein, 31 aa, ARTIFICIAL
  A-chain of human insulin analogue with peptide spacer
GIVEQCCTSICSLYQLENYCQGESGESGESK

SEQ ID NO: 9 - protein, 40 aa, ARTIFICIAL
  A-chain of human insulin analogue with peptide spacer
GIVEQCCTSICSLYQLENYCQGESGESGESGESGESGESK

SEQ ID NO: 10 - protein, 58 aa, ARTIFICIAL
  A-chain of human insulin analogue with peptide spacer
GIVEQCCTSICSLYQLENYCQGESGESGESGESGESGESGESGESGESGESGESGESK

SEQ ID NO: 11 - protein, 29 aa, ARTIFICIAL
  B-chain of human insulin analogue
FVNQHLCGSHLVEALYLVCGERGFFYTPK

SEQ ID NO: 12 - protein, 29 aa, ARTIFICIAL
  B-chain of human insulin analogue
FVNQHLCGSHLVEALYLVCGERGFHYTPK

SEQ ID NO: 13 - protein, 28 aa, ARTIFICIAL
  B-chain of human insulin analogue
KPNQHLCGSHLVEALYLVCGERGFHYPK

SEQ ID NO: 14 - protein, 28 aa, ARTIFICIAL
  B-chain of human insulin analogue
FVNQHLCGSHLVEALYLVCGERGFHYPK

SEQ ID NO: 15 - protein, 29 aa, ARTIFICIAL
  B-chain of human insulin analogue
FVNQHLCGSHLVEALYLVCGERGFHYPGK

SEQ ID NO: 16 - protein, 27 aa, ARTIFICIAL
  B-chain of human insulin analogue
NKPLCGSHLVEALYLVCGERGFFYTPK

SEQ ID NO: 17 - protein, 27 aa, ARTIFICIAL
  B-chain of human insulin analogue
GKPLCGSHLVEALYLVCGERGFFYTPK

SEQ ID NO: 18 - protein, 30 aa, ARTIFICIAL
  B-chain of human insulin analogue
GKPNQHLCGSHLVEALYLVCGERGFFYTPK

SEQ ID NO: 19 - protein, 29 aa, ARTIFICIAL
  B-chain of human insulin analogue
FVNQHLCGSHLVEALYLVCGERGFFYTPR

SEQ ID NO: 20 - protein, 29 aa, ARTIFICIAL
  B-chain of human insulin analogue
FVNQHLCGSHLVEALYLVCGEKGFFYTPR

SEQ ID NO: 21 - protein, 46 aa, ARTIFICIAL
  B-chain of human insulin analogue with peptide spacer
KPGGGGSGGGGSGGGGSFVNQHLCGSHLVEALYLVCGERGFFYTPK

SEQ ID NO: 22 - protein, 46 aa, ARTIFICIAL
  B-chain of human insulin analogue with peptide spacer
KPGGGGSGGGGSGGGGSFVNQHLCGSHLVEALYLVCGERGFHYTPK

SEQ ID NO: 23 - protein, 47 aa, ARTIFICIAL
  B-chain of human insulin analogue with peptide spacer
GKPGGGGSGGGGSGGGGSFVNQHLCGSHLVEALYLVCGERGFFYTPK

SEQ ID NO: 24 - protein, 42 aa, ARTIFICIAL
  B-chain of human insulin analogue with peptide spacer
GKPGGGGSGGGGSFVNQHLCGSHLVEALYLVCGERGFFYTPK

SEQ ID NO: 25 - protein, 37 aa, ARTIFICIAL
  B-chain of human insulin analogue with peptide spacer
GKPGGGGSFVNQHLCGSHLVEALYLVCGERGFFYTPK

SEQ ID NO: 26 - protein, 33 aa, ARTIFICIAL
  B-chain of human insulin analogue with peptide spacer
GKPGFVNQHLCGSHLVEALYLVCGERGFFYTPK

SEQ ID NO: 27 - protein, 49 aa, ARTIFICIAL
  B-chain of human insulin analogue with peptide spacer
GKPRGFFYTPGGGGSGGGGSFVNQHLCGSHLVEALYLVCGERGFFYTPK

SEQ ID NO: 28 - protein, 53 aa, ARTIFICIAL
  B-chain of human insulin analogue with peptide spacer
TYFFGRKPDGGGGSGGGGSGGGGSFVNQHLCGSHLVEALYLVCGERGFFYTPK

SEQ ID NO: 29 - protein, 10 aa, ARTIFICIAL
  peptide spacer
GESGESGESK

SEQ ID NO: 30 - protein, 19 aa, ARTIFICIAL
  peptide spacer
GESGESGESGESGESGESK

SEQ ID NO: 31 - protein, 37 aa, ARTIFICIAL
  peptide spacer
GESGESGESGESGESGESGESGESGESGESGESGESK

SEQ ID NO: 32 - protein, 4 aa, ARTIFICIAL
  peptide spacer
GKPG

SEQ ID NO: 33 - protein, 8 aa, ARTIFICIAL
  peptide spacer
GKPGGGGS

SEQ ID NO: 34 - protein, 13 aa, ARTIFICIAL
  peptide spacer
GKPGGGGSGGGGS

SEQ ID NO: 35 - protein, 18 aa, ARTIFICIAL
  peptide spacer
GKPGGGGSGGGGSGGGGS

SEQ ID NO: 36 - protein, 17 aa, ARTIFICIAL
  peptide spacer
KPGGGGSGGGGSGGGGS

SEQ ID NO: 37 - protein, 20 aa, ARTIFICIAL
  peptide spacer
GKPRGFFYTPGGGGSGGGGS

SEQ ID NO: 38 - protein, 24 aa, ARTIFICIAL
  peptide spacer
TYFFGRKPDGGGGSGGGGSGGGGS

(19) United States
(12) Patent Application Publication
(10) Pub. No.: US 2022/0184184 A1
(43) Pub. Date: Jun. 16, 2022

(54) GLUCOSE SENSITIVE INSULIN DERIVATIVES

(71) Applicant: Novo Nordisk A/S, Bagsvaerd (DK)

(72) Inventors: Thomas Hoeg-Jensen, Broenshoej (DK); Carsten Behrens, Koebenhavn N (DK); Emiliano Clo, Copenhagen (DK); Martin Werner Borchsenius Muenzel, Broenshoej (DK); Per Sauerberg, Farum (DK); Thomas Kruse, Herlev (DK); Jane Spetzler, Broenshoej (DK); Ulrich Sensfuss, Vanloese (DK); Claudia Ulrich Hjoerringgaard, Glostrup (DK); Henning Thoegersen, Farum (DK); Vojtech Balsanek, Praha 14 (CZ); Zuzana Drobnakova, Praha 9 (CZ); Ladislav Droz, Praha 8 (CZ); Miroslav Havranek, Strancice - Praha (CZ); Vladislav Kotek, Praha 9 (CZ); Milan Stengl, Klatovy (CZ); Ivan Snajdr, Praha 4 (CZ); Hana Drusanova, Kromeriz (CZ)

(21) Appl. No.: 17/598,010

(22) PCT Filed: Mar. 27, 2020
(86) PCT No.: PCT/EP2020/058641

§ 371 Date: Sep. 24, 2021

(30) Foreign Application Priority Data
Mar. 29, 2019 (EP) 19166131.3
May 15, 2019 (EP) 19174671.8

(51) Int. Cl. A61K 38/28; A61K 47/54; A61K 47/56; A61P 3/10

(52) U.S. Cl. CPC A61K 38/28; A61K 47/542; A61P 3/10; A61K 47/56; A61K 47/545

(57) ABSTRACT

The present invention relates to novel insulin derivatives and their use in the treatment or prevention of medical conditions relating to diabetes. The insulin derivatives are glucose sensitive and display glucose-sensitive albumin binding. The invention also relates to novel intermediates. Finally, the invention provides a pharmaceutical composition comprising the insulin derivatives of the invention and the use of such a composition in the treatment or prevention of medical conditions relating to diabetes.

### TECHNICAL FIELD

The present invention relates to novel insulin derivatives, and their pharmaceutical use. Furthermore, the invention relates to pharmaceutical compositions comprising such insulin derivatives, and to the use of such compounds for the treatment or prevention of medical conditions relating to diabetes.

### BACKGROUND

Insulin is the most effective drug for treatment of high blood glucose, but insulin dosing is a delicate balance between too much and too little since the physiological glucose window is narrow. Healthy persons have glucose levels at fasted state near 5 mM, and diabetes patients try to dose both meal and basal insulin preparations to get near 5 mM. However, blood glucose values below approximately 3 mM (hypoglycemia) often occur during insulin treatments, and hypoglycemia can result in discomfort, loss of conciseness, brain damage or death. Diabetes patients are thus hesitant to treat their high or moderately high blood sugar values aggressively out of fear for hypoglycaemia. It could help diabetes treatment if insulin drugs were developed that were only active or released from a depot at higher blood glucose values and were inactive or weakly active at lower glucose values. Such goals have been approached in numerous papers since the 1970's (Brownlee et al. Science 1979, 1190; Zaykov et al. Nature Rev. Drug Disc. 2016, 425) but most often via glucose-sensitive polymers that entrap and release insulin in a glucose-depending fashion from subcutaneous depots. Such systems are however slow, and thus not good for treatment of quickly fluctuating blood glucose values after for example meals. Consequently, subcutaneous glucose-sensitive release systems have never reached clinical trials.

It would be better if glucose-sensitive tuning of insulin bioactivity could be done in the blood. One approach that could fulfil this wish could be glucose-sensitive albumin binding, as described before with fatty acid-monoboronate insulin derivatives where the fatty acid part gives rise to albumin binding (Novo Nordisk WO2011/000823; WO 2014/093696; Chou et al. Proc. Nat. Acad. Sci. 2015, 2401). The main driving force of the albumin interaction in these systems arise from the fatty acid part of the fatty acid-monoboronate insulin derivative (not the boronate), and the impact of glucose on albumin affinity is weak. To increase the glucose sensitivity of the albumin binding, there is thus a need for glucose sensitive albumin binding motifs that are directly displaced by glucose. Monoboronates are known to bind glucose and other sugars with affinities (Kd) in the medium to high millimolar range (Hansen et al. Sensors Actuators B 2012, 45). However, to provide adequate glucose sensitivity at physiological glucose levels, stronger affinities to glucose are needed. Diboron compounds with two boronates/boroxoles placed in proper geometry relative to the hydroxy groups on glucose can give increased glucose affinity relative to monoborons, namely low mM Kd or sub-mM Kd (Hansen et al. Sensors Actuators B 2012, 45). Most such diborons described in the literature include fluorescent probes, because the aim of those studies were to make optical glucose sensors. Fluorescent probes are not desirable in drug candidates as these probes can be sensitive to light, toxic and coloured. There is thus a need for insulin derivatives with increased glucose sensitivity within physiological blood glucose levels.

### SUMMARY

In the broadest aspect, the present invention relates to insulin derivatives.

The compounds of the present invention have surprisingly been found to bind to both albumin (HSA) and glucose, and the HSA affinity is glucose-sensitive. Human insulin receptor (HIR) affinity in presence of HSA thus also become glucose-sensitive. The fraction of insulin that is HSA-bound is shielded from binding to HIR, but glucose-promoted release from HSA increase the free fraction of insulin, and glucose thus increase the HIR affinity. As opposed to previously disclosed insulin derivatives with alleged glucose-sensitive albumin binding, the compounds of the present invention do not rely on a fatty acid part for the albumin binding, but comprises an albumin binding motifs that are directly displaced by glucose, leading to increased impact of glucose on the albumin binding, and thus increased glucose sensitivity of the insulin.

Albumin binding can in general prolong the in vivo half-life of peptides and protein-based drugs. The prolonged effect is achieved as the albumin bound fraction is protected from enzymatic degradation and kidney elimination, and only the free fraction is biological active, thus preventing receptor mediated clearance of the albumin bound fraction.

The compounds of the present invention thus display insulin activity dependent of the glucose concentration, and thus serves as glucose sensitive insulin derivatives.

In one aspect, the compounds of the present invention comprise insulin or an analogue thereof, and one or more modifying groups.

In one aspect, the modifying group has affinity to glucose and to albumin.

In one aspect, the insulin peptide or analogue thereof optionally comprises a spacer.
In one aspect, the compound of the present invention comprises

i) human insulin or a human insulin analogue; and

ii) one or more modifying groups M, wherein each of the modifying groups M comprises two aryl moieties, wherein a boron atom is attached to each of the two aryl moieties. Each of the one or more modifying groups M is attached, optionally via a spacer, to the amino group of the N-terminal amino acid residue of the A-chain or B-chain of said human insulin or human insulin analogue or to the epsilon amino group of a lysine in said human insulin or human insulin analogue.

In one embodiment, the one or more modifying groups M is attached, optionally via a spacer, to the sulfide of a free cysteine in said human insulin or human insulin analogue.

In one aspect, the compound of the present invention comprises

i) human insulin or a human insulin analogue; and

ii) two or more modifying groups M, wherein each of the modifying groups M comprises two aryl moieties, wherein a boron atom is attached to each of the two aryl moieties. Each of the two or more modifying groups M is attached, optionally via a spacer, to the amino group of the N-terminal amino acid residue of the A-chain or B-chain of said human insulin or human insulin analogue or to the epsilon amino group of a lysine in said human insulin or human insulin analogue.

As can be seen from the examples, compounds having two or more modifying groups M in general display a higher degree of glucose sensitivity (higher glucose factor) than compounds having only one modifying group M.

In one aspect, the invention provides intermediate products in the form of novel insulin analogues, including novel insulin analogues comprising a peptide spacer.

In one aspect, the compounds of the present invention activate the insulin receptor as a function of the glucose concentration in the blood and tissue.

In one aspect, the compounds of the present invention have low availability (low non-bound, plasma free fraction) and thus low or no activity during situations of low blood glucose, for example levels below about 3 mM glucose (hypoglycaemia).

In one aspect, the compounds of the present invention have high availability (high non-bound, plasma free fraction) and thus high activity in response to high blood glucose, for example above about 10 mM glucose (hyperglycaemia).

In one aspect, the compounds of the present invention display glucose-sensitive albumin binding.

In another aspect, the invention relates to a pharmaceutical composition comprising a compound according to the invention. In another aspect, the invention relates to a compound according to the invention for use as a medicament. In another aspect, the invention relates to a compound according to the invention for use in the treatment of diabetes. In another aspect, the invention relates to medical use(s) of the compounds according to the invention. The invention may also solve further problems that will be apparent from the disclosure of the exemplary embodiments.

### BRIEF DESCRIPTION OF DRAWINGS

FIG. 1 shows PK profile of i.v. bolus of insulin aspart at 10 mM and 3.5-4 mM glucose (Example E).

FIG. 2 shows PK profile i.v. bolus of insulin degludec at 10 mM and 3.5-4 mM glucose (Example E).

FIG. 3 shows PK profile of i.v. bolus of example number 210 (triangles) and example number 211 (circles) at 10 mM (closed) and 3.5-4 mM (open) glucose (Example E).

FIG. 4 shows PK profile of i.v. bolus of example number 233 (triangles) and example number 234 (squares) at 10 mM (closed) and 3.5-4 mM (open) glucose (Example E).

FIG. 5 shows PK profile of i.v. bolus of example number 240 (triangles) and example number 227 (circles) at 10 mM (closed) and 3.5-4 mM (open) glucose (Example E).

FIG. 6 shows PK profile of i.v. bolus of example number 241 (triangles) and example number 181 (squares) at 10 mM (closed) and 3.5-4 mM (open) glucose (Example E).

FIG. 7 shows PK profile of i.v. bolus of example number 205 (triangles) and example number 239 (squares) at 10 mM (closed) and 3.5-4 mM (open) glucose (Example E).

FIG. 8 shows PK profile of i.v. bolus of example number 285 (triangles) and example number 273 (squares) at 10 mM (closed) and 3.5-4 mM (open) glucose (Example E).

FIG. 9 shows PK profile of i.v. bolus of example number 280 (triangles) and example number 272 (squares) at 10 mM (closed) and 3.5-4 mM (open) glucose (Example E).

FIG. 10 shows a comparison between the baseline-adjusted glucose infusion rate areas under the curve for clamps at 3.5-4 mM glucose vs 10 mM glucose for example numbers 205, 239, 272 and 280 (Example E).

### DESCRIPTION

The present invention relates to insulin derivatives. In one aspect, the present invention relates to glucose sensitive insulin derivatives.

In one embodiment, the present invention relates to a compound comprising human insulin or an analogue thereof and a modifying group, which modifying group displays affinity to both glucose and to albumin.

In one embodiment, the modifying group displays glucose-sensitive albumin binding.

In one embodiment, the insulin analogue is an analogue of human insulin (SEQ ID NO:1 and SEQ ID NO:2).

In one embodiment, the human insulin or human insulin analogue of the present invention may comprise a spacer.

In one embodiment, the invention provides a compound comprising a human insulin or a human insulin analogue; and one or more modifying groups M, wherein each of the modifying groups M comprises two aryl moieties, wherein a boron atom is attached to each of the two aryl moieties. Each of the one or more modifying groups M is attached, optionally via a spacer, to the amino group of the N-terminal amino acid residue of the A-chain or B-chain of said human insulin or human insulin analogue or to the epsilon amino group of a lysine in said human insulin or human insulin analogue.

In one embodiment, the invention provides a compound comprising a human insulin or a human insulin analogue; and two or more modifying groups M, wherein each of the modifying groups M comprises two aryl moieties, wherein a boron atom is attached to each of the two aryl moieties. Each of the two or more modifying groups M is attached, optionally via a spacer, to the amino group of the N-terminal amino acid residue of the A-chain or B-chain of said human insulin or human insulin analogue or to the epsilon amino group of a lysine in said human insulin or human insulin analogue. The modifying groups M may also be attached, optionally via a spacer, to the sulfide of a free cysteine in said human insulin or human insulin analogue.

### General Definitions

The term “compound” is used herein to refer to a molecular entity, and “compounds” may thus have different structural elements besides the minimum element defined for each compound or group of compounds. The term “compound” is also meant to cover pharmaceutically relevant forms hereof, i.e. the invention relates to a compound as defined herein or a pharmaceutically acceptable salt, amide, or ester thereof.

The term “peptide” or “polypeptide”, as e.g. used in the context of the invention, refers to a compound which comprises a series of amino acids interconnected by amide (or peptide) bonds. In a particular embodiment the peptide consists of amino acids interconnected by peptide bonds.

The term “analogue” generally refers to a peptide, the sequence of which has one or more amino acid changes when compared to a reference amino acid sequence. Analogues “comprising” certain specified changes may comprise further changes, when compared to their reference sequence. In particular embodiments, an analogue “has” or “comprises” specified changes. In other particular embodiments, an analogue “consists of” the changes. When the term “consists” or “consisting” is used in relation to an analogue e.g. an analogue consists or consisting of a group of specified amino acid mutations, it should be understood that the specified amino acid mutations are the only amino acid mutations in the analogue. In contrast an analogue “comprising” a group of specified amino acid mutations may have additional mutations.

The term “derivative” generally refers to a compound which may be prepared from a native peptide or an analogue thereof by chemical modification, in particular by covalent attachment of one or more substituents.

In the context of the present invention, the modifying group M is a covalently attached substituent.

The term “amino acid” includes proteinogenic (or natural) amino acids (amongst those the 20 standard amino acids), as well as non-proteinogenic (or non-natural) amino acids. Proteinogenic amino acids are those which are naturally incorporated into proteins. The standard amino acids are those encoded by the genetic code. Non-proteinogenic amino acids are either not found in proteins, or not produced by standard cellular machinery (e.g., they may have been subject to post-translational modification).

In general, amino acid residues (peptide/protein sequences) may be identified by their full name, their one-letter code, and/or their three-letter code. These three ways are fully equivalent. In what follows, each amino acid of the peptides of the invention for which the optical isomer is not stated is to be understood to mean the L-isomer (unless otherwise specified). Amino acids are molecules containing an amino group and a carboxylic acid group, and, optionally, one or more additional groups, often referred to as a side chain. Herein, the term “amino acid residue” is an amino acid from which, formally, a hydroxy group has been removed from a carboxy group and/or from which, formally, a hydrogen atom has been removed from an amino group.

As is apparent from the below examples, amino acid residues may be identified by their full name, their one-letter code, and/or their three-letter code. These three ways are fully equivalent and interchangeable.

Herein, the term “aryl” means a cyclic or polycyclic aromatic ring having from 5 to 12 carbon atoms. The term aryl includes both monovalent, divalent, and multivalent species. Examples of aryl groups include, but are not limited to, phenyl, biphenyl, naphthyl, anthracenyl and the like. In a particular embodiment, an aryl is phenyl. Herein, the term “aryl” also comprises a “heteroaryl”. The term “heteroaryl” means an aromatic mono-, bi-, or polycyclic ring incorporating one or more (for example 1-4, particularly 1, 2 or 3) heteroatoms selected from nitrogen, oxygen or sulfur.

Insulin

The term “human insulin” as used herein means the human insulin hormone whose structure and properties are well-known. Human insulin has two polypeptide chains, named the A-chain and the B-chain. The A-chain is a 21 amino acid peptide and the B-chain is a 30 amino acid peptide, the two chains being connected by disulphide bridges: a first bridge between the cysteine in position 7 of the A-chain and the cysteine in position 7 of the B-chain, and a second bridge between the cysteine in position 20 of the A-chain and the cysteine in position 19 of the B-chain. A third bridge is present between the cysteines in position 6 and 11 of the A-chain.

The human insulin A-chain has the following sequence: GIVEQCCTSICSLYQLENYCN (SEQ ID NO:1), while the B-chain has the following sequence: FVNQHLCGSHLVEALYLVCGERGFFYTPKT (SEQ ID NO:2).

The term “insulin peptide”, “insulin compound” or “insulin” as used herein means a peptide which is either human insulin or an analogue or a derivative thereof with insulin activity, i.e., which activates the insulin receptor.

Insulin Analogues

The term “insulin analogue” as used herein means a modified human insulin wherein one or more amino acid residues of the insulin have been substituted by other amino acid residues and/or wherein one or more amino acid residues have been deleted from the insulin and/or wherein one or more amino acid residues have been added and/or inserted to the insulin.

The term “insulin analogue” as used herein means an insulin analogue displaying insulin activity, i.e. which activates the insulin receptor.

The insulin analogue comprises less than 10 amino acid modifications (substitutions, deletions, additions (i.e. extensions), insertions, and any combination thereof) relative to human insulin, alternatively less than 9, 8, 7, 6, 5, 4, 3, 2 or 1 modification relative to human insulin. In one aspect, the insulin analogue has less than 10 amino acid modifications (substitutions, deletions, additions (i.e. extensions), insertions, and any combination thereof) relative to human insulin, alternatively less than 9, 8, 7, 6, 5, 4, 3, 2 or 1 modification relative to human insulin.

Modifications in the insulin molecule are denoted stating the chain (A or B), the position, and the one or three letter code for the amino acid residue substituting the native amino acid residue.

Herein terms like “A1”, “A2” and “A3” etc. indicates the amino acid in position 1, 2 and 3 etc., respectively, in the A chain of insulin (counted from the N-terminal end). Similarly, terms like B1, B2 and B3 etc. indicates the amino acid in position 1, 2 and 3 etc., respectively, in the B chain of insulin (counted from the N-terminal end). Using the one letter codes for amino acids, terms like A21A, A21G and A21Q designates that the amino acid in the A21 position is A, G and Q, respectively. Using the three letter codes for amino acids, the corresponding expressions are A21Ala, A21Gly and A21Gln, respectively.

By “desB30” is meant a natural insulin B chain or an analogue thereof lacking the B30 amino acid.

Herein the terms “A-1” or “B-1” indicate the positions of the amino acids N-terminally to A1 or B1, respectively. The terms A-2 or B-2 indicate the positions of the first amino acids N-terminally to A-1 or B-1, respectively.

The terms “A22” or “B31” indicate the positions of the amino acids C-terminally to A21 or B30, respectively.

Thus, e.g., A14E B1K B2P B25H desB27 desB30 human insulin is an analogue of human insulin where the amino acid in position 14 in the A chain is substituted with glutamic acid, the amino acid in position 1 in the B chain is substituted with lysine, the amino acid in position 2 in the B chain is substituted with proline, the amino acid in position 25 in the B chain is substituted with histidine, and the amino acids in positions 27 and 30 in the B chain are deleted.

Examples of insulin analogues having substitutions are such wherein Tyr at position A14 is substituted with Glu. Furthermore, the amino acid in position B1 or B4 may be substituted with Lys. The amino acid in position B2 may be substituted with Pro. The amino acid in position B25 may be substituted with His.

Examples of insulin analogues with deletions are analogues where the B30 amino acid in human insulin has been deleted (desB30 human insulin), insulin analogues wherein the B1 amino acid in human insulin has been deleted (desB1 human insulin), insulin analogues wherein the B1 and B2 amino acids in human insulin has been deleted (desB1 desB2 human insulin), and desB27 human insulin.

Examples of insulin analogues wherein the A-chain and/or the B-chain have an N-terminal extension (i.e. where one or more amino acid residues have been added to the N-terminus) is a human insulin analogue comprising A-2K and A-1P, i.e. an analogue of human insulin, wherein the A-chain has been extended at the N-terminal with KP. Another example is a human insulin analogue where one glycine residue is added to the N-terminal of the B-chain, i.e. the human insulin analogue comprises B-1G.

Examples of insulin analogues wherein the A-chain and/or the B-chain have a C-terminal extension (i.e. where one or more amino acid residues have been added to the C-terminus) are human insulin analogues comprising A22K.

Further examples are insulin analogues comprising combinations of the mentioned mutations.

Examples of insulin analogues include:

desB30 human insulin (SEQ ID NO:1 and SEQ ID NO:11);

A21Q desB30 human insulin (SEQ ID NO:3 and SEQ ID NO:11);

A14E B25H desB30 human insulin (SEQ ID NO:4 and SEQ ID NO:12);

A14E B1K B2P B25H desB27 desB30 human insulin (SEQ ID NO:4 and SEQ ID NO:13);

A14E A22K B25H desB27 desB30 human insulin (SEQ ID NO:5 and SEQ ID NO:14);

A14E A22K B25H B27P B28G desB30 human insulin (SEQ ID NO:5 and SEQ ID NO:15);

A14E desB1-B2 B4K B5P desB30 human insulin (SEQ ID NO:4 and SEQ ID NO:16);

A14E desB1-B2 B3G B4K B5P desB30 human insulin (SEQ ID NO:4 and SEQ ID NO:17);

A14E B-1G B1K B2P desB30 human insulin (SEQ ID NO:4 and SEQ ID NO:18);

A22K desB30 human insulin (SEQ ID NO:6 and SEQ ID NO:11);

A22K B29R desB30 human insulin (SEQ ID NO:6 and SEQ ID NO:19);

A22K B22K B29R desB30 human insulin (SEQ ID NO:6 and SEQ ID NO:20); and

A-2K A-1P desB30 human insulin (SEQ ID NO:7 and SEQ ID NO:11).

Spacer

As stated above, the insulin analogue of the invention comprises less than 10 amino acid modifications (substitutions, deletions, extensions, and any combination thereof) relative to human insulin, alternatively less than 9, 8, 7, 6, 5, 4, 3, 2 or 1 modification relative to human insulin. In addition to these up to 9 modifications, the human insulin or human insulin analogue of the present invention may comprise a spacer at the C-terminal of the A-chain of human insulin or the human insulin analogue, or at the N-terminal of the B-chain of human insulin or the human insulin analogue.

In one embodiment, the spacer is a peptide, which is herein referred to as a spacer peptide or a peptide spacer. In another embodiment, the spacer is a non-peptide linker L.

Peptide Spacer

Various spacer peptides are known in the art, and may be used in the compounds of the present invention. In one embodiment, the spacer is a peptide segment consisting of 4-40 amino acids connected via peptide bonds. In one embodiment, the spacer is a peptide segment consisting of 4-24 amino acids connected via peptide bonds.

In one embodiment, the spacer comprises one or more of the following amino acid residues: Gly (G), Glu (E), Ser (S), Pro (P), Arg (R), Phe (F), Tyr (Y), Asp (D), and Lys (K). In one embodiment, the spacer comprises one or more of the following amino acid residues: Gly (G), Glu (E), Ser (S), and Lys (K). In one embodiment, the spacer comprises one or more of the following amino acid residues: Gly (G), Ser (S), Pro (P), Arg (R), Phe (F), Tyr (Y), Asp (D), and Lys (K). In one embodiment, the spacer comprises one or more of the following amino acid residues: Gly (G), Ser (S), Pro (P), and Lys (K). In one embodiment, the spacer comprises at least one Lys (K) residue.

In one embodiment, the human insulin or human insulin analogue of the invention comprises a peptide spacer at the C-terminal of the A-chain of said human insulin or said human insulin analogue. In one embodiment, said peptide spacer comprises (GES)ₚK, wherein p is an integer from 3 to 12.

Examples of peptide spacers at the C-terminal of the A-chain of said human insulin or said human insulin analogue include: (GES)₃K (SEQ ID NO:29); (GES)₆K (SEQ ID NO:30); and (GES)₁₂K (SEQ ID NO:31).

In one embodiment, the human insulin or human insulin analogue of the invention comprises a peptide spacer at the N-terminal of the B-chain of said human insulin or said human insulin analogue. In one embodiment, said peptide spacer comprises GKPG, GKP(G₄S)_{q}, KP(G₄S)ᵣ, GKPRGFFYTP(G₄S)ₛ, or TYFFGRKPD(G₄S)ₜ, wherein each of q, r, s and t is independently selected from an integer from 1 to 5. In another embodiment, the peptide spacer comprises GKPG, GKP(G₄S)_{q}, KP(G₄S)₃, GKPRGFFYTP(G₄S)₂, or TYFFGRKPD(G₄S)₃, wherein q is an integer from 1 to 3.

Examples of peptide spacers at the N-terminal of the B-chain of said human insulin or said human insulin analogue include:

(SEQ ID NO: 32)
GKPG;
(SEQ ID NO: 33)
GKPGGGGS (GKP(G₄S));
(SEQ ID NO: 34)
GKPGGGGSGGGGS (GKP(G₄S)₂);
(SEQ ID NO: 35)
GKPGGGGSGGGGSGGGGS (GKP(G₄S)₃);
(SEQ ID NO: 36)
KPGGGGSGGGGSGGGGS (KP(G₄S)₃);
(SEQ ID NO: 37)
GKPRGFFYTPGGGGSGGGGS (GKPRGFFYTP(G₄S)₂);
and
(SEQ ID NO: 38)
TYFFGRKPDGGGGSGGGGSGGGGS (TYFFGRKPD(G₄S)₃).

Examples of insulin analogues comprising a peptide spacer at the C-terminal of the A-chain of said human insulin or said human insulin analogue include:

A21Q (GES)₃K desB30 human insulin (SEQ ID NO:8 and SEQ ID NO:11);

A21Q (GES)₆K desB30 human insulin (SEQ ID NO:9 and SEQ ID NO:11); and

A21Q (GES)₁₂K desB30 human insulin (SEQ ID NO:10 and SEQ ID NO:11).

Examples of insulin analogues comprising a peptide spacer at the N-terminal of the B-chain of said human insulin or said human insulin analogue include:

(SEQ ID NO: 1 and SEQ ID NO: 21)
B1-KPGGGGSGGGGSGGGGS desB30 human insulin;
(SEQ ID NO: 4 and SEQ ID NO: 22)
B1-KPGGGGSGGGGSGGGGS A14E B25H desB30 human
insulin;
(SEQ ID NO: 1 and SEQ ID NO: 23)
B1-GKPGGGGSGGGGSGGGGS desB30 human insulin;
(SEQ ID NO: 1 and SEQ ID NO: 24)
B1-GKPGGGGSGGGGS desB30 human insulin;
(SEQ ID NO: 1 and SEQ ID NO: 25)
B1-GKPGGGGS desB30 human insulin;
(SEQ ID NO: 1 and SEQ ID NO: 26)
B1-GKPG desB30 human insulin;
(SEQ ID NO: 1 and SEQ ID NO: 27)
B1-GKPRGFFYTPGGGGSGGGGS desB30 human insulin;
and
(SEQ ID NO: 1 and SEQ ID NO: 28)
B1-TYFFGRKPDGGGGSGGGGSGGGGS desB30 human insulin.

Linker L

In one aspect, the spacer is a non-peptide linker L. Various non-peptide linkers are known in the art, and may be used in the compounds of the present invention.

In one embodiment, the human insulin or human insulin analogue of the invention comprises a linker L at the N-terminal of the B-chain of said human insulin or said human insulin analogue.

In one embodiment, the linker is of Formula L1:

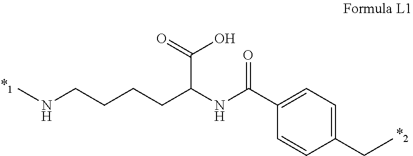

wherein *1 denotes the attachment point to the modifying group M and *2 denotes the attachment point to the amino group of the amino acid residue at the N-terminal of the B-chain of human insulin or the human insulin analogue.

In one embodiment, the linker is of formula L2:

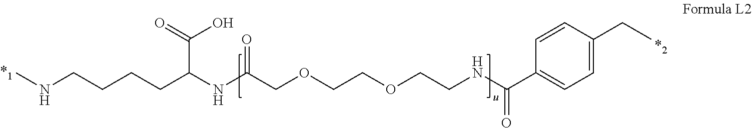

wherein *1 denotes the attachment point to the modifying group A and *2 denotes the attachment point to the amino group of the amino acid residue at N-terminal of the B-chain of human insulin or the human insulin analogue, and wherein u is 1, 2 or 3. In one embodiment, u is 2 or 3.

In one embodiment, the linker is of formula L3:

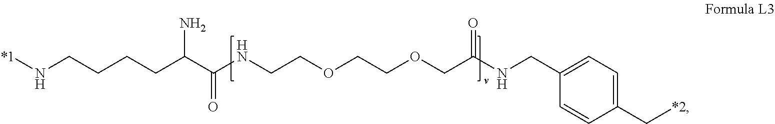

wherein *1 denotes the attachment point to the modifying group A and *2 denotes the attachment point to the amino group of the amino acid residue at N-terminal of the B-chain of human insulin or the human insulin analogue, and wherein v is 2 or 3.

Insulin Derivative

The term “insulin derivative” as used herein means a chemically modified insulin or an analogue thereof, wherein the modification(s) are in the form of attachment of one or more modifying groups M.

In one embodiment, each of the one or more modifying groups M is attached, optionally via a spacer, to the amino group of the N-terminal amino acid residue of the A-chain or B-chain of said human insulin or human insulin analogue or to the epsilon amino group of a lysine in said human insulin or human insulin analogue.

In one embodiment, each modifying group M is attached to an attachment point selected from one of the following groups:
a) the amino group of the N-terminal amino acid residue of the A-chain of said human insulin or human insulin analogue;
b) the epsilon amino group of a lysine in position 22 of the A-chain of said human insulin analogue; or the epsilon amino group of the lysine in said optional peptide spacer at the C-terminal of the A-chain of said human insulin or human insulin analogue;
c) the amino group of the N-terminal amino acid residue of the B-chain of said human insulin or human insulin analogue;
   the epsilon amino group of a lysine residue in position 1 or position 4 of the B-chain of said human insulin analogue;
   the epsilon amino group of a lysine in said optional peptide spacer at the N-terminal of the B-chain of said human insulin or human insulin analogue; or the distal amino group marked with *1 in said optional linker L at the N-terminal of the B-chain of said human insulin or human insulin analogue; and
d) the epsilon amino group of a lysine in position 22 or position 29 of the B-chain of said human insulin or human insulin analogue.

In one embodiment, not more than one modifying group M is attached to a point of attachment within each of the groups a), b), c) and d).

In one embodiment, the compound of the invention comprises two modifying groups M, wherein one modifying group M is attached to the amino group of a lysine residue in position 1 or position 4 of the B-chain of the human insulin analogue, or the epsilon amino group of a lysine in the optional peptide extension at the N-terminal of the B-chain of the human insulin or the human insulin analogue; and the other modifying group M is attached to the epsilon amino group of a lysine in position 29 of the B-chain of the human insulin or the human insulin analogue.

In one embodiment, the compound of the invention has exactly two modifying groups M, wherein one modifying group M is attached to the amino group of a lysine residue in position 1 or position 4 of the B-chain of the human insulin analogue, or the epsilon amino group of a lysine in the optional peptide extension at the N-terminal of the B-chain of the human insulin or the human insulin analogue; and the other modifying group M is attached to the epsilon amino group of a lysine in position 29 of the B-chain of the human insulin or the human insulin analogue.

In one embodiment, the compound of the invention comprises two modifying groups M, wherein one modifying group M is attached to the amino group of the N-terminal amino acid residue of the A-chain of said human insulin or human insulin analogue; and the other modifying group M is attached to the epsilon amino group of a lysine in position 29 of the B-chain of said human insulin or human insulin analogue.

In one embodiment, the compound of the invention has exactly two modifying groups M, wherein one modifying group M is attached to the amino group of the N-terminal amino acid residue of the A-chain of said human insulin or human insulin analogue; and the other modifying group M is attached to the epsilon amino group of a lysine in position 29 of the B-chain of said human insulin or human insulin analogue.

In one embodiment, the compound of the invention comprises two modifying groups M, wherein one modifying group M is attached to the epsilon amino group of a lysine in position 22 of the A-chain of said human insulin analogue, or to the epsilon amino group of the lysine in the optional peptide spacer at the C-terminal of the A-chain of said human insulin or human insulin analogue; and the other modifying group M is attached to the epsilon amino group of a lysine in position 22 or position 29 of the B-chain of said human insulin or human insulin analogue.

In one embodiment, the compound of the invention has exactly two modifying groups M, wherein one modifying group M is attached to the epsilon amino group of a lysine in position 22 of the A-chain of said human insulin analogue, or to the epsilon amino group of the lysine in the optional peptide spacer at the C-terminal of the A-chain of said human insulin or human insulin analogue; and the other modifying group M is attached to the epsilon amino group of a lysine in position 22 or position 29 of the B-chain of said human insulin or human insulin analogue.

In one embodiment, the compound of the invention comprises one modifying group M, wherein the modifying group M is attached to the epsilon amino group of a lysine in position 22 of the A-chain of said human insulin analogue; or to the epsilon amino group of a lysine in position 29 of the B-chain of said human insulin or human insulin analogue.

In one embodiment, the compound of the invention has exactly one modifying group M, wherein the modifying group M is attached to the epsilon amino group of a lysine in position 22 of the A-chain of said human insulin analogue; or to the epsilon amino group of a lysine in position 29 of the B-chain of said human insulin or human insulin analogue.

In one embodiment, the compound of the invention comprises three or four modifying groups M, wherein a first modifying group M is attached to the epsilon amino group of a lysine in position 22 of the A-chain of said human insulin analogue; a second modifying group M is attached to the epsilon amino group of a lysine in position 22 or position 29 of the B-chain of said human insulin or human insulin analogue; with the remaining modifying groups M each being attached to either the amino group of the N-terminal amino acid residue of the A-chain of said human insulin or human insulin analogue; to the epsilon amino group of a lysine in position 22 or position 29 of the B-chain of said human insulin or human insulin analogue; or to the distal amino group marked with *1 in said optional linker L at the N-terminal of the B-chain of said human insulin or human insulin analogue.

In one embodiment, the compound of the invention has exactly three or four modifying groups M, wherein a first modifying group M is attached to the epsilon amino group of a lysine in position 22 of the A-chain of said human insulin analogue; a second modifying group M is attached to the epsilon amino group of a lysine in position 22 or position 29 of the B-chain of said human insulin or human insulin analogue; with the remaining modifying groups M each being attached to either the amino group of the N-terminal amino acid residue of the A-chain of said human insulin or human insulin analogue; to the epsilon amino group of a lysine in position 22 or position 29 of the B-chain of said human insulin or human insulin analogue; or to the distal amino group marked with *1 in said optional linker L at the N-terminal of the B-chain of said human insulin or human insulin analogue.

Modifying Group M

The compounds of the present invention comprise one or more modifying groups M. In one embodiment, the compound of the present invention comprises one, two, three or four modifying groups M. In one embodiment, the compound of the present invention comprise two or more modifying groups M. In one embodiment, the compound of the present invention comprises two, three or four modifying groups M. In one embodiment, the compound of the present invention comprises two modifying groups M. In one embodiment, the compound of the present invention has exactly two modifying groups M. The one or more modifying groups may be identical or different. The two or more modifying groups may be identical or different. In one embodiment, the modifying groups are identical.

Some of the modifying groups comprises one or more amino acid residues. Each of these amino acid residues can independently be the D- or the L-form of the respective amino acid residue, i.e. each of the chiral atoms in the modifying groups can independently be of the (R)- or (S)-form. In one embodiment, the amino acid residues of the modifying groups are L-amino acid residues.

Each modifying group M comprise a diboron moiety, wherein the diboron moiety (i.e. the modifying group M) comprises two aryl moieties, wherein a boron atom is attached to each of the two aryl moieties. The boron atom can be part of a boronic acid (or boronate depending on pKa/pH), or it can be part of a boroxole (or boroxolate depending on pKa/pH).

The terms “comprises” or “comprising” certain features are to be interpreted as meaning that the subject matter in question includes those certain features, but that it does not exclude the presence of other features. Thus, a modifying group M may have more than two aryl moieties, wherein a boron atom is attached to each of the aryl moieties. In one embodiment, the modifying group has exactly two aryl moieties, wherein a boron atom is attached to each of the two aryl moieties. In one embodiment, the modifying group has exactly four aryl moieties, wherein a boron atom is attached to each of the four aryl moieties.

The diboronates/diboroxoles of the present invention binds glucose stronger than monoboronates, as shown in Example A. Moreover, surprisingly, the diboron compounds of the invention are capable of binding to human serum albumin (HSA), thus possessing a dual action, as the HSA binding binding also is glucose-sensitive (the HSA-bound fraction of the diboron peptide is inactive due to blocking of the receptor binding sites on the peptide) (data shown in Example B).

In one embodiment, the modifying group is of formula M1:

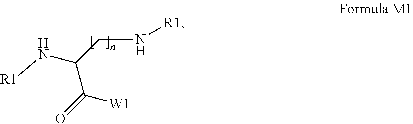

which represents a D- or an L-amino acid form, and

wherein n represents an integer in the range of 1 to 4;

wherein W1 is absent and represents the point of attachment * to said human insulin or human insulin analogue, or W1 represents

NH—CH₂—C(═O)—*,

NH—CH₂CH₂—C(═O)—*,

the D- or L-form of NH—CH(COOH)—CH₂CH₂—C(═O)—*,

the D- or L-form of NH—CH(COOH)—CH₂CH₂—C(═O)—NH—CH₂CH₂—C(═O)—*, or

NH—CH₂CH₂—C(═O)—NH—(CH₂)₂—O—(CH₂)₂—O—CH₂—CO—*,

wherein * represents the point of attachment to said human insulin or human insulin analogue; and

wherein R1 is selected from

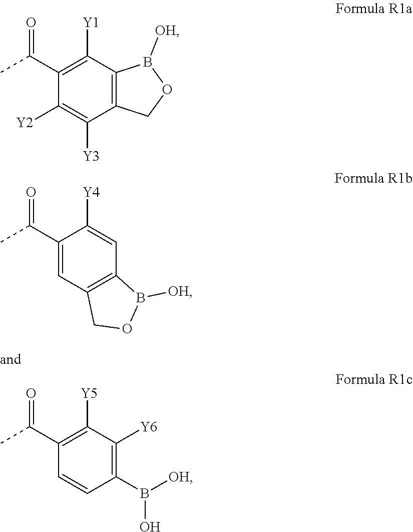

wherein Y1, Y2, Y3, Y4, Y5 and Y6 is independently selected from H, F, Cl, CHF₂, and CF₃.

In another embodiment, the modifying group is of formula M1, wherein Y1 and Y2 is H, and Y3 is F or CF₃; Y4 is H or F; and Y5 is H and Y6 is F.

In yet another embodiment, the modifying group is of formula M1, wherein n is 1; W1 represents NH—CH₂CH₂—C(═O)—* or the L-form of NH—CH(COOH)—CH₂CH₂—C(═O)—*, wherein * represents the point of attachment to said human insulin or human insulin analogue; and R1 is of

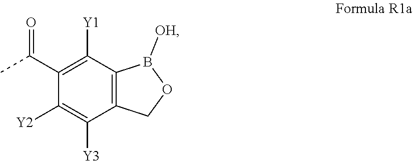

wherein Y1 and Y2 are H; and Y3 is F or CF₃.

In one embodiment, the modifying group is of formula M2:

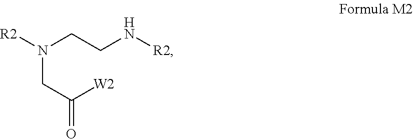

wherein W2 is absent and represents the point of attachment * to said human insulin or human insulin analogue, or W2 represents the D- or L-form of NH—CH(COOH)—CH₂CH₂—C(═O)—*, or NH—CH₂CH₂CH₂—C(═O)—*, wherein * represents the point of attachment to said human insulin or human insulin analogue; and

wherein R2 is selected from

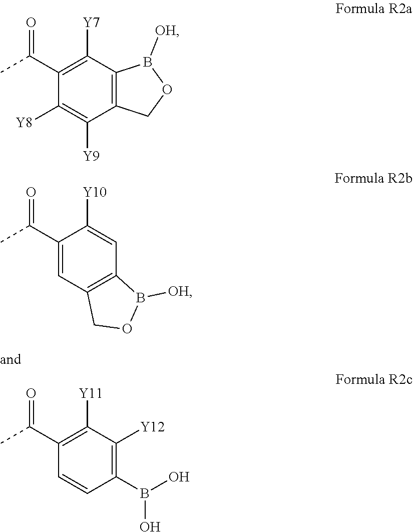

wherein Y7, Y8, Y9, Y10, Y11 and Y12 are independently selected from H, F, Cl, CHF₂, and CF₃.

In another embodiment, the modifying group is of formula M2, wherein Y7 is H; Y8 is H, Cl, CHF₂, or CF₃; Y9 is H, F, or CF₃; Y10 is F; Y11 is H; and Y12 is F; with the provisio that only one of Y8 and Y9 is H.

In yet another embodiment, the modifying group is of formula M2, wherein W2 is absent and represents the point of attachment * to said human insulin or human insulin analogue, or W2 represents the L-form of NH—CH(COOH)—CH₂CH₂—C(═O)—*, wherein * represents the point of attachment to said human insulin or human insulin analogue; and wherein R2 is of

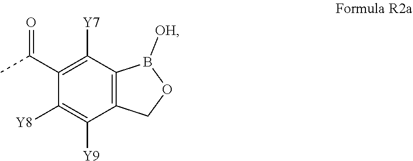

wherein Y7 and Y8 are H; and Y9 is CI, CHF₂, or CF3.

In one embodiment, the modifying group is of formula M3:

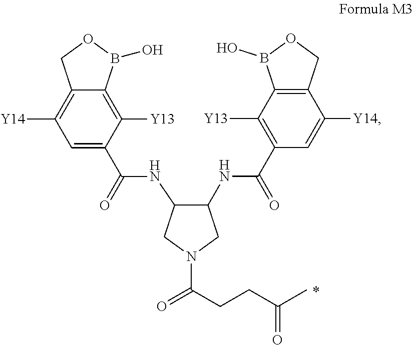

which represents a R,R or S,S or R,S stereoisomer of the 3,4-diamino-pyrrolidine; and

wherein * represents the point of attachment to said human insulin or human insulin analogue; and wherein Y13 and Y14 are independently selected from H, F, Cl, CHF₂, and CF₃.

In another embodiment, the modifying group is of formula M3, wherein Y13 is H or F; and Y14 is H or CF₃; with the provisio that only one of Y13 and Y14 is H.

In one embodiment, the modifying group is of formula M4:

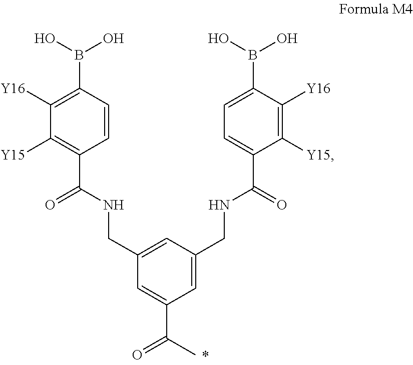

wherein * represents the point of attachment to said human insulin or human insulin analogue, and wherein Y15 and Y16 is independently selected from H, F, Cl, CHF₂, and CF₃.

In another embodiment, the modifying group is of formula M4, wherein Y15 and Y16 is independently selected from H, and F.

In yet another embodiment, the modifying group is of formula M4, wherein Y15 is H, and Y16 is F.

In one embodiment, the modifying group is of formula M5:

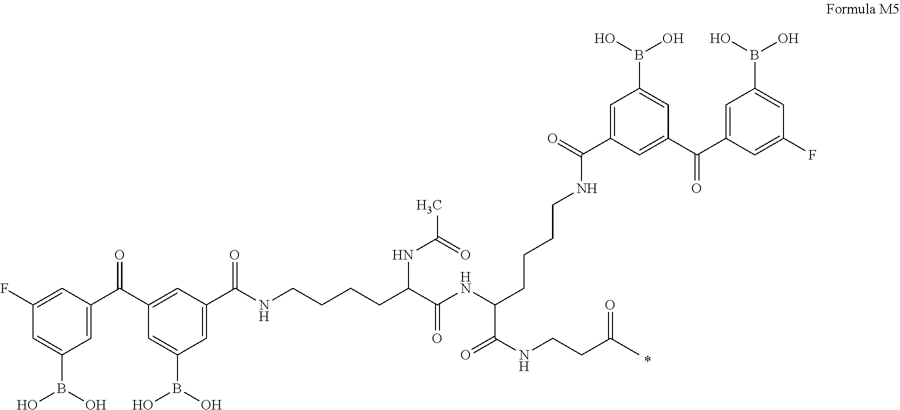

wherein each of the amino acid residues independently represents a D- or an L-amino acid form, and wherein * represents the point of attachment to said human insulin or human insulin analogue.

In one embodiment, the modifying group is of formula M6:

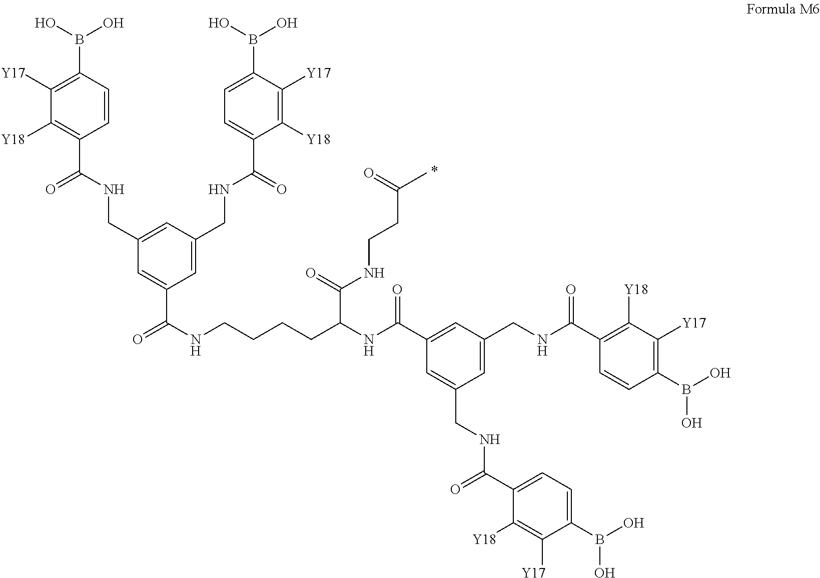

wherein the α-amino acid residue represents a D- or an L-amino acid form, and wherein * represents the point of attachment to said human insulin or human insulin analogue; and

wherein Y17 and Y18 is independently selected from H, F, Cl, CHF₂, and CF₃.

In another embodiment, the modifying group is of formula M6, wherein Y17 is H or F; and Y18 is H or F.

In one embodiment, the modifying group is of formula M7:

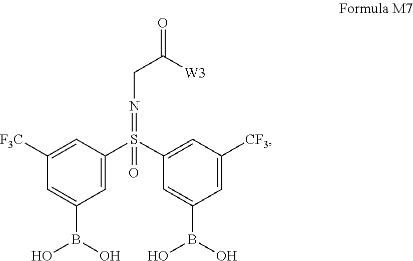

wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue. In one embodiment, W3 represents the L-form of NH—CH(COOH)—CH₂CH₂—C(═O)—*, wherein * represents the point of attachment to said human insulin or human insulin analogue.

In one embodiment, the modifying group is of formula M8:

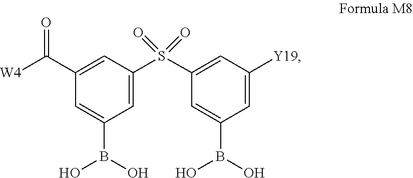

wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is H, F, Cl, CHF₂, and CF₃ or SF₅.

In another embodiment, the modifying group is of formula M8, wherein Y19 is CF₃ or SF₅.

In yet another embodiment, the modifying group is of formula M8, wherein Y19 is CF₃.

In one embodiment, the modifying group is of formula M9:

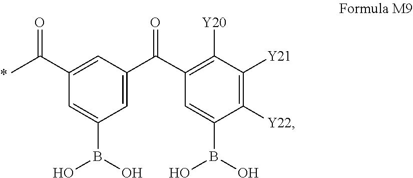

wherein * represents the point of attachment to said human insulin or human insulin analogue; and wherein each of Y20, Y21, and Y22 is independently selected from H, F, Cl, CHF₂, and CF₃.

In another embodiment, the modifying group is of formula M9, wherein each of Y20, Y21, and Y22 is independently selected from H, and F; with the provisio that when Y21 is F, then Y20 and Y22 are H; and when Y21 is H, then Y20 and Y22 are F.

In one embodiment, the modifying group is of formula M10:

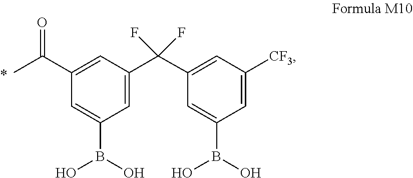

wherein * represents the point of attachment to said human insulin or human insulin analogue.

In one embodiment, the modifying group is of formula M11:

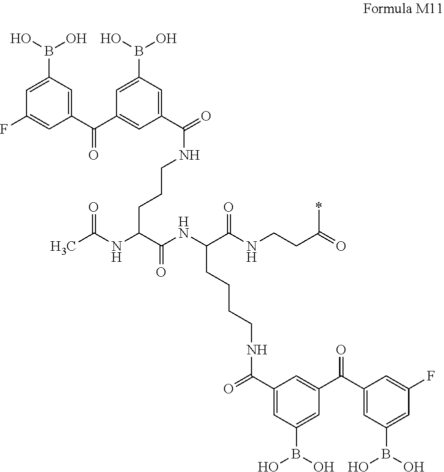 [SMILES: *C(=O)CCNC(=O)C(CCCCNC(=O)c1cc(B(O)O)cc(C(=O)c2cc(F)cc(B(O)O)c2)c1)NC(=O)C(CCCNC(=O)c1cc(B(O)O)cc(C(=O)c2cc(F)cc(B(O)O)c2)c1)NC(C)=O]

wherein each of the amino acid residues represents a D- or an L-amino acid form, and

wherein * represents the point of attachment to said human insulin or human insulin analogue.

Compounds of the Present, Invention

In one embodiment, the compound of the invention comprises human insulin or a human insulin analogue; and one or more modifying groups M, wherein each of the modifying groups M comprises two aryl moieties, wherein a boron atom is attached to each of the two aryl moieties; and wherein each of the one or more modifying groups M is attached, optionally via a spacer, to the amino group of the N-terminal amino acid residue of the A-chain or B-chain of said human insulin or human insulin analogue or to the epsilon amino group of a lysine in said human insulin or human insulin analogue.

In another embodiment, the compound of the invention comprises human insulin or a human insulin analogue; and 2 modifying groups M, wherein each of the modifying groups M comprises two aryl moieties, wherein a boron atom is attached to each of the two aryl moieties; and wherein a first modifying group M is attached to the epsilon amino group of a lysine residue in position 1 or position 4 of the B-chain of said human insulin analogue, or to the epsilon amino group of a lysine in an optional peptide spacer at the N-terminal of the B-chain of said human insulin or human insulin analogue; and a second modifying group is attached to the epsilon amino group of a lysine in position 22 or position 29 of the B-chain of said human insulin or human insulin analogue.

In another embodiment, the compound of the invention comprises human insulin or a human insulin analogue; and 2 modifying groups M, wherein each of the modifying groups M comprises two aryl moieties, wherein a boron atom is attached to each of the two aryl moieties; and wherein a first modifying group M is attached to the amino group of the N-terminal amino acid residue of the A-chain of said human insulin or human insulin analogue; and a second modifying group is attached to the epsilon amino group of a lysine in position 29 of the B-chain of said human insulin or human insulin analogue.

In another embodiment, the compound of the invention comprises human insulin or a human insulin analogue; and 2 modifying groups M, wherein each of the modifying groups M comprises two aryl moieties, wherein a boron atom is attached to each of the two aryl moieties; and wherein a first modifying group M is attached to the epsilon amino group of a lysine in position 22 of the A-chain of said human insulin analogue, or to the epsilon amino group of the lysine in an optional peptide spacer at the C-terminal of the A-chain of said human insulin or human insulin analogue; and a second modifying group is attached to the epsilon amino group of a lysine in position 22 or position 29 of the B-chain of said human insulin or human insulin analogue.

In another embodiment, the compound of the invention comprises human insulin or a human insulin analogue; and 1 modifying group M, wherein the modifying group M comprises two aryl moieties, wherein a boron atom is attached to each of the two aryl moieties; and wherein the modifying group M is attached to the epsilon amino group of a lysine in position 22 of the A-chain of said human insulin analogue, or to the epsilon amino group of a lysine in position 22 or position 29 of the B-chain of said human insulin or human insulin analogue.

In one embodiment, the invention relates to compounds independently selected from the group of compounds of examples 181, 205, 210, 211, 227, 233, 234, 239, 240, 241, 272, 273, 280, 284, 285, 288, 291, 300, 301, 324, 327, 331, 333, and 335.

In one embodiment, the invention relates to compounds independently selected from the group of compounds of examples 181, 205, 210, 211, 227, 233, 234, 239, 240, 241, 272, 273, 280, 285, 288, 291, 300, 301, 327, 331, 333, and 335.

In one embodiment, the compound of the invention is the compound of Example 181. In one embodiment, the compound of the invention is the compound of 205. In one embodiment, the compound of the invention is the compound of 210. In one embodiment, the compound of the invention is the compound of 211. In one embodiment, the compound of the invention is the compound of 227. In one embodiment, the compound of the invention is the compound of 233. In one embodiment, the compound of the invention is the compound of 234. In one embodiment, the compound of the invention is the compound of 239. In one embodiment, the compound of the invention is the compound of 240. In one embodiment, the compound of the invention is the compound of 241. In one embodiment, the compound of the invention is the compound of 272. In one embodiment, the compound of the invention is the compound of 273. In one embodiment, the compound of the invention is the compound of 280. In one embodiment, the compound of the invention is the compound of 284. In one embodiment, the compound of the invention is the compound of 285. In one embodiment, the compound of the invention is the compound of 288. In one embodiment, the compound of the invention is the compound of 291. In one embodiment, the compound of the invention is the compound of 300. In one embodiment, the compound of the invention is the compound of 301. In one embodiment, the compound of the invention is the compound of 324. In one embodiment, the compound of the invention is the compound of 327. In one embodiment, the compound of the invention is the compound of 331. In one embodiment, the compound of the invention is the compound of 333. In one embodiment, the compound of the invention is the compound of 335.

Intermediate Products

The invention furthermore provides an intermediate product in the form of a novel insulin analogue or an insulin analogue comprising a peptide spacer.

The invention thus also relates to intermediate products independently selected from the group consisting of:

A14E desB1-B2 B4K B5P desB30 human insulin (SEQ ID NO:4 and SEQ ID NO:16);

A14E desB1-B2 B3G B4K B5P desB30 human insulin (SEQ ID NO:4 and SEQ ID NO:17);

A14E B-1G B1K B2P desB30 human insulin (SEQ ID NO:4 and SEQ ID NO:18);

A22K B22K B29R desB30 human insulin (SEQ ID NO:6 and SEQ ID NO:20);

A21Q (GES)3K desB30 human insulin (SEQ ID NO:8 and SEQ ID NO:11);

A21Q (GES)6K desB30 human insulin (SEQ ID NO:9 and SEQ ID NO:11);

A21Q (GES)12K desB30 human insulin (SEQ ID NO:10 and SEQ ID NO:11);

B1-KPGGGGSGGGGSGGGGS desB30 human insulin (SEQ ID NO:1 and SEQ ID NO:21);

B1-KPGGGGSGGGGSGGGGS A14E B25H desB30 human insulin (SEQ ID NO:4 and SEQ ID NO:22);

B1-GKPGGGGSGGGGSGGGGS desB30 human insulin (SEQ ID NO:1 and SEQ ID NO:23);

B1-GKPGGGGSGGGGS desB30 human insulin (SEQ ID NO:1 and SEQ ID NO:24);

B1-GKPGGGGS desB30 human insulin (SEQ ID NO:1 and SEQ ID NO:25);

B1-GKPG desB30 human insulin (SEQ ID NO:1 and SEQ ID NO:26);

B1-GKPRGFFYTPGGGGSGGGGS desB30 human insulin (SEQ ID NO:1 and SEQ ID NO:27); and

B1-TYFFGRKPDGGGGSGGGGSGGGGS desB30 human insulin (SEQ ID NO:1 and SEQ ID NO:28).

Insulin Function

The relative binding affinity of insulin analogues for the human insulin receptor (IR) can be determined by competition binding in a scintillation proximity assay (SPA) as described in Example B.

In one embodiment the compounds of the invention have the ability to bind to the insulin receptor. In one embodiment, the compounds of the invention have higher insulin receptor affinity in presence of 20 mM glucose than when no glucose is present.

The AKT phosphorylation assay described in Example C and the lipogenesis assay described in Example D can be used as a measure of the functional (agonistic) activity of an insulin analogue.

Pharmaceutical Composition

The invention also relates to pharmaceutical compositions comprising a compound of the invention, including e.g. an analogue of the invention, or a pharmaceutically acceptable salt, amide, or ester thereof, and one or more pharmaceutically acceptable excipient (s). Such compositions may be prepared as is known in the art.

The term “excipient” broadly refers to any component other than the active therapeutic ingredient(s). The excipient may be an inert substance, an inactive substance, and/or a not medicinally active substance. The excipient may serve various purposes, e.g. as a carrier, vehicle, diluent, and/or to improve administration, and/or absorption of the active substance. Non-limiting examples of excipients are: solvents, diluents, buffers, preservatives, tonicity regulating agents, chelating agents, and stabilisers. The formulation of pharmaceutically active ingredients with various excipients is known in the art, see e.g. Remington: The Science and Practice of Pharmacy (e.g. 21st edition (2005), and any later editions).

A composition of the invention may be in the form of a liquid formulation, i.e. aqueous formulation comprising water. A liquid formulation may be a solution, or a suspension. A composition of the invention may be for parenteral administration, e.g. performed by subcutaneous, intramuscular, intraperitoneal, or intravenous injection.

Aryl boron compounds generally have low stability in aqueous solutions at pH near neutral value. The C—B bond can hydrolyse to give the phenyl residue and free borate, Ph-H+B(OH)₃, or the compound can be oxidized to give the phenolic residue+free borate, Ph-OH+B(OH)₃. Certain preferred diboron compounds and diboron insulin conjugates of the invention are found to be more stable than other aryl-borons of the invention and aryl-borons in general. Stability can for instance be assessed by measuring the purity of the insulin derivatives after standing in aqueous solution at neutral pH at 25° or 37° Celcius for an extended period of time, for instance a week.

Pharmaceutical Indications

Diabetes

The term “diabetes” or “diabetes mellitus” includes type 1 diabetes, type 2 diabetes, gestational diabetes (during pregnancy) and other states that cause hyperglycaemia. The term is used for a metabolic disorder in which the pancreas produces insufficient amounts of insulin, or in which the cells of the body fail to respond appropriately to insulin thus preventing cells from absorbing glucose. As a result, glucose builds up in the blood.

Type 1 diabetes, also called insulin-dependent diabetes mellitus (IDDM) and juvenile-onset diabetes, is caused by beta-cell destruction, usually leading to absolute insulin deficiency. Type 2 diabetes, also known as non-insulin-dependent diabetes mellitus (NIDDM) and adult-onset diabetes, is associated with predominant insulin resistance and thus relative insulin deficiency and/or a predominantly insulin secretory defect with insulin resistance.

Other Indications

In one embodiment, a compound according to the invention is used for the preparation of a medicament for the treatment or prevention of hyperglycemia including stress induced hyperglycemia, type 2 diabetes, impaired glucose tolerance, or type 1 diabetes.

In another embodiment, a compound according to the invention is used as a medicament for delaying or preventing disease progression in type 2 diabetes.

In one embodiment of the invention, the compound is for use as a medicament for the treatment or prevention of hyperglycemia including stress induced hyperglycemia, type 2 diabetes, impaired glucose tolerance, or type 1 diabetes.

In a further embodiment the invention is related to a method for the treatment or prevention of hyperglycemia including stress induced hyperglycemia, type 2 diabetes, impaired glucose tolerance, or type 1 diabetes, the method comprising administering to a patient in need of such treatment an effective amount for such treatment of a compound according to the invention.

Mode of Administration

The term “treatment” is meant to include both the prevention and minimization of the referenced disease, disorder, or condition (i.e., “treatment” refers to both prophylactic and therapeutic administration of a compound of the present invention or a composition comprising a compound of the present invention unless otherwise indicated or clearly contradicted by context).

The route of administration may be any route which effectively transports a compound of this invention to the desired or appropriate place in the body, such as parenterally, for example, subcutaneously, intramuscularly or intraveneously.

For parenterally administration, a compound of this invention is formulated analogously with the formulation of known insulins. Furthermore, for parenterally administration, a compound of this invention is administered analogously with the administration of known insulins and the physicians are familiar with this procedure.

The amount of a compound of this invention to be administered, the determination of how frequently to administer a compound of this invention, and the election of which compound or compounds of this invention to administer, optionally together with another antidiabetic compound, is decided in consultation with a practitioner who is familiar with the treatment of diabetes.

While certain features of the invention have been illustrated and described herein, many modifications, substitutions, changes, and equivalents will now occur to those of ordinary skill in the art. It is, therefore, to be understood that the appended embodiments are intended to cover all such modifications and changes as fall within the true spirit of the invention.

### EMBODIMENTS

The invention is further described by the following non-limiting embodiments of the invention:

1. A compound comprising

i) human insulin or a human insulin analogue; and

ii) one or more modifying groups, wherein each of the modifying groups M comprises two aryl moieties, wherein a boron atom is attached to each of the two aryl moieties; and

wherein each of the one or more modifying groups M is attached, optionally via a spacer, to the amino group of the N-terminal amino acid residue of the A-chain or B-chain of said human insulin or human insulin analogue or to the epsilon amino group of a lysine in said human insulin or human insulin analogue.

2. The compound according to embodiment 1, wherein each of the modifying groups is independently selected from the group of

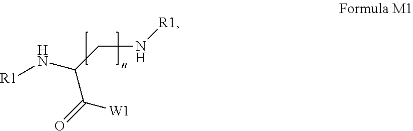

which represents a D- or an L-amino acid form, and

wherein n represents an integer in the range of 1 to 4;

wherein W1 is absent and represents the point of attachment * to said human insulin or human insulin analogue, or W1 represents
NH—CH₂—C(═O)—*,
NH—CH₂CH₂—C(═O)—*,
the D- or L-form of NH—CH(COOH)—CH₂CH₂—C(═O)—*,
the D- or L-form of NH—CH(COOH)—CH₂CH₂—C(═O)—NH—CH₂CH₂—C(═O)—*, or
NH—CH₂CH₂—C(═O)—NH—(CH₂)₂—O—(CH₂)₂—O—CH₂—CO—*,

wherein * represents the point of attachment to said human insulin or human insulin analogue; and

wherein R1 is selected from

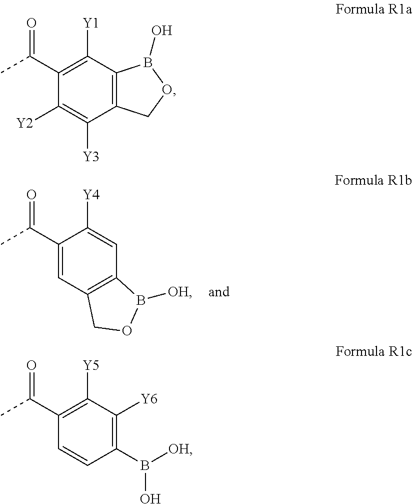

wherein Y1, Y2, Y3, Y4, Y5 and Y6 is independently selected from H, F, Cl, CHF₂, and CF₃;

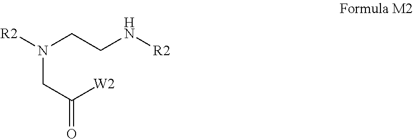

wherein W2 is absent and represents the point of attachment * to said human insulin or human insulin analogue, or W2 represents the D- or L-form of NH—CH(COOH)—CH₂CH₂—C(═O)—*, or NH—CH₂CH₂CH₂—C(═O)—*, wherein * represents the point of attachment to said human insulin or human insulin analogue; and wherein R2 is selected from

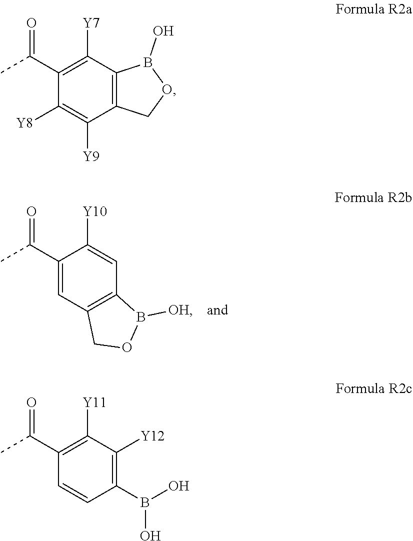

wherein Y7, Y8, Y9, Y10, Y11 and Y12 is independently selected from H, F, Cl, CHF₂, and CF₃;

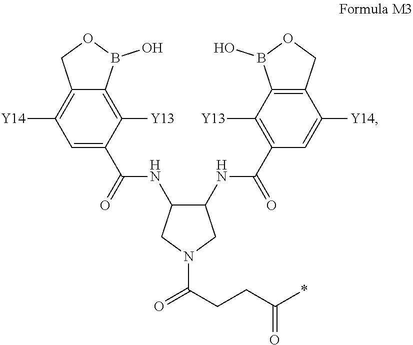

which represents a R,R or S,S or R,S or stereoisomer of the 3,4-diamino-pyrrolidine; and

wherein * represents the point of attachment to said human insulin or human insulin analogue; and wherein Y13 and Y14 is independently selected from H, F, Cl, CHF₂, and CF₃;

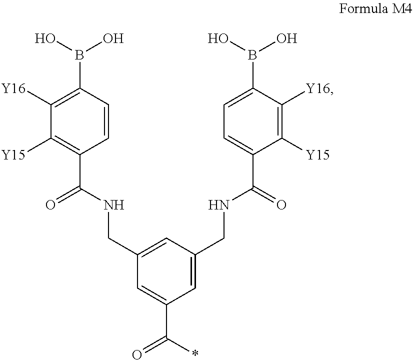

wherein * represents the point of attachment to said human insulin or human insulin analogue, and wherein Y15 and Y16 is independently selected from H, F, Cl, CHF₂, and CF₃;

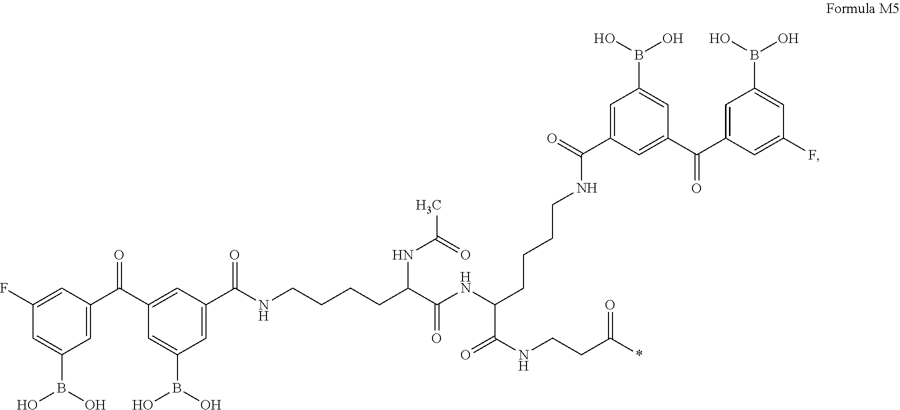 [SMILES: *C(=O)CCNC(=O)C(CCCCNC(=O)c1cc(B(O)O)cc(C(=O)c2cc(F)cc(B(O)O)c2)c1)NC(=O)C(CCCCNC(=O)c1cc(B(O)O)cc(C(=O)c2cc(F)cc(B(O)O)c2)c1)NC(C)=O]

wherein each of the amino acid residues independently represents a D- or an L-amino acid form, and wherein * represents the point of attachment to said human insulin or human insulin analogue;

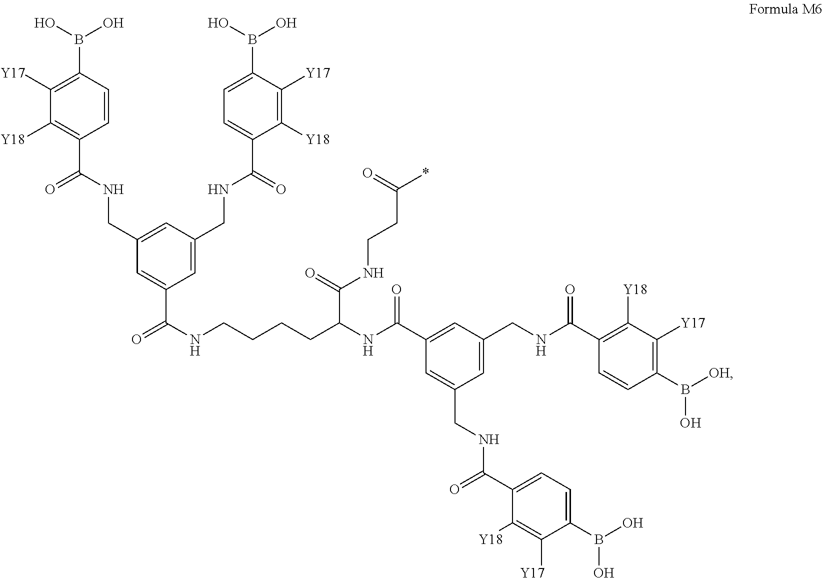

wherein the α-amino acid residue represents a D- or an L-amino acid form, and wherein * represents the point of attachment to said human insulin or human insulin analogue; and

wherein Y17 and Y18 is independently selected from H, F, Cl, CHF₂, and CF₃;

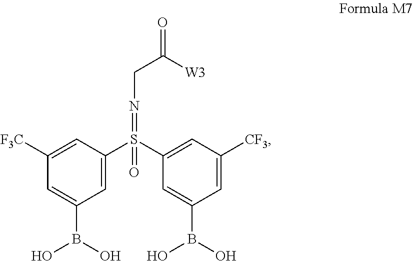

wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue;

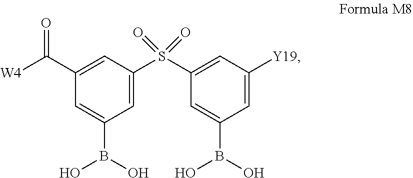

wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is H, F, Cl, CHF₂, and CF₃ or SF₅;

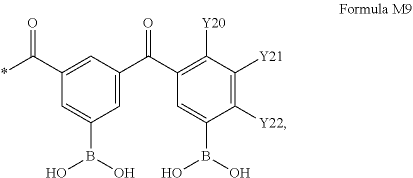

wherein * represents the point of attachment to said human insulin or human insulin analogue; and wherein each of Y20, Y21, and Y22 is independently selected from H, F, Cl, CHF₂, and CF₃;

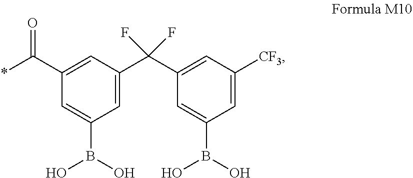 [SMILES: *C(=O)c1cc(B(O)O)cc(C(F)(F)c2cc(B(O)O)cc(C(F)(F)F)c2)c1]

wherein * represents the point of attachment to said human insulin or human insulin analogue; and

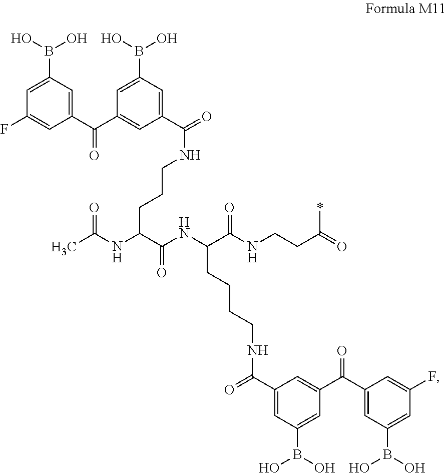 [SMILES: *C(=O)CCNC(=O)C(CCCCNC(=O)c1cc(B(O)O)cc(C(=O)c2cc(F)cc(B(O)O)c2)c1)NC(=O)C(CCCNC(=O)c1cc(B(O)O)cc(C(=O)c2cc(F)cc(B(O)O)c2)c1)NC(C)=O]

wherein each of the amino acid residues independently represents a D- or an L-amino acid form, and wherein * represents the point of attachment to said human insulin or human insulin analogue.

3. The compound according to any one of embodiments 1 to 2, wherein each of the modifying groups M is independently selected from the group of

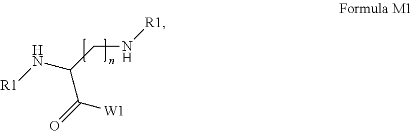

which represents a D- or an L-amino acid form, and

wherein n represents an integer in the range of 1 to 4;

wherein W1 is absent and represents the point of attachment * to said human insulin or human insulin analogue, or W1 represents
NH—CH₂—C(═O)—*,
NH—CH₂CH₂—C(═O)—*,
the D- or L-form of NH—CH(COOH)—CH₂CH₂—C(═O)—*,
the D- or L-form of NH—CH(COOH)—CH₂CH₂—C(═O)—NH—CH₂CH₂—C(═O)—*, or
NH—CH₂CH₂—C(═O)—NH—(CH₂)₂—O—(CH₂)₂—O—CH₂—CO—*,

wherein * represents the point of attachment to said human insulin or human insulin analogue; and

wherein R1 is selected from

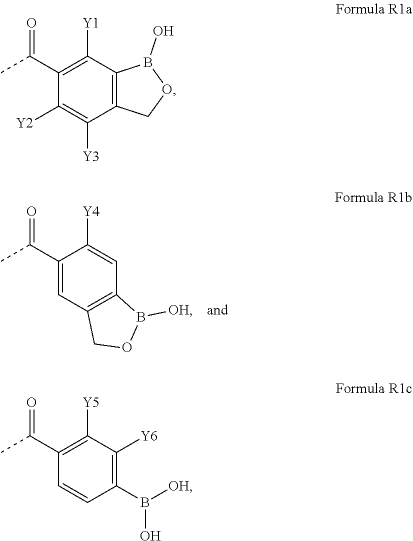

wherein Y1 and Y2 is H, and Y3 is F or CF₃; Y4 is H or F; and Y5 is H and Y6 is F;

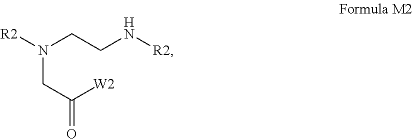

wherein W2 is absent and represents the point of attachment * to said human insulin or human insulin analogue, or W2 represents the D- or L-form of NH—CH(COOH)—CH₂CH₂—C(═O)—*, or NH—CH₂CH₂CH₂—C(═O)—*, wherein * represents the point of attachment to said human insulin or human insulin analogue; and

wherein R2 is selected from

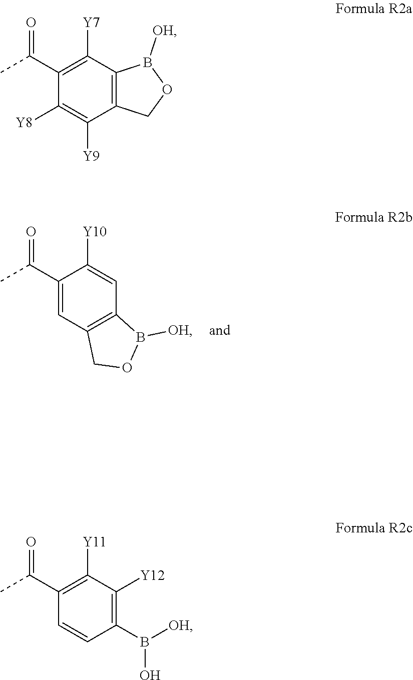

wherein Y7 is H; Y8 is H, Cl, CHF₂, or CF₃; Y9 is H, F, or CF₃; Y10 is F; Y11 is H; and Y12 is F; with the provisio that only one of Y8 and Y9 is H;

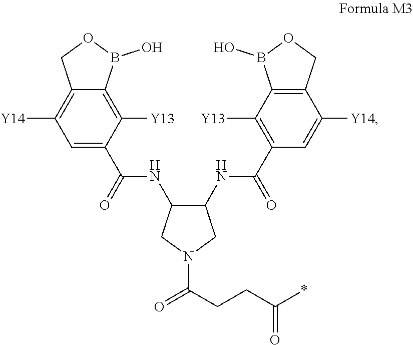

which represents a R,R or S,S or R,S stereoisomer of the 3,4-diamino-pyrrolidine; and

wherein * represents the point of attachment to said human insulin or human insulin analogue; and wherein Y13 is H or F; and Y14 is H or CF₃; with the provisio that only one of Y13 and Y14 is H;

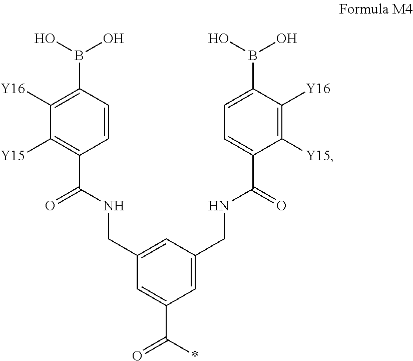

wherein * represents the point of attachment to said human insulin or human insulin analogue, and wherein Y15 and Y16 is independently selected from H, and F;

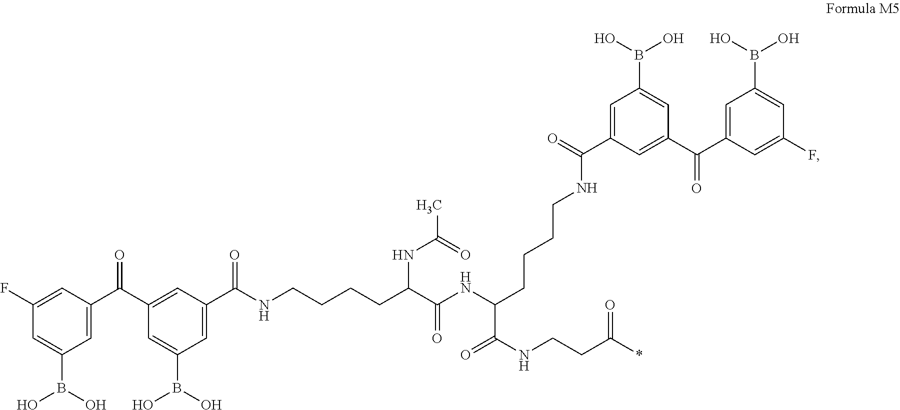 [SMILES: *C(=O)CCNC(=O)C(CCCCNC(=O)c1cc(B(O)O)cc(C(=O)c2cc(F)cc(B(O)O)c2)c1)NC(=O)C(CCCCNC(=O)c1cc(B(O)O)cc(C(=O)c2cc(F)cc(B(O)O)c2)c1)NC(C)=O]

which represents D- or L-amino acid forms, and wherein * represents the point of attachment to said human insulin or human insulin analogue;

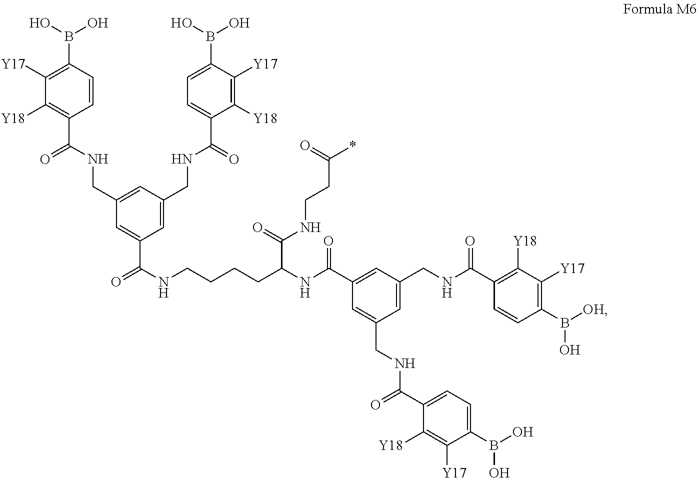

wherein the α-amino acid residue represents a D- or an L-amino acid form, and wherein * represents the point of attachment to said human insulin or human insulin analogue; and

wherein Y17 is H or F; and Y18 is H or F;

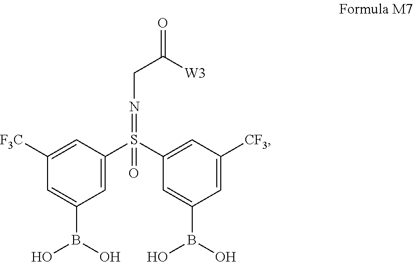

wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue;

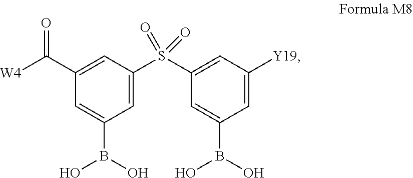

wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is CF₃ or SF₅;

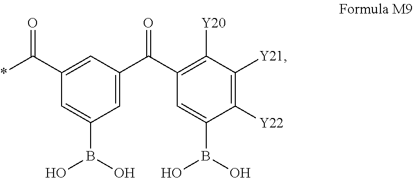

wherein * represents the point of attachment to said human insulin or human insulin analogue; and wherein each of Y20, Y21, and Y22 is independently selected from H, and F;

with the provisio that when Y21 is F, then Y20 and Y22 are H; and when Y21 is H, then Y20 and Y22 are F;

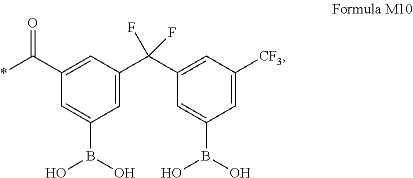 [SMILES: *C(=O)c1cc(B(O)O)cc(C(F)(F)c2cc(B(O)O)cc(C(F)(F)F)c2)c1]

wherein * represents the point of attachment to said human insulin or human insulin analogue; and

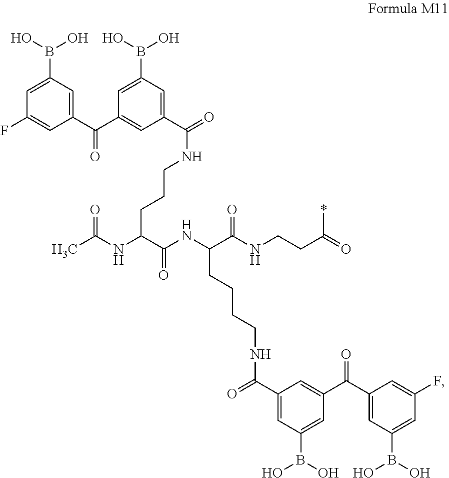 [SMILES: *C(=O)CCNC(=O)C(CCCCNC(=O)c1cc(B(O)O)cc(C(=O)c2cc(F)cc(B(O)O)c2)c1)NC(=O)C(CCCNC(=O)c1cc(B(O)O)cc(C(=O)c2cc(F)cc(B(O)O)c2)c1)NC(C)=O]

wherein each of the α-amino acid residues independently represents a D- or an L-amino acid form, and wherein * represents the point of attachment to said human insulin or human insulin analogue.

4. The compound according to any one of embodiments 1 to 3, wherein each of the modifying groups is independently selected from the group of

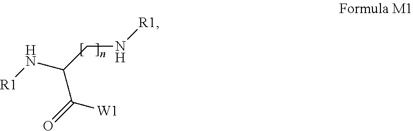

which represents a D- or an L-amino acid form, and wherein n is 1;

W1 represents NH—CH₂CH₂—C(═O)—* or the D- or L-form of NH—CH(COOH)—CH₂CH₂—C(═O)—*, wherein * represents the point of attachment to said human insulin or human insulin analogue; and

R1 is of

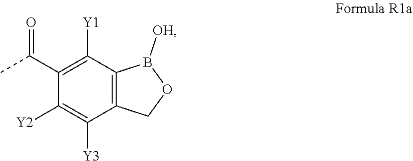

wherein Y1 and Y2 are H; and Y3 is F or CF₃;

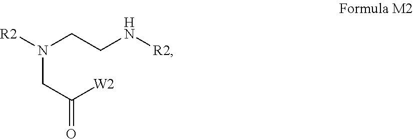

wherein W2 is absent and represents the point of attachment * to said human insulin or human insulin analogue, or W2 represents the D- or L-form of NH—CH(COOH)—CH₂CH₂—C(═O)—*, wherein * represents the point of attachment to said human insulin or human insulin analogue; and

wherein R2 is of

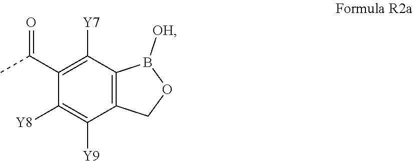

wherein Y7 and Y8 are H; and Y9 is CI, CHF₂, or CF₃;

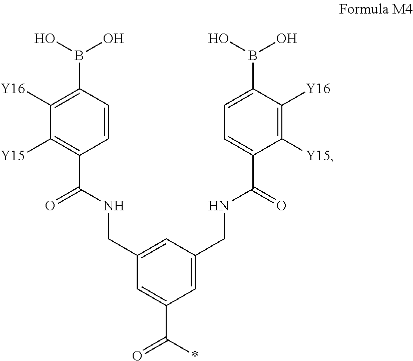

wherein * represents the point of attachment to said human insulin or human insulin analogue, and wherein Y15 is H, and Y16 is F;

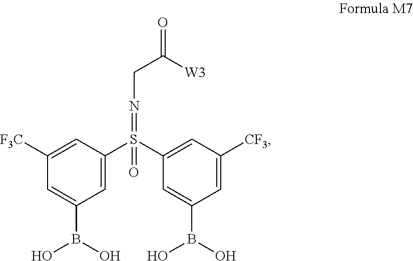

wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue;

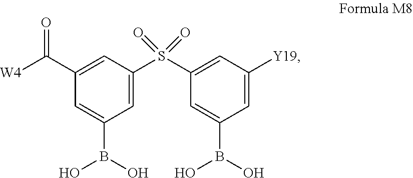

wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is CF₃; and

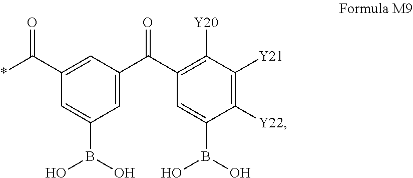

wherein * represents the point of attachment to said human insulin or human insulin analogue; and wherein each of Y20, Y21, and Y22 is independently selected from H and F;

with the provisio that when Y21 is F, then Y20 and Y22 are H; and when Y21 is H, then Y20 and Y22 are F.

5. The compound according to any one of embodiments 1 to 4, wherein the modifying groups are identical.

6. The compound according to any one of embodiments 1 to 5, wherein said human insulin or human insulin analogue optionally comprises a spacer selected from the group of
a) a peptide spacer at the C-terminal of the A-chain of said human insulin or human insulin analogue, wherein said peptide spacer comprises (GES)ₚK, wherein p is an integer from 3 to 12; or
b) a peptide spacer or a linker L at the N-terminal of the B-chain of said human insulin or human insulin analogue;
   wherein said peptide spacer comprises GKPG, GKP(G₄S)_{q}, KP(G₄S)ᵣ, GKPRGFFYTP(G₄S)ₛ, or TYFFGRKPD(G₄S)ₜ, wherein each of q, r, s and t is independently selected from an integer from 1 to 5; and

wherein said linker L is selected from

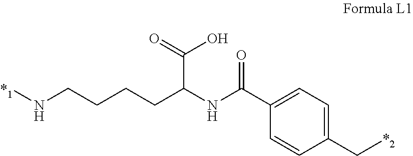

wherein *1 denotes the attachment point to the modifying group M and *2 denotes the attachment point to the amino group of the amino acid residue at the N-terminal of the B-chain of said human insulin or human insulin analogue;

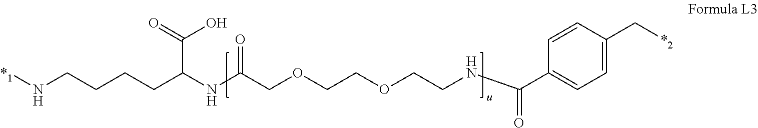

wherein *1 denotes the attachment point to the modifying group M and *2 denotes the attachment point to the amino group of the amino acid residue at N-terminal of the B-chain of said human insulin or human insulin analogue, and wherein u is 1, 2 or 3; and

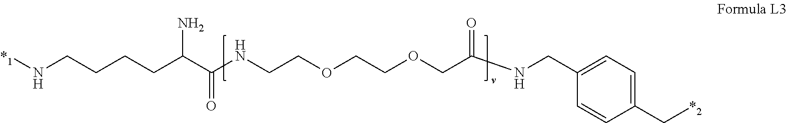

wherein *1 denotes the attachment point to the modifying group M and *2 denotes the attachment point to the amino group of the amino acid residue at N-terminal of the B-chain of said human insulin or human insulin analogue, and wherein v is 2 or 3.

7. The compound according to embodiment 6, wherein q is an integer selected from 1 to 3; r is 3; s is 2; and t is 3.

8. The compound according to any one of embodiments 1 to 7, wherein the chiral amino acids are in the L-form.

9. The compound according to any one of embodiments 1 to 8, wherein each modifying group M is attached to an attachment point selected from one of the following groups:
a) the amino group of the N-terminal amino acid residue of the A-chain of said human insulin or human insulin analogue;
b) the epsilon amino group of a lysine in position 22 of the A-chain of said human insulin analogue; or the epsilon amino group of the lysine in said optional peptide spacer at the C-terminal of the A-chain of said human insulin or human insulin analogue;
c) the amino group of the N-terminal amino acid residue of the B-chain of said human insulin or human insulin analogue;
   the epsilon amino group of a lysine residue in position 1 or position 4 of the B-chain of said human insulin analogue;
   the epsilon amino group of a lysine in said optional peptide spacer at the N-terminal of the B-chain of said human insulin or human insulin analogue; or
   the distal amino group marked with *1 in said optional linker L at the N-terminal of the B-chain of said human insulin or human insulin analogue; and
d) the epsilon amino group of a lysine in position 22 or position 29 of the B-chain of said human insulin or human insulin analogue.

10. The compound according to embodiment 9, wherein not more than one modifying group M is attached to a point of attachment within each of the groups a), b), c) and d).

11. The compound according to any one of embodiments 1 to 10, having exactly one, two, three or four modifying groups M.

12. The compound according to any one of embodiments 1 to 10, comprising at least two modifying groups M.

13. The compound according to any one of embodiments 1 to 10, having exactly two, three, or four modifying groups M.

14. The compound according to any one of embodiments 1 to 10, having exactly two modifying groups M.

15. The compound according to any one of embodiments 1 to 14, wherein said human insulin or human insulin analogue is a human insulin analogue comprising desB30.

16. The compound according to any one of embodiments 1 to 15, wherein said human insulin or human insulin analogue is a human insulin analogue selected from the group of desB30 human insulin (SEQ ID NO:1 and SEQ ID NO:11);

A21Q desB30 human insulin (SEQ ID NO:3 and SEQ ID NO:11);

A14E B25H desB30 human insulin (SEQ ID NO:4 and SEQ ID NO:12);

A14E B1K B2P B25H desB27 desB30 human insulin (SEQ ID NO:4 and SEQ ID NO:13);

A14E A22K B25H desB27 desB30 human insulin (SEQ ID NO:5 and SEQ ID NO:14);

A14E A22K B25H B27P B28G desB30 human insulin (SEQ ID NO:5 and SEQ ID NO:15);

A14E desB1-B2 B4K B5P desB30 human insulin (SEQ ID NO:4 and SEQ ID NO:16);

A14E desB1-B2 B3G B4K B5P desB30 human insulin (SEQ ID NO:4 and SEQ ID NO:17);

A14E B-1G B1K B2P desB30 human insulin (SEQ ID NO:4 and SEQ ID NO:18);

A22K desB30 human insulin (SEQ ID NO:6 and SEQ ID NO:11);

A22K B29R desB30 human insulin (SEQ ID NO:6 and SEQ ID NO:19);

A22K B22K B29R desB30 human insulin (SEQ ID NO:6 and SEQ ID NO:20); and

A-2K A-1P desB30 human insulin (SEQ ID NO:7 and SEQ ID NO:11).

17. A compound according to embodiment 1, comprising

i) human insulin or a human insulin analogue, wherein said human insulin or human insulin analogue optionally comprises a spacer selected from a peptide spacer or a linker L at the N-terminal of the B-chain of said human insulin or human insulin analogue;
wherein said peptide spacer comprises GKPG, GKP(G₄S)_{q}, KP(G₄S)ᵣ, GKPRGFFYTP(G₄S)ₛ, or TYFFGRKPD(G₄S)ₜ, wherein each of q, r, s and t is independently selected from an integer from 1 to 5; and
wherein said linker L is selected from

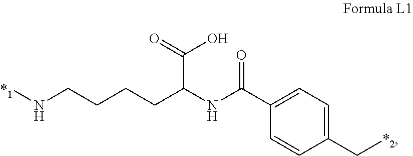

wherein *1 denotes the attachment point to the modifying group M and *2 denotes the attachment point to the amino group of the amino acid residue at the N-terminal of the B-chain of said human insulin or human insulin analogue;

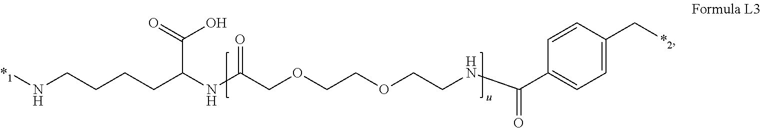

wherein *1 denotes the attachment point to the modifying group M and *2 denotes the attachment point to the amino group of the amino acid residue at N-terminal of the B-chain of said human insulin or human insulin analogue, and wherein u is 1, 2 or 3; and

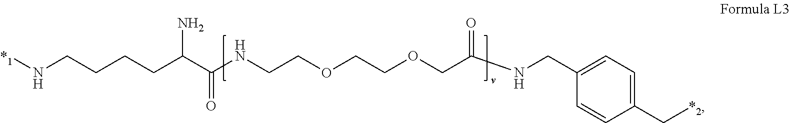

wherein *1 denotes the attachment point to the modifying group M and *2 denotes the attachment point to the amino group of the amino acid residue at N-terminal of the B-chain of said human insulin or human insulin analogue, and wherein v is 2 or 3;

ii) two, three or four modifying groups M, wherein each of the modifying groups M is independently selected from the group of

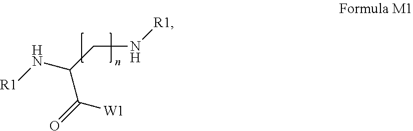

which represents a D- or an L-amino acid form, and

wherein n represents an integer in the range of 1 to 4;

wherein W1 is absent and represents the point of attachment * to said human insulin or human insulin analogue, or W1 represents
NH—CH₂CH₂—C(═O)—*,
the D- or L-form of NH—CH(COOH)—CH₂CH₂—C(═O)—*,

wherein * represents the point of attachment to said human insulin or human insulin analogue; and

wherein R1 is selected from

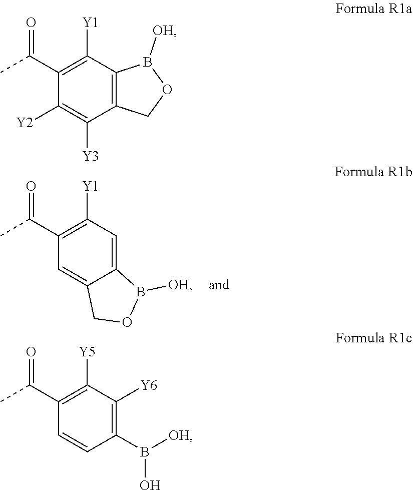

wherein Y1 and Y2 is H, and Y3 is F or CF₃; Y4 is F; and Y5 is H and Y6 is F;

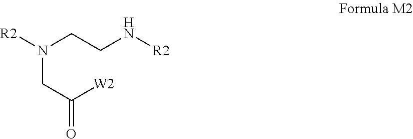

wherein W2 is absent and represents the point of attachment * to said human insulin or human insulin analogue, or W2 represents the D- or L-form of NH—CH(COOH)—CH₂CH₂—C(═O)—*, wherein * represents the point of attachment to said human insulin or human insulin analogue; and

wherein R2 is selected from

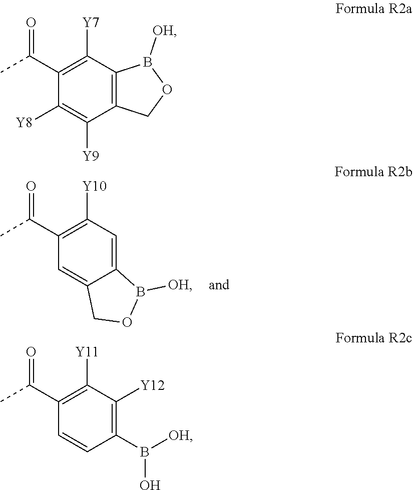

wherein Y7 is H; Y8 is H, Cl, CHF₂, or CF₃; Y9 is H, F, or CF₃; Y10 is F; Y11 is H; and Y12 is F; with the provisio that only one of Y8 and Y9 is H;

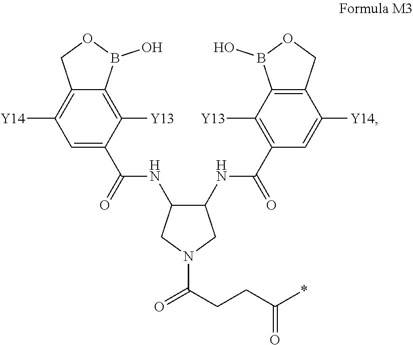

which represents a R,R or S,S or R,S or stereoisomer of the 3,4-diamino-pyrrolidine; and

wherein * represents the point of attachment to said human insulin or human insulin analogue; and wherein Y13 is H or F; and Y14 is H or CF₃; with the provisio that only one of Y13 and Y14 is H;

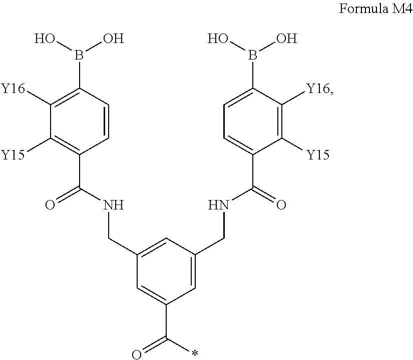

wherein * represents the point of attachment to said human insulin or human insulin analogue, and wherein Y15 is H and Y16 is F;

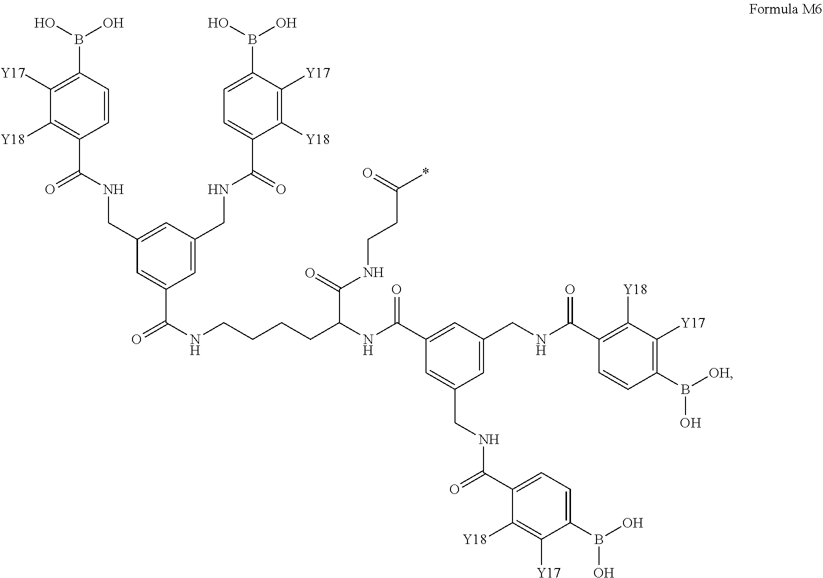

wherein the α-amino acid residue represents a D- or an L-amino acid form, and wherein * represents the point of attachment to said human insulin or human insulin analogue; and

wherein Y17 is F; and Y18 is H;

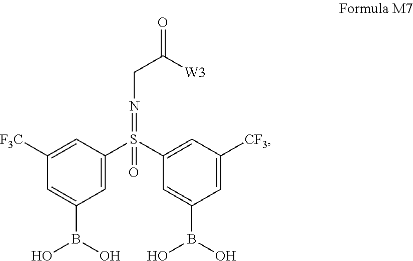

wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue;

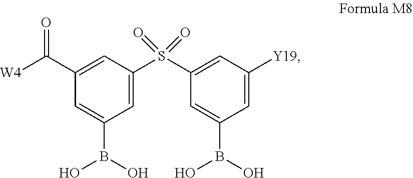

wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is CF₃ or SF₅;

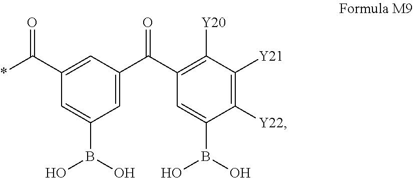

wherein * represents the point of attachment to said human insulin or human insulin analogue; and wherein each of Y20, Y21, and Y22 is independently selected from H, and F;

with the provisio that when Y21 is F, then Y20 and Y22 are H; and when Y21 is H, then Y20 and Y22 are F; and

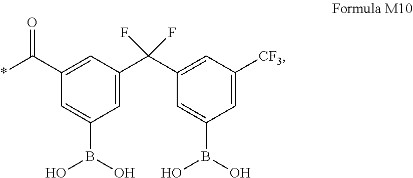 [SMILES: *C(=O)c1cc(B(O)O)cc(C(F)(F)c2cc(B(O)O)cc(C(F)(F)F)c2)c1]

wherein * represents the point of attachment to said human insulin or human insulin analogue; and

wherein each modifying group M is attached to an attachment point selected from one of the following groups:
a) the amino group of the N-terminal amino acid residue of the A-chain of said human insulin or human insulin analogue;
b) the epsilon amino group of a lysine in position 22 of the A-chain of said human insulin analogue; or the epsilon amino group of the lysine in said optional peptide spacer at the C-terminal of the A-chain of said human insulin or human insulin analogue;
c) the amino group of the N-terminal amino acid residue of the B-chain of said human insulin or human insulin analogue;
   the epsilon amino group of a lysine residue in position 1 or position 4 of the B-chain of said human insulin analogue;
   the epsilon amino group of a lysine in said optional peptide spacer at the N-terminal of the B-chain of said human insulin or human insulin analogue; or
   the distal amino group marked with *1 in said optional linker L at the N-terminal of the B-chain of said human insulin or human insulin analogue; and
d) the epsilon amino group of a lysine in position 22 or position 29 of the B-chain of said human insulin or human insulin analogue,

wherein one modifying group M is attached to one of the attachment points c) and one modifying group M is attached to the attachment point d).

18. A compound according to embodiment 17, wherein not more than one modifying group M is attached to a point of attachment within each of the groups a), b), c) and d).

19. A compound according to any one of embodiments 17 to 18, wherein the compound has exactly two modifying groups M, wherein one modifying group M is attached to the epsilon amino group of a lysine in position 22 or position 29 of the B-chain of said human insulin or human insulin analogue; and one modifying group M is attached to the amino group of the N-terminal amino acid residue of the B-chain of said human insulin or human insulin analogue;
the epsilon amino group of a lysine residue in position 1 or position 4 of the B-chain of said human insulin analogue;
the epsilon amino group of a lysine in said optional peptide spacer at the N-terminal of the B-chain of said human insulin or human insulin analogue; or
the distal amino group marked with *1 in said optional linker L at the N-terminal of the B-chain of said human insulin or human insulin analogue.

20. A compound according to any one of embodiments 17 to 19, comprising

i) human insulin or a human insulin analogue, wherein said human insulin or human insulin analogue optionally comprises a peptide spacer at the N-terminal of the B-chain of said human insulin or human insulin analogue;
wherein said peptide spacer comprises GKPG, GKP(G₄S)_{q}, KP(G₄S)ᵣ, GKPRGFFYTP(G₄S)ₛ, or TYFFGRKPD(G₄S)ₜ, wherein q is an integer from 1 to 3; r is 3; s is 2 and t is 3;

ii) two modifying groups M, wherein each of the modifying groups M is independently selected from the group of

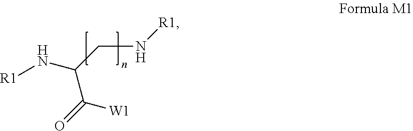

which represents a D- or an L-amino acid form, and wherein n is 1; W1 represents NH—CH₂CH₂—C(═O)—*, or the D- or L-form of NH—CH(COOH)—CH₂CH₂—C(═O)—*, wherein * represents the point of attachment to said human insulin or human insulin analogue; and

wherein R1 is of

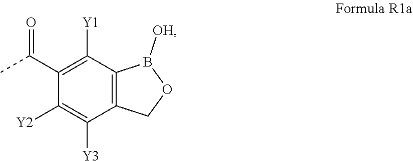

wherein Y1 and Y2 is H, and Y3 is CF₃;

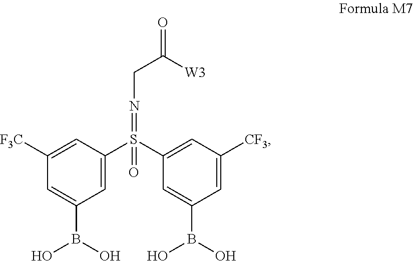

wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue;

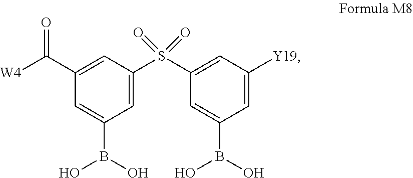

wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is CF₃;

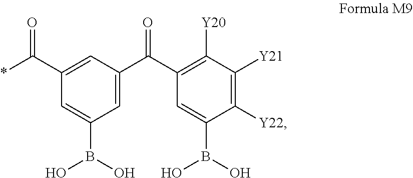

wherein * represents the point of attachment to said human insulin or human insulin analogue; and wherein each of Y20, Y21, and Y22 is independently selected from H, and F; with the provisio that when Y21 is F, then Y20 and Y22 are H; and when Y21 is H, then Y20 and Y22 are F; and

wherein one modifying group M is attached to the epsilon amino group of a lysine in position 29 of the B-chain of said human insulin or human insulin analogue; and one modifying group M is attached to
the epsilon amino group of a lysine residue in position 1 or position 4 of the B-chain of said human insulin analogue; or
the epsilon amino group of a lysine in said optional peptide spacer at the N-terminal of the B-chain of said human insulin or human insulin analogue.

21. The compound according to any one of embodiments 17 to 20, comprising

i) a human insulin analogue, wherein said human insulin analogue comprises a peptide spacer at the N-terminal of the B-chain of said human insulin or human insulin analogue; wherein said peptide spacer comprises GKP(G₄S)_{q}, or KP(G₄S)ᵣ, wherein q is an integer from 1 to 3; and r is 3;

ii) two modifying groups M, independently selected from the group of

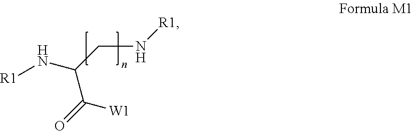

which represents a D- or an L-amino acid form, and wherein n is 1; W1 represents NH—CH₂CH₂—C(═O)—*, or the D- or L-form of NH—CH(COOH)—CH₂CH₂—C(═O)—*, wherein * represents the point of attachment to said human insulin or human insulin analogue; and

wherein R1 is of

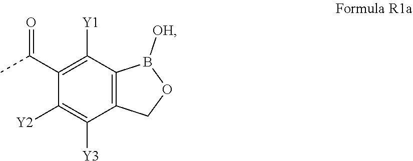

wherein Y1 and Y2 is H, and Y3 is CF₃;

wherein one modifying group M is attached to the epsilon amino group of the lysine in said peptide spacer; and one modifying group M is attached to the epsilon amino group of a lysine in position 29 of the B-chain of said human insulin or human insulin analogue.

22. A compound according to any one of embodiments 17-21, consisting of

i) a human insulin analogue, wherein said human insulin analogue optionally comprises a peptide spacer at the N-terminal of the B-chain of said human insulin or human insulin analogue;
wherein said peptide spacer comprises GKPG, GKP(G₄S)_{q}, KP(G₄S)ᵣ, GKPRGFFYTP(G₄S)ₛ, or TYFFGRKPD(G₄S)ₜ, wherein q is an integer from 1 to 3; r is 3; s is 2 and t is 3;

ii) two modifying groups, wherein each of the modifying groups M is independently selected from the group of

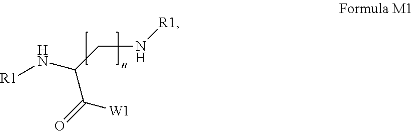

which represents a D- or an L-amino acid form, and wherein n is 1; W1 represents NH—CH₂CH₂—C(═O)—*, or the D- or L-form of NH—CH(COOH)—CH₂CH₂—C(═O)—*, wherein * represents the point of attachment to said human insulin or human insulin analogue; and

wherein R1 is of

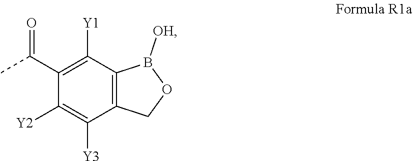

wherein Y1 and Y2 is H, and Y3 is CF₃;

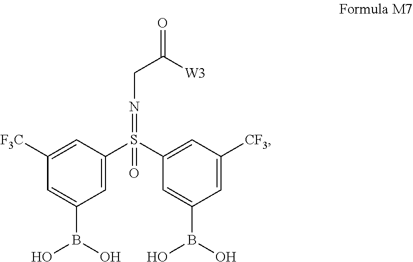

wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue;

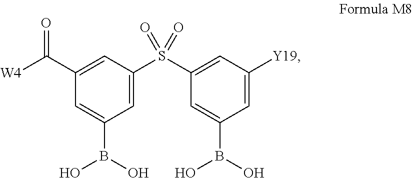

wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is CF₃;

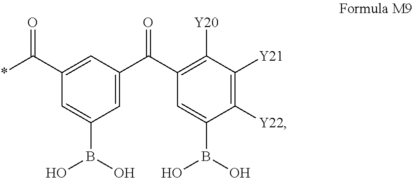

wherein * represents the point of attachment to said human insulin or human insulin analogue; and wherein each of Y20, Y21, and Y22 is independently selected from H, and F;

with the provisio that when Y21 is F, then Y20 and Y22 are H; and when Y21 is H, then Y20 and Y22 are F; and

wherein one modifying group M is attached to the epsilon amino group of a lysine in position 29 of the B-chain of said human insulin or human insulin analogue; and

one modifying group M is attached to:
the epsilon amino group of a lysine residue in position 1 or position 4 of the B-chain of said human insulin analogue, or
the epsilon amino group of a lysine in said optional peptide spacer at the N-terminal of the B-chain of said human insulin or human insulin analogue.

23. The compound according to any one of embodiments 17 to 22, consisting of

i) a human insulin analogue, wherein said human insulin analogue has a peptide spacer at the N-terminal of the B-chain of said human insulin or human insulin analogue; wherein said peptide spacer is GKP(G₄S)_{q}, or KP(G₄S)ᵣ, wherein q is an integer from 1 to 3; and r is 3;

ii) two modifying groups M, independently selected from the group of

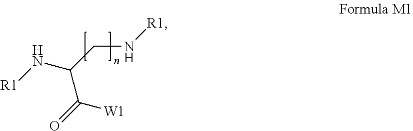

which represents a D- or an L-amino acid form, and wherein n is 1; W1 represents NH—CH₂CH₂—C(═O)—*, or the D- or L-form of NH—CH(COOH)—CH₂CH₂—C(═O)—*, wherein * represents the point of attachment to said human insulin or human insulin analogue; and

wherein R1 is of

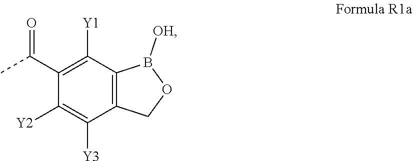

wherein Y1 and Y2 is H, and Y3 is CF₃;

wherein one modifying group M is attached to the epsilon amino group of the lysine in said peptide spacer; and one modifying group M is attached to the epsilon amino group of a lysine in position 29 of the B-chain of said human insulin or human insulin analogue.

24. The compound according to any one of embodiments 17 to 23, wherein the chiral amino acids are in the L-form.

25. The compound according to any one of embodiments 17 to 24, wherein the compound has exactly 2 modifying groups M.

26. The compound according to any one of embodiments 17 to 25, wherein the modifying groups M are identical.

27. The compound according to any one of embodiments 17 to 26, wherein said human insulin analogue comprises desB30.

28. The compound according to any one of embodiments 17 to 27, wherein said human insulin or human insulin analogue is a human insulin analogue selected from the group of desB30 human insulin (SEQ ID NO:1 and SEQ ID NO:11);

A14E B25H desB30 human insulin (SEQ ID NO:4 and SEQ ID NO:12);

A14E B1K B2P B25H desB27 desB30 human insulin (SEQ ID NO:4 and SEQ ID NO:13);

A14E desB1-B2 B4K B5P desB30 human insulin (SEQ ID NO:4 and SEQ ID NO:16);

A14E desB1-B2 B3G B4K B5P desB30 human insulin (SEQ ID NO:4 and SEQ ID NO:17);

A14E B-1G B1K B2P desB30 human insulin (SEQ ID NO:4 and SEQ ID NO:18);

A22K desB30 human insulin (SEQ ID NO:6 and SEQ ID NO:11);

A22K B29R desB30 human insulin (SEQ ID NO:6 and SEQ ID NO:19); and

A22K B22K B29R desB30 human insulin (SEQ ID NO:6 and SEQ ID NO:20).

29. The compound according to any one of embodiments 17 to 28, wherein said human insulin analogue comprising said spacer is selected from the group of

B1-KPGGGGSGGGGSGGGGS desB30 human insulin (SEQ ID NO:1 and SEQ ID NO:21);

B1-KPGGGGSGGGGSGGGGS A14E B25H desB30 human insulin (SEQ ID NO:4 and SEQ ID NO:22);

B1-GKPGGGGSGGGGSGGGGS desB30 human insulin (SEQ ID NO:1 and SEQ ID NO:23);

B1-GKPGGGGSGGGGS desB30 human insulin (SEQ ID NO:1 and SEQ ID NO:24); and

B1-GKPGGGGS desB30 human insulin (SEQ ID NO:1 and SEQ ID NO:25).

30. The compound according to any one of embodiments 17 to 29, wherein the compound is selected from the group of:

The compound of Example 280; The compound of Example 284; The compound of Example 285; The compound of Example 288; The compound of Example 291; The compound of Example 300; The compound of Example 301; The compound of Example 324; The compound of Example 327; The compound of Example 331; The compound of Example 333; and the compound of Example 335.

31. The compound according to any one of embodiments 17 to 30, wherein the compound is selected from the group of:

The compound of Example 280; The compound of Example 285; The compound of Example 288; The compound of Example 291; The compound of Example 300; The compound of Example 301; The compound of Example 327; The compound of Example 331; The compound of Example 333; and the compound of Example 335.

32. The compound according to any one of embodiments 17 to 31, wherein the compound is the compound of Example 280.

33. The compound according to any one of embodiments 17 to 31, wherein the compound is the compound of Example 284.

34. The compound according to any one of embodiments 17 to 31, wherein the compound is the compound of Example 285.

35. The compound according to any one of embodiments 17 to 31, wherein the compound is the compound of Example 288.

36. The compound according to any one of embodiments 17 to 31, wherein the compound is the compound of Example 291.

37. The compound according to any one of embodiments 17 to 31, wherein the compound is the compound of Example 300.

38. The compound according to any one of embodiments 17 to 31, wherein the compound is the compound of Example 301.

39. The compound according to any one of embodiments 17 to 31, wherein the compound is the compound of Example 324.

40. The compound according to any one of embodiments 17 to 31, wherein the compound is the compound of Example 327.

41. The compound according to any one of embodiments 17 to 31, wherein the compound is the compound of Example 331.

42. The compound according to any one of embodiments 17 to 31, wherein the compound is the compound of Example 333.

43. The compound according to to any one of embodiments 17 to 31, wherein the compound is the compound of Example 335.

44. A compound according to embodiment 1, comprising

i) human insulin or a human insulin analogue;

ii) two modifying groups M, independently selected from the group of

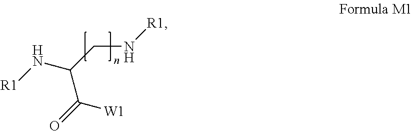

which represents a D- or an L-amino acid form, and

wherein n represents an integer in the range of 1 to 4;

wherein W1 is absent and represents the point of attachment * to said human insulin or human insulin analogue, or W1 represents
NH—CH₂—C(═O)—*,
NH—CH₂CH₂—C(═O)—*,

wherein * represents the point of attachment to said human insulin or human insulin analogue; and

wherein R1 is selected from

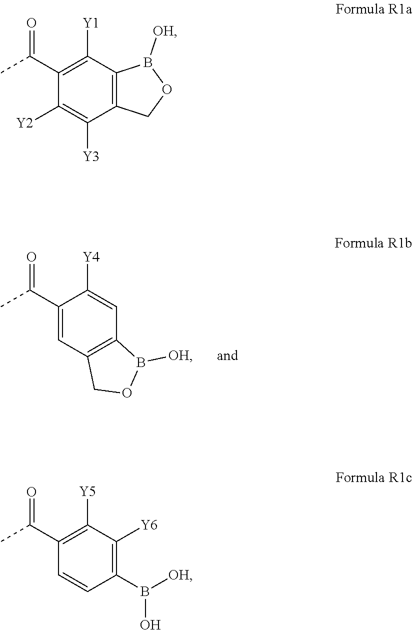

wherein Y1 and Y2 is H, and Y3 is F or CF₃; Y4 is H or F; and Y5 is H and Y6 is F;

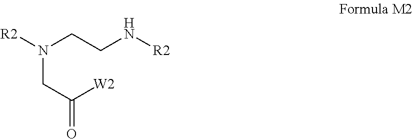

wherein W2 is absent and represents the point of attachment * to said human insulin or human insulin analogue; and

wherein R2 is selected from

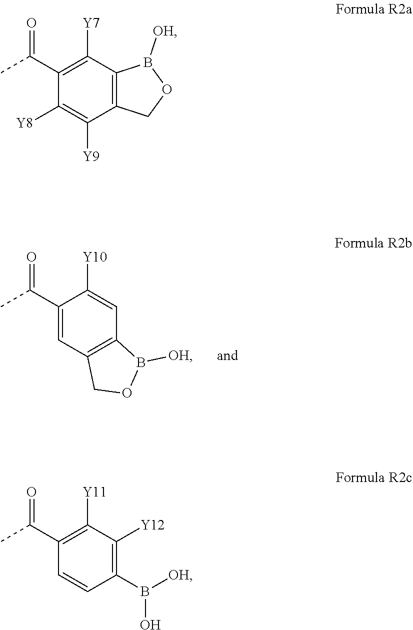

wherein Y7 is H; Y8 is H, Cl, CHF₂, or CF₃; Y9 is H, F, or CF₃; Y10 is F; Y11 is H; and Y12 is F; with the provisio that only one of Y8 and Y9 is H;

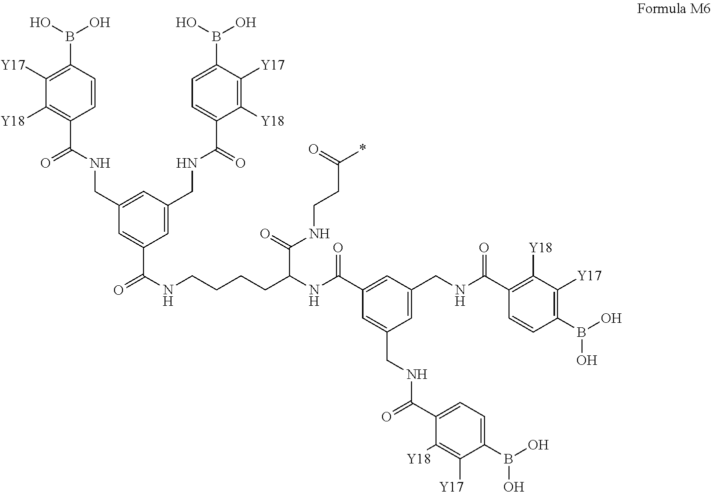

wherein the α-amino acid residue represents a D- or an L-amino acid form, and wherein * represents the point of attachment to said human insulin or human insulin analogue; and

wherein Y17 is H or F; and Y18 is H or F; and

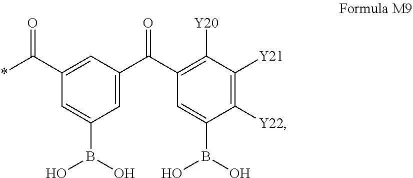

wherein * represents the point of attachment to said human insulin or human insulin analogue; and wherein each of Y20, Y21, and Y22 is independently selected from H, and F; with the provisio that when Y21 is F, then Y20 and Y22 are H; and when Y21 is H, then Y20 and Y22 are F; and

wherein one modifying group M is attached to the amino group of the N-terminal amino acid residue of the A-chain of said human insulin or human insulin analogue; and one modifying group M is attached to the epsilon amino group of a lysine in position 29 of the B-chain of said human insulin or human insulin analogue.

45. The compound according to embodiment 44, wherein the modifying groups M are identical.

46. The compound according to any one of embodiments 44 to 45, wherein said human insulin or human insulin analogue is a human insulin analogue comprising desB30.

47. The compound according to embodiment 46, wherein said human insulin analogue is desB30 human insulin.

48. A compound according to embodiment 1, comprising

i) human insulin or a human insulin analogue, wherein said human insulin or human insulin analogue optionally comprises a peptide spacer at the C-terminal of the A-chain of said human insulin or human insulin analogue, wherein said peptide spacer comprises (GES)ₚK, wherein p is an integer from 3 to 12;

ii) two modifying groups M, independently selected from the group of

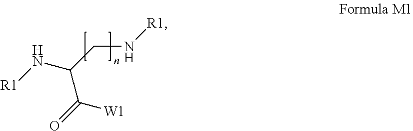

which represents a D- or an L-amino acid form, and

wherein n represents an integer in the range of 1 to 4;

wherein W1 is absent and represents the point of attachment * to said human insulin or human insulin analogue, or W1 represents
NH—CH₂—C(═O)—*,
NH—CH₂CH₂—C(═O)—*,
the D- or L-form of NH—CH(COOH)—CH₂CH₂—C(═O)—*,
the D- or L-form of NH—CH(COOH)—CH₂CH₂—C(═O)—NH—CH₂CH₂—C(═O)—*, or NH—CH₂CH₂—C(═O)—NH—(CH₂)₂—O—(CH₂)₂—O—CH₂—CO—*,

wherein * represents the point of attachment to said human insulin or human insulin analogue; and

wherein R1 is selected from

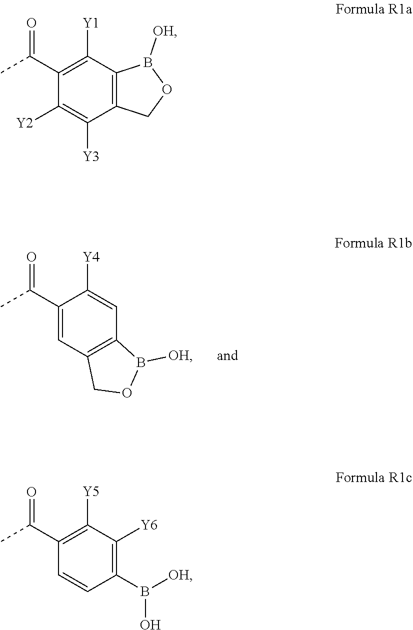

wherein Y1 and Y2 is H, and Y3 is F or CF₃; Y4 is H or F; and Y5 is H and Y6 is F;

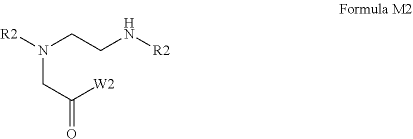

wherein W2 is absent and represents the point of attachment * to said human insulin or human insulin analogue, or W2 represents the D- or L-form of NH—CH(COOH)—CH₂CH₂—C(═O)—*, or NH—CH₂CH₂CH₂—C(═O)—*, wherein * represents the point of attachment to said human insulin or human insulin analogue; and

wherein R2 is selected from

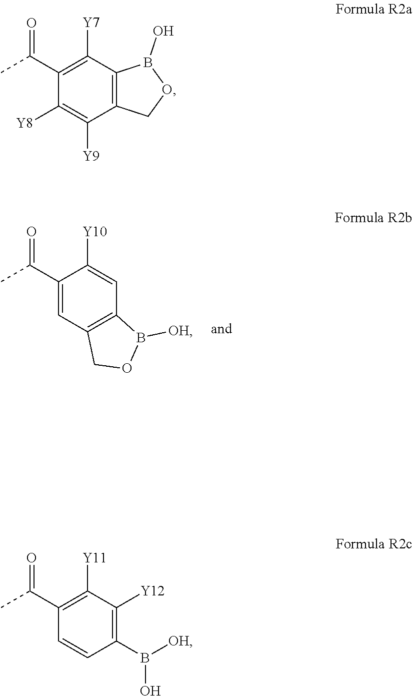

wherein Y7 is H; Y8 is H, Cl, CHF₂, or CF₃; Y9 is H, F, or CF₃; Y10 is F; Y11 is H; and Y12 is F; with the provisio that only one of Y8 and Y9 is H;

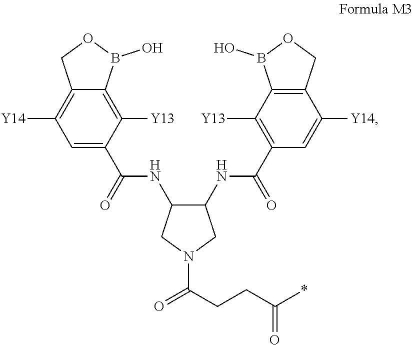

which represents a R,R or S,S, or R,S stereoisomer of the 3,4-diamino-pyrrolidine; and

wherein * represents the point of attachment to said human insulin or human insulin analogue; and wherein Y13 is H or F; and Y14 is H or CF₃; with the provisio that only one of Y13 and Y14 is H;

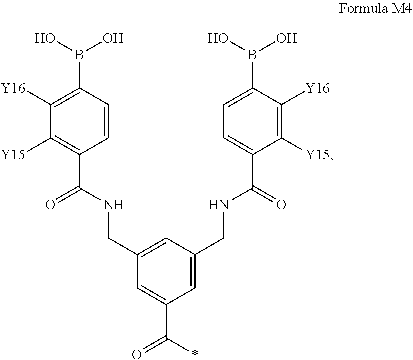

wherein * represents the point of attachment to said human insulin or human insulin analogue, and wherein Y15 and Y16 is independently selected from H, and F;

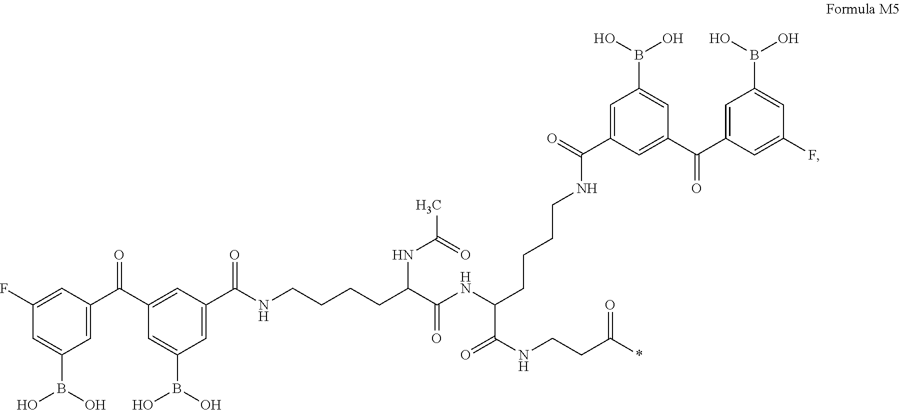 [SMILES: *C(=O)CCNC(=O)C(CCCCNC(=O)c1cc(B(O)O)cc(C(=O)c2cc(F)cc(B(O)O)c2)c1)NC(=O)C(CCCCNC(=O)c1cc(B(O)O)cc(C(=O)c2cc(F)cc(B(O)O)c2)c1)NC(C)=O]

wherein each of the amino acid residues represents a D- or an L-amino acid form, and

wherein * represents the point of attachment to said human insulin or human insulin analogue;

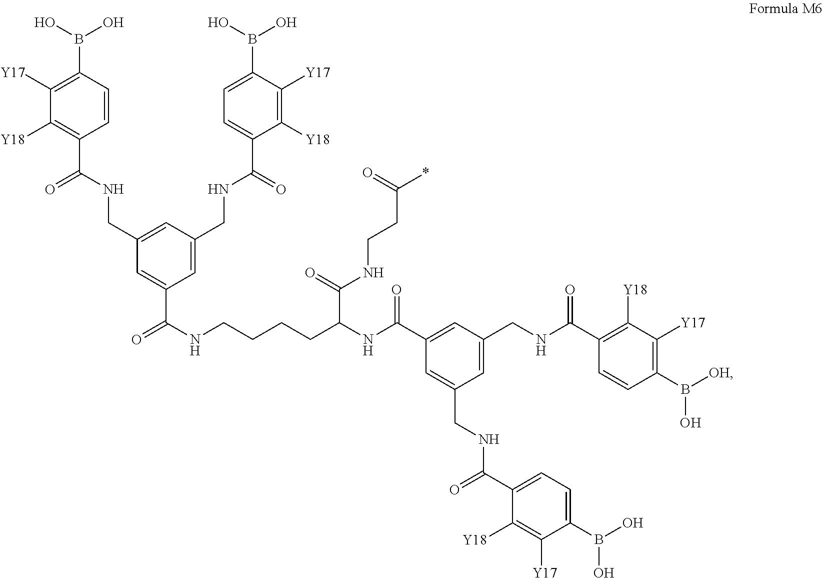

wherein the α-amino acid residue represents a D- or an L-amino acid form, and wherein * represents the point of attachment to said human insulin or human insulin analogue; and

wherein Y17 is H or F; and Y18 is H or F;

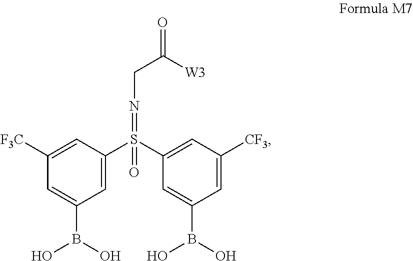

wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue;

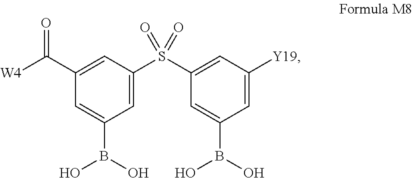

wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is CF₃ or SF₅;

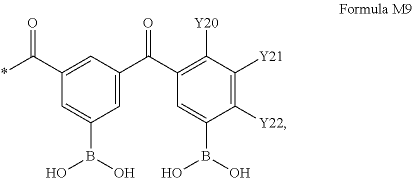

wherein * represents the point of attachment to said human insulin or human insulin analogue; and wherein each of Y20, Y21, and Y22 is independently selected from H, and F;

with the provisio that when Y21 is F, then Y20 and Y22 are H; and when Y21 is H, then Y20 and Y22 are F;

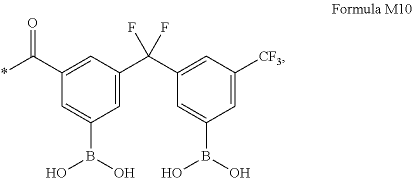 [SMILES: *C(=O)c1cc(B(O)O)cc(C(F)(F)c2cc(B(O)O)cc(C(F)(F)F)c2)c1]

wherein * represents the point of attachment to said human insulin or human insulin analogue; and

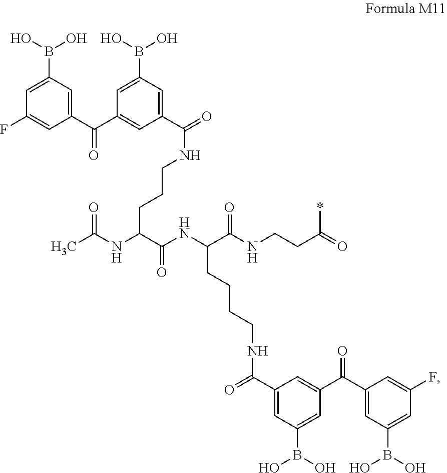 [SMILES: *C(=O)CCNC(=O)C(CCCCNC(=O)c1cc(B(O)O)cc(C(=O)c2cc(F)cc(B(O)O)c2)c1)NC(=O)C(CCCNC(=O)c1cc(B(O)O)cc(C(=O)c2cc(F)cc(B(O)O)c2)c1)NC(C)=O]

wherein each of the amino acid residues represents a D- or an L-amino acid form, and

wherein * represents the point of attachment to said human insulin or human insulin analogue; and

wherein one modifying group M is attached to the epsilon amino group of a lysine in position 22 or position 29 of the B-chain of said human insulin or human insulin analogue; and one modifying group M is attached to:
the epsilon amino group of a lysine in position 22 of the A-chain of said human insulin analogue; or
the epsilon amino group of the lysine in said optional peptide spacer at the C-terminal of the A-chain of said human insulin or human insulin analogue.

49. The compound according to embodiment 48, wherein the chiral amino acids are in the L-form.

50. The compound according to any one of embodiments 48 to 49, wherein the modifying groups M are identical.

51. The compound according to any one of embodiments 48 to 51, wherein said human insulin or human insulin analogue is a human insulin analogue comprising desB30.

52. The compound according to any one of embodiments 48 to 51, wherein said human insulin or human insulin analogue is selected from the group of:

A21Q desB30 human insulin (SEQ ID NO:3 and SEQ ID NO:11);

A14E A22K B25H desB27 desB30 human insulin (SEQ ID NO:5 and SEQ ID NO:14);

A14E A22K B25H B27P B28G desB30 human insulin (SEQ ID NO:5 and SEQ ID NO:15);

A22K desB30 human insulin (SEQ ID NO:6 and SEQ ID NO:11); and

A22K B22K B29R desB30 human insulin (SEQ ID NO:6 and SEQ ID NO:20).

53. The compound according to any one of embodiments 48 to 52, wherein the compound is selected from the group of:

The compound of Example 227; The compound of Example 239; The compound of Example 240; The compound of Example 241; and The compound of Example 272.

54. A compound according to embodiment 1, comprising

i) human insulin or a human insulin analogue;

ii) one modifying group M, selected from the group of

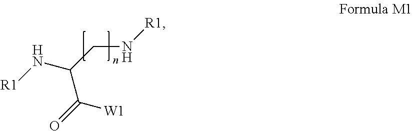

which represents a D- or an L-amino acid form, and

wherein n represents an integer in the range of 1 to 4;

wherein W1 is absent and represents the point of attachment * to said human insulin or human insulin analogue, or W1 represents
NH—CH₂—C(═O)—*,
NH—CH₂CH₂—C(═O)—*,
the D- or L-form of NH—CH(COOH)—CH₂CH₂—C(═O)—*,
the D- or L-form of NH—CH(COOH)—CH₂CH₂—C(═O)—NH—CH₂CH₂—C(═O)—*, or
NH—CH₂CH₂—C(═O)—NH—(CH₂)₂—O—(CH₂)₂—O—CH₂—CO—*,

wherein * represents the point of attachment to said human insulin or human insulin analogue; and

wherein R1 is selected from

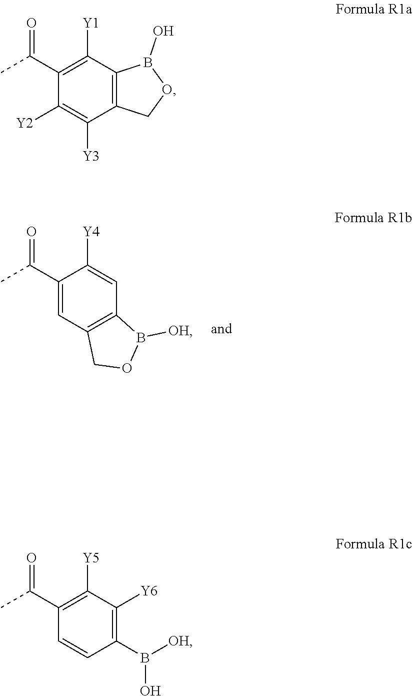

wherein Y1 and Y2 is H, and Y3 is F or CF₃; Y4 is H or F; and Y5 is H and Y6 is F;

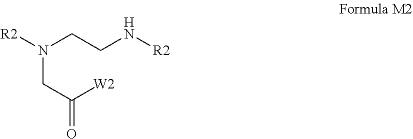

wherein W2 is absent and represents the point of attachment * to said human insulin or human insulin analogue, or W2 represents the D- or L-form of NH—CH(COOH)—CH₂CH₂—C(═O)—*, or NH—CH₂CH₂CH₂—C(═O)—*, wherein * represents the point of attachment to said human insulin or human insulin analogue; and

wherein R2 is selected from

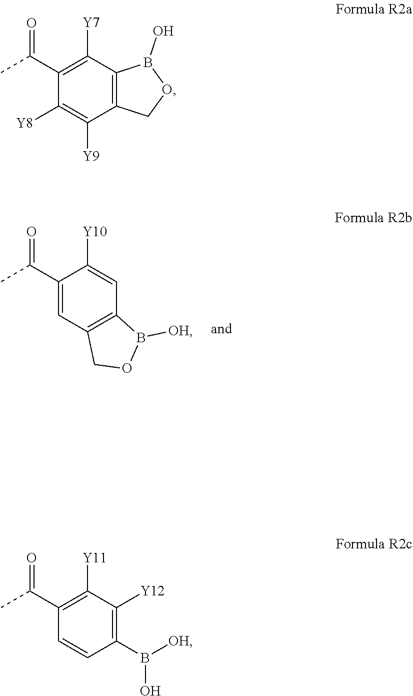

wherein Y7 is H; Y8 is H, Cl, CHF₂, or CF₃; Y9 is H, F, or CF₃; Y10 is F; Y11 is H; and Y12 is F; with the provisio that only one of Y8 and Y9 is H;

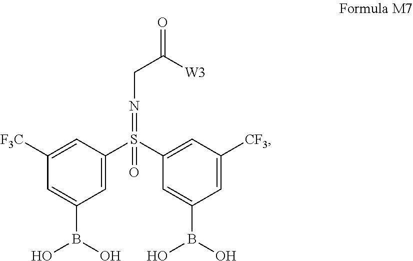

wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue;

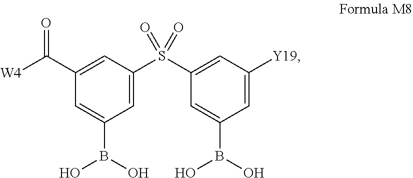

wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is CF₃ or SF₅;

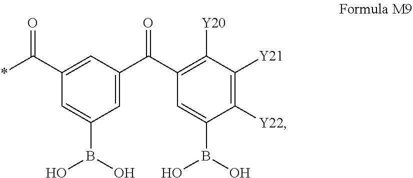

wherein * represents the point of attachment to said human insulin or human insulin analogue; and wherein each of Y20, Y21, and Y22 is independently selected from H, and F;

with the provisio that when Y21 is F, then Y20 and Y22 are H; and when Y21 is H, then Y20 and Y22 are F;

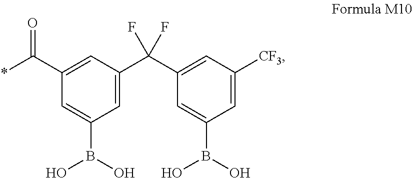 [SMILES: *C(=O)c1cc(B(O)O)cc(C(F)(F)c2cc(B(O)O)cc(C(F)(F)F)c2)c1]

wherein * represents the point of attachment to said human insulin or human insulin analogue; and

wherein the modifying group M is attached to the epsilon amino group of a lysine in position 22 of the A-chain of said human insulin analogue, or to the epsilon amino group of a lysine in position 22 or position 29 of the B-chain of said human insulin or human insulin analogue.

55. The compound according to embodiment 54, wherein said human insulin or human insulin analogue is a human insulin analogue comprising A22K and desB30.

56. A compound according to any one of embodiments 1 to 55, wherein the compound has the ability to bind to the insulin receptor.

57. A compound according to any one of embodiments 1 to 55, wherein the compound has higher insulin receptor affinity in presence of 20 mM glucose than when no glucose is present.

58. A compound according to any one of embodiments 1 to 55, wherein the compound has at least 3-fold higher insulin receptor affinity in presence of 20 mM glucose than when no glucose is present.

59. A compound according to any one of embodiments 1 to 55, wherein the compound has at least 10-fold higher insulin receptor affinity in presence of 20 mM glucose than when no glucose is present.

60. A compound according to any one of embodiments 1 to 55, wherein the compound has at least 15-fold higher insulin receptor affinity in presence of 20 mM glucose than when no glucose is present.

61. A composition comprising a compound according to any one of embodiments 1-55.

62. A compound according to any one of embodiments 1-55, for use as a medicament.

63. A compound according to any one of embodiments 1-55, for use in the prevention or treatment of diabetes, diabetes of Type 1, diabetes of Type 2, impaired glucose tolerance, hyperglycemia, and metabolic syndrome (metabolic syndrome X, insulin resistance syndrome).

64. Use of a compound according to any one of embodiments 1-55 or the composition according to embodiment 61, for the manufacture of a medicament for the treatment or prevention of diabetes, diabetes of Type 1, diabetes of Type 2, impaired glucose tolerance, hyperglycemia, and metabolic syndrome (metabolic syndrome X, insulin resistance syndrome).

65. A method for the treatment or prevention of diabetes, diabetes of Type 1, diabetes of Type 2, impaired glucose tolerance, hyperglycemia, and metabolic syndrome (metabolic syndrome X, insulin resistance syndrome), which method comprises administration to a subject in need thereof a therapeutically effective amount of a compound according to any one of embodiments 1-55 or the composition according to embodiment 61.

### EXAMPLES

Materials and Methods

List of Abbreviations
AIBN 2,2′-Azobisisobutyronitrile
AKT alias PKB, Protein kinase B
ALP achromobactor lyticus protease
Ar aryl
ARS alizarin red sodium
C18 octadecanyl (HPLC column)
CV column volume
DAST Diethylaminosulfur trifluoride
DBU 1,8-diazabicyclo(5.4.0)undec-7-en
DCM dichloromethane
DIC N,N-diisopropylcarbodiimide
DMF N,N-dimethylformamide
DIPEA N,N-diisopropylethylamine
EDC.HCl N-(3-dimethylaminopropyl)-N′-ethylcarbodiimide hydrochloride
EtOAc ethyl acetate
FFC Free fat cells (r, rats)
Fmoc-OSu 9-fluorenylmethyl N-succinimidyl carbonate
HATU 1-((dimethylamino)(dimethyliminio)methyl)-1H-[1,2,3]triazolo[4,5-b]pyridine 3-oxide hexafluorophosphate
HBTU 2-(1H-benzotriazol-1-yl)-1,1,3,3-tetramethyluronium hexafluorophosphate
HEPES 4-(2-hydroxyethyl)-1-piperazineethanesulfonic acid
HIR human insulin receptor (A=A isoform, B=B isoform)
HOBt 1-hydroxybenzotriazole
HONSU N-hydroxysuccinmide
HRMS high resolution mass spectrometry
HSA Human Serum Albumin
Kd displacement constant
LCMS liquid chromatography mass spectrometry
MeCN acetonitrile
mM millimolar
NBS N-bromosuccinimide
N.D. Non detectable
NMM N-methyl-morpholine
NMR nuclear magnetic resonance
NMP N-methyl-pyrrolidone
OEG 2-(2-aminoethoxy)ethoxy-acetic acid (oligoethyleneglycol amino acid)
OXYMA ethyl cyanohydroxyiminoacetate
RP-HPLC reverse-phase high performance liquid chromatography
Ph phenyl
SPA scitillation proximity assay
TFA trifluoroacetic acid
THF tetrahydrofuran
THPTA tris(3-hydroxypropyltriazolylmethyl)amine
TSTU succinimidyl-tetramethyluronium tetrafluoroborate
UPLC ultra performance liquid chromatography
WGA Wheat germ agglutinin

Preparation of Insulin Variants

### Example 1: Expression of Insulin Variants in Yeast and Transformation with ALP Etc

The insulin analogues were expressed in yeast using well-known techniques e.g. as disclosed in WO2017/032798. More specifically, the insulin analogues were expressed as single-chain precursors, which were isolated by ion-exchange capture, and cleaved to the 2-chain insulin analogues by treatment with ALP as described below.

Capture of the Precursor on SP Sepharose BB:

The yeast supernatant was loaded with a flow of 10-20 CV/h onto a column packed with SP Sepharose BB. A wash with 0.1 M citric acid pH 3.5 and a wash with 40% EtOH were performed. The analogue was eluted with 0.2 M sodium acetate pH 5.5/35% EtOH.

ALP Digestion:

The solution of single-chain precursor was adjusted to pH 9 and ALP enzyme was added 1:100 (w/w). The reaction was followed on UPLC. ALP cleavage pool was adjusted to pH 2.5 and diluted 2-fold in order to be prepared for RP-HPLC purification.

RP-HPLC Purification:

Purification was performed by RP-HPLC C18 as below:

Column: 15 um C18 50×250 mm 200 Å

Buffers:

A: 0.2% formic acid, 5% EtOH,

B: 0.2% formic acid, 50% EtOH

The gradient: 20-55% B-buffer.

Gradient: 20 CV

Flow 20 CV/h

Load g ˜5 g/l resin

Fractions were analysed by UPLC, pooled and freeze dried.

Insulin analogues prepared and used in the examples below:

desB30 human insulin (SEQ ID NO:1 and SEQ ID NO:11);

A14E B1K B2P B25H desB27 desB30 human insulin (SEQ ID NO:4 and SEQ ID NO:13);

A14E A22K B25H desB27 desB30 human insulin (SEQ ID NO:5 and SEQ ID NO:14);

A14E A22K B25H B27P B28G desB30 human insulin (SEQ ID NO:5 and SEQ ID NO:15);

A14E desB1-B2 B4K B5P desB30 human insulin (SEQ ID NO:4 and SEQ ID NO:16);

A14E desB1-B2 B3G B4K B5P desB30 human insulin (SEQ ID NO:4 and SEQ ID NO:17);

A14E B-1G B1K B2P desB30 human insulin (SEQ ID NO:4 and SEQ ID NO:18);

A22K desB30 human insulin (SEQ ID NO:6 and SEQ ID NO:11);

A22K B29R desB30 human insulin (SEQ ID NO:6 and SEQ ID NO:19);

A22K B22K B29R desB30 human insulin (SEQ ID NO:6 and SEQ ID NO:20);

A-2K A-1P desB30 human insulin (SEQ ID NO:7 and SEQ ID NO:11);

A21Q (GES)3K desB30 human insulin (SEQ ID NO:8 and SEQ ID NO:11);

A21Q (GES)6K desB30 human insulin (SEQ ID NO:9 and SEQ ID NO:11);

A21Q (GES)12K desB30 human insulin (SEQ ID NO:10 and SEQ ID NO:11);

B1-KPGGGGSGGGGSGGGGS desB30 human insulin (SEQ ID NO:1 and SEQ ID NO:21);

B1-KPGGGGSGGGGSGGGGS A14E B25H desB30 human insulin (SEQ ID NO:4 and SEQ ID NO:22);

B1-GKPGGGGSGGGGSGGGGS desB30 human insulin (SEQ ID NO:1 and SEQ ID NO:23);

B1-GKPGGGGSGGGGS desB30 human insulin (SEQ ID NO:1 and SEQ ID NO:24);

B1-GKPGGGGS desB30 human insulin (SEQ ID NO:1 and SEQ ID NO:25);

B1-GKPG desB30 human insulin (SEQ ID NO:1 and SEQ ID NO:26);

B1-GKPRGFFYTPGGGGSGGGGS desB30 human insulin (SEQ ID NO:1 and SEQ ID NO:27); and

B1-TYFFGRKPDGGGGSGGGGSGGGGS desB30 human insulin (SEQ ID NO:1 and SEQ ID NO:28).

B1-GKPRGFFYTPGGGGSGGGGS desB30 human insulin means desB30 human insulin extended from B1 with GKPRGFFYTPGGGGSGGGGS (written from new N-terminal G with C-terminal S connected to B1 of desB30 human insulin). A21Q (GES)3K desB30 human insulin means insulin extended from A21Q with GESGESGESK (written from new N-terminal G connected to C-terminal A21Q). Similar for the other B1 and A21 extended insulin analogues. B-1 means the position N-terminally from B1, e.g. B-1G means N-terminal extension of insulin B1 with G.

Preparation of Building Blocks

The intermediates and final products are given numbers within each example to make reading easier. The same numbers are used across the examples, but the numbers are unambiguous within each example.

### Example 2: O-succinimidyl 3,5-bis[[[3-fluoro-4-(4,4,5,5-tetramethyl-1,2-dioxaborolan-2-yl)benzoyl]amino]methy]benzoate

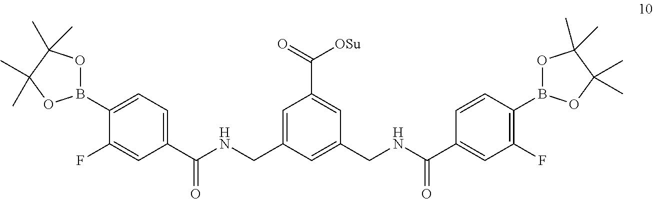

Mixture of 2-fluoro-4-carboxyphenylboronic acid (1, 8.44 g, 45.9 mmol), pinacol (5.42 g, 45.9 mmol) and magnesium sulfate (60 g) in tetrahydrofuran (110 mL) was stirred overnight at room temperature. The suspension was filtered through celite pad, the filtrate was evaporated and dried in vacuo to yield 3-fluoro-4-(4,4,5,5-tetramethyl-1,32-dioxaborolan-2-yl)benzoic acid (2) as beige powder. Yield: 10.4 g (85%). ¹H NMR spectrum (300 MHz, CDCl₃, δH): 7.93-7.80 (m, 2H); 7.75 (d, J=9.4 Hz, 1H); 1.39 (s, 12H).

The carboxylic acid 2 (10.3 g, 38.6 mmol) was dissolved in dichloromethane (130 mL). 1-5 Hydroxy-pyrrolidine-2,5-dione (HOSu, 8.89 g, 77.2 mmol) and N-(3-dimethylaminopropyl)-N′-ethylcarbodiimide hydrochloride (EDC.HCl, 14.8 g, 77.2 mmol) were added. Resulting mixture was stirred overnight at room temperature. The reaction mixture was partitioned between ethyl acetate (130 mL) and 0.5 M aqueous solution of hydrochloric acid (130 mL). Organic layer was washed with 0.5 M aqueous solution of hydrochloric acid (3×120 mL), dried over anhydrous sodium sulfate, filtered and evaporated. The residue was dissolved in dichloromethane (40 mL) and precipitated by addition of cyclohexane (130 mL). The product was collected by filtration, washed with cyclohexane and dried in vacuo to yield succinimidyl 3-fluoro-4-(4,4,5,5-tetramethyl-1,3,2-dioxaborolan-2-yl)benzoate (3) as white powder. Yield: 13.9 g (99%). ¹H NMR spectrum (300 MHz, CDCl₃, δH): 7.93-7.84 (m, 2H); 7.77 (d, J=9.4 Hz, 1H); 2.92 (s, 4H); 1.39 (s, 12H).

3,5-Dimethylbenzoic acid 4 827.6 g, 18.4 mol) was suspended in methanol (80 mL) and treated with concentrated sulfuric acid (8 mL). The mixture was refluxed for 2 days. After neutralization with sodium carbonate (50 g), the mixture was dissolved in water (250 mL) and extracted with diethyl ether (2×300 mL). The organic phases were dried over anhydrous sodium sulfate, filtered and evaporated to dryness affording methyl 3,5-dimethylbenzoate (5) as pale yellow oil. Yield: 29.3 g (97%). ¹H NMR spectrum (300 MHz, CDCl₃, δH): 7.67 (s, 2H); 7.19 (s, 1H); 3.91 (s, 3H); 2.37 (s, 6H).

A mixture of the above methyl 3,5-dimethylbenzoate 5 (29.3 g, 178 mmol), N-bromosuccinimide (NBS, 111 g, 623 mmol) and a spatula of azobisisobutyronitrile in methyl formate (450 mL) was irradiated with visible light while heating to reflux for 20 hours. The solvent was evaporated and the residue was dissolved in dichloromethane (200 mL). The precipitated succinimide was filtered off and the filtrate was washed with saturated aqueous solution of sodium sulfite (2×150 mL). The organic layer was dried over anhydrous sodium sulfate, filtered and evaporated. The residue was purified by flash column chromatography (Silicagel 60, 0.040-0.063 mm; eluent:hexane/ethyl acetate 15:1). The product was crystallized from ethyl acetate/cyclohexane mixture giving methyl 3,5-bis(bromomethyl)benzoate (6) as white solid. Yield: 25.6 g (45%). RF (SiO₂, hexanes/ethyl acetate 9:1): 0.50. ¹H NMR spectrum (300 MHz, CDCl₃, δH): 8.02-7.95 (m, 2H); 7.62 (s, 1H); 4.51 (s, 4H); 3.94 (s, 3H).

A suspension of the above bromide 6 (25.3 g, 78.6 mmol) and sodium diformylamide (20.9 g, 220 mol) in dry acetonitrile (350 mL) was refluxed for 4 hours. After removal of a white solid by filtration, the solvent was evaporated. Recrystallization from ethyl acetate/cyclohexane mixture afforded methyl 3,5-bis((N-formylformamido)methyl)benzoate (7) as white powder.

Yield: 21.0 g (88%). ¹H NMR spectrum (300 MHz, DMSO-d6, δH): 9.08 (s, 4H); 7.72 (s, 2H); 7.44 (s, 1H); 4.70 (s, 4H); 3.84 (s, 3H).

Benzoate 7 (20.9 g, 68.5 mmol) was dissolved in a mixture of 1,4-dioxane (220 mL) and concentrated hydrochloric acid (280 mL) and heated for 2 hours to reflux. After cooling down to room temperature, a flow of air was passed through the solution. Product began to precipitate. After 1 hour, the solvent was evaporated and product was recrystallized from methanol/diethyl ether mixture affording 3,5-bis(aminomethyl)benzoic acid dihydrochloride (8) as white powder. Yield: 17.1 g (98%). ¹H NMR spectrum (300 MHz, DMSO-d6, 6H): 13.26 (bs, 1H); 8.65 (bs, 6H); 8.10 (s, 2H); 7.88 (s, 1H); 4.08 (s, 4H).

Dihydrochloride 8 (2.08 g, 8.20 mmol) was dissolved in water (20 mL). Subsequently N,N-diisopropylethylamine (5.73 mL, 32.9 mmol), N,N-dimethylformamide (40 mL) and activated ester (3, 5.97 g, 16.4 mmol) were added. The mixture was stirred overnight at room temperature; then it was acidified by 1 M aqueous solution of hydrochloric acid. The solvent was co-evaporated with toluene three times. The residue was dissolved in dichloromethane/toluene mixture (1:1, 100 mL) and treated with pinacol (1.40 g, 11.8 mmol). The mixture was evaporated three times from toluene. The residue was dissolved in ethyl acetate (250 mL) and washed with water (3×150 mL). Organic layer was dried over anhydrous sodium sulfate, filtered and evaporated. The residue was dissolved in dichloromethane (10 mL) and product started to precipitate. Cyclohexane was added (170 mL). The precipitate was collected by filtration, washed with cyclohexane and dried in vacuo to give 3,5-bis((3-fluoro-4-(4,4,5,5-tetramethyl-1,3,2-dioxaborolan-2-yl)benzamido)methyl)benzoic acid (9) as white powder. Yield: 4.18 g (75%). ¹H NMR spectrum (300 MHz, DMSO-d6, δH): 12.96 (bs, 1H); 9.27 (t, J=5.9 Hz, 2H); 7.82-7.67 (m, 6H); 7.64-7.56 (m, 2H); 7.53 (s, 1H); 4.58-4.44 (m, 4H); 1.31 (s, 24H).

The above acid 9 (4.17 g, 6.20 mmol) was dissolved in acetonitrile/N,N-dimethylformamide mixture (4:1, 100 mL). N-hydroxysuccinimide (HOSu, 0.85 g, 7.40 mmol) was added. The mixture was cooled down to 0° C. followed by addition of N,N-dicyclohexylcarbodiimide (DCC, 1.53 g, 7.40 mmol). The mixture was stirred for 30 minutes at 0° C. and overnight at room 5 temperature. The insoluble by-product was filtered off and the filtrate was evaporated. The residue was dissolved in ethyl acetate (250 mL) and washed with water (2×150 mL). Organic layer was dried over anhydrous sodium sulfate, filtered and evaporated. The residue was dissolved in dichloromethane (10 mL) and product started to precipitate. Cyclohexane was added (170 mL). The precipitate was collected by filtration, washed with cyclohexane and dried in vacuo to give O-succinimidyl 3,5-bis[[[3-fluoro-4-(4,4,5,5-tetramethyl-1,3,2-dioxaborolan-2-yl)benzoyl]amino]methyl]benzoate (10) as white powder. Yield: 4.62 g (97%).

¹H NMR spectrum (300 MHz, DMSO-d6, δH): 9.31 (t, J=5.7 Hz, 2H); 7.93 (s, 2H); 7.79-7.68 (m, 5H); 7.63-7.56 (m, 2H); 4.60-4.50 (m, 4H); 2.87 (s, 4H); 1.31 (s, 24H). LC-MS: 773.4 (M+H)⁺, calculated 773.4.

### Example 3: O-succinimidyl N,N-bis(3-fluoro-4-(4,4,5,5-tetramethyl-1,3,2-dioxaborolan-2-yl)-benzamido-Lys-beta Ala

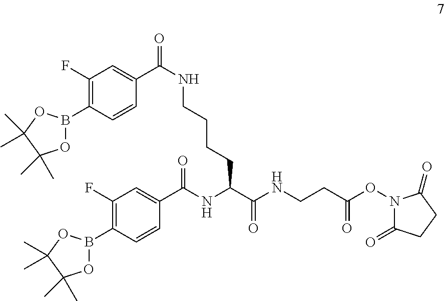 [SMILES: CC1(C)OB(c2ccc(C(=O)NCCCC[C@H](NC(=O)c3ccc(B4OC(C)(C)C(C)(C)O4)c(F)c3)C(=O)NCCC(=O)ON3C(=O)CCC3=O)cc2F)OC1(C)C]

2-Chlorotrityl resin 100-200 mesh 1.8 mmol/g 1 (53.3 g, 96.0 mmol) was left to swell in dry dichloromethane (350 mL) for 20 minutes. Then the resin was filtered and washed with dry dichloromethane (300 mL). After that the solution of Fmoc-Ala-OH (24.9 g, 80.0 mmol) and N,N-diisopropylethylamine (55.7 mL, 320 mmol) in dry dichloromethane (250 mL) was added to the resin and the mixture was shaken overnight. After that the resin was filtered and treated with a solution of N,N-diisopropylethylamine (50 mL) in methanol/dichloromethane mixture (4:1, 2×5 min, 2×250 mL). Then the resin was filtered and washed with N,N-dimethylformamide (2×250 mL), dichloromethane (2×250 mL) and N,N-dimethylformamide (2×250 mL). Fmoc group was removed by treatment with 20% solution of piperidine in N,N-dimethylformamide (1×5 min, 1×30 min, 2×250 mL). After that the resin was filtered and 5 washed with N,N-dimethylformamide (2×250 mL), dichloromethane (2×250 mL) and N,N-dimethylformamide (2×250 mL). After that the solution Fmoc-L-Lys(Boc)-OH (56.2 g, 120 mmol), 5-chloro-1-((dimethylamino)(dimethyliminio)methyl)-1H-benzo[d][1,2,3]triazole-3-oxide tetrafluoroborate (TCTU, 42.7 g, 120 mmol) and N,N-diisopropylethylamine (34.8 mL, 200 mmol) in N,N-dimethylformamide (180 mL) was added to resin and mixture was shaken for 3 hours. After that the resin was filtered and washed with N,N-dimethylformamide (2×250 mL), dichloromethane (2×250 mL) and N,N-dimethylformamide (2×250 mL). Fmoc group was removed by treatment with 20% solution of piperidine in N,N-dimethylformamide (1×5 min, 1×30 min, 2×300 mL). Then the resin was filtered and washed with N,N-dimethylformamide (2×300 mL), dichloromethane (2×300 mL), methanol (2×300 mL) and dichloromethane (10×300 mL). The product was cleaved from the resin by the treatment with 2,2,2-trifluoroethanol (300 mL) overnight. Resin was filtered off and washed with dichloromethane (2×200 mL), 2-propanol (2×200 mL) and dichloromethane (2×200 mL).

The solvent was removed under reduced pressure and the residue was triturated in diethyl ether (2×300 mL). After filtration and drying we obtained L-Lys(Boc)-beta-Ala (2) as off-white powder. Yield: 13.3 g (56%). ¹H NMR spectrum (300 MHz, AcOD-d₄, δ_{H}): 4.20 (t, J=7.1 Hz, 1H); 3.66-3.46 (m, 2H); 3.17-3.00 (m, 2H); 2.65 (t, J=6.4 Hz, 2H); 1.97-1.80 (m, 2H); 1.60-1.30 (m, 13H).

95% aqueous solution of trifluoroacetic acid (60 mL) was added to the suspension of 2 (13.2 g, 41.6 mmol) in dichloromethane (50 mL) and the whole mixture was stirred for 2 hours.

Then the solvent was removed under reduced pressure and the residue was dried in vacuo to give L-Lys-beta-Ala TEA salt (3) as brown oil. Yield: 18.5 g (100%). ¹H NMR spectrum (300 MHz, AcOD-d₄, δ_{H}): 4.24 (t, J=6.7 Hz, 1H); 3.71-3.46 (m, 2H); 3.09 (t, J=7.5 Hz, 2H); 2.66 (t, J=6.6 Hz, 2H); 2.01-1.89 (m, 2H); 1.85-1.68 (m, 2H); 1.60-1.46 (m, 2H).

Triethylamine (14.1 mL, 101 mmol) was added to the solution of 3 (15.0 g, 33.7 mmol) in acetonitrile to give an off-white precipitate. After filtration and drying was obtained L-Lys-beta-Ala (4) as white hygroscopic powder. Yield: 7.30 g (100%). ¹H NMR spectrum (300 MHz, AcOD-d₄, δ_{H}): 4.21 (t, J=6.4 Hz, 1H); 3.72-3.45 (m, 2H); 3.08 (t, J=7.4 Hz, 2H); 2.65 (t, J=6.0 Hz, 2H); 2.00-1.88 (m, 2H); 1.83-1.66 (m, 2H); 1.59-1.43 (m, 2H).

Succinimidyl 3-fluoro-4-(4,4,5,5-tetramethyl-1,3,2-dioxaborolan-2-yl)benzoate 5 (5.00 g, 13.8 mmol) was added to the suspension of 4 (3.00 g, 13.8 mmol) and triethylamine (7.74 mL, 55.5 mmol) in dry acetonitrile (80 mL) and the whole mixture was stirred overnight. Then the solvent was removed under reduced pressure and co-evaporated with toluene three times. After that the ethyl acetate (70 mL) was added and the mixture was washed with water (3×50 mL). Organic layer was separated, dried over anhydrous sodium sulfate, filtered and evaporated. The residue was dissolved in dichloromethane (2 mL) and added dropwise to vigorously stirred cyclohexane (100 mL). The precipitate was collected by filtration, washed 10 with cyclohexane and dried in vacuo to give N,N-bis(3-fluoro-4-(4,4,5,5-tetramethyl-1,3,2-dioxaborolan-2-yl)benzamido-Lys-beta-Ala (6) as white powder. Yield: 2.31 g (47%). ¹H NMR spectrum (300 MHz, AcOD-d₄, δ_{H}): 7.83-7.73 (m, 2H); 7.71-7.45 (m, 4H); 4.77 (t, J=7.2 Hz, 1H); 3.61-3.39 (m, 4H); 2.64 (t, J=6.2 Hz, 2H); 2.00-1.80 (m, 2H); 1.77-1.47 (m, 4H); 1.37 (s, 24H).

N-hydroxysuccinimide (HOSu, 0.97 g, 8.41 mmol) was added to the solution of 6 (2.00 g, 2.80 mmol) in dry acetonitrile (70 mL). The mixture was cooled down to 0° C. followed by addition of N,N-dicyclohexylcarbodiimide (DCC, 0.87 g, 4.20 mmol). After 30 minutes the reaction mixture was allowed to warm to room temperature and stirred overnight. The insoluble by-product was filtered off and the filtrate was evaporated. The residue was dissolved in ethyl acetate (100 mL) and washed with 1 M aqueous solution of hydrochloric acid (3×70 mL), water (70 mL) and brine (70 mL). Organic layer was separated, dried over anhydrous sodium sulfate, filtered and evaporated. The residue was co-evaporated with pinacol in toluene five times. Then the residue was dissolved in ethyl acetate (100 mL) and 25 washed with 0.1 M aqueous solution of hydrochloric acid (70 mL), water (70 mL) and brine (70 mL). Organic layer was separated, dried over anhydrous sodium sulfate, filtered and evaporated. The residue was dissolved in dichloromethane (3 mL) and added dropwise to vigorously stirred mixture of cyclohexane/diethyl ether (10:1, 110 mL). The precipitate was collected by filtration, washed with cyclohexane and dried in vacuo. The residue cyclohexane was removed by co-evaporation with dichloromethane five times. After drying O-succinimidyl N,N-bis(3-fluoro-4-(4,4,5,5-tetramethyl-1,3,2-dioxaborolan-2-yl)benzamido-Lys-beta-Ala (7) was obtained as off-white foam. Yield: 1.12 g (48%). ¹H NMR spectrum (300 MHz, CDCl₃, 6H): 7.83-7.71 (m, 2H); 7.62-7.40 (m, 4H); 7.19 (d, J=7.7 Hz, 1H); 7.02 (t, J=6.1 Hz, 1H); 6.68 (t, J=5.6 Hz, 1H); 4.67 (m, 1H); 3.73-3.62 (m, 2H); 3.44 (q, J=6.2 Hz, 2H); 2.90-2.78 (m, 6H); 2.07-1.60 (m, 4H); 1.53-1.30 (m, 26H). LC-MS: 810.5 (M+H)⁺, calculated 810.4.

### Example 4: O-succinimidyl N,N-bis(3-fluoro-4-(4,4,5,5-tetramethyl-1,32-dioxaborolan-2-yl)benzamido-Lys-Gly

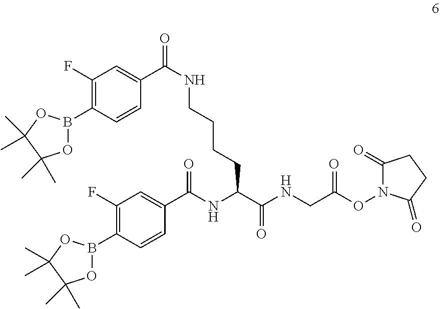 [SMILES: CC1(C)OB(c2ccc(C(=O)NCCCC[C@H](NC(=O)c3ccc(B4OC(C)(C)C(C)(C)O4)c(F)c3)C(=O)NCC(=O)ON3C(=O)CCC3=O)cc2F)OC1(C)C]

Mixture of 2-fluoro-4-carboxyphenylboronic acid 1 (4.95 g, 27.0 mmol), pinacol (3.21 g, 27.2 mmol) and magnesium sulfate (450 g) in tetrahydrofuran (90 mL) was stirred overnight at room temperature. The suspension was filtered through celite pad, the filtrate was evaporated and dried in vacuo to yield 3-fluoro-4-(4,4,5,5-tetramethyl-1,3,2-dioxaborolan-2-yl)benzoic acid (2) as yellow powder. Yield: 7.06 g (98%). ¹H NMR spectrum (300 MHz, CDCl₃, δ_{H}): 7.93-7.80 (m, 2H); 7.76 (d, J=9.4 Hz, 1H); 1.39 (s, 12H).

The carboxylic acid 2 (7.05 g, 26.5 mmol) was dissolved in dichloromethane (100 mL). N-Hydroxysuccinimide (HOSu, 6.10 g, 53.0 mmol) and N-(3-dimethylaminopropyl)-N′-ethylcarbodiimide hydrochloride (EDC.HCl, 10.2 g, 53.0 mol) were added. Resulting mixture was stirred overnight at room temperature. The reaction mixture was partitioned between ethyl acetate (110 mL) and 0.1 M aqueous solution of hydrochloric acid (110 mL). Organic layer was washed with 0.1 M aqueous solution of hydrochloric acid (2×100 mL) and brine (1×100 mL), dried over anhydrous sodium sulfate, filtered and evaporated. The residue was dissolved in dichloromethane (20 mL) and precipitated by addition of cyclohexane (120 mL). The product was collected by filtration, washed with cyclohexane and dried in vacuo to yield succinimidyl 3-fluoro-4-(4,4,5,5-tetramethyl-1,3,2-dioxaborolan-2-yl)benzoate (3) as white powder. Yield: 9.60 g (99%). ¹H NMR spectrum (300 MHz, DMSO-d₆, δ_{H}): 7.96-7.89 (m, 2H); 7.79 (d, J=9.4 Hz, 1H); 2.91 (s, 4H); 1.33 (s, 12H).

L-Lys-Gly TFA salt 4 (2.67 g, 6.20 mmol) was dissolved in water (20 mL). Subsequently N,N-diisopropylethylamine (4.32 mL, 24.8 mmol), N,N-dimethylformamide (40 mL) and 2,5-dioxopyrrolidin-1-yl 3-fluoro-4-(4,4,5,5-tetramethyl-1,3,2-dioxaborolan-2-yl)benzoate (3, 4.50 g, 12.4 mmol) were added. The mixture was stirred overnight at room temperature; then it 5 was acidified by 1 M aqueous solution of hydrochloric acid. The solvent was co-evaporated with toluene three times. The residue was dissolved in dichloromethane/toluene mixture (1:1, 100 mL) and treated with pinacol (1.00 g, 8.46 mmol). The mixture was evaporated three times from toluene. The residue was dissolved in ethyl acetate (250 mL) and washed with water (3×150 mL). Organic layer was dried over anhydrous sodium sulfate, filtered and evaporated. The residue was dissolved in dichloromethane (10 mL) and added dropwise to a cold cyclohexane (200 mL). The precipitate was collected by filtration, washed with cyclohexane and dried in vacuo. The solid was dissolved in dichloromethane (10 mL). Diethyl ether (10 mL) and cyclohexane (150 mL) were added. The solvent was decanted, the residue was dried in vacuo to yield N,N′-bis(3-fluoro-4-(4,4,5,5-tetramethyl-1,3,2-dioxaborolan-2-yl)benzoyl)-L-lysylglycine (5) as beige solid. Yield: 1.60 g (37%). ¹H NMR spectrum (300 MHz, CDCl₃, δ_{H}): 7.91-7.79 (m, 2H); 7.78-7.64 (in, 2H); 7.58-7.34 (m, 4H); 7.19-7.07 (m, 1H); 4.86-4.72 (m, 1H); 4.15-3.88 (m, 2H); 3.47-3.25 (m, 2H); 2.00-1.74 (m, 2H); 1.66-1.52 (m, 2H); 1.50-1.37 (in, 2H); 1.34 (s, 24H). LC-MS: 699.3 (M+H)⁺, 617.2 (M+H−pinacol)⁺, 535.0 (M+H−2×pinacol)⁺.

The above acid 5 (1.59 g, 2.30 mmol) was dissolved in dichloromethane (70 mL). N-Hydroxysuccinimide (HOSu, 0.31 g, 2.70 mmol) and N-(3-dimethylaminopropyl)-N′-ethylcarbodiimide hydrochloride (EDC.HCl, 0.65 g, 3.40 mmol) were added. The reaction mixture was stirred for 5 hours at room temperature. The mixture was washed with 0.1 M aqueous solution of hydrochloric acid (2×80 mL) and brine (1×80 mL). Organic layer was dried over anhydrous sodium sulfate, filtered and evaporated to dryness giving 0-succinimidyl N,N-bis(3-fluoro-4-(4,4,5,5-tetramethyl-1,3,2-dioxaborolan-2-yl)benzamido-Lys-Gly (6) as beige solid. Yield: 1.29 g (70%). ¹H NMR spectrum (300 MHz, DMSO-d6, δ_{H}): 8.73-8.53 (m, 3H); 7.78-7.61 (m, 5H); 7.54 (d, J=10.4 Hz, 1H); 4.52-4.40 (m, 1H); 4.36-4.17 (m, 2H); 3.29-3.17 (m, 2H); 2.81 (s, 4H); 1.87-1.68 (m, 2H); 1.59-1.47 (m, 2H); 1.46-1.36 (m, 2H); 1.31 (s, 24H). LC-MS: 796.4 (M+H)⁺, calculated 796.4.

### Example 5: 2-((23-(3,5-Bis((3-(3-acetoxy-2,2-bis(acetoxymethyl)propoxy)propanamido)methylbenzamido)-7,16-dioxo-3,9,12,18,21-pentaoxa-6,15-diazatricosyl)oxy)-N-(4-formylbenzyl)acetamide

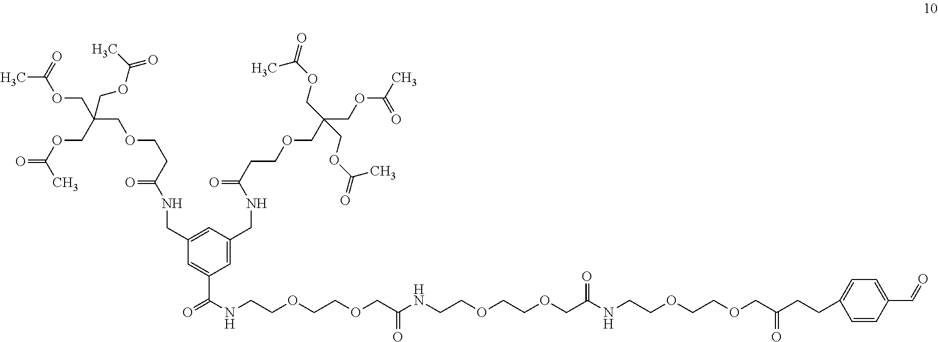 [SMILES: CC(=O)OCC(COCCC(=O)NCc1cc(CNC(=O)CCOCC(COC(C)=O)(COC(C)=O)COC(C)=O)cc(C(=O)NCCOCCOCC(=O)NCCOCCOCC(=O)NCCOCCOCC(=O)CCc2ccc(C=O)cc2)c1)(COC(C)=O)COC(C)=O]

A mixture of pentaerythritol (136 g, 1.00 mol), sodium hydroxide (8.00 g, 200 mmol), dimethyl sulfoxide (200 mL) and water (18 mL) was heated at 80° C. until a clear solution was formed (overnight). tert-Butyl acrylate (2, 174 mL, 1.20 mol) was added and the resulting mixture was heated at 80° C. for 24 hours; then it was cooled to room temperature, diluted with water 10 (200 mL) and extracted with ethyl acetate (3×400 mL). Combined organic layers were washed with water (400 mL) and brine (100 mL). Since the aqueous washes contained the product (3), they were combined and re-extracted with ethyl acetate (2×200 mL). All ethyl acetate fractions were combined, dried over anhydrous sodium sulfate and evaporated to dryness. The residue was purified by flash column chromatography (Silicagel 60, 0.040-0.063 mm; eluent:dichloromethane/methanol 99:1-90:10) to give tert-butyl 3-(3-hydroxy-2,2-bis(hydroxymethyl)propoxy)propanoate (3) as colorless oil.

Yield: 39.7 g (15%). RF (SiO2, dichloromethane/methanol 9:1): 0.30.

1H NMR spectrum (300 MHz, CDCl₃, δH): 3.67 (t, J=5.6 Hz, 2H); 3.65 (s, 6H); 3.52 (s, 2H); 2.73 (bs, 3H); 2.49 (t, J=5.7 Hz, 2H); 1.46 (s, 9H). LC-MS: 287.2 (M+Na)+.

Acetic anhydride (95.6 mL, 350 mmol) was added to a solution of the above tert-butyl 3-(3-hydroxy-2,2-bis(hydroxymethyl)propoxy)propanoate (3, 74.5 g, 281 mmol) and N,N-diisopropylethylamine (88.1 mL, 506 mmol) in dry dichloromethane (600 mL) at 0° C. The cooling bath was removed and the resulting solution was stirred at room temperature 25 overnight. The volatiles were removed in vacuo; the residue was re-dissolved in ethyl acetate (2 L) and washed with water (600 mL), 0.5 M aqueous hydrochloric acid (1.2 L), water (600 mL), 10% aqueous solution of potassium hydrogencarbonate (600 mL), water (600 mL) and brine (230 mL). The organic layer was dried over anhydrous sodium sulfate and evaporated to dryness. The residue was purified by flash column chromatography (Silicagel 60, 0.040-0.063 mm; eluent:cyclohexane/ethyl acetate 9:1-8:2) to afford 2-(acetoxymethyl)-2-((3-(tert-butoxy)-3-oxopropoxy)methyl)propane-1,3-diyl diacetate (4) as colorless oil.

Yield: 86.7 g (79%). RF (SiO2, hexanes/ethyl acetate 3:2): 0.40. ¹H NMR spectrum (300 MHz, CDCl₃, δH): 4.11 (s, 6H); 3.65 (t, J=6.2 Hz, 2H); 3.44 (s, 2H); 2.45 (t, J=6.3 Hz, 2H); 2.06 (s, 9H); 1.46 (s, 9H). LC-MS: 413.2 (M+Na)+.

Trifluoroacetic acid (300 mL) was added to a solution of the above 2-(acetoxymethyl)-2-((3-(tert-butoxy)-3-oxopropoxy)methyl)propane-1,3-diyl diacetate (4, 86.0 g, 220 mmol) in dichloromethane (100 mL). The resulting solution was stirred at room temperature for 2 hours, then it was evaporated to dryness and the residue evaporated from toluene (3×150 mL). The residue was purified by flash column chromatography (Silicagel 60, 0.040-0.063 mm; eluent:dichloromethane/methanol 10:0-9:1), fractions containing product were evaporated to give the title compound (5) as light brown oil.

Yield: 70.4 g (96%). RF (SiO2, hexanes/ethyl acetate 1:1): 0.25. ¹H NMR spectrum (300 MHz, CDCl₃, δH): 4.10 (s, 6H); 3.69 (t, J=6.1 Hz, 2H); 3.46 (s, 2H); 2.60 (t, J=6.1 Hz, 2H); 2.06 (s, 9H). LC-MS: 357.2 (M+Na)+.

2-Chlorotrityl resin 100-200 mesh 1.5 mmol/g (10.7 g, 16.0 mmol) was left to swell in dry dichloromethane (100 mL) for 20 minutes. A solution of {2-[2-(9H-fluoren-9-ylmethoxycarbonylamino)-ethoxy]-ethoxy}-acetic acid (Fmoc-OEG-OH, 4.12 g, 10.7 mmol) and N,N-diisopropylethylamine (7.07 mL, 40.6 mmol) in dry dichloromethane (20 mL) was added to resin and the mixture was shaken for 16 hours. Resin was filtered and treated with a solution of N,N-diisopropylethylamine (3.72 mL, 21.4 mmol) in methanol/dichloromethane mixture (2:8, 2×5 min, 2×50 mL). Then resin was washed with N,N-dimethylformamide (2×50 mL), dichloromethane (2×50 mL) and N,N-dimethylformamide (2×50 mL). Fmoc group was removed by treatment with 20% piperidine in N,N-dimethylformamide (1×5 min, 1×10 min, 1×30 min, 3×50 mL). Resin was washed with N,N-dimethylformamide (2×50 mL), 2-propanol (2×50 mL) and dichloromethane (2×50 mL). Solution of {2-[2-(9H-fluoren-9-ylmethoxycarbonylamino)-ethoxy]-ethoxy}-acetic acid (Fmoc-OEG-OH, 6.17 g, 16.0 mmol), 5-chloro-1-((dimethylamino)(dimethyliminio)methyl)-1H-benzo[d][1,2,3]triazole 3-oxide tetrafluoroborate (TCTU, 5.70, 16.0 mmol) and N,N-diisopropylethylamine (5.02 mL, 28.8 mmol) in N,N-dimethylformamide (50 mL) was added to resin and mixture was shaken for 1 hour. Then resin was washed with N,N-dimethylformamide (2×50 mL), dichloromethane (2×50 mL) and N,N-dimethylformamide (2×50 mL). Fmoc group was removed by treatment with 20% piperidine in N,N-dimethylformamide (1×5 min, 1×10 min, 1×30 min, 3×50 mL). Resin was washed with N,N-dimethylformamide (2×50 mL), 2-propanol (2×50 mL), dichloromethane (2×50 mL). Solution of {2-[2-(9H-fluoren-9-ylmethoxycarbonylamino)-ethoxy]-ethoxy}-acetic acid (Fmoc-OEG-OH, 6.17 g, 16.0 mmol), 5-chloro-1-((dimethylamino)(dimethyliminio)methyl)-1H-benzo[d][1,2,3]triazole 3-oxide tetrafluoroborate (TCTU, 5.70, 16.0 mmol) and N,N-diisopropylethylamine (5.02 mL, 28.8 mmol) in N,N-dimethylformamide (50 mL) was added to resin and mixture was shaken for 1 hour. Then resin was washed with N,N-dimethylformamide (2×50 mL), dichloromethane (2×50 mL) and N,N-dimethylformamide (2×50 mL). Fmoc group was removed by treatment with 20% piperidine in N,N-dimethylformamide (1×5 min, 1×10 min, 1×30 min, 3×50 mL). Resin was washed with N,N-dimethylformamide (2×50 mL), 2-propanol (2×50 mL), dichloromethane (2×50 mL). Solution of 3,5-bis(((((9H-fluoren-9-yl)methoxy) carbonyl)amino)methyl)benzoic acid (10.0 g, 16.0 mmol), 5-chloro-1-((dimethylamino)(dimethyliminio)methyl)-1H-benzo[d][1,2,3]triazole 3-oxide tetrafluoroborate (TCTU, 5.70, 16.0 mmol) and N,N-diisopropylethylamine (5.02 mL, 28.8 mmol) in N,N-dimethylformamide (50 mL) was added to resin and mixture was shaken for 1 hour. Then resin was washed with N,N-dimethylformamide (2×50 mL), dichloromethane (2×50 mL) and N,N-dimethylformamide (2×50 mL). Fmoc group was removed by treatment with 20% piperidine in N,N-dimethylformamide (1×5 min, 1×10 min, 1×30 min, 3×50 mL). Resin was washed with N,N-dimethylformamide (2×50 mL), 2-propanol (2×50 mL), dichloromethane (2×50 mL). Solution of 3-(3-acetoxy-2,2-bis(acetoxymethyl)propoxy)propanoic acid (5, 10.7 g, 32.0 mmol), ethyl cyano-glyoxylate-2-oxime (OXYMA, 4.55 g, 32.0 mmol), 2,4,6-collidine (7.68 mL, 6.99 mmol) and N,N-diisopropylcarbodiimide (DIC, 4.96 g, 32.0 mmol) in N,N-dimethylformamide (40 mL) was added to resin and mixture was shaken for 1 hour. Resin was filtered and washed with N,N-dimethylformamide (3×50 mL), dichloromethane (4×50 mL), methanol (4×50 mL) and dichloromethane (7×50 mL). The product was cleaved from the resin by the treatment with mixture trifluoroacetic acid/dichloromethane (1:1, 50 mL) overnight. Resin was filtered off and washed with dichloromethane (2×50 mL). The solvent was removed under reduced pressure. The residue was purified by column chromatography (silicagel 60, 0.040-0.063 mm; eluent:dichloromethane/methanol 100:0 to 90:10) giving compound (8) contaminated with nethylester and partially deacetylated products. Compound (8) was dissolved in dioxane and solution of lithium hydroxide (3.42 g, 81.5 mmol) in water (160 mL) was added. The mixture was stirred for 30 minutes, then neutralized with 1 M hydrochloric acid (80 mL) and freeze-dried. Deacetylated 8 was dissolved in mixture of dichloromethane (50 mL) and N,N-dimethylformamide (10 mL), then pyridine (50 mL) and acetic anhydride (30.5 mL) was added. The mixture was stirred for 72 hours and then evaporated multiple times from N,N-dimethylformamide to give desired compound 8 as brown oil.

Yield: 13.2 g (99%). LC-MS: 1249 (M+H)+.

The above compound (8, 15.6 g, 12.5 mmol), 2,4,6-collidine (14.9 mL, 113 mmol), [1,2,3]triazolo[4,5-b]pyridine-1-ol (HOAt, 5.10 g, 37.6 mmol) and N-(3-dimethylaminopropyl)-N-ethylcarbodiimide hydrochloride (EDC.HCl, 7.89 g, 41.3 mmol) were dissolved in dichloromethane (170 mL) and N,N-dimethylformamide (20 mL). 4-Formyl-benzyl-ammonium chloride (7.08 g, 41.3 mmol) was added. The mixture was stirred at room temperature for 48 hours and evaporated in vacuo. The residue was purified by HPLC (Deltapak, 018, 5 m, 50×500 mm, acetonitrile/water, 15:85 to 25:75, during 30 min, 25:75 to 50:50, during 170 min+0.05% TFA) to give title compound 10 as brownish oil. Yield: 1.96 g (12%). ¹H NMR spectrum (300 MHz, CDCl₃, δH): 9.98 (s, 1H); 7.84 (d, J=8.1 Hz, 2H); 7.56-7.41 (m, 3H); 7.39-7.33 (m, 1H); 7.25-7.14 (m, 2H); 7.09-7.00 (m, 1H); 4.56 (d, J=6.2 Hz, 2H); 4.46-4.40 (m, 4H); 4.09-3.96 (m, 16H); 3.91 (s, 2H); 3.73-3.56 (m, 20H); 3.52 (t, J=5.1 Hz, 4H); 3.45-3.32 (m, 8H); 2.49 (t, J=5.8 Hz, 4H); 2.05 (s, 18H). LC-MS: 1366 (M+H)+.

### Example 6: Boc-Lys(Boc)-OEG3-benzaldehyde

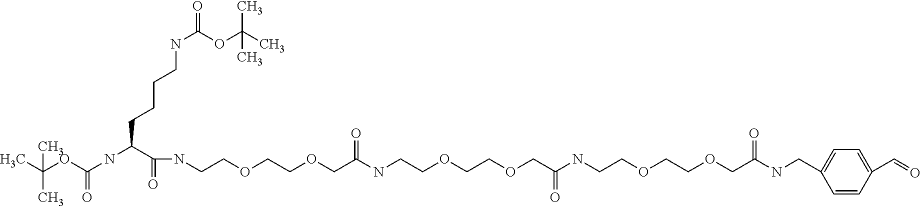 [SMILES: CC(C)(C)OC(=O)NCCCC[C@H](NC(=O)OC(C)(C)C)C(=O)NCCOCCOCC(=O)NCCOCCOCC(=O)NCCOCCOCC(=O)NCc1ccc(C=O)cc1]

The compound of example 6 was prepared similarly to compound of example 5 from Boc-Lys(Boc).

### Example 7: bis(bis(4-borono-3-fluorobenzoyl)-3,5-aminomethylbenzoate-epsilon,alpha-Lys-N-beta-Ala-OSu=(S)-3-(2,6-bis(3,5-bis((3-fluoro-4-(4,4,5,5-tetramethyl-1,3,2-doxaborolan-2-yl)benzamido)methyl)benzamido)hexanamido)propanoate

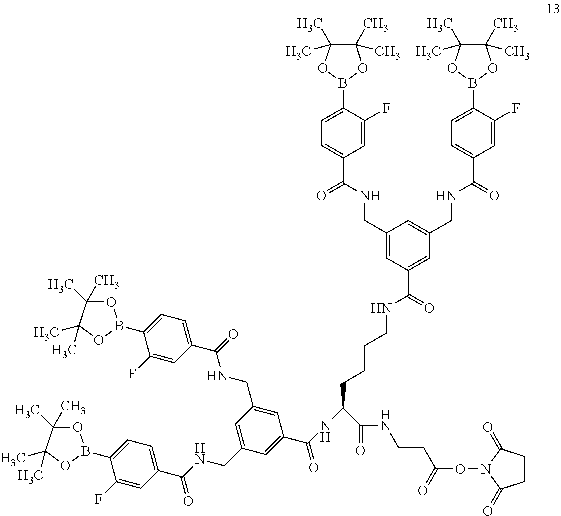 [SMILES: CC1(C)OB(c2ccc(C(=O)NCc3cc(CNC(=O)c4ccc(B5OC(C)(C)C(C)(C)O5)c(F)c4)cc(C(=O)NCCCC[C@H](NC(=O)c4cc(CNC(=O)c5ccc(B6OC(C)(C)C(C)(C)O6)c(F)c5)cc(CNC(=O)c5ccc(B6OC(C)(C)C(C)(C)O6)c(F)c5)c4)C(=O)NCCC(=O)ON4C(=O)CCC4=O)c3)cc2F)OC1(C)C]

3,5-Dimethylbenzoic acid (1, 45.1 g, 18.4 mmol) was suspended in methanol (130 mL) and treated with concentrated sulfuric acid (13 mL). The mixture was refluxed for 2 days. After neutralization with sodium carbonate (80 g), the mixture was dissolved in water (250 mL) and 5 extracted with diethyl ether (2×300 mL). The organic phases were dried over anhydrous sodium sulfate, filtered and evaporated to dryness affording methyl 3,5-dimethylbenzoate (2) as pale yellow oil. Yield: 46.8 g (95%). ¹H NMR spectrum (300 MHz, CDCl₃, δH): 7.67 (s, 2H); 7.19 (s, 1H); 3.91 (s, 3H); 2.37 (s, 6H).

A mixture of the above methyl 3,5-dimethylbenzoate (2, 46.7 g, 284 mmol), N-bromosuccinimide (NBS, 177 g, 994 mmol) and a spatula of azobisisobutyronitrile in methyl formate (550 mL) was irradiated with visible light while heating to reflux for 20 hours. The solvent was evaporated and the residue was dissolved in dichloromethane (300 mL). The precipitated succinimide was filtered off and the filtrate was washed with saturated aqueous solution of sodium sulfite (2×250 mL). The organic layer was dried over anhydrous sodium sulfate, filtered and evaporated. The residue was purified by flash column chromatography (Silicagel 60, 0.040-0.063 mm; eluent:hexane/ethyl acetate 15:1). The product was crystallized from ethyl acetate/cyclohexane mixture (1:5, 360 mL) giving methyl 3,5-bis(bromomethyl)benzoate (3) as white solid. Yield: 46.5 g (51%). RE (SiO2, hexanes/ethyl acetate 9:1): 0.50. ¹H NMR spectrum (300 MHz, CDCl₃, δH): 8.03-7.97 (m, 2H); 7.62 (s, 1H); 4.50 (s, 4H); 3.94 (s, 3H).

A suspension of the above bromide (3, 35.2 g, 109 mmol) and sodium diformylamide (29.1 g, 306 mmol) in dry acetonitrile (200 mL) was refluxed for 4 hours. After removal of a white solid by filtration, the solvent was evaporated. Recrystallization from ethyl acetate/cyclohexane mixture afforded methyl 3,5-bis((N-formylformamido)methyl)benzoate (4) as white powder.

Yield: 32.7 g (98%). ¹H NMR spectrum (300 MHz, DMSO-d6, 6H): 9.08 (s, 4H); 7.72 (s, 2H); 7.44 (s, 1H); 4.70 (s, 4H); 3.84 (s, 3H).

Benzoate (4, 32.7 g, 107 mmol) was dissolved in a mixture of 1,4-dioxane (340 mL) and concentrated hydrochloric acid (430 mL) and heated for 2 hours to reflux. After cooling down to room temperature, a flow of air was passed through the solution. Product began to precipitate. After 1 hour, the solvent was evaporated and product was recrystallized from methanol/diethyl ether mixture (300 mL) affording 3,5-bis(aminomethyl)benzoic acid dihydrochloride (5) as white powder. Yield: 22.2 g (82%). ¹H NMR spectrum (300 MHz, D2O, δH): 8.08 (s, 2H); 7.72 (s, 1H); 4.26 (s, 4H).

Dihydrochloride (5, 6.33 g, 25.0 mmol) was dissolved in water (110 mL). Subsequently N,N-diisopropylethylamine (17.4 mL, 100 mmol), N,N-dimethylformamide (110 mL) and 2,5-dioxopyrrolidin-1-yl 3-fluoro-4-(4,4,5,5-tetramethyl-1,3,2-dioxaborolan-2-yl)benzoate (6, 18.2 g, 50.0 mmol) were added. The mixture was stirred overnight at room temperature; then it was neutralized by 1 M aqueous solution of hydrochloric acid. The solvent was co-evaporated with toluene three times. The residue was dissolved in dichloromethane/toluene mixture (1:1, 100 mL) and treated with pinacol (0.60 g, 5.00 mol). The mixture was evaporated three times from toluene. The residue was dissolved in ethyl acetate (250 mL) and washed with water (3×150 mL). Organic layer was dried over anhydrous sodium sulfate, filtered and evaporated. The residue was dissolved in dichloromethane (50 mL) and product started to precipitate. Cyclohexane was added (170 mL). The precipitate was collected by filtration, washed with cyclohexane and diethyl ether and dried in vacuo to give 3,5-bis((3-fluoro-4-(4,4,5,5-tetramethyl-1,3,2-dioxaborolan-2-yl)benzamido)methyl)benzoic acid (7) as white powder. Yield: 14.5 g (86%). ¹H NMR spectrum (300 MHz, DMSO-d6, δH): 12.96 (bs, 1H); 9.33-9.23 (m, 2H); 7.83-7.67 (m, 6H); 7.64-7.57 (m, 2H); 7.54 (s, 1H); 4.55-4.46 (m, 4H); 1.31 (s, 24H). LC-MS: 512.0 (M+H−2×pinacol)+.

The above acid (7, 14.4 g, 21.3 mmol) was dissolved in acetonitrile/N,N-dimethylformamide mixture (4:1, 100 mL). Subsequently N-hydroxysuccinimide (HOSu, 2.95 g, 25.6 mmol) and N,N-dicyclohexylcarbodiimide (DCC, 5.28 g, 25.6 mmol) were added. The mixture was stirred overnight at room temperature. The insoluble by-product was filtered off and the filtrate was evaporated. The residue was dissolved in ethyl acetate (250 mL) and washed with water (2×150 mL) and brine (1×150 mL). Organic layer was dried over anhydrous sodium sulfate, filtered and evaporated. The residue was dissolved in acetonitrile (100 mL). The residual N,N-dicyclohexylurea was filtered off and the filtrate was evaporated. The residue was dissolve in tetrahydrofuran (150 mL) and treated with pinacol (0.60 g, 5.00 mmol) and molecular sieves overnight. The mixture was filtered through the celite pad and the filtrate was evaporated. The residue was dissolved in dichloromethane (40 mL). The product precipitated by addition of cyclohexane (150 mL). The precipitate was filtered, washed with cyclohexane and diethyl ether and dried in vacuo to give 2,5-dioxopyrrolidin-1-yl 3,5-bis((3-fluoro-4-(4,4,5,5-tetramethyl-1,3,2-dioxaborolan-2-yl)benzamido)methyl)benzoate (8) as white powder. Yield: 13.3 g (75%). ¹H NMR spectrum (300 MHz, DMSO-d6, 6H): 9.34-9.21 (m, 2H); 7.94 (s, 2H); 7.79-7.66 (m, 5H); 7.65-7.56 (m, 2H); 4.62-4.50 (m, 4H); 2.88 (s, 4H); 1.31 (s, 24H). LC-MS: 773.4 (M+H)+, 691.2 (M+H−pinacol)+, 609.1 (M+H−2×pinacol)+.

2-Chlorotrityl resin 100-200 mesh 1.8 mmol/g (9, 10.9 g, 19.7 mmol) was left to swell in dry dichloromethane (140 mL) for 20 minutes. A solution of 3-((((9H-fluoren-9-yl)methoxy)carbonyl)amino)propanoic acid (Fmoc-bAla-OH, 4.08 g, 13.1 mmol) and N,N-diisopropylethylamine (8.68 mL, 49.9 mmol) in dry dichloromethane (120 mL) was added to resin and the mixture was shaken overnight. Resin was filtered and treated with a solution of N,N-diisopropylethylamine (4.57 mL, 26.2 mmol) in methanol/dichloromethane mixture (1:4, 10 min, 140 mL). Then resin was washed with dichloromethane (2×130 mL) and N,N-dimethylformamide (2×130 mL). Fmoc group was removed by treatment with 20% piperidine in N,N-dimethylformamide (1×5 min, 1×1 m, 130 mL). Resin was washed with N,N-dimethylformamide (2×130 mL), 2-propanol (2×130 mL), dichloronethane (2×130 mL) and N,N-dimethylformamide (2×130 mL). Solution of N2,N6-bis(tert-butoxycarbonyl)-L-lysine (Boc-Lys(Boc)-OH, 9.09 g, 26.2 mmol), 5-chloro-1-((dimethylamino)(dimethyliminio)methyl)-1H-benzo[d][1,2,3]triazole 3-oxide tetrafluoroborate (TCTU, 9.33 g, 26.2 mmol) and N,N-diisopropylethylamine (8.23 mL, 47.2 mmol) in N,N-dimethylformamide (110 mL) was added to resin and mixture was shaken for 3 hours. Resin was filtered and washed with N,N-dimethylformamide (2×130 mL) and dichloromethane (10×130 mL). The product was cleaved from resin by treatment with 2,2,2-trifluoroethanol (220 mL) overnight. Resin was filtered off and washed with dichloromethane (2×200 mL). Solutions were combined; solvent was evaporated and the residue was purified by flash column chromatography (Silicagel 60, 0.040-063 mm; eluent:dichloromethane/methanol 90:10) affording (S)-3-(2,6-bis((tert-butoxycarbonyl)amino)hexanamido)propanoic acid (10) as white solid. Yield: 4.30 g (78%). RF (SiO2, dichloromethane/methanol 90:10): 0.40.

¹H NMR spectrum (300 MHz, AcOD-d4, δH): 4.27-3.99 (m, 1H); 3.65-3.44 (m, 2H); 3.17-3.00 (m, 2H); 2.70-2.56 (m, 2H); 1.86-1.58 (m, 2H); 1.57-1.26 (m, 22H). LC-MS: 417.5 (M+H)+.

The above compound (10, 4.30 g, 10.3 mmol) was dissolved in trifluoroacetic acid (50 mL) and left to stay for 1.5 hours. The solvent was evaporated. Diethyl ether (100 mL) was added and mixture was stirred overnight. The solvent was decanted and the residue was dried in vacuo to yield (S)-6-((2-carboxyethyl)amino)-6-oxohexane-1,5-diaminium 2,2,2-trifluoroacetate (11) as tough oil. Yield: 4.50 g (100%). ¹H NMR spectrum (300 MHz, DMSO-d6, δH): 8.58 (t, J=5.4 Hz, 1H); 8.18 (bs, 2H); 7.87 (bs, 2H); 3.77-3.62 (m, 1H); 3.34-3.18 (m, 2H); 2.83-2.65 (m, 2H); 1.74-1.60 (m, 2H); 1.60-1.44 (m, 2H); 1.37-1.19 (m, 2H). LC-MS: 217.2 (M+H)+.

The above salt (11, 2.70 g, 6.06 mmol) was dissolved in N,N-dimethylformamide (100 mL). Subsequently N,N-diisopropylethylamine (5.30 mL, 30.3 mmol), water (50 mL) and activated ester (8, 9.36 g, 12.1 mmol) were added. The mixture was stirred overnight at room temperature; then it was neutralized by 1 M aqueous solution of hydrochloric acid. The solvent was co-evaporated with toluene three times. The residue was dissolved in dichloromethane/toluene mixture (1:1, 100 mL) and treated with pinacol (0.50 g, 4.23 mmol). The mixture was evaporated three times from toluene. The residue was dissolved in ethyl acetate (250 mL) and washed with water (1×100 mL) and brine (1×100 mL). Organic layer was dried over anhydrous sodium sulfate, filtered and evaporated. Partial pinacol ester cleavage was observed by NMR analysis. The material was treated with pinacol (0.04 g, 0.34 mmol) and magnesium sulfate (20.0 g) in tetrahydrofuran (110 mL) overnight. The mixture was filtered and the filtrate was evaporated. The product was crystallized from dichloromethane/cyclohexane mixture (1:5, 180 mL) affording 3,5-bis((3-fluoro-4-(4,4,5,5-tetramethyl-1,3,2-dioxaborolan-2-yl)benzamido)methyl)benzamido)hexanamido)propanoic acid (12) as pale brown powder. Yield: 5.86 g (63%). ¹H NMR spectrum (300 MHz, DMSO-d6, δH): 9.31-9.13 (m, 4H); 8.53-8.43 (m, 1H); 8.43-8.35 (m, 1H); 8.11-7.98 (m, 1H); 7.78-7.55 (m, 16H); 7.48-7.38 (m, 2H); 4.55-4.43 (m, 8H); 4.43-4.33 (m, 1H); 3.31-3.13 (m, 4H); 2.38 (t, J=6.4 Hz, 2H); 1.79-1.64 (m, 2H); 1.57-1.44 (m, 2H); 1.42-1.21 (m, 50H).

The carboxylic acid (12, 5.46 g, 3.57 mmol) was dissolved in acetonitrile (50 mL). N-Hydroxysuccinimide (HOSu, 0.70 g, 6.07 mmol) and N,N-dicyclohexylcarbodiimide (1.47 g, 7.14 mmol) were added. Resulting mixture was stirred overnight at room temperature. The byproduct was removed by filtration. The filtrate was evaporated. The residue was dissolved in ethyl acetate (150 mL) and washed with water (1×100 mL) and brine (1×100 mL). The organic layer was dried over anhydrous sodium sulfate, filtered and evaporated. The residue was dissolved in dichloromethane (60 mL) and treated with pinacol (0.06 g, 0.50 mmol) and molecular sieves overnight. The mixture was filtered and filtrate was evaporated. The residue was dissolved in ethyl acetate (10 mL) and precipitated after addition of diethyl ether (90 ml). The product was collected by filtration, washed with diethyl ether and dried in vacuo to yield the title compound (13) as pale brown powder. Product contains traces of N,N-dicyclohexylurea.

Yield: 1.55 g (27%). ¹H NMR spectrum (300 MHz, DMSO-d6, δH): 9.28-9.17 (m, 3H); 8.52-8.33 (m, 2H); 8.25-8.15 (m, 1H); 7.80-7.51 (m, 16H); 7.48-7.35 (m, 2H); 4.58-4.32 (m, 9H); 3.49-3.35 (m, 2H); 3.25-3.09 (m, 2H); 2.91-2.72 (m, 6H); 1.81-1.65 (m, 2H); 1.57-1.42 (m, 2H); 1.41-1.12 (m, 50H). LC-MS: 1631.9 (M+H)+, 1549.0 (M−pinacol+H)+, 715.0 (M−2×H2O−2×pinacol/2+H)+, 1384.5 (M−3×pinacol+H)+, 1302.3 (M−4×pinacol+H)+.

### Example 8: (7S,18S)-18-(3-((S)-2,6-bis(3-Fluoro-4-(4,4,5,5-tetramethyl-1,3,2-dioxaborolan-2-yl)benzamido)hexanamido)propanamido)-7-(3-fluoro-4-(4,4,5,5-tetramethyl-1,3,2-dioxaborolan-2-yl)benzamido)-1-(3-fluoro-4-(4,4,5,5-tetramethyl-1,3,2-dioxaborolan-2-yl)phenyl)-1,8,12,19-tetraoxo-2,9,13,20-tetaazatricosan-23-oic acid

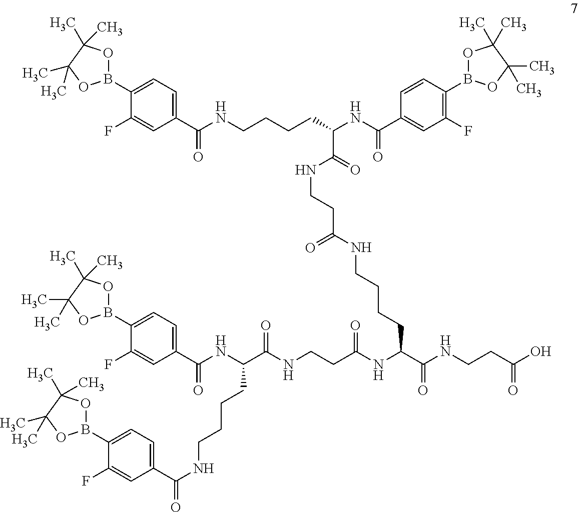 [SMILES: CC1(C)OB(c2ccc(C(=O)NCCCC[C@H](NC(=O)c3ccc(B4OC(C)(C)C(C)(C)O4)c(F)c3)C(=O)NCCC(=O)NCCCC[C@H](NC(=O)CCNC(=O)[C@H](CCCCNC(=O)c3ccc(B4OC(C)(C)C(C)(C)O4)c(F)c3)NC(=O)c3ccc(B4OC(C)(C)C(C)(C)O4)c(F)c3)C(=O)NCCC(=O)O)cc2F)OC1(C)C]

Mixture of 2-fluoro-4-carboxyphenylboronic acid (1, 15.1 g, 82.0 mmol), pinacol (9.81 g, 83.0 mmol) and magnesium sulfate (150 g) in tetrahydrofuran (400 mL) was stirred over weekend at room temperature. The suspension was filtered through celite pad, the filtrate was evaporated and dried in vacuo to yield 3-fluoro-4-(4,4,5,5-tetramethyl-1,3,2-dioxaborolan-2-yl)benzoic acid (2) as pale yellow powder. Yield: 21.5 g (98%). ¹H NMR spectrum (400 MHz, DMSO-d6, δH): 7.95-7.42 (m, 3H); 1.30 (s, 12H).

The carboxylic acid (2, 21.4 g, 81.9 mmol) was dissolved in dichloromethane (300 mL). N-Hydroxysuccinimide (HOSu, 18.8 g, 163 mmol) and N-(3-dimethylaminopropyl)-N′ethylcarbodiimide hydrochloride (EDC.HCl, 31.3 g, 163 mmol) were added. Resulting mixture was stirred overnight at room temperature. The reaction mixture was washed with 0.5 M aqueous solution of hydrochloric acid (1×200 mL), water (1×200 mL) and brine (1×200 mL), dried over anhydrous sodium sulfate, filtered and evaporated. The residue was dissolved in dichloromethane (60 mL) and precipitated by addition of cyclohexane (250 mL). The product was collected by filtration, washed with cyclohexane and dried in vacuo to yield 2,5-dioxopyrrolidin-1-yl 3-fluoro-4-(4,4,5,5-tetramethyl-1,3,2-dioxaborolan-2-yl)benzoate (3) as beige powder. Yield: 27.8 g (93%). ¹H NMR spectrum (400 MHz, DMSO-d6, δH): 7.98-7.87 (m, 2H); 7.80 (dd, J=9.2 Hz, 1H); 2.90 (s, 4H); 1.33 (s, 12H).

2-Chlorotrityl resin 100-200 mesh 1.8 mmol/g (4, 16.4 g, 29.5 mmol) was left to swell in dry dichloromethane (230 mL) for 20 minutes. A solution of 3-((((9H-fluoren-9-yl)methoxy)carbonyl)amino)propanoic acid (Fmoc-bAla-OH, 6.13 g, 19.7 mmol) and N,N-diisopropylethylamine (13.0 mL, 74.8 mmol) in dry dichloromethane (180 mL) was added to resin and the mixture was shaken overnight. Resin was filtered and treated with a solution of N,N-diisopropylethylamine (6.86 mL, 39.4 mmol) in methanol/dichloromethane mixture (1:4, 10 min, 200 mL). Then resin was washed with dichloromethane (2×200 mL) and N,N-dimethylformamide (2×200 mL). Fmoc group was removed by treatment with 20% piperidine in N,N-dimethylformamide (1×5 min, 1×15 min, 2×200 mL). Resin was washed with N,N-dimethylformamide (2×200 mL), 2-propanol (2×200 mL), dichloromethane (2×200 mL) and N,N-dimethylformamide (2×200 mL). Solution of N2,N6-bis(((9H-fluoren-9-yl)methoxy)carbonyl)-L-lysine (Fmoc-Lys(Fmoc)-OH, 23.3 g, 39.4 mmol), 5-chloro-1-((dimethylamino)(dimethyliminio)methyl)-1H-benzo[d][1,2,3]triazole 3-oxide tetrafluoroborate (TCTU, 14.0 g, 39.4 mmol) and N,N-diisopropylethylamine (12.3 mL, 70.9 mmol) in N,N-dimethylformamide (180 mL) was added to resin and mixture was shaken for 2.5 hours. Resin was filtered and washed with N,N-dimethylformamide (2×200 mL), dichloromethane (2×200 mL) and N,N-dimethylformamide (2×200 mL). Fmoc group was removed by treatment with 20% piperidine in N,N-dimethylformamide (1×5 min, 1×15 min, 2×200 mL). Resin was washed with N,N-dimethylformamide (2×200 mL), 2-propanol (2×200 mL), dichloromethane (2×200 mL) and N,N-dimethylformamide (2×200 mL). Solution of 3-((((9H-fluoren-9-yl)methoxy)carbonyl)amino)propanoic acid (Fmoc-bAla-OH, 24.5 g, 78.7 mmol), 5-chloro-1-((dimethylamino)(dimethyliminio)methyl)-1H-benzo[d][1,2,3]triazole 3-oxide tetrafluoroborate (TCTU, 28.0 g, 78.7 mmol) and N,N-diisopropylethylamine (24.7 mL, 142 mmol) in N,N-dimethylformamide (230 mL) was added to resin and mixture was shaken for 3 hours. Resin was filtered and washed with N,N-dimethylformamide (2×200 mL), dichloromethane (2×200 mL) and N,N-dimethylformamide (2×200 mL). Fmoc group was removed by treatment with 20% piperidine in N,N-dimethylformamide (1×5 min, 1×15 min, 2×200 mL). Resin was washed with N,N-dimethylformamide (2×200 mL), 2-propanol (2×200 mL), dichloromethane (2×200 mL) and N,N-dimethylformamide (2×200 mL). Solution of N2,N6-bis(tert-butoxycarbonyl)-L-lysine (Boc-Lys(Boc)-OH, 27.3 g, 78.7 mmol), 5-chloro-1-((dimethylamino)(dimethyliminio)methyl)-1H-benzo[d][1,2,3]triazole 3-oxide tetrafluoroborate (TCTU, 28.0 g, 78.7 mmol) and N,N-diisopropylethylamine (24.7 mL, 142 mmol) in N,N-dimethylformamide (230 mL) was added to resin and mixture was shaken for 3 hours. Resin was filtered and washed with N,N-dimethylformamide (2×200 mL), dichloromethane (2×200 mL) and N,N-dimethylformamide (2×200 mL). Fmoc group was removed by treatment with 20% piperidine in N,N-dimethylformamide (1×5 min, 1×15 min, 2×200 mL). Resin was washed with N,N-dimethylformamide (2×200 mL), 2-propanol (2×200 mL), dichloromethane (2×200 mL) and N,N-dimethylformamide (2×200 mL). The product was cleaved from resin by treatment with 2,2,2-trifluoroethanol (350 mL) overnight. Resin was filtered off and washed with dichloromethane (2×300 mL). Solutions were combined; solvent was evaporated and the residue was purified by flash column chromatography (Silicagel 60, 0.040-063 mm; eluent:dichloromethane/methanol 85:15) affording (10S,21S)-21-(3-((S)-2,6-bis((tert-butoxycarbonyl)amino)hexanamido)propanamido)-10-((tert-butoxycarbonyl)amino)-2,2-dimethyl-4,11,15,22-tetraoxo-3-oxa-5,12,16,23-tetraazahexacosan-26-oic acid (5) as white solid. Yield: 11.3 g (56%). ¹H NMR spectrum (300 MHz, AcOD-d4, δH): 4.52-4.43 (m, 1H); 4.22-3.98 (m, 2H); 3.64-3.44 (m, 6H); 3.27-3.16 (m, 2H); 3.15-3.03 (m, 4H); 2.69-2.48 (m, 6H); 1.84-1.59 (m, 6H); 1.58-1.28 (m, 48H). LC-MS: 1016.2 (M+H)+.

The above compound (5, 11.3 g, 11.1 mmol) was dissolved in trifluoroacetic acid (200 mL) and left to stand for 1.5 hours. Then the mixture was concentrated and diethyl ether (200 mL) was added. After overnight stirring the precipitate was filtered, washed with diethyl ether and dried in vacuo to yield (5S,12S,23S)-12-((2-carboxyethyl)carbamoyl)-6,10,18,22-tetraoxo-7,11,17,21-tetraazaheptacosane-1,5,23,27-tetraaminium 2,2,2-trifluoroacetate (6) as white powder. Yield: 9.25 g (99%). ¹H NMR spectrum (300 MHz, DMSO-d6, δH): 8.56-8.44 (m, 2H); 8.27-7.72 (m, 11H); 4.22-4.08 (m, 1H); 3.78-3.60 (m, 2H); 3.39-3.17 (m, 6H); 3.07-2.92 (m, 2H); 2.82-2.66 (m, 4H); 2.42-2.19 (m, 6H); 1.77-1.43 (m, 10H); 1.42-1.14 (m, 8H).

The above salt (6, 7.91 g, 9.37 mmol) was dissolved in N,N-dimethylformamide (170 mL). Subsequently N,N-diisopropylethylamine (14.7 mL, 84.3 mmol), water (0.50 mL) and activated ester (3, 13.6 g, 37.5 mmol) were added. The mixture was stirred overnight at room temperature; then it was acidified by 1 M aqueous solution of hydrochloric acid. The solvent was co-evaporated with toluene three times. The residue was dissolved in dichloromethane/toluene mixture (1:1, 100 mL) and treated with pinacol (1.00 g, 8.46 mmol). The mixture was evaporated three times from toluene. The residue was dissolved in ethyl acetate (150 mL) and washed with water (1×100 mL) and brine (1×100 mL). Organic layer was dried over anhydrous sodium sulfate, filtered and concentrated to ⅓ of volume. Cyclohexane (150 mL) was added; the precipitate was filtered and washed with cyclohexane. The solid was suspended in acetonitrile/diethyl ether mixture (1:1, 150 mL). The precipitate was filtered, washed with acetonitrile and dried in vacuo to yield the title compound (7) as white solid. Yield: 4.10 g (27%).

¹H NMR spectrum (300 MHz, DMSO-d6, δH): 8.67-8.43 (m, 4H); 8.05-7.83 (m, 4H); 7.82-7.47 (m, 13H); 4.46-4.27 (m, 2H); 4.20-4.05 (m, 1H); 3.42-3.13 (m, 10H); 3.05-2.90 (m, 2H); 2.42-2.27 (m, 4H); 2.27-2.17 (m, 2H); 1.84-1.66 (m, 4H); 1.63-1.10 (m, 62H). LC-MS: 1226.4 (M−3×H2O−4×pinacol+H)+.

### Example 9: 2,5-Dioxopyrrolidin-1-yl N-(2-(3-fluoro-4-(4,4,5,5-tetramethyl-1,3,2-dioxaborolan-2-yl)benzamido)ethyl)-N-(3-fluoro-4-(4,4,5,5-tetramethyl-1,3,2-dioxaborolan-2-yl)benzoyl)glycinate

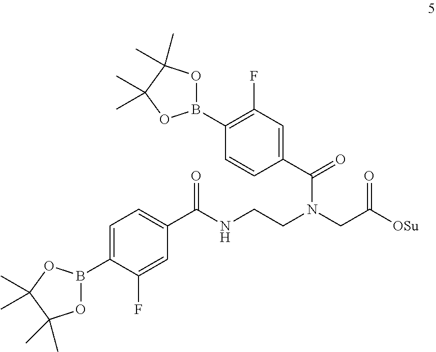

3-Fluoro-4-(4,4,5,5-tetramethyl-1,3,2-dioxaborolan-2-yl)benzoic acid (2, 8.85 g, 33.3 mmol) was dissolved in dichloromethane (100 mL) followed by addition of 1-((dimethylamino)(dimethyliminio)methyl)-1H-[1,2,3]triazolo[4,5-b]pyridine 3-oxide hexafluorophosphate(V) (HATU, 12.3 g, 32.4 mmol), N,N-diisopropylethylamine (14.5 mL, 83.2 mmol) and tert-butyl (2-aminoethyl)glycinate hydrochloride (1, 4.11 g, 16.6 mmol). The reaction mixture was allowed to stir for 18 hours at ambient temperature. The reaction mixture was extracted with 1 M aqueous solution of hydrochloric acid (2×100 mL), water (1×100 mL) and brine (1×100 mL). The organic layer was dried over anhydrous sodium sulfate, filtered and evaporated. The crude product was dissolved in dry tetrahydrofuran (50 mL) and 2,3-dimethyl-2,3-butanediol (3.70 g, 31.5 mmol) was added. Reaction mixture was allowed to stir overnight at room temperature. The reaction mixture was then evaporated and the crude product was purified by flash chromatography (Silicagel 60, 0.063-0.200 mm; eluent:dichloromethane/ethyl acetate 5:2) to provide tert-butyl N-(2-(3-fluoro-4-(4,4,5,5-tetramethyl-1,3,2-dioxaborolan-2-yl)benzamido)ethyl)-N-(3-fluoro-4-(4,4,5,5-tetramethyl-1,3,2-dioxaborolan-2-yl)benzoyl)glycinate (3) as white foam. Yield: 8.13 g (73%). ¹H NMR spectrum (300 MHz, DMSO-d6, δH): 8.78-8.55 (m, 1H); 7.79-7.44 (m, 4H); 7.12-6.88 (m, 2H); 4.18-3.90 (m, 2H); 3.67-3.47 (m, 2H); 3.45-3.29 (m, 2H); 1.44 (s, 9H); 1.30 (s, 24H).

The above prepared compound (3, 8.13 g, 12.1 mmol) was dissolved in trifluoroacetic acid (100 mL) and left to stay for 2.5 hours. Then the solvent was evaporated and co-evaporated with toluene twice. The residue was dissolved in dichloromethane (30 mL) and cyclohexane (250 mL) was added. The product was collected by filtration, washed with cyclohexane and dried in vacuo to yield N-(2-(3-fluoro-4-(4,4,5,5-tetramethyl-1,3,2-dioxaborolan-2-yl)benzamido)ethyl)-N-(3-fluoro-4-(4,4,5,5-tetramethyl-1,3,2-dioxaborolan-2-yl)benzoyl)glycine (4) as white powder. Yield: 6.91 g (93%). ¹H NMR spectrum (300 MHz, DMSO-d6, 80 C, δH): 8.49-8.38 (m, 1H); 7.76-7.68 (m, 1H); 7.67-7.59 (m, 2H); 7.57-7.45 (m, 1H); 7.16-7.09 (m, 1H); 7.04-6.94 (m, 1H); 4.20-4.03 (m, 2H); 3.59-3.40 (m, 4H); 1.33 (s, 24H). LC-MS: 449.9 (M−2×pinacol+H)+, 532.1 (M−pinacol+H)+, 614.2 (M+H)+.

The acid (4, 6.90 g, 11.2 mmol) was dissolved in dichloromethane/tetrahydrofuran mixture (1:1, 100 mL) followed by addition of N-hydroxysuccinimide (1.36 g, 11.8 mmol) and N-(3-dimethylaminopropyl)-N-ethylcarbodiimide hydrochloride (2.26 g, 11.8 mmol). The mixture was stirred overnight at room temperature. The solvent was evaporated. The residue was dissolved in ethyl acetate (150 mL) and washed with water (2×100 mL) and brine (1×100 mL). The organic layer was dried over anhydrous sodium sulfate, filtered and evaporated. The product precipitated from dichloromethane/cyclohexane mixture (25 mL/250 mL). The precipitate was collected by filtration, washed with cyclohexane and dried in vacuo to yield the title compound (5) as white powder. Yield: 7.62 g (96%). ¹H NMR spectrum (300 MHz, DMSO-d6, 80 C, δH): 8.51-8.38 (m, 1H); 7.77-7.57 (m, 3H); 7.55-7.45 (m, 1H); 7.18-7.10 (m, 1H); 7.06-6.97 (m, 1H); 4.62 (bs, 2H); 3.67-3.41 (m, 4H); 2.84 (s, 4H); 1.33 (s, 24H). LC-MS: 547.0 (M−2×pinacol+H)+, 629.1 (M−pinacol+H)+, 711.3 (M+H)+.

### Example 10: N-(6-Fluoro-1-hydroxy-1,3-dihydrobenzo[c][1,2]oxaborole-5-carbonyl)-N-(2-(6-fluoro-1-hydroxy-1,3-dihydrobenzo[c][1,2]oxaborole-5-carboxamido)ethyl)glycine

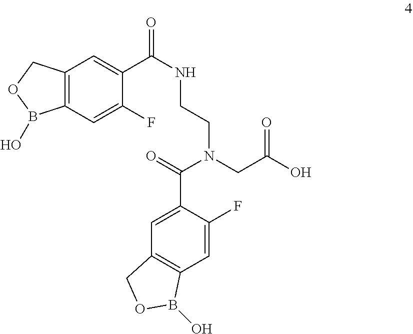 [SMILES: O=C(O)CN(CCNC(=O)c1cc2c(cc1F)B(O)OC2)C(=O)c1cc2c(cc1F)B(O)OC2]

6-Fluoro-1-hydroxy-1,3-dihydrobenzo[c][1,2]oxaborole-5-carboxylic acid (1, 10.0 g, 51.0 mmol) was dissolved in tetrahydrofuran (100 mL). N,N-Dimethylformamide (15 mL), N-hydroxysuccinimide (6.46 g, 56.1 mmol) and N-(3-dimethylaminopropyl)-N-ethylcarbodiimide hydrochloride (10.8 g, 56.1 mmol) were added at room temperature. After stirring for 2 hours, volatiles were evaporated under reduced pressure and the residue was redissolved in ethyl acetate (400 mL) and washed with 1 M aqueous hydrochloric acid (2×100 mL). Organic portion was dried over anhydrous sodium sulfate. Volatiles were evaporated under reduced pressure to give 2,5-dioxopyrrolidin-1-yl 6-fluoro-1-hydroxy-1,3-dihydrobenzo[c][1,2]oxaborole-5-carboxylate (2) as a white solid. Yield: 13.8 g (92%).

¹H NMR spectrum (300 MHz, DMSO-d6, δH): 9.65 (s, 1H); 8.11 (d, J=5.9 Hz, 1H); 7.71 (d, J=10.1 Hz, 1H); 5.08 (s, 2H); 2.90 (s, 4H). LC-MS: 294.4 (M+H)+.

(2-Aminoethyl)glycine (3, 1.81 g, 15.4 mmol) was dissolved in N,N-dimethylformamide (40 mL), triethylamine (12.8 mL, 92.1 mmol) and 2,5-dioxopyrrolidin-1-yl 6-fluoro-1-hydroxy-1,3-dihydrobenzo[c][1,2]oxaborole-5-carboxylate (2, 9.00 g, 30.7 mmol) were added at room temperature. After stirring for 16 hours at room temperature, reaction mixture was heated to 40° C. and stirred for another 72 hours. Volatiles were then evaporated under reduced pressure and the residue was redissolved in ethyl acetate (400 mL) and washed with 1 M aqueous hydrochloric acid (100 mL). Organic portion was dried over anhydrous sodium sulfate. Volatiles were evaporated under reduced pressure and product was precipitated from acetonitrile/water mixture, collected by centrifuge and freeze-dried to afford N-(6-fluoro-1-hydroxy-1,3-dihydrobenzo[c][1,2]oxaborole-5-carbonyl)-N-(2-(6-fluoro-1-hydroxy-1,3-dihydrobenzo[c][1,2]oxaborole-5-carboxamido)ethyl)glycine 4 as off-white solid.

Yield: 1.99 g (27%). ¹H NMR spectrum (300 MHz, DMSO-d6, δH): 12.87 (bs, 1H); 9.50-9.37 (m, 2H); 8.52-8.22 (m, 1H); 7.69-7.30 (m, 4H); 5.08-4.70 (m, 4H); 4.27-3.96 (m, 2H); 3.74-3.35 (m, 4H). LC-MS: 475.5 (M+H)+.

### Example 11: 2,5-Diexopyrrolidin-1-yl (S)-3-(2,6-bis(3,5-bis((4-(4,4,5,5-tetramethyl-1,3,2-dioxaborolan-2-yl)benzamido)methyl)benzamido)hexanamido)propanoate

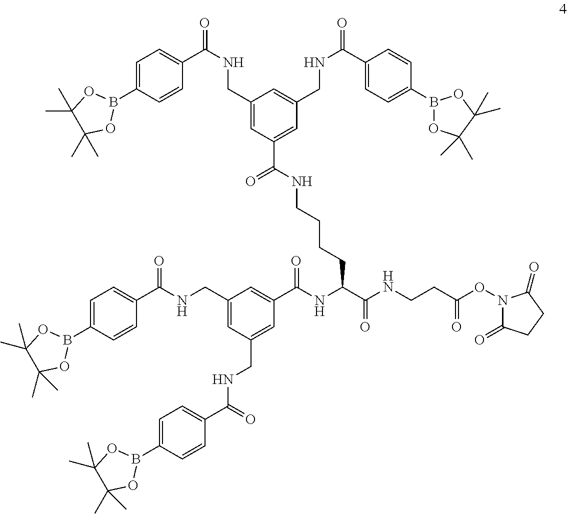 [SMILES: CC1(C)OB(c2ccc(C(=O)NCc3cc(CNC(=O)c4ccc(B5OC(C)(C)C(C)(C)O5)cc4)cc(C(=O)NCCCC[C@H](NC(=O)c4cc(CNC(=O)c5ccc(B6OC(C)(C)C(C)(C)O6)cc5)cc(CNC(=O)c5ccc(B6OC(C)(C)C(C)(C)O6)cc5)c4)C(=O)NCCC(=O)ON4C(=O)CCC4=O)c3)cc2)OC1(C)C]

2-Chlorotrityl resin 100-200 mesh 1.5 mmol/g (1, 21.0 g, 31.5 mmol) was left to swell in dry dichloromethane (300 mL) for 20 minutes. A solution of 3-((((9H-fluoren-9-yl)methoxy)carbonyl)amino)propanoic acid (Fmoc-bAla-OH, 6.54 g, 21.0 mmol) and N,N-diisopropylethylamine (13.9 mL, 79.8 mmol) in dry dichloromethane (250 mL) was added to resin and the mixture was shaken over the weekend. Resin was filtered and treated with a solution of N,N-diisopropylethylamine (7.32 mL, 42.0 mmol) in methanol/dichloromethane mixture (1:4, 1×15 min, 250 mL). Then resin was washed with dichloromethane (2×250 mL) and N,N-dimethylformamide (2×250 mL). Fmoc group was removed by treatment with 20% piperidine in N,N-dimethylformamide (1×10 min, 1×20 min, 2×250 mL). Resin was washed with N,N-dimethylformamide (2×250 mL), 2-propanol (2×250 mL), dichloromethane (2×250 mL) and N,N-dimethylformamide (2×250 mL). Solution of N2,N6-bis(((9H-fluoren-9-yl)methoxy)carbonyl)-L-lysine (Fmoc-Lys(Fmoc)-OH, 18.6 g, 31.5 mmol), 5-chloro-1-((dimethylamino)(dimethyliminio)methyl)-1H-benzo[d][1,2,3]triazole 3-oxide tetrafluoroborate (TCTU, 11.2 g, 31.5 mmol) and N,N-diisopropylethylamine (9.87 mL, 56.7 mmol) in N,N-dimethylformamide (250 mL) was added to resin and mixture was shaken overnight. Resin was filtered and washed with N,N-dimethylformamide (2×250 mL) and dichloromethane (3×250 mL).

Part of resin was removed (2.00 mmol). Fmoc group was removed by treatment with 20% piperidine in N,N-dimethylformamide (1×5 min, 1×10 min, 1×30 min, 3×30 mL). Resin was washed with N,N-dimethylformamide (4×30 mL), dichloromethane (4×30 mL) and N,N-dimethylformamide (4×30 mL). 2,5-Dioxopyrrolidin-1-yl 3,5-bis((4-(4,4,5,5-tetramethyl-1,3,2-dioxaborolan-2-yl)benzamido)methyl)benzoate (2, 3.65 g, 4.72 mmol) and N,N-diisopropylethylamine (1.40 mL, 8.00 mmol) in N,N-dimethylformamide (30 mL) was added to resin and mixture was shaken overnight. Resin was filtered and washed with N,N-dimethylformamide (4×30 mL), dichloromethane (4×30 mL), N,N-dimethylformamide (4×30 mL) and dichloromethane (10×30 mL).

The product was cleaved from resin by treatment with 1,1,1,3,3,3-hexafluoro-2-propanol/dichloromethane mixture (1:2, 30 mL) for 2 hours. Resin was filtered off and washed with dichloromethane (3×30 mL). Solutions were combined and solvent was evaporated. The residue was dissolved in dichloromethane (5 mL) and precipitated after addition of cyclohexane (25 mL). The product was collected by filtration, washed with cyclohexane and dried in vacuo to give (S)-3-(2,6-bis(3,5-bis((4-(4,4,5,5-tetramethyl-1,3,2-dioxaborolan-2-yl)benzamido)methyl)benzamido)hexanamido)propanoic acid (3). Yield: 1.53 g (52%).

¹H NMR spectrum (300 MHz, DMSO-d6, δH): 9.20-9.03 (m, 4H); 8.52-8.42 (m, 1H); 8.39-8.32 (m, 1H); 8.06-7.99 (m, 1H); 7.94-7.80 (m, 10H); 7.78-7.65 (m, 10H); 7.48-7.39 (m, 2H); 4.56-4.43 (m, 8H); 4.43-4.32 (m, 1H); 3.27-3.14 (m, 4H); 2.40-2.29 (m, 2H); 1.78-1.64 (m, 2H); 1.56-1.430 (m, 3H) 1.37-1.21 (s, 49H).

The carboxylic acid (3, 1.53 g, 1.00 mmol) was dissolved in dichloromethane (40 mL). N-Hydroxysuccinimide (HOSu, 148 mg, 1.30 mmol) and N-(3-dimethylaminopropyl)-N-ethylcarbodiimide hydrochloride (EDC.HCl, 242 mg, 1.30 mmol) were added. Resulting mixture was stirred overnight at room temperature. The solvent was evaporated. The residue was dissolved in ethyl acetate (100 mL) and washed with water (2×50 mL). The organic layer was dried over anhydrous sodium sulfate, filtered and evaporated. The residue was dissolved in dichloromethane (10 mL) and precipitated after addition of cyclohexane (50 ml). The product was collected by filtration, washed with cyclohexane and diethyl ether and dried in vacuo to yield the title compound (4) as white powder. Yield: 1.16 g (71%). ¹H NMR spectrum (300 MHz, DMSO-d6, δH): 9.23-9.01 (m, 4H); 8.50-8.42 (m, 1H); 8.41-8.35 (m, 1H); 8.23-8.16 (m, 1H); 7.91-7.81 (m, 9H); 7.77-7.70 (m, 9H); 7.70-7.64 (m, 2H); 7.47-7.40 (m, 2H); 4.55-4.43 (m, 8H); 4.40-4.34 (m, 1H) 3.50-3.38 (m, 2H); 3.26-3.12 (m, 2H); 2.88-2.77 (m, 6H); 1.82-1.63 (m, 2H); 1.60-1.43 (m, 4H); 1.31 (s, 48H). LC-MS: 1631.9 (M+H)+, 1549.0 (M−pinacol+H)+, 715.0 (M−2×H2O−2×pinacol/2+H)+, 1384.5 (M−3×pinacol+H)+, 1302.3 (M−4×pinacol+H)+.

### Example 12: (S)-3-(2,6-Bis(3,5-bis((2-fluoro-4-(4,4,5,5-tetramethyl-1,3,2-dioxaborolan-2-yl)benzamido)methyl)benzamido)hexanamide)propanoic acid

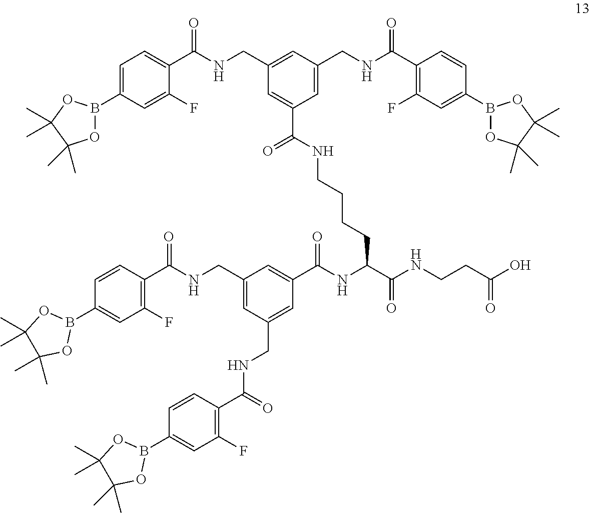

3,5-Dimethylbenzoic acid (1, 300 g, 2.00 mol) was suspended in methanol (900 mL) and treated with concentrated sulfuric acid (90 mL). The mixture was stirred for 3 days. After neutralization with sodium carbonate (480 g) the solvent was evaporated. The residue was dissolved in water (1 L) and extracted with diethyl ether (3×1 L). The organic phases were dried over anhydrous sodium sulfate, filtered and evaporated to dryness affording methyl 3,5-dimethylbenzoate (2) as pale yellow oil. Yield: 309 g (94%). ¹H NMR spectrum (300 MHz, CDCl₃, δH): 7.65 (s, 2H); 7.16 (s, 1H); 3.88 (s, 3H); 2.34 (s, 6H).

A mixture of the above methyl 3,5-dimethylbenzoate (2, 307 g, 1.87 mol), N-bromosuccinimide (1.17 kg, 6.55 mol) and a spatula of azobisisobutyronitrile in methyl formate (2.7 L) was irradiated with visible light while heating to reflux for 20 hours. The solvent was evaporated and the residue was dissolved in dichloromethane (2 L). The precipitated succinimide was filtered off and the filtrate was washed with saturated aqueous solution of sodium sulfite (2×1 L). The organic layer was dried over anhydrous sodium sulfate, filtered and evaporated. Multiple crystallizations from hot ethyl acetate/cyclohexane mixture and washing with cyclohexane gave methyl 3,5-bis(bromomethyl)benzoate (3) as white solid. The product was prepared in two batches. Yield: 243 g (40%). RF (SiO2, hexanes/ethyl acetate 9:1): 0.50. ¹H NMR spectrum (300 MHz, CDCl₃, δH): 8.00 (s, 2H); 7.62 (s, 1H); 4.51 (s, 4H); 3.94 (s, 3H).

A suspension of the above bromide (3, 122 g, 380 mmol) and sodium diformylamide (101 g, 1.06 mol) in dry acetonitrile (900 mL) was refluxed for 4 hours. After removal of a white solid by filtration, the solvent was co-evaporated with ethyl acetate and dried in vacuo to yield methyl 3,5-bis((N-formylformamido)methyl)benzoate (4) as pale yellow solid. Yield: 116 g (100%). ¹H NMR spectrum (300 MHz, DMSO-d6, δH): 9.07 (s, 4H); 7.72 (s, 2H); 7.43 (s, 1H); 4.70 (s, 4H); 3.82 (s, 3H).

Benzoate (4, 116 g, 380 mmol) was dissolved in a mixture of 1,4-dioxane (400 mL) and concentrated hydrochloric acid (600 mL) and heated for 3 hours to reflux. After cooling down to room temperature, a flow of air was passed through the solution. Product began to precipitate. After 1 hour, the solvent was evaporated and product was recrystallized from methanol/diethyl ether mixture affording 3,5-bis(aminomethyl)benzoic acid dihydrochloride (5) as white powder. Yield: 89.5 g (92%). ¹H NMR spectrum (300 MHz, D2O, δH): 8.10 (s, 2H); 7.74 (s, 1H); 4.28 (s, 4H).

Dihydrochloride (5, 30.0 g, 118 mmol) and sodium hydroxide (14.2 g, 356 mmol) were dissolved in water (240 mL). Di-tert-butyl dicarbonate (77.6 g, 356 mmol) in 1,4-dioxane (480 mL) was added with stirring. The reaction mixture was stirred overnight and then diluted with ethyl acetate (400 mL) and 0.5 M aqueous solution of hydrochloric acid (400 mL). Layers were separated and the organic layer was washed with water (2×350 mL), dried over anhydrous sodium sulfate and evaporated. The residue was dissolved in hot ethyl acetate (100 mL) and cyclohexane (400 mL) was added. The precipitate was collected by filtration and washed with cyclohexane to give 3,5-bis(((tert-butoxycarbonyl)amino)methyl)benzoic acid (6) as white solid. Yield: 39.1 g (87%). ¹H NMR spectrum (300 MHz, DMSO-d6, 5H): 7.70 (s, 2H); 7.45-7.36 (m, 2H); 7.33 (s, 1H); 4.21-4.04 (m, 4H); 1.39 (s, 18H).

2-Chlorotrityl chloride resin 100-200 mesh 1.5 mmol/g (7, 21.2 g, 31.8 mmol) was left to swell in dry dichloromethane (280 mL) for 40 minutes. A solution of 3-((((9H-fluoren-9-yl)methoxy)carbonyl)amino)propanoic acid (Fmoc-Ala-OH, 6.61 g, 21.2 mmol) and N,N-diisopropylethylamine (14.1 mL, 80.7 mmol) in dry dichloromethane (220 mL) was added to resin and the mixture was shaken overnight. Resin was filtered and treated with a solution of N,N-diisopropylethylamine (7.40 mL, 42.5 mmol) in methanol/dichloromethane mixture (1:4, 1×20 min, 1×250 mL). Then resin was washed with dichloromethane (2×250 mL) and N,N-dimethylformamide (2×250 mL). Fmoc group was removed by treatment with 20% piperidine in N,N-dimethylformamide (1×5 min, 1×20 min, 2×220 mL). Resin was washed with N,N-dimethylformamide (2×250 mL), 2-propanol (2×250 mL), dichloromethane (2×250 mL) and N,N-dimethylformamide (2×250 mL). Solution of N2,N6-bis(((9H-fluoren-9-yl)methoxy)carbonyl)-L-lysine (Fmoc-Lys(Fmoc)-OH, 18.8 g, 31.8 mmol), 5-chloro-1-((dimethylamino)(dimethyliminio)methyl)-1H-benzo[d][1,2,3]triazole 3-oxide tetrafluoroborate (TCTU, 11.3 g, 31.8 mmol) and N,N-diisopropylethylamine (9.98 mL, 57.3 mmol) in N,N-dimethylformamide (220 mL) was added to resin and mixture was shaken for 2.5 hours. Resin was washed with N,N-dimethylformamide (2×250 mL), dichloromethane (2×250 mL) and N,N-dimethylformamide (2×250 mL). Fmoc groups were removed by treatment with 20% piperidine in N,N-dimethylformamide (1×5 min, 1×20 min, 2×220 mL). Resin was washed with N,N-dimethylformamide (2×250 mL), 2-propanol (2×250 mL), dichloromethane (2×250 mL) and N,N-dimethylformamide (2×250 mL). Solution of 3,5-bis(((tert-butoxycarbonyl)amino)methyl)benzoic acid (6, 24.2 g, 63.7 mmol), 5-chloro-1-((dimethylamino)(dimethyliminio)methyl)-1H-benzo[d][1,2,3]triazole 3-oxide tetrafluoroborate (TCTU, 22.6 g, 63.7 mmol) and N,N-diisopropylethylamine (20.0 mL, 115 mmol) in N,N-dimethylformamide (220 mL) was added to resin and mixture was shaken for 2.5 hours. Resin was washed with N,N-dimethylformamide (2×250 mL) and dichloromethane (10×250 mL). The product was cleaved from resin by treatment with 2,2,2-trifluoroethanol (400 mL) overnight. Resin was filtered off and washed with dichloromethane (2×200 mL). Solvents were evaporated and the residue was purified by flash column chromatography (Silicagel 60, 0.063-0.200 mm; eluent:dichloromethane/methanol 90:10) to give (S)-3-(2,6-bis(3,5-bis(((tert-butoxycarbonyl)amino)methyl)benzamido)hexanamido)propanoic acid (8) as white foam. Yield: 16.3 g (82%). RF (SiO2, dichloromethane/methanol 90:10): 0.30.

¹H NMR spectrum (300 MHz, CDCl₃, δH): 7.75-7.35 (m, 6H); 7.26-7.19 (m, 2H); 7.13 (bs, 1H); 5.61-5.35 (m, 4H); 4.76-4.61 (m, 1H); 4.25-4.08 (m, 8H); 3.60-3.26 (m, 4H); 2.60-2.45 (m, 2H); 2.02-1.85 (m, 1H); 1.85-1.69 (m, 1H); 1.62-1.51 (m, 2H); 1.46-1.39 (m, 38H). LC-MS: 942.1 (M+H)+.

The above compound (8, 16.1 g, 17.3 mmol) was dissolved in trifluoroacetic acid (80 mL) and left to stay for 30 minutes. The solvent was concentrated to ⅓ of volume and diethyl ether/cyclohexane mixture (1:1, 300 mL) was added. The resulting mixture was stirred overnight. The precipitate was collected by filtration, washed with diethyl ether and dried in vacuo affording (S)-((((6-((2-carboxyethyl)amino)-6-oxohexane-1,5-diyl)bis(azanediyl))bis(carbonyl))bis(benzene-5,1,3-triyl))tetramethanaminium 2,2,2-trifluoroacetate (9) as white powder. Yield: 16.5 g (96%). ¹H NMR spectrum (300 MHz, AcOD-d4, δH): 8.09 (dd, J=9.4 and 1.5 Hz, 4H); 7.84 (d, J=10.5 Hz, 2H); 4.76 (dd, J=8.2 and 6.1 Hz, 1H); 4.36 (s, 4H); 4.35 (s, 4H); 3.60-3.43 (m, 4H); 2.64 (t, J=6.5 Hz, 2H); 2.00-1.80 (m, 2H); 1.77-1.65 (m, 2H); 1.57-1.48 (m, 2H). LC-MS: 541.6 (M+H)+.

A suspension of 4-carboxy-3-fluorophenylboronic acid (10, 30.0 g, 163 mmol) and pinacol (21.2 g, 179 mmol) in toluene/ethanol mixture (1:1, 480 mL) was refluxed for 24 hours. Then the solvents were evaporated and co-evaporated with dichloromethane three times affording 2-fluoro-4-(4,4,5,5-tetramethyl-1,3,2-dioxaborolan-2-yl)benzoic acid (11) as white powder.

Yield: 43.3 g (100%). ¹H NMR spectrum (300 MHz, DMSO-d6, δH): 7.60 (t, J=7.3 Hz, 1H); 7.39 (d, J=7.5 Hz, 1H); 7.24 (d, J=10.6 Hz, 1H); 1.29 (s, 12H).

The acid (11, 35.2 g, 132 mmol) was dissolved in tetrahydrofuran (1:1, 600 mL), then 1-hydroxy-pyrrolidine-2,5-dione (HOSu, 25.2 g, 219 mmol) and N-(3-dimethylaminopropyl)-N′-ethylcarbodiimide hydrochloride (EDC.HCl, 42.0 g, 219 mmol) were added. The resulting mixture was stirred overnight at room temperature. Then the solvent was evaporated. The residue was dissolved in ethyl acetate (400 mL) and washed with water (2×300 mL) and brine (1×300 mL). Organic layer was dried over anhydrous sodium sulfate, filtered and evaporated. The product precipitated from ethyl acetate/cyclohexane mixture (1:4, 600 mL).

The precipitate was collected by filtration, washed with cyclohexane and dried in vacuo to yield 2,5-dioxopyrrolidin-1-yl 2-fluoro-4-(4,4,5,5-tetramethyl-1,3,2-dioxaborolan-2-yl)benzoate (12) as white powder. Yield: 45.2 g (94%). ¹H NMR spectrum (300 MHz, DMSO-d6, δH): 8.07 (t, J=7.3 Hz, 1H); 7.71 (d, J=7.7 Hz, 1H); 7.60 (d, J=10.8 Hz, 1H); 2.90 (s, 4H); 1.32 (s, 12H).

(S)-((((6-((2-Carboxyethyl)amino)-6-oxohexane-1,5-diyl)bis(azanediyl))bis(carbonyl))bis(benzene-5,1,3-triyl))tetramethanaminium 2,2,2-trifluoroacetate (9, 3.91 g, 3.92 mmol) was dissolved in water/N,N-dimethylformamide mixture (1:1, 80 mL). Subsequently N,N-diisopropylethylamine (6.15 mL, 35.3 mmol) and activated ester (12, 5.69 g, 15.7 mmol) were added. The mixture was stirred overnight at room temperature; then it was acidified by 1 M aqueous solution of hydrochloric acid. The solvent was co-evaporated with toluene three times. The residue was dissolved in ethyl acetate (150 mL) and washed with water (2×100 mL) and brine (1×100 mL). Organic layer was dried over anhydrous sodium sulfate, filtered and evaporated. The residue was treated with pinacol (0.06 g, 0.49 mmol) in tetrahydrofuran (70 mL) and evaporated from tetrahydrofuran three times. The residue was dried in vacuo affording the title compound (13) as beige solid. Yield: 5.82 g (95%). ¹H NMR spectrum (300 MHz, DMSO-d6, δH): 9.01-8.83 (m, 4H); 8.51-8.42 (m, 1H); 8.39-8.30 (m, 1H); 8.10-8.00 (m, 1H); 7.80-7.31 (m, 18H); 4.59-4.33 (m, 9H); 3.30-3.19 (m, 4H); 2.39 (t, J=6.7 Hz, 2H); 1.80-1.67 (m, 2H); 1.59-1.49 (m, 2H); 1.41-1.21 (m, 50H). LC-MS: 566.6 ((M−4×pinacol-4×H2O)/2+H)+.

### Example 13: 2,5-Dioxopyrrolidin-1-yl 3,5-bis((2-fluoro-4-(4,4,5,5-tetramethyl-1,3,2-dioxaborolan-2-yl)benzamido)methyl)benzoate

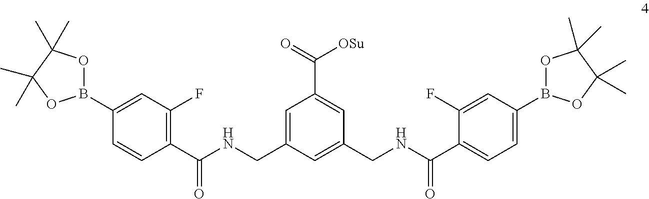

3,5-Bis(aminomethyl)benzoic acid dihydrochloride (2, 1.88 g, 7.43 mmol) was dissolved in water (20 mL). Subsequently N,N-diisopropylethylamine (10.4 mL, 59.5 mmol), N,N-dimethylformamide (40 mL) and 2,5-dioxopyrrolidin-1-yl 3-fluoro-4-(4,4,5,5-tetramethyl-1,3,2-dioxaborolan-2-yl)benzoate (1, 5.40 g, 14.8 mmol) were added.

The mixture was stirred overnight at room temperature; then it was acidified by 1 M aqueous solution of hydrochloric acid (200 mL). The solvent was co-evaporated with toluene three times. The residue was dissolved in dichloromethane/toluene mixture (1:1, 100 mL) and treated with pinacol (1.24 g, 10.5 mmol). The mixture was evaporated three times from toluene. The residue was dissolved in ethyl acetate (150 mL) and washed with water (3×100 mL). Organic layer was dried over anhydrous sodium sulfate, filtered and evaporated.

The residue was dissolved in dichloromethane (10 mL) and product started to precipitate. Then cyclohexane was added (190 mL) and the precipitate was collected by filtration, washed with cyclohexane and dried in vacuo to give 3,5-bis((3-fluoro-4-(4,4,5,5-tetramethyl-1,3,2-dioxaborolan-2-yl)benzamido)methyl)benzoic acid (3) as white powder.

Yield: 4.38 g (87%). ¹H NMR spectrum (300 MHz, DMSO-d6, δH): 12.95 (bs, 1H); 9.05-8.97 (m, 2H); 7.82 (s, 2H); 7.64 (t, J=7.3 Hz, 2H); 7.56-7.49 (m, 3H); 7.44-7.37 (m, 2H); 4.55-4.47 (m, 4H); 1.31 (s, 24H). LC-MS: 677.5 (M+H)+, 595.3 (M+H−pinacol)+, 513.3 (M+H−2×pinacol)+.

The above acid (3, 4.37 g, 6.48 mmol) was dissolved in acetonitrile/N,N-dimethylformamide mixture (4:1, 100 mL) and N-hydroxysuccinimide (HOSu, 0.89 g, 7.77 mmol) was added. The mixture was cooled down to 0° C. followed by addition of N,N-dicyclohexylcarbodiimide (DCC, 1.60 g, 7.77 mmol). The mixture was stirred for 30 minutes at 0° C. and overnight at room temperature. The insoluble by-product was filtered off and the filtrate was evaporated. The residue was dissolved in ethyl acetate (250 mL) and washed with water (2×150 mL). Organic layer was dried over anhydrous sodium sulfate, filtered and evaporated. The residue was dissolved in dichloromethane (10 mL) and cyclohexane was added (170 mL). The precipitate was collected by filtration, washed with cyclohexane. White powder was dissolved in tetrahydrofuran (100 mL). Pinacol (0.19 g, 1.60 mmol) and magnesium sulfate (10 g) were added to the solution and resulting mixture was stirred overnight at room temperature. The suspension was filtered through celite pad and the filtrate was evaporated. The residue was dissolved in dichloromethane (10 mL) and to the solution cyclohexane was added (170 mL). The precipitate was collected by filtration, washed with cyclohexane and dried in vacuo to give the title compound (4) as white powder. Yield: 3.99 g (80%). ¹H NMR spectrum (300 MHz, DMSO-d6, δH): 9.07 (t, J=5.7 Hz, 2H); 7.96 (s, 2H); 7.75 (s, 1H); 7.65 (t, J=7.2 Hz, 2H); 7.53 (d, J=7.7 Hz, 2H); 7.41 (d, J=10.4 Hz, 2H); 4.61-4.48 (m, 4H); 2.89 (s, 4H); 1.31 (s, 24H). LC-MS: 774.6 (M+H)+, 692.4 (M+H−pinacol)+, 610.3 (M+H−2×pinacol)+.

### Example 14

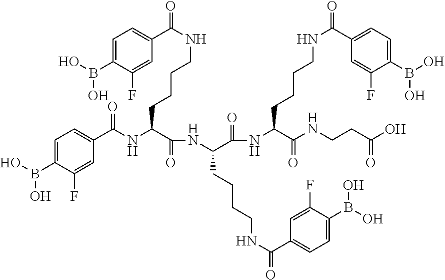 [SMILES: O=C(O)CCNC(=O)[C@H](CCCCNC(=O)c1ccc(B(O)O)c(F)c1)NC(=O)[C@H](CCCCNC(=O)c1ccc(B(O)O)c(F)c1)NC(=O)[C@H](CCCCNC(=O)c1ccc(B(O)O)c(F)c1)NC(=O)c1ccc(B(O)O)c(F)c1]

Prepared by solid-phase peptide synthesis from beta-Ala, Fmoc-Lys and pinacol 4-carboxy-2-fluorophenylboronate

### Example 15: 2,5-Dioxopyrrolidin-1-yl (R)-3-(2,4-bis(3-fluoro-4-(4,4,5,5-tetramethyl-1,3,2-dioxaborolan-2-yl)benzamido)butanamido)propanoate

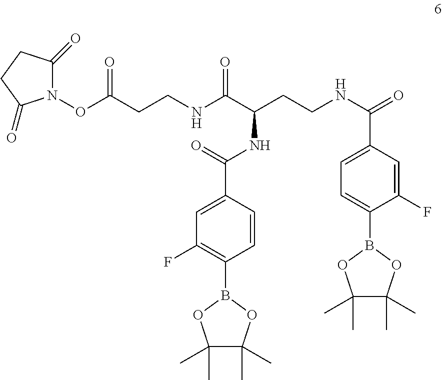 [SMILES: CC1(C)OB(c2ccc(C(=O)NCC[C@@H](NC(=O)c3ccc(B4OC(C)(C)C(C)(C)O4)c(F)c3)C(=O)NCCC(=O)ON3C(=O)CCC3=O)cc2F)OC1(C)C]

L-2,4-Diaminobutyric acid dihydrochloride (1, 4.81 g, 25.2 mmol) was suspended in a solution of sodium bicarbonate (10.6 g, 126 mmol) in water (80 mL). The mixture was heated until clear solution was formed. After cooling down to room temperature 1,4-dioxane (80 mL) and N-(9-fluorenylmethoxycarbonyloxy)succinimide (20.4 g, 60.4 mmol) were added. The mixture was stirred overnight at room temperature and then acidified with 5 M aqueous solution of hydrochloric acid. 1,4-Dioxane was evaporated, the aqueous phase was extracted with ethyl acetate (2×100 mL). Combined organic layers were washed with water (3×100 mL), dried over anhydrous sodium sulfate, filtered and evaporated. The residue was recrystallized from hot ethyl acetate/cyclohexane mixture twice. Product was collected by filtration, washed with cyclohexane and dried in vacuo to yield (R)-2,4-bis((((9H-fluoren-9-yl)methoxy)carbonyl)amino)butanoic acid (2) as white powder. Yield: 13.3 g (94%).

¹H NMR spectrum (300 MHz, DMSO-d6, δH): 12.62 (bs, 1H); 7.94-7.83 (m, 4H); 7.79-7.55 (m, 5H); 7.46-7.26 (m, 9H); 4.34-4.13 (m, 6H); 4.08-3.94 (m, 1H); 3.14-3.02 (m, 2H); 1.98-1.84 (m, 1H); 1.84-1.65 (m, 1H). LC-MS: 562.6 (M+H)+.

2-Chlorotrityl chloride resin 100-200 mesh 1.5 mmol/g (3, 5.84 g, 8.75 mmol) was left to swell in dry dichloromethane (70 mL) for 20 minutes. A solution of 3-((((9H-fluoren-9-yl)methoxy)carbonyl)amino)propanoic acid (Fmoc-Ala-OH, 1.82 g, 5.84 mmol) and N,N-diisopropylethylamine (3.86 mL, 22.2 mmol) in dry dichloromethane (50 mL) was added to resin and the mixture was shaken overnight. Resin was filtered and treated with a solution of N,N-diisopropylethylamine (2.03 mL, 11.7 mmol) in methanol/dichloromethane mixture (1:4, 1×10 min, 1×50 mL). Then resin was washed with dichloromethane (2×50 mL) and N,N-dimethylformamide (2×50 mL). Fmoc group was removed by treatment with 20% piperidine in N,N-dimethylformamide (1×5 min, 1×20 min, 2×50 mL). Resin was washed with N,N-dimethylformamide (2×50 mL), 2-propanol (2×50 mL), dichloromethane (2×50 mL) and N,N-dimethylformamide (2×50 mL). Solution of (R)-2,4-bis((((9H-fluoren-9-yl)methoxy)carbonyl)amino)butanoic acid (2, 6.57 g, 11.7 mmol), ethyl cyano-glyoxylate-2-oxime (Oxyma, 1.66 g, 11.7 mmol), N,N-diisopropylcarbodiimide (DIC, 1.81 mL, 11.7 mmol) and 2,4,6-collidine (3.09 mL, 23.4 mmol) in N,N-dimethylformamide (50 mL) was added to resin and mixture was shaken for 2.5 hours. Resin was filtered and washed with N,N-dimethylformamide (2×50 mL), dichloromethane (2×50 mL) and N,N-dimethylformamide (2×50 mL). Fmoc groups were removed by treatment with 20% piperidine in N,N-dimethylformamide (1×5 min, 1×20 min, 2×50 mL). Resin was washed with N,N-dimethylformamide (2×50 mL), 2-propanol (2×50 mL), dichloromethane (2×50 mL) and N,N-dimethylformamide (2×50 mL). Solution of 2,5-dioxopyrrolidin-1-yl 3-fluoro-4-(4,4,5,5-tetramethyl-1,3,2-dioxaborolan-2-yl)benzoate (4, 8.48 g, 23.4 mmol) and N,N-diisopropylethylamine (7.32 mL, 42.0 mmol) in N,N-dimethylformamide (50 mL) was added to resin and mixture was shaken for 2 hours. Resin was filtered and washed with N,N-dimethylformamide (3×60 mL) and dichloromethane (10×60 mL). The product was cleaved from resin by treatment with 1,1,1,3,3,3-hexafluoro-2-propanol/dichloromethane mixture (1:2, 90 mL) for 2 hours. Resin was filtered off and washed with dichloromethane (4×50 mL). Solvents were evaporated; the residue was dissolved in ethyl acetate (100 mL) and washed with water (2×80 mL) and brine (1×80 mL). Organic layer was dried over anhydrous sodium sulfate, filtered and evaporated to dryness affording (R)-3-(2,4-bis(3-fluoro-4-(4,4,5,5-tetramethyl-1,3,2-dioxaborolan-2-yl)benzamido)butanamido)propanoic acid (5) as beige solid. Yield: 3.25 g (81%). ¹H NMR spectrum (300 MHz, CDCl₃, δH): 12.11 (bs, 1H); 8.69 (d, J=7.9 Hz, 1H); 8.63-8.52 (m, 1H); 8.14-8.03 (m, 1H); 7.81-7.61 (m, 5H); 7.56 (d, J=10.5 Hz, 1H); 4.54-4.39 (m, 1H); 3.44-3.17 (m, 4H); 2.43-2.33 (m, 2H); 2.14-1.99 (m, 1H); 1.99-1.85 (m, 1H); 1.31 (s, 24H). LC-MS: 521.0 (M−2×pinacol+H)+, 603.1 (M−pinacol+H)+, 685.3 (M+H)+.

The acid (5, 3.24 g, 4.73 mmol) was dissolved in dichloromethane (50 mL) followed by addition of N-hydroxysuccinimide (0.65 g, 5.67 mmol) and N-(3-dimethylaminopropyl)-N-ethylcarbodiimide hydrochloride (1.09 g, 5.67 mmol). The mixture was stirred overnight then it was diluted with dichloromethane (50 mL) and washed with water (2×80 mL) and brine (1×80 mL). Organic layer was dried over anhydrous sodium sulfate, filtered and evaporated to dryness affording the title compound (6) as white solid. Yield: 3.42 g (92%). ¹H NMR spectrum (300 MHz, DMSO-d6, δH): 8.72 (d, J=7.9 Hz, 1H); 8.57 (t, J=5.4 Hz, 1H); 8.22 (t, J=5.5 Hz, 1H); 7.77-7.62 (m, 5H); 7.56 (d, J=10.1 Hz, 1H); 4.53-4.40 (m, 1H); 3.48-3.27 (m, 4H); 2.86 (t, J=7.1 Hz, 2H); 2.80 (s, 4H); 2.15-2.02 (m, 1H); 2.02-1.88 (m, 1H); 1.31 (s, 24H). LC-MS: 618.1 (M−2×pinacol+H)+, 700.2 (M−pinacol+H)+, 782.4 (M+H)+.

### Example 16: 3-(3-Fluoro-5-(4,4,5,5-tetramethyl-1,3,2-dioxaborolan-2-yl)benzoyl)-5-(4,4,5,5-tetramethyl-1,3,2-dioxaborolan-2-yl)benzoic acid

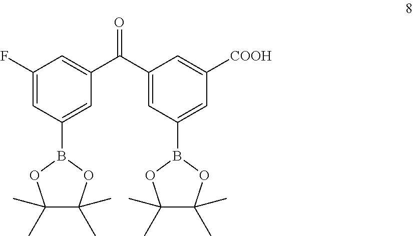 [SMILES: CC1(C)OB(c2cc(F)cc(C(=O)c3cc(B4OC(C)(C)C(C)(C)O4)cc(C(=O)O)c3)c2)OC1(C)C]

3-Bromo-5-iodobenzoic acid (1, 16.4 g, 50.0 mmol) was suspended in methanol (100 mL) and methanesulfonic acid (1 mL) was added. The resulting mixture was stirred for 16 hours at 60° C. (oil bath). The resulting clear solution was cooled to −20° C. in the freezer for 16 hours and the resulting solid was collected by filtration, washed with chilled (−20° C.) methanol and dried in vacuo to give methyl 3-bromo-5-iodobenzoate (2) as an off-white solid.

Yield: 13.9 g (82%). ¹H NMR spectrum (300 MHz, CDCl₃, δH): 8.30 (s, 1H); 8.14 (s, 1H); 8.04 (s, 1H); 3.93 (s, 1H).

1,3-Dibromo-5-fluorobenzene (3, 6.30 mL, 50.0 mmol) was dissolved in dry diethyl ether (150 mL) and cooled down to −78 C. 2.35 M n-Butyllithium in hexane (22.0 mL, 52.5 mmol) was added dropwise with stirring. After 15 minutes, dry N,N-dimethylformamide (7.70 mL, 100 mmol) was added and the resulting mixture was stirred at for 15 minutes and then allowed to warm to ambient temperature. After one hour, the reaction mixture was quenched with 1 M aqueous solution of hydrochloric acid (150 mL). Layers were separated and the organic layer was washed with brine (100 mL), dried over anhydrous magnesium sulfate and evaporated to give 3-bromo-5-fluorobenzaldehyde (4) as yellowish oil which solidified on storage in freezer. Yield: 10.2 g (100%). ¹H NMR spectrum (300 MHz, CDCl₃, δH): 9.92 (s, 1H); 7.80 (bs, 1H); 7.50 (bs, 2H).

Methyl 3-bromo-5-iodobenzoate (2, 6.80 g, 20.0 mmol) was dissolved in dry tetrahydrofuran (50 mL) under nitrogen atmosphere and cooled down to −40° C. 1.3 M Isopropylmagnesium chloride-lithium chloride complex in tetrahydrofuran (16.1 mL, 21.0 mmol) was added dropwise via an addition funnel. After 30 minutes 3-bromo-5-fluorobenzaldehyde (4) (4.87 g, 24.0 mmol) was added with the aid of dry tetrahydrofuran (5 mL). The resulting mixture was allowed to warm to room temperature over an hour and stirred for one more hour at ambient temperature. The reaction was quenched by addition of 0.5 M aqueous solution of hydrochloric acid (50 mL) and extracted with diethyl ether (1×200 mL). Organic layer was washed with brine (100 mL) and dried over anhydrous sodium sulfate, filtered and evaporated. The residue 5 was dissolved in dry dichloromethane (100 mL) and pyridinium chlorochromate (PCC, 6.45 g, 30.0 mmol) was added. The reaction mixture was then stirred overnight (16 hours) before it was quenched with 2-propanol (3 mL). After stirring for one hour at room temperature, the reaction mixture was filtered through a silica gel plug (100 g) topped with celite S and washed with dichloromethane (2×100 mL). The solvent was removed in vacuo and the residue was purified by flash column chromatography (Silicagel 60, 0.063-0.200 mm; eluent:cyclohexane/dichloromethane 6:1 to 2:1) to give methyl 3-bromo-5-(3-bromo-5-fluorobenzoyl)benzoate (6) as colorless solid.

Yield: 7.10 g (85%). ¹H NMR spectrum (300 MHz, CDCl₃, δH): 8.43 (s, 1H); 8.30 (s, 1H); 8.11 (s, 1H); 7.71 (s, 1H); 7.53 (d, J=7.6 Hz, 1H); 7.41 (d, J=8.3 Hz, 1H); 3.97 (s, 3H).

LC-MS: neither molecular oil nor fragments could be detected.

A 250 mL reaction vessel was charged with potassium acetate (6.70 g, 68.4 mmol) and the salt was dried for 1 hour at 110° C. in vacuo. After cooling to room temperature, the reaction vessel was backfilled with nitrogen and charged with methyl 3-bromo-5-(3-bromo-5-fluorobenzoyl)benzoate (6, 7.10 g, 481 mol), palladium acetate (77.0 mg, 342 mol), 2-dicyclohexylphosphino-2,4,6-triisopropylbiphenyl (XPhos, 325 mg, 684 mol) and bis(pinacolato)diboron (9.53 mg, 37.6 mmol). The reaction vessel was then evacuated and backfilled with nitrogen (this procedure was repeated twice), anhydrous tetrahydrofuran (3 mL) was added with syringe, the vessel was sealed with a plastic stopper and submerged in the heating bath preheated to 60 C. After stirring at 400 rpm for 16 hours (overnight) the reaction mixture was cooled to ambient temperature, diluted with dichloromethane (100 mL) and filtered through a short plug of silica (70 g) topped with celite S with the aid of dichloromethane (3×70 mL). The filtrate was concentrated under reduced pressure to afford the product as yellowish waxy foam, which was triturated with ice-cold n-hexane (70 mL) to cause crystallization. The resulting solid collected by filtration and dried in vacuo to give methyl 3-(3-fluoro-5-(4,4,5,5-tetramethyl-1,3,2-dioxaborolan-2-yl)benzoyl)-5-(4,4,5,5-tetramethyl-1,3,2-dioxaborolan-2-yl)benzoate (7) as white solid. Yield: 7.10 g (88%).

¹H NMR spectrum (300 MHz, CDCl₃, δH): 8.69 (s, 1H); 8.48 (s, 1H); 8.38 (s, 1H); 7.97 (s, 1H); 7.72 (d, J=8.5 Hz, 1H); 7.54 (d, J=9.0 Hz, 1H); 3.95 (s, 3H); 1.36 (s, 12H); 1.35 (s, 12H). LC-MS: 511.6 (M+H)+.

Methyl 3-(3-fluoro-5-(4,4,5,5-tetramethyl-1,3,2-dioxaborolan-2-yl)benzoyl)-5-(4,4,5,5-tetramethyl-1,3,2-dioxaborolan-2-yl)benzoate (7, 7.10 g, 13.9 mmol) was suspended in methanol (42 mL) and water (13 mL). Lithium hydroxide (2.91 g, 69.5 mmol) was added and the resulting mixture was vigorously stirred at ambient temperature for 16 hours. The reaction mixture was diluted with water (120 mL) and extracted with diethyl ether (70 mL). The ethereal layer was discarded and the aqueous layer acidified with concentrated hydrochloric acid (10 mL) and extracted with ethyl acetate (100 mL). The organic layer was washed with brine (100 mL) dried over anhydrous sodium sulfate, filtered and evaporated. The crude product was dissolved in hot ethyl acetate (80 mL) and pinacol was added until clear solution was obtained. The solution was evaporated to dryness and then evaporated twice from dichloromethane (2×40 mL) to give the title 3-(3-fluoro-5-(4,4,5,5-tetramethyl-1,3,2-dioxaborolan-2-yl)benzoyl)-5-(4,4,5,5-tetramethyl-1,3,2-dioxaborolan-2-yl)benzoic acid (8) as colorless solid. The compound contains residual pinacol which could not be removed. Yield: 6.82 g (99%). ¹H NMR spectrum (300 MHz, CDCl₃, δH): 8.77 (s, 1H); 8.54 (t, J=1.8 Hz, 1H); 8.44 (d, J=1.1 Hz, 1H); 7.98 (s, 1H); 7.80-7.68 (m, 1H); 7.63-7.50 (m, 1H); 1.37 (s, 12H); 1.35 (s, 12H). LC-MS: 497.5 (M+H)+, 415.4 (M−pinacol+H)+.

### Example 17: (2,5-dioxopyrrolidin-1-yl) 3,5-bis[[[4-(4,4,5,5-tetramethyl-1,3,2-dioxaborolan-2-yl)benzoyl]amino]methyl]benzoate

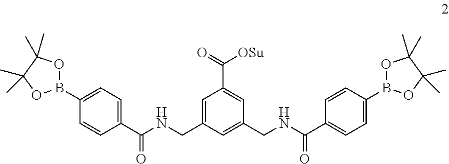

3,5-Bis((4-(4,4,5,5-tetramethyl-1,3,2-dioxaborolan-2-yl)benzamido)methyl)benzoic acid (1, 2.57 g, 4.00 mmol) was dissolved in acetonitrile/N,N-dimethylformamide mixture (3:1, 100 mL). N-hydroxysuccinimide (0.55 g, 4.80 mmol) was added. The mixture was cooled down to 0° C. followed by addition of N,N-dicyclohexylcarbodiimide (0.99 g, 4.80 mmol). The mixture was stirred for 30 minutes at 0° C. and overnight at room temperature. The insoluble by-product was filtered off and the filtrate was evaporated. The residue was dissolved in ethyl acetate (250 mL) and washed with water (2×150 mL). Organic layer was dried over anhydrous sodium sulfate, filtered and evaporated. The residue was dissolved in toluene (10 mL) and product started to precipitate. Cyclohexane was added (170 mL). The precipitate was collected by filtration, washed with cyclohexane and dried in vacuo to yield the title compound (2) as white powder. The product contains traces of N,N-dicyclohexylurea.

Yield: 2.85 g (97%). ¹H NMR spectrum (300 MHz, DMSO-d6, δH): 9.24 (t, J=5.7 Hz, 2H); 7.95-7.83 (m, 6H); 7.79-7.70 (m, 5H); 4.60-4.52 (m, 4H); 2.87 (s, 4H); 1.31 (s, 24H). LC-MS: 737.4 (M+H)+, 655.2 (M+H−pinacol)+, 573.1 (M+H−2×pinacol

### Example 18: N2,N6-Bis(6-fluoro-1-hydroxy-1,3-dihydrobenzo[c][1,2]oxaborole-5-carbonyl)-L-lysine

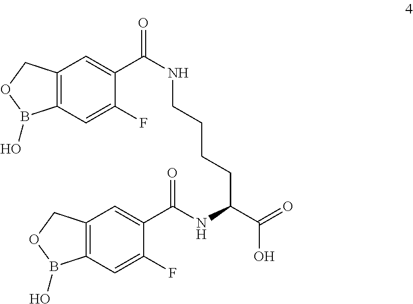 [SMILES: O=C(NCCCC[C@H](NC(=O)c1cc2c(cc1F)B(O)OC2)C(=O)O)c1cc2c(cc1F)B(O)OC2]

6-Fluoro-1-hydroxy-1,3-dihydrobenzo[c][1,2]oxaborole-5-carboxylic acid (1, 6.00 g, 30.6 mmol)) was dissolved in tetrahydrofuran (80 mL). N,N-Dimethylformamide (10 mL), N-hydroxysuccinimide (3.87 g, 33.7 mmol) and N-(3-dimethylaminopropyl)-N-ethylcarbodiimide hydrochloride (6.46 g, 33.7 mmol) were added at room temperature. After stirring for 2 hours, volatiles were evaporated under reduced pressure and the residue was redissolved in ethyl acetate (200 mL) and washed with 1 M aqueous hydrochloric acid (2×60 mL). Organic portion was dried using anhydrous sodium sulfate. Volatiles were evaporated under reduced pressure to give 2,5-dioxopyrrolidin-1-yl 6-fluoro-1-hydroxy-1,3-dihydrobenzo[c][1,2]oxaborole-5-carboxylate (2) as a white solid. Yield: 8.35 g (93%).

¹H NMR spectrum (300 MHz, DMSO-d6, δH): 9.65 (s, 1H); 8.11 (d, J=5.9 Hz, 1H); 7.71 (d, J=10.1 Hz, 1H); 5.08 (s, 2H); 2.90 (s, 4H). LC-MS: 294.4 (M+H)+.

L-Lysine hydrochloride (3, 1.56 g, 8.50 mmol) was dissolved in N,N-dimethylformamide (50 mL) and water (25 mL). N,N-Diisopropylethylamine (8.92 mL, 51.2 mmol) and 2,5-dioxopyrrolidin-1-yl 6-fluoro-1-hydroxy-1,3-dihydrobenzo[c][1,2]oxaborole-5-carboxylate (2, 5.00 g, 17.0 mmol) were added at room temperature. After stirring for 3 hours, volatiles were evaporated under reduced pressure and the residue was precipitated by aqueous 1 M hydrochloric acid. Precipitate was washed by water and purified by precipitation from acetonitrile/water mixture, collected by centrifuge and freeze-dried to afford N2,N6-bis(6-fluoro-1-hydroxy-1,3-dihydrobenzo[c][1,2]oxaborole-5-carbonyl)-L-lysine (4) as a white solid.

Yield: 3.25 g (76%). ¹H NMR spectrum (300 MHz, DMSO-d6, δH): 12.66 (bs, 1H); 9.41 (d, J=5.7 Hz, 2H); 8.59 (d, J=7.2 Hz, 1H); 8.39 (t, J=5.0 Hz, 1H); 7.61-7.53 (m, 2H); 7.53-7.44 (m, 2H); 4.97 (d, J=5.7 Hz, 4H); 4.41-4.30 (m, 1H); 3.30-3.20 (m, 2H); 1.90-1.70 (m, 2H); 1.59-1.38 (m, 4H). LC-MS: 503.5 (M+H)+.

### Example 19: (S)-2,3-Bis(4-fluoro-1-hydroxy-1,3-dihydrobenzo[c][1,2]oxaborole-6-carboxamido)propanoic acid

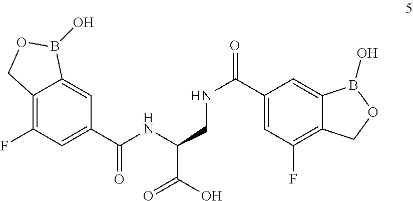 [SMILES: O=C(NC[C@H](NC(=O)c1cc(F)c2c(c1)B(O)OC2)C(=O)O)c1cc(F)c2c(c1)B(O)OC2]

Solution of methyl 4-(bromomethyl)-3-fluoro-5-(4,4,5,5-tetramethyl-1,3,2-dioxaborolan-2-yl)benzoate (1, 23.0 g, 61.7 mmol) and sodium hydroxide (12.3 g, 0.31 mol) in water (400 mL) was stirred overnight at ambient temperature. 6 M Aqueous solution of hydrochloric acid (60 mL, 6 M) was added to the reaction mixture resulting to white precipitate. The flask with the precipitate was kept in fridge for 1 hour. Then it was filtered and the filtration cake was washed with water (200 mL) and freeze-dried to afford 4-fluoro-1-hydroxy-1,3-dihydrobenzo[c][1,2]oxaborole-6-carboxylic acid (2) as white solid.

Yield: 12.1 g (100%). ¹H NMR spectrum (300 MHz, DMSO-d6, δH): 13.24 (bs, 1H), 9.58 (s, 1H), 8.20 (s, 1H), 7.73 (d, J=9.9 Hz, 1H), 5.14 (s, 2H).

4-Fluoro-1-hydroxy-1,3-dihydrobenzo[c][1,2]oxaborole-6-carboxylic acid (2, 4.00 g, 20.4 mmol), N-hydroxysuccinimide (2.35 g, 20.4 mmol) and 1-ethyl-3-(3′-dimethylaminopropyl) carbodiimide hydrochloride (3.91 g, 20.4 mmol) were stirred in tetrahydrofuran (120 mL) and N,N-dimethylformamide (20 mL) for 3.5 hours at ambient temperature. The reaction mixture was evaporated and extracted with ethyl acetate (3×150 mL) and 1 M aqueous solution of hydrochloric acid (150 mL). The organic phase was dried over anhydrous sodium sulfate, filtered and evaporated to afford 2,5-dioxopyrrolidin-1-yl 4-fluoro-1-hydroxy-1,3-dihydrobenzo[c][1,2]oxaborole-6-carboxylate (3) as white solid. Yield: 5.68 g (97%). LC-MS: 294.3 (M+H)+.

Solution of 2,5-dioxopyrrolidin-1-yl 4-fluoro-1-hydroxy-1,3-dihydrobenzo[c][1,2]oxaborole-6-carboxylate (3, 5.10 g, 17.4 mmol), (S)-2,3-diaminopropanoic acid hydrochloride (4, 1.22 g, 8.70 mmol) and N,N-diisopropylethylamine (9.28 mL, 52.2 mmol) in N,N-dimethylformamide (100 mL) and water (10 mL) was stirred at ambient temperature overnight. The reaction mixture was evaporated and extracted with ethyl acetate (2×250 mL) and 1 M aqueous solution of hydrochloric acid (150 mL), organic layers were washed with brine (200 mL). The organic phase was dried over anhydrous sodium sulfate, filtered and evaporated to afford (S)-2,3-bis(4-fluoro-1-hydroxy-1,3-dihydrobenzo[c][1,2]oxaborole-6-carboxamido)propanoic acid (5) as white solid. Yield: 3.53 g (88%). ¹H NMR spectrum (300 MHz, DMSO-d6, δH): 12.82 (bs, 1H); 9.54 (d, J=6.2 Hz, 2H); 8.86 (d, J=7.7 Hz, 1H); 8.78 (t, J=6.0 Hz, 1H); 8.10 (s, 1H); 8.04 (s, 1H); 7.80-7.63 (m, 2H); 5.13 (d, J=6.2 Hz, 4H); 4.77-4.62 (m, 1H); 3.91-3.77 (m, 1H); 3.77-3.62 (m, 1H). LC-MS: 461.3 (M+H)+.

### Example 20: 2,5-Dioxopyrrolidin-1-yl 3-(2,4-difluoro-5-(4,4,5,5-tetramethyl-1,3,2-dioxaborolan-2-yl)benzoyl)-5-(4,4,5,5-tetramethyl-1,3,2-dioxaborolan-2-yl)benzoate

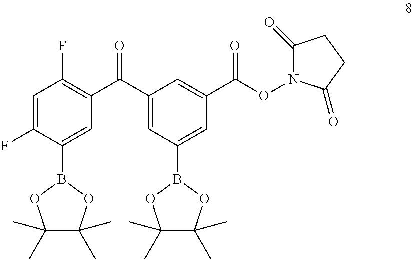 [SMILES: CC1(C)OB(c2cc(C(=O)ON3C(=O)CCC3=O)cc(C(=O)c3cc(B4OC(C)(C)C(C)(C)O4)c(F)cc3F)c2)OC1(C)C]

3-Bromo-5-iodobenzoic acid (1, 5.00 g, 15.3 mmol) was dissolved in anhydrous dichloromethane (100 mL) and tert-butanol (1.52 mL, 16.1 mmol), N,N′-dicyclohexylcarbodiimide (3.31 mL, 16.1 mmol) and 4-(dimethylamino)pyridine (1.96 mL, 16.1 mmol) were added. The reaction mixture was stirred at room temperature for 16 hours. Reaction mixture was then washed with 1 M aqueous solution of hydrochloric acid (2×50 mL) and brine (1×40 mL). Organic portion was dried over anhydrous sodium sulfate. Volatiles were evaporated under reduced pressure and the residue was purified by column chromatography (Silicagel 60, 0.063-0.200 mm; eluent:cyclohexane/ethyl acetate 10:1) to give tert-butyl 3-bromo-5-iodobenzoate (2) as a white solid. Yield: 4.67 g (80%). NMR spectrum (300 MHz, CDCl₃, δH): 8.22 (s, 1H); 8.06 (s, 1H); 8.00 (s, 1H); 1.58 (s, 9H).

tert-Butyl 3-bromo-5-iodobenzoate (2, 4.31 g, 11.3 mmol) was dissolved in anhydrous tetrahydrofuran (50 mL) under nitrogen atmosphere and cooled down to −40° C. 1.3 M Isopropylmagnesium chloride-lithium chloride complex in tetrahydrofuran (9.52 mL, 12.4 mmol) was added slowly dropwise. After 40 minutes 5-bromo-2,4-difluorobenzaldehyde (3, 2.86 g, 12.9 mmol) was added with the aid of dry tetrahydrofuran (5 mL). The resulting mixture was allowed to warm to room temperature overnight (16 hours). The reaction was quenched by addition of 0.5 M aqueous solution of hydrochloric acid (15 mL) and extracted with ethyl acetate (2×100 mL). Organic layer was washed with brine (40 mL) and dried over anhydrous sodium sulfate. Volatiles were evaporated under reduced pressure and the residue was purified by column chromatography (Silicagel 60, 0.063-0.200 mm; eluent: cyclohexane/ethyl acetate 10:1) to give tert-butyl 3-bromo-5-((5-bromo-2,4-difluorophenyl)(hydroxy)methyl)benzoate (4) as a white solid. Yield: 4.38 g (81%).

¹H NMR spectrum (300 MHz, CDCl₃, δH): 8.02 (t, J=1.6 Hz, 1H); 7.92 (s, 1H); 7.77-7.65 (m, 2H); 6.89 (dd, J=9.7 and 8.3, 1H); 6.09 (d, J=3.9 Hz, 1H); 2.43 (d, J=4.0 Hz, 1H); 1.68-1.58 (m, 9H).

tert-Butyl 3-bromo-5-((5-bromo-2,4-difluorophenyl)(hydroxy)methyl)benzoate (4) was dissolved in dry dichloromethane (50 mL) and pyridinium chlorochromate (PCC, 2.96 g, 13.7 mmol) was added. The reaction mixture was then stirred overnight (16 hours) before it was quenched with 2-propanol (1.5 mL). After stirring for one hour at room temperature, the reaction mixture was filtered through a short plug of celite (5 g) and washed with dichloromethane (50 mL). Volatiles were removed under reduced pressure and the residue was purified by flash column chromatography (Silicagel 60, 0.063-0.200 mm; eluent: cyclohexane/ethyl acetate 20:1) to give tert-butyl 3-bromo-5-(5-bromo-2,4-difluorobenzoyl)benzoate (5) as colorless solid. Yield: 4.20 g (96%). ¹H NMR spectrum (300 MHz, CDCl₃, δH): 8.33 (t, J=1.7 Hz, 1H); 8.28-8.24 (m, 1H); 8.10-8.07 (m, 1H); 7.86 (t, J=7.3 Hz, 1H); 7.03 (dd, J=9.3 and 8.1 Hz, 1H); 1.61 (s, 9H).

tert-Butyl 3-bromo-5-(5-bromo-2,4-difluorobenzoyl)benzoate (5, 4.20 g, 8.82 mmol), palladium acetate (59.0 mg, 0.26 mmol), 2-dicyclohexylphosphino-2,4,6-triisopropylbiphenyl (XPhos, 252 mg, 0.52 mmol), potassium acetate (3.46 g, 35.3 mmol) and bis(pinacolato)diboron (4.70 g, 18.5 mmol) were mixed in the reaction flask and the resulting mixture was evacuated and backfilled with argon (this procedure was repeated twice). Anhydrous tetrahydrofuran (60 mL) was added with syringe, the vessel was sealed with rubber septum and submerged in the heating bath preheated to 60 C. After stirring for 16 hours, the reaction mixture was cooled to ambient temperature, diluted with cyclohexane (100 mL) and filtered through a short plug of celite with the aid of dichloromethane (100 mL). Volatiles were removed under reduced pressure and the residue was purified by flash column chromatography (Silicagel 60, 0.063-0.200 mm; eluent:cyclohexane/ethyl acetate 10:1) to give tert-butyl 3-(2,4-difluoro-5-(4,4,5,5-tetramethyl-1,3,2-dioxaborolan-2-yl)benzoyl)-5-(4,4,5,5-tetramethyl-1,3,2-dioxaborolan-2-yl)benzoate (6) as yellow solid. Yield: 4.78 g (95%). ¹H NMR spectrum (300 MHz, CDCl₃, δH): 8.61 (s, 1H); 8.41 (d, J=1.5 Hz, 1H); 8.34 (s, 1H); 8.05 (dd, J=8.3 and 6.7 Hz, 1H); 6.96-6.81 (m, 1H); 1.61 (s, 9H); 1.36 (d, J=2.2 Hz, 24H).

tert-Butyl 3-(2,4-difluoro-5-(4,4,5,5-tetramethyl-1,3,2-dioxaborolan-2-yl)benzoyl)-5-(4,4,5,5-tetramethyl-1,3,2-dioxaborolan-2-yl)benzoate (6, 4.78 g, 8.38 mmol) was dissolved in dichloromethane (10 mL) and trifluoroacetic acid (40 mL) was added at room temperature. Reaction mixture was stirred for 3 hours. Volatiles were removed under reduced pressure and the residue was co-evaporated with dichloromethane (4×50 mL). Resulting 3-(2,4-difluoro-5-(4,4,5,5-tetramethyl-1,3,2-dioxaborolan-2-yl)benzoyl)-5-(4,4,5,5-tetramethyl-1,3,2-dioxaborolan-2-yl)benzoic acid (7) was used for the next step without further purification.

Yield: 4.10 g (96%). ¹H NMR spectrum (300 MHz, CDCl₃, δH): 8.75 (s, 1H); 8.50 (t, J=1.7 Hz, 1H); 8.46 (s, 1H); 8.09 (dd, J=8.4 and 6.8 Hz, 1H); 6.95-6.83 (m, 1H); 1.37 (d, J=1.8 Hz, 24H).

3-(2,4-Difluoro-5-(4,4,5,5-tetramethyl-1,3,2-dioxaborolan-2-yl)benzoyl)-5-(4,4,5,5-tetramethyl-1,3,2-dioxaborolan-2-yl)benzoic acid (7, 4.10 g, 8.00 mmol) was dissolved in dichloromethane (50 mL) and N-hydroxysuccinimide (1.29 g, 11.2 mmol) and N-(3-dimethylaminopropyl)-N-ethylcarbodiimide hydrochloride (2.14 g, 11.2 mmol) were added at room temperature. After stirring for 6 hours, the reaction mixture was washed with 10% aqueous solution of potassium bisulfate (2×100 mL) and brine (30 mL). Organic portion was dried using anhydrous sodium sulfate. Volatiles were evaporated under reduced pressure to give 2,5-dioxopyrrolidin-1-yl 3-(2,4-difluoro-5-(4,4,5,5-tetramethyl-1,3,2-dioxaborolan-2-yl)benzoyl)-5-(4,4,5,5-tetramethyl-1,3,2-dioxaborolan-2-yl)benzoate (8) as a yellow solid.

Yield: 4.84 g (99%). ¹H NMR spectrum (300 MHz, CDCl₃, δH): 8.81-8.75 (m, 1H); 8.57-8.51 (m, 1H); 8.47 (s, 1H); 8.08 (dd, J=8.5 and 6.7 Hz, 1H); 6.89 (dd, J=9.9 and 9.0 Hz, 1H); 2.92 (bs, 4H); 1.36 (s, 24H). LC-MS: 448.4 (M−2Pinacol+H)+.

### Example 21: 2,5-Dioxopyrrolidin-1-yl 3,5-bis((2-fluoro-4-(4,4,5,5-tetramethyl-1,3,2-dioxaborolan-2-yl)benzamido)methyl)benzoate

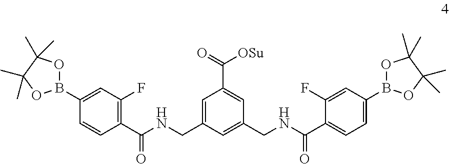

3,5-Bis(aminomethyl)benzoic acid dihydrochloride (2, 1.88 g, 7.43 mmol) was dissolved in water (20 mL). Subsequently N,N-diisopropylethylamine (10.4 mL, 59.5 mmol), N,N-dimethylformamide (40 mL) and 2,5-dioxopyrrolidin-1-yl 3-fluoro-4-(4,4,5,5-tetramethyl-1,3,2-dioxaborolan-2-yl)benzoate (1, 5.40 g, 14.8 mmol) were added.

The mixture was stirred overnight at room temperature; then it was acidified by 1 M aqueous solution of hydrochloric acid (200 mL). The solvent was co-evaporated with toluene three times. The residue was dissolved in dichloromethane/toluene mixture (1:1, 100 mL) and treated with pinacol (1.24 g, 10.5 mmol). The mixture was evaporated three times from toluene. The residue was dissolved in ethyl acetate (150 mL) and washed with water (3×100 mL). Organic layer was dried over anhydrous sodium sulfate, filtered and evaporated. The residue was dissolved in dichloromethane (10 mL) and product started to precipitate. Then cyclohexane was added (190 mL) and the precipitate was collected by filtration, washed with cyclohexane and dried in vacuo to give 3,5-bis((3-fluoro-4-(4,4,5,5-tetramethyl-1,3,2-dioxaborolan-2-yl)benzamido)methyl)benzoic acid (3) as white powder. Yield: 4.38 g (87%). ¹H NMR spectrum (300 MHz, DMSO-d6, δH): 12.95 (bs, 1H); 9.05-8.97 (m, 2H); 7.82 (s, 2H); 7.64 (t, J=7.3 Hz, 2H); 7.56-7.49 (m, 3H); 7.44-7.37 (m, 2H); 4.55-4.47 (m, 4H); 1.31 (s, 24H). LC-MS: 677.5 (M+H)+, 595.3 (M+H−pinacol)+, 513.3 (M+H−2×pinacol)+.

The above acid (3, 4.37 g, 6.48 mmol) was dissolved in acetonitrile/N,N-dimethylformamide mixture (4:1, 100 mL) and N-hydroxysuccinimide (HOSu, 0.89 g, 7.77 mmol) was added. The mixture was cooled down to 0° C. followed by addition of N,N-dicyclohexylcarbodiimide (DCC, 1.60 g, 7.77 mmol). The mixture was stirred for 30 minutes at 0° C. and overnight at room temperature. The insoluble by-product was filtered off and the filtrate was evaporated. The residue was dissolved in ethyl acetate (250 mL) and washed with water (2×150 mL). Organic layer was dried over anhydrous sodium sulfate, filtered and evaporated. The residue was dissolved in dichloromethane (10 mL) and cyclohexane was added (170 mL). The precipitate was collected by filtration, washed with cyclohexane. White powder was dissolved in tetrahydrofuran (100 mL). Pinacol (0.19 g, 1.60 mmol) and magnesium sulfate (10 g) were added to the solution and resulting mixture was stirred overnight at room temperature. The suspension was filtered through celite pad and the filtrate was evaporated. The residue was dissolved in dichloromethane (10 mL) and to the solution cyclohexane was added (170 mL). The precipitate was collected by filtration, washed with cyclohexane and dried in vacuo to give the title compound (4) as white powder. Yield: 3.99 g (80%). ¹H NMR spectrum (300 MHz, DMSO-d6, δH): 9.07 (t, J=5.7 Hz, 2H); 7.96 (s, 2H); 7.75 (s, 1H); 7.65 (t, J=7.2 Hz, 2H); 7.53 (d, J=7.7 Hz, 2H); 7.41 (d, J=10.4 Hz, 2H); 4.61-4.48 (m, 4H); 2.89 (s, 4H); 1.31 (s, 24H). LC-MS: 774.6 (M+H)+, 692.4 (M+H−pinacol)+, 610.3 (M+H−2×pinacol)+.

### Example 22: (3-(4,4,5,5-Tetramethyl-1,3,2-dioxaboroloan-2-yl)-5-((3-(4,4,5,5-tetramethyl-1,3,2-dioxaborolan-2-yl)-5-(trifluormethyl)phenyl)sulfonyl)benzoyl)glycine

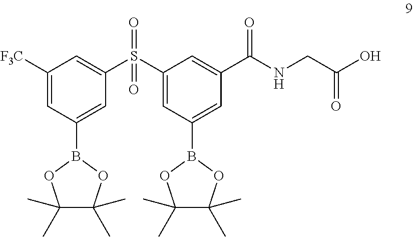 [SMILES: CC1(C)OB(c2cc(C(=O)NCC(=O)O)cc(S(=O)(=O)c3cc(B4OC(C)(C)C(C)(C)O4)cc(C(F)(F)F)c3)c2)OC1(C)C]

1,3-Dibromo-5-(trifluoromethyl)benzene (1, 13.1 g, 43.1 mmol) was added to a mixture of copper(II) sulfate pentahydrate (541 mg, 2.36 mmol) and potassium hydroxide (9.24 g, 216 mmol) in mixture dimethyl sulfoxide/water (10:1, 70 mL), reaction flask was filled with nitrogen and at the end was added 1,2-ethanedithiol (6.00 mL, 90.5 mmol) through the septum. Reaction mixture was heated to 110° C. overnight. Then was mixture acidified to pH=2 with 1 M aqueous solution of hydrochloric acid and extracted with ethyl acetate. After drying over anhydrous sodium sulfate and filtration was solvent evaporated under reduced pressure. Residue was purified by column chromatography (Silicagel 60, 0.063-0.200 mm; eluent:cyclohexane) to give 3-bromo-5-(trifluoromethyl)benzenethiol (2) as white oil.

Yield: 5.76 g (52%). ¹H NMR spectrum (300 MHz, CDCl₃, δH): 7.61 (s, 1H); 7.56 (s, 1H); 7.46 (s, 1H); 3.66 (s, 1H).

3-Bromo-5-(trifluoromethyl)benzenethiol (2, 5.76 g, 22.4 mmol), methyl 3-bromo-5-iodobenzoate (3, 5.09 g, 14.9 mmol), potassium carbonate (2.95 g, 24.8 mmol) and copper(I) iodide (410 mg, 2.49 mmol) were dissolved in dry dimethoxyethane (44 mL). Reaction flask was heated to 80° C. for 48 hours. After this time was mixture diluted with ethyl acetate and filtrated through the celite, solvent was then evaporated under reduced pressure. Residue was purified by column chromatography (Silicagel 60, 0.063-0.200 mm; eluent: cyclohexane/ethyl acetate 1:0 to 20:1) to give methyl 3-bromo-5-((3-bromo-5-(trifluoromethyl)phenyl)thio)benzoate (4) as yellow oil. Yield: 6.64 g (63%). ¹H NMR spectrum (300 MHz, CDCl₃, δH): 7.82 (m, 1H); 7.68 (m, 3H); 7.52 (m, 1H); 2.54 (s, 3H).

Methyl 3-bromo-5-((3-bromo-5-(trifluoromethyl)phenyl)thio)benzoate (4, 6.64 g, 14.1 mmol) and Oxone (8.20 g, 35.3 mmol) were suspended in methanol (30 mL) and water (10 mL) was added. The reaction was stirred overnight at room temperature. Then was mixture diluted with ethyl acetate (50 mL), washed with water (1 L) and then with brine (100 mL). Organic phase was evaporated under reduced pressure, residue was chromatographed by column chromatography (Silicagel 60, 0.063-0.200 mm; eluent:cyclohexane/ethyl acetate 9:1 to 3:1) to give methyl 3-bromo-5-((3-bromo-5-(trifluoromethyl)phenyl)sunfonyl)benzoate (5) as white solid. Yield: 5.46 g (77%). ¹H NMR spectrum (300 MHz, CDCl₃, δH): 8.53 (m, 1H); 8.43 (m, 1H); 8.27 (m, 2H); 8.15 (m, 1H); 8.00 (m, 1H); 4.00 (s, 3H).

Methyl 3-bromo-5-((3-bromo-5-(trifluoromethyl)phenyl)sunfonyl)benzoate (5, 5.46 g 10.9 mmol) and lithium hydroxide monohydrate (1.33 g, 31.7 mmol) were dissolved in mixture of methanol/water/tetrahydrofuran (4:2:5, 35 mL), reaction mixture was stirred overnight at room temperature. After this time was mixture acidified to pH 2 with 1 M aqueous solution of hydrochloric acid and extracted with ethyl acetate. After evaporation of all volatiles was obtained 3-bromo-5-((3-bromo-5-(trifluoromethyl)phenyl)sunfonyl)benzoic acid (6) as white solid. Yield: 5.10 g (96%). ¹H NMR spectrum (300 MHz, DMSO-d6 δH): 13.91 (bs, 1H); 8.68 (s, 2H); 8.50 (s, 1H); 8.45 (s, 1H); 8.41 (s, 1H); 8.32 (s, 1H).

3-Bromo-5-((3-bromo-5-(trifluoromethyl)phenyl)sunfonyl)benzoic acid (6, 5.10 g, 10.5 mmol) mixed with 1-((dimethylamino)(dimethyliminio)methyl)-1H-[1,2,3]triazolo[4,5-b]pyridine 3-oxide hexafluorophosphate(V) (HATU, 4.40 g, 11.6 mmol) in dry N,N-dimethylformamide (130 mL) was stirred for 30 minutes, then triethylamine (7.5 ml, 52.3 mmol) was added and glycine tert-butyl ester hydrochloride (3.51 g, 20.9 mmol) were added and stirred overnight. After end of reaction was added water and reaction mixture was extracted with ethyl acetate (150 mL), after evaporation of all volatiles under reduced pressure was residue purified by column chromatography (Silicagel 60, 0.063-0.200 mm; eluent:cyclohexane/ethyl acetate 3:1) to give tert-butyl (3-bromo-5-((3-bromo-5-(trifluoromethyl)phenyl)sulfonyl)benzoyl)glycinate (7) as white solid, Yield: 6.30 g (99%). LC-MS: 602.3 (M+H)+.

A 100 mL reaction flask was charged with potassium acetate (5.13 g, 26.1 mmol) and the salt was dried for 1 hour at 110° C. in vacuo. After cooling to room temperature, the reaction flask was backfilled with nitrogen and charged with tert-butyl (3-bromo-5-((3-bromo-5-(trifluoromethyl)phenyl)sulfonyl)benzoyl)glycinate (7, 6.30 g, 10.5 mmol), palladium acetate (120 mg, 0.52 mmol), 2-dicyclohexylphosphino-2,4,6-triisopropylbiphenyl (XPhos, 500 mg, 1.04 mmol) and bis(pinacolato)diboron (5.9 g, 23.03 mmol). The reaction flask was then evacuated and backfilled with nitrogen (this procedure was repeated twice), anhydrous tetrahydrofuran (50 mL) was added with syringe, the flask was sealed with a plastic stopper and heated to 60 C. Reaction mixture was stirred overnight and then was cooled to ambient temperature, diluted with dichloromethane (150 mL) and filtered through a short plug of silicagel topped with celite and washed with dichloromethane (3×50 mL). The filtrate was concentrated under reduced pressure to afford the tert-butyl (3-(4,4,5,5-tetramethyl-1,3,2-dioxaboroloan-2-yl)-5-((3-(4,4,5,5-tetramethyl-1,3,2-dioxaborolan-2-yl)-5-(trifluoromethyl)phenyl)sulfonyl)benzoyl)glycinate (8) as black waxy foam. Yield: 6.70 g (92%). LC-MS: 640.5 (M+H-tBu)+, 558.4 (M−pinacol−tBu+H)+, 476.3 (M−2pinacol−tBu+H)+.

tert-Butyl (3-(4,4,5,5-tetramethyl-1,3,2-dioxaboroloan-2-yl)-5-((3-(4,4,5,5-tetramethyl-1,3,2-dioxaborolan-2-yl)-5-(trifluoromethyl)phenyl)sulfonyl)benzoyl)glycinate (8, 6.70 g, 10.5 mmol) was mixed with trifluoroacetic acid (25 mL) and stirred 1 hour at room temperature, after this time all volatiles was evaporated under reduced pressure. Residue was then dissolved in ethyl acetate (50 mL) and filtered through a short plug of silicagel topped with celite. The filtrate was concentrated under reduced pressure, was obtained orange hard foam, which was crushed. (3-(4,4,5,5-tetramethyl-1,3,2-dioxaboroloan-2-yl)-5-((3-(4,4,5,5-tetramethyl-1,3,2-dioxaborolan-2-yl)-5-(trifluoromethyl)phenyl)sulfonyl)benzoyl)glycine (9) was obtained as pale orange solid. Yield: 3.89 g (63%). ¹H NMR spectrum (300 MHz, CDCl₃, δH): 8.57 (m, 3H); 8.47 (s, 1H); 8.31 (s, 1H); 9.26 (s, 1H); 7.23 (t, 1H); 4.35 (d, 2H); 1.38 (s, 1H). ¹⁹F NMR spectrum (282 MHz, CDCl₃, δF): −62.65 (s). LC-MS: 640.5 (M+H)+, 558.4 (M−pinacol+H)+, 476.3 (M−2×pinacol+H)+.

### Example 23: N-5-Fluoro-1-hydroxy-1,3-dihydrobenzo[c][1,2]oxaborole-6-carbonyl-N-2-(5-fluoro-1-hydroxy-1,3-dihydrobenzo[c][1,2]oxaborole-6-carboxamido)ethyl)glycine

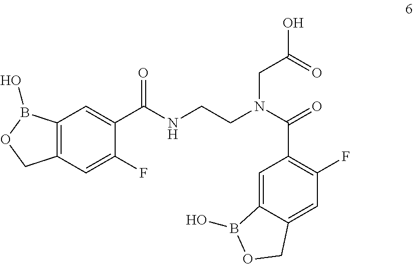 [SMILES: O=C(O)CN(CCNC(=O)c1cc2c(cc1F)COB2O)C(=O)c1cc2c(cc1F)COB2O]

Chloroacetic acid (1, 13.0 g, 136 mmol) was added to precooled (0° C.) ethylenediamine (2, 90 mL) in small portions. After the addition was complete, the reaction mixture was allowed to reach room temperature overnight (16 hours). Ethylenediamine was evaporated in vacuo and the residue was triturated with dimethyl sulfoxide (140 mL) with stirring overnight. The precipitate was collected by filtration and washed by dimethyl sulfoxide (2×60 mL), acetonitrile (3×100 mL) and diethyl ether (3×100 mL) to give the (2-aminoethyl)glycine (3) as colorless solid. Yield: 13.2 g (83%). ¹H NMR spectrum (300 MHz, D2O, δH): 3.27 (s, 2H); 3.05-3.01 (m, 2H); 2.92-2.88 (m, 2H).

Solution of 2,3,4,5,6-pentafluorophenol (9.39 g, 51.0 mmol), 5-fluoro-1-hydroxy-1,3-dihydrobenzo[c][1,2]oxaborole-6-carboxylic acid (4, 10.0 g, 51.0 mmol) and N,N′-dicyclohexylcarbodiimide (DCC, 10.5 g, 51.0 mmol) in acetonitrile (300 mL) was stirred at ambient temperature overnight. The reaction mixture was filtered, washed with acetonitrile and evaporated. Crude product 5 was purified by crystallization from mixture of dichloromethane/hexane (9:1, 500 mL) to give the pentafluorophenyl 5-fluoro-1-hydroxy-1,3-dihydrobenzo[c][1,2]oxaborole-6-carboxylate (5) as white solid. Yield: 6.80 g (37%). LC-MS: 363.2 (M+H)+.

Solution of pentafluorophenyl 5-fluoro-1-hydroxy-1,3-dihydrobenzo[c][1,2]oxaborole-6-carboxylate (5, 6.80 g, 18.8 mmol), (2-aminoethyl)glycine (3, 1.10 g, 9.39 mmol) and triethylenamine (10.5 mL, 75.1 mmol) in N,N-dimethylformamide (80 mL) was stirred at ambient temperature overnight. The reaction mixture was evaporated and extracted with ethyl acetate (2×500 mL) and 1 M aqueous solution of hydrochloric acid (400 mL), organic layers were washed with brine (300 mL). The organic phase was dried over anhydrous sodium sulfate, filtered and evaporated. Crude product 6 was purified by flash chromatography (Silicagel, 0.063-0.200 mm; eluent:dichloromethane/methanol/formic acid 100:2:0.5 to 100:10:0.5) and freeze-dried to afford N-(5-fluoro-1-hydroxy-1,3-dihydrobenzo[c][1,2]oxaborole-6-carbonyl)-N-(2-(5-fluoro-1-hydroxy-1,3-dihydrobenzo[c][1,2]oxaborole-6-carboxamido)ethyl)glycine (6) as white solid.

Yield: 2.16 g (49%). RF (SiO2, dichloromethane/methanol/formic acid 100:2:0.5): 0.30.

¹H NMR spectrum (300 MHz, DMSO-d6, δH): 12.86 (bs, 1H); 9.45-9.17 (m, 2H); 8.48-8.11 (m, 1H); 8.11-7.88 (m, 1H); 7.65 (d, J=7.0 Hz, 1H); 7.43-7.15 (m, 2H); 4.99 (d, J=8.6 Hz, 4H); 4.36-3.90 (m, 2H); 3.80-3.34 (m, 4H). LC-MS: 475.4 (M+H)+.

### Example 24: 4-((3S,4S)-3,4-Bis(1-hydroxy-4-(trifluoromethyl)-1,3-dihydrobenzo[c][1,2]oxaborole-6-carboxamido)pyrrolidin-1-yl)-4-oxobutanoic acid

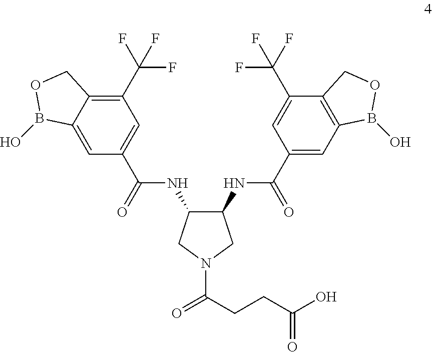 [SMILES: O=C(O)CCC(=O)N1C[C@H](NC(=O)c2cc3c(c(C(F)(F)F)c2)COB3O)[C@@H](NC(=O)c2cc3c(c(C(F)(F)F)c2)COB3O)C1]

1-Hydroxy-4-(trifluoromethyl)-1,3-dihydrobenzo[c][1,2]oxaborole-6-carboxylic acid (1, 13.5 g, 54.9 mmol), N-hydroxysuccinimide (6.31 g, 54.9 mmol) and 1-ethyl-3-(3′-dimethylaminopropyl) carbodiimide hydrochloride (10.5 g, 54.9 mmol) were stirred in tetrahydrofuran (270 mL) and N,N-dimethylformamide (40 mL) for 4 hours at ambient temperature. The reaction mixture was evaporated and extracted with ethyl acetate (3×300 mL) and 1 M aqueous solution of hydrochloric acid (200 mL). The organic phase was dried over anhydrous sodium sulfate, filtered and evaporated to afford 2,5-dioxopyrrolidin-1-yl 1-hydroxy-4-(trifluoromethyl)-1,3-dihydrobenzo[c][1,2]oxaborole-6-carboxylate (2) as white solid. Yield: 18.8 g (100%). LC-MS: 344.3 (M+H)+.

Solution of 4-((3S,4S)-3,4-diaminopyrrolidin-1-yl)-4-oxobutanoic acid dihydrochloride (3, 2.74 mg, 10.0 mmol), 2,5-dioxopyrrolidin-1-yl 1-hydroxy-4-(trifluoromethyl)-1,3-dihydrobenzo[c][1,2]oxaborole-6-carboxylate (2, 6.86 mg, 20.0 mmol) and N,N-diisopropylethylamine (11.0 mL, 60.0 mmol) in N,N-dimethylformamide (240 mL) and water (60 mL) was stirred at ambient temperature overnight. The reaction mixture was evaporated, purified by column chromatography (Silicagel, 0.063-0.200 mm; eluent: dichloromethane/methanol/formic acid 100:2:0.5 to 100:10:0.5) and freeze-dried to afford 4-((3S,4S)-3,4-bis(1-hydroxy-4-(trifluoromethyl)-1,3-dihydrobenzo[c][1,2]oxaborole-6-carboxamido)pyrrolidin-1-yl)-4-oxobutanoic acid (4) as white solid.

Yield: 2.56 g (39%). Rf (SiO2, dichloromethane/methanol/formic acid 100:10:0.5): 0.3.

¹H NMR spectrum (300 MHz, DMSO-d6, δH) 11.73 (bs, 1H); 9.62 (s, 2H); 9.01 (dd, J=10.4 and 7.2 Hz, 2H); 8.49 (s, 2H); 8.24 (s, 2H); 5.20 (s, 4H); 4.94-4.51 (m, 2H); 4.16-3.94 (m, 1H); 3.95-3.80 (m, 1H); 3.56-3.44 (m, 1H); 3.41-3.34 (m, 1H); 2.49-2.40 (m, 4H). LC-MS: 658.7 (M+H)+.

### Example 25: 2,5-Dioxopyrrolidin-1-yl 3-(difluoro(3-(4,4,5,5-tetramethyl-1,3,2-dioxaborolan-2-yl)-5-(trifluoromethyl)phenyl)methyl)-5-(4,4,5,5-tetramethyl-1,3,2-dioxaborolan-2-yl)benzoate

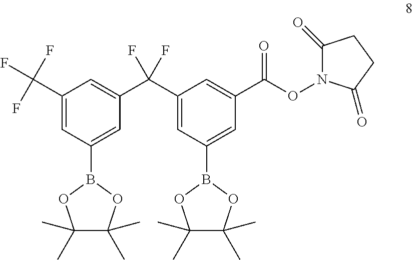 [SMILES: CC1(C)OB(c2cc(C(=O)ON3C(=O)CCC3=O)cc(C(F)(F)c3cc(B4OC(C)(C)C(C)(C)O4)cc(C(F)(F)F)c3)c2)OC1(C)C]

Methyl 3-bromo-5-iodobenzoate (1, 6.80 g, 20.0 mmol) was dissolved in dry tetrahydrofuran (40 mL) and cooled to −30 C. 1.3 M Solution isopropylmagnesium chloride-lithium chloride complex in tetrahydrofuran (16.2 mL, 21.0 mmol) was added dropwise with stirring. After 30 minutes, 3-bromo-5-(trifluoromethyl)benzaldehyde (2, 6.00 g, 24.0 mmol) was added with aid of tetrahydrofuran (10 mL). The resulting mixture was allowed to warm to ambient temperature and quenched after one hour by the addition of 1 M aqueous solution of hydrochloric acid (40 mL). The reaction mixture was taken up in diethyl ether (150 mL), washed with water (150 mL) and brine (100 mL), dried over anhydrous sodium sulfate, filtered and evaporated. The crude product (3) was dissolved in dry dichloromethane (80 mL) and pyridinium chlorochromate (6.42 g, 30.0 mmol) was added with stirring. After stirring for 17 hours, the reaction mixture was filtered through a plug of silica (80 g) topped with celite and the bed was washed with dichloromethane (3×120 mL). The yellowish solution was concentrated in vacuo and the residue stirred in methanol (50 mL) for 16 hours. The precipitated solid was collected by filtration and dried in air to give methyl 3-bromo-5-(3-bromo-5-(trifluoromethyl)benzoyl)benzoate (4) as colorless solid. Yield: 6.52 g (70%).

¹H NMR spectrum (300 MHz, CDCl₃, δH): 8.45 (t, J=1.4 Hz, 1H); 8.29 (m, 1H); 8.12 (t, J=1.6 Hz, 1H); 8.08 (bs, 1H); 8.04 (bs, 1H); 7.95 (bs, 1H); 3.97 (s, 3H).

Methyl 3-bromo-5-(3-bromo-5-(trifluoromethyl)benzoyl)benzoate (4, 6.50 g, 13.9 mmol, and Deoxo-Fluor (13.0 mL) were charged to a 100 mL reaction vessel. The vessel was sealed with a bubbler (filled with silicon oil), purged with nitrogen and heated to 90° C. (oil bath) for 16 hours. The reaction mixture was cooled to ambient temperature and diluted with dichloromethane (100 mL). The resulting solution was added slowly to a 1 M aqueous potassium carbonate solution (100 mL) and the biphasic mixture was stirred for an hour to decompose the excess of fluorinating reagent. The layers were separated and the organic layer was dried over anhydrous sodium sulfate, filtered and evaporated. The crude product was purified by flash column chromatography (Silicagel 60, 0.040-0.063 mm; eluent: cyclohexane/ethyl acetate 30:1 to 15:1) to give methyl 3-bromo-5-((3bromo5(trifluoromethyl)phenyl)difluoromethyl) benzoate (5) as yellowish oil. Yield: 6902 mg (99%). ¹H NMR spectrum (300 MHz, CDCl₃, δH): 8.30 (s, 1H); 8.08 (s, 1H); 7.88 (s, 1H); 7.83 (s, 1H); 7.81 (s, 1H); 7.71 (s, 1H); 3.96 (s, 3H). ¹⁹F NMR spectrum (282 MHz, CDCl₃, δF): −62.87 (s, 3H); −90.00 (s, 2H).

A 500 mL reaction vessel was charged with potassium acetate (6.83 g, 69.7 mmol) and the salt was dried for 1 hour at 110° C. in vacuo. After cooling to room temperature, the reaction vessel was backfilled with nitrogen and charged with 3-bromo-5-((3bromo5(trifluoromethyl)phenyl)difluoromethyl) benzoate (5, 6.90 g, 13.9 mmol), palladium acetate (62.0 mg, 279 mol), 2-dicyclohexylphosphino-2,4,6-triisopropylbiphenyl (XPhos, 265 mg, 557 mol) and bis(pinacolato)diboron (838 mg, 30.7 mmol). The reaction vessel was then evacuated and backfilled with nitrogen (this procedure was repeated twice). Anhydrous tetrahydrofuran (50 mL) was added with syringe, the vessel was sealed with a plastic stopper and submerged in the heating bath preheated to 60 C. After stirring at 400 rpm for 16 hours the reaction mixture was cooled to ambient temperature, diluted with dichloromethane (200 mL) and filtered through a short plug of silica (90 g) topped with celite S with the aid of dichloromethane (3×120 mL). The filtrate was concentrated under reduced pressure to afford methyl 3-(difluoro(3-(4,4,5,5-tetramethyl-1,3,2-dioxaborolan-2-yl)-5-(trifluoromethyl)phenyl)methyl)-5-(4,4,5,5-tetramethyl-1,3,2-dioxaborolan-2-yl)benzoate (6) as brownish foam. It was suspended in methanol (50 mL) and water (15 mL) and lithium hydroxide monohydrate (2.94 g, 70.0 mmol) was added and the resulting mixture was stirred for 16 hours at room temperature. The reaction mixture was taken up in water (150 mL) and washed with dichloromethane (2×30 mL) and diethyl ether (30 mL). The aqueous layer was acidified by concentrated aqueous hydrochloric acid to pH=2 and extracted with ethyl acetate (100 mL). The organic layer was washed with brine (50 mL) and dried over anhydrous sodium sulfate, filtered and concentrated under reduced pressure to give a yellowish foam. To the foam was added pinacol (472 mg, 4.00 mmol) and left to stir overnight in acetonitrile (50 mL). The precipitated solid was collected by filtration, washed with ice-cold acetonitrile (2×20 mL) and dried in air to give the title 3-(difluoro(3-(4,4,5,5-tetramethyl-1,3,2-dioxaborolan-2-yl)-5-(trifluoromethyl)phenyl)methyl)-5-(4,4,5,5-tetramethyl-1,3,2-dioxaborolan-2-yl)benzoic acid (7) as colorless solid. Yield: 5.90 g (77%). ¹H NMR spectrum (300 MHz, CDCl₃, δH): 8.64 (s, 1H); 8.28 (s, 1H); 8.22 (s, 1H); 8.15 (s, 2H); 7.85 (s, 1H); 1.38 (s, 12H); 1.37 (s, 12H). LC-MS: 569.7 (M+H)+.

3-(Difluoro(3-(4,4,5,5-tetramethyl-1,3,2-dioxaborolan-2-yl)-5-(trifluoromethyl)phenyl)methyl)-5-(4,4,5,5-tetramethyl-1,3,2-dioxaborolan-2-yl)benzoic acid (7, 5.11 g, 9.00 mmol) and bis(succinimidyl)carbonate (3.22 g, 12.6 mmol) were suspended in anhydrous acetonitrile (45 mL) under nitrogen and pyridine (1.00 mL, 12.6 mmol). The reaction mixture was heated gently with a heatgun to effect dissolution. After stirring for 16 hours, the reaction mixture was concentrated in vacuo and the residue was taken up in ethyl acetate (100 mL) and washed with 0.5 M aqueous solution of potassium hydrogencarbonate (2×40 mL) and brine (50 mL). The organic layer was dried over anhydrous sodium sulfate, filtered and concentrated under reduced pressure to give an off-white solid. Pinacol (354 mg, 3.00 mmol) was added and mixture was left to stir overnight in acetonitrile (50 mL). The precipitated solid was collected by filtration, washed with ice-cold acetonitrile (2×20 mL) and dried in air to give the title 2,5-dioxopyrrolidin-1-yl 3-(difluoro(3-(4,4,5,5-tetramethyl-1,3,2-dioxaborolan-2-yl)-5-(trifluoromethyl)phenyl)methyl)-5-(4,4,5,5-tetramethyl-1,3,2-dioxaborolan-2-yl)benzoate (8) as colorless solid. Yield: 5.36 g (90%). ¹H NMR spectrum (300 MHz, CDCl₃, δH): 8.67 (s, 1H); 8.29 (s, 1H); 8.26 (s, 1H); 8.15 (s, 1H); 8.10 (s, 1H); 7.86 (s, 1H); 2.92 (s, 4H); 1.36 (s, 24H). LC-MS: 646.8 (M−HF)+.

### Example 26: (S)-2,3-Bis(1-hydroxy-4-(trifluoromethyl)-1,3-dihydrobenzo[c][1,2]oxaborole-6-carboxamido)propanoic acid

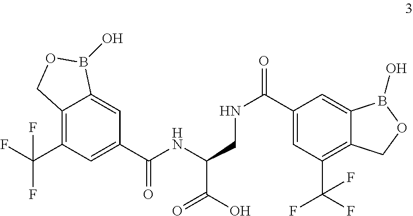 [SMILES: O=C(NC[C@H](NC(=O)c1cc2c(c(C(F)(F)F)c1)COB2O)C(=O)O)c1cc2c(c(C(F)(F)F)c1)COB2O]

Solution of 2,5-dioxopyrrolidin-1-yl 1-hydroxy-4-(trifluoromethyl)-1,3-dihydrobenzo[c][1,2]oxaborole-6-carboxylate (1, 14.4 g, 42.0 mmol), (S)-2,3-diaminopropanoic acid hydrochloride (2, 2.81 g, 20.0 mmol) and N,N-diisopropylethylamine (21.4 mL, 120 mmol) in N,N-dimethylformamide (400 mL) and water (100 mL) was stirred at ambient temperature overnight. The reaction mixture was evaporated and purified by column chromatography (Silicagel, 0.063-0.200 mm; eluent:dichloromethane/methanol/formic acid 100:2:0.5 to 100:10:0.5). The fractions with desired product were evaporated and washed with 1 M aqueous solution of potassium bisulfate (400 mL). The precipitate was filtered, dissolved in mixture of acetonitrile and water (2:1) and freeze-dried to afford (S)-2,3-bis(1-hydroxy-4-(trifluoromethyl)-1,3-dihydrobenzo[c][1,2]oxaborole-6-carboxamido)propanoic acid (3) as white solid. Yield: 4.32 g (39%). ¹H NMR spectrum (300 MHz, DMSO-d6, δH): 12.59 (bs, 1H); 9.62 (d, J=6.1 Hz, 2H); 9.09 (d, J=7.9 Hz, 1H); 8.98 (t, J=5.7 Hz, 1H); 8.50 (d, J=14.5 Hz, 2H); 8.24 (d, J=21.6 Hz, 2H); 5.20 (d, J=5.7 Hz, 4H); 4.87-4.58 (m, 1H); 4.02-3.80 (m, 1H); 3.79-3.54 (m, 1H). LC-MS: 561.6 (M+H)+.

### Example 27: (3-((3-(Pentafluoro-6-sulfanyl)-5-(4,4,5,5-tetramethyl-1,3,2-dioxaborolan-2-yl)phenyl)sulfonyl)-5-(4,4,5,5-tetramethyl-1,3,2-dioxaborolan-2-yl)benzoyl)glycine

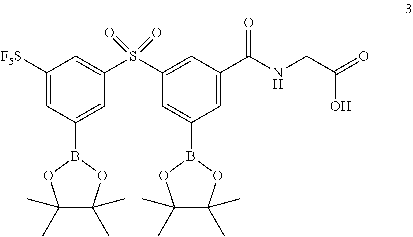

A mixture of tert-butyl (3-bromo-5-((3-bromo-5-(pentafluoro-6-sulfanyl)phenyl)sulfonyl)benzoyl)glycinate (1, 8.00 g, 12.1 mmol), palladium acetate (137 mg, 0.61 mmol), 2-dicyclohexylphosphino-2,4,6-triisopropylbiphenyl (XPhos, 577 mg, 1.21 mol), bis(pinacolato)diboron (6.78 g, 26.7 mmol) and potassium acetate (5.95 g, 60.7 mmol) in anhydrous tetrahydrofuran (450 mL) was heated under argon atmosphere at 60° C. for 24 hours. The mixture was cooled down to room temperature and filtered through a short plug of celite. Solvents were removed under reduced pressure and the residue was purified by flash column chromatography (Silicagel 60, 0.040-0.063 mm; eluent:dichloromethane/ethyl acetate 10:0 to 6:4) to give tert-butyl (3-((3-(pentafluoro-6-sulfanyl)-5-(4,4,5,5-tetramethyl-1,3,2-dioxaborolan-2-yl)phenyl)sulfonyl)-5-(4,4,5,5-tetramethyl-1,3,2-dioxaborolan-2-yl)benzoyl)glycinate (2) as off-white foam. Yield: 6.70 g (72%).

¹H NMR spectrum (300 MHz, CDCl₃, δH): 8.53-8.49 (m, 2H); 8.49-8.46 (m, 1H); 8.43 (t, J=1.9 Hz, 1H); 8.39-8.36 (m, 1H); 8.32 (dd, J=2.1 and 0.6 Hz, 1H); 6.76 (t, J=5.0 Hz, 1H); 4.16 (d, J=5.0 Hz, 2H); 1.51 (s, 9H); 1.36 (s, 24H). LC-MS: 754.9 (M+H)+.

A solution of tert-butyl (3-((3-(pentafluoro-6-sulfanyl)-5-(4,4,5,5-tetramethyl-1,3,2-dioxaborolan-2-yl)phenyl)sulfonyl)-5-(4,4,5,5-tetramethyl-1,3,2-dioxaborolan-2-yl)benzoyl)glycinate (2, 6.68 g, 8.87 mmol) in dichloromethane (100 mL) and trifluoroacetic acid (200 mL) was stirred at room temperature for 2 hours. Solvents were removed under reduced pressure. The residue was evaporated ten times from dichloromethane (250 mL) prior to drying in vacuo. (3-((3-(Pentafluoro-6-sulfanyl)-5-(4,4,5,5-tetramethyl-1,3,2-dioxaborolan-2-yl)phenyl)sulfonyl)-5-(4,4,5,5-tetramethyl-1,3,2-dioxaborolan-2-yl)benzoyl)glycine (3) was obtained as off-white solid. Yield: 6.15 g (99%). ¹H NMR spectrum (300 MHz, DMSO-d6, δH): 9.33 (t, J=5.8 Hz, 1H); 8.65 (t, J=1.8 Hz, 1H); 8.55 (t, J=1.9 Hz, 1H); 8.47 (s, 1H); 8.41-8.29 (m, 2H); 8.25-8.16 (m, 1H); 3.96 (d, J=5.9 Hz, 2H); 1.41-1.24 (m, 24H). LC-MS: 534.4 (M−2×pin+H)+.

### Example 28: N-(1-Hydroxy-4-(trifluoromethyl)-1,3-dihydrobenzo[c][1,2]oxaborole-6-carbonyl)-N-2-(1-hydroxy-4-(trifluoromethyl)-1,3-dihydrobenzo[c][1,2]oxaborole-6-carboxamido)ethyl)glycine

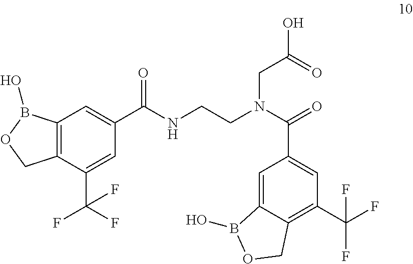 [SMILES: O=C(O)CN(CCNC(=O)c1cc2c(c(C(F)(F)F)c1)COB2O)C(=O)c1cc2c(c(C(F)(F)F)c1)COB2O]

1-Bromopyrrolidine-2,5-dione (NBS, 34.0 g, 191 mmol) was added to a solution of 3-trifluoromethyl-4-methylbenzoic acid (1, 39.0 g, 191 mmol) in concentrated sulfuric acid (400 mL) and the reaction mixture was stirred at ambient temperature for 16 hours. The reaction mixture was then poured into ice-water (2 L). Resulting precipitate was filtered off, washed with water (500 mL) and dissolved in ethyl acetate (400 mL); dried over anhydrous sodium sulfate, filtered and evaporated to provide 3-bromo-4-methyl-5-trifluoromethylbenzoic acid (2) as white solid. Yield: 53.4 g (98%). ¹H NMR spectrum (300 MHz, DMSO-d6, δH): 13.71 (bs, 1H); 8.35 (d, J=0.4 Hz, 1H); 8.15 (d, J=0.9 Hz, 1H); 2.56 (s, 3H).

Concentrated sulfuric acid (24 mL) was added to a solution 3-bromo-4-methyl-5-trifluoromethylbenzoic acid (2, 35.0 g, 124 mmol) in methanol (500 mL) and the reaction mixture was allowed to stir under reflux for 4 hours and at ambient temperature for 16 hours. The reaction mixture was then evaporated under reduced pressure, dissolved in diethyl ether (250 mL), washed with water (2×100 mL) and mixture of saturated solution of potassium carbonate (100 mL) and brine (100 mL). Organic layer was separated, dried over anhydrous sodium sulfate, filtered and evaporated to provide methyl 3-bromo-4-methyl-5-trifluoromethylbenzoate (3) as white solid. Yield: 35.3 g (96%). ¹H NMR spectrum (300 MHz, DMSO-d6, δH): 8.36 (d, J=1.1 Hz, 1H); 8.13 (d, J=1.1 Hz, 1H); 3.90 (s, 3H); 2.55 (d, J=1.3 Hz, 3H).

A suspension of 1-bromopyrrolidine-2,5-dione (NBS, 31.7 g, 178 mmol) and methyl 3-bromo-4-methyl-5-trifluoromethylbenzoate (3, 35.3 g, 119 mmol) in water (300 mL) was stirred for 6 hours under 100 W light bulb at 80° C. Reaction mixture was extracted with diethyl ether (2×200 mL). Organic layers were washed with brine (150 mL). Organic layer was separated, dried over anhydrous sodium sulfate, filtered and evaporated to provide methyl 3-bromo-4-bromomethyl-5-trifluoromethylbenzoate (4) as yellow solid. Yield: 44.0 g (98%). ¹H NMR spectrum (300 MHz, CDCl₃, δH): 8.47 (d, J=1.5 Hz, 1H); 8.31 (d, J=1.3 Hz, 1H); 4.75 (s, 2H); 3.98 (s, 3H).

Solution of 3-bromo-4-bromomethyl-5-trifluoromethylbenzoate (4, 44.0 g, 117 mmol) and potassium acetate (22.9 g, 234 mmol) in acetonitrile (0.5 L) was stirred at 75° C. overnight. The suspension was filtered through filtering paper and evaporated. The crude product was dissolved in dichloromethane and filtered again. Evaporation provided methyl 3-bromo-4-(acetoxymethyl)-5-(trifluoromethyl)benzoate (5) as white solid. Yield: 37.9 g (91%). ¹H NMR spectrum (300 MHz, CDCl₃, δH): 8.49 (d, J=1.3 Hz, 1H); 8.34 (d, J=1.3 Hz, 1H); 5.37 (s, 2H); 3.99 (s, 3H); 2.11 (s, 3H).

Solution of methyl 3-bromo-4-(acetoxymethyl)-5-trifluoromethylbenzoate (5, 37.9 g, 107 mmol), bis(pinacolato)diboron (29.8 g, 117 mmol), potassium acetate (31.4 g, 294 mmol) and [1,1-bis(diphenylphosphino)ferrocene]dichloropalladium(II) (1.57 g, 1.92 mmol) in dry tetrahydrofuran (500 mL) was allowed to stir at 75° C. under argon atmosphere for 13 days. Then the reaction mixture was cooled to ambient temperature, filtered and evaporated. The crude product was filtered through silica gel column (Silicagel, 0.063-0.200 mm; eluent: cyclohexane/ethyl acetate 8:1) to provide methyl 4-(acetoxymethyl)-3-(4,4,5,5-tetramethyl-1,3,2-dioxaborolan-2-yl)-5-(trifluoromethyl)benzoate (6). Yield: 31.1 g (72%). RF (SiO2, cyclohexane/ethyl acetate 8:1): 0.40. ¹H NMR spectrum (300 MHz, CDCl₃, δH): 8.65 (s, 1H); 8.43 (s, 1H); 5.48 (s, 2H); 3.97 (s, 3H); 2.05 (s, 3H); 1.36 (s, 12H).

Solution of methyl 4-(acetoxymethyl)-3-(4,4,5,5-tetramethyl-1,3,2-dioxaborolan-2-yl)-5-(trifluoromethyl)benzoate (6, 31.0 g, 77.1 mmol) and sodium hydroxide (15.4 g, 386 mmol) in water (300 mL) was stirred at ambient temperature for 3 hours. Then solution of hydrochloric acid (35 mL) in water (100 mL) was added to lower the pH to 1. The reaction mixture was stirred overnight. Precipitate was filtered and dried to provide 1-hydroxy-4-(trifluoromethyl)-1,3-dihydrobenzo[c][1,2]oxaborole-6-carboxylic acid (7) as white solid. Yield: 16.6 g (86%).

¹H NMR spectrum (300 MHz, DMSO-d6, δH): 13.47 (bs, 1H); 9.66 (s, 1H); 8.62 (s, 1H); 8.24 (s, 1H); 5.22 (s, 2H).

Solution of pentafluorophenol (7.48 g, 40.7 mmol), 1-hydroxy-4-(trifluoromethyl)-1,3-dihydrobenzo[c][1,2]oxaborole-6-carboxylic acid (7, 10.0 mg, 40.7 mmol) and N,N′-dicyclohexylcarbodiimide (DCC, 8.37 mg, 40.7 mmol) in acetonitrile (0.5 L) was stirred at ambient temperature overnight. The reaction mixture was filtered, evaporated, dissolved in acetonitrile, re-filtered and evaporated to give the pentafluorophenyl 1-hydroxy-4-(trifluoromethyl)-1,3-dihydrobenzo[c][1,2]oxaborole-6-carboxylate (8) as white solid.

Yield: 16.7 g (100%). ¹H NMR spectrum (300 MHz, DMSO-d6, δH): 9.79 (s, 1H); 8.86 (s, 1H); 8.46 (s, 1H); 5.30 (s, 2H).

Solution of the pentafluorophenyl 1-hydroxy-4-(trifluoromethyl)-1,3-dihydrobenzo[c][1,2]oxaborole-6-carboxylate (8, 16.7 g, 40.6 mmol), (2-aminoethyl)glycine (9, 2.40 g, 20.3 mmol) and triethylamine (28.4 mL, 203 mmol) in N,N-dimethylformamide (0.5 L) was stirred at ambient temperature for 3 days. The reaction mixture was then evaporated and crude product 10 was purified by column chromatography (Silicagel, eluent: dichloromethane/methanol/formic acid 100:2:0.5 to 100:10:0.5) to give N-(1-hydroxy-4-(trifluoromethyl)-1,3-dihydrobenzo[c][1,2]oxaborole-6-carbonyl)-N-(2-(1-hydroxy-4-(trifluoromethyl)-1,3-dihydrobenzo[c][1,2]oxaborole-6-carboxamido)ethyl)glycine (10) as white solid. Yield: 7.77 g (67%). ¹H NMR spectrum (300 MHz, DMSO-d6, δH): 12.89 (bs, 1H); 9.68-9.48 (m, 2H); 9.00-8.67 (m, 1H); 8.56-7.36 (m, 4H); 5.27-5.03 (m, 4H); 4.30-3.95 (m, 2H); 3.77-3.48 (m, 4H). LC-MS: 575.5 (M+H)+.

### Example 29: (2S)-3-(2,3-Bis(1-hydroxy-4-(trifluoromethyl)-1,3dihydrobenzo[c][1,2]oxaborole-6-carboxamido)propanamido)propanoic acid=N-[N^{α},N^{β}-bis-(1-hydroxy-4-(trifluoromethyl)-1,3-dihydrobenzo[c][1,2]oxaborole-6-carboxamido)-L-diaminopropionyl]-β-alanine

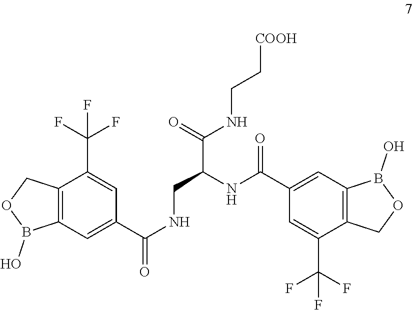 [SMILES: O=C(O)CCNC(=O)[C@H](CNC(=O)c1cc2c(c(C(F)(F)F)c1)COB2O)NC(=O)c1cc2c(c(C(F)(F)F)c1)COB2O]

A solution of L-diaminopropanoic acid hydrochloride alias (2S)-2,3-diaminopropanoic acid hydrochloride (1, 15.0 g, 107 mmol), di-tert-butyl dicarbonate (46.6 g, 214 mmol) and potassium bicarbonate (32.0 g, 320 mmol) in mixture of acetonitrile (400 mL) and water (400 mL) was stirred overnight. The solvent was removed under reduced pressure and the residue was acidified with saturated aqueous solution of potassium hydrogen sulfate until pH 1 was achieved. The reaction mixture was extracted with ethyl acetate (3×200 mL) and dried over anhydrous sodium sulfate. The solvent was removed under reduced pressure to give (2S)-2,3-bis((tert-butoxycarbonyl)amino)propanoic acid (2) as off-white solid. Yield: 28.2 g (87%). ¹H NMR spectrum (300 MHz, CDCl₃, δH): 5.85 (bs, 1H); 5.17 (bs, 1H); 4.31 (bs, 1H); 3.64-3.46 (m, 2H); 1.46 (s, 18H).

A solution of (2S)-2,3-bis((tert-butoxycarbonyl)amino)propanoic acid (2, 27.9 g, 91.7 mmol), tert-butyl 3-aminopropanoate (3, 16.7 g, 91.7 mmol), N-(3-dimethylaminopropyl)-N-ethylcarbodiimide hydrochloride (EDC.HCl, 21.1 g, 110 mmol), 1-hydroxy-7-azabenzotriazole (HOAt, 15.0 g, 110 mmol) and N,N-diisopropylethylamine (64.0 mL, 367 mmol) in dichloromethane (300 mL) was stirred overnight. The solvent was removed under reduced pressure; the residue was dissolved in ethyl acetate (600 mL), washed with 1 M aqueous solution of hydrochloric acid (4×300 mL) and saturated aqueous solution of sodium bicarbonate (4×300 mL) and dried over anhydrous sodium sulfate. The solvent was removed under reduced pressure to give tert-butyl (S)-3-(2,3-bis((tert-butoxycarbonyl)amino)propanamido)propanoate (4) as off-white solid. Yield: 36.1 g (91%).

¹H NMR spectrum (300 MHz, CDCl₃, δH): 7.01 (bs, 1H); 5.75 (bs, 1H); 5.14 (bs, 1H); 4.15 (bs, 1H); 3.57-3.39 (m, 4H); 2.43 (t, J=6.0 Hz, 2H); 1.45 (s, 27H).

To a solution of tert-butyl (S)-3-(2,3-bis((tert-butoxycarbonyl)amino)propanamido)propanoate (4, 36.1 g, 83.7 mmol) in dichloromethane (50 mL) was added 95% aqueous solution of trifluroacetic acid (300 mL) and the solution was stirred for 3 hours. The solvent was removed under reduced pressure and the residue was co-evaporated with acetonitrile (3×300 mL) and treated with 1 M solution of hydrogen chloride in dry diethyl ether (300 mL). The precipitate was filtered off and triturated with acetonitrile (2×600 mL) to give (2S)-3-(2,3-diaminopropanamido)propanoic acid dihydrochloride (5) as white powder.

Yield: 22.2 g (100%). ¹H NMR spectrum (300 MHz, D2O, δH): 4.35 (t, J=5.8 Hz, 1H); 3.63-3.46 (m, 4H); 2.67 (t, J=6.6 Hz, 2H).

Solution of pentafluorophenol (35.1 g, 191 mmol), 1-hydroxy-4-(trifluoromethyl)-1,3-dihydrobenzo[c][1,2]oxaborole-6-carboxylic acid (1, 40.8 g, 166 mmol, preparation as described in example 28) and N,N′-dicyclohexylcarbodiimide (DCC, 39.3 g, 191 mmol) in acetonitrile (1 L) was stirred at ambient temperature for 24 hours. The reaction mixture was filtered, evaporated, dissolved in acetonitrile, re-filtered and evaporated. The crude product was precipitated in dichloromethane (1 L) and filtered to give the pentafluorophenyl 1-hydroxy-4-(trifluoromethyl)-1,3-dihydrobenzo[c][1,2]oxaborole-6-carboxylate (6) as white solid. Yield: 52.8 g (77%). ¹H NMR spectrum (300 MHz, DMSO-d6, δH): 9.79 (s, 1H); 8.86 (s, 1H); 8.46 (s, 1H); 5.30 (s, 2H).

To a solution of (2S)-3-(2,3-diaminopropanamido)propanoic acid dihydrochloride (5, 6.41 g, 24.3 mmol) and triethylamine (33.8 mmol, 243 mmol) in water (50 mL) was added a solution of pentafluorophenyl 1-hydroxy-4-(trifluoromethyl)-1,3-dihydrobenzo[c][1,2]oxaborole-6-carboxylate (6, 20.0 g, 48.6 mmol) in 1,4-dioxane (100 mL) and the solution was stirred overnight. The reaction mixture was partitioned between ethyl acetate (300 mL) and 1 M aqueous solution of potassium hydrogen sulfate (1500 mL). The organic layer was washed with 1 M aqueous solution of potassium hydrogen sulfate (1×300 mL) and the solvent was removed under reduced pressure. The residue was triturated with diethyl ether (2×150 mL) and filtered. The solid was dissolved in 70% aqueous acetonitrile (600 mL) and freeze-dried to to give 3-(2(S),3-bis(1-hydroxy-4-(trifluoromethyl)-1,3-dihydrobenzo[c][1,2]oxaborole-6-carboxamido)propanamido)propanoic acid (7) as white powder. Yield: 12.1 g (80%).

¹H NMR spectrum (300 MHz, AcOD-d4, δH): 8.51 (s, 1H); 8.47 (s, 1H); 8.29 (s, 1H); 8.27 (s, 1H); 5.28 (s, 4H); 5.15 (t, J=6.1 Hz, 1H); 4.15-3.99 (m, 2H); 3.61 (t, J=6.4 Hz, 2H); 2.67 (t, J=6.3 Hz, 2H). LC-MS: 632.0 (M+H)+.

### Example 30: (S)-3-(2,3-Bis(4-fluoro-1-hydroxy-1,3-dihydrobenzo[c][1,2]oxaborole-6-carboxamido)propanamido)propanoic acid

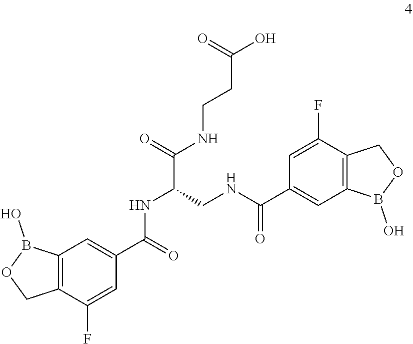 [SMILES: O=C(O)CCNC(=O)[C@H](CNC(=O)c1cc(F)c2c(c1)B(O)OC2)NC(=O)c1cc(F)c2c(c1)B(O)OC2]

4-Fluoro-1-hydroxy-1,3-dihydrobenzo[c][1,2]oxaborole-6-carboxylic acid (1, 8.56 g, 43.7 mmol), N-hydroxysuccinimide (5.03 g, 43.7 mmol) and 1-ethyl-3-(3′-dimethylaminopropyl) carbodiimide hydrochloride (8.38 g, 43.7 mmol) were stirred in tetrahydrofuran (250 mL) and N,N-dimethylformamide (20 mL) for 3.5 hours at ambient temperature. The reaction mixture was evaporated and extracted with ethyl acetate (3×150 mL) and 1 M aqueous solution of hydrochloric acid (150 mL). The organic phase was dried over anhydrous sodium sulfate, filtered and evaporated to afford 2,5-dioxopyrrolidin-1-yl 4-fluoro-1-hydroxy-1,3-dihydrobenzo[c][1,2]oxaborole-6-carboxylate (2) as white solid. Yield: 10.2 g (79%). LC-MS: 294.3 (M+H)+.

2-Chlorotrityl chloride resin 100-200 mesh 1.5 mmol/g (3, 10.5 g, 15.7 mmol) was left to swell in dry dichloromethane (80 mL) for 30 minutes. A solution of 3-((((9H-fluoren-9-yl)methoxy)carbonyl)amino)propanoic acid (Fmoc-Ala-OH, 3.26 g, 10.5 mmol) and N,N-diisopropylethylamine (6.93 mL, 39.8 mmol) in dry dichloromethane (50 mL) was added to resin and the mixture was shaken overnight. Resin was filtered and treated with a solution of N,N-diisopropylethylamine (3.65 mL, 20.9 mmol) in methanol/dichloromethane mixture (4:1, 2×5 min, 2×80 mL). Then resin was washed with N,N-dimethylformamide (2×80 mL), dichloromethane (2×80 mL) and N,N-dimethylformamide (3×80 mL). Fmoc group was removed by treatment with 20% piperidine in N,N-dimethylformamide (1×5 min, 1×20 min, 2×80 mL). Resin was washed with N,N-dimethylformamide (3×80 mL), 2-propanol (2×80 mL) and dichloromethane (3×80 mL). Solution of (S)-2,3-bis((((9H-fluoren-9-yl)methoxy)carbonyl)amino)propanoic acid (Fmoc-Dap(Fmoc)-OH, 8.61 g, 15.7 mmol), 5-chloro-1-((dimethylamino)(dimethyliminio)methyl)-1H-benzo[d][1,2,3]triazole 3-oxide tetrafluoroborate (TCTU, 5.58 g, 15.7 mmol) and N,N-diisopropylethylamine (4.92 mL, 28.2 mmol) in N,N-dimethylformamide (80 mL) was added to resin and mixture was shaken for 2 hours. Resin was filtered and washed with N,N-dimethylformamide (2×80 mL), dichloromethane (2×80 mL) and N,N-dimethylformamide (2×80 mL). Fmoc group was removed by treatment with 20% piperidine in N,N-dimethylformamide (1×5 min, 1×30 min, 2×80 mL). Resin was washed with N,N-dimethylformamide (3×80 mL), 2-propanol (2×80 mL) and dichloromethane (3×80 mL). Solution of 2,5-dioxopyrrolidin-1-yl 1-hydroxy-4-(trifluoromethyl)-1,3-dihydrobenzo[c][1,2]oxaborole-6-carboxylate (2, 9.14 g, 31.4 mmol) and N,N-diisopropylethylamine (9.84 mL, 56.5 mmol) in N,N-dimethylformamide (80 mL) was added to resin and mixture was shaken one day. Resin was filtered and washed with N,N-dimethylformamide (4×80 mL) and dichloromethane (10×80 mL). The product was cleaved from resin by treatment with 2,2,2-trifluoroethanol (80 mL) for 16 hours. Resin was filtered off and washed with dichloromethane (4×80 mL). Solvents were evaporated and crude product (4) was washed with ethyl acetate (300 mL), filtered and dried in vacuo. Pure product (4) was obtained as off-white solid. Yield: 4.10 g (74%). ¹H NMR spectrum (300 MHz, DMSO-d6, δH): 9.57 (bs, 2H); 8.78-8.49 (m, 2H); 8.19-7.93 (m, 3H); 7.71 (dd, J=30.8 and 10.8 Hz, 2H); 5.12 (d, J=7.7 Hz, 4H); 4.74-4.55 (m, 1H); 3.72-3.61 (m, 2H); 3.29-3.15 (m, 2H); 2.36 (t, J=6.9 Hz, 2H). LC-MS: 532.6 (M+H)+.

### Example 31: 4-((3R,4R)-3,4-Bis(7-fluoro-1-hydroxy-1,3-dihydrobenzo[c][1,2]oxaborole-6-carboxamido)pyrrolidin-1-yl)-4-oxobutanoic acid

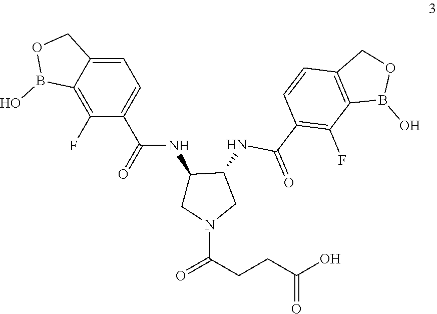 [SMILES: O=C(O)CCC(=O)N1C[C@@H](NC(=O)c2ccc3c(c2F)B(O)OC3)[C@H](NC(=O)c2ccc3c(c2F)B(O)OC3)C1]

Solution of 4-((3R,4R)-3,4-diaminopyrrolidin-1-yl)-4-oxobutanoic acid dihydrochloride (2, 2.46 g, 12.2 mmol), pentafluorophenyl 7-fluoro-1-hydroxy-1,3-dihydrobenzo[c][1,2]oxaborole-6-carboxylate (1, 8.86 g, 24.5 mmol) and triethylamine (17.0 mL, 122 mmol) in N,N-dimethylformamide (300 mL) was stirred at ambient temperature overnight. The reaction mixture was evaporated and precipitated from ethyl acetate to give 6.40 g of crude compound 3 (6.4 g), which was purified by HPLC (YMC, C18, 5 m, 250×50 mm, acetonitrile/water, 2:98 during 30 min, 2:98 to 30:0 during 180 min) and freeze-dried to give title compound 4-((3R,4R)-3,4-bis(7-fluoro-1-hydroxy-1,3-dihydrobenzo[c][1,2]oxaborole-6-carboxamido)pyrrolidin-1-yl)-4-oxobutanoic acid (3) as white solid. Yield: 1.23 g (18%).

¹H NMR spectrum (300 MHz, DMSO-d6, OH) 9.35 (bs, 2H); 8.68 (t, J=8.4 Hz, 2H); 7.81-7.62 (m, 2H); 7.30 (d, J=7.3 Hz, 2H); 5.02 (s, 4H); 4.66-4.45 (m, 2H); 3.94 (dd, J=10.6 and 6.7 Hz, 1H); 3.77 (dd, J=12.0 and 6.7 Hz, 1H); 3.54-3.41 (m, 1H); 3.27-3.18 (m, 1H); 2.47-2.34 (m, 4H). LC-MS: 558.6 (M+H)+.

### Example 32: N-(7-Fluoro-1-hydroxy-1,3-dihydrobenzo[c][1,2]oxaborole-6-carbonyl)-N-(2-(7-fluoro-1-hydroxy-1,3-dihydrobenzo[c][1,2]oxaborole-6-carboxamido)ethyl)glycine

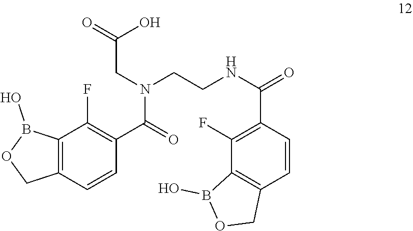 [SMILES: O=C(O)CN(CCNC(=O)c1ccc2c(c1F)B(O)OC2)C(=O)c1ccc2c(c1F)B(O)OC2]

n-Butyllithium (2.38 M in hexanes, 107 mL, 255 mmol) was cannulated to a stirred nitrogen purged solution of 2,2,6,6-tetramethylpiperidine (43.5 mL, 257 mmol) in anhydrous tetrahydrofuran (150 mL) at a such rate to maintain the internal temperature below −60° C. (ca 20 minutes). The mixture was stirred for 60 minutes (internal temperature increased to −40 C). The mixture was re-cooled to −78° C. and a solution of 2-fluoro-4-methylbenzonitrile (1, 30.0 g, 222 mmol) in dry tetrahydrofuran (200 mL) was added dropwise via peristaltic pump to the vigorously stirred mixture at a such rate to keep the internal temperature below −70° C. (ca 40 minutes). The mixture was warmed up to −50° C. and kept at this temperature for 45 minutes. The mixture was re-cooled to −78° C. and a solution of iodine (62.0 g, 244 mmol) in dry tetrahydrofuran (150 mL) was added dropwise (using peristaltic pump) to the reaction mixture while keeping internal temperature below −70 C. The residual iodine was washed with dry tetrahydrofuran (50 mL) and the mixture was stirred at −70° C. for 1 hour. The stirred mixture was left to warm up to room temperature overnight and then it was quenched by pouring to a stirred solution of sodium thiosulfate (20 g) in water (750 mL). The reaction mixture was stirred for 1 hour and then it was extracted with ethyl acetate (3×300 mL). The combined organic extracts were dried over anhydrous sodium sulfate and evaporated under reduced pressure. The residue was purified by column chromatography (Silicagel 60, 0.063-0.200 mm; eluent:cyclohexane/ethyl acetate 10:1) and then it was recrystallized from methanol to afford 2-fluoro-3-iodo-4-methylbenzonitrile (2) as a colorless crystalline solid.

Yield: 29.6 g (51%). RF (SiO2, cyclohexane/ethyl acetate 10:1): 0.35. ¹H NMR spectrum (300 MHz, CDCl₃, δH): 7.48 (dd, J=7.9 and 6.5 Hz, 1H); 7.17-7.12 (m, 1H), 2.56 (s, 3H). ¹⁹F NMR spectrum (282 MHz, CDCl₃, δF): −82.34 (s).

A slurry of 2-fluoro-3-iodo-4-methylbenzonitrile (2, 52.7 g, 202 mmol) in 75% sulfuric acid (65 mL) was stirred at 150° C. for 3 hours. After cooling to ambient temperature, the mixture was poured on ice/water mixture (500 g). The precipitated beige solid was filtered off, washed with copious amount of water and dried to yield 2-fluoro-3-iodo-4-methylbenzoic acid (3) as a beige solid. Yield: 51.2 g (91%). ¹H NMR spectrum (300 MHz, DMSO-d6, δH): 13.30 (s, 1H); 7.75 (t, J=7.8 Hz, 1H); 7.27 (d, J=8.0 Hz, 1H); 2.47 (s, 3H).

Acetyl chloride (23.0 mL, 321 mmol) was added dropwise to a stirred suspension of 2-fluoro-3-iodo-4-methylbenzoic acid (3, 90.0 g, 321 mmol) in dry methanol (350 mL) at 0 C. The mixture was refluxed overnight. The volatiles were removed under reduced pressure and the residue was taken up in ethyl acetate (1300 mL). After washing with saturated aqueous solution of potassium bicarbonate (2×1000 mL) and brine (1000 mL), the organic layer was dried over anhydrous magnesium sulfate and evaporated in vacuo. The residue was purified by column chromatography (Silicagel 60, 0.063-0.200 mm; eluent:cyclohexane/ethyl acetate 30:1-15:1) to give methyl 2-fluoro-3-iodo-4-methylbenzoate (4) as a colorless solid.

Yield: 67.6 g (72%). RF (SiO2, cyclohexane/ethyl acetate 15:1): 0.40. ¹H NMR spectrum (300 MHz, CDCl₃, δH): 7.80 (t, J=7.7 Hz, 1H); 7.10 (d, J=8.0 Hz, 1H); 3.93 (s, 3H); 2.52 (s, 3H).

A solution of 2-fluoro-3-iodo-4-methylbenzoate (4, 35.0 g, 119 mmol), bis(pinacolato)diboron (5, 33.3 g, 131 mmol), anhydrous potassium acetate (35.0 g, 357 mmol) and [1,1-bis(diphenylphosphino)ferrocene]dichloropalladium(II) complex with dichloromethane (1.94 g, 2.38 mmol) in anhydrous dimethylsulfoxide (500 mL) was stirred at 110° C. under argon atmosphere over weekend. The reaction mixture was cooled to ambient temperature, solvent was evaporated in vacuo and the crude product 6 was extracted with ethyl acetate (4×500 mL) and water (1.0 L). Organic layers were combined, filtered through a celite pad, dried over anhydrous sodium sulfate, filtered and concentrated under reduced pressure. The crude product was purified by flash chromatography (Silicagel 60, 0.063-0.200 mm; eluent: cyclohexane/ethyl acetate 9:1) to provide methyl 2-fluoro-4-methyl-3-(4,4,5,5-tetramethyl-1,3,2-dioxaborolan-2-yl)benzoate (6) as a colorless solid. Yield: 29.4 g (84%). RF (SiO2, cyclohexane/ethyl acetate 9:1): 0.30. ¹H NMR spectrum (300 MHz, CDCl₃, δH): 7.84 (t, J=8.0 Hz, 1H); 7.00 (d, J=8.1 Hz, 1H); 3.90 (s, 3H); 2.47 (s, 3H); 1.39 (s, 12H).

A solution of methyl 2-fluoro-4-methyl-3-(4,4,5,5-tetramethyl-1,3,2-dioxaborolan-2-yl)benzoate (6, 27.5 g, 93.5 mmol), 1-bromopyrrolidine-2,5-dione (NBS, 18.3 g, 103 mmol) and 2,2-azobis(2-methylpropionitrile) (AIBN, 0.77 g, 4.68 mmol) in benzotrifluoride (300 mL) was stirred at 85° C. for 16 hours. The solvent was evaporated in vacuo and the residue was extracted with diethyl ether (2×150 mL). The organic layer was washed with water (100 mL) and brine (100 mL). Organic layer was dried over anhydrous sodium sulfate, filtered and concentrated under reduced pressure to give methyl 4-(bromomethyl)-2-fluoro-3-(4,4,5,5-tetramethyl-1,3,2-dioxaborolan-2-yl)benzoate (7) as a yellow solid. Yield: 33.5 g (96%).

¹H NMR spectrum (300 MHz, CDCl₃, δH): 7.93 (t, J=7.8 Hz, 1H); 7.21 (d, J=8.1 Hz, 1H); 4.71 (s, 2H); 3.91 (s, 3H); 1.42 (s, 12H).

A solution of methyl 4-(bromomethyl)-2-fluoro-3-(4,4,5,5-tetramethyl-1,3,2-dioxaborolan-2-yl)benzoate (7, 33.5 g, 89.8 mmol) and potassium acetate (17.6 g, 180 mmol) in acetonitrile (1 L) was stirred at 75° C. overnight. The suspension was filtered through cotton-wool and evaporated. The crude product was dissolved in dichloromethane and filtered again. Solvent was evaporated to give methyl 4-(acetoxymethyl)-2-fluoro-3-(4,4,5,5-tetramethyl-1,3,2-dioxaborolan-2-yl)benzoate (8) as a beige solid. Yield: 30.0 g (95%). ¹H NMR spectrum (300 MHz, CDCl₃, δH): 7.96 (t, J=7.8 Hz, 1H); 7.24 (d, J=7.9 Hz, 1H); 5.25 (s, 2H); 3.92 (s, 3H); 2.11 (s, 3H); 1.39 (s, 12H).

A solution of methyl methyl 4-(acetoxymethyl)-2-fluoro-3-(4,4,5,5-tetramethyl-1,3,2-dioxaborolan-2-yl)benzoate (8, 30.0 g, 85.2 mmol) and sodium hydroxide (17.0 g, 426 mmol) in water (250 mL) was stirred at ambient temperature for 3 hours. Afterwards, an aqueous solution of hydrochloric acid (35% w/w, 45 mL) in water (50 mL) was added to lower the pH to 1. The reaction mixture was stirred for 16 hours. The resulting precipitate was filtered and freeze dried to provide 7-fluoro-1-hydroxy-1,3-dihydrobenzo[c][1,2]oxaborole-6-carboxylic acid (9) as an off-white solid. Yield: 9.76 g (58%). ¹H NMR spectrum (300 MHz, DMSO-d6, δH): 13.17 (bs, 1H); 9.38 (bs, 1H); 8.29 (d, J=7.7 Hz, 1H); 7.36 (d, J=11.2 Hz, 1H); 5.02 (s, 2H). LC-MS: 197.3 (M+H)+.

A solution of 2,3,4,5,6-pentafluorophenol (9.61 g, 52.2 mmol), 7-fluoro-1-hydroxy-1,3-dihydrobenzo[c][1,2]oxaborole-6-carboxylic acid (9, 10.2 g, 52.2 mmol), and N,N′-dicyclohexylcarbodiimide (DCC, 10.8 g, 52.2 mmol) in acetonitrile (300 mL) and dichloromethane (200 mL) was stirred at ambient temperature over weekend. The reaction mixture was filtered and evaporated in vacuo. The residue was dissolved in acetonitrile, filtered and evaporated in vacuo again to give pentafluorophenyl 7-fluoro-1-hydroxy-1,3-dihydrobenzo[c][1,2]oxaborole-6-carboxylate (10) as a beige solid. Yield: 18.8 g (100%).

¹H NMR spectrum (300 MHz, DMSO-d6, δH): 9.55 (bs, 1H); 8.32-8.20 (m, 1H); 7.51 (d, J=8.1 Hz, 1H); 5.13 (s, 2H).

A solution of pentafluorophenyl 7-fluoro-1-hydroxy-1,3-dihydrobenzo[c][1,2]oxaborole-6-carboxylate (10, 9.46 g, 26.1 mmol), (2-aminoethyl)glycine (11, 1.54 g, 13.1 mmol) and triethylamine (14.5 mL, 105 mmol) in N,N-dimethylformamide (200 mL) was stirred at ambient temperature overnight (16 hours). The reaction mixture was evaporated and it was tried to dissolve it in dichloromethane to do the TLC. It was discovered that the crude product is insoluble in dichloromethane, ethyl acetate and acetonitrile. Therefore, it was precipitated from ethyl acetate (0.5 L) and the solid was collected by centrifugation. The first precipitate (A) was washed with 0.5 M solution of hydrochloride (2×50 mL) to give the second precipitate (B) that was filtered off and kept. The filtrate was freeze-dried to give product 12 contaminated with salts. The salts were removed by dissolving in tetrahydrofuran and filtering. Remaining solution was evaporated in vacuo to give the first crop of product 12. The precipitate (B) was dissolved in acetonitrile and water (3:1), filtered and the remaining solution was freeze dried. The resulting solid was dissolved in tetrahydrofuran, the precipitated salts were filtered off and the filtrate was evaporated in vacuo to give the second part of N-(7-fluoro-1-hydroxy-1,3-dihydrobenzo[c][1,2]oxaborole-6-carbonyl)-N-(2-(7-fluoro-1-hydroxy-1,3-dihydrobenzo[c][1,2]oxaborole-6-carboxamido)ethyl)glycine (12) as a beige solid. Yield: 2.09 g (34%). ¹H NMR spectrum (300 MHz, DMSO-d6, δH): 9.41-9.33 (m, 2H); 8.42-8.18 (m, 1H); 7.81-7.64 (m, 1H); 7.43-7.11 (m, 3H); 5.07-4.97 (m, 4H); 4.22 (s, 1H); 3.98 (s, 1H); 3.68 (t, J=6.5 Hz, 1H); 3.60-3.40 (m, 3H). LC-MS: 475.5 (M+H)+.

### Example 33: 2,5-Dioxopyrrolidin-1-yl 2-((oxobis(3-(4,4,5,5-tetramethyl-1,3,2-dioxaborolan-2-yl)-5-trifluoromethyl)phenyl)-λ⁶-sulfaylidene)amino)acetate

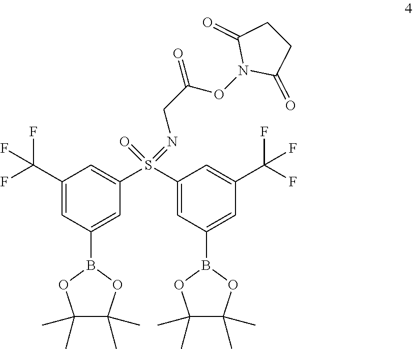 [SMILES: CC1(C)OB(c2cc(C(F)(F)F)cc(S(=O)(=NCC(=O)ON3C(=O)CCC3=O)c3cc(B4OC(C)(C)C(C)(C)O4)cc(C(F)(F)F)c3)c2)OC1(C)C]

tert-Butyl 2-((oxobis(3-(trifluoromethyl)phenyl)-λ⁶-sulfanylidene)amino)acetate (1, 2.05 g, 4.38 mmol), bis(pinacolato)diboron (2.78 g, 11.0 mmol), (1,5-cyclooctadiene)(methoxy)iridium(I) dimer (87.0 mg, 0.13 mmol) and 4,4-di-tert-butyl-2,2-dipyridyl (dtbpy, 82.0 mg, 0.31 mmol) were dissolved in degassed tetrahydrofuran (12 mL) under argon. The resulting mixture was warmed to 60° C. and heated at this temperature overnight. The mixture was evaporated to dryness; and the residue purified by flash column chromatography (Silicagel 60, 0.040-0.063 mm; eluent:dichloromethane/ethyl acetate 10:0 to 4:1) to give tert-butyl 2-((oxobis(3-(4,4,5,5-tetramethyl-1,3,2-dioxaborolan-2-yl)-5-(trifluoromethyl)phenyl)-λ⁶-sulfanylidene)amino)acetate (2) as off-white foam.

Yield: 2.92 g (93%). ¹H NMR spectrum (300 MHz, CDCl₃, δH): 8.59 (s, 2H); 8.42 (s, 2H); 8.21 (s, 2H); 3.76 (s, 2H); 1.51 (s, 9H); 1.36 (s, 12H); 1.35 (s, 12H).

¹⁹F NMR spectrum (282 MHz, CDCl₃, δF): −62.55 (s). LC-MS: 556.6 (M−2×pinacol+H)+, 638.8 (M−pinacol+H)+, 721.0 (M+H)+.

Trifluoroacetic acid (24 mL) was added to a solution of tert-butyl 2-((oxobis(3-(4,4,5,5-tetramethyl-1,3,2-dioxaborolan-2-yl)-5-(trifluoromethyl)phenyl)-λ⁶-sulfanylidene)amino)acetate (2, 2.91 g, 4.05 mmol) in dichloromethane (8 mL) and the mixture was stirred for 2 hours at room temperature. The mixture was evaporated to dryness in vacuo, and the residue was evaporated from toluene (3×20 mL) and dichloromethane (3×20 mL). The residue was partitioned between dichloromethane (200 mL) and 0.5 M aqueous solution of sodium hydroxide (250 mL). Separated aqueous phase was washed with dichloromethane (2×100 mL), acidified with 1 M hydrochloric acid (200 mL) and extracted with ethyl acetate (3×250 mL). Combined ethyl acetate extracts were dried over anhydrous sodium sulfate and evaporated in vacuo to give 2-((oxobis(3-(4,4,5,5-tetramethyl-1,3,2-dioxaborolan-2-yl)-5-(trifluoromethyl)phenyl)-λ⁶-sulfanylidene)amino)acetic acid (3) as off-white foam. Yield: 2.30 g (86%). ¹H NMR spectrum (300 MHz, CDCl₃, δH): 8.55 (s, 2H); 8.33 (s, 2H); 8.29 (s, 2H); 3.85 (s, 2H); 1.39 (s, 24H). ¹⁹F NMR spectrum (282 MHz, CDCl₃, δF): −62.69 (s). LC-MS: 500.5 (M−2×pinacol+H)+, 582.6 (M−pinacol+H)+, 664.8 (M+H)+.

Dry acetonitrile (16.2 mL) was added to 2-((oxobis(3-(4,4,5,5-tetramethyl-1,3,2-dioxaborolan-2-yl)-5-(trifluoromethyl)phenyl)-λ⁶-sulfanylidene)amino)acetic acid (3, 2.15 g, 3.24 mmol) and N,N-disuccinimidyl carbonate (DSC, 1.25 g, 4.86 mmol) under argon. Pyridine (392 mL, 4.86 mmol) was added and the mixture was sonicated to form a fine suspension. The resulting suspension was stirred for 4 hours to give a clear solution. Additional amount of N,N-disuccinimidyl carbonate (DSC, 415 mg, 1.62 mmol) and pyridine (131 mL, 1.62 mmol) was added, and the mixture was stirred at room temperature overnight. LC/MS analysis showed a complete conversion to activated ester. The mixture was evaporated to dryness and the residue was partitioned between ethyl acetate (200 mL) and 0.1 M aqueous solution of hydrochloric acid (100 mL). The phases were separated, the organic one was washed with 0.1 M aqueous solution of hydrochloric acid (2×50 mL) and brine (50 mL), dried over anhydrous sodium sulfate and evaporated to dryness. The residue was dissolved in dichloromethane (40 mL), followed by addition of pinacol (383 mg, 3.24 mmol). The solution was evaporated and the residue was evaporated from dichloromethane (3×40 mL). The resulting foam was washed with cyclohexane (2×50 mL), re-dissolved in dichloromethane (40 mL), evaporated and dried in vacuo to give the title compound (4) as off-white foam.

Yield: 1.82 g (74%). ¹H NMR spectrum (300 MHz, CDCl₃, δH): 8.57 (s, 2H); 8.37 (s, 2H); 8.24 (s, 2H); 4.20 (s, 2H); 2.83 (s, 4H); 1.36 (s, 24H). ¹⁹F NMR spectrum (282 MHz, CDCl₃, δF): −62.66 (s). LC-MS: 761.9 (M+H)+.

### Example 34: 2,5-Dioxopyrrolidin-1-yl 3-(4,4,5,5-tetramethyl-1,3,2-dioxaborolan-2-yl)-5-((3-(4,4,5,5-tetramethyl-1,3,2-dioxaborolan-2-yl)-5-(trifluoromethyl)phenyl)sulfonyl)benzoate

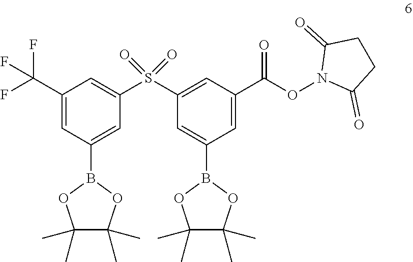 [SMILES: CC1(C)OB(c2cc(C(=O)ON3C(=O)CCC3=O)cc(S(=O)(=O)c3cc(B4OC(C)(C)C(C)(C)O4)cc(C(F)(F)F)c3)c2)OC1(C)C]

Methyl 3-iodobenzoate (2, 10.5 g, 40.0 mmol), anhydrous potassium carbonate (11.0 g, 80.0 mmol), copper iodide (1.52 g, 8.00 mmol) and 3-trifluormethylbenzenethiol (1, 8.22 mL, 60.0 mmol) were suspended in dry 1,2-dimethoxyethane (100 mL) and the resulting suspension was stirred for 48 hours at 80° C. After cooling to ambient temperature, the reaction mixture was diluted with cyclohexane (300 mL), filtered through a pad of silicagel (125 g) topped with celite (washed with ethyl acetate/cyclohexane 1:10, 3×200 mL) and evaporated in vacuo. The residue was dissolved in acetic acid (120 mL) and 30% aqueous solution of hydrogen peroxide (16.0 mL, 156 mmol) was added in portions (heat evolution). After stirring for 16 hours at 80° C. (oil bath), the reaction mixture was evaporated in vacuo, taken up in ethyl acetate (400 mL) and washed with water (400 mL) and brine (400 mL). Drying of the organic layer with anhydrous sodium sulfate, filtration and evaporation in vacuo gave the methyl ester 4 as yellow oil, which was subjected to flash column chromatography (Silicagel 300, 0.063-0.200 mm; eluent:cyclohexane/ethyl acetate 4:1) to give methyl 3-((3-(trifluoromethyl)phenyl)sulfonyl)benzoate (4) as colorless oil. Yield: 5.40 g (39%). LC-MS: 346.0 (M+H)+.

Methyl 3-((3-(trifluoromethyl)phenyl)sulfonyl)benzoate (4, 5.40 g, 15.7 mmol), bis(pinacolato)diboron (9.97 g, 39.0 mmol), (1,5-cyclooctadiene)(methoxy)iridium(I) dimer (310 mg, 0.47 mmol) and 4,4-di-tert-butyl-2,2-dipyridyl (dtbpy, 295 mg, 1.10 mmol) were dissolved in dry, degassed tetrahydrofuran (30 mL) under nitrogen. The reaction mixture was stirred at 50° C. (oil bath) for 16 hours. After cooling to ambient temperature, ice-cold water (30 mL) was added slowly to decompose generated pinacolborane (hydrogen gas evolution). After 30 minutes, lithium hydroxide monohydrate (6.59 g, 157 mmol) was added and the resulting mixture was stirred for three hours at ambient temperature before it was taken up in water (300 mL) and extracted with dichloromethane (3×60 mL). Dichloromethane extracts were discarded and the aqueous layer was acidified to pH 2 by concentrated hydrochloric acid. Aqueous layer was extracted with ethyl acetate (50 mL) and discarded. Organic layer was washed with brine (3×50 mL), dried over anhydrous sodium sulfate, filtered and concentrated in vacuo. The resulting yellowish foam was treated with pinacol (118 mg, 1.00 mmol) and dissolved in warm acetonitrile (20 mL). The solution was left for crystallization overnight in the freezer. The precipitated product was collected by filtration, washed with chilled acetonitrile and dried in air top give 3-(4,4,5,5-tetramethyl-1,3,2-dioxaborolan-2-yl)-5-((3-(4,4,5,5-tetramethyl-1,3,2-dioxaborolan-2-yl)-5-(trifluoromethyl)phenyl)sulfonyl)benzoic acid (5) as colorless solid. Yield: 5.90 g (65%). LC-MS: 582.6 (M+H)+.

3-(4,4,5,5-Tetramethyl-1,3,2-dioxaborolan-2-yl)-5-((3-(4,4,5,5-tetramethyl-1,3,2-dioxaborolan-2-yl)-5-(trifluoromethyl)phenyl)sulfonyl)benzoic acid (5, 5.90 g, 10.1 mmol) and bis(succinimidyl)carbonate (3.63 g, 14.2 mmol) were suspended in anhydrous acetonitrile (45 mL) under nitrogen and pyridine (1.14 mL, 14.2 mmol). The reaction mixture was heated to effect dissolution. After stirring for 16 hours, the reaction mixture was concentrated in vacuo and the residue was taken up in ethyl acetate (200 mL) and washed with brine (3×200 mL). The organic layer was dried over anhydrous sodium sulfate, filtered and concentrated under reduced pressure to give an off-white solid. Pinacol (473 mg, 4.00 mmol) was added and mixture was left to stir for 1 hour in acetonitrile (30 mL). Acetonitrile was evaporated in vacuo. Resulting white foam was dissolved in hexane (30 mL) and the solution was left for crystallization overnight at ambient temperature to give 2,5-dioxopyrrolidin-1-yl 3-(4,4,5,5-tetramethyl-1,3,2-dioxaborolan-2-yl)-5-((3-(4,4,5,5-tetramethyl-1,3,2-dioxaborolan-2-yl)-5-(trifluoromethyl)phenyl)sulfonyl)benzoate (6) as white solid. Yield: 6.50 g (94%).

¹H NMR spectrum (300 MHz, CDCl₃, δH): 8.76-8.72 (m, 2H); 8.67 (s, 1H); 8.55 (s, 1H); 8.32 (s, 1H); 8.27 (s, 1H); 2.92 (s, 4H); 1.37 (s, 12H) overlapping with 1.37 (s, 12H). ¹⁹F NMR spectrum (300 MHz, CDCl₃, δF): 62.64 (s, 3H). LC-MS: 680.6 (M−H)+.

### Example 35: N-(1-Hydroxy-5-(trifluoromethyl)-1,3-dihydrobenzo[c][1,2]oxaborole-6-carbonyl)-N-(2-(1-hydroxy-5-(trifluoromethyl)-1,3-dihydrobenzo[c][1,2]oxaborole-6-carboxamido)ethyl)glycine

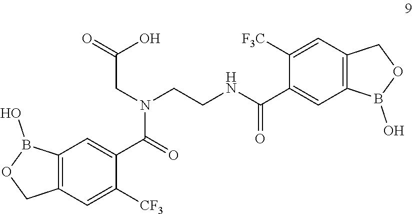 [SMILES: O=C(O)CN(CCNC(=O)c1cc2c(cc1C(F)(F)F)COB2O)C(=O)c1cc2c(cc1C(F)(F)F)COB2O]

4-Methyl-2-(trifluoromethyl)benzoic acid (1, 25.0 g, 123 mmol) was dissolved in sulfuric acid (183 mL) followed by addition of N-iodosuccinimide (33.1 g, 147 mmol). The resulting mixture was stirred overnight at room temperature then it was poured onto ice. When ice was completely melted the mixture was extracted with ethyl acetate (500 mL). Organic layer was washed with 5% aqueous solution of sodium thiosulfate (2×250 mL) and water (1×250 mL), dried over anhydrous sodium sulfate, filtered and evaporated to dryness affording 5-iodo-4-methyl-2-(trifluoromethyl)benzoic acid (2) as beige powder. Yield: 37.7 g (93%).

¹H NMR spectrum (300 MHz, DMSO-d6, δH): 13.68 (bs, 1H); 8.22 (s, 1H); 7.76 (s, 1H); 2.47 (s, 3H).

Mixture of 5-iodo-4-methyl-2-(trifluoromethyl)benzoic acid (2, 22.2 g, 67.2 mmol), trimethyl orthoformate (14.7 mL, 134 mmol) and methanesulfonic acid (2.8 mL) in methanol (135 mL) was refluxed at 80° C. under nitrogen atmosphere overnight. Solvent was evaporated. The residue was dissolved in 5% aqueous solution of sodium carbonate (200 mL) and extracted with ethyl acetate (3×250 mL). Combined organic layers were washed with water (1×300 mL) and brine (1×200 mL), dried over anhydrous sodium sulfate, filtered and evaporated. The residue was purified by quick flash column chromatography (Silicagel 60, 0.040-0.063 mm; eluent:cyclohexane/ethyl acetate 9:1) to give methyl 5-iodo-4-methyl-2-(trifluoromethyl)benzoate (3) as white crystals. Yield: 35.9 g (91%). RF (cyclohexane/ethyl acetate 9:1): 0.50. ¹H NMR spectrum (300 MHz, CDCl₃, δH): 8.26 (s, 1H); 7.57 (s, 1H); 3.93 (s, 3H); 2.53 (s, 3H).

A mixture of methyl 5-iodo-4-methyl-2-(trifluoromethyl)benzoate (3, 35.9 g, 104 mmol), N-bromosuccinimide (20.4 g, 114 mmol) and 2,2-azobis(2-methylpropionitrile) (AIBN, 5.12 g, 31.2 mmol) in benzotrifluoride (95 mL) was stirred at 85 C overnight. Full conversion was not achieved but the reaction was worked up. Dichloromethane (150 mL) was added and the mixture was washed with water (3×100 mL). Organic layer was dried over anhydrous sodium sulfate, filtered and evaporated. The residue was dissolved in acetonitrile (440 mL) and potassium acetate (10.2 g, 104 mmol) was added. The mixture was stirred at 75 C overnight. The insoluble material was filtered off and the filtrate was evaporated. The residue was purified by flash column chromatography (Silicagel 60, 0.040-0.063 mm; eluent: cyclohexane/dichloromethane 4:1 to 1:1.5) to give methyl 4-(acetoxymethyl)-5-iodo-2-(trifluoromethyl)benzoate (4) as white powder. Yield: 17.5 g (42%). RF (cyclohexane/ethyl acetate 9:1): 0.35. ¹H NMR spectrum (300 MHz, CDCl₃, δH): 8.28 (s, 1H); 7.70 (s, 1H); 5.16 (s, 2H); 3.95 (s, 3H); 2.20 (s, 3H). ¹⁹F NMR spectrum (282 MHz, CDCl₃, δF): −59.96 (s).

A mixture of methyl 4-(acetoxymethyl)-5-iodo-2-(trifluoromethyl)benzoate (4, 17.5 g, 43.5 mmol), bis(pinacolato)diboron (14.3 g, 56.5 mmol) and dry potassium acetate (21.3 g, 217 mmol) in dry N,N-dimethylsulfoxide (110 mL) was degassed; then [1,1-bis(diphenylphosphino)ferrocene]dichloropalladium (1.59 g, 2.17 mmol) was added. Reaction mixture was stirred under nitrogen atmosphere at 95° C. overnight. After cooling down diethyl ether (500 mL) was added and the precipitate was filtered off through celite pad. The filtrate was washed with 5% aqueous solution of sodium chloride (3×500 mL). Organic layer was dried over anhydrous sodium sulfate, filtered and evaporated affording methyl 4-(acetoxymethyl)-5-(4,4,5,5-tetramethyl-1,3,2-dioxaborolan-2-yl)-2-(trifluoromethyl)benzoate (5) as black oil. This oil was used in the next step without further purification.

Yield: 22.5 g. ¹H NMR spectrum (300 MHz, CDCl₃, δH): 8.22 (s, 1H); 7.74 (s, 1H); 5.44 (s, 2H); 3.94 (s, 3H); 2.14 (s, 3H); 1.36 (s, 12H). ¹⁹F NMR spectrum (282 MHz, CDCl₃, δF): −60.07 (s).

Methyl 4-(acetoxymethyl)-5-(4,4,5,5-tetramethyl-1,3,2-dioxaborolan-2-yl)-2-(trifluoromethyl)benzoate (5, 17.5 g, 43.5 mmol) was suspended in a solution of sodium hydroxide (8.70 g, 217 mmol) in water (150 mL). The mixture was stirred for 6 hours at room temperature then it was extracted with diethyl ether (2×200 mL). Aqueous phase was acidified with concentrated hydrochloric acid (18.9 mL) and resulting mixture was stirred overnight at room temperature. The precipitate was filtered, washed with water and dried to give 1-hydroxy-5-(trifluoromethyl)-1,3-dihydrobenzo[c][1,2]oxaborole-6-carboxylic acid (6) as grey powder. Yield: 7.62 g (71%). ¹H NMR spectrum (300 MHz, DMSO-d6, δH): 13.50 (bs, 1H); 9.57 (s, 1H); 8.16 (s, 1H); 7.92 (s, 1H); 5.11 (s, 2H). ¹⁹F NMR spectrum (282 MHz, DMSO-d6, δF): −57.91 (s). LC-MS: 245.9 (M−H)−.

1-Hydroxy-5-(trifluoromethyl)-1,3-dihydrobenzo[c][1,2]oxaborole-6-carboxylic acid (6, 6.71 g, 27.3 mmol) was dissolved in tetrahydrofuran/dichloromethane mixture (1:1, 50 mL) followed by addition of 2,3,4,5,6-pentrafluorophenol (5.03 g, 27.3 mmol) and N-(3-dimethylaminopropyl)-N-ethylcarbodiimide hydrochloride (5.23 g, 27.3 mmol). The mixture was stirred overnight at room temperature. Solvent was evaporated. The residue was dissolved in ethyl acetate (150 mL) and washed with water (3×100 mL) and brine (1×100 mL). Organic layer was dried over anhydrous sodium sulfate, filtered and evaporated. The residue was dissolved in diethyl ether (10 mL) and n-hexane (200 mL) was added. The precipitate was filtered off and the filtrate was evaporated. The same procedure was repeated with the precipitate twice. All the filtrates were combined together and evaporated to dryness to afford pentafluorophenyl 1-hydroxy-5-(trifluoromethyl)-1,3-dihydrobenzo[c][1,2]oxaborole-6-carboxylate (7) as yellow tough oil. Yield: 9.76 g (87%).

¹H NMR spectrum (300 MHz, DMSO-d6, δH): 9.74 (s, 1H); 8.53 (s, 1H); 8.16 (s, 1H); 5.18 (s, 2H).

Pentafluorophenyl 1-hydroxy-5-(trifluoromethyl)-1,3-dihydrobenzo[c][1,2]oxaborole-6-carboxylate (7, 9.51 g, 23.1 mmol) was dissolved in N,N-dimethylformamide (30 mL). Subsequently N,N-diisopropylethylamine (10.1 mL, 57.7 mmol) and a solution of (2-aminoethyl)glycine hydrochloride (8, 1.78 g, 11.5 mmol) in water (30 mL) were added. Resulting mixture was stirred overnight at room temperature. Then the solvents were evaporated. The residue was dissolved in ethyl acetate (200 mL) and washed 1 M aqueous solution of hydrochloric acid (1×200 mL), water (2×200 mL) and brine (1×150 mL). Organic layer was dried over anhydrous sodium sulfate, filtered and evaporated. The residue was treated with cyclohexane. The precipitate was filtered, washed with cyclohexane and purified by flash column chromatography (Silicagel 60, 0.040-0.063 mm; eluent: dichloromethane/methanol/formic acid 10:1:0.05). Fractions containing product were combined and evaporated. The residue was treated with cyclohexane. The precipitate was filtered, washed with cyclohexane, dissolved in acetonitrile (50 mL) and freeze-dried to give the title compound (9) as beige powder. Yield: 3.63 g (55%).

¹H NMR spectrum (300 MHz, AcOD-d4, 80 C, δH): 8.04-7.66 (m, 4H); 5.28-5.04 (m, 4H); 4.63-4.34 (m, 1H); 4.22-3.78 (m, 3H); 3.72-3.49 (m, 2H). LC-MS: 574.0 (M+H)+.

### Example 36: N-(4-Chloro-1-hydroxy-1,3-dihydrobenzo[c][1,2]oxaborole-6-carbonyl)-N-(2-(4-chloro-1-hydroxy-1,3-dihydrobenzo[c][1,2]oxaborole-6-carboxamido)ethyl)glycine

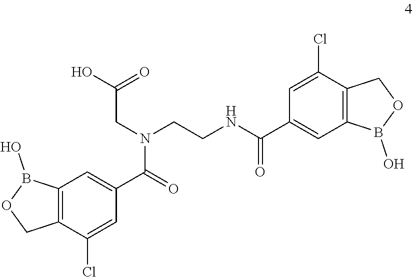 [SMILES: O=C(O)CN(CCNC(=O)c1cc(Cl)c2c(c1)B(O)OC2)C(=O)c1cc(Cl)c2c(c1)B(O)OC2]

N-(3-Dimethylaminopropyl)-N-ethylcarbodiimide hydrochloride (EDC.HCl, 6.20 g, 23.1 mmol) was added to a suspension of 4-chloro-1-hydroxy-1,3-dihydrobenzo[c][1,2]oxaborole-6-carboxylic acid (1, 4.90 g, 23.1 mmol) and pentafluorophenol (Pfp-OH, 5.53 g, 23.1 mmol) in dichloromethane (70 mL) and the mixture was stirred at room temperature overnight. Solvent was evaporated to dryness. Residue was partionated between ethyl acetate (200 mL) and 10% aqueous solution of potassium hydrogensulfate (200 mL). Organic layer was separated and washed with water (2×100 mL), dried over anhydrous sodium sulfate and evaporated in vacuo. Residue was dissolved in dichloromethane and placed in the fridge overnight. The solid was filtered off and washed with ethyl acetate (2×20 mL). The filtrates were combined and evaporated to dryness. Cyclohexane (100 mL) was added to the residue and the mixture was stirred at room temperature for 15 minutes. The mixture was decanted and the sediment was dried in vacuo to give pentafluorophenyl 4-chloro-1-hydroxy-1,3-dihydrobenzo[c][1,2]oxaborole-6-carboxylate (2) as off-white solid. Yield: 8.29 g (95%). ¹H NMR spectrum (300 MHz, DMSO-d6, δH): 9.83 (bs, 1H); 8.61 (s, 1H); 8.26 (s, 1H); 5.13 (s, 2H). LC-MS: 377.4 (M−H)−.

Triethylamine (10.0 mL, 131.6 mmol) was added to a mixture of pentafluorophenyl 1-hydroxy-1,3-dihydrobenzo[c][1,2]oxaborole-6-carboxylate (2, 8.29 g, 21.9 mmol) and N-2-aminoethylglycine (3, 1.30 g, 1.70 mmol) in solution N,N-dimethylformamide/water (2:1, 60 mL) and the resulting solution was stirred at room temperature overnight. Afterwards, it was acidified with 1 M aqueous solution of potassium bisulfate (200 mL) and extracted with ethyl acetate (3×250 mL). The organic layer was dried over anhydrous sodium sulfate, filtered and evaporated. The residue was co-distilled with toluene (3×100 mL) and triturated with diethyl ether (60 mL). The precipitate was filtered, washed with diethyl ether (2×50 mL) and air dried. The obtained powder was dissolved in acetonitrile/water mixture (2:1, 20 mL) and freeze-dried to give compound 4 as colorless solid. Yield: 1.50 g (15%). ¹H NMR spectrum (300 MHz, DMSO-d6, δH): 12.87 (bs, 1H); 9.59-9.41 (m, 2H); 8.77-8.54 (m, 5H); 5.07-4.88 (m, 4H); 4.25-3.92 (m, 2H); 3.60-3.24 (m, 4H). LC-MS: 507.3 (M+H)+.

### Example 37: (S)-4-((2S)-2,3-Bis(1-hydroxy-4-(trifluoromethyl)-1,3-dihydrobenzo[c][1,2]oxaborole-6-carboxamido)propanamido)-5-(tert-butoxy)-5-oxopentanoic acid

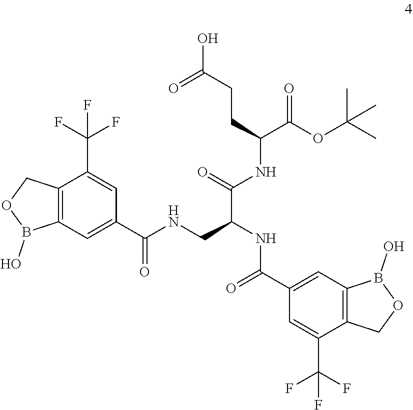 [SMILES: CC(C)(C)OC(=O)[C@H](CCC(=O)O)NC(=O)[C@H](CNC(=O)c1cc2c(c(C(F)(F)F)c1)COB2O)NC(=O)c1cc2c(c(C(F)(F)F)c1)COB2O]

Solution of pentafluorophenol (35.1 g, 191 mmol), 1-hydroxy-4-(trifluoromethyl)-1,3-dihydrobenzo[c][1,2]oxaborole-6-carboxylic acid (1, 40.8 g, 166 mmol) and N,N′-dicyclohexylcarbodiimide (DCC, 39.3 g, 191 mmol) in acetonitrile (1 L) was stirred at ambient temperature for 24 hours. The reaction mixture was filtered, evaporated, dissolved in acetonitrile, re-filtered and evaporated. The crude product was precipitated in dichloromethane (1 L) and filtered to give the pentafluorophenyl 1-hydroxy-4-(trifluoromethyl)-1,3-dihydrobenzo[c][1,2]oxaborole-6-carboxylate (2) as white solid. Yield: 52.8 g (77%). ¹H NMR spectrum (300 MHz, DMSO-d6, δH): 9.79 (s, 1H); 8.86 (s, 1H); 8.46 (s, 1H); 5.30 (s, 2H).

2-Chlorotrityl chloride resin 100-200 mesh 1.5 mmol/g (3, 4.47 g, 6.71 mmol) was left to swell in dry dichloromethane (30 mL) for 30 minutes. A solution of (2S)-5-(tert-butoxy)-2-{[(9H-fluoren-9-ylmethoxy)carbonyl]amino}-5-oxopentanoic acid (Fmoc-Glu-OtBu, 1.90 g, 4.47 mmol) and N,N-diisopropylethylamine (2.96 mL, 17.0 mmol) in dry dichloromethane (30 mL) was added to resin and the mixture was shaken overnight. Resin was filtered and treated with a solution of N,N-diisopropylethylamine (1.56 mL, 8.95 mmol) in methanol/dichloromethane mixture (4:1, 2×5 min, 2×40 mL). Then resin was washed with N,N-dimethylformamide (2×30 mL), dichloromethane (2×40 mL) and N,N-dimethylformamide (3×40 mL). Fmoc group was removed by treatment with 20% piperidine in N,N-dimethylformamide (1×5 min, 1×20 min, 2×40 mL). Resin was washed with N,N-dimethylformamide (3×40 mL), 2-propanol (2×40 mL) and dichloromethane (3×40 mL). Solution of (2S)-2,3-bis((((9H-fluoren-9-yl)methoxy)carbonyl)amino)propanoic acid (Fmoc-Dap(Fmoc)-OH, 3.68 g, 6.71 mmol), 1-[bis(dimethylamino)methylene]-1H-1,2,3-triazolo[4,5-b]pyridinium 3-oxid hexafluorophosphate (HATU, 2.55 g, 6.71 mmol) and 2,4,6-trimethylpyridine (1.60 mL, 12.1 mmol) in N,N-dimethylformamide (40 mL) was added to resin and mixture was shaken for 2 hours. Resin was filtered and washed with N,N-dimethylformamide (2×40 mL), dichloromethane (2×40 mL) and N,N-dimethylformamide (2×40 mL). Fmoc groups were removed by treatment with 20% piperidine in N,N-dimethylformamide (1×5 min, 1×30 min, 2×40 mL). Resin was washed with N,N-dimethylformamide (3×40 mL), 2-propanol (2×40 mL) and dichloromethane (3×40 mL). Solution of pentaflurophenyl 1-hydroxy-4-(trifluoromethyl)-1,3-dihydrobenzo[c][1,2]oxaborole-6-carboxylate (2, 5.53 g, 13.4 mmol) and triethylamine (4.99 mL, 35.8 mmol) in N,N-dimethylformamide (40 mL) was added to resin and mixture was shaken overnight. Resin was filtered and washed with N,N-dimethylformamide (6×40 mL) and dichloromethane (10×50 mL). The product was cleaved from resin by treatment with 2,2,2-trifluoroethanol (60 mL) for 16 hours. Resin was filtered off and washed with dichloromethane (4×50 mL). Crude product (4) was dried in vacuo and extracted with ethyl acetate (2×70 mL) and 1 M aqueous solution of potassium hydrogen sulfate (50 mL), organic phases were dried over anhydrous sodium sulfate, filtered and the solvent was evaporated. Crude product was then triturated in diethyl ether (20 mL) to give (S)-4-((2S)-2,3-bis(1-hydroxy-4-(trifluoromethyl)-1,3-dihydrobenzo[c][1,2]oxaborole-6-carboxamido)propanamido)-5-(tert-butoxy)-5-oxopentanoic acid (4) as beige solid. Yield: 1.89 g (57%). ¹H NMR spectrum (300 MHz, AcOD-d4, δH): 8.50 (s, 1H); 8.46 (s, 1H); 8.29 (s, 1H); 8.26 (s, 1H); 5.28 (d, J=2.6 Hz, 4H); 5.20 (t, J=5.9 Hz, 1H); 4.55 (dd, J=8.5 and 5.2 Hz, 1H); 4.08 (dd, J=6.0 and 2.1 Hz, 2H); 2.57-2.42 (m, 2H); 2.34-2.16 (m, 1H); 2.17-2.08 (m, 1H); 1.47 (s, 9H). LC-MS: 746.3 (M+H)+.

### Example 38: N-(4-(Difluoromethyl)-1-hydroxy-1,3-dihydrobenzo[c][1,2]oxaborole-6-carbonyl)-N-(2-(4-(difluoromethyl)-1-hydroxy-1,3-dihydrobenzo[c][1,2]oxaborole-6-carboxamido)ethyl)glycine

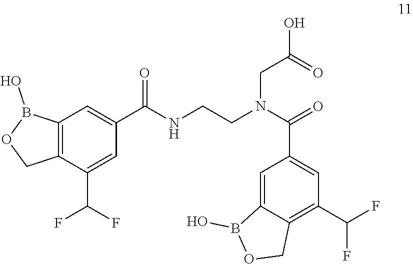 [SMILES: O=C(O)CN(CCNC(=O)c1cc2c(c(C(F)F)c1)COB2O)C(=O)c1cc2c(c(C(F)F)c1)COB2O]

Concentrated sulfuric acid (35 mL) was added to a solution of 3-bromo-5-iodo-4-methylbenzoic acid (1, 55.4 g, 162 mmol) in methanol (1.2 L) and the reaction mixture was allowed to stir under reflux overnight. The reaction mixture was then evaporated under reduced pressure, dissolved in diethyl ether (700 mL), washed with water (2×300 mL) and saturated solution of potassium carbonate (1×300 mL). Organic layer was separated, dried over anhydrous sodium sulfate, filtered and evaporated to provide methyl 3-bromo-5-iodo-4-methylbenzoate (2) as white solid. Yield: 50.0 g (87%). ¹H NMR spectrum (300 MHz, DMSO-d6, δH): 8.32 (d, J=1.7 Hz, 1H); 8.09 (d, J=1.3 Hz, 1H); 3.86 (s, 3H); 2.65 (s, 3H).

To a solution of methyl 3-bromo-5-iodo-4-methylbenzoate (2, 37.3 g, 105 mmol) in dry tetrahydrofuran (250 mL) 1.3 M solution of isopropylmagnesium chloride lithium chloride complex in tetrahydrofuran (89.0 mL, 115 mmol) was added dropwise at −30 C under inert atmosphere and was stirred for 20 minutes. Then N,N-dimethylformamide (12.2 mL, 158 mmol) was added at −30 C. The reaction mixture was allowed to warm to ambient temperature and stirred for 16 hours. The reaction mixture was then evaporated under reduced pressure, dissolved in ethyl acetate (300 mL) and washed with water (2×200 mL). Organic layer was separated, dried over anhydrous sodium sulfate, filtered and evaporated to provide methyl 3-bromo-5-formyl-4-methylbenzoate (3) as white solid. Yield: 24.9 g (92%).

¹H NMR spectrum (300 MHz, CDCl₃, δH): 10.27 (s, 1H); 8.53-8.34 (m, 2H); 3.97 (s, 3H); 2.82 (s, 3H).

Solution of methyl 3-bromo-5-formyl-4-methylbenzoate (3, 24.8 g, 96.5 mmol) and (diethylamino)sulfur trifluoride (DAST, 25.5 mL, 193 mmol) in dichloromethane (300 mL) was stirred at ambient temperature for 16 hours. Reaction was quenched by addition of water (200 mL) and extracted with dichloromethane (2×200 mL). Organic layers were combined, dried over anhydrous sodium sulfate, filtered and evaporated to provide methyl 3-bromo-5-(difluoromethyl)-4-methylbenzoate (4) as white solid. Yield: 23.3 g (87%). ¹H NMR spectrum (300 MHz, CDCl₃, δH): 8.35 (d, J=1.1 Hz, 1H); 8.14 (d, J=0.9 Hz, 1H); 6.78 (t, J=54.8 Hz, 1H); 3.93 (s, 3H); 2.55 (t, J=1.4 Hz, 3H).

Solution of N-bromosuccinimide (16.4 g, 91.9 mmol), methyl 3-bromo-5-(difluoromethyl)-4-methylbenzoate (4, 23.3 g, 83.5 mmol) and 2,2-azobis(2-methylpropionitrile) (AIBN, 1.36 g, 8.36 mmol) in α,α,α-trifluorotoluene (120 mL) was stirred overnight at 85 C. Reaction mixture was evaporated and then extracted with diethyl ether (2×300 mL). Organic layers were washed with brine (1×150 mL). Organic layer was separated, dried over anhydrous sodium sulfate, filtered and evaporated giving crude methyl 3-bromo-4-(bromomethyl)-5-(difluoromethyl)benzoate (5) which was stirred with potassium acetate (16.4 g, 167 mmol) in acetonitrile (300 mL) at 75 C overnight. The suspension was filtered through a short pad of celite and evaporated. The crude product was dissolved in dichloromethane and filtered again. The filtrate was evaporated and purified by column chromatography (Silicagel 60, 0.063-0.200 mm; eluent:cyclohexane/ethyl acetate 9:1) to give methyl 4-(acetoxymethyl)-3-bromo-5-(difluoromethyl)benzoate (6) as white solid. Yield: 17.1 g (61%). RF (SiO2, hexane/ethyl acetate 9:1): 0.50. ¹H NMR spectrum (300 MHz, CDCl₃, δH): 8.40 (s, 1H); 8.26 (s, 1H); 7.02 (t, J=54.7 Hz, 1H); 5.38 (s, 2H); 3.97 (s, 3H); 2.11 (s, 3H).

Solution of methyl 4-(acetoxymethyl)-3-bromo-5-(difluoromethyl)benzoate (6, 17.1 g, 50.7 mmol), bis(pinacolato)diboron (14.2 g, 55.7 mmol), potassium acetate (14.9 g, 152 mmol) and [1,1-bis(diphenylphosphino)ferrocene]dichloropalladium(II) (1.24 g, 1.52 mmol) in dry dioxane (200 mL) was allowed to stir at 75° C. under argon atmosphere for 2 days. Then the reaction mixture was cooled to ambient temperature, filtered and evaporated. The crude product was filtered through silica gel column (Silicagel, 0.063-0.200 mm; eluent: cyclohexane/ethyl acetate 9:1) to provide methyl 4-(acetoxymethyl)-3-(difluoromethyl)-5-(4,4,5,5-tetramethyl-1,3,2-dioxaborolan-2-yl)benzoate (7). Yield: 16.3 g (84%). RF (SiO2, cyclohexane/ethyl acetate 9:1): 0.30. ¹H NMR spectrum (300 MHz, CDCl₃, δH): 8.57 (s, 1H); 8.38 (s, 1H); 7.04 (t, J=55.1 Hz, 1H); 5.54 (s, 2H); 3.97 (s, 3H); 2.06 (s, 3H); 1.39 (s, 12H).

Solution of methyl 4-(acetoxymethyl)-3-(difluoromethyl)-5-(4,4,5,5-tetramethyl-1,3,2-dioxaborolan-2-yl)benzoate (7, 16.3 g, 42.3 mmol) and sodium hydroxide (8.45 g, 212 mmol) in water (200 mL) was stirred at ambient temperature for 3 hours. Then solution of concentrated hydrochloric acid (20 mL) in water (50 mL) was added to lower the pH to 1. The reaction mixture was left in the fridge overnight. Precipitate was filtered and dried to provide 4-(difluoromethyl)-1-hydroxy-1,3-dihydrobenzo[c][1,2]oxaborole-6-carboxylic acid (8) as white solid. Yield: 8.55 g (89%). ¹H NMR spectrum (300 MHz, DMSO-d6, δH): 13.25 (bs, 1H); 9.54 (s, 1H); 8.51 (s, 1H); 8.20 (s, 1H); 7.22 (t, J=55.1 Hz, 1H); 5.19 (s, 2H).

Solution of pentafluorophenol (8.28 g, 45.0 mmol), 4-(difluoromethyl)-1-hydroxy-1,3-dihydrobenzo[c][1,2]oxaborole-6-carboxylic acid (8, 8.55 g, 37.5 mmol) and N-(3-dimethylaminopropyl)-N-ethylcarbodiimide hydrochloride (EDC.HCl, 10.1 g, 52.5 mmol) in dichloromethane (100 mL) was stirred at ambient temperature for 3 hours. The reaction mixture was evaporated, dissolved in ethyl acetate (200 mL) and washed with 1 M aqueous solution of hydrochloric acid (3×200 mL) and brine (1×200 mL). Organic layer was separated, dried over anhydrous sodium sulfate, filtered and evaporated. The crude product 9 was recrystallized from hot cyclohexane (300 mL) and ethyl acetate (30 mL) to give the pentafluorophenyl 4-(difluoromethyl)-1-hydroxy-1,3-dihydrobenzo[c][1,2]oxaborole-6-carboxylate (9) as white solid. Yield: 8.20 g (56%). LC-MS: 395.5 (M+H)+.

Solution of the perfluorophenyl 4-(difluoromethyl)-1-hydroxy-1,3-dihydrobenzo[c][1,2]oxaborole-6-carboxylate (9, 8.20 g, 20.8 mmol), (2-aminoethyl)glycine (10, 1.23 g, 10.4 mmol) and triethylamine (14.5 mL, 104 mmol) in tetrahydrofuran (40 mL) and water (20 mL) was stirred at ambient temperature overnight. Tetrahydrofuran was then evaporated and 1 M aqueous solution of potassium hydrogen sulfate (30 mL) was added to the residue. This mixture was extracted with ethyl acetate (2×100 mL). Organic layers were combined, dried over anhydrous sodium sulfate, filtered and evaporated. The crude product 11 was dissolved in ethyl acetate (10 mL) and precipitated with cyclohexane (100 mL). The precipitate was filtered, washed with cyclohexane (50 mL) and freeze-dried to afford N-(4-(difluoromethyl)-1-hydroxy-1,3-dihydrobenzo[c][1,2]oxaborole-6-carbonyl)-N-(2-(4-(difluoromethyl)-1-hydroxy-1,3-dihydrobenzo[c][1,2]oxaborole-6-carboxamido)ethyl)glycine (11) as white solid. Yield: 4.59 g (82%). ¹H NMR spectrum (300 MHz, DMSO-d6, δH): 12.87 (bs, 1H); 9.66-9.33 (m, 2H); 8.95-6.68 (m, 7H); 5.15 (d, J=11.9 Hz, 4H); 4.39-3.94 (m, 2H); 3.76-3.37 (m, 4H). LC-MS: 539.1 (M+H)+.

### Example 39: 1-(tert-Butyl)-5-(2,5-dioxopyrrolidin-1-yl)-(2-((oxobis(3-(4,4,5,5-tetramethyl-1,3,2-dioxaborolan-2-yl)-5-(trifluoromethyl)phenyl)-λ⁶-sulfanylidene)amino)acetyl-L-glutamate

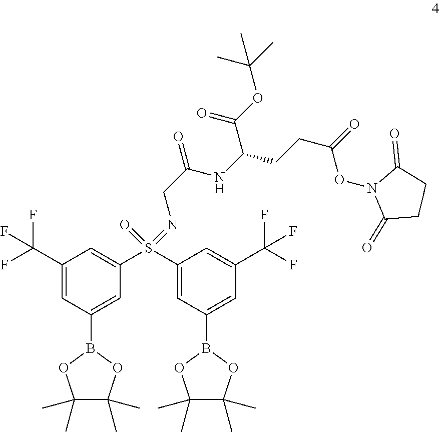 [SMILES: CC(C)(C)OC(=O)[C@H](CCC(=O)ON1C(=O)CCC1=O)NC(=O)CN=S(=O)(c1cc(B2OC(C)(C)C(C)(C)O2)cc(C(F)(F)F)c1)c1cc(B2OC(C)(C)C(C)(C)O2)cc(C(F)(F)F)c1]

Dry dichloromethane (37 mL) and triethylamine (1.53 mL, 11.0 mmol) were subsequently added to 2,5-dioxopyrrolidin-1-yl-2-((oxobis(3-(4,4,5,5-tetramethyl-1,3,2-dioxaborolan-2-yl)-5-(trifluoromethyl)phenyl)-λ⁶-sulfanylidene)amino)acetate (1, 2.78 g, 3.66 mmol) prepared in example 33 and (S)-4-amino-5-(tert-butoxy)-5-oxopentanoic acid (2, H-Glu-OtBu, 891 mg, 4.39 mmol). The mixture was sonicated to give a solution which was stirred at room temperature for 6 hours. The volatiles were removed in vacuo and the residue re-dissolved in ethyl acetate (200 mL). The resulting solution was washed with 0.5 M aqueous solution of hydrochloric acid (3×50 mL) and brine (50 mL), dried over anhydrous sodium sulfate and evaporated to dryness. The residue was re-dissolved in ethyl acetate (50 mL) and a solution of pinacol (432 mg, 3.66 mmol) in ethyl acetate (20 mL) was added. The resulting solution was evaporated in vacuo to give (S)-5-(tert-butoxy)-5-oxo-4-(2-((oxobis(3-(4,4,5,5-tetramethyl-1,3,2-dioxaborolan-2-yl)-5-(trifluoromethyl)phenyl)-λ⁶-sulfanylidene)amino)acetamido)pentanoic acid (3) as pale yellow foam. Yield: 3.07 g (99%).

¹H NMR spectrum (300 MHz, CDCl₃, δH): 8.57 (d, J=12.7 Hz, 2H); 8.40 (dd, J=8.3 and 0.7 Hz, 2H); 8.25 (s, 2H); 7.96 (d, J=8.1 Hz, 1H); 4.57 (m, 1H); 3.71 (dd, J=22.9 and 17.4 Hz, 2H); 2.53-2.43 (m, 2H); 2.37-2.24 (m, 1H); 2.15-2.02 (m, 1H); 1.47 (s, 9H); 1.37 (s, 24H). ¹⁹F NMR spectrum (282 MHz, CDCl₃, δF): −62.64 (s). LC-MS: 683.4 (M−2×pinacol−H)−.

N,N-Disuccinimidyl carbonate (DSC, 1.84 g, 7.19 mmol) and pyridine (0.58 mL, 7.19 mmol) were subsequently added to a solution of (S)-5-(tert-butoxy)-5-oxo-4-(2-((oxobis(3-(4,4,5,5-tetramethyl-1,3,2-dioxaborolan-2-yl)-5-(trifluoromethyl)phenyl)-λ⁶-sulfanylidene)amino)-acetamido)pentanoic acid (3, 3.05 g, 3.59 mmol) in dry acetonitrile (18 mL) and the mixture was sonicated to form a fine suspension. The resulting suspension was stirred at room temperature overnight to give a clear solution. The solution was evaporated to dryness and the residue was partitioned between ethyl acetate (250 mL) and 0.5 M aqueous solution of hydrochloric acid (100 mL). The phases were separated; the organic one was washed with 0.5 M aqueous solution of hydrochloric acid (4×100 mL) and brine (70 mL); dried over anhydrous sodium sulfate and evaporated to dryness. The residue was dissolved in dichloromethane (40 mL), followed by addition of pinacol (636 mg, 5.39 mmol). The solvent was removed in vacuo and the residue was evaporated from dichloromethane (50 mL). The resulting foam was triturated with cyclohexane (3×50 mL); the resulting semi-solid was decanted, dissolved in dichloromethane (50 mL) and evaporated to dryness in vacuo. The residue was evaporated from dichloromethane (3×50 mL) and dried in vacuo to afford the title compound (4) as white foam. Yield: 2.82 g (83%). ¹H NMR spectrum (300 MHz, CDCl₃, δH): 8.57 (s, 1H); 8.51 (s, 1H); 8.43 (s, 1H); 8.31 (s, 1H); 8.25 (s, 1H); 8.24 (s, 1H); 7.77 (d, J=7.9 Hz, 1H); 4.60 (m, 1H); 3.73 (dd, J=39.6 and 17.3 Hz, 2H); 2.82 (s, 4H); 2.79-2.62 (m, 2H); 2.43-2.30 (m, 1H); 2.20-2.06 (m, 1H); 1.49 (s, 9H); 1.36 (s, 24H).

¹⁹F NMR spectrum (282 MHz, CDCl₃, δF): −62.63 (s). LC-MS: 864.5 (M−pinacol+H)+, 946.7 (M+H)+.

### Example 40: (S)-5-(tert-Butoxy)-4-(2-(1-hydroxy-N-(2-(1-hydroxy-4-(trifluoromethyl)-1,3-dihydrobenzo[c][1,2]oxaborole-6-carboxamido)ethyl)-4-trifluoromethyl)-1,3-dihydrobenzo[c][1,2]oxaborole-6-carboxamido)acetamido)-5-oxopentanoic acid

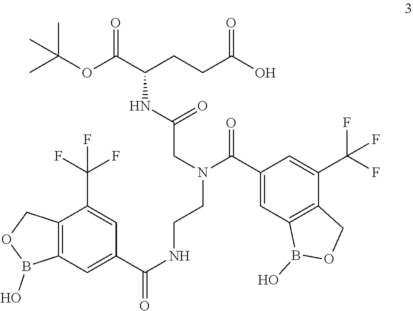 [SMILES: CC(C)(C)OC(=O)[C@H](CCC(=O)O)NC(=O)CN(CCNC(=O)c1cc2c(c(C(F)(F)F)c1)COB2O)C(=O)c1cc2c(c(C(F)(F)F)c1)COB2O]

2-Chlorotrityl chloride resin 100-200 mesh 1.5 mmol/g (1, 4.39 g, 6.59 mmol) was left to swell in dry dichloromethane (30 mL) for 30 minutes. A solution of (S)-2-(9H-fluoren-9-ylmethoxycarbonylamino) pentanedioic acid 1-tert-butyl ester (Fmoc-Glu-OtBu, 1.87 g, 4.39 mmol) and N,N-diisopropylethylamine (2.91 mL, 16.7 mmol) in dry dichloromethane (30 mL) was added to resin and the mixture was shaken overnight. Resin was filtered and treated with a solution of N,N-diisopropylethylamine (1.53 mL, 8.78 mmol) in methanol/dichloromethane mixture (4:1, 2×5 min, 2×40 mL). Then resin was washed with N,N-dimethylformamide (2×30 mL), dichloromethane (2×40 mL) and N,N-dimethylformamide (3×40 mL). Fmoc group was removed by treatment with 20% piperidine in N,N-dimethylformamide (1×5 min, 1×20 min, 2×40 mL). Resin was washed with N,N-dimethylformamide (3×40 mL), 2-propanol (2×40 mL) and dichloromethane (3×40 mL). Solution of N-(((9H-fluoren-9-yl)methoxy)carbonyl)-N-(2-((((9H-fluoren-9-yl)methoxy)carbonyl)amino)ethyl)glycine (Fmoc-AEG(Fmoc)-OH, 3.71 g, 6.59 mmol), 1-((dimethylamino)(dimethyliminio)methyl)-1H-[1,2,3]triazolo[4,5-b]pyridine 3-oxide hexafluorophosphate (HATU, 2.50 g, 6.59 mmol) and 2,4,6-trimethylpyridine (1.57 mL, 11.9 mmol) in N,N-dimethylformamide (40 mL) was added to resin and mixture was shaken for 2 hours. Resin was filtered and washed with N,N-dimethylformamide (2×40 mL), dichloromethane (2×40 mL) and N,N-dimethylformamide (2×40 mL). Fmoc group was removed by treatment with 20% piperidine in N,N-dimethylformamide (1×5 min, 1×30 min, 2×40 mL). Resin was washed with N,N-dimethylformamide (3×40 mL), 2-propanol (2×40 mL) and dichloromethane (3×40 mL). Solution of pentafluorophenyl 1-hydroxy-4-(trifluoromethyl)-1,3-dihydrobenzo[c][1,2]oxaborole-6-carboxylate (2, 5.43 g, 13.2 mmol) and triethylamine (4.90 mL, 35.1 mmol) in N,N-dimethylformamide (40 mL) was added to resin and mixture was shaken overnight. Resin was filtered and washed with N,N-dimethylformamide (6×40 mL) and dichloromethane (10×50 mL). The product was cleaved from resin by treatment with 2,2,2-trifluoroethanol (60 mL) for 16 hours. Resin was filtered off and washed with dichloromethane (4×50 mL). Solvents were evaporated; the residue was extracted with 1 M aqueous solution of potassium hydrogen sulfate (50 mL) and ethyl acetate (2×70 mL), organic phases were dried over anhydrous sodium sulfate, filtered and the solvent was evaporated. Crude product was precipitated from ethyl acetate/cyclohexane (1:10, 40 mL), purified by column chromatography (Silicagel 60, 0.063-0.200 mm; eluent: acetonitrile/water 10:1) and freeze-dried to give (S)-5-(tert-butoxy)-4-(2-(1-hydroxy-N-(2-(1-hydroxy-4-(trifluoromethyl)-1,3-dihydrobenzo[c][1,2]oxaborole-6-carboxamido)ethyl)-4-(trifluoromethyl)-1,3-dihydrobenzo[c][1,2]oxaborole-6-carboxamido)acetamido)-5-oxopentanoic acid (3) as white solid. Yield: 1.50 g (45%). ¹H NMR spectrum (300 MHz, AcOD-d4, δH): 8.44 (s, 1H); 8.24 (s, 1H); 8.05 (s, 1H); 7.77 (s, 1H); 5.25 (d, J=17.1 Hz, 4H); 4.70-4.25 (m, 3H); 4.03-3.67 (m, 4H); 2.49 (bs, 2H); 2.22 (bs, 1H); 1.49 (s, 9H). LC-MS: 760.3 (M+H)+.

### Example 41: 1-hydroxy-1,3-dihydrobenzo[c][1,2]oxaborole-6-carboxylic acid

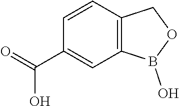 [SMILES: O=C(O)c1ccc2c(c1)B(O)OC2]

N-Bromosuccinimide (NBS, 88.1 g, 495 mmol) was added to a cold suspension (10° C.) of 4-methybenzonitrile (58.6 g, 500 mmol) in 50% aqueous sulfuric acid (270 mL). The reaction mixture was stirred for 40 hours at 10 C in the dark. After that suspension was filtered and filter cake was washed with water (100 ml) and dissolved in ethyl acetate (800 mL). Solution of crude product in ethyl acetate was washed with water (400 mL), saturated aqueous solution of sodium hydrogen carbonate (2×400 mL) and brine (200 mL). Organic solution was dried over anhydrous magnesium sulfate and evaporated to dryness to give crude 3-bromo-4-methylbenzonitrile as yellow crystals. The product was used in the next step without purification. Yield: 90.70 g (92%). RF (SiO2, hexanes/ethyl acetate 9:1): 0.45. ¹H NMR spectrum (300 MHz, CDCl3, δH): 7.82 (d, J=1.5 Hz, 1H); 7.50 (dd, J=7.9 and 1.7 Hz, 1H); 7.34 (d, J=7.9, 1H); 2.47 (s, 3H).

Benzoyl peroxide (1 g) and N-bromosuccinimide (NBS, 96.3 g, 541 mmol) were added to a solution of 3-bromo-4-methylbenzonitrile (90.7 g, 463 mmol) in tetrachloromethane (1.00 L). The mixture was refluxed overnight. After that the reaction mixture was cooled down, diluted with dichloromethane (500 mL) and extracted with water (2×500 mL). Organic solution was dried over anhydrous magnesium sulfate and evaporated to dryness to give crude 3-bromo-4-(bromomethyl)benzonitrile as brown oil. Yield: 135 g. RF (SiO2, hexanes/ethyl acetate 9:1): 0.45.

Potassium acetate (98.1 g, 1.00 mol) was added to a cool (4° C.) solution of the crude above 3-bromo-4-(bromomethyl)benzonitrile (135 g) in acetonitrile (700 mL). The mixture was stirred at 70° C. for 24 hours. The mixture was evaporated and the residue was diluted ethyl acetate (800 mL) and extracted with water (2×500 mL). The organic phase was dried over magnesium sulfate and evaporated to dryness. The residue was purified by flash column chromatography (Silicagel 60, 0.040-0.060 mm; eluent:hexanes/ethyl acetate 20:1 to 5:1) to give 2-bromo-4-cyanobenzyl acetate as white crystals. Yield: 60.90 g (52% over two steps). RF (SiO2, hexanes/ethyl acetate 4:1): 0.30. ¹H NMR spectrum (300 MHz, CDCl3, δH): 7.87 (d, J=1.5 Hz, 1H); 7.64 (dd, J=8.1 and 1.7 Hz, 1H); 7.53 (d, J=8.1 Hz, 1H); 5.22 (s, 2H); 2.19 (s, 3H).

Under argon atmosphere, 2-bromo-4-cyanobenzyl acetate (60.0 g, 236 mmol), potassium acetate (46.3 g, 472 mmol), bis(pinacotato)diboron (65.9 g, 259 mmol) and [1,1-bis(diphenylphosphino)ferrocene]dichloropalladium(II) complex with dichloromethane (5 g) were dissolved in degassed 1,4-dioxane (800 mL) and the mixture was refluxed for 18 hours, After that the mixture was filtered and filtrate was evaporated and the residue re-dissolved in ethyl acetate (800 mL). The solution was washed with water (2×400 mL) and brine (400 mL). The organic phase was dries over magnesium sulfate and evaporated to dryness. The residue was purified by column chromatography (Silicagel 60, 0.040-0.060 mm; eluent: hexanes/ethyl acetate 8:1) to give 4-cyano-2-(4,4,5,5-tetramethyl-1,3,2-dioxaborolan-2-yl)benzyl acetate white crystals. Yield: 48.80 g (69%). RF (SiO2, hexanes/ethyl acetate 4:1): 0.35. ¹H NMR spectrum (300 MHz, CDCl3, δH): 8.13 (d, J=1.7 Hz, 1H); 7.71 (dd, J=7.9 and 1.9 Hz, 1H); 7.49 (d, J=8.1 Hz, 1H); 5.42 (s, 2H); 2.13 (s, 3H).

A solution of sodium hydroxide (13.1 g, 327 mmol) in methanol (300 mL) was added dropwise to a solution of 4-cyano-2-(4,4,5,5-tetramethyl-1,3,2-dioxaborolan-2-ly)benzyl acetate (44.8 g, 149 mmol) in methanol (300 mL) at 30 C. The reaction mixture was stirred for additional 2 hours. The solvent was evaporated and the residue was dissolved in tetrahydrofuran (200 mL). 2 M Aqueous hydrochloric acid (660 mL) was added and the resulting suspension was stirred for 10 minutes. The suspension was cooled down to 10° C. and filtered. The filter cake was washed by water (100 mL) and n-hexane (100 mL) to give 1-hydroxy-1,3-dihydrobenzo[c][1,2]oxaborole-6-carbonitrile as white powder. Yield: 20.15 g (85%). ¹H NMR spectrum (300 MHz, DMSO-d6, δH): 9.55 (bs, 1H); 8.09 (s, 1H); 7.90 (d, J=8.1 Hz, 1H); 7.63 (d, J=7.9 Hz, 1H); 5.07 (s, 2H).

A suspension of 1-hydroxy-1,3-dihydrobenzo[c][1,2]oxaborole-6-carbonitrile (20.15 g, 127 mmol) in conc. hydrochloric acid (1.50 L) was refluxed for 24 hours and cooled down to 10° C. The suspension was filtered and filter cake washed with water (300 mL). Filter cake was suspended in water (500 mL) and freeze-dried. The residue was suspended in dichloromethane (500 mL) and filtered. Filter cake was wash with dichloromethane (200 mL) and dried in vacuo to give 1-hydroxy-1,3-dihydrobenzo[c][1,2]oxaborole-6-carboxylic acid as white powder. Yield: 12.30 g (55%). ¹H NMR spectrum (300 MHz, DMSO-d6, δH): 12.92 (s, 1H); 9.36 (s, 1H); 8.37 (s, 1H); 8.04 (dd, J=7.9 and 0.9 Hz, 1H); 7.52 (d, J=8.1 Hz, 1H); 5.05 (s, 2H). LC-MS m/z: 178.2 (M+H).

### Example 42: 1-Hydroxy-4-(trifluoromethyl)-1,3-dihydrobenzo[c][1,2]oxaborole-6-carboxylic acid

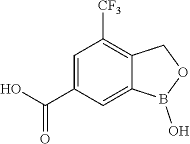 [SMILES: O=C(O)c1cc2c(c(C(F)(F)F)c1)COB2O]

1-Hydroxy-4-(trifluoromethyl)-1,3-dihydrobenzo[c][1,2]oxaborole-6-carboxylic acid was prepared as described in Example 28.

### Example 43: 4-fluoro-1-hydroxy-1,3-dihydrobenzo[c][1,2]oxaborole-6-carboxylic acid

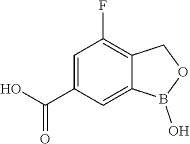 [SMILES: O=C(O)c1cc(F)c2c(c1)B(O)OC2]

Intensively stirred solution of 3-fluoro-4-methylbenzoic acid (1, 61.7 g, 400 mmol) in sulfuric acid (96%, 400 mL) was cooled by external ice water bath and N-bromosuccinimide (72.0 g, 405 mmol) was added in three portions during 20 minutes. The mixture was stirred at room temperature for 4 hours then another portion of N-bromosuccinimide (72.0 g, 405 mmol) was added at once and the whole mixture was stirred at room temperature overnight. Resulting suspension was diluted with ice water (3.00 L) and stirred for 10 minutes. The solid was filtered off, washed with water (200 mL), triturated with water (3×600 mL) and sucked off as much as possible. Wet solid was suspended in water (400 mL), stirred at room temperature and solution of sodium hydroxide (50.0 g, 1.25 mol in 200 mL water) was added. Resulting solution was heated to 40° C. overnight. Filtration of slightly cloudy solution afforded clear yellowish filtrate to which solution of potassium bisulfate (180 g, 1.32 mol in 400 mL water) was added. White precipitate was extracted with a mixture of dichloromethane/tetrahydrofuran 4:1 (2×500 mL). Organic extracts were dried over anhydrous sodium sulfate and evaporated to dryness to give white solid residue. Thionyl chloride (30.0 mL, 413 mmol) was added to stirred cooled (−78° C.) suspension of this residue in anhydrous methanol (500 mL). Reaction mixture was allowed to warm to room temperature and then heated to 60 C overnight. The solution was cooled to room temperature and kept 4° C. overnight. Crystalline material was filtered off washed by methanol (2×50 mL), tert-butyl methyl ether (2×50 mL) and dried in vacuo to afford methyl 2,3-dibromo-5-fluoro-4-methylbenzoate (2) as colorless crystals. Yield: 78.2 g (60%). ¹H NMR spectrum (300 MHz, CDCl3, δH): 7.37 (d, J=9.0 Hz, 1H); 3.94 (s, 3H); 2.46 (d, J=2.3 Hz, 3H). LC-MS m/z: 327.2 (M+H)+.

A suspension of fine powdered copper (44.0 g, 692 mmol) and methyl 2,3-dibromo-5-fluoro-4-methylbenzoate (2, 75.2 g, 231 mmol) in propionic acid (100 mL) was stirred and heated at 85-90° C. for 6 hours, cooled to room temperature and diluted with mixture of cyclohexane/toluene (3:1, 800 mL). Reaction mixture was washed with water (3×200 mL), 10% aqueous solution of potassium bisulfate (2×200 mL) and brine (2×300 mL). Organic solution was dried over anhydrous sodium sulfate and evaporated to dryness to give yellowish oil which was purified by flash column chromatography (Silicagel 60, 0.040-0.060 mm; eluent:cyclohexane/toluene 3:1) to afford methyl 3-bromo-5-fluoro-4-methylbenzoate (3) as colorless crystals. Yield: 52.5 g (92%). ¹H NMR spectrum (300 MHz, CDCl3, δH): 7.51 (s, 1H); 7.37 (d, J=9.0 Hz, 1H); 3.86 (s, 3H); 2.37 (d, J=2.4 Hz, 3H). LC-MS m/z: 347.3 (M+H)+.

Methyl 3-bromo-5-fluoro-4-methylbenzoate (3, 51.9 g, 210 mmol) was dissolved in anhydrous 1,4-dioxane (400 mL), anhydrous potassium acetate (65.3 g, 666 mmol) and bis(pinacolato)diboron (4, 75.1 g, 296 mmol) was added at room temperature and this mixture was degassed. 1,1-Bis(diphenylphosphino)ferrocene]dichloropalladium(II) (1.88 g, 2.57 mmol) was added and the mixture was heated to 75° C. in an argon atmosphere for 40 hours. The mixture was concentrated under reduced pressure and dissolved in toluene (1.1 L) and extracted with water (2×200 mL). Organic solution was dried using anhydrous sodium sulfate, evaporated under reduced pressure and then purified by flash column chromatography (Silicagel 60, 0.040-0.063 mm; eluent:toluene/ethyl acetate 9:1) to afford methyl 3-fluoro-4-methyl-5-(4,4,5,5-tetramethyl-1,3,2-dioxaborolan-2-yl)benzoate (5) as white solid. Yield: 50.0 g (81%). ¹H NMR spectrum (300 MHz, CDCl₃, dH): 8.20 (s, 1H); 7.70 (d, J=10.0 Hz, 1H); 3.85 (s, 3H); 2.50 (s, 3H); 1.36 (s, 12H). LC-MS m/z: 295.4 (M+H)+.

Azobisisobutyronitrile (AIBN, 0.86 g, 5.20 mmol) and N-bromosuccinimide (NBS, 25.4 g, 143 mmol) were added to a solution of methyl 3-fluoro-4-methyl-5-(4,4,5,5-tetramethyl-1,3,2-dioxaborolan-2-yl)benzoate (5, 40.0 g, 136 mmol) in 1,2-dichloroethane (200 mL). The mixture was refluxed overnight. Reaction mixture was cooled to room temperature, diluted with dichloromethane (500 mL) and extracted with water (2×500 mL). Organic solution was dried over anhydrous magnesium sulfate and evaporated to dryness to give methyl 4-(bromomethyl)-3-fluoro-5-(4,4,5,5-tetramethyl-1,3,2-dioxaborolan-2-yl)benzoate (6) as yellowish crystals. The product was used in the next step without further purification. Yield: 35.5 g (70%). LC-MS m/z: 373.4 (M+H)+.

Methyl 4-(bromomethyl)-3-fluoro-5-(4,4,5,5-tetramethyl-1,3,2-dioxaborolan-2-yl)benzoate (6, 7.46 g, 20.0 mmol) stirred with 2.5 M aqueous solution of sodium hydroxide (40.0 mL, 100 mmol) at room temperature overnight. 6 M aqueous solution of hydrochloric acid (20.0 mL, 120 mmol) was added and the mixture was stirred for 30 minutes and kept 4° C. overnight. White precipitate was collected by filtration and washed with water (2×100 mL) and air dried to afford 4-fluoro-1-hydroxy-1,3-dihydrobenzo[c][1,2]oxaborole-6-carboxylic acid (7) as a white solid which was used in the next step without further purification. Yield: 3.76 g (96%).

¹H NMR spectrum (300 MHz, DMSO-d6, δH): 12.8 (s, 1H); 9.57 (s, 1H); 8.20 (s, 1H); 7.72 (d, J=7.1 Hz, 1H); 5.14 (s, 2H). LC-MS m/z: 197.4 (M+H)+.

Preparation of Insulin Derivatives

LCMS analysis were performed using C18 column and 0.1% TFA in water as buffer A and 0.1% TFA in acetonitrile as buffer B.

LCMS of boron-insulin derivatives generally show dehydrated species as the main peaks:

[M+nH−2×m M_{water}]ⁿ⁺ for ionization state “n” and “m” number of boronic acids

[M+nH−1×m M_{water}]ⁿ⁺ for ionization state “n” and “m” number of boroxoles e.g. [M+5H−2×(4×18.015)]⁵⁺ for the penta-ionic state of a derivative with 4 boronic acids

Measured and calculated values for [M+4H−x(water)]⁴⁺ and [M+5H−x(water)]⁵⁺ are shown in table 2 (shown under Example B).

The insulin conjugates in the examples are drawn using the standard single letter abbreviations for the amino acids. The sulfur atoms of the cysteine residues are drawn out specifically to illustrate disulfide bridges. Residues that are modified by conjugation are drawn out to show exactly where in the relevant amino acid the modification has taken place. The N-terminals of insulin are denoted with small font H—, and the C-terminals are denoted with small font —OH, as is standard in peptide chemistry. H— and —OH are not used when a terminal residue is modified by conjugation, in which case the residues are drawn expanded, as explained above. Substitutions in human insulin are in some cases illustrated with a small font star (*).

Building blocks that were not already succinimidyl ester, were activated using HONSU/DIC or TSTU in acetonitrile or THF before conjugation with insulin.

### Example 101

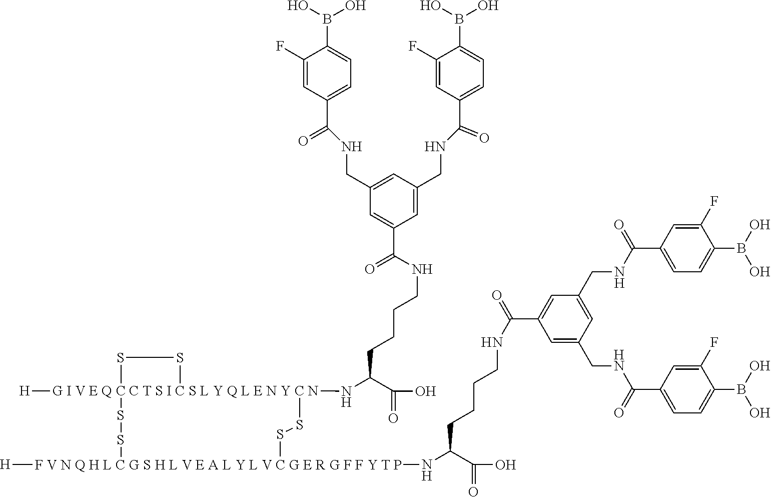

A22K desB30 human insulin (500 mg, 0.086 mmol) was dissolved in 0.1 M sodium carbonate (5 mL), pH 10.5. Building block of example 2 (146 mg, 0.189 mmol) was dissolved in MeCN (5 mL) and added to the mentioned insulin solution. pH was monitored and stayed near 10.5. After 30 mins, LCMS shows formation of the desired product. The mixture was diluted with 20% MeCN in water (11 mL) and pH was adjusted to 1.5 using TFA. The product was purified by reverse-phase HPLC (RP-HPLC) on C18 column using 0.1% TFA in water as buffer A and 0.1% TFA in acetonitrile as buffer B. The product was isolated by lyophilisation.

LCMS measured 1670.4 [M+4H−8× water]⁴⁺, calculated 1670.6, see table 2 (shown under Example B).

### Example 102

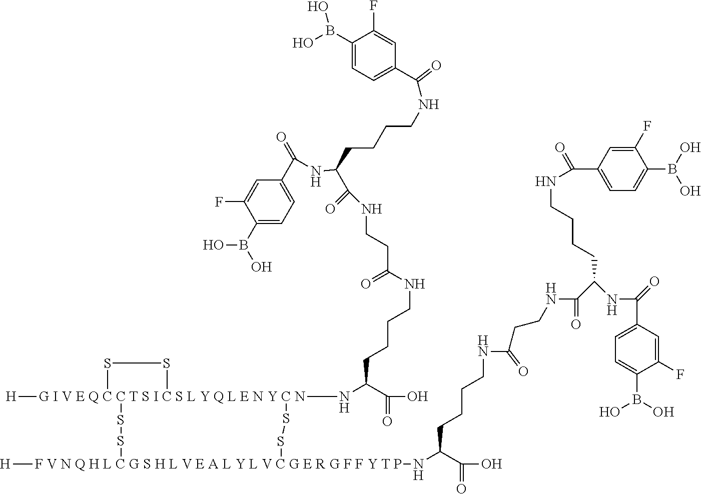

Insulin derivative of example 102 was prepared similarly to insulin derivative of example 101 from A22K desB30 human insulin and building block of example 3. LCMS of the product measured 1689.0 [M+4H−8× water]⁴⁺, calculated 1689.2.

### Example 103

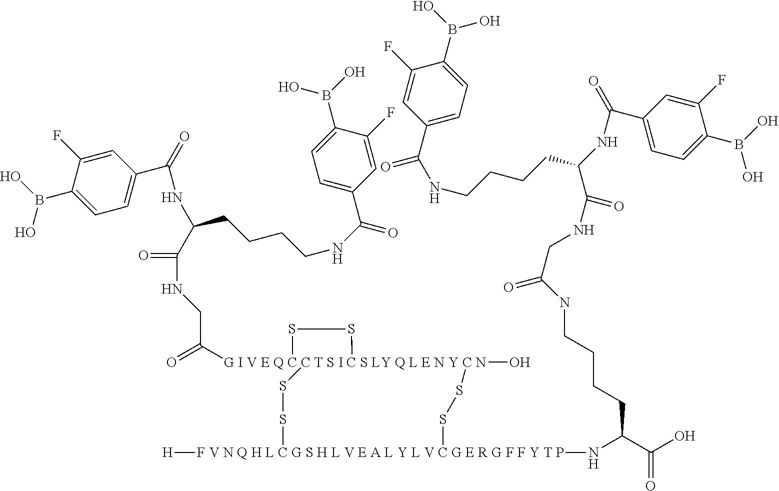

Insulin derivative of example 103 was prepared by dissolved desB30 human insulin (232 mg, 0.041 mmol) in DMSO and adding building block of example 4 (35.6 mg, 0.045 mmol) in DMSO along with NMM (1.22 mmol, 135.5 uL). The product was purified by reverse-phase HPLC (RP-HPLC) on C18 column using 0.1% TFA in water as buffer A and 0.1% TFA in acetonitrile as buffer B and isolated by lyophilisation. LCMS of the product measured 1663.0 [M+4H−4× water]⁴⁺, calculated 1664.1.

### Example 104

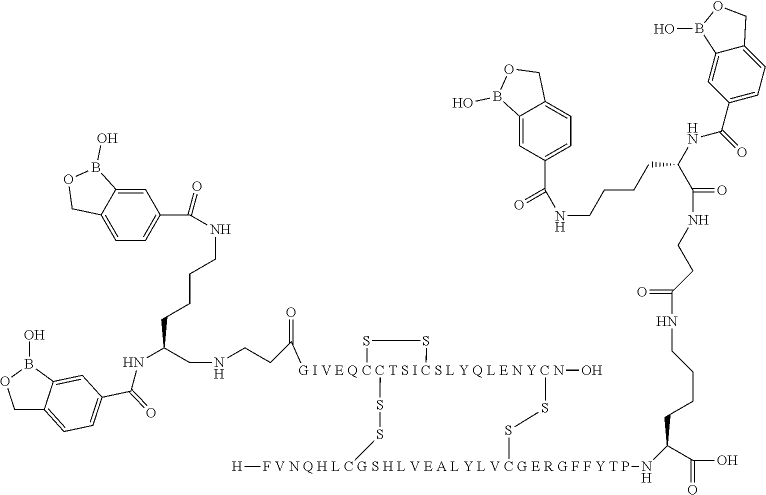

Insulin derivative of example 104 was prepared similarly to insulin derivative of example 103 from desB30 human insulin and an analogue of building block of example 4 made using 4-carboxy-benzoboroxole, lysine and beta-alanine.

### Example 105

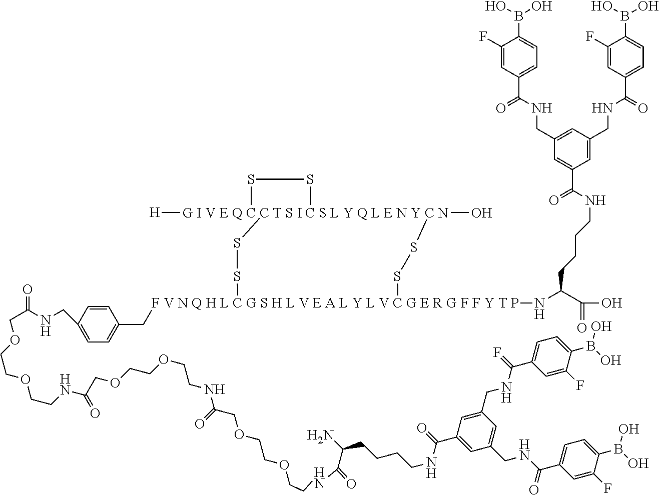

DesB30 human insulin (400 mg) was dissolved in 0.1M AcOH (5 mL) and pH was adjusted to 3.5 using 0.1N NaOH. A solution of aldehyde linker of Example 6 (200 mg) was dissolved in DMF (0.5 ml) and added. After stirring for 30 min, picoline borane (44 mg) dissolved in NMP (0.5 mL) was added. The reaction mixture was stirred overnight at RT. Water (20 mL) was added and pH was adjusted to 1 using 01. M HCl, and the product was purified by HPLC. The Boc groups on Lys in the extension were removed using TFA. The bis-Lys insulin intermediate (33 mg) was dissolved in 0.2 M Na₂CO₃ (0.400 mL) and pH was adjusted to 10.5. The diboronate succinimidyl ester of Example 2 (2.5 eq, 0.6 mg) was dissolved in acetonitrile (340 uL) and added to the mixture. The reaction was stirred for 10 min, the progress of the reaction monitored by LCMS, and the product isolated by HPLC similarly to Example 101. LCMS measured 1827.3 [M+4H−4× water]⁴⁺, calculated 1827.3.

### Example 106

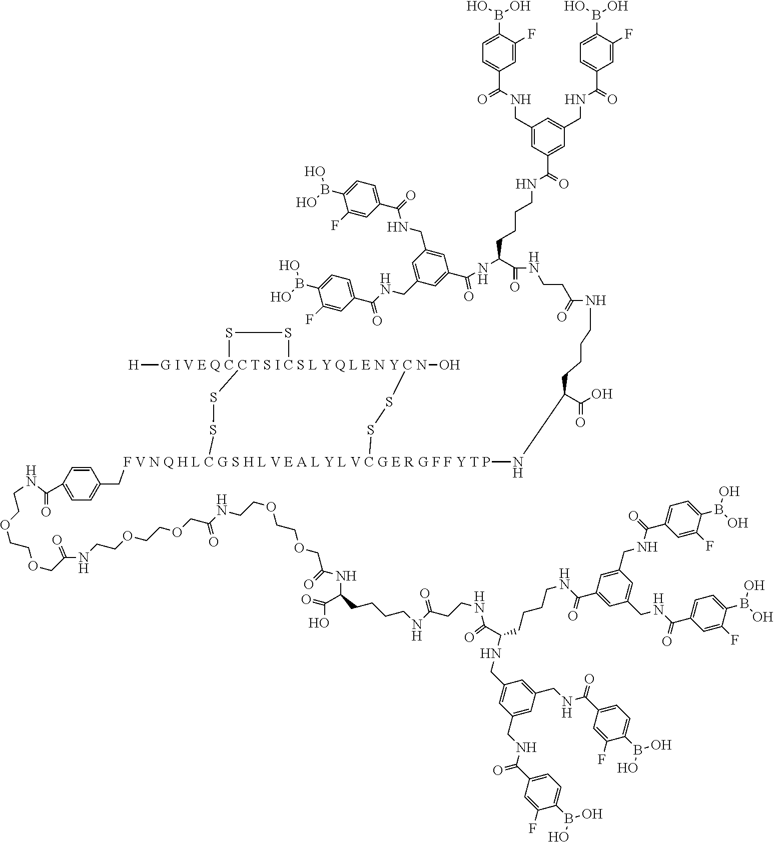

Example 106 was made similarly to example 105 using building block of example 7. LCMS measured 1724.3, calculated 1724.2.

### Example 107

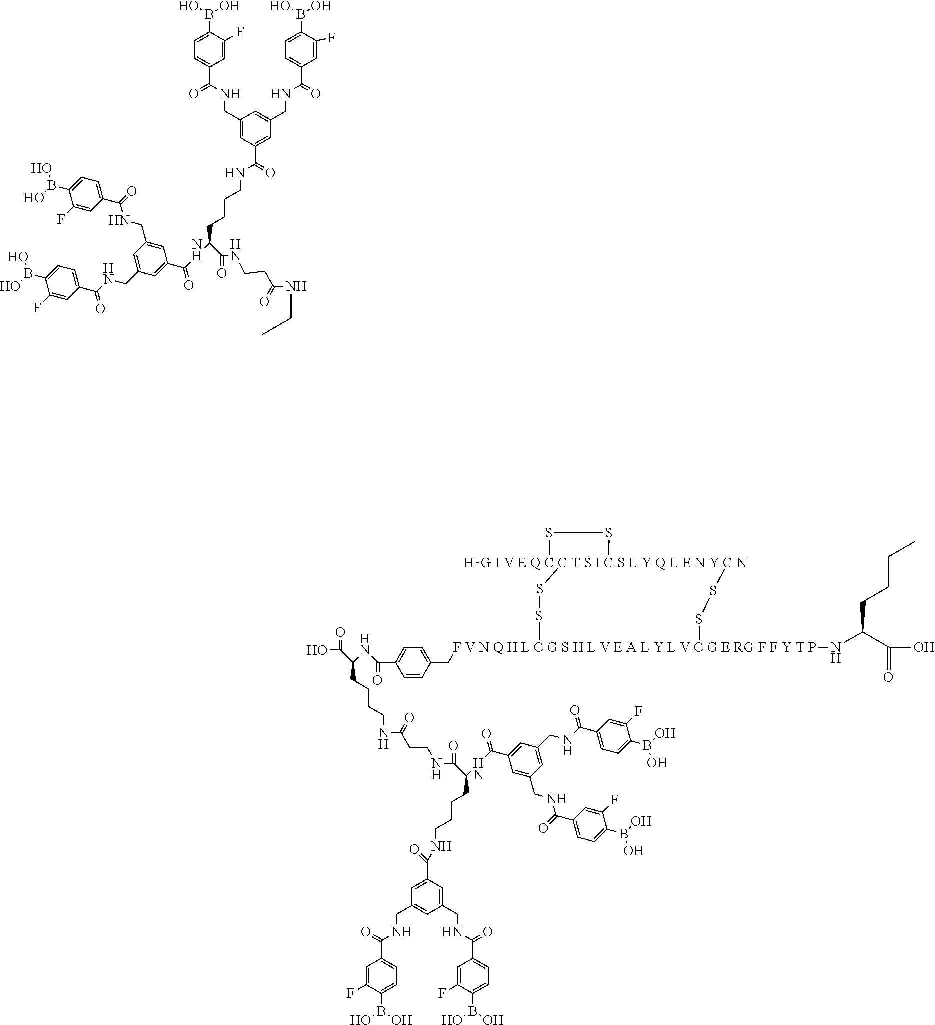

Example 107 was made similarly to example 105 using building block of example 7. LCMS measured 1640.8, calculated 1640.9.

### Example 108

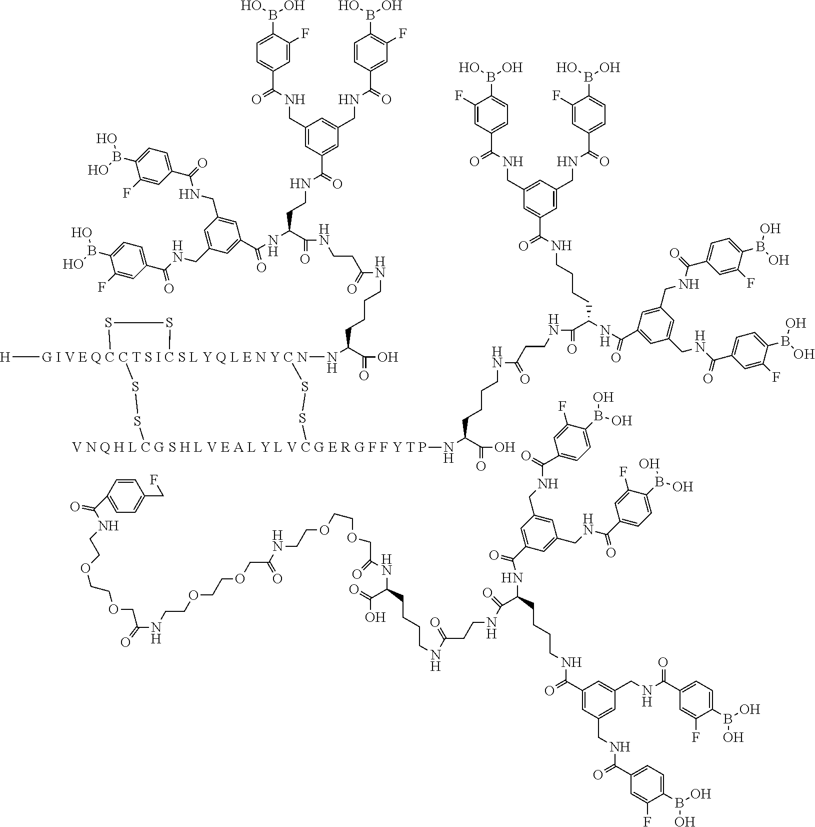

Example 109 was made similarly to example 107 from A22K desB30 human insulin and building block of example 7. LCMS measured 1987.5, calculated 1987.5.

### Example 109

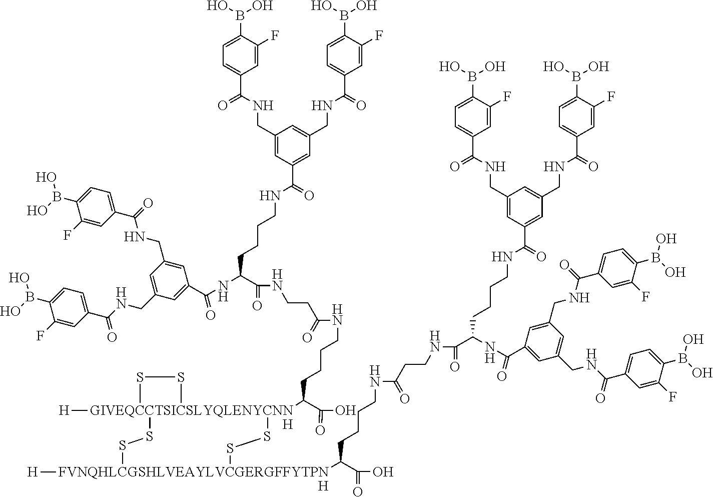

A22K desB30 human insulin (500 mg, 0.086 mmol) was dissolved in 0.2 M sodium carbonate buffer (6 mL), pH 10.8. Building block of example 7 (307 mg, 0.189 mmol) was dissolved in MeCN (6 mL). LCMS after 10 mins showed the expected product, which was purified by HPLC.

### Example 110

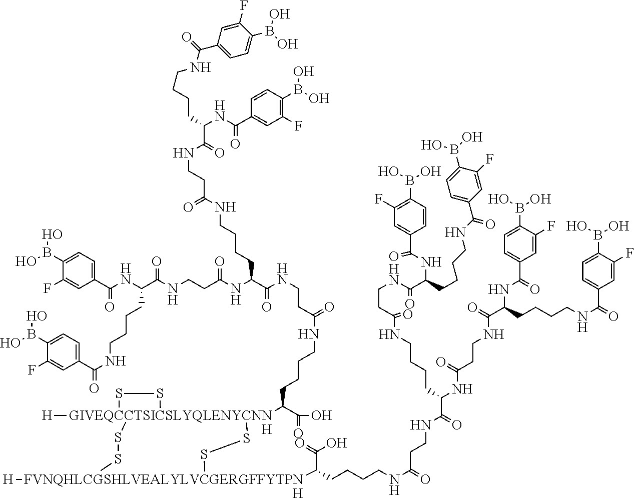

A22K desB30 human insulin (435 mg, 0.075 mmol) was dissolved in 0.2 M sodium carbonate buffer (10 mL), pH 10.8. Building block of example 8 (279 mg, 0.164 mmol) activated as succinimidyl ester (using TSTU/DIEA in MeCN) was dissolved in MeCN (6 mL). LCMS after 10 mins showed the expected product, which was purified by HPLC. LCMS measured 1643.7 [M+5H−8× water]⁵⁺, calculated 1643.7.

### Example 111

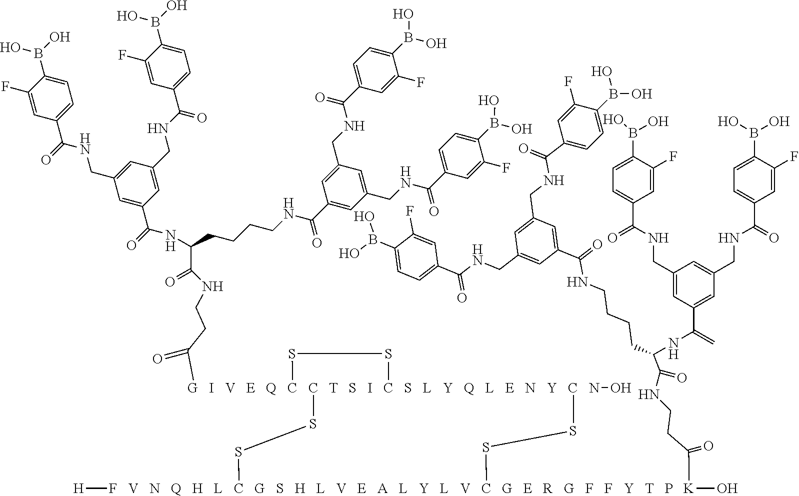

Example 111 was made similarly to example 103 from desB30 human insulin and building block of example 7.

### Example 112

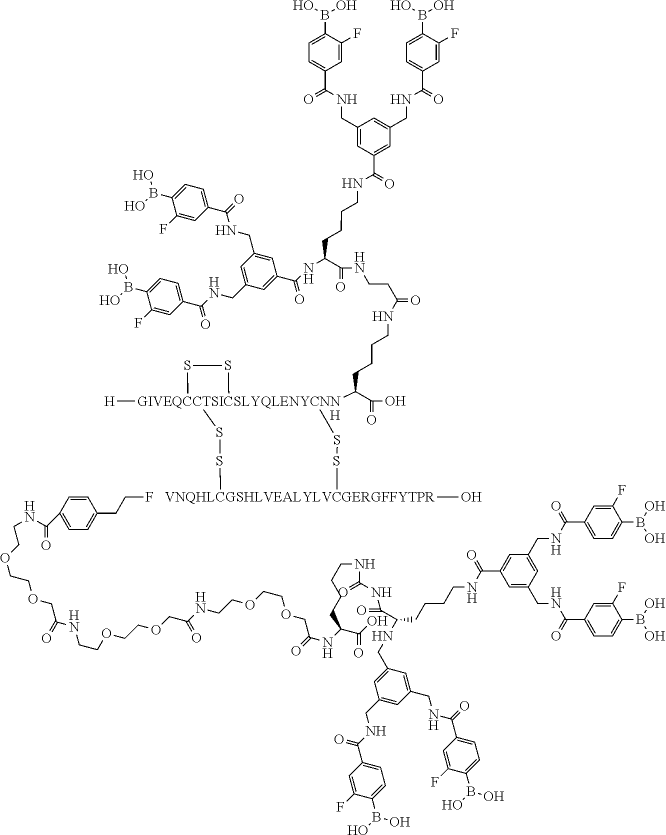

Example 112 was made similarly to example 105 from A22K B29R desB30 human insulin and building block of example 7.

### Example 113

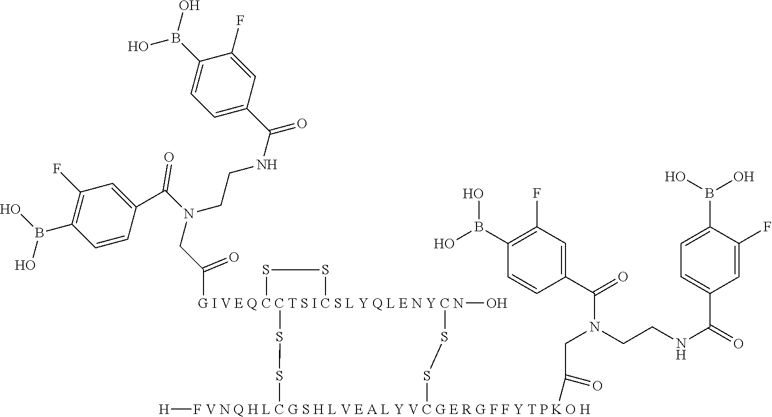

Example 113 was made similarly to example 101 from desB30 human insulin and building block of example 9.

### Example 114

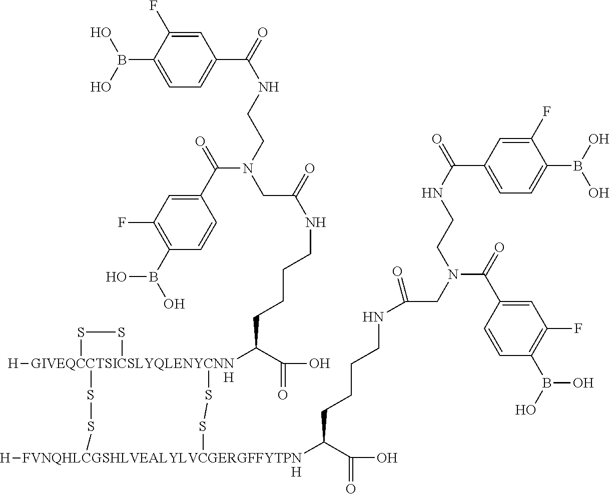

Example 114 was made similarly to example 101 from A22K desB30 human insulin and building block of example 9.

### Example 115

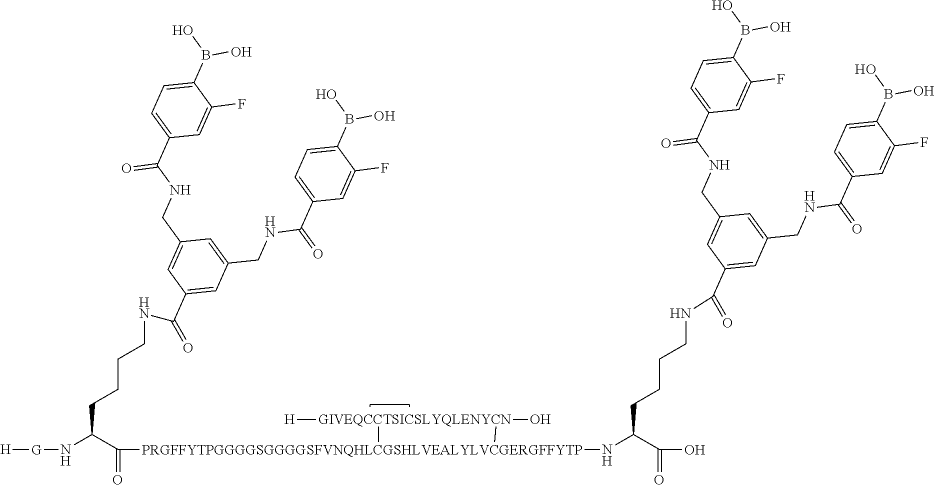

Example 115 was made similarly to example 101 from B1-GKPRGFFYTPGGGGSGGGGS desB30 human insulin and building block of example 3.

### Example 116

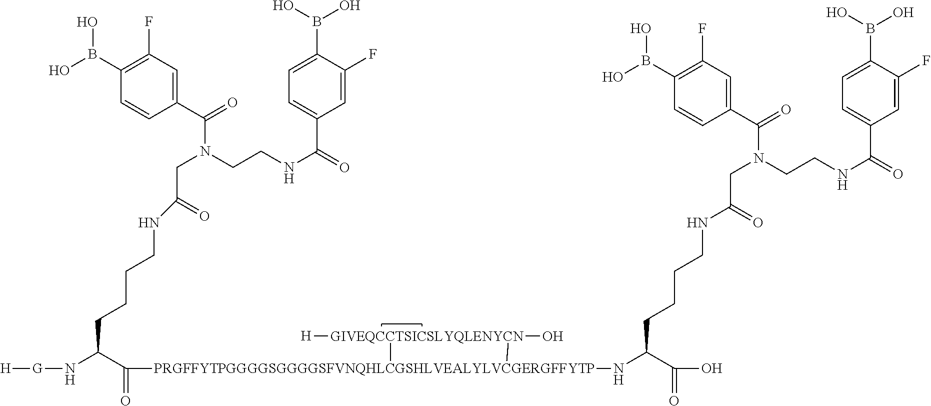

Example 116 was made similarly to example 101 from B1-GKPRGFFYTPGGGGSGGGGS desB30 human insulin and building block of example 9.

### Example 117

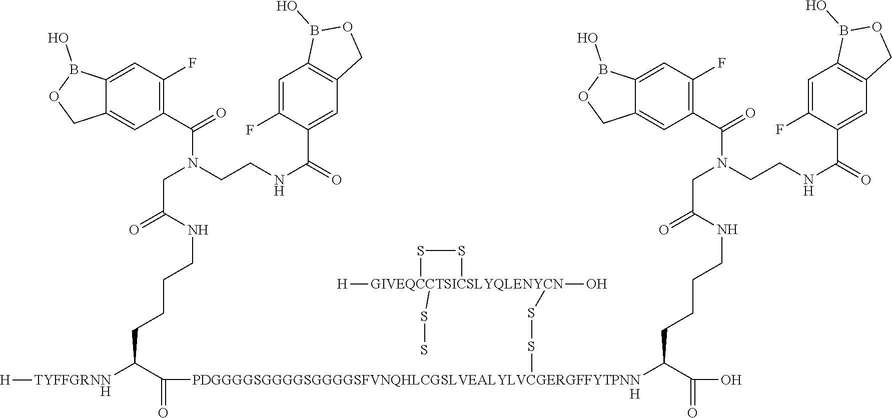

Example 117 was made similarly to example 101 from B1-TYFFGRKPDGGGGSGGGGSGGGGS desB30 human insulin and building block of example 10.

### Example 118

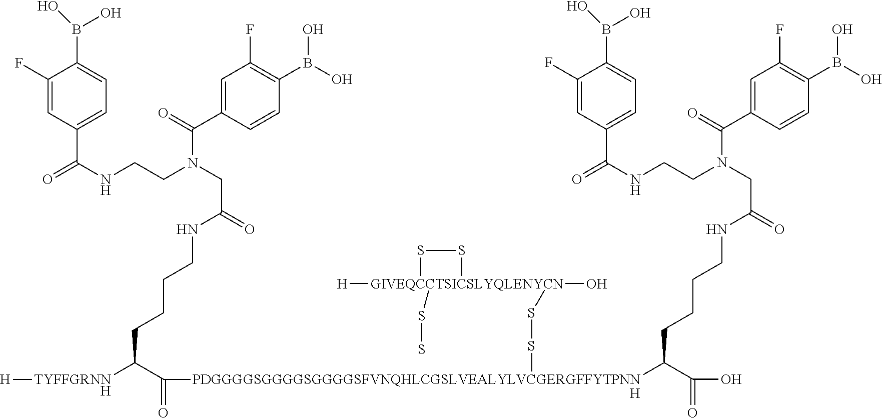

Example 118 was made similarly to example 101 from B1-TYFFGRKPDGGGGSGGGGSGGGGS desB30 human insulin and building block of example 9.

### Example 119

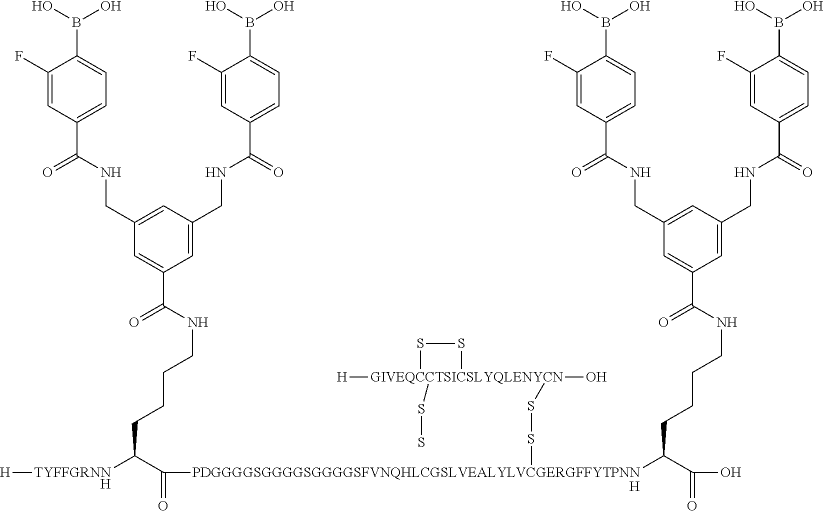

Example 119 was made similarly to example 101 from B1-TYFFGRKPDGGGGSGGGGSGGGGS desB30 human insulin and building block of example 2.

### Example 120

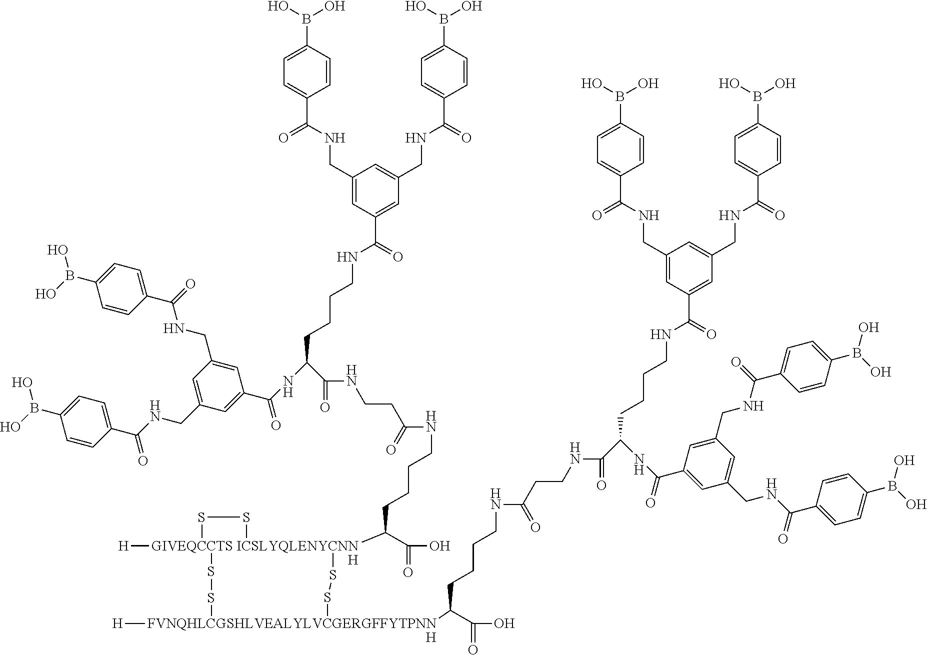

Example 120 was made similarly to example 101 from A22K desB30 human insulin and building block of example 11.

### Example 121

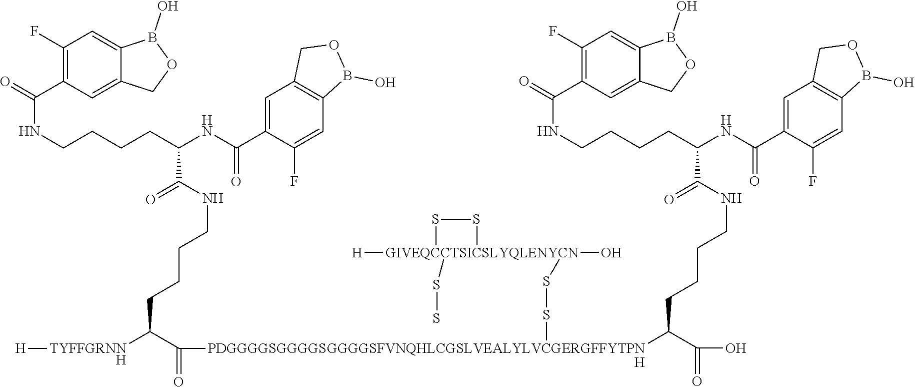

Example 121 was made similarly to example 101 from B1-TYFFGRKPDGGGGSGGGGSGGGGS desB30 human insulin and building block of example 10.

### Example 122

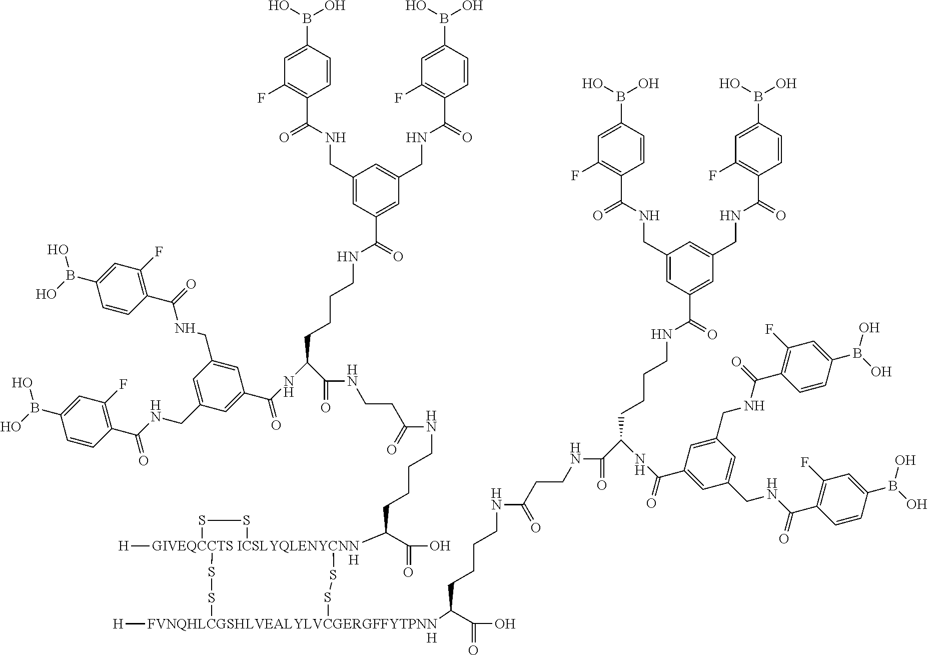

Example 122 was made similarly to example 101 from A22K desB30 human insulin and building block of example 12.

### Example 123

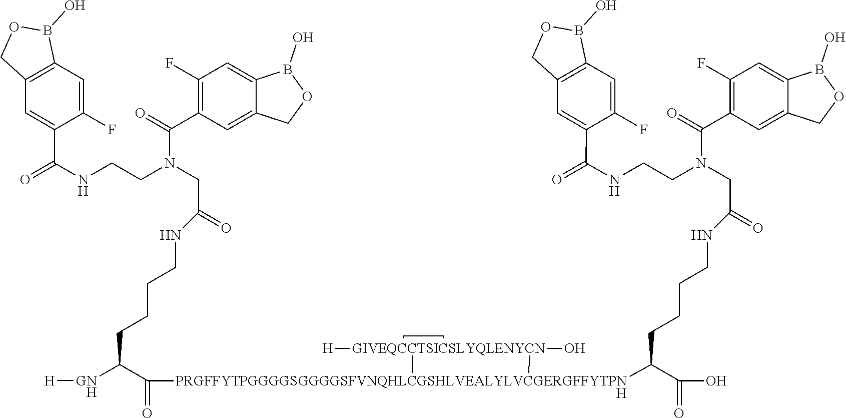

Example 123 was made similarly to example 101 from B1-GKPRGFFYTPGGGGSGGGGS desB30 human insulin and building block of example 10.

### Example 124

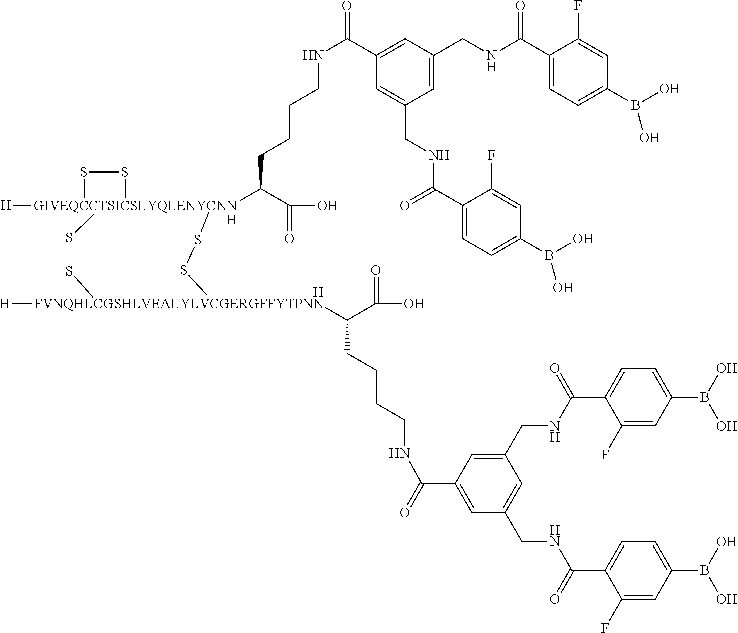

Example 124 was made similarly to example 101 from A22K desB30 human insulin and building block of example 13.

### Example 125

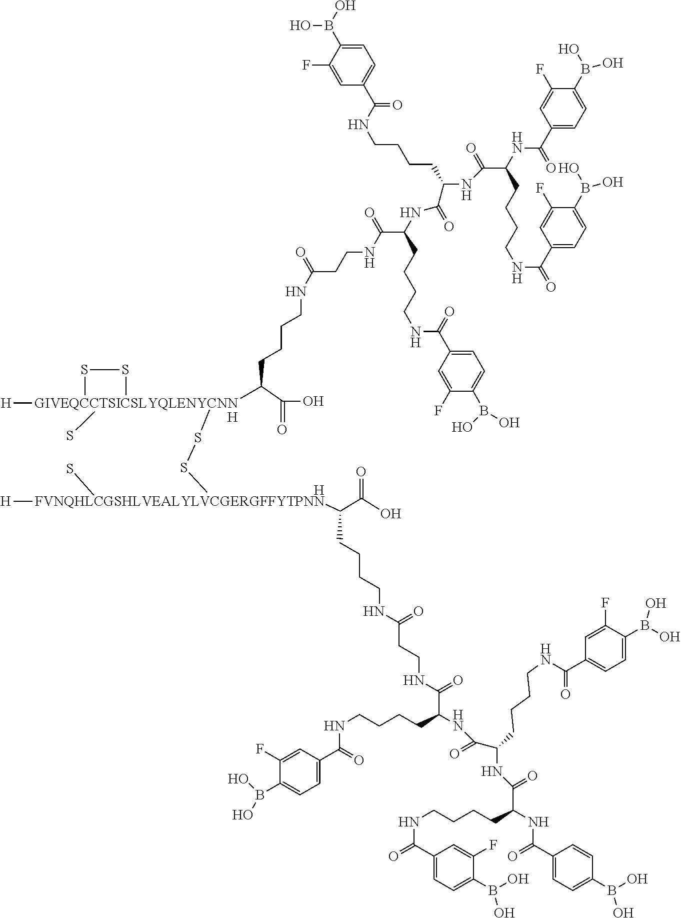

Example 125 was made similarly to example 101 from A22K desB30 human insulin and building block of example 14.

### Example 126

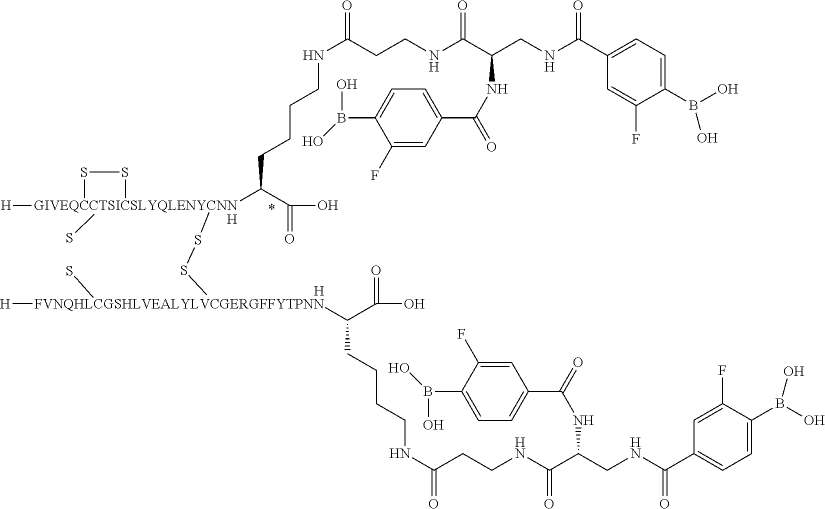

Example 126 was made similarly to example 101 from A22K desB30 human insulin and building block of example 15.

### Example 127

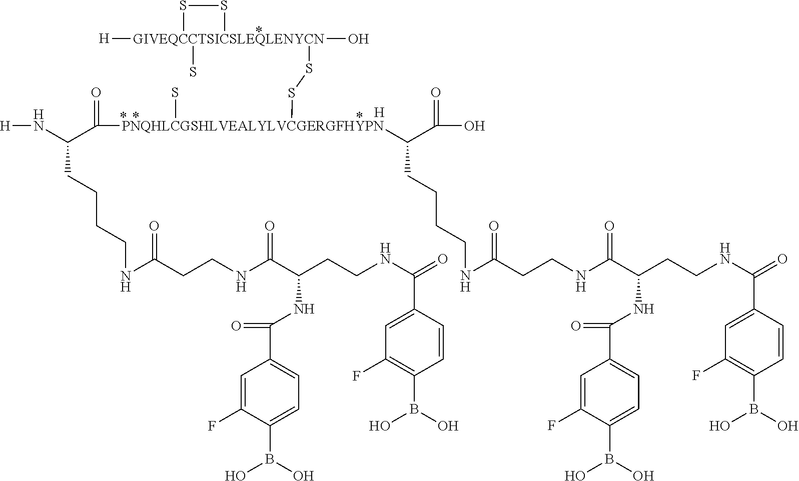

Example 127 was made similarly to example 101 from A14E B1K B2P B25H desB27 desB30 human insulin by first acylating insulin with gamma-aminobutyric acid, followed by building block of example 15.

### Example 128

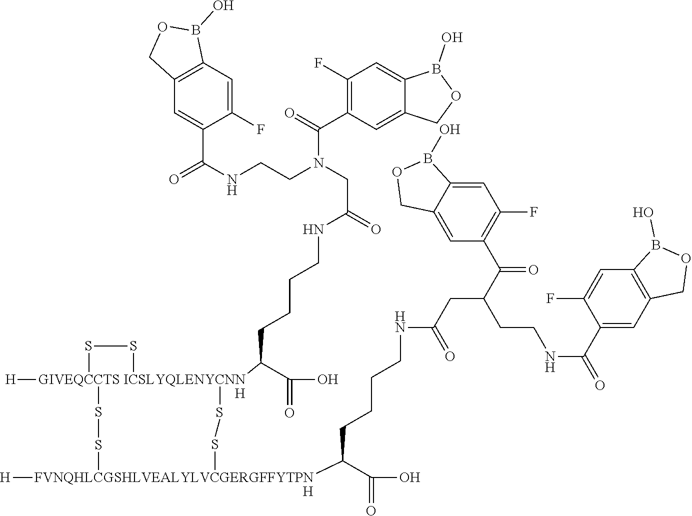

Example 128 was made similarly to example 101 from A22K desB30 human insulin and building block of example 15.

### Example 129

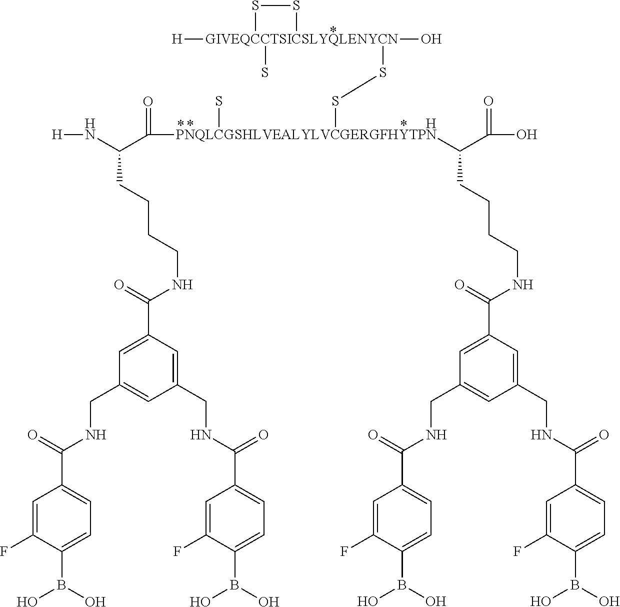

Example 129 was made similarly to example 101 from A14E B1K B2P B25H desB27 desB30 human insulin and building block of example 3.

### Example 130

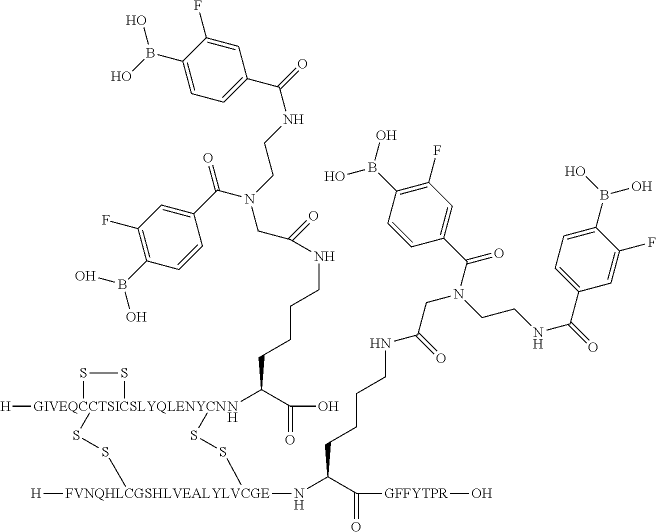

Example 130 was made similarly to example 101 from A22K B22K B29R desB30 human insulin and building block of example 9.

### Example 131

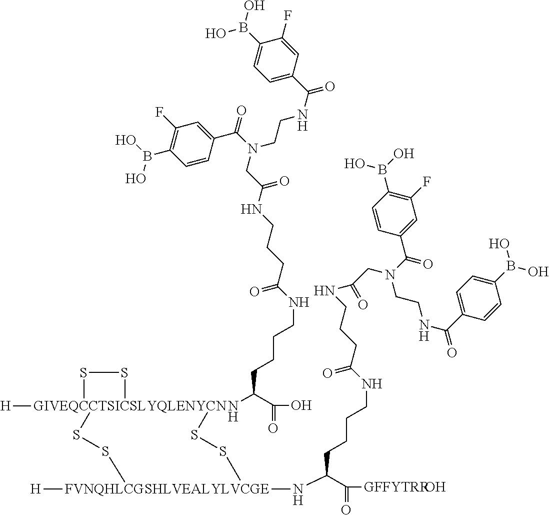

Example 131 was made similarly to example 101 from A22K B22K B29R desB30 human insulin by first acylating insulin with gamma-aminobutyric acid, followed by building block of example 9.

### Example 132

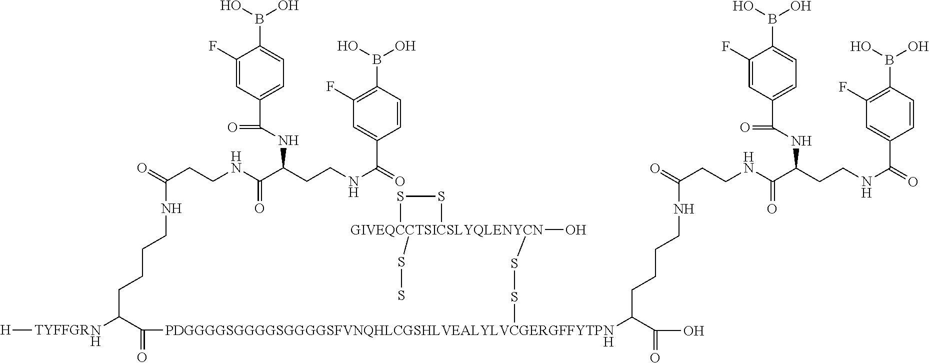

Example 132 was made similarly to example 101 from B1-TYFFGRKPDGGGGSGGGGSGGGGS desB30 human insulin by first acylating insulin with gamma-aminobutyric acid, followed by building block of example 15.

### Example 133

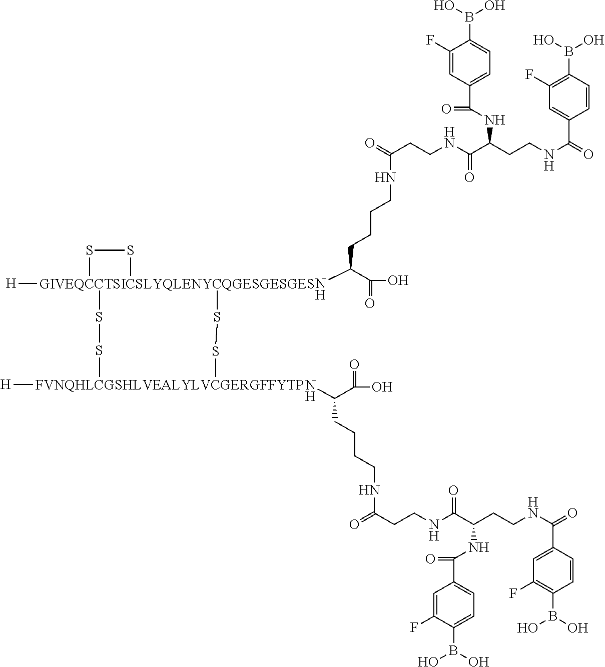

Example 133 was made similarly to example 101 from A21Q (GES)3K desB30 human insulin and building block of example 15.

### Example 134

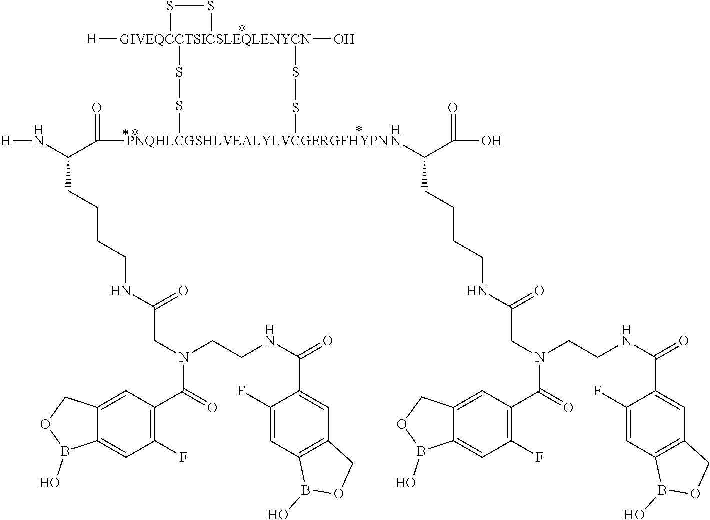

Example 134 was made similarly to example 101 from A14E B1K B2P B25H desB27 desB30 and building block of example 10.

### Example 135

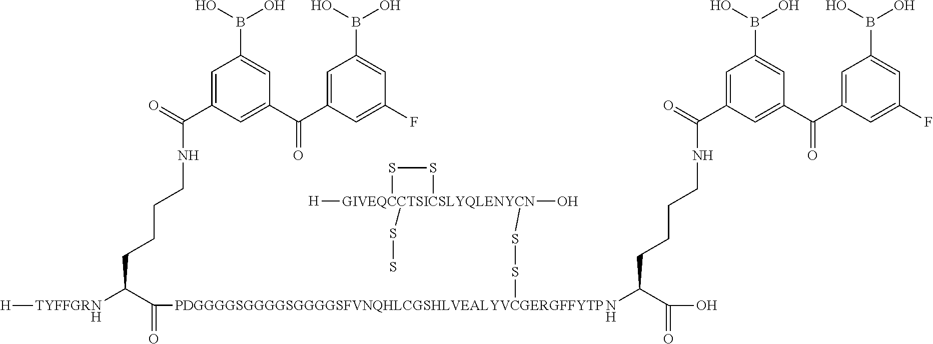

Example 135 was made similarly to example 101 from B1-TYFFGRKPDGGGGSGGGGSGGGGS desB30 human insulin and building block of example 16.

### Example 136

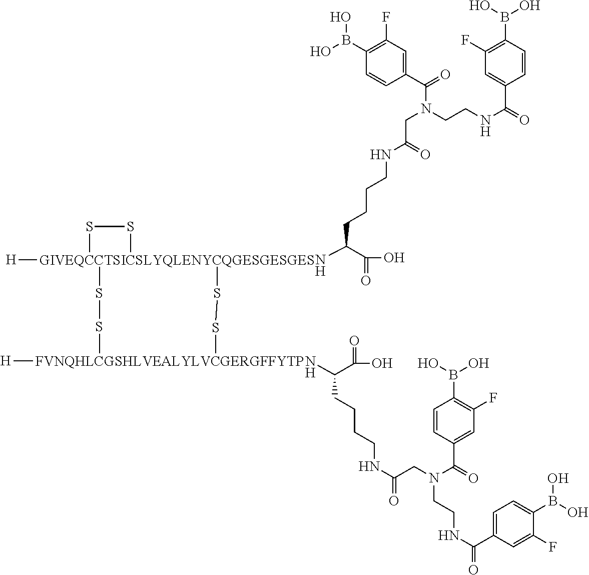

Example 136 was made similarly to example 101 from A21Q (GES)3K desB30 human insulin and building block of example 9.

### Example 137

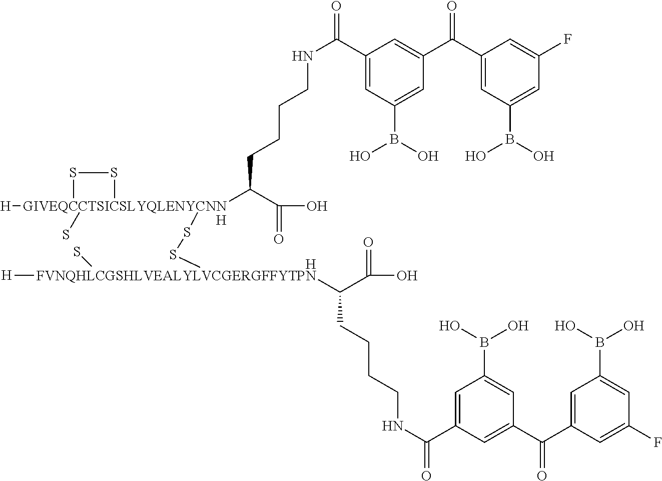

Example 137 was made similarly to example 101 from A22K desB30 human insulin and building block of example 16.

### Example 138

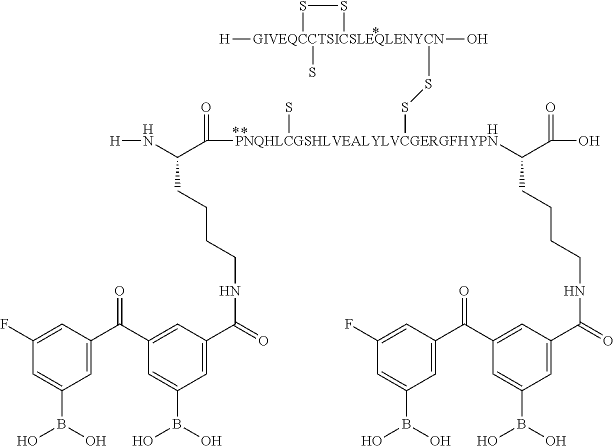

Example 138 was made similarly to example 101 from A14E B1K B2P B25H desB27 desB30 human insulin and building block of example 16.

### Example 139

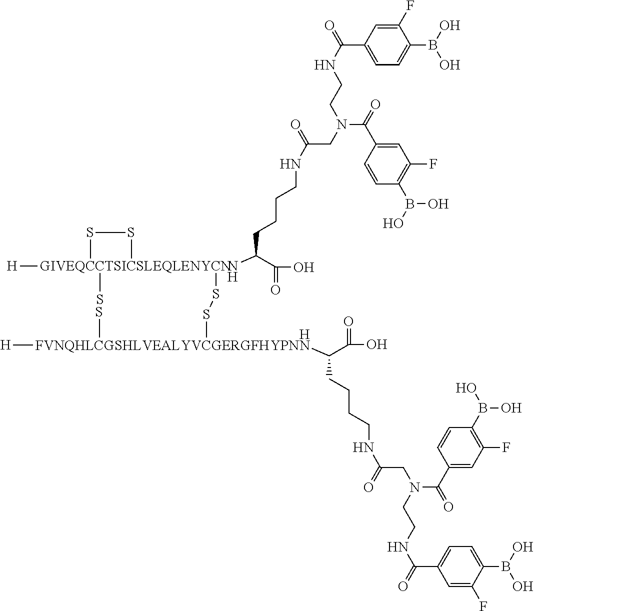

Example 139 was made similarly to example 101 from A14E A22K B25H desB27 desB30 human insulin and building block of example 9.

### Example 140

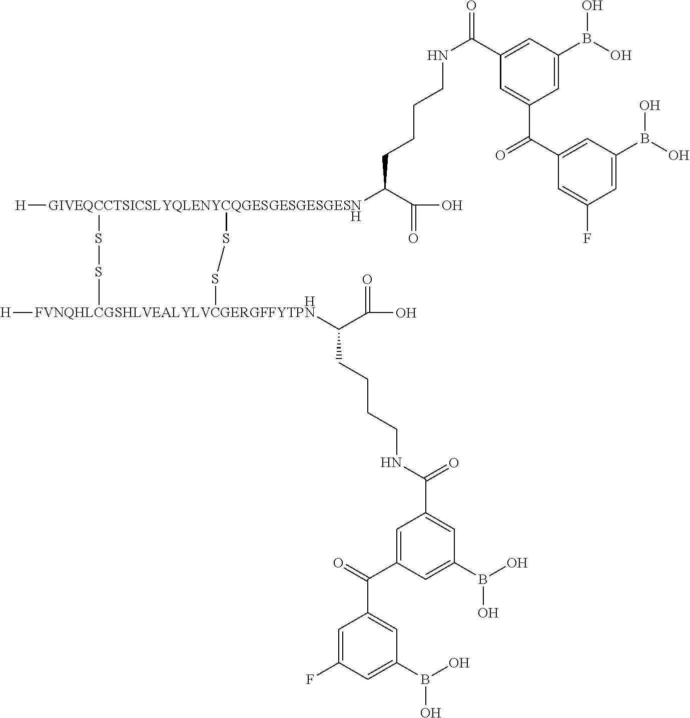

Example 140 was made similarly to example 101 from A21Q (GES)3K desB30 human insulin and building block of example 16.

### Example 141

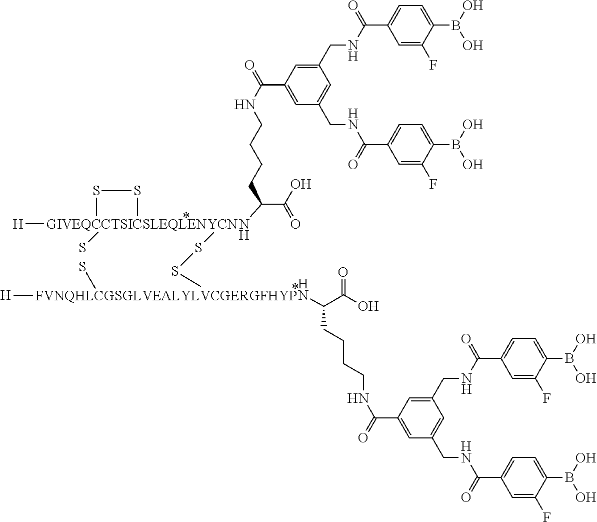

Example 141 was made similarly to example 101 from A14E A22K B25H desB27 desB30 human insulin and building block of example 2.

### Example 142

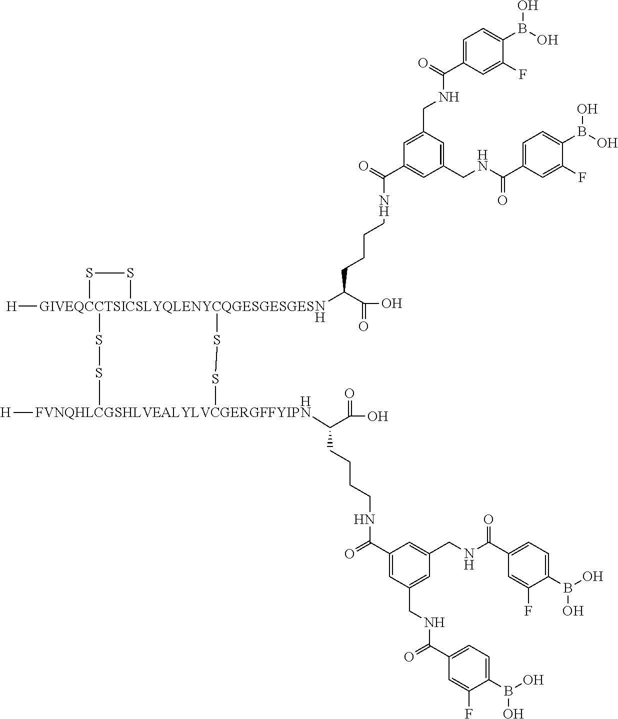

Example 142 was made similarly to example 101 from A21Q (GES)3K desB30 human insulin and building block of example 2.

### Example 143

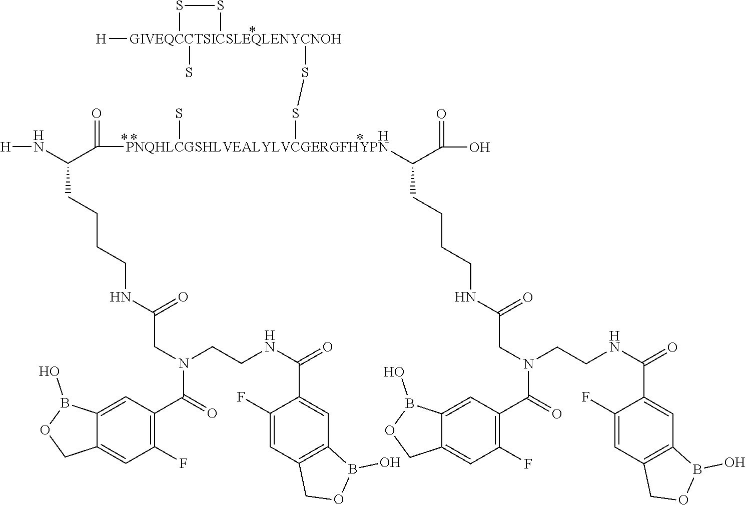

Example 143 was made similarly to example 101 from A14E B1K B2P B25H desB27 desB30 human insulin and building block of example 10.

### Example 144

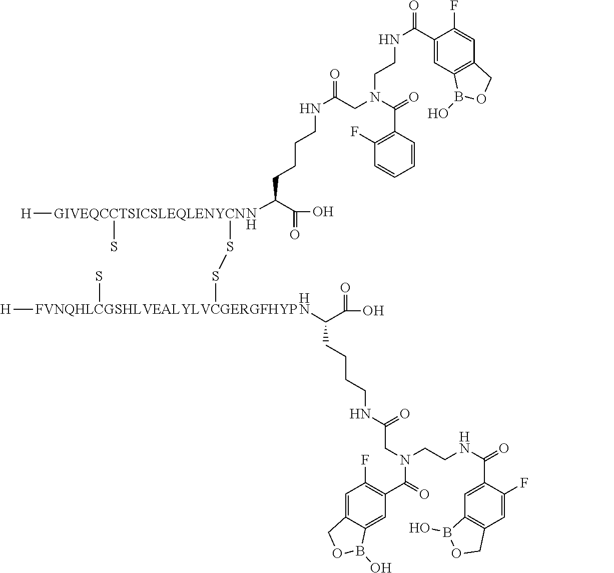

Example 144 was made similarly to example 101 from A14E A22K B25H desB27 desB30 human insulin and building block of example 10.

### Example 145

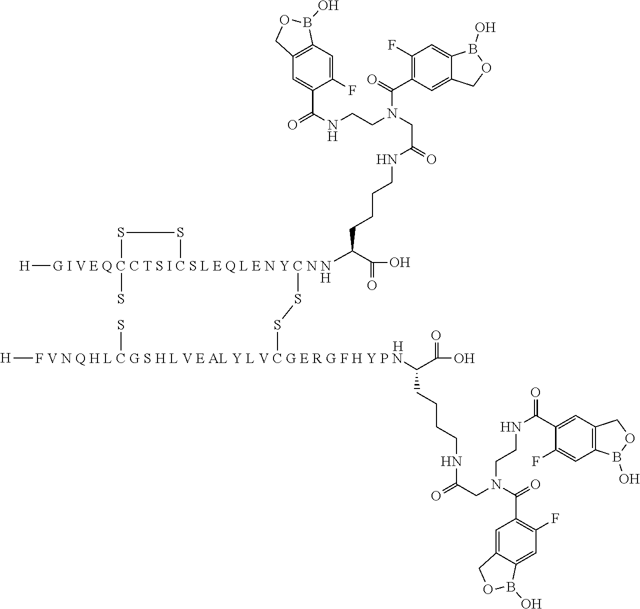

Example 145 was made similarly to example 101 from A14E A22K B25H desB27 desB30 human insulin and building block of example 10.

Example 146:

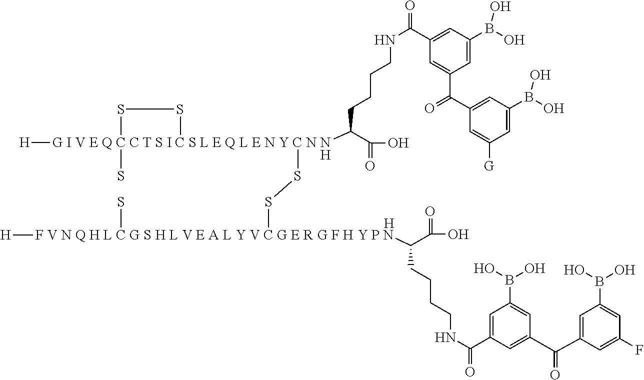

Example 146 was made similarly to example 101 from A14E A22K B25H desB27 desB30 human insulin and building block of example 16.

### Example 147

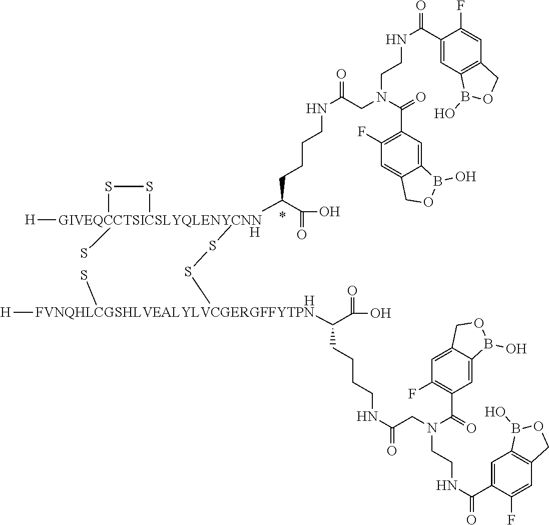

Example 147 was made similarly to example 101 from A22K desB30 human insulin and building block of example 10.

### Example 148

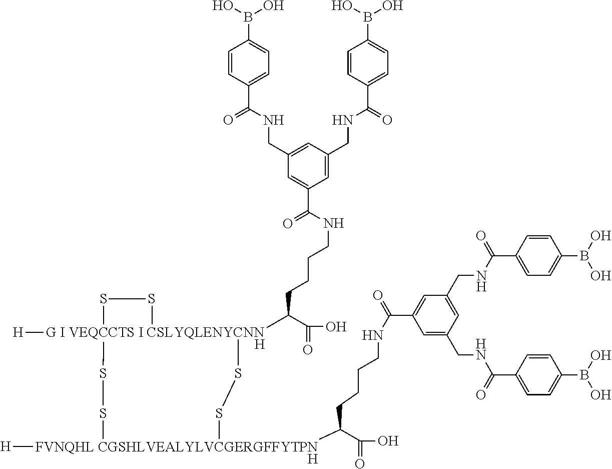

Example 148 was made similarly to example 101 from A22K desB30 human insulin and building block of example 17.

### Example 149

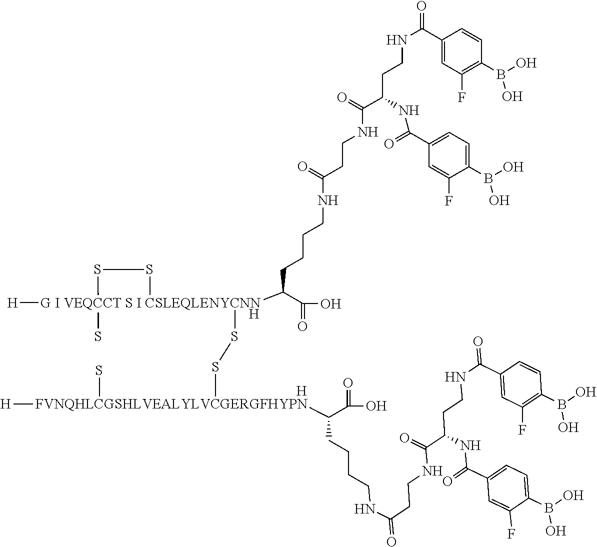

Example 149 was made similarly to example 101 from A14E A22K B25H desB27 desB30 human insulin and building block of example 15.

### Example 150

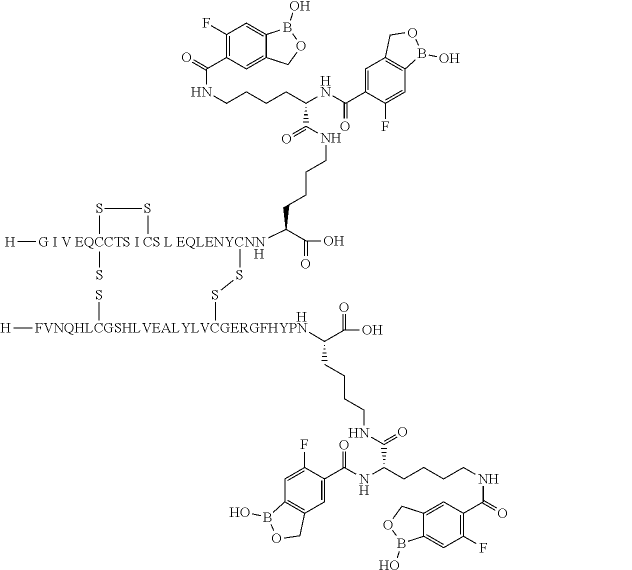

Example 150 was made similarly to example 101 from A14E A22K B25H desB27 desB30 human insulin and building block of example 18.

### Example 151

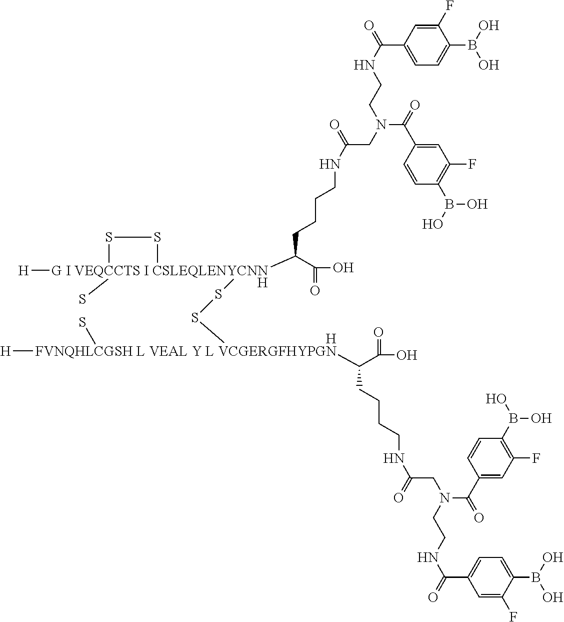

Example 151 was made similarly to example 101 from A14E A22K B25H B27P B28G desB30 human insulin and building block of example 9.

### Example 152

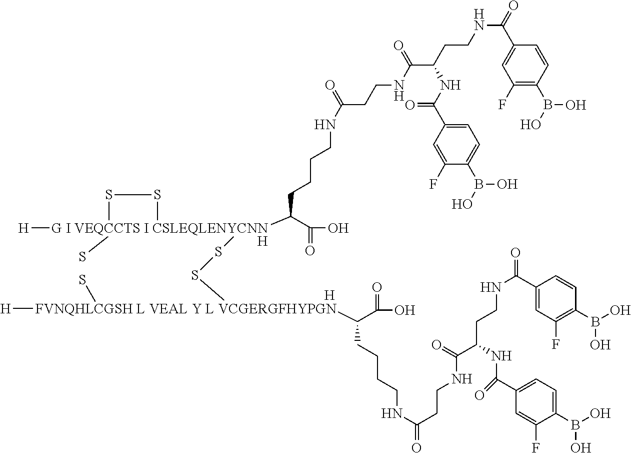

Example 152 was made similarly to example 101 from A14E A22K B25H B27P B28G desB30 human insulin and building block of example 15.

### Example 153

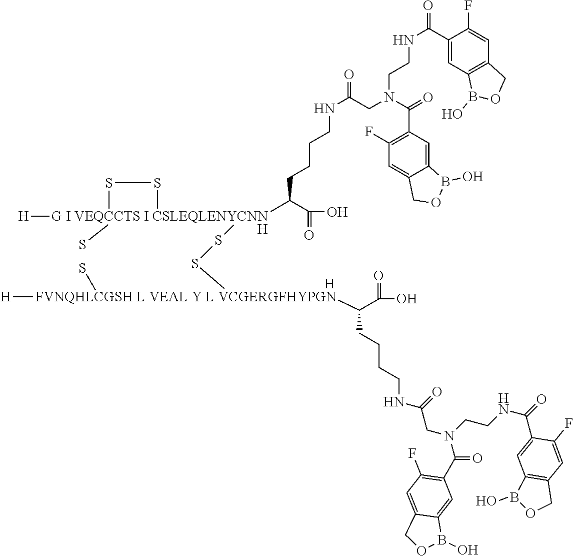

Example 153 was made similarly to example 101 from A14E A22K B25H B27P B28G desB30 human insulin and building block of example 9.

### Example 154

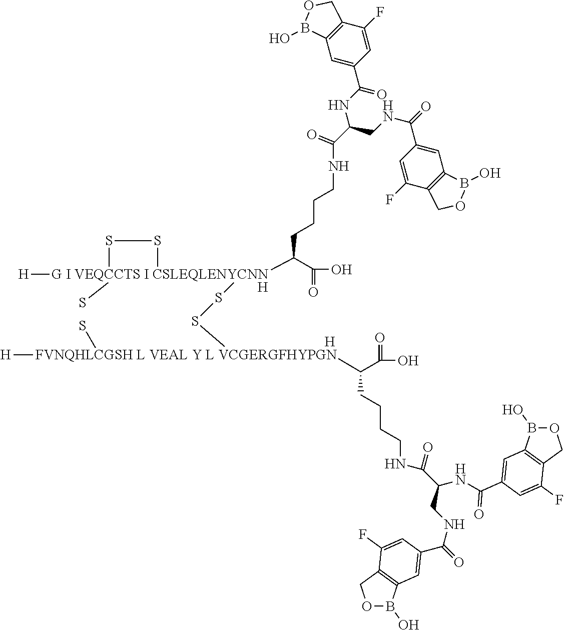

Example 154 was made similarly to example 101 from A14E A22K B25H B27P B28G desB30 human insulin and building block of example 19.

### Example 155

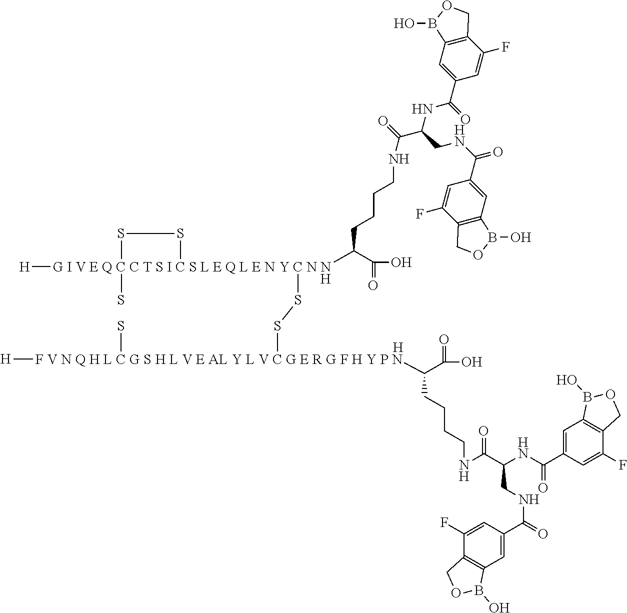

Example 155 was made similarly to example 101 from A14E A22K B25H desB27 desB30 human insulin and building block of example 19.

### Example 156

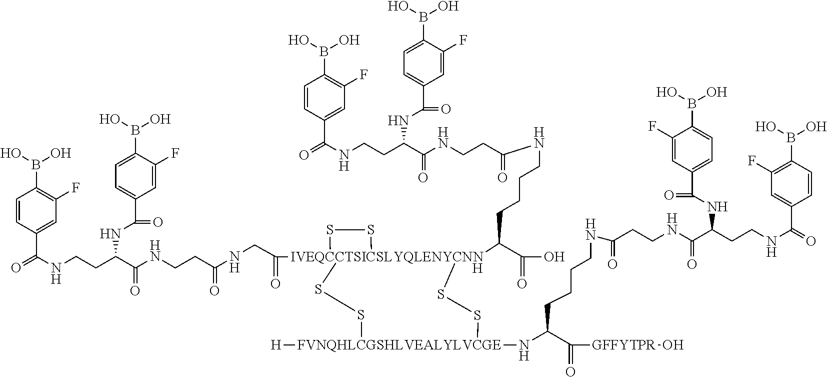

Example 156 was made similarly to example 103 from A22K B22K B29R desB30 human insulin and building block of example 15.

### Example 157

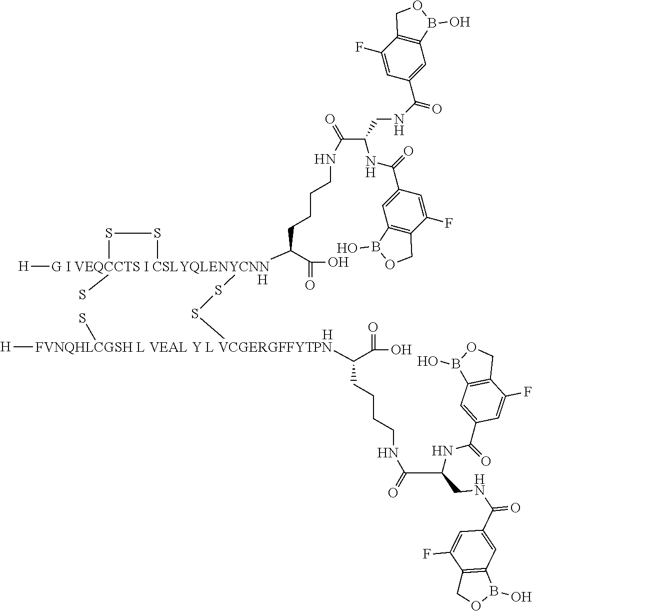

Example 157 was made similarly to example 101 from A22K desB30 human insulin and building block of example 19.

### Example 158

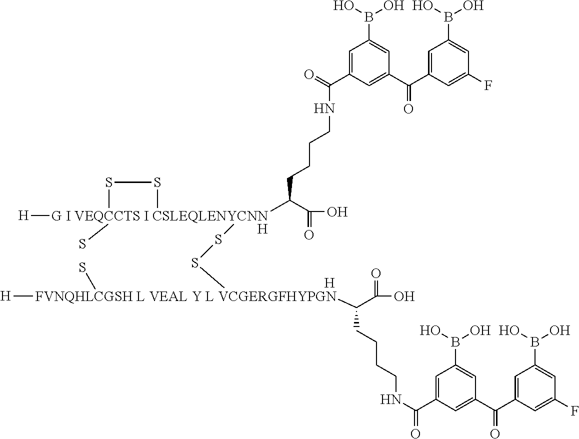

Example 158 was made similarly to example 101 from A14E A22K B25H B27P B28G desB30 human insulin and building block of example 16.

### Example 159

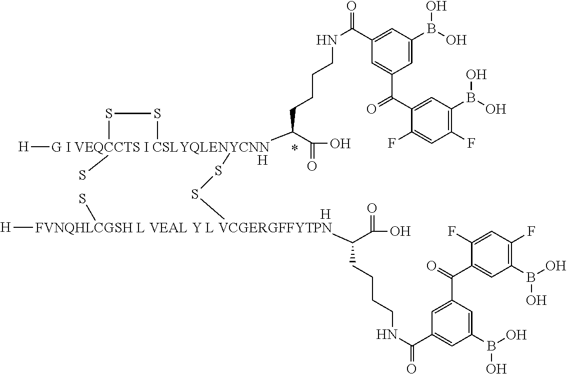

Example 159 was made similarly to example 101 from A22K desB30 human insulin and building block of example 20.

### Example 160

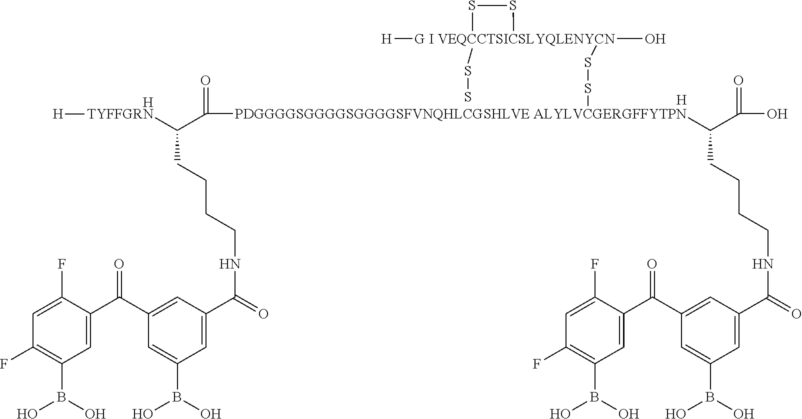

Example 160 was made similarly to example 101 from B1-TYFFGRKPDGGGGSGGGGSGGGGS desB30 human insulin and building block of example 20.

### Example 161

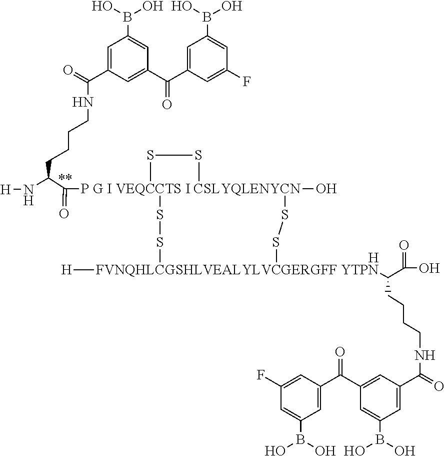

Example 161 was made similarly to example 101 from A-2K A-1P desB30 human insulin and building block of example 16.

### Example 162

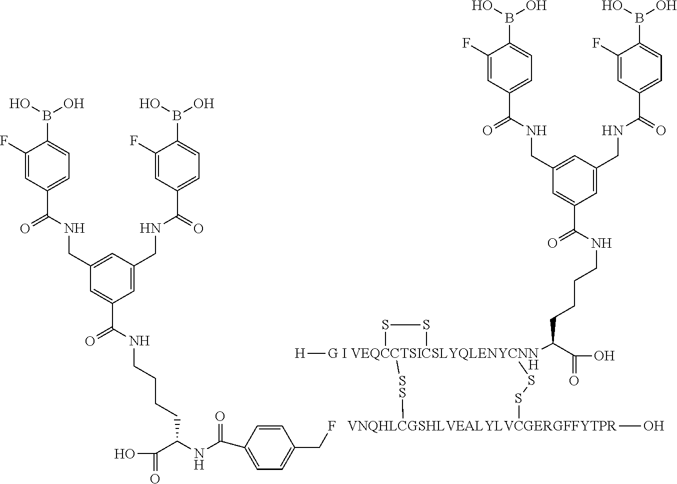

Example 162 was made similarly to example 105 from A22K B29R desB30 human insulin and building block of example 2.

### Example 163

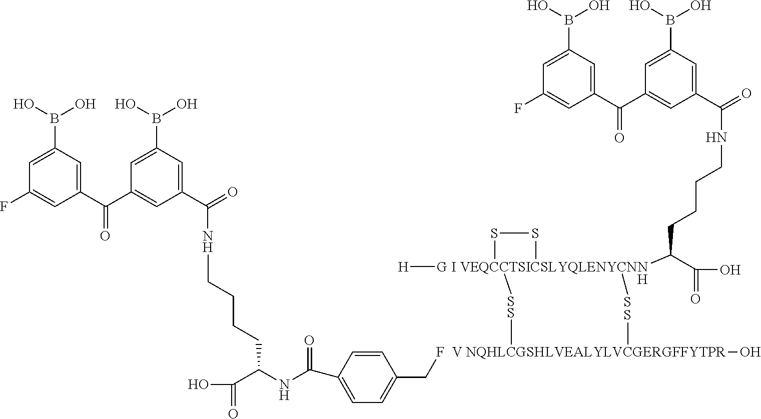

Example 163 was made similarly to example 105 from A22K B29R desB30 human insulin and building block of example 16.

### Example 164

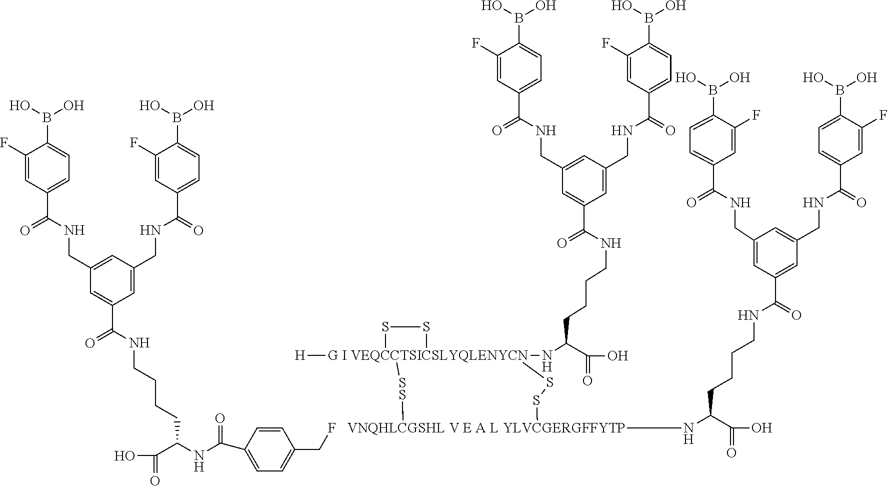

Example 164 was made similarly to example 105 from A22K desB30 human insulin and building block of example 2.

### Example 165

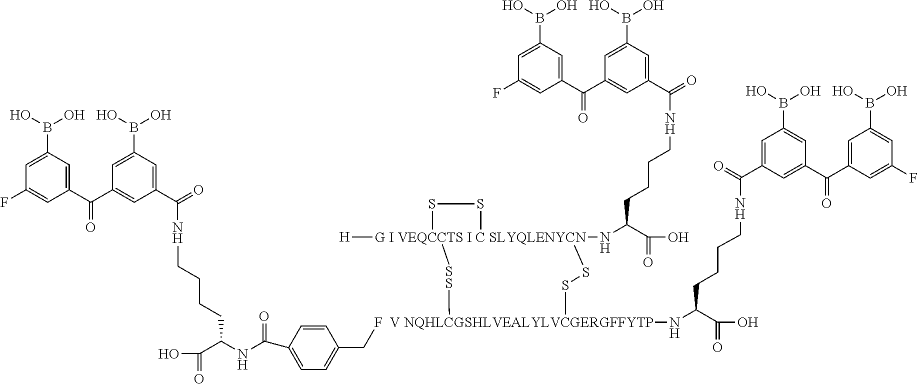

Example 165 was made similarly to example 105 from A22K desB30 human insulin and building block of example 16.

### Example 166

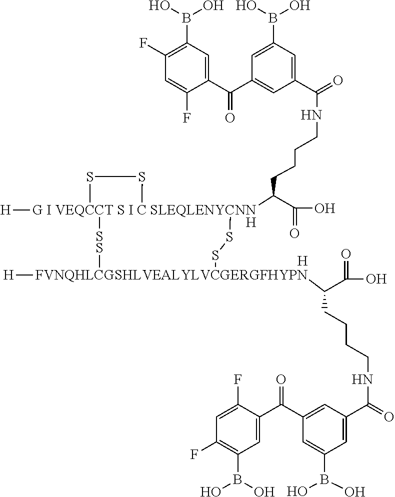

Example 166 was made similarly to example 101 from A14E A22K B25H desB27 desB30 and building block of example 20.

### Example 167

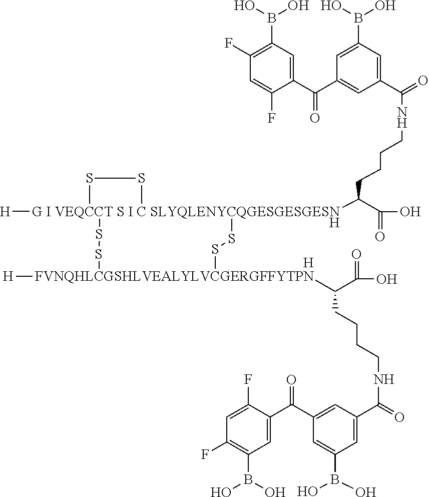

Example 167 was made similarly to example 101 from A21Q (GES)3K desB30 human insulin and building block of example 20.

### Example 168

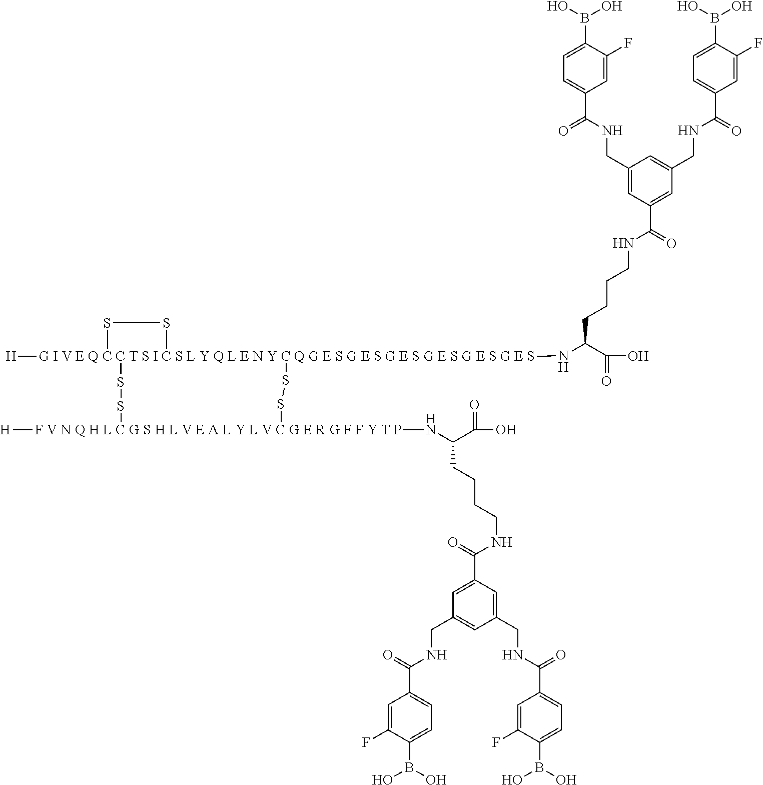

Example 168 was made similarly to example 101 from A21Q (GES)6K desB30 human insulin and building block of example 2.

### Example 169

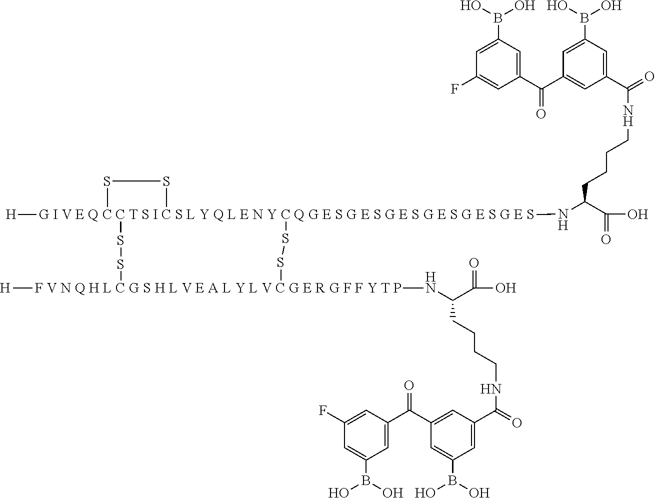

Example 169 was made similarly to example 101 from A21Q (GES)6K desB30 human insulin and building block of example 16.

### Example 170

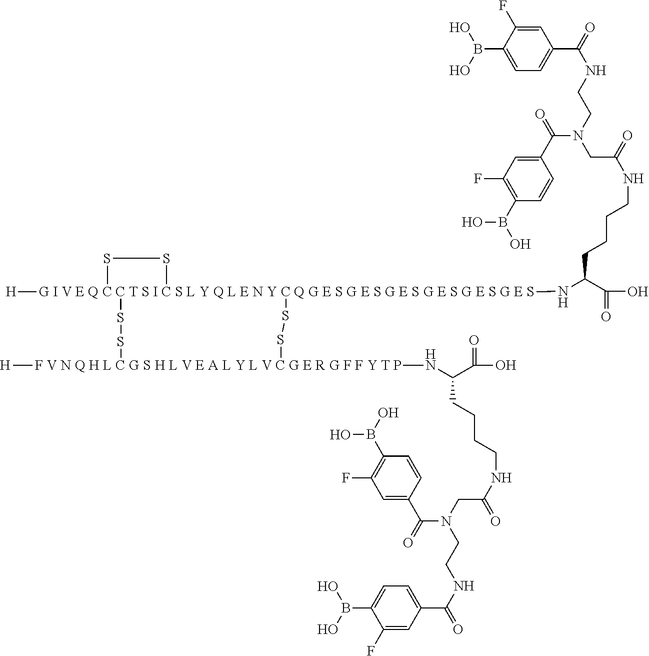

Example 170 was made similarly to example 101 from A21Q (GES)6K desB30 human insulin and building block of example 9.

### Example 171

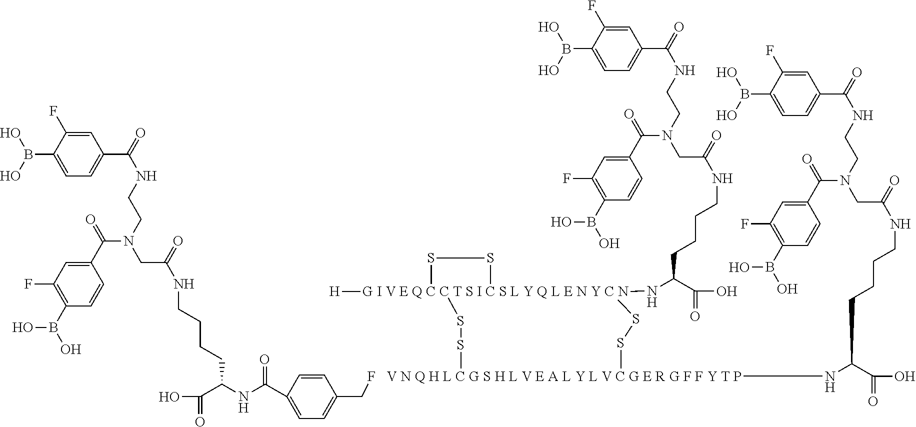

Example 171 was made similarly to example 105 from A22K desB30 human insulin and building block of example 9.

### Example 172

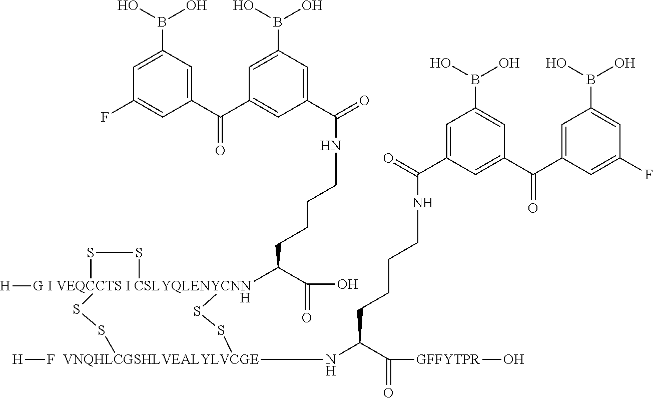

Example 172 was made similarly to example 101 from A22K B22K B29R desB30 human insulin and building block of example 16.

### Example 173

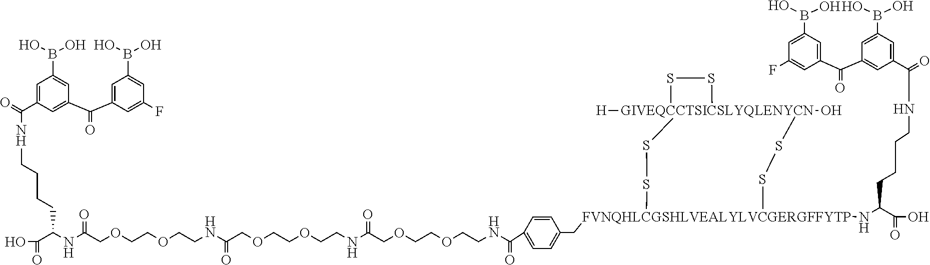

Example 173 was made similarly to example 105 from desB30 human insulin and building block of example 16.

### Example 174

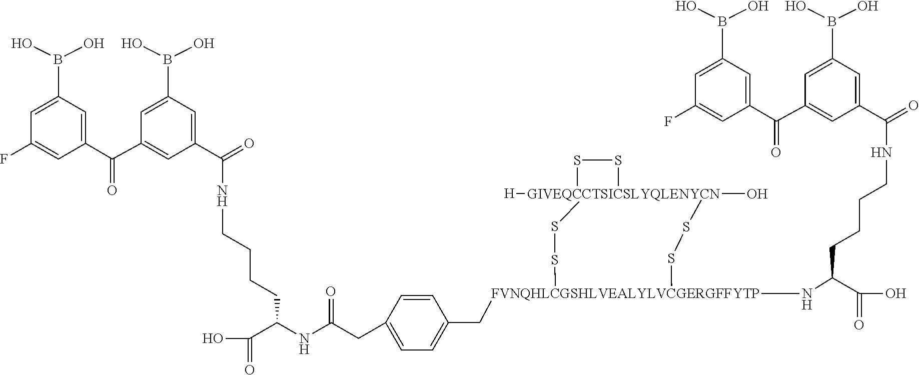

Example 174 was made similarly to example 105 from desB30 human insulin and building block of example 16.

### Example 175

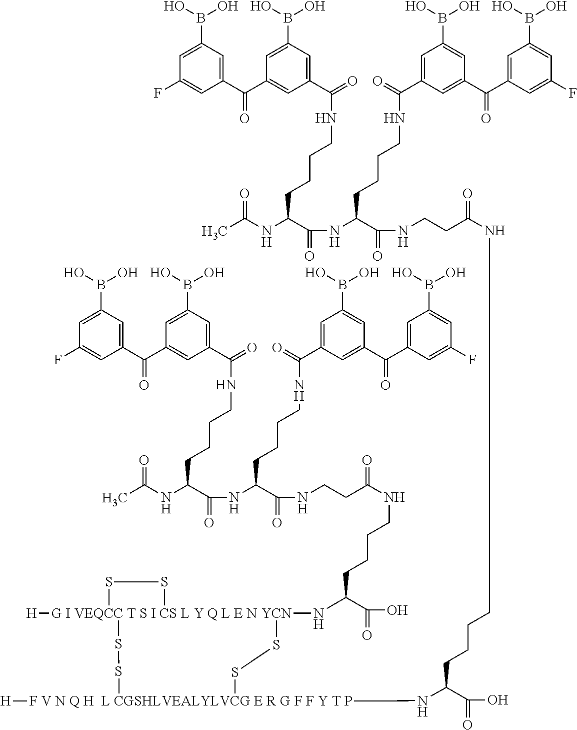

Example 175 was made similarly to example 101 from A22K desB30 human insulin and building block of example 16.

### Example 176

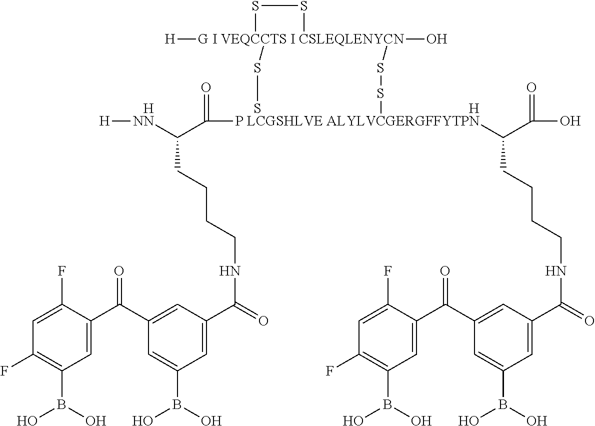

Example 176 was made similarly to example 101 from A14E desB1-B2 B4K B5P desB30 human insulin and building block of example 20.

### Example 177

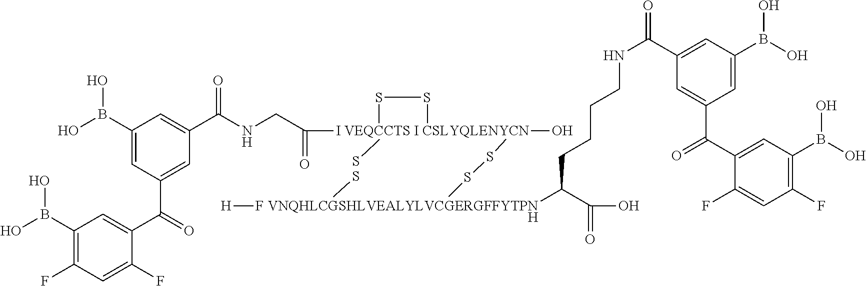

Example 177 was made similarly to example 103 from desB30 human insulin and building block of example 20.

### Example 178

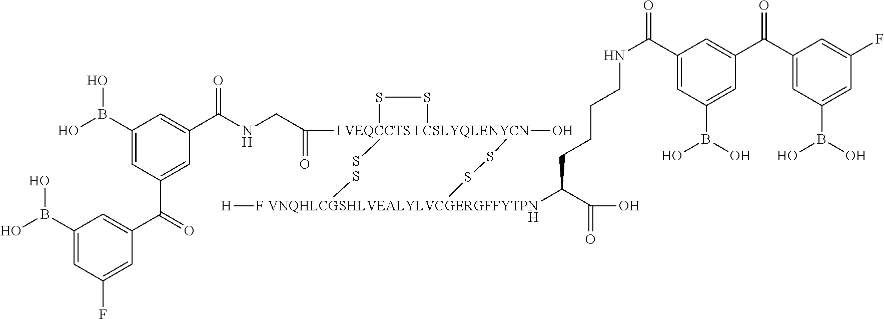

Example 178 was made similarly to example 103 from desB30 human insulin and building block of example 16.

### Example 179

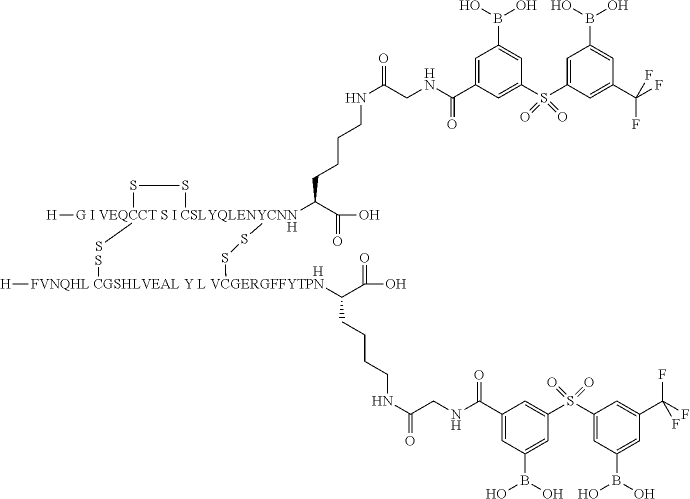

Example 179 was made similarly to example 101 from A22K desB30 human insulin and building block of example 22.

### Example 180

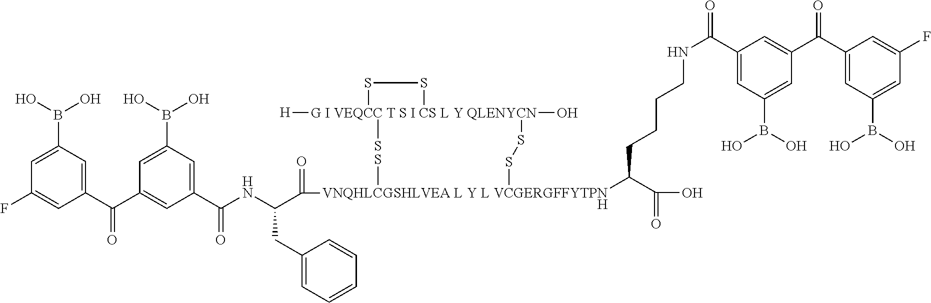

Example 180 was made similarly to example 101 from desB3d human insulin and building block of example 16.

### Example 181

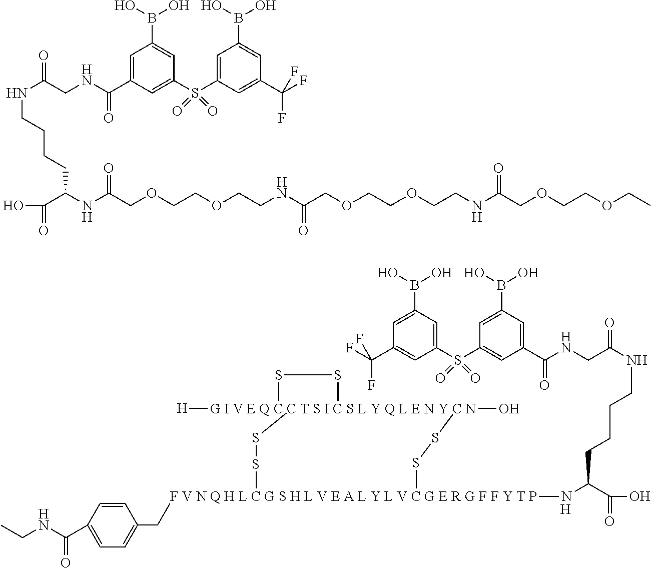

Example 181 was made similarly to example 101 from desB30 human insulin and building block of example 22.

### Example 182

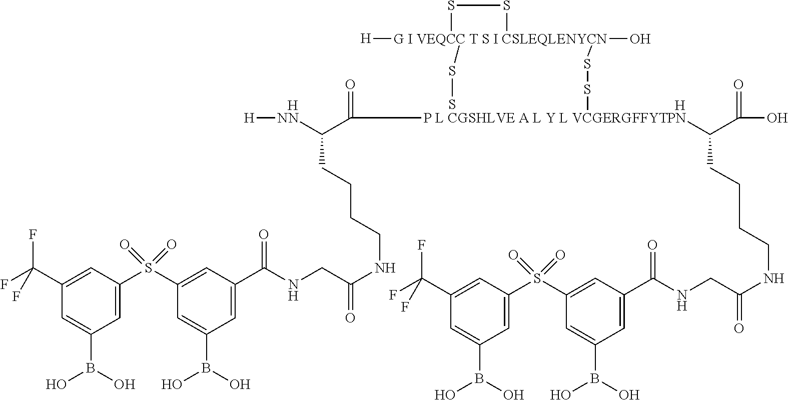

Example 182 was made similarly to example 101 from A14E desB1-B2 B4K B5P desB30 human insulin and building block of example 22.

### Example 183

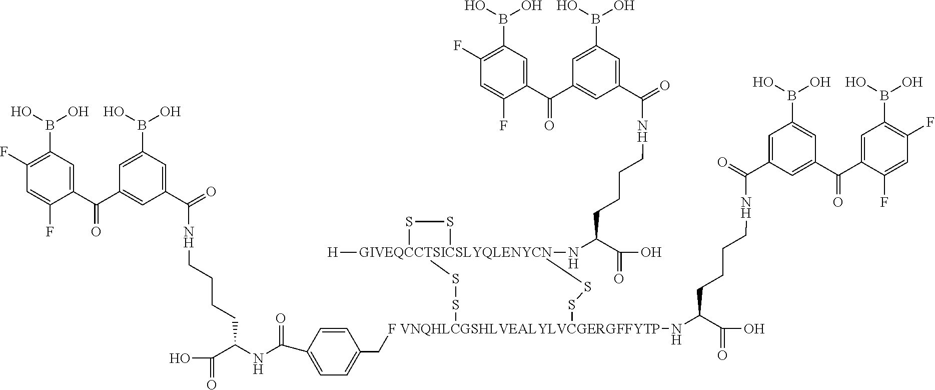

Example 183 was made similarly to example 105 from A22K desB30 human insulin and building block of example 20.

### Example 184

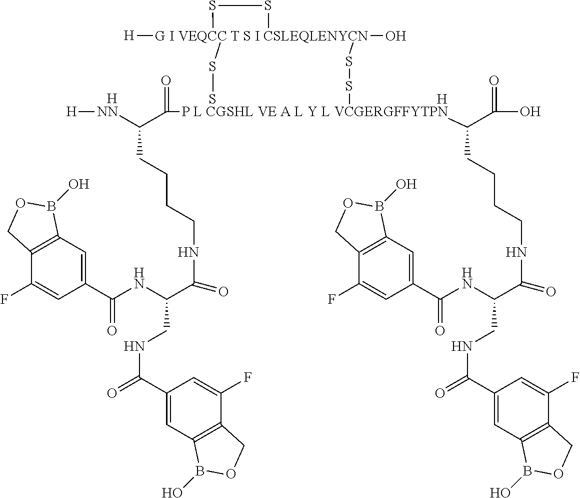

Example 184 was made similarly to example 101 from A14E desB1-B2 B4K B5P desB30 human insulin and building block of example 19.

### Example 185

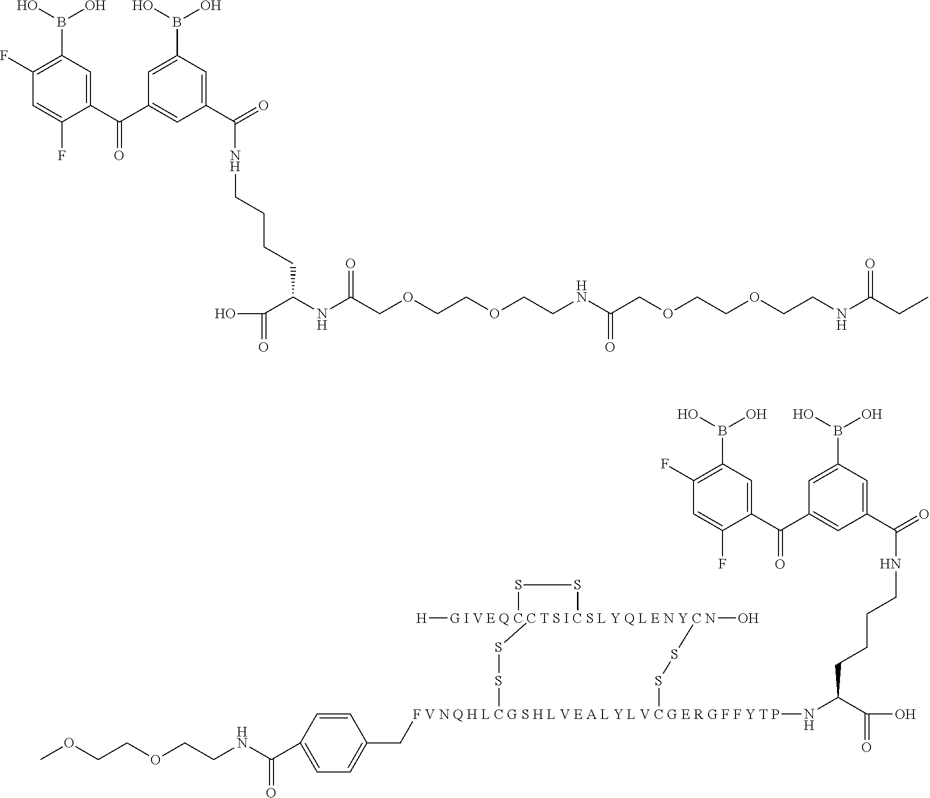

Example 185 was made similarly to example 105 from desB30 human insulin and building block of example 20.

### Example 186

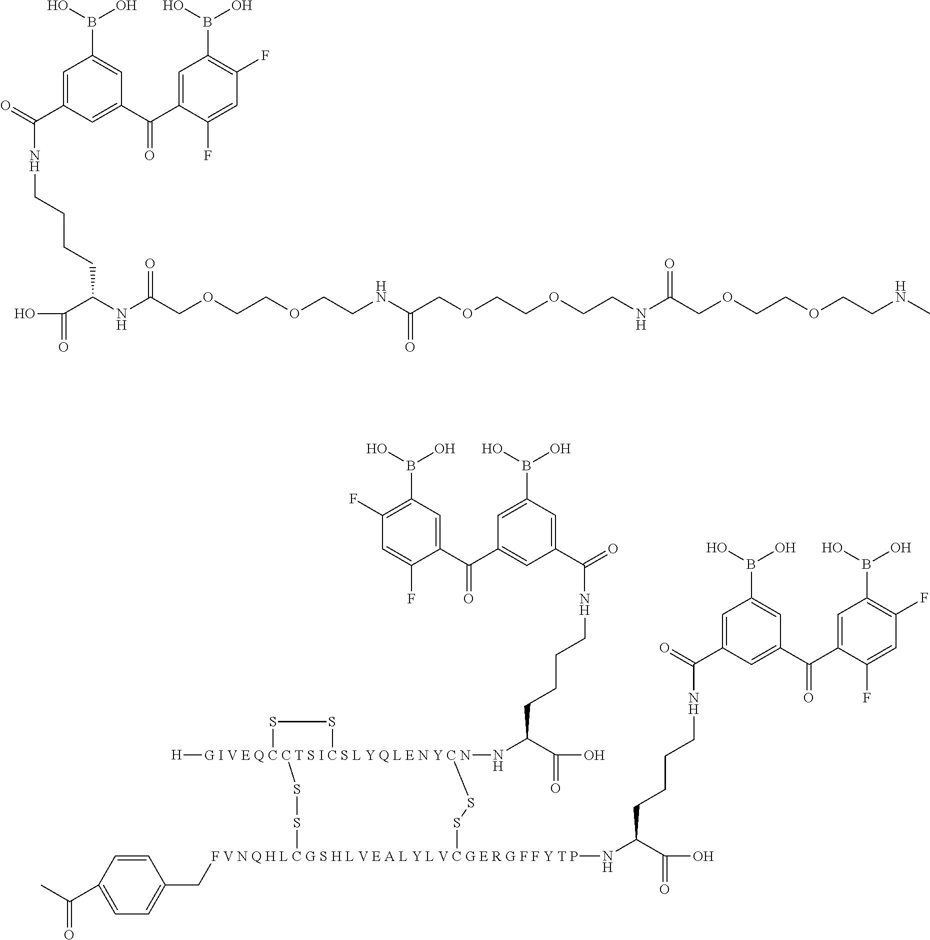

Example 186 was made similarly to example 105 from A22K desB30 human insulin and building block of example 20.

### Example 187

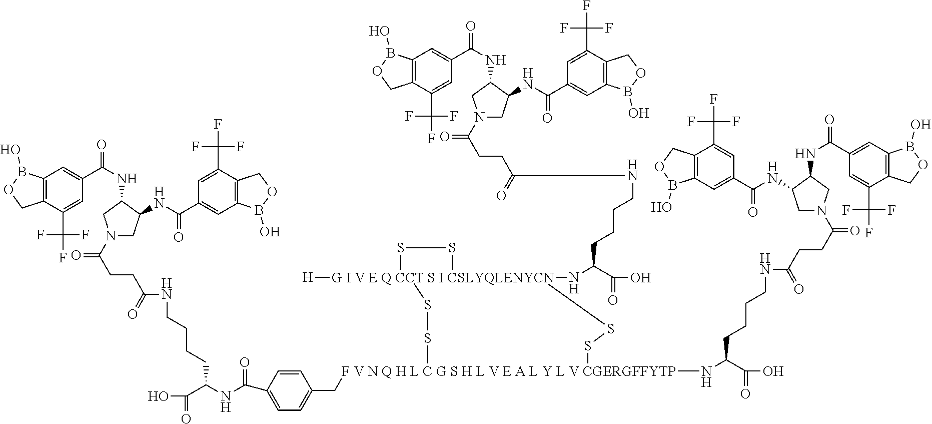

Example 187 was made similarly to example 105 from A22K desB30 human insulin and building block of example 24

### Example 188

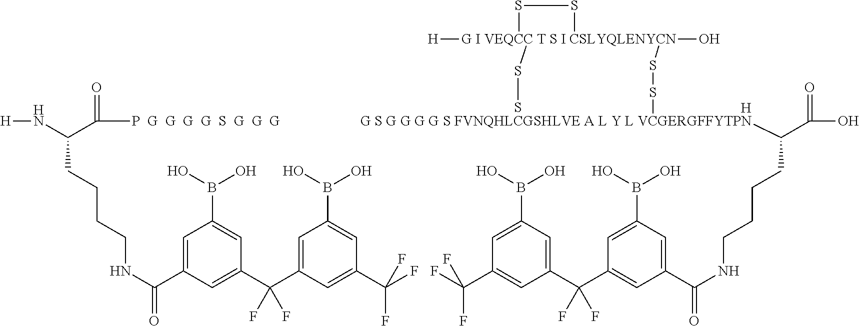

Example 188 was made similarly to example 101 from B1-KPGGGGSGGGGSGGGGS desB30 human insulin and building block of example 25.

### Example 189

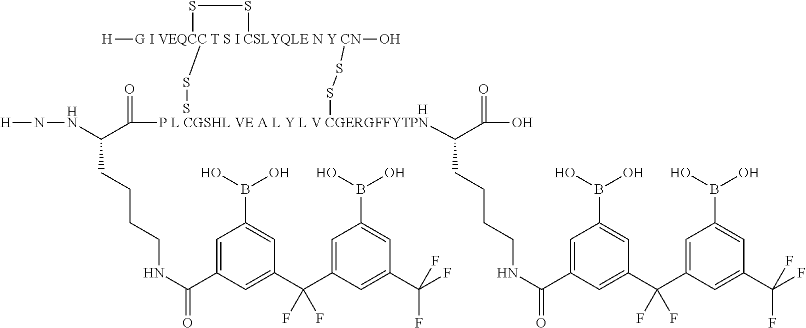

Example 189 was made similarly to example 101 from A14E desB1-B2 B4K B5P desB30 human insulin and building block of example 25.

### Example 190

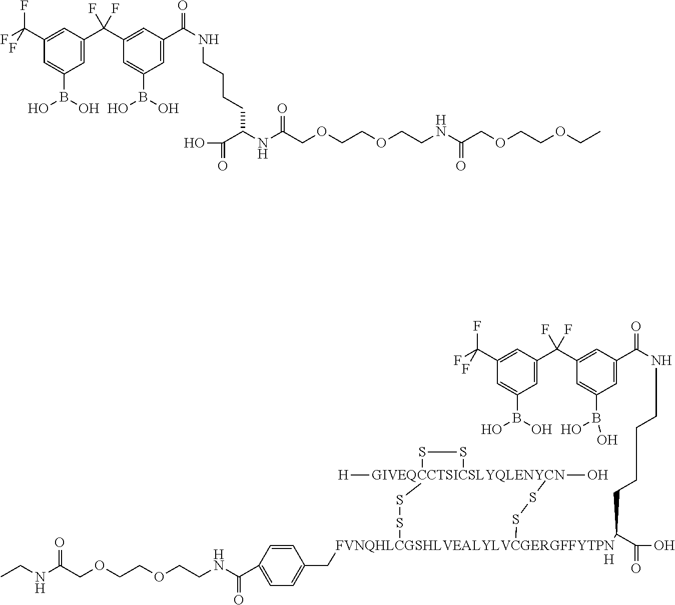

Example 190 was made similarly to example 105 from desB30 human insulin and building block of example 25.

### Example 191

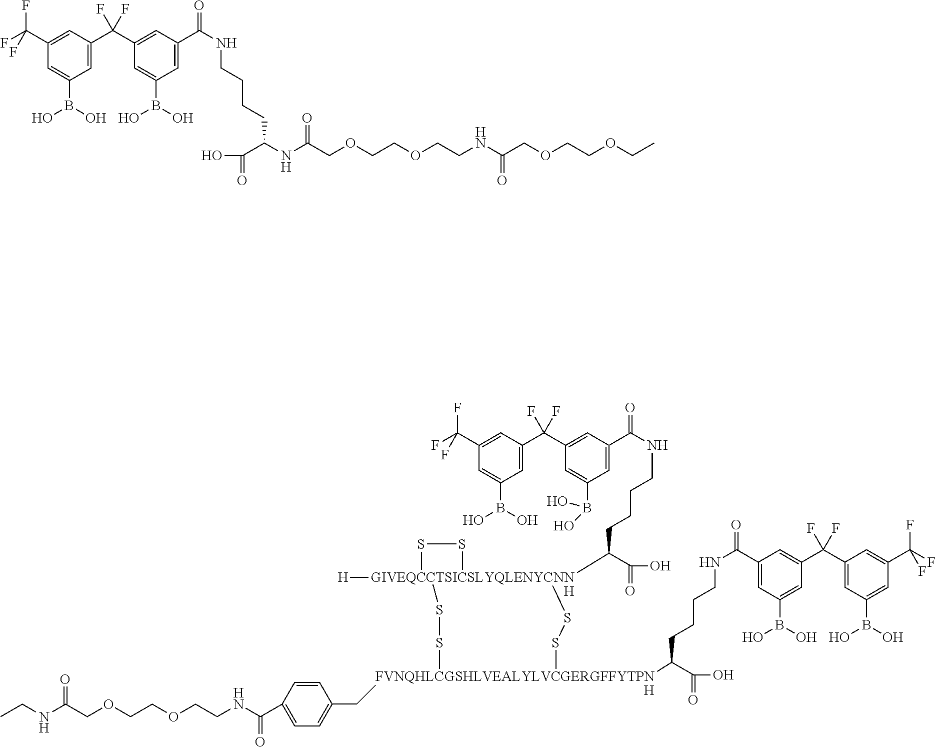

Example 191 was made similarly to example 105 from A22K desB30 human insulin and building block of example 25.

### Example 192

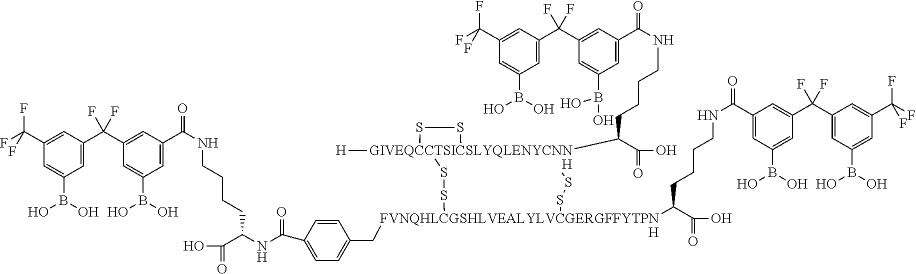

Example 192 was made similarly to example 105 from A22K desB30 human insulin and building block of example 25.

### Example 193

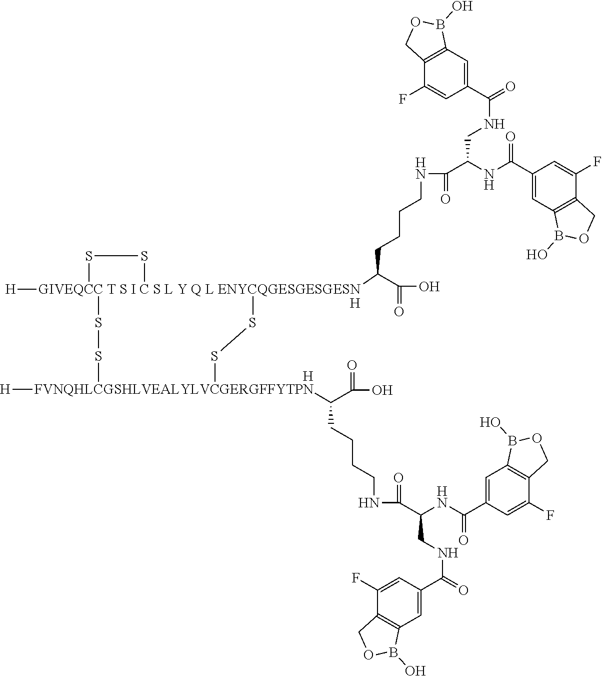

Example 193 was made similarly to example 101 from A21Q (GES)3K desB30 human insulin and building block of example 19.

### Example 194

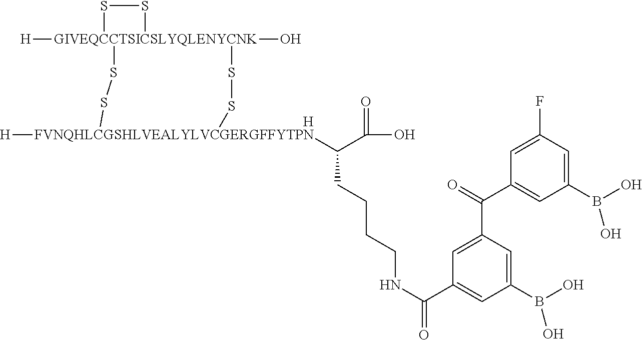

Example 194 was made similarly to example 101 from A22K desB30 human insulin and building block of example 16.

### Example 195

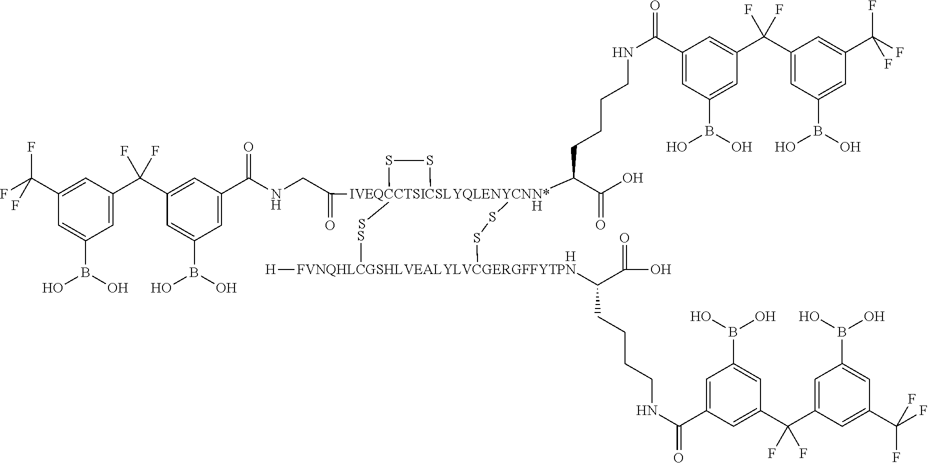

Example 195 was made similarly to example 101 from A22K desB30 human insulin and building block of example 25.

### Example 196

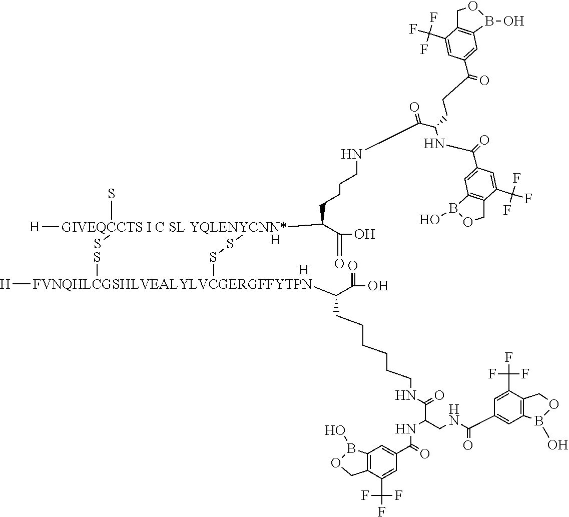

Example 196 was made similarly to example 101 from A22K desB30 human insulin and building block of example 26.

### Example 197

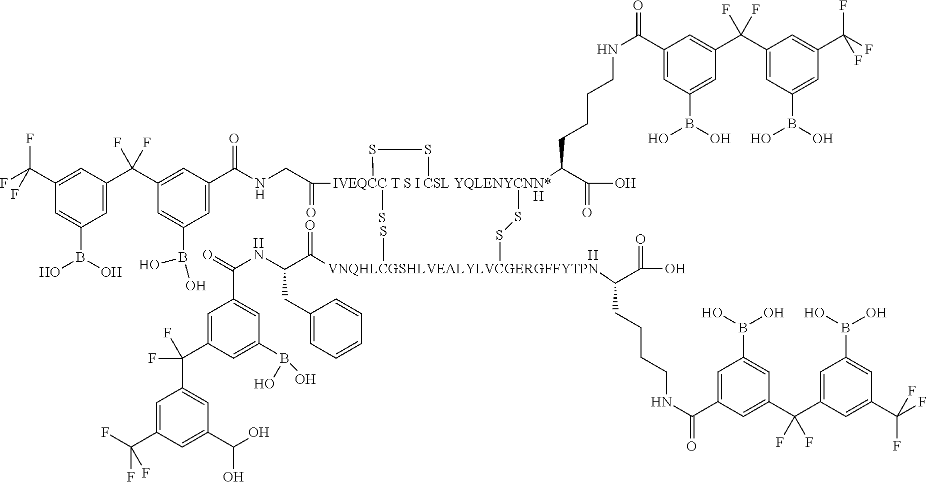

Example 197 was made similarly to example 101 from A22K desB30 human insulin and building block of example 25.

### Example 198

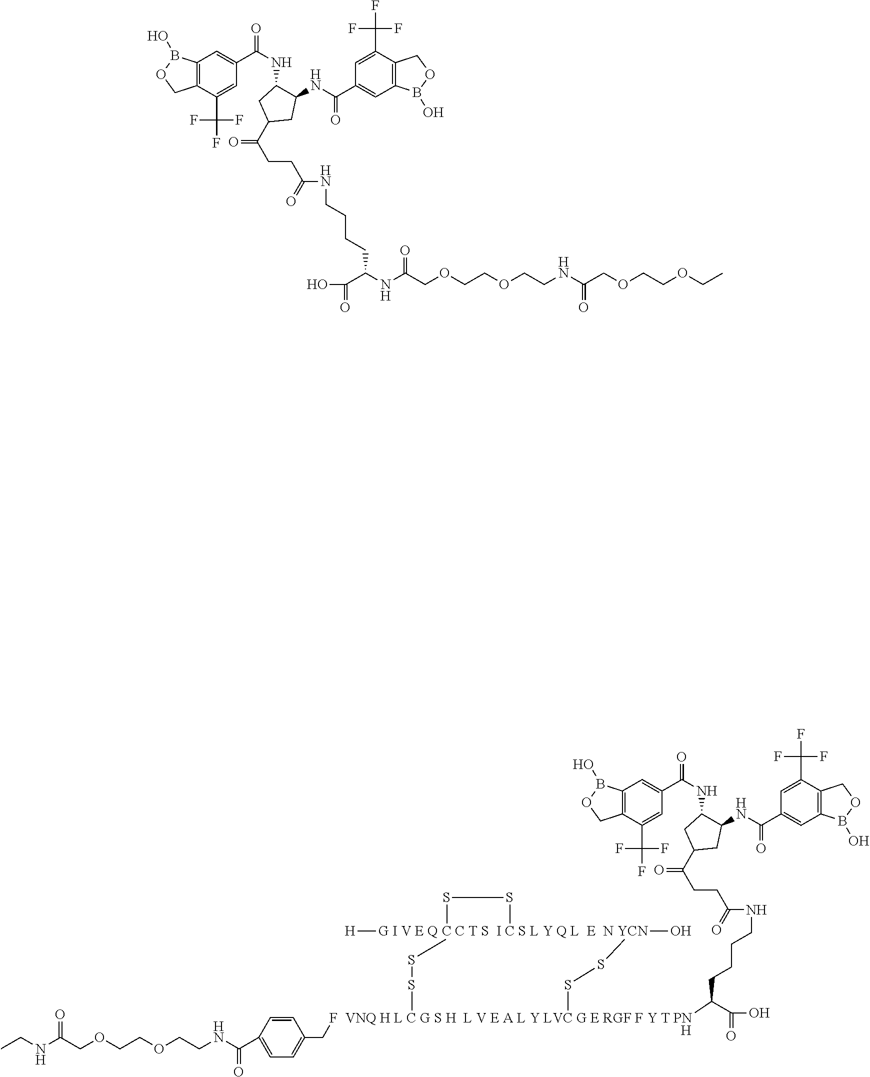

Example 198 was made similarly to example 105 from desB30 human insulin and building block of example 24.

### Example 199

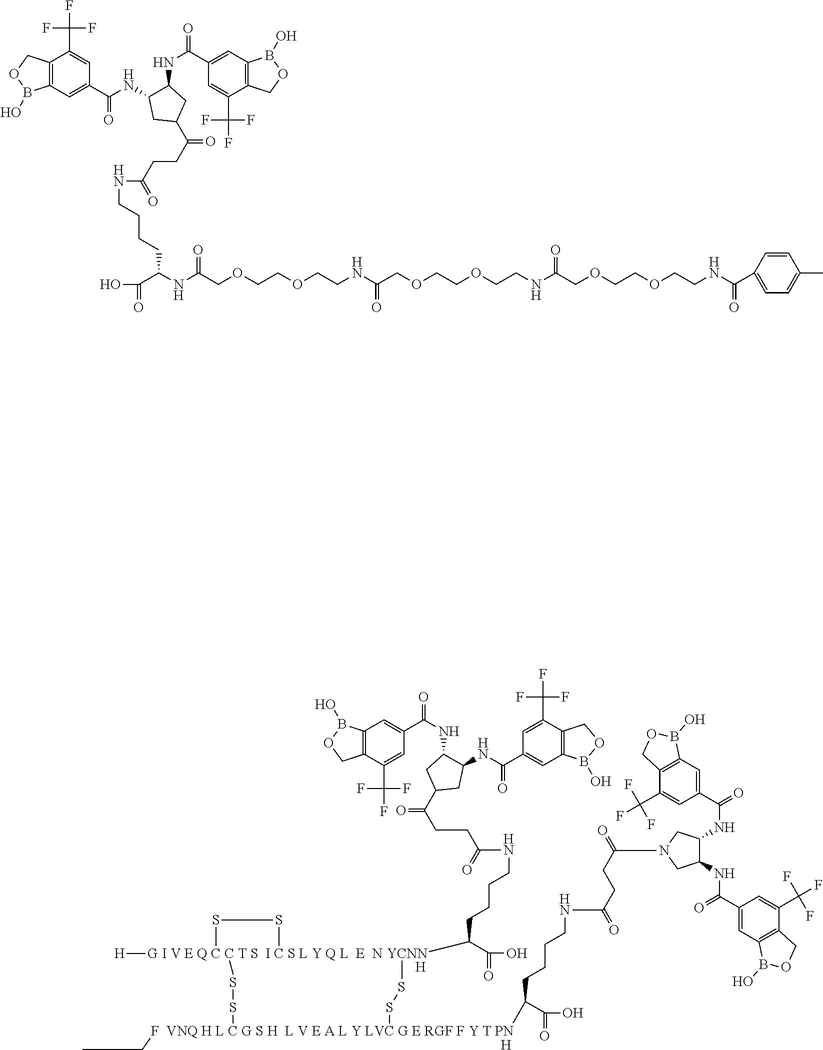

Example 199 was made similarly to example 105 from A22K desB30 human insulin and building block of example 24.

### Example 200

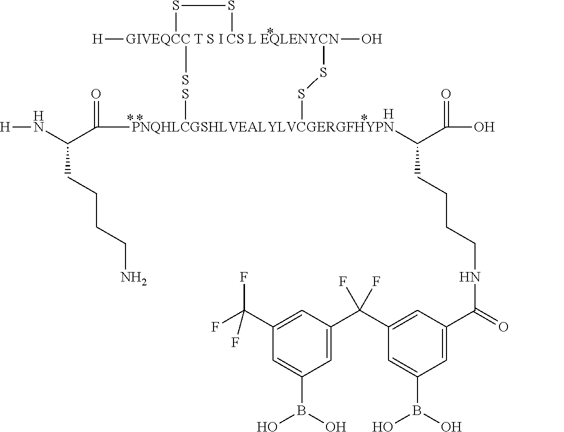

Example 200 was made similarly to example 101 from A14E B1K B2P B25H desB27 desB30 human insulin and building block of example 25.

### Example 201

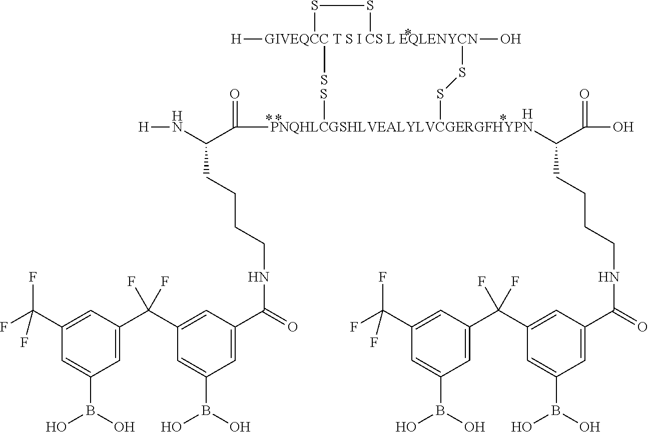

Example 201 was made similarly to example 101 from A14E B1K B2P B25H desB27 desB30 human insulin and building block of example 25.

### Example 202

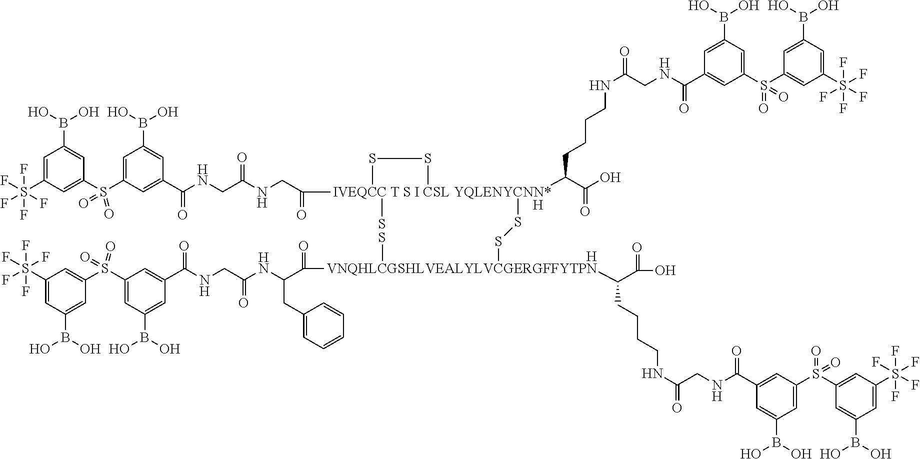

Example 202 was made similarly to example 101 from A22K desB30 human insulin and building block of example 27.

### Example 203

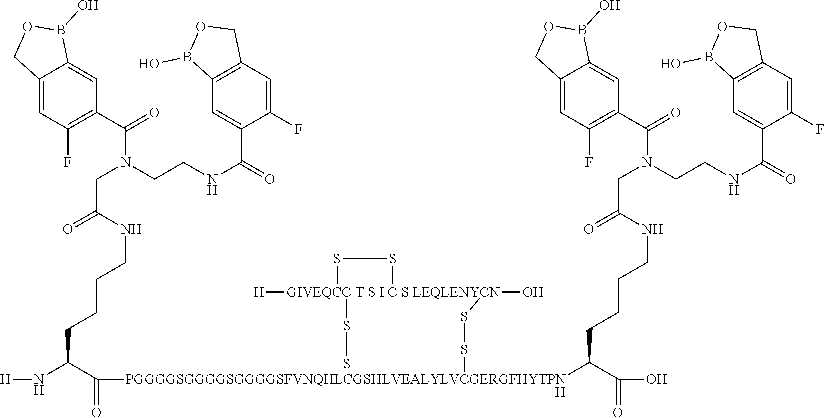

Example 203 was made similarly to example 101 from B1-KPGGGGSGGGGSGGGGS A14E B25H desB30 human insulin and building block of example 23.

### Example 204

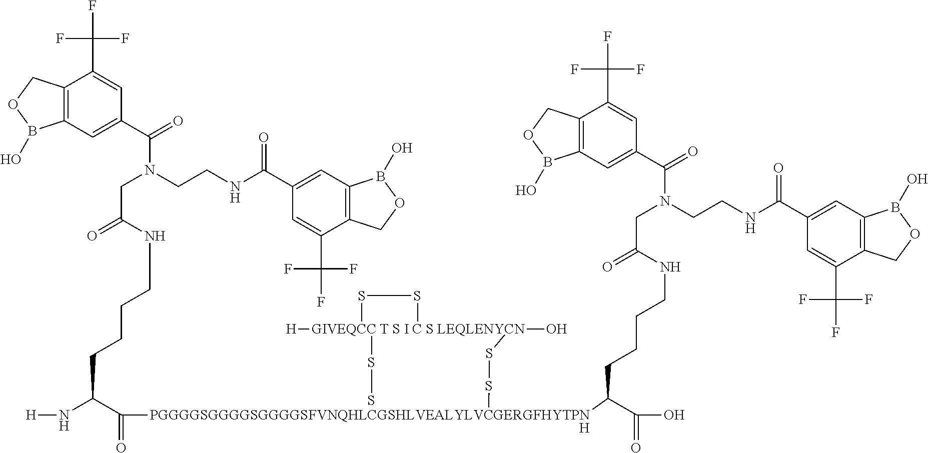

Example 204 was made similarly to example 101 from B1-KPGGGGSGGGGSGGGGS A14E B25H desB30 human insulin and building block of example 28.

### Example 205

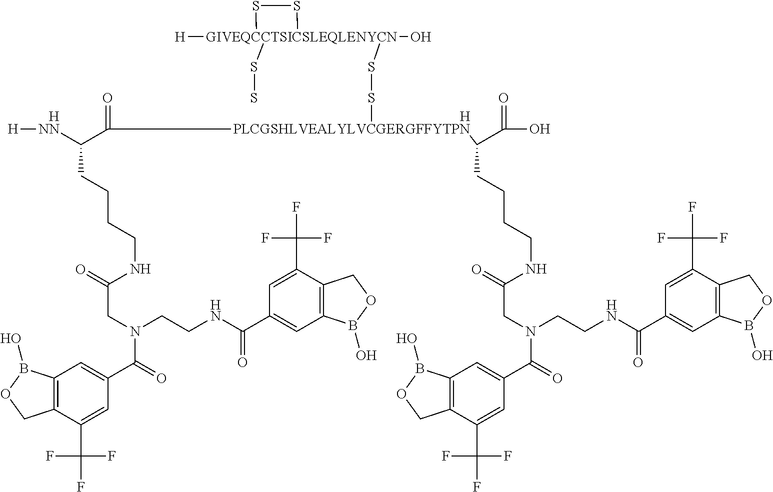

Example 205 was made similarly to example 101 from A14E desB1-B2 B4K B5P desB30 human insulin and building block of example 28.

### Example 206

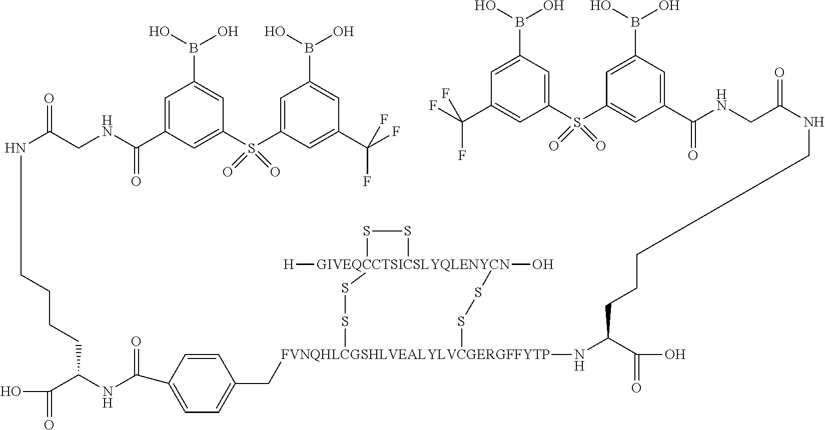

Example 206 was made similarly to example 101 from desB30 human insulin and building block of example 22.

### Example 207

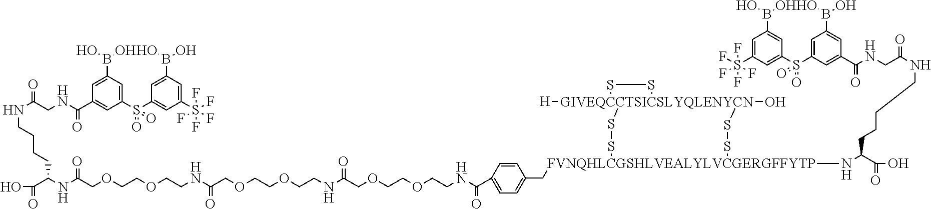

Example 207 was made similarly to example 101 from desB30 human insulin and building block of example 27.

### Example 208

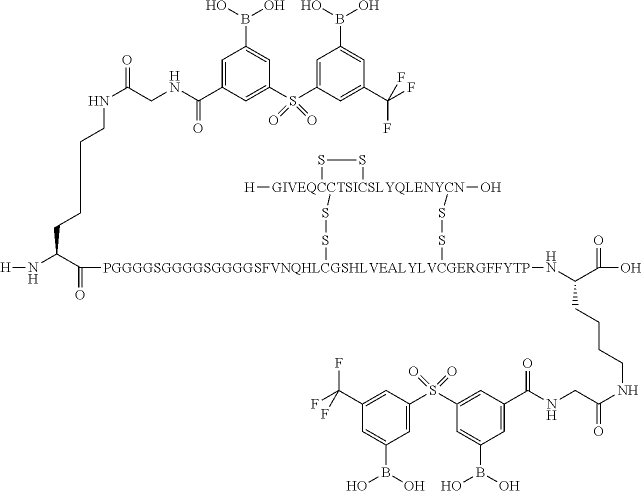

Example 208 was made similarly to example 101 from B1-KPGGGGSGGGGSGGGGS desB30 human insulin and building block of example 22.

### Example 209

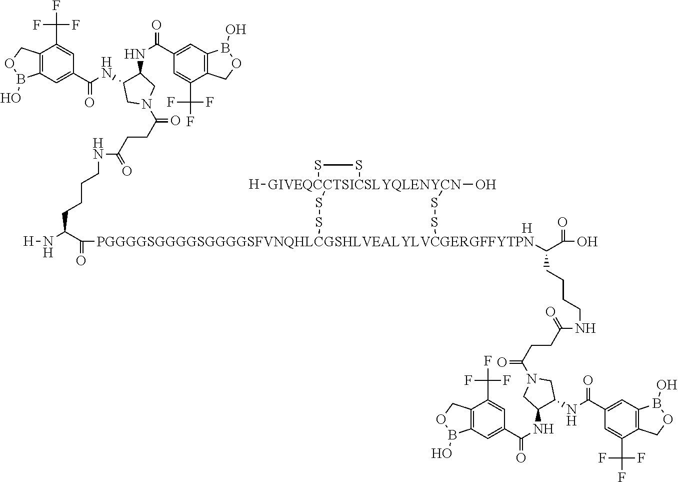

Example 209 was made similarly to example 101 from B1-KPGGGGSGGGGSGGGGS desB30 human insulin and building block of example 24.

### Example 210

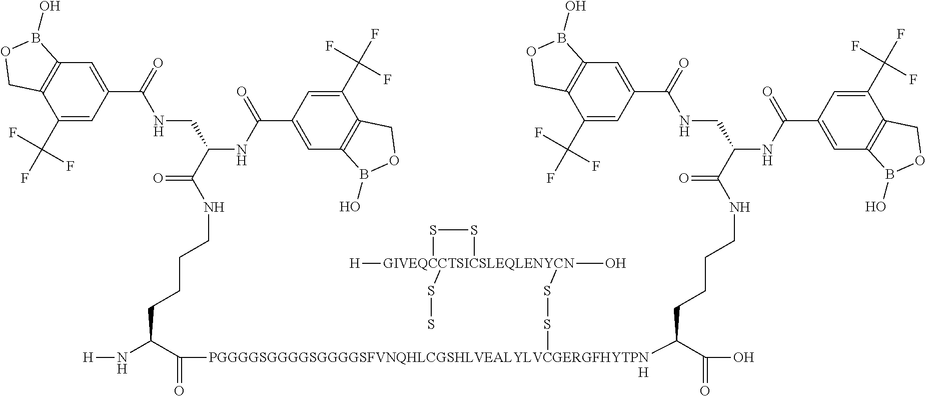

Example 210 was made similarly to example 101 from B1-KPGGGGSGGGGSGGGGS A14E B25H desB30 human insulin and building block of example 26.

### Example 211

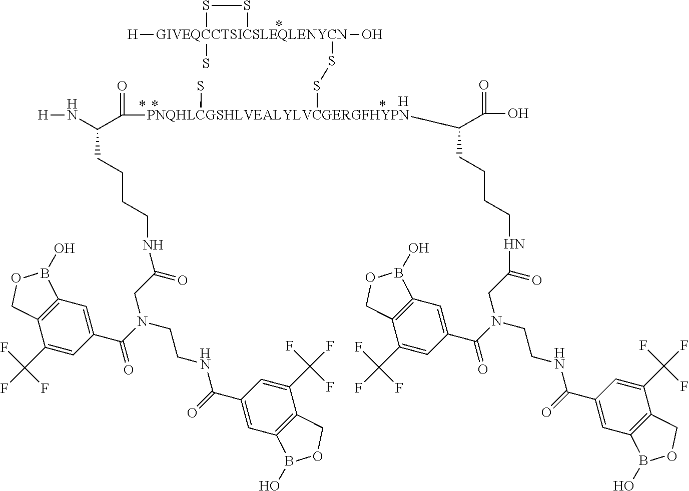

Example 211 was made similarly to example 101 from A14E B1K B2P B25H desB27 desB30 and building block of example 28.

### Example 212

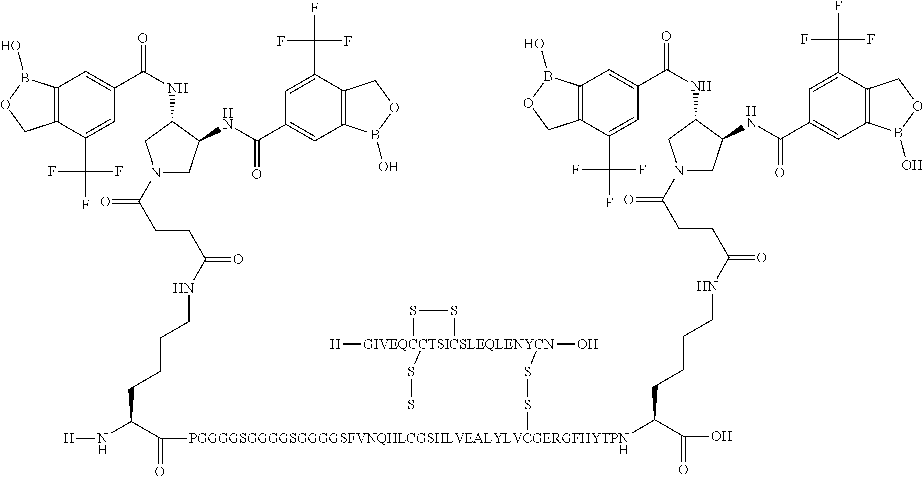

Example 212 was made similarly to example 101 from B1-KPGGGGSGGGGSGGGGS A14E B25H desB30 human insulin and building block of example 24.

### Example 213

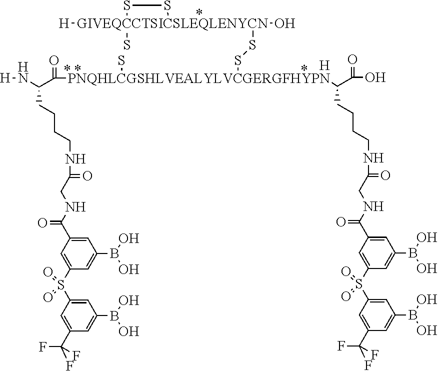

Example 213 was made similarly to example 101 from A14E B1K B2P B25H desB27 desB30 and building block of example 22.

### Example 214

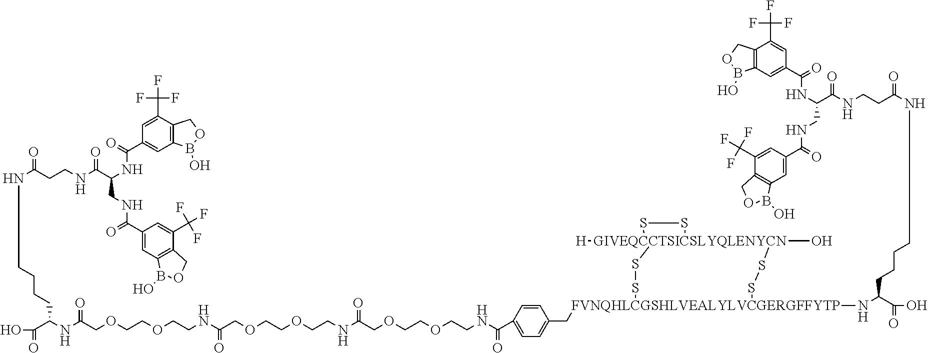

Example 214 was made similarly to example 105 from desB30 human insulin and building block of example 29.

### Example 215

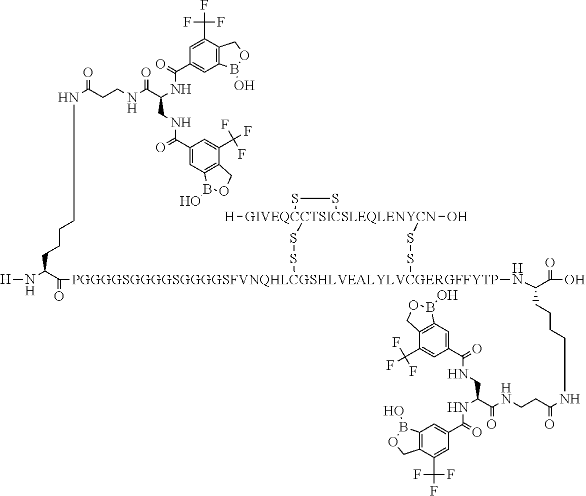

Example 215 was made similarly to example 101 from B1-KPGGGGSGGGGSGGGGS desB30 human insulin and building block of example 29.

### Example 216

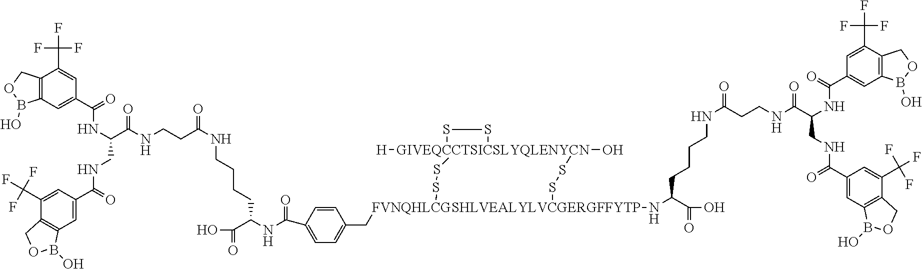

Example 216 was made similarly to example 105 from desB30 human insulin and building block of example 29.

### Example 217

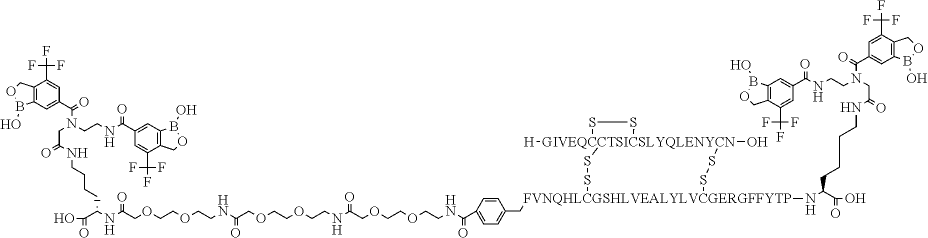

Example 217 was made similarly to example 105 from desB30 human insulin and building block of example 28.

### Example 218

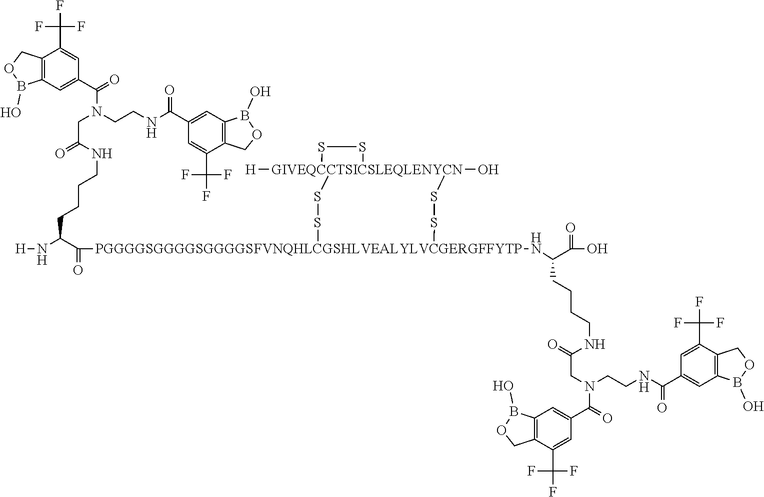

Example 218 was made similarly to example 101 from B1-KPGGGGSGGGGSGGGGS desB30 human insulin and building block of example 28.

### Example 219

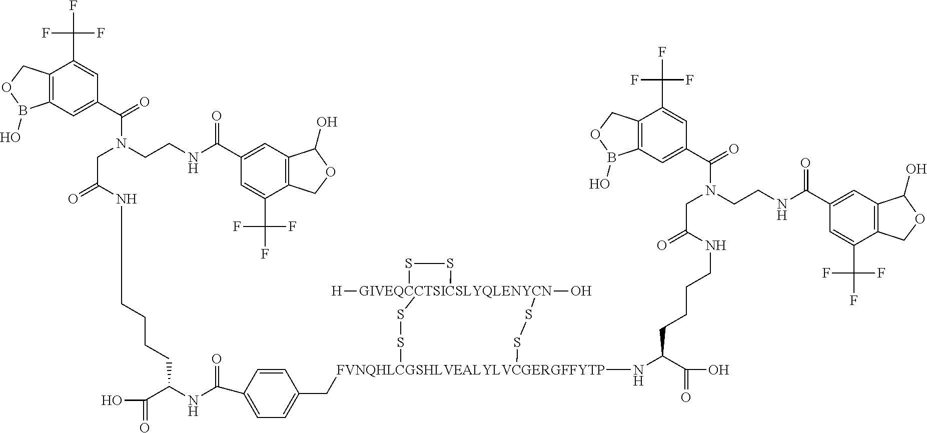

Example 219 was made similarly to example 105 from desB30 human insulin and building block of example 28.

### Example 220

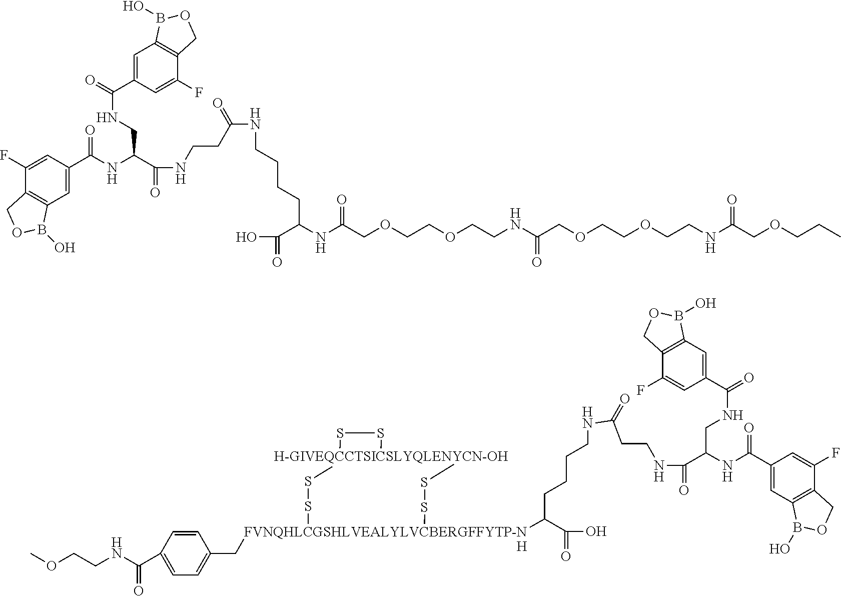

Example 220 was made similarly to example 105 from desB30 human insulin and building block of example 30.

### Example 221

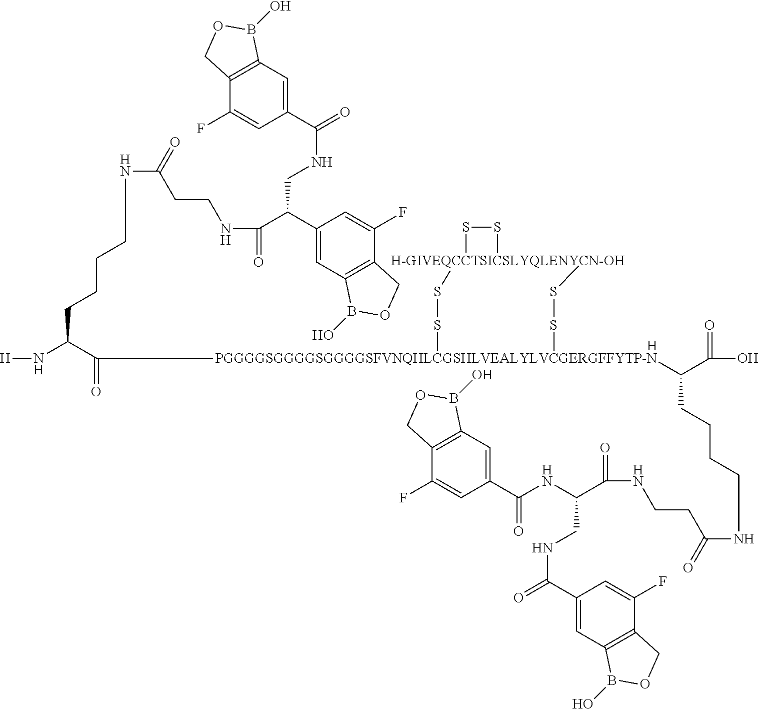

Example 221 was made similarly to example 101 from B1-KPGGGGSGGGGSGGGGS desB30 human insulin and building block of example 30.

### Example 222

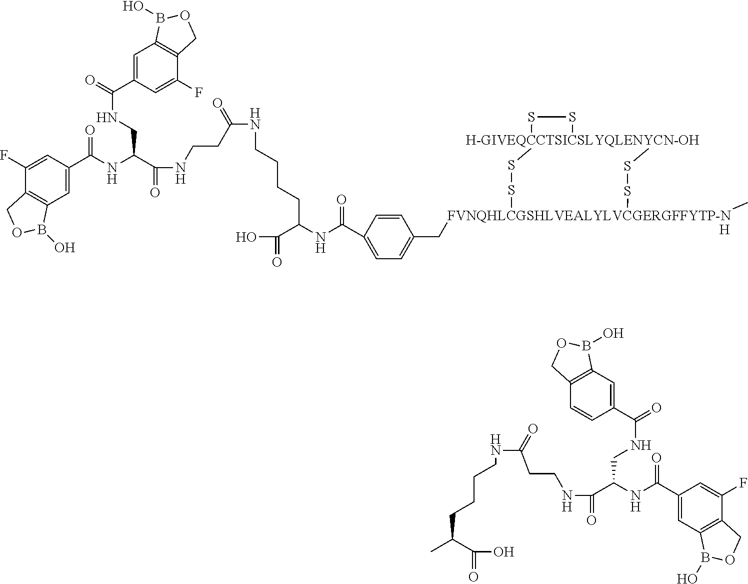

Example 222 was made similarly to example 105 from desB30 human insulin and building block of example 30.

### Example 223

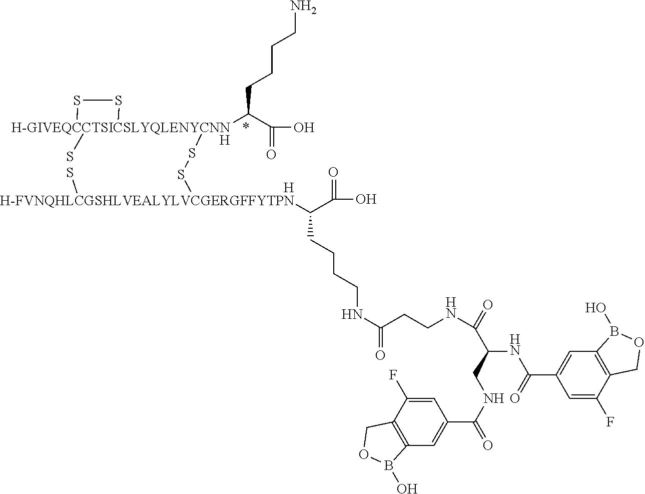

Example 223 was made similarly to example 101 from A22K desB30 human insulin and building block of example 30.

### Example 224

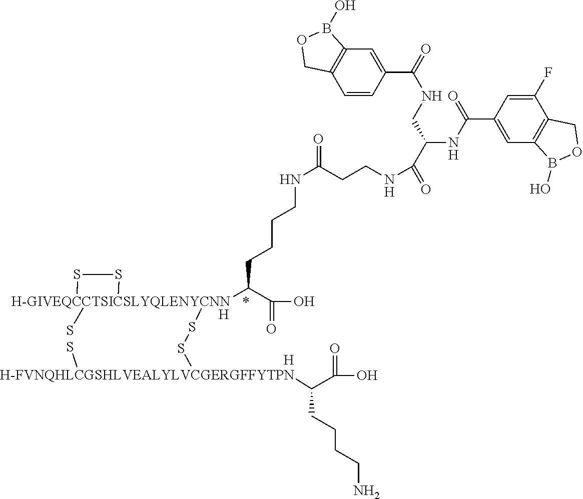

Example 224 was made similarly to example 101 from A22K desB30 human insulin and building block of example 30.

### Example 225

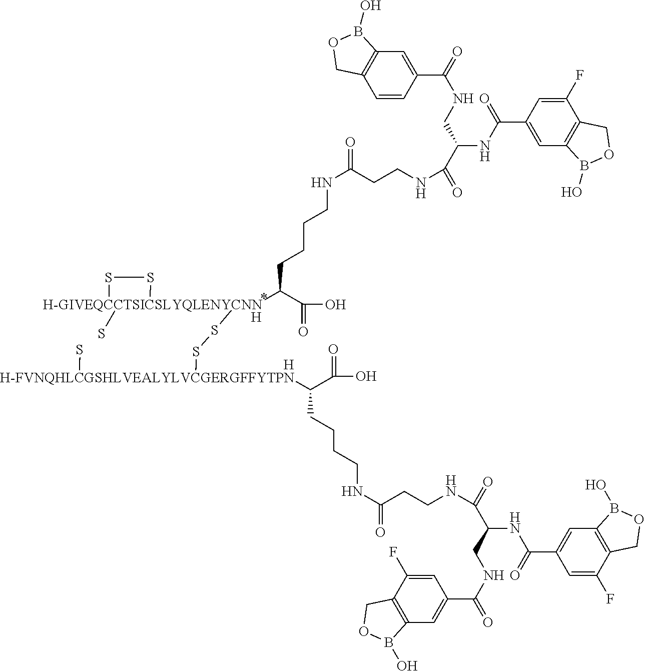

Example 225 was made similarly to example 101 from A22K desB30 human insulin and building block of example 30.

### Example 226

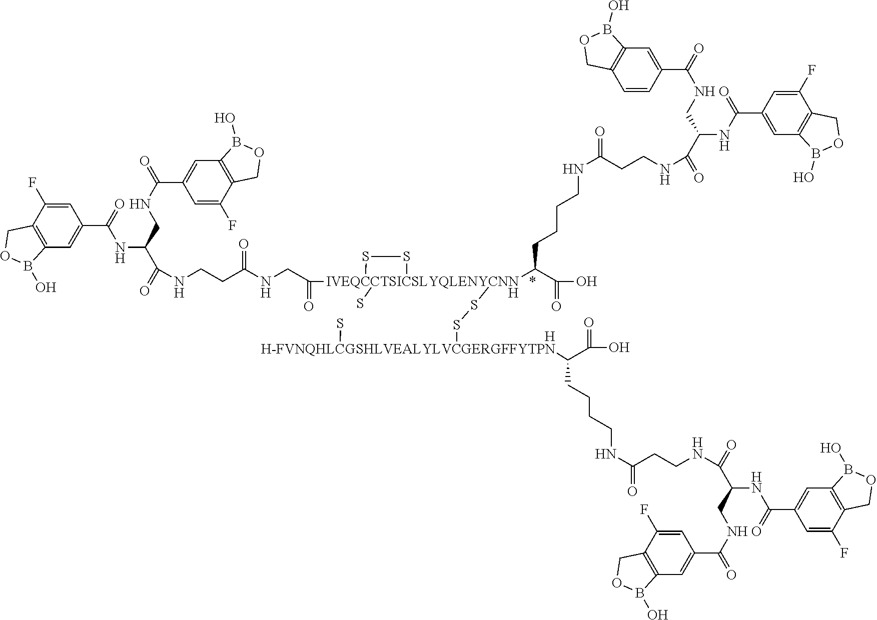

Example 226 was made similarly to example 101 from A22K desB30 human insulin and building block of example 30.

### Example 227

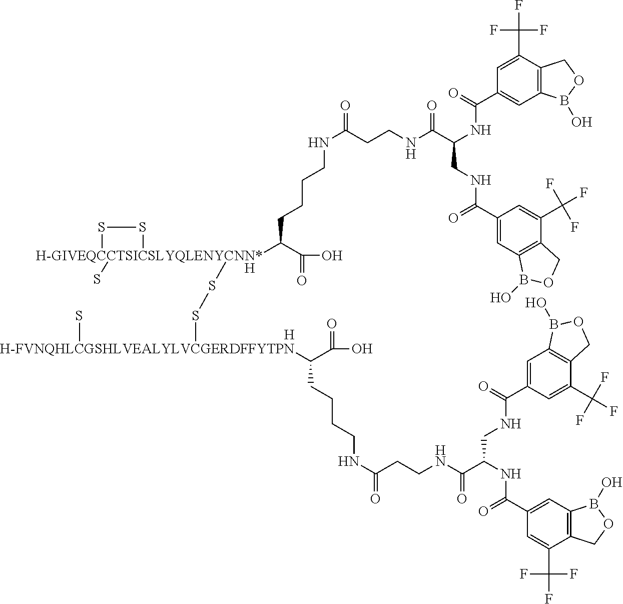

Example 227 was made similarly to example 101 from A22K desB30 human insulin and building block of example 29.

### Example 228

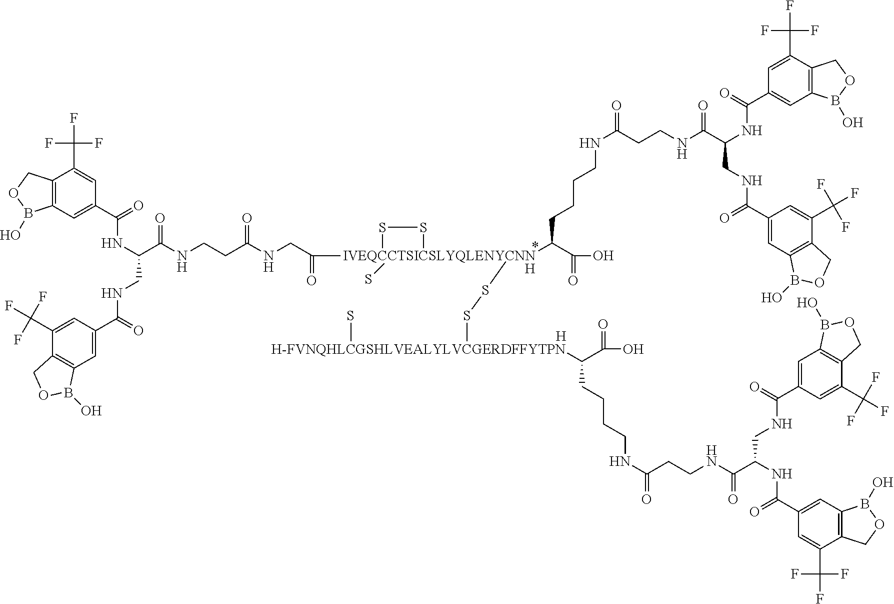

Example 228 was made similarly to example 101 from A22K desB30 human insulin and building block of example 29.

### Example 229

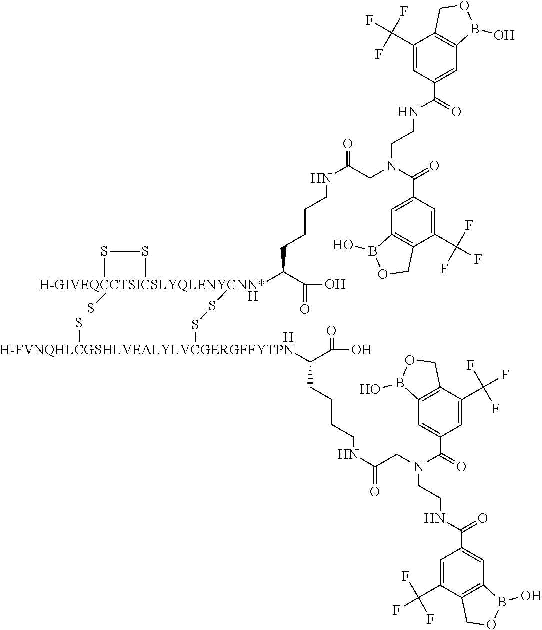

Example 229 was made similarly to example 101 from A22K desB30 human insulin and building block of example 28.

### Example 230

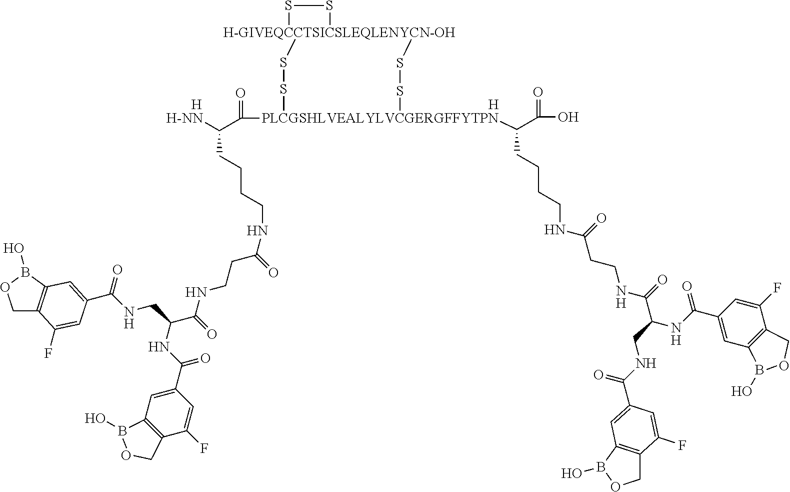

Example 230 was made similarly to example 101 from A14E desB1-B2 B4K B5P desB30 human insulin and building block of example 30.

### Example 231

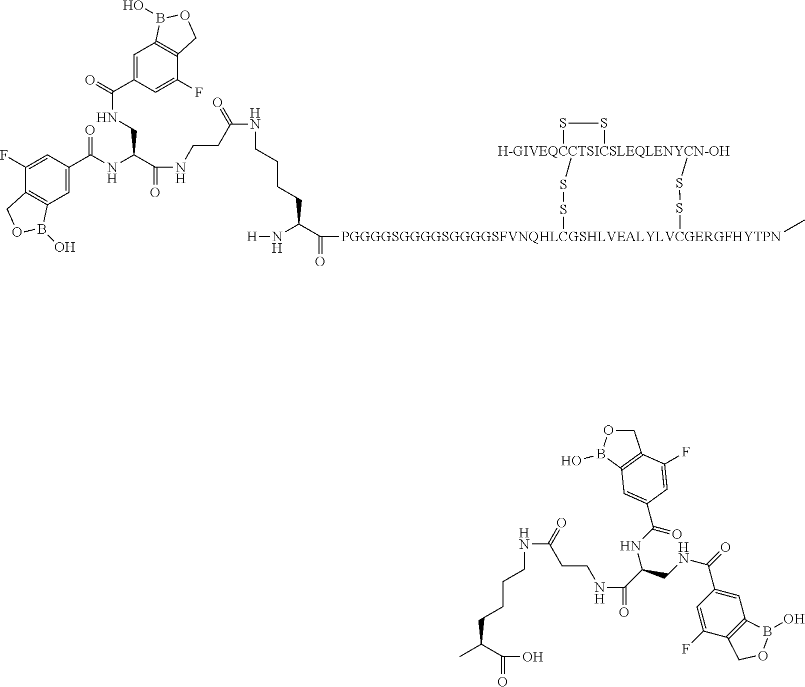

Example 231 was made similarly to example 101 from B1-KPGGGGSGGGGSGGGGS A14E B25H desB30 human insulin and building block of example 30.

### Example 232

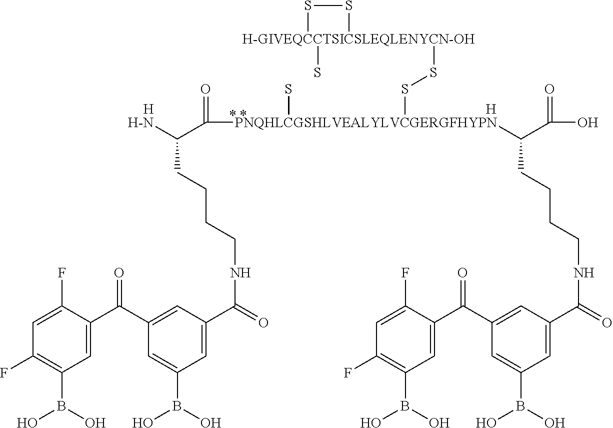

Example 232 was made similarly to example 101 from A14E B1K B2P B25H desB27 desB30 human insulin and building block of example 20.

### Example 233

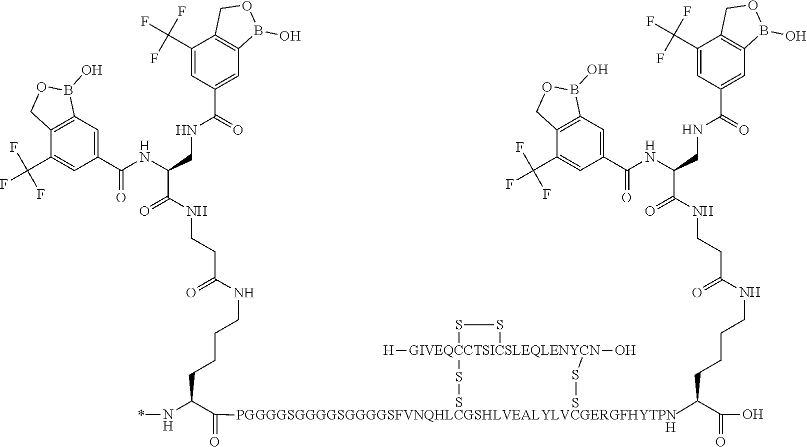

Example 233 was made similarly to example 101 from B1-KPGGGGSGGGGSGGGGS A14E B25H desB30 human insulin and building block of example 29.

### Example 234

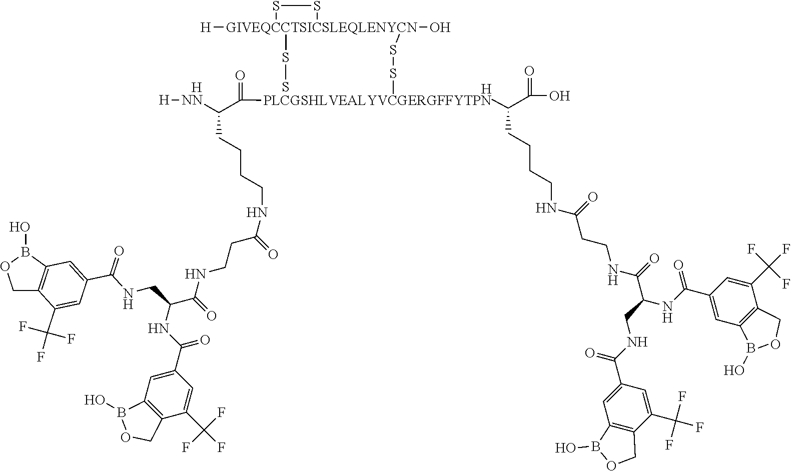

Example 234 was made similarly to example 101 from A14E desB1-B2 B4K B5P desB30 human insulin and building block of example 29.

### Example 235

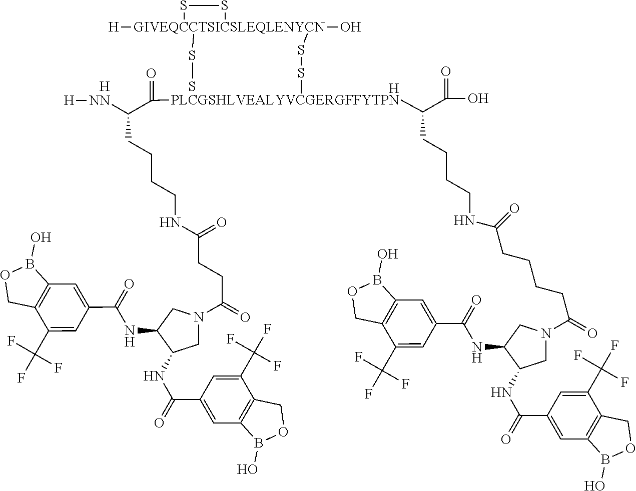

Example 235 was made similarly to example 101 from A14E desB1-B2 B4K B5P desB30 human insulin and building block of example 24.

### Example 236

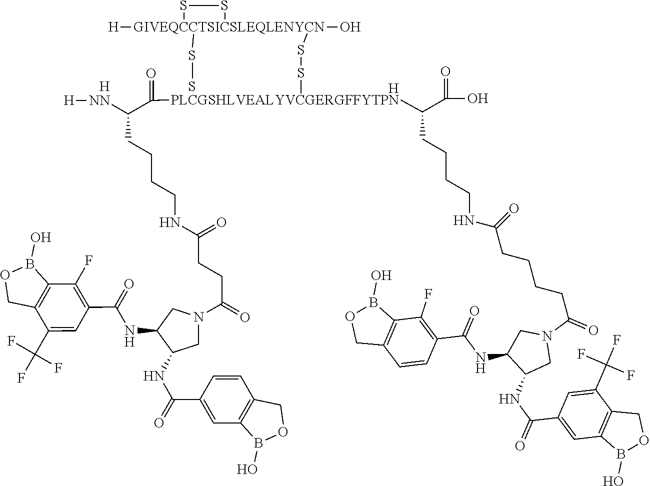

Example 236 was made similarly to example 101 from A14E desB1-B2 B4K B5P desB30 human insulin and building block of example 31.

### Example 237

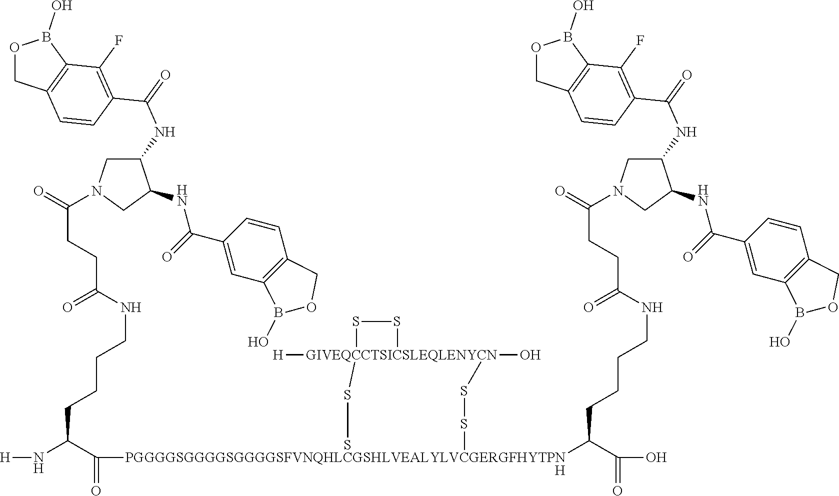

Example 237 was made similarly to example 101 from B1-KPGGGGSGGGGSGGGGS A14E B25H desB30 human insulin and building block of example 31.

### Example 238

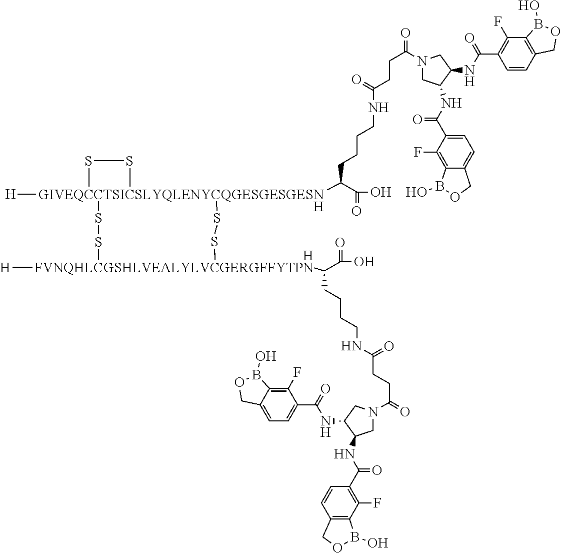

Example 238 was made similarly to example 101 from A21Q (GES)3K desB30 human insulin and building block of example 31.

### Example 239

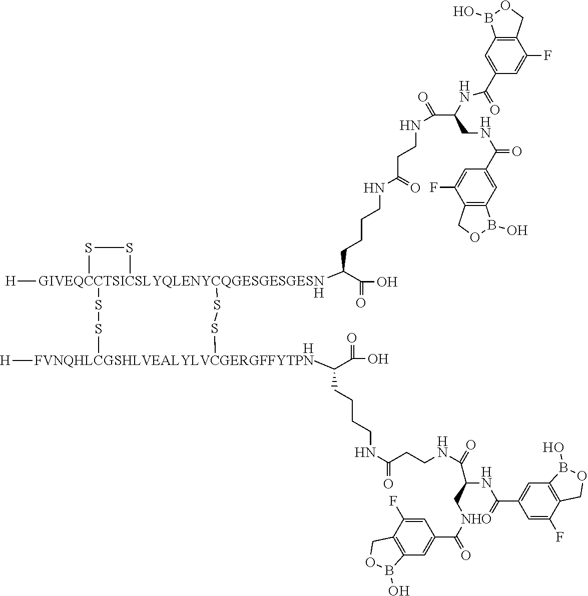

Example 239 was made similarly to example 101 from A21Q (GES)3K desB30 human insulin and building block of example 30.

### Example 240

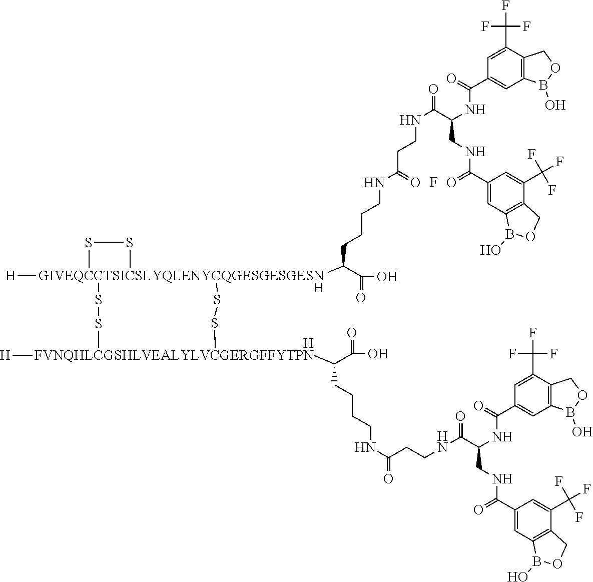

Example 240 was made similarly to example 101 from A21Q (GES)3K desB30 human insulin and building block of example 29.

### Example 241

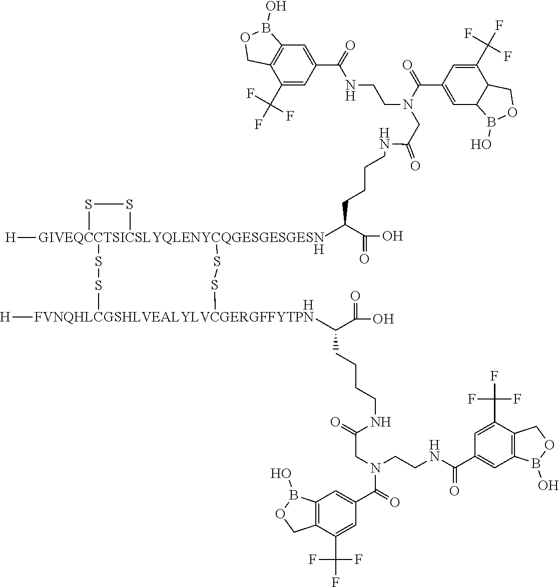

Example 241 was made similarly to example 101 from A21Q (GES)3K desB30 human insulin and building block of example 28.

### Example 242

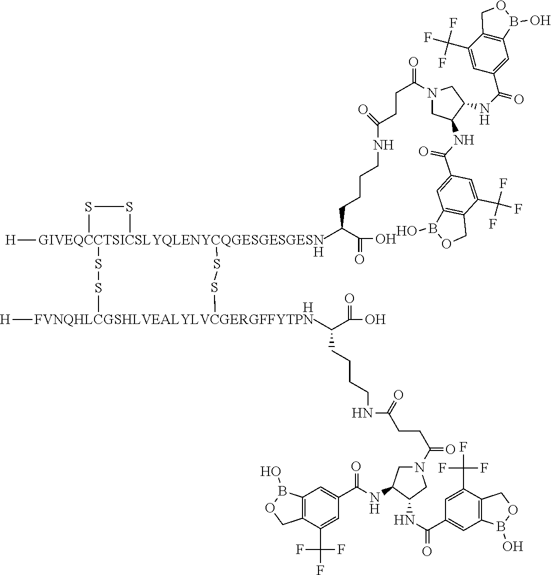

Example 242 was made similarly to example 101 from A21Q (GES)3K desB30 human insulin and building block of example 24.

### Example 243

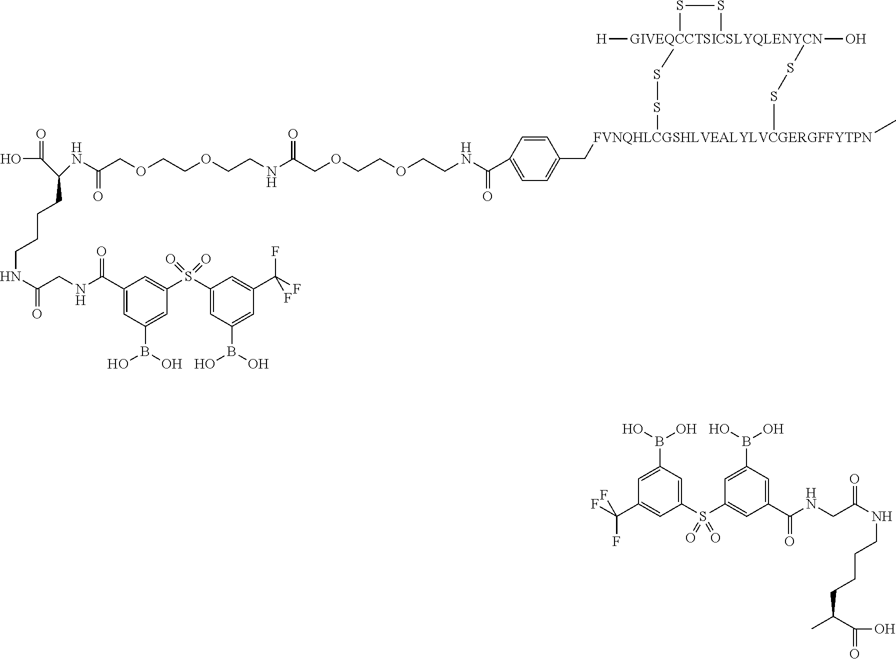

Example 243 was made similarly to example 105 from desB30 human insulin and building block of example 22.

### Example 244

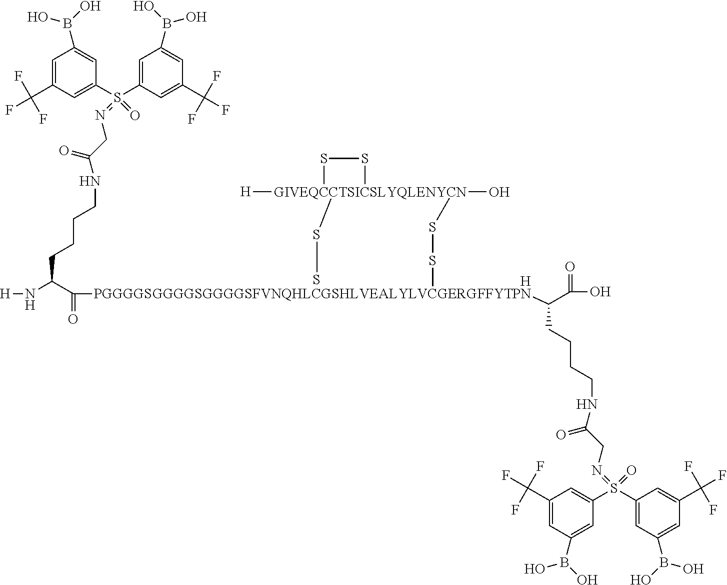

Example 244 was made similarly to example 101 from B1-KPGGGGSGGGGSGGGGS desB30 human insulin and building block of example 33.

### Example 245

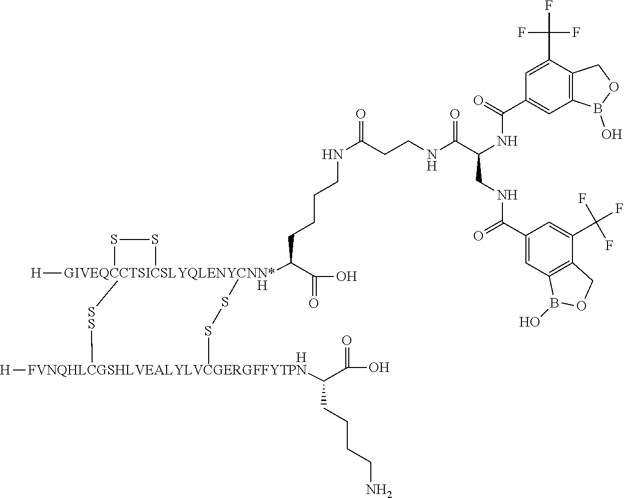

Example 245 was made similarly to example 101 from A22K desB30 human insulin and building block of example 29.

### Example 246

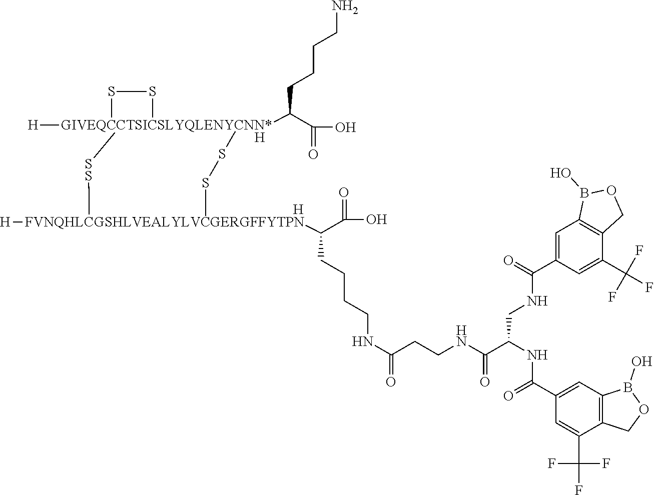

Example 246 was made similarly to example 101 from A22K desB30 human insulin and building block of example 29.

### Example 247

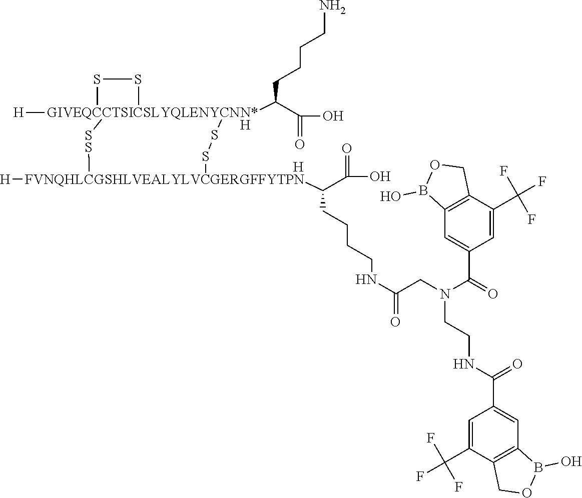

Example 247 was made similarly to example 101 from A22K desB30 human insulin and building block of example 28.

### Example 248

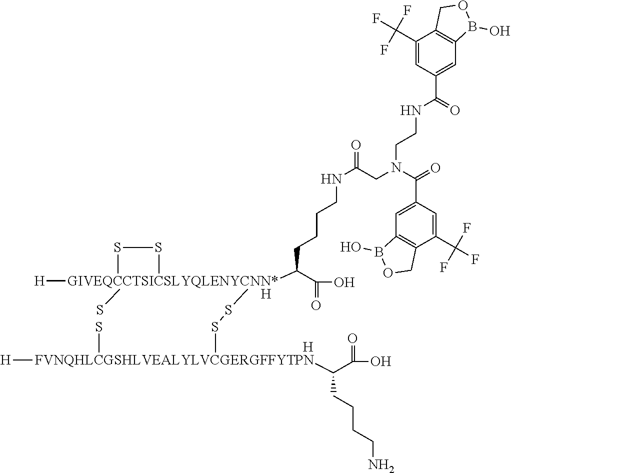

Example 248 was made similarly to example 101 from A22K desB30 human insulin and building block of example 28.

### Example 249

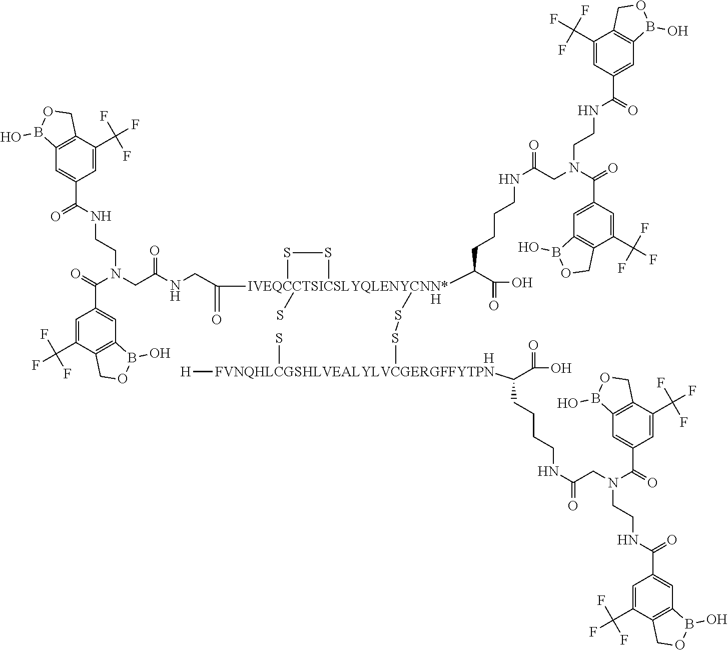

Example 249 was made similarly to example 101 from A22K desB30 human insulin and building block of example 28.

### Example 250

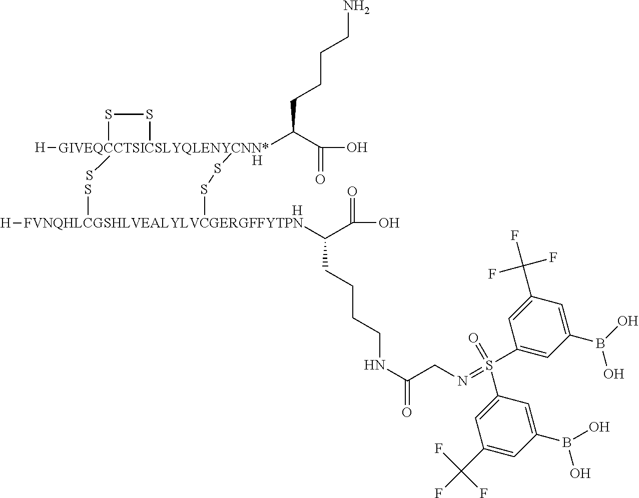

Example 250 was made similarly to example 101 from A22K desB30 human insulin and building block of example 33.

### Example 251

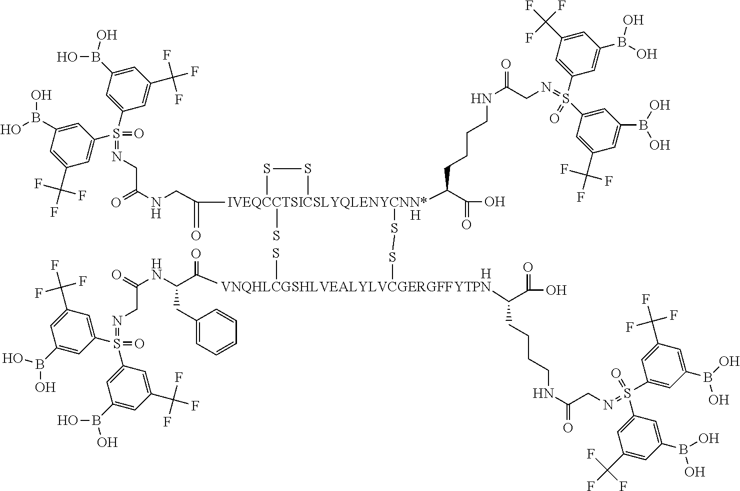

Example 251 was made similarly to example 101 from A22K desB30 human insulin and building block of example 33.

### Example 252

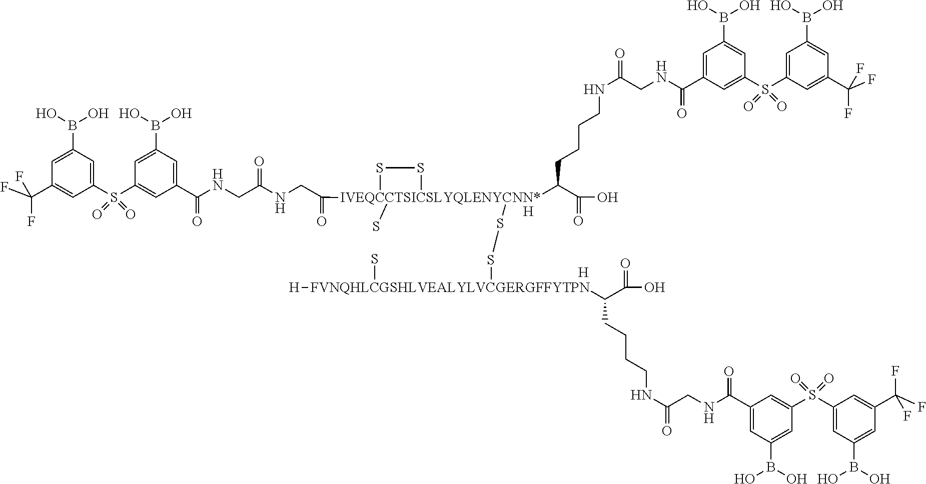

Example 252 was made similarly to example 101 from A22K desB30 human insulin and building block of example 22.

### Example 253

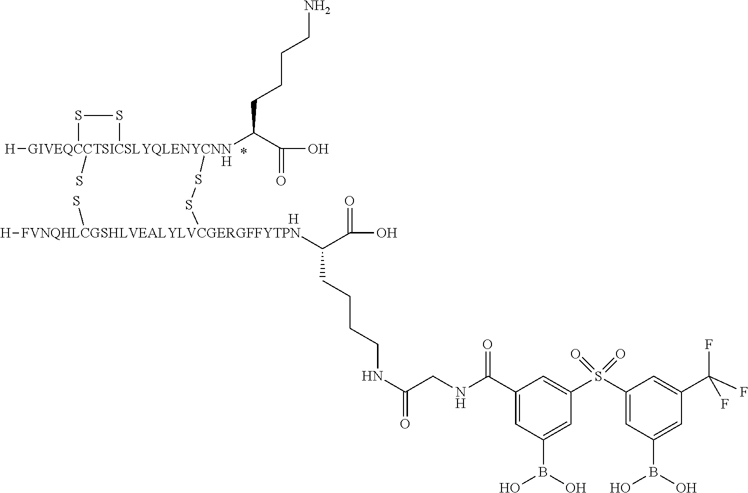

Example 253 was made similarly to example 101 from A22K desB30 human insulin and building block of example 22.

### Example 254

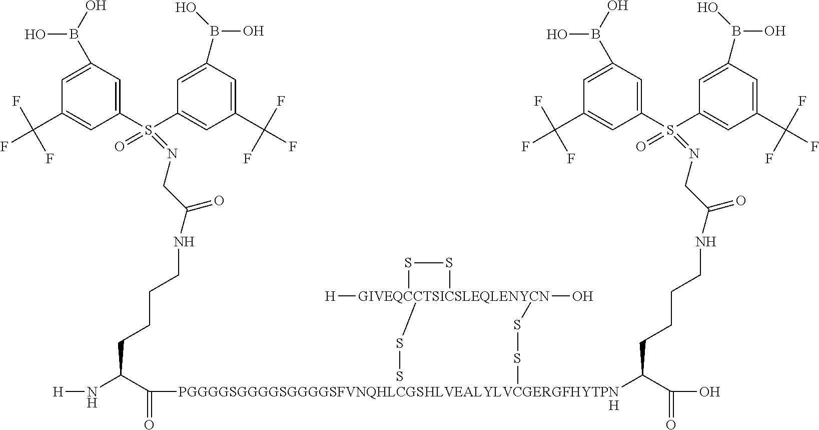

Example 254 was made similarly to example 101 from B1-KPGGGGSGGGGSGGGGS A14E B25H desB30 human insulin and building block of example 33.

### Example 255

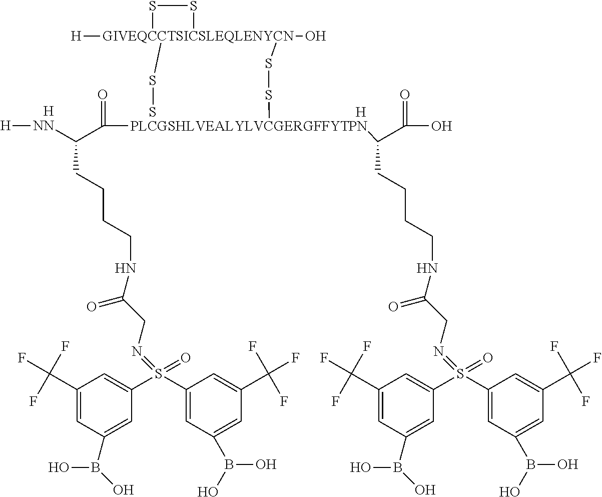

Example 255 was made similarly to example 101 from A14E desB1-B2 B4K B5P desB30 human insulin and building block of example 33.

### Example 256

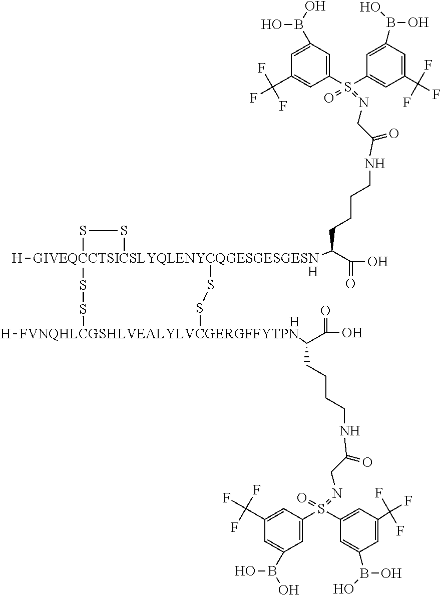

Example 256 was made similarly to example 101 from A21Q (GES)3K desB30 human insulin and building block of example 33.

### Example 257

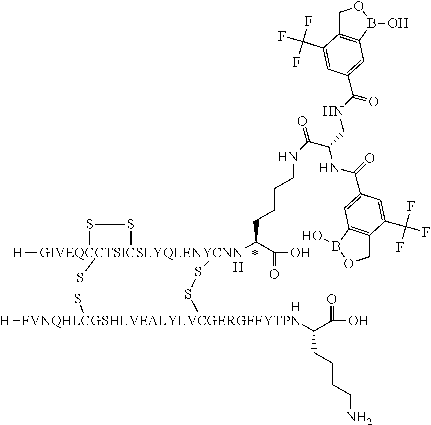

Example 257 was made similarly to example 101 from A22K desB30 human insulin and building block of example 26.

### Example 258

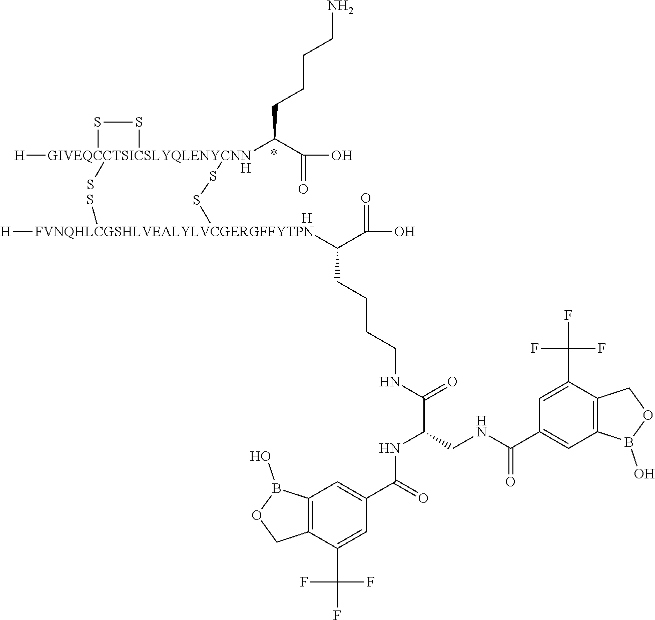

Example 258 was made similarly to example 101 from A22K desB30 human insulin and building block of example 26.

### Example 259

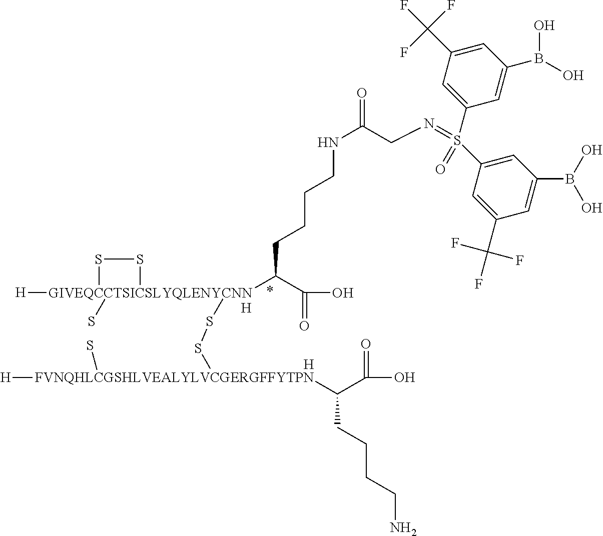

Example 259 was made similarly to example 101 from A22K desB30 human insulin and building block of example 33.

### Example 260

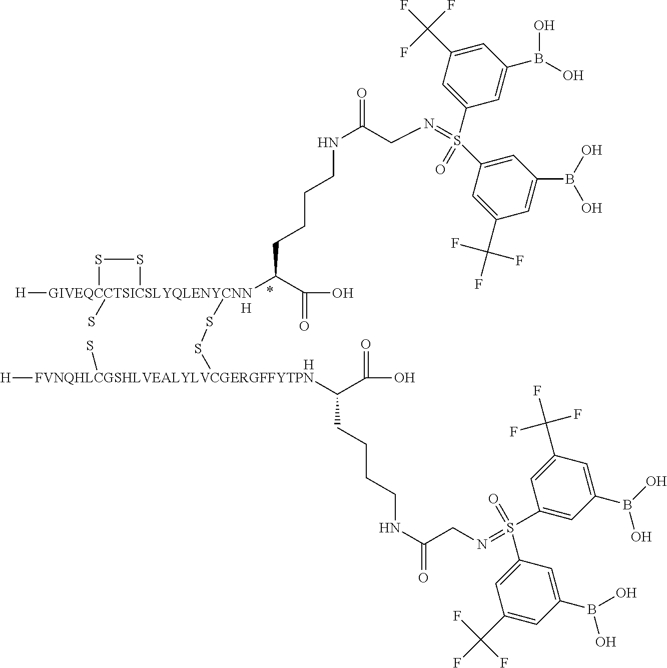

Example 260 was made similarly to example 101 from A22K desB30 human insulin and building block of example 33.

### Example 261

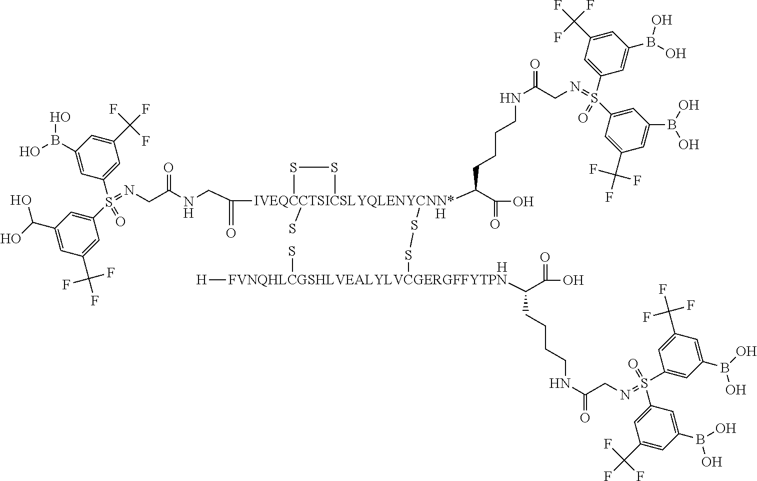

Example 261 was made similarly to example 101 from A22K desB30 human insulin and building block of example 22.

### Example 262

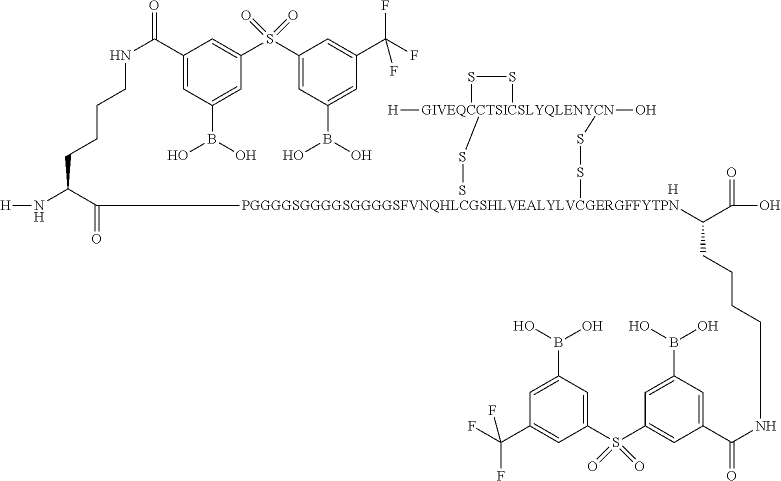

Example 262 was made similarly to example 101 from B1-KPGGGGSGGGGSGGGGS desB30 human insulin and building block of example 34.

### Example 263

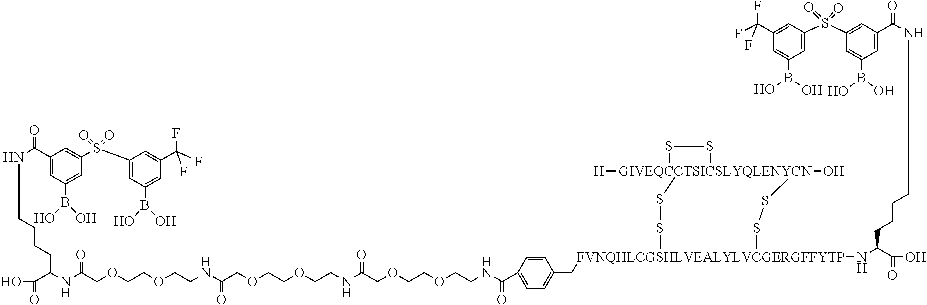

Example 263 was made similarly to example 105 from desB30 human insulin and building block of example 34.

### Example 264

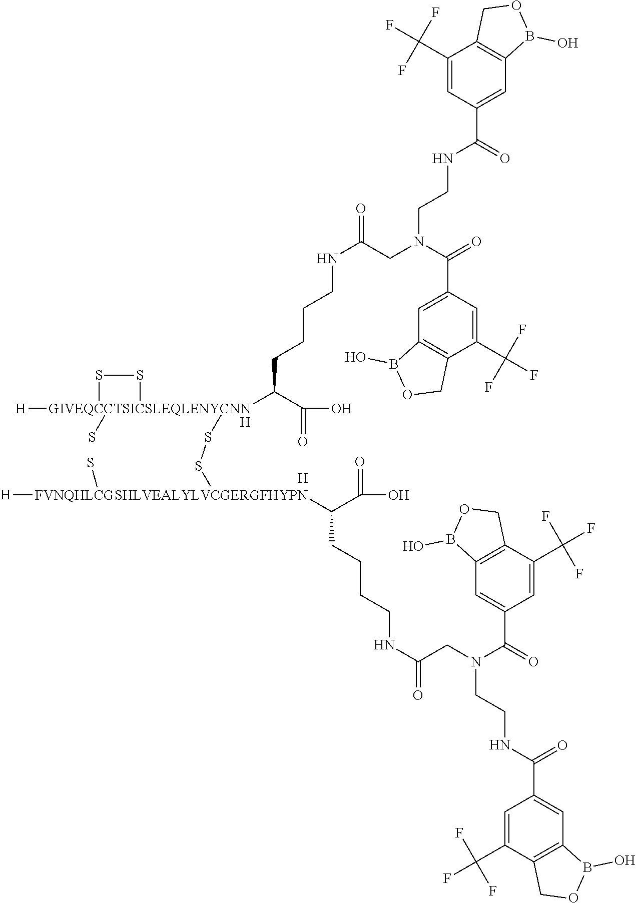

Example 264 was made similarly to example 101 from A14E A22K B25H desB27 desB30 human insulin and building block of example 28.

### Example 265

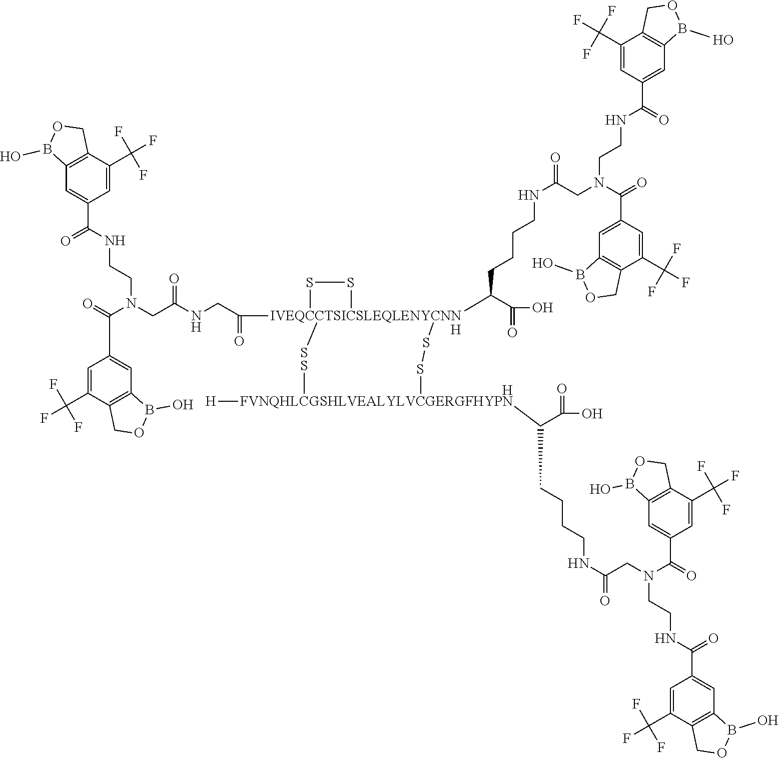

Example 265 was made similarly to example 101 from A14E A22K B25H desB27 desB30 human insulin and building block of example 28.

### Example 266

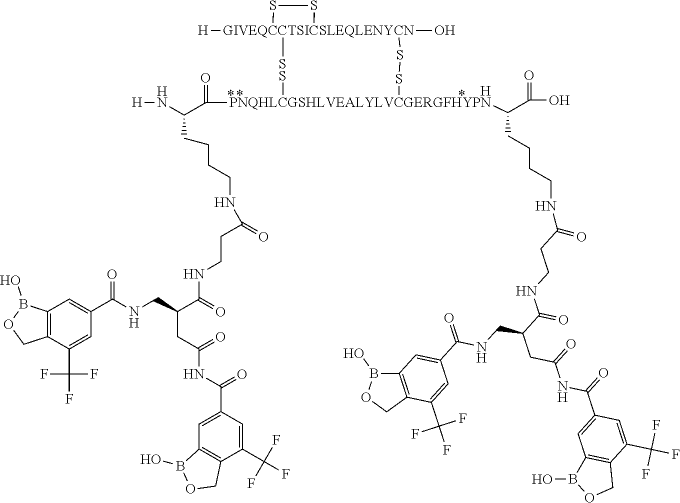

Example 266 was made similarly to example 101 from A14E B1K B2P B25H desB27 des B30 human insulin and building block of example 29.

### Example 267

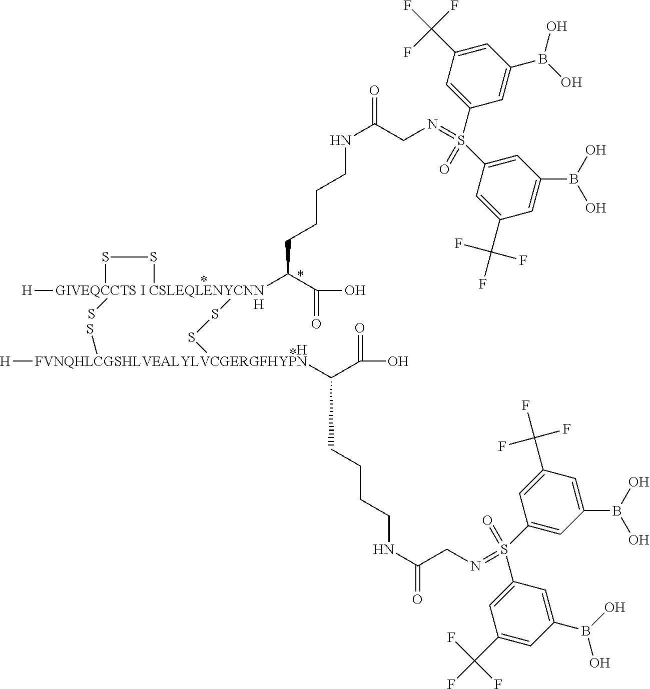

Example 267 was made similarly to example 101 from A14E A22K B25H desB27 desB30 human insulin and building block of example 33.

### Example 268

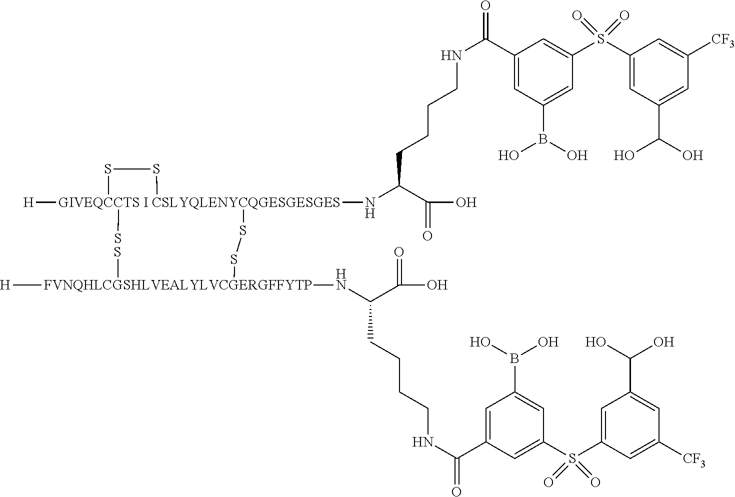

Example 268 was made similarly to example 101 from A21Q (GES)3K desB30 human insulin and building block of example 34.

### Example 269

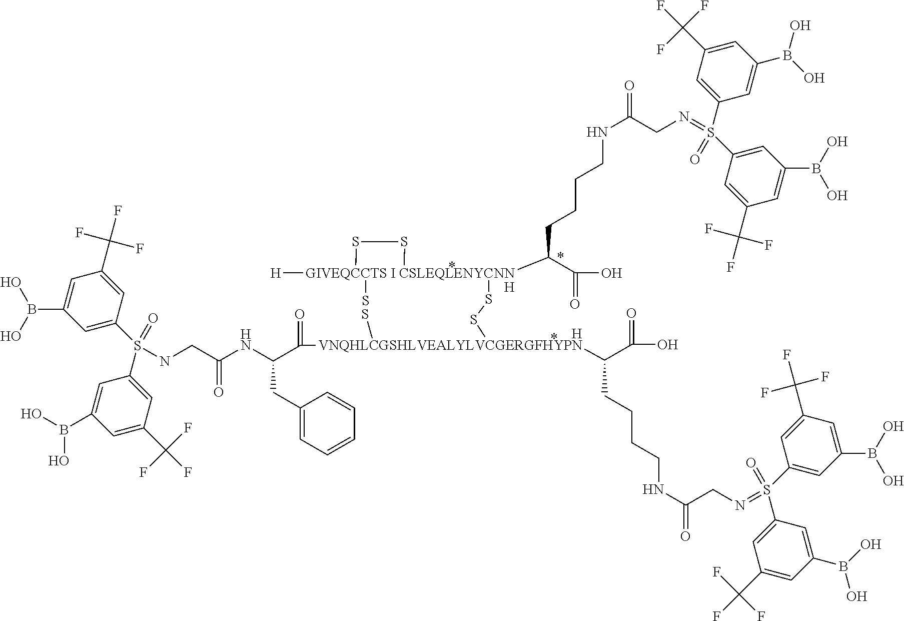

Example 269 was made similarly to example 101 from A14E A22K B25H desB27 des B30 human insulin and building block of example 33.

### Example 270

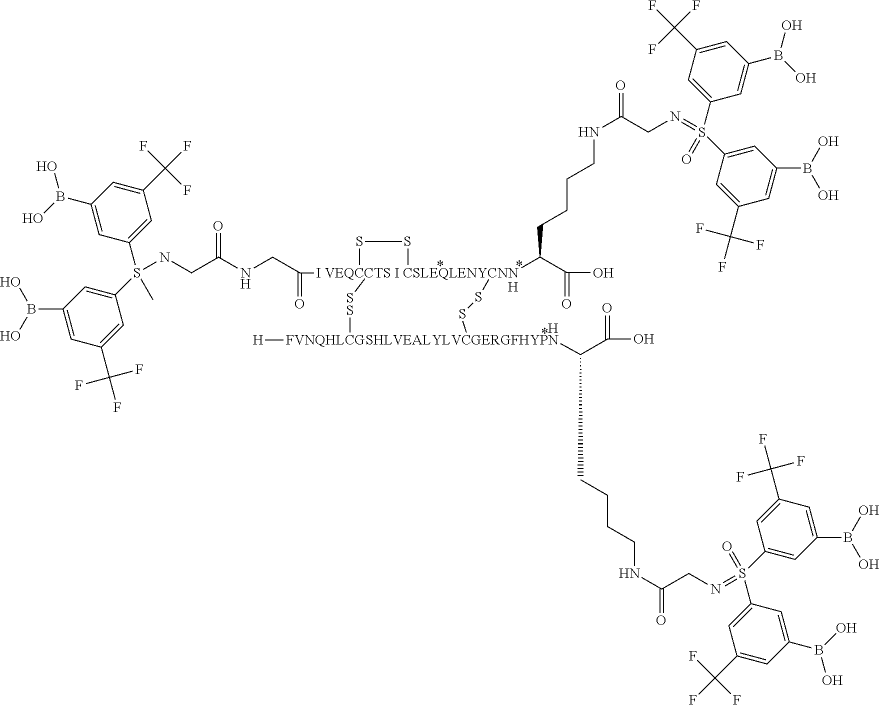

Example 270 was made similarly to example 101 from A14E A22K B25H desB27 desB30 human insulin and building block of example 33.

### Example 271

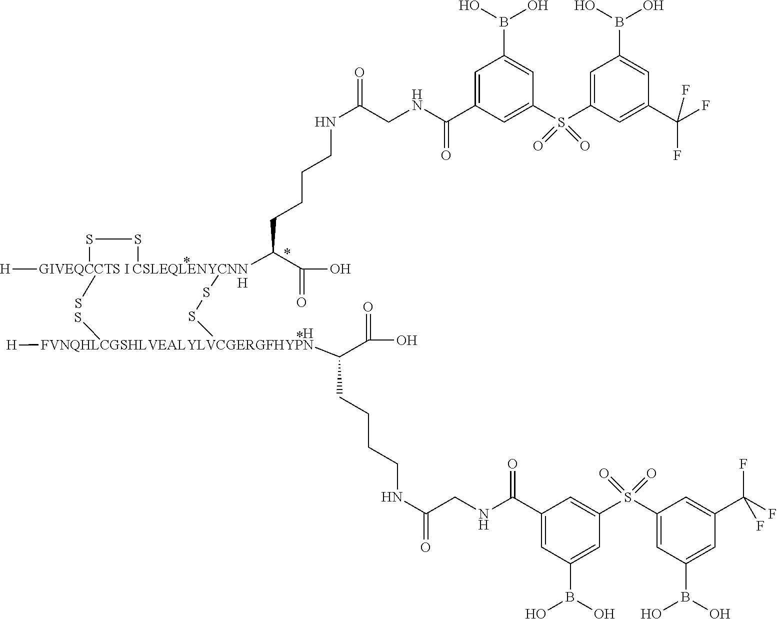

Example 271 was made similarly to example 101 from A14E A22K B25H desB27 desB30 human insulin and building block of example 22.

### Example 272

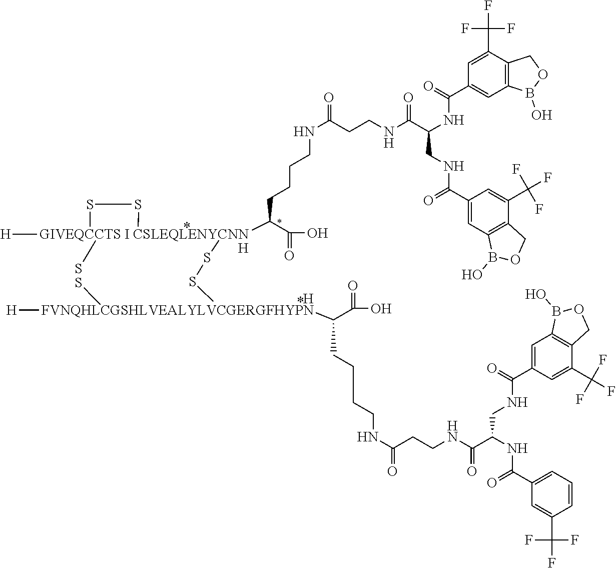

Example 272 was made similarly to example 101 from A14E A22K B25H desB27 desB30 human insulin and building block of example 29.

### Example 273

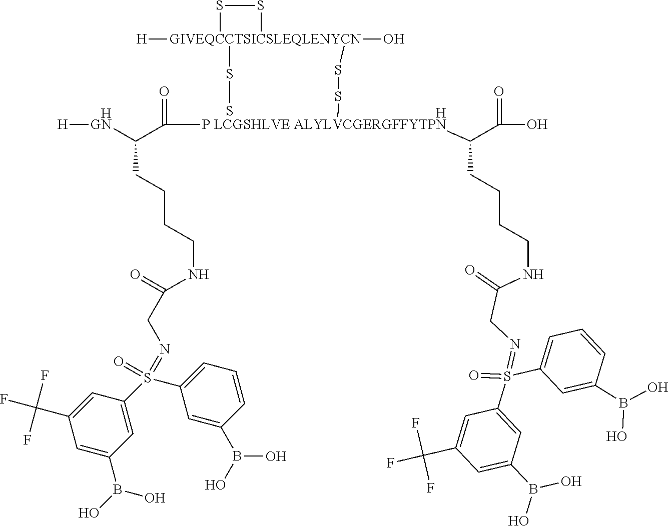

Example 273 was made similarly to example 101 from A14E desB1-B2 B3G B4K B5P desB30 human insulin and building block of example 33.

### Example 274

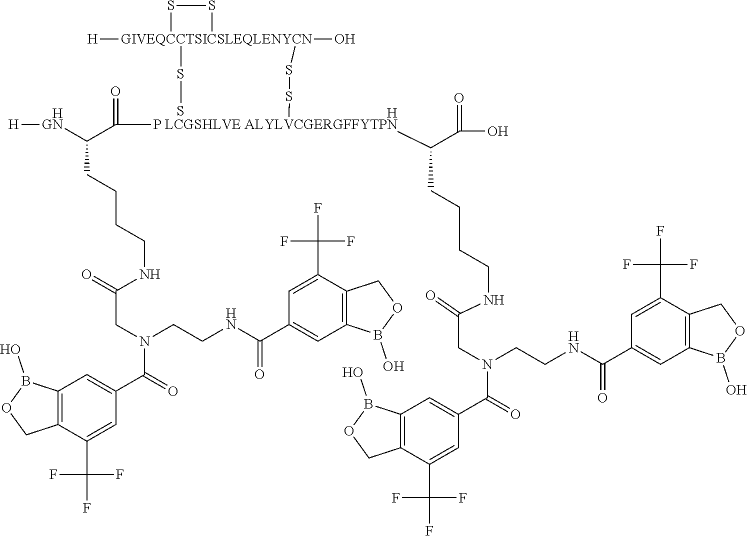

Example 274 was made similarly to example 101 from A14E desB1-B2 B3G B4K B5P desB30 human insulin and building block of example 28.

### Example 275

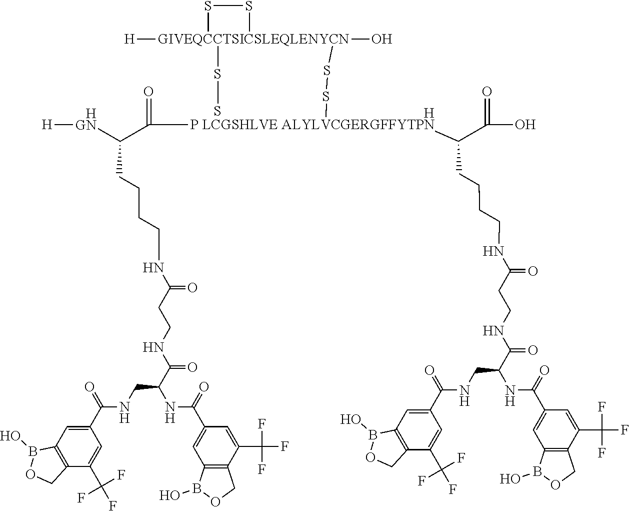

Example 275 was made similarly to example 101 from A14E desB1-B2 B3G B4K B5P desB30 human insulin and building block of example 29.

### Example 276

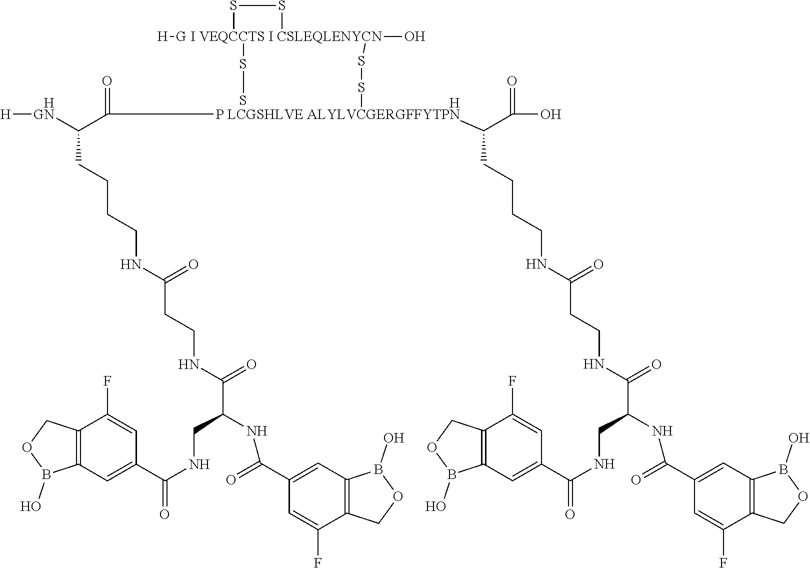

Example 276 was made similarly to example 101 from A14E desB1-B2 B3G B4K B5P desB30 human insulin and building block of example 30.

### Example 277

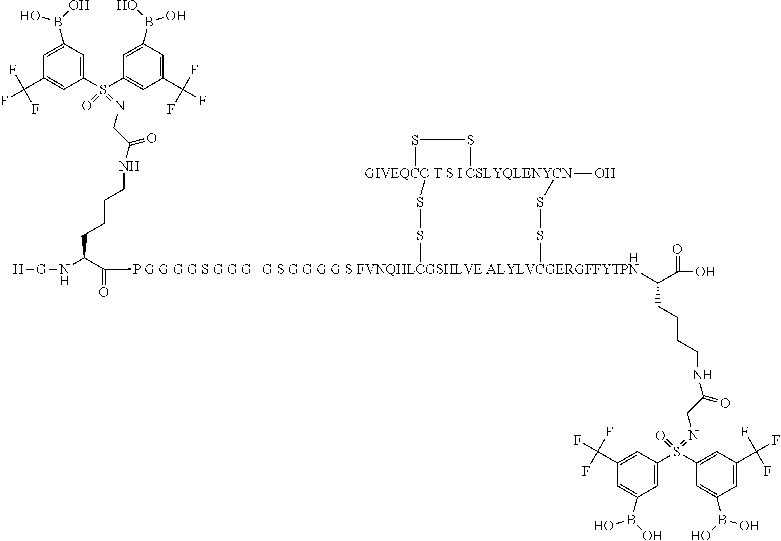

Example 277 was made similarly to example 101 from B1-GKPGGGGSGGGGSGGGGS desB30 human insulin and building block of example 33.

### Example 278

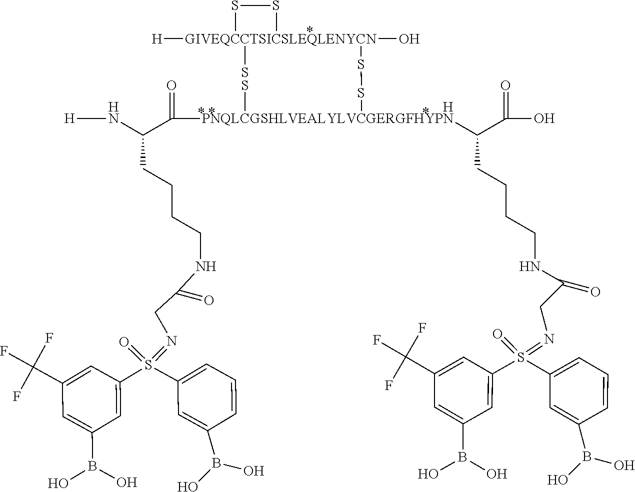

Example 278 was made similarly to example 101 from A14E B1K B2P B25H desB27 desB30 human insulin and building block of example 33.

### Example 279

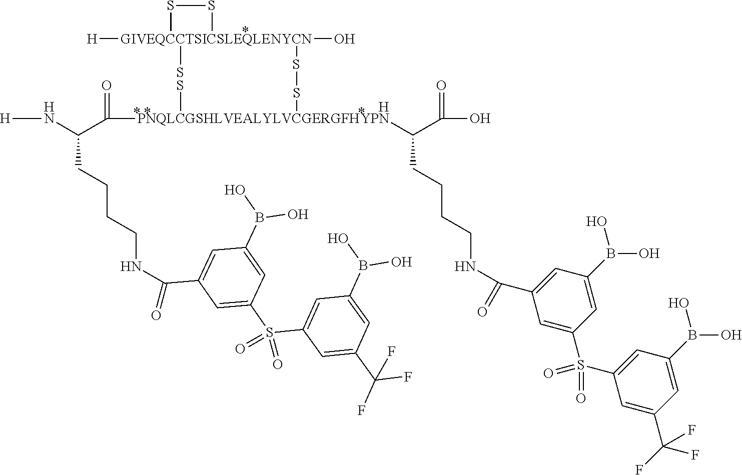

Example 279 was made similarly to example 101 from A14E B1K B2P B25H desB27 desB30 human insulin and building block of example 34.

### Example 280

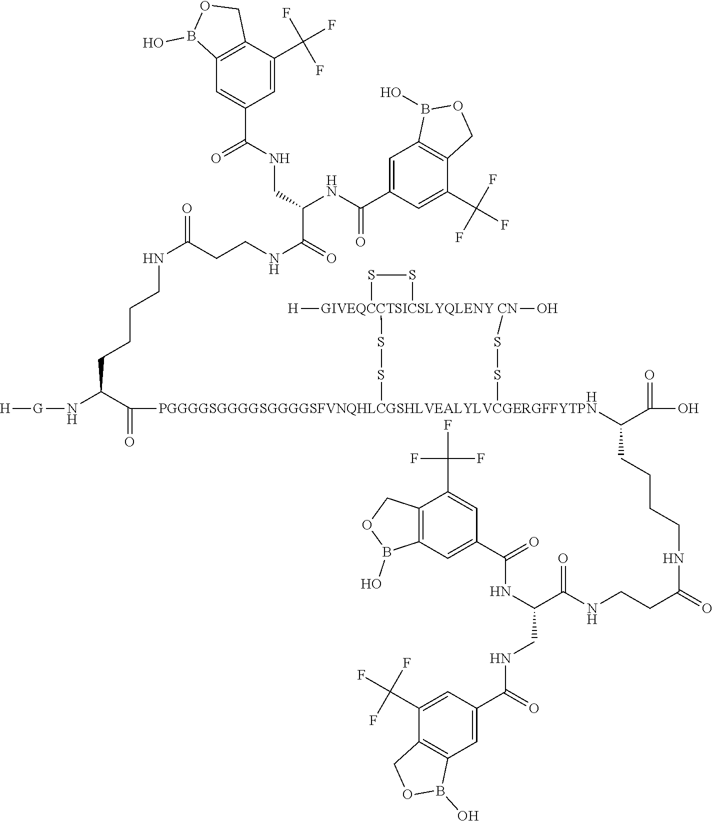

Example 280 was made similarly to example 101 from B1-GKPGGGGSGGGGSGGGGS desB30 human insulin and building block of example 29.

### Example 281

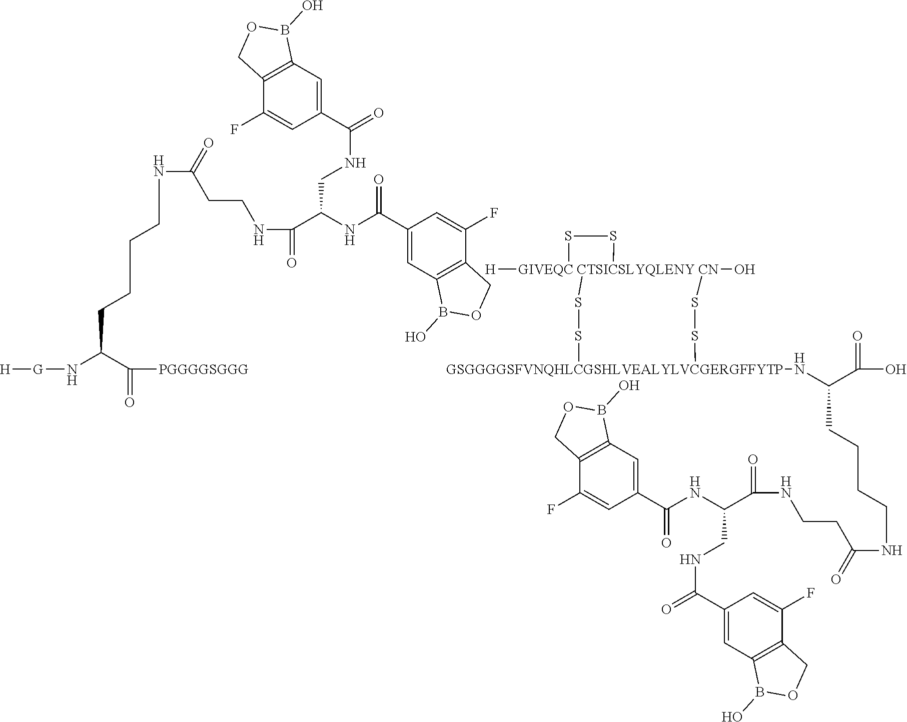

Example 281 was made similarly to example 101 from B1-GKPGGGGSGGGGSGGGGS desB30 human insulin and building block of example 30.

### Example 282

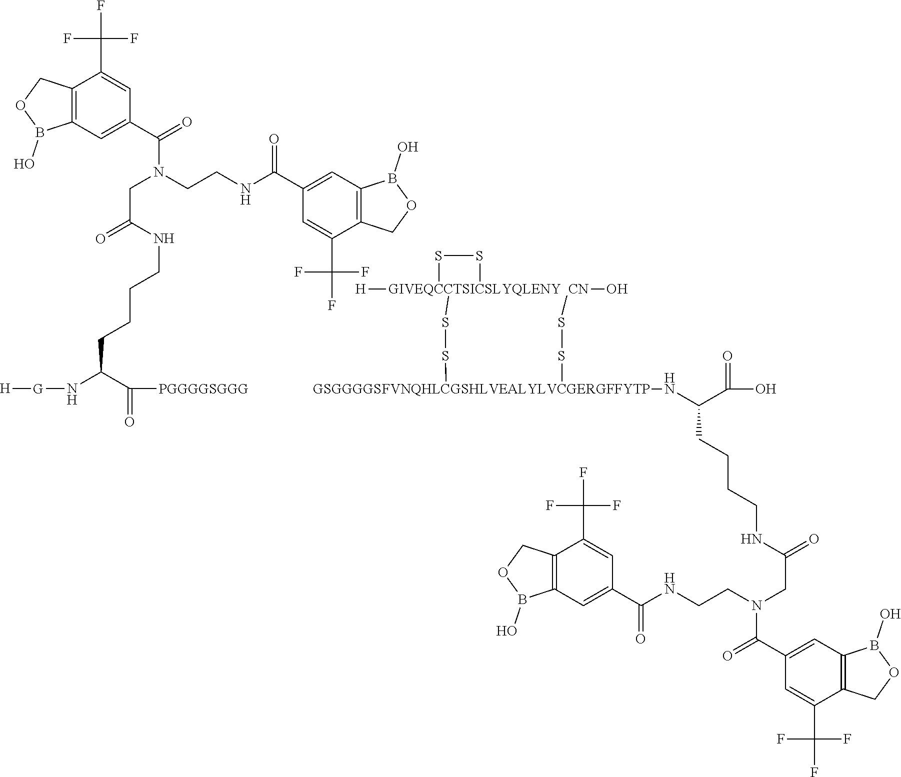

Example 282 was made similarly to example 101 from B1-GKPGGGGSGGGGSGGGGS desB30 human insulin and building block of example 28.

### Example 283

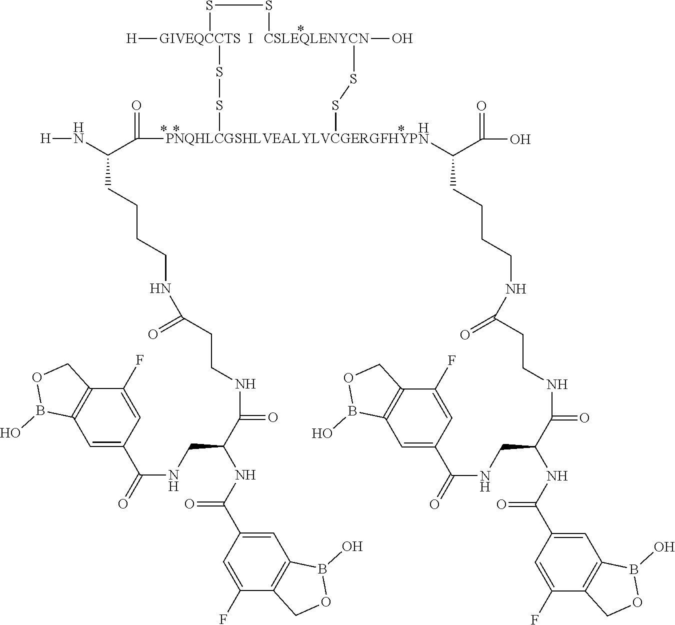

Example 283 was made similarly to example 101 from A14E B1K B2P B25H desB27 desB30 and building block of example 30.

### Example 284

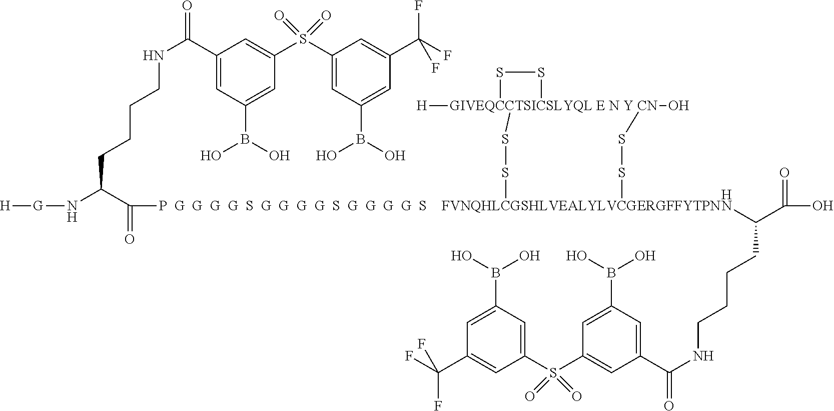

Example 284 was made similarly to example 101 from B1-CKPCCGCSGGGGSGGGGS desB30 human insulin and building block of example 34.

### Example 285

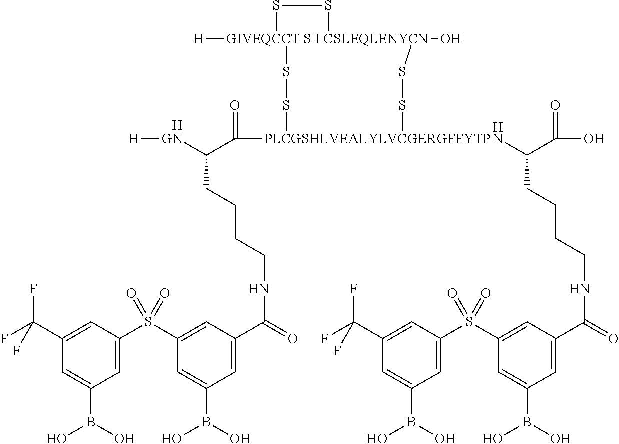

Example 285 was made similarly to example 101 from A14E desB1-B2 B3G B4K B5P desB30 and building block of example 34.

### Example 286

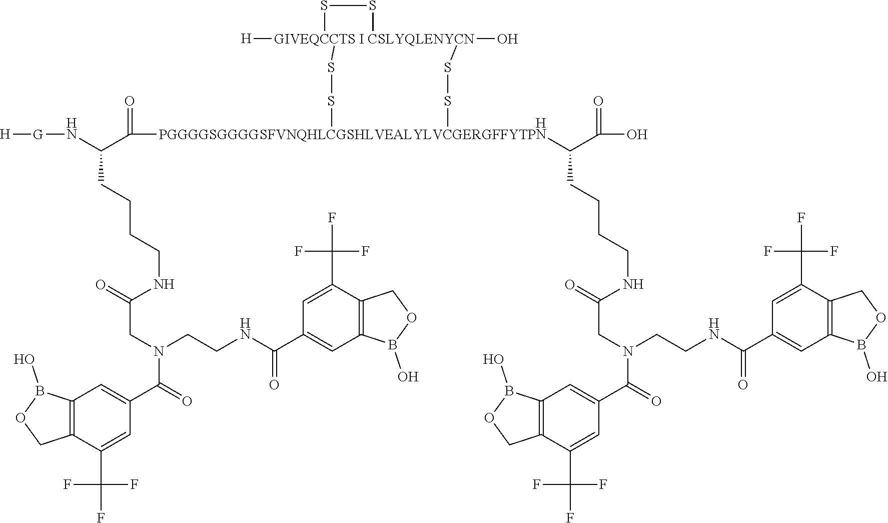

Example 286 was made similarly to example 101 from B1-GKPGGGGSGGGGS desB30 human insulin and building block of example 28.

### Example 287

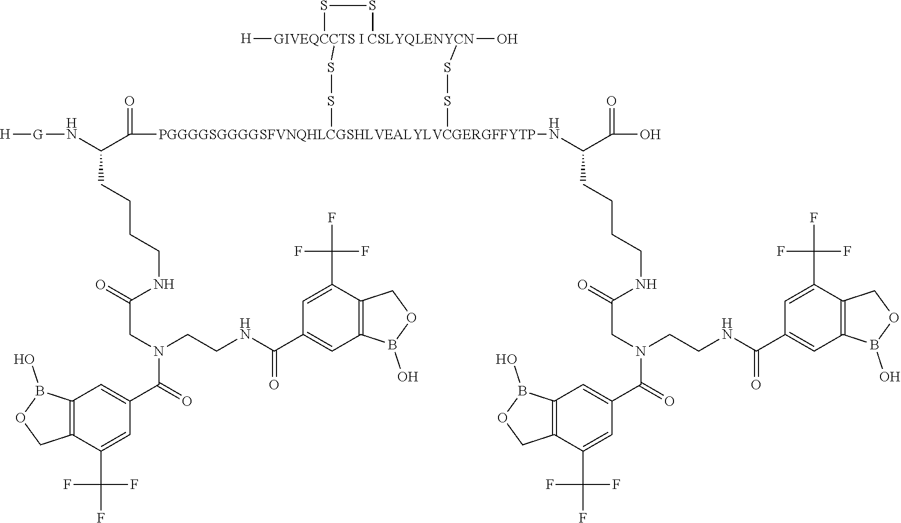

Example 287 was made similarly to example 101 from B1-GKPGGGGS desB30 human insulin and building block of example 28.

### Example 288

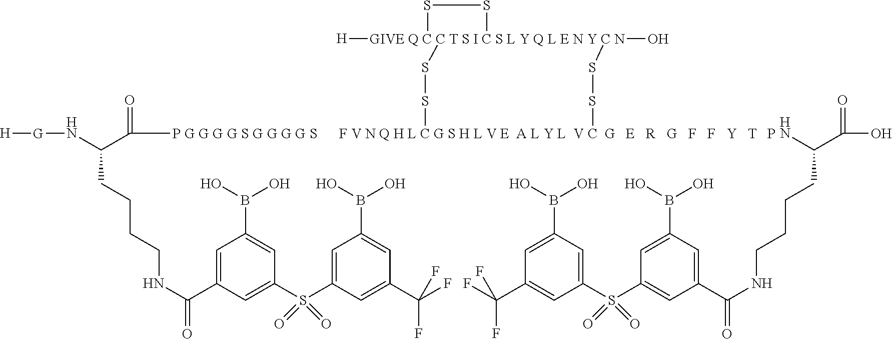

Example 288 was made similarly to example 101 from B1-GKPGGGGSGGGGS desB30 human insulin and building block of example 34.

### Example 289

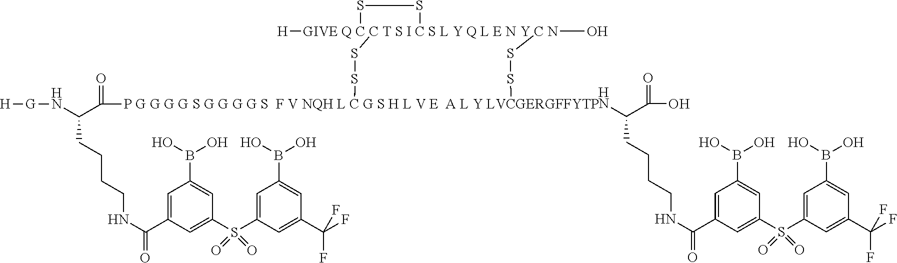

Example 289 was made similarly to example 101 from B1-GKPGGGGS desB30 human insulin and building block of example 34.

### Example 290

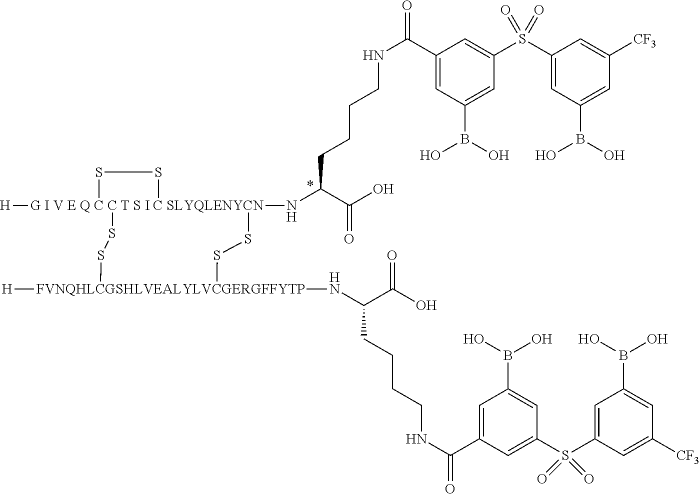

Example 290 was made similarly to example 101 from A22K desB30 human insulin and building block of example 34.

### Example 291

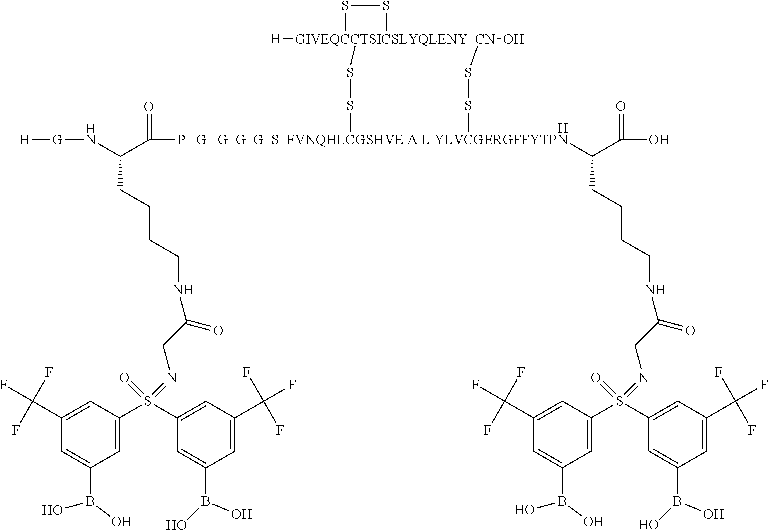

Example 291 was made similarly to example 101 from B1-GKPGGGGS desB30 human insulin and building block of example 33.

### Example 292

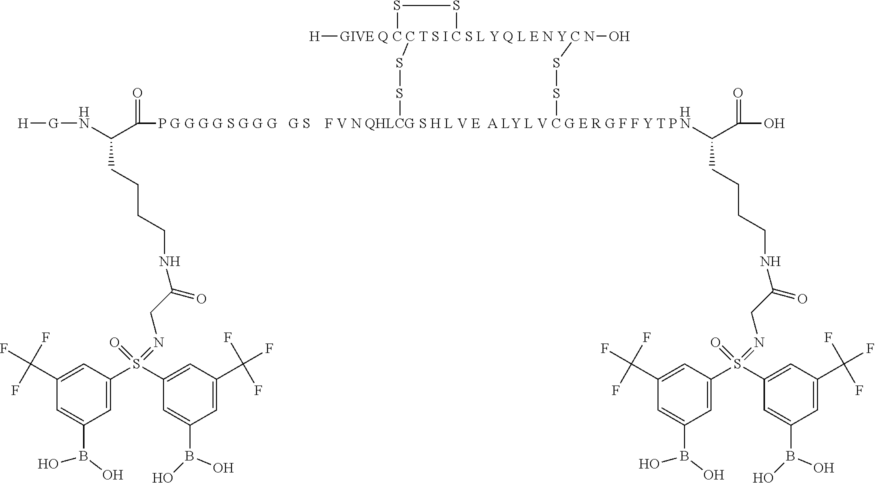

Example 292 was made similarly to example 101 from B1-GKPGGGGSGGGGS desB30 human insulin and building block of example 33.

### Example 293

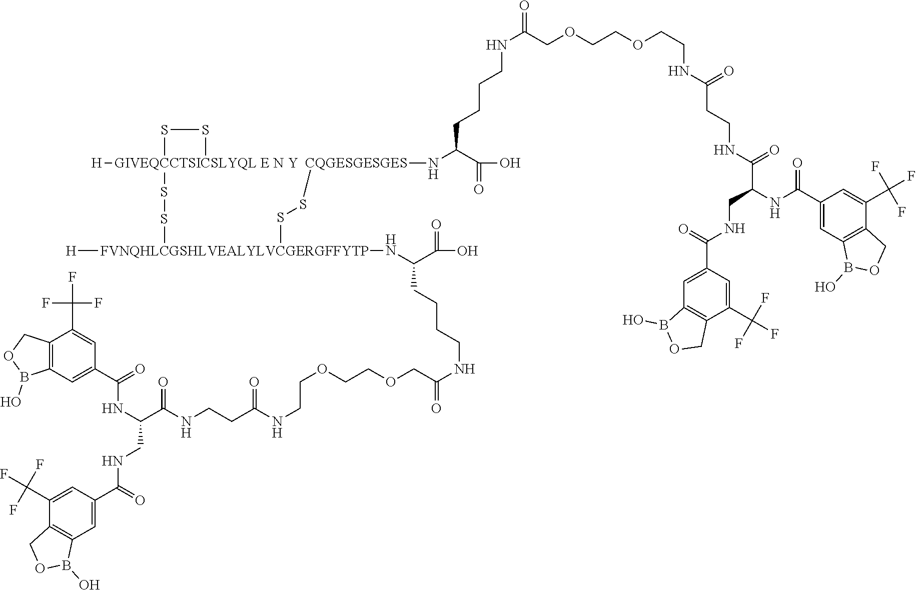

Example 293 was made by conjugation Boc-OEG to the two lysine residues of A21Q (GES)3K desB30 human insulin, similarly to conjugation of example 101, followed by removing the Boc-groups using 95% TFA, and conjugating the amino groups of OEG with building block of example 29, similar to conjugations in example 101.

### Example 294

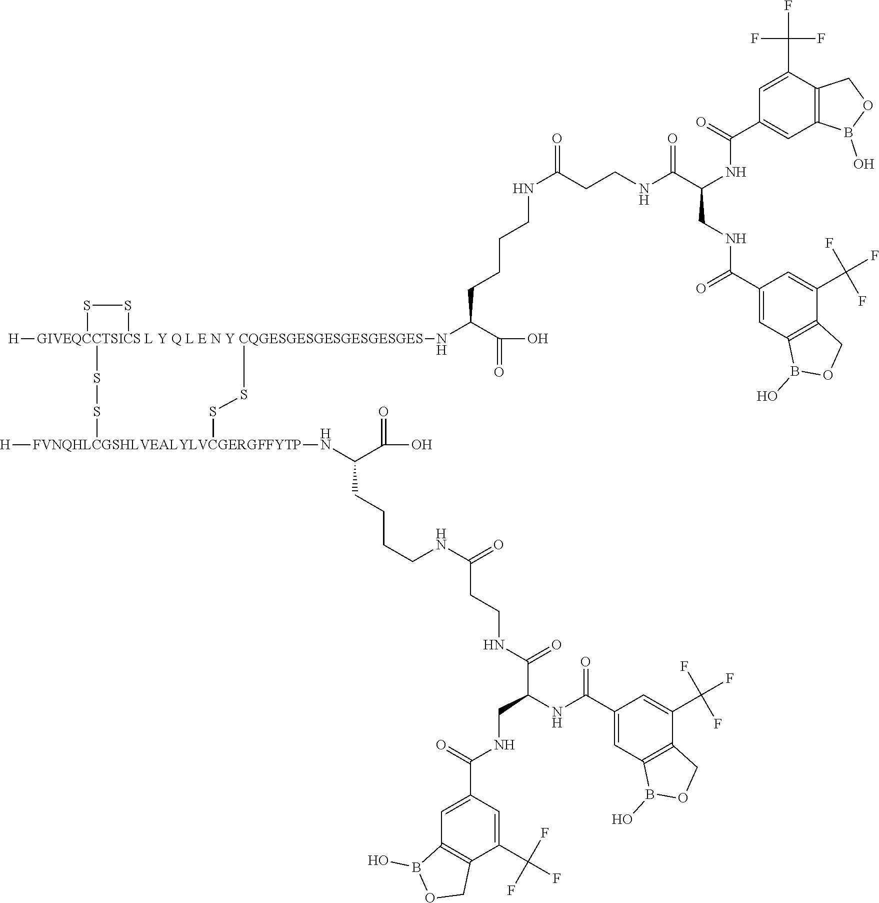

Example 294 was made similarly to example 101 from A21Q (GES)6K desB30 human insulin and building block of example 29.

### Example 295

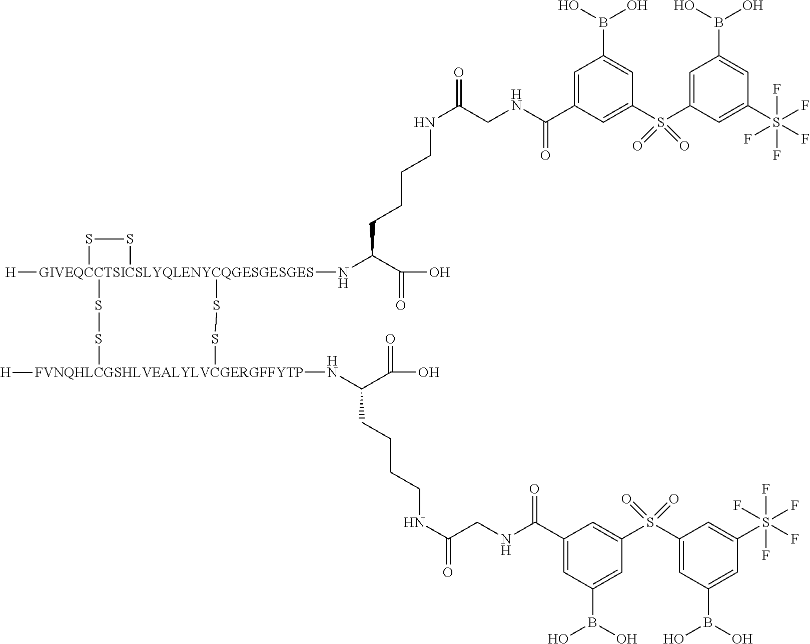

Example 295 was made similarly to example 101 from A21Q (GES)3K desB30 human insulin and building block of example 27.

### Example 296

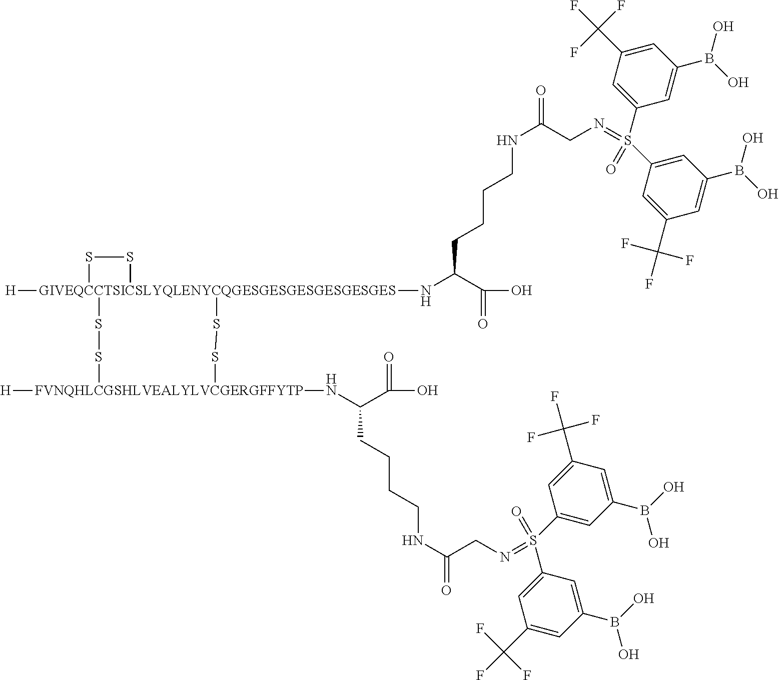

Example 296 was made similarly to example 101 from A21Q (GES)6K desB30 human insulin and building block of example 33.

### Example 297

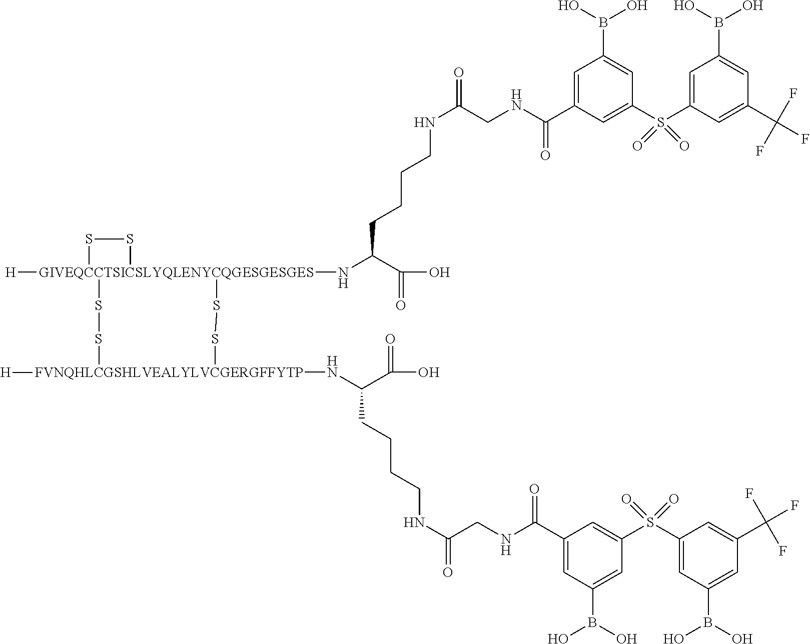

Example 297 was made similarly to example 101 from A21Q (GES)3K desB30 human insulin and building block of example 22.

### Example 298

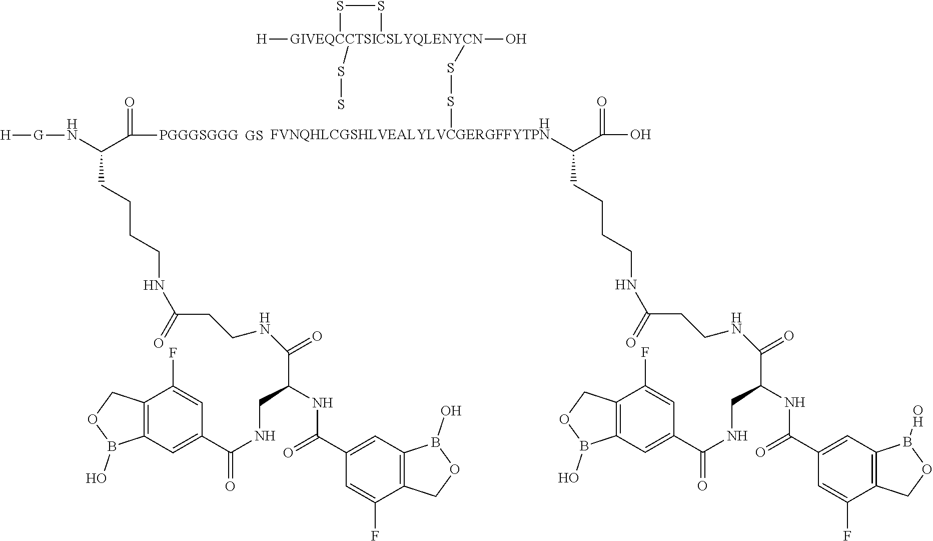

Example 298 was made similarly to example 101 from B1-GKPGGGGSGGGGS desB30 human insulin and building block of example 30.

### Example 299

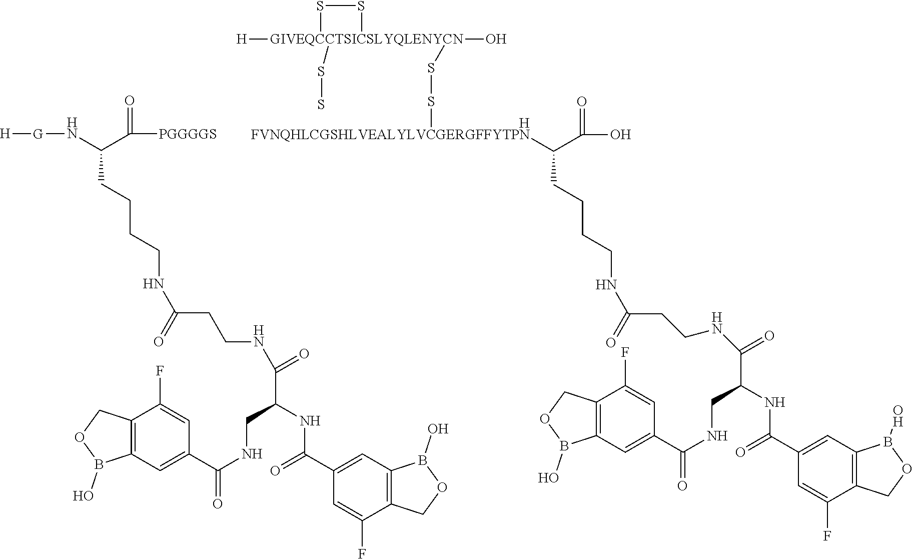

Example 299 was made similarly to example 101 from B1-GKPGGGGS desB30 human insulin and building block of example 30.

### Example 300

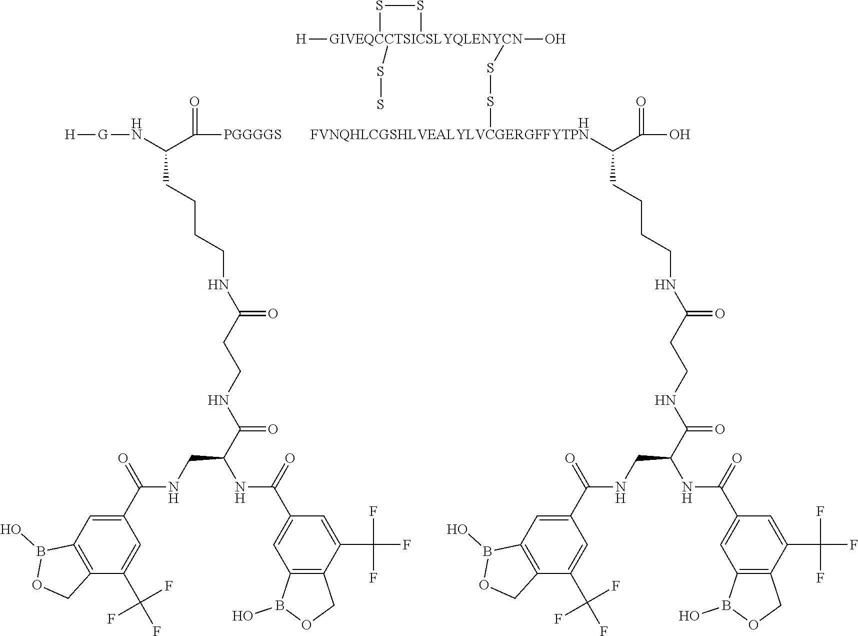

Example 300 was made similarly to example 101 from B1-GKPGGGGS desB30 human insulin and building block of example 29.

### Example 301

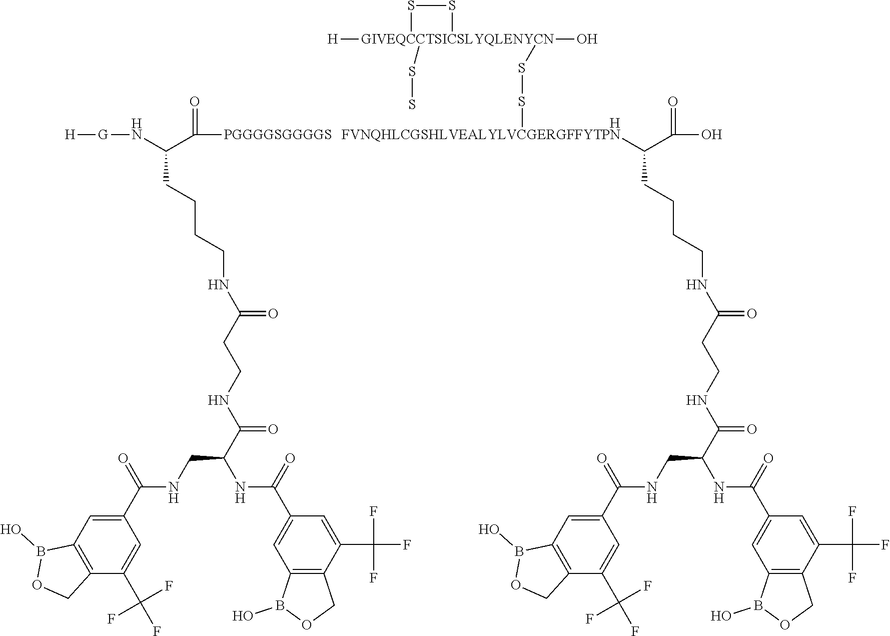

Example 301 was made similarly to example 101 from B1-GKPGGGGSGGGGS desB30 human insulin and building block of example 29.

### Example 302

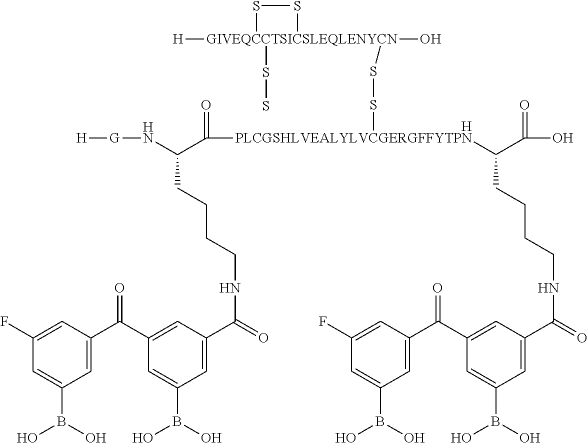

Example 302 was made similarly to example example 101 from A14E desB1-B2 B3G B4K B5P desB30 and building block of example 16.

### Example 303

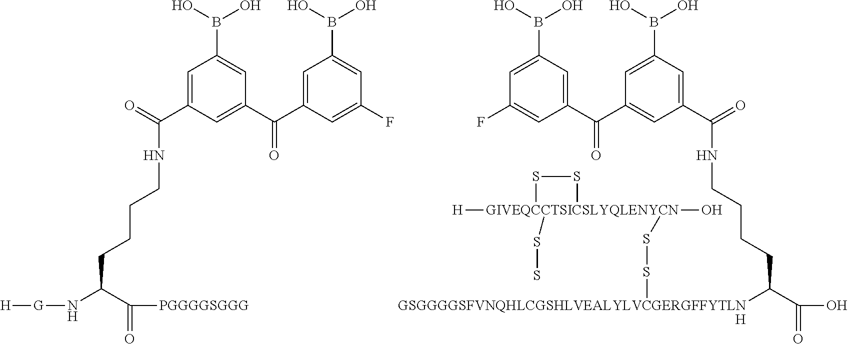

Example 303 was made similarly to example 101 from B1-GKPGGGGSGGGGSGGGGS desB30 human insulin and building block of example 16.

### Example 304

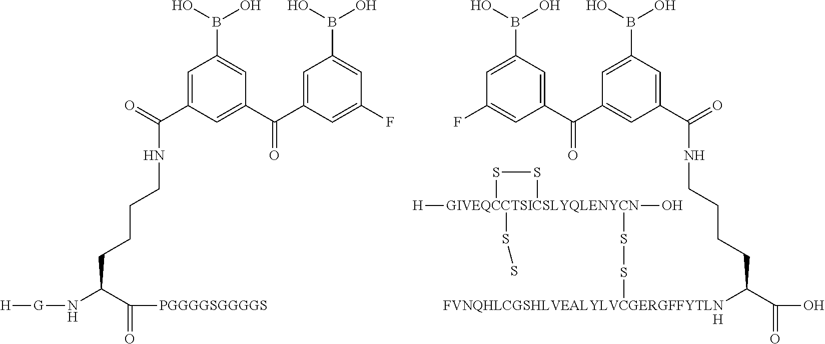

Example 304 was made similarly to example 101 from B1-GKPGGGGSGGGGS desB30 human insulin and building block of example 16.

### Example 305

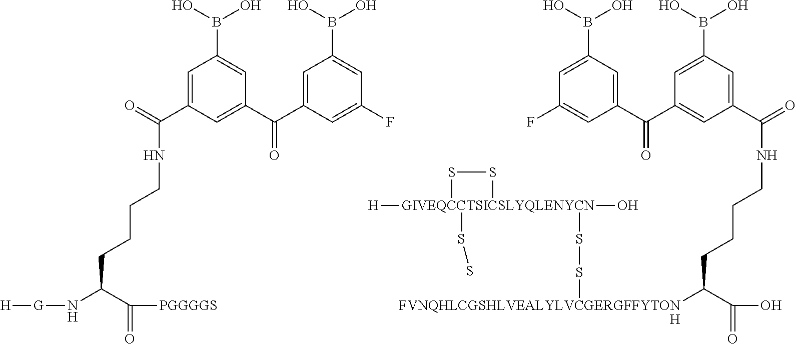

Example 305 was made similarly to example 101 from B1-GKPGGGGS desB30 human insulin and building block of example 16.

### Example 306

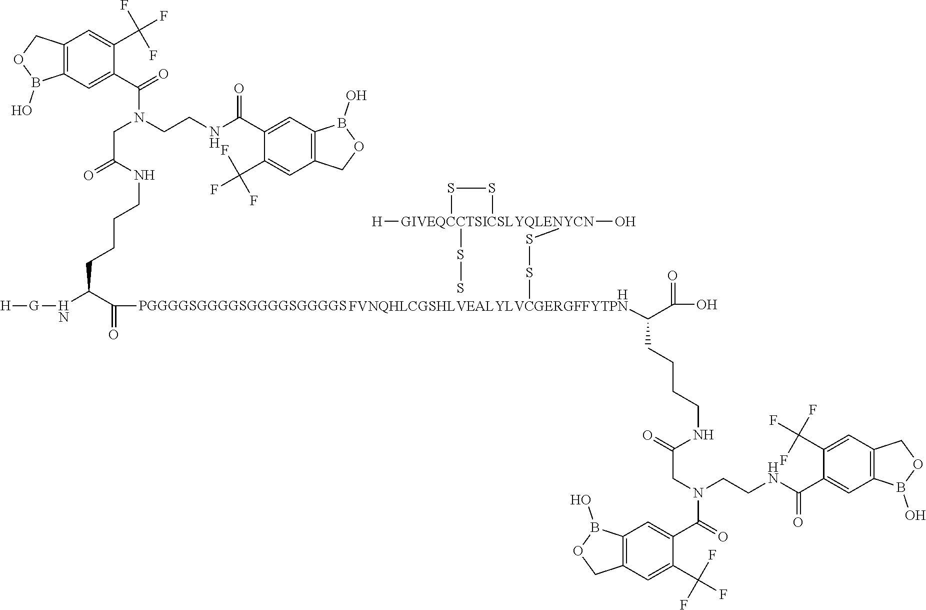

Example 306 was made similarly to example 101 from B1-GKPGGGGSGGGGSGGGGS desB30 human insulin and building block of example 35.

### Example 307

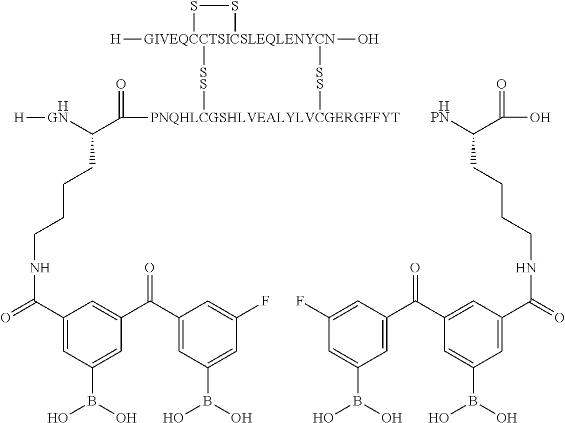

Example 307 was made similarly to example 101 from A14E B-1G B1K B2P desB30 human insulin and building block of example 16.

### Example 308

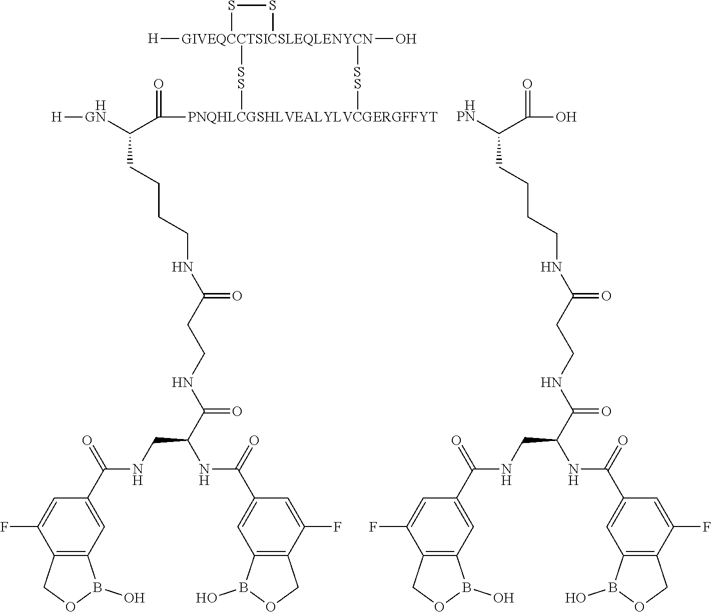

Example 308 was made similarly to example 101 from A14E B-1G B1K B2P desB30 human insulin and building block of example 30.

### Example 309

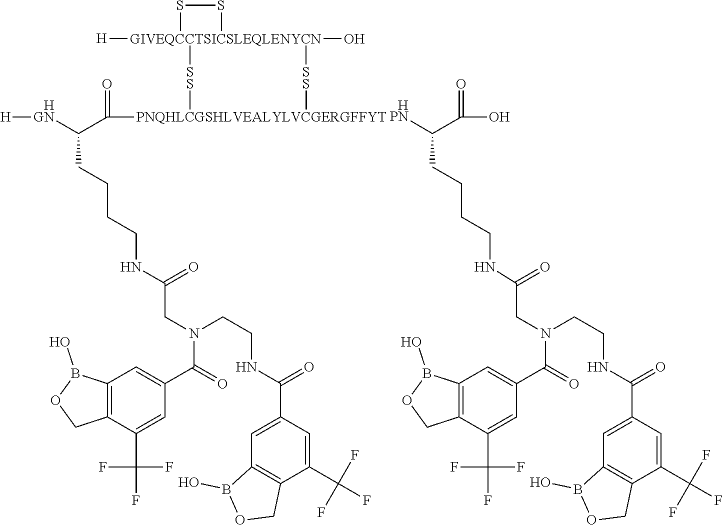

Example 309 was made similarly to example 101 from A14E B-1G B1K B2P desB30 human insulin and building block of example 28.

### Example 310

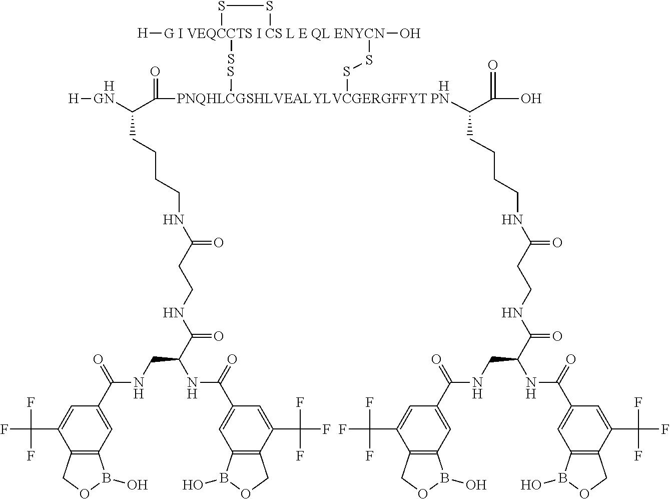

Example 310 was made similarly to example 101 from A14E B-1G B1K B2P desB30 human insulin and building block of example 29.

### Example 311

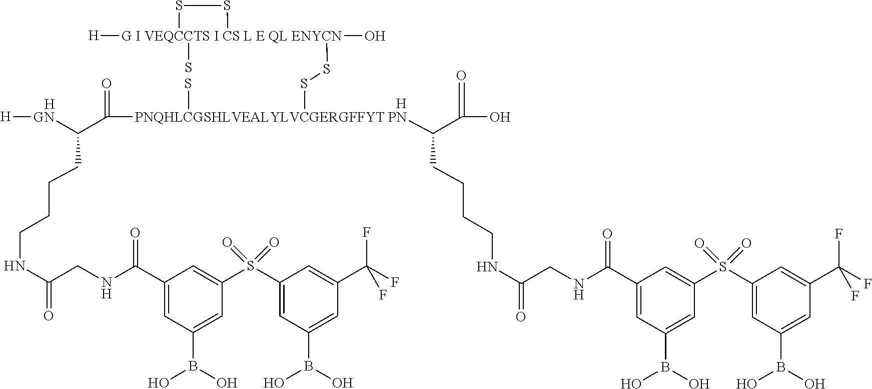

Example 311 was made similarly to example 101 from A14E B-1G B1K B2P desB30 human insulin and building block of example 22.

### Example 312

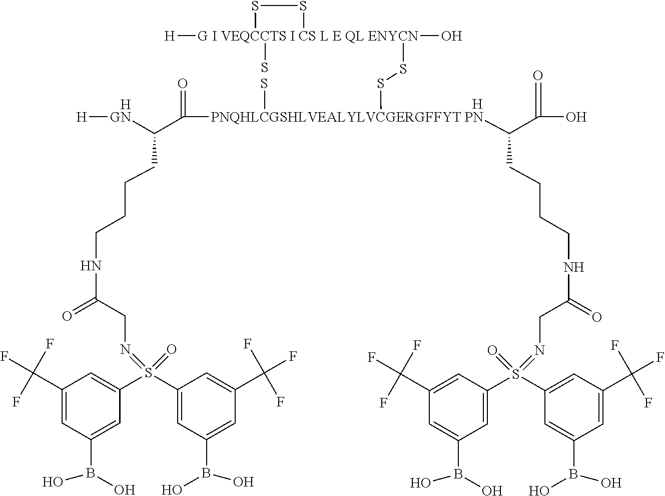

Example 312 was made similarly to example 101 from A14E B-1G B1K B2P desB30 human insulin and building block of example 16.

### Example 313

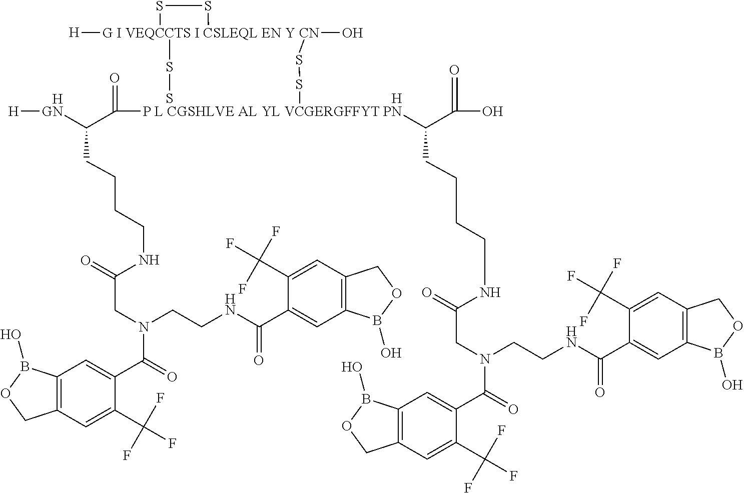

Example 313 was made similarly to example 101 from A14E desB1-B2 B3G B4K B5P desB30 human insulin and building block of example 35.

### Example 314

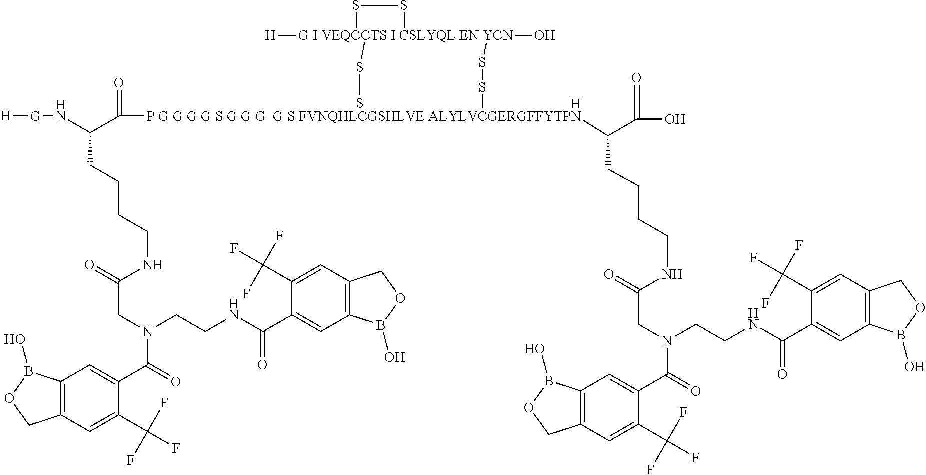

Example 314 was made similarly to example 101 from B1-GKPGGGGSGGGGS desB30 human insulin and building block of example 35.

### Example 315

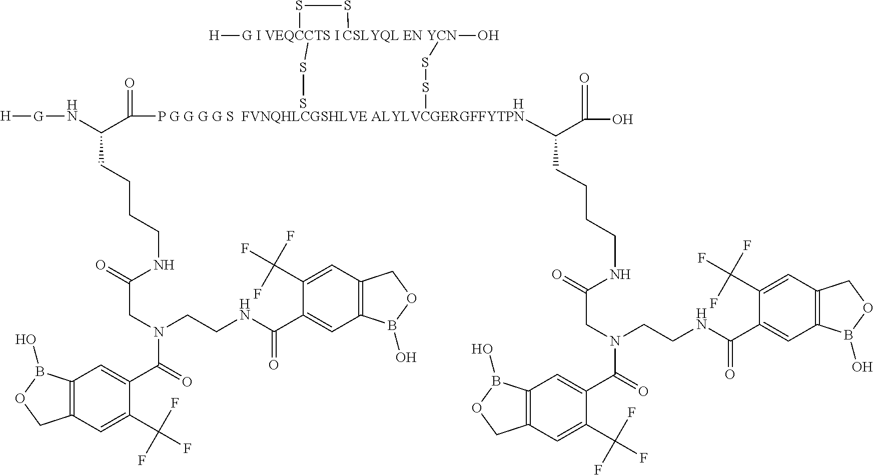

Example 315 was made similarly to example 101 from B1-GKPGGGGS desB30 human insulin and building block of example 35.

### Example 316

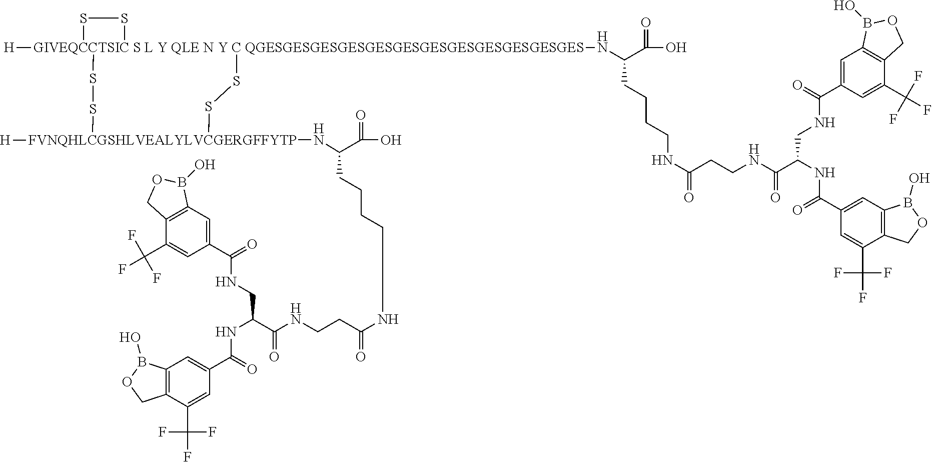

Example 316 was made similarly to example 101 from A21Q (GES)12K desB30 human insulin and building block of example 29.

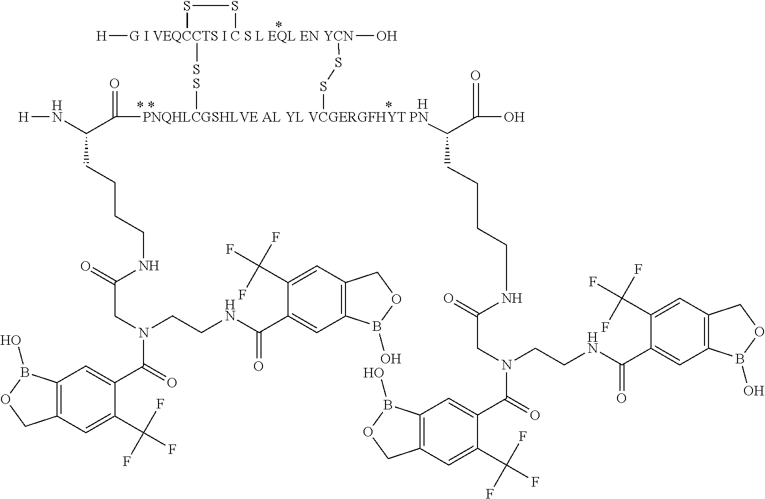

Example 317 was made similarly to example 101 from A14E B1K B2P B25H desB27 desB30 human insulin and building block of example 35.

### Example 318

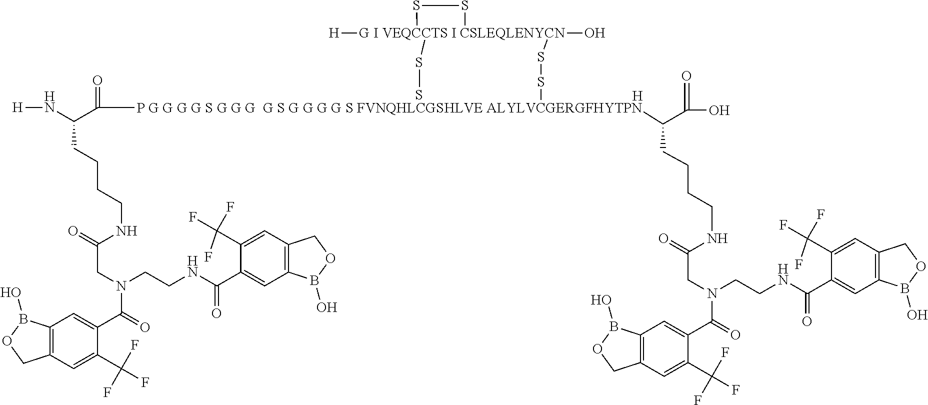

Example 318 was made similarly to example 101 from B1-KPGGGGSGGGGSGGGGS A14E B25H desB30 human insulin and building block of example 35.

### Example 319

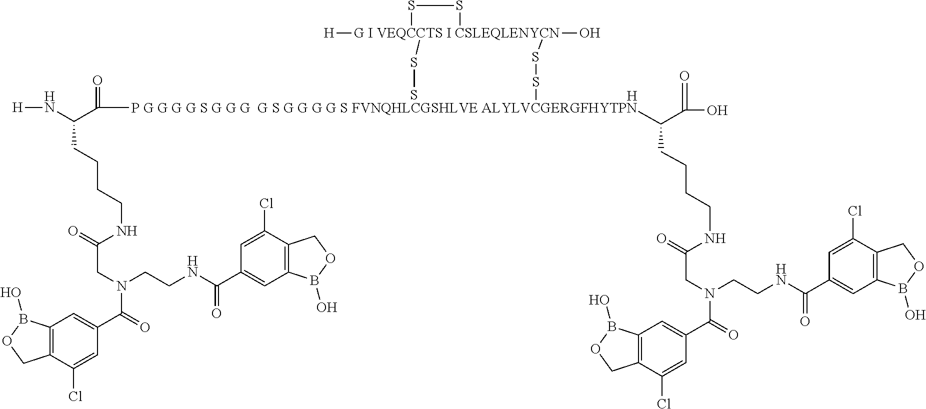

Example 319 was made similarly to example 101 from B1-KPGGGGSGGGGSGGGGS A14E B25H desB30 human insulin and building block of example 36.

### Example 320

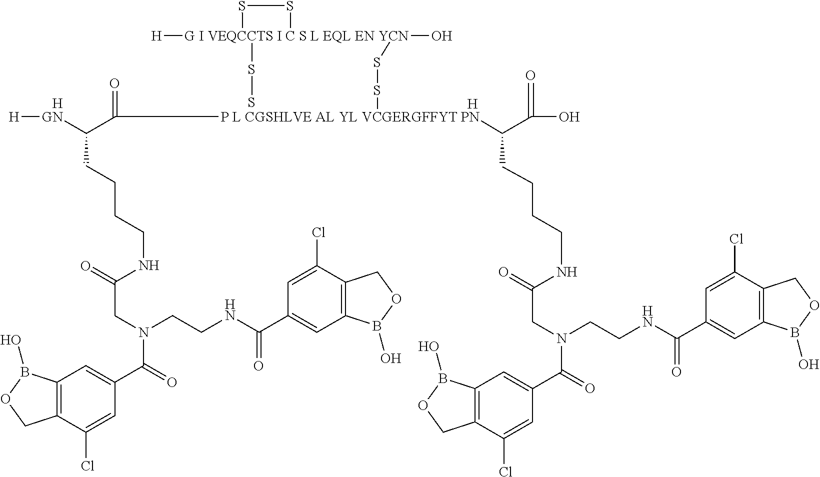

Example 320 was made similarly to example 101 from A14E desB1-B2 B3G B4K B5P desB30 human insulin and building block of example 36.

### Example 321

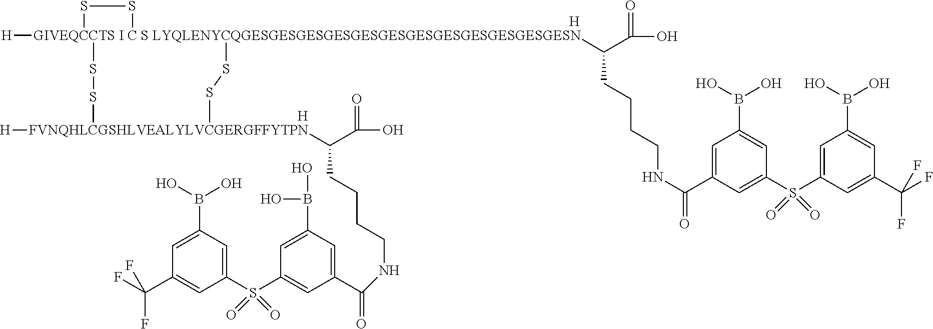

Example 321 was made similarly to example 101 from A21Q (GES)12K desB30 human insulin and building block of example 34.

### Example 322

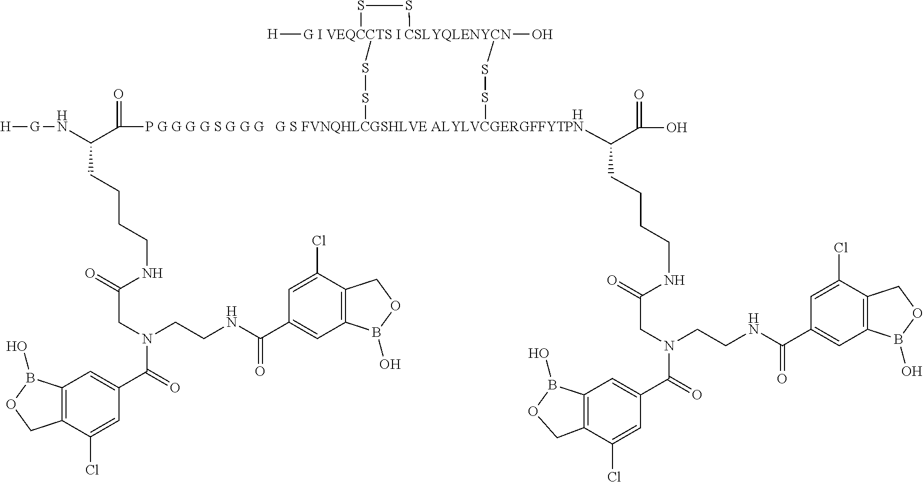

Example 322 was made similarly to example 101 from B1-GKPGGGGSGGGGS desB30 human insulin and building block of example 36.

### Example 323

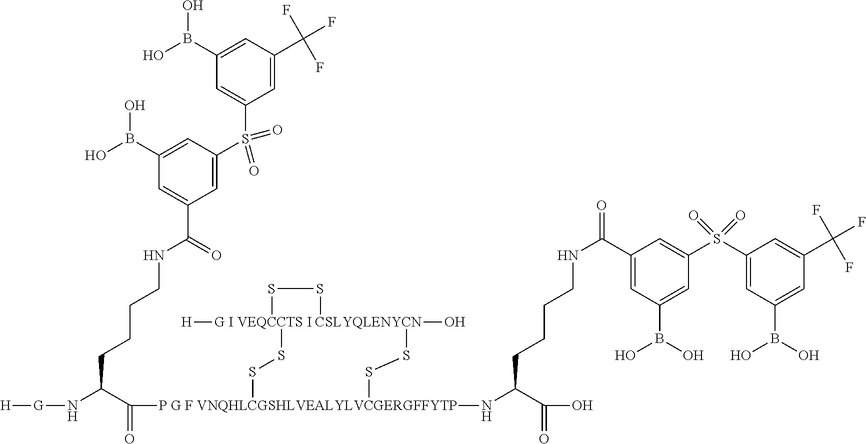

Example 323 was made similarly to example 101 from B1-GKPG desB30 human insulin and building block of example 34.

### Example 324

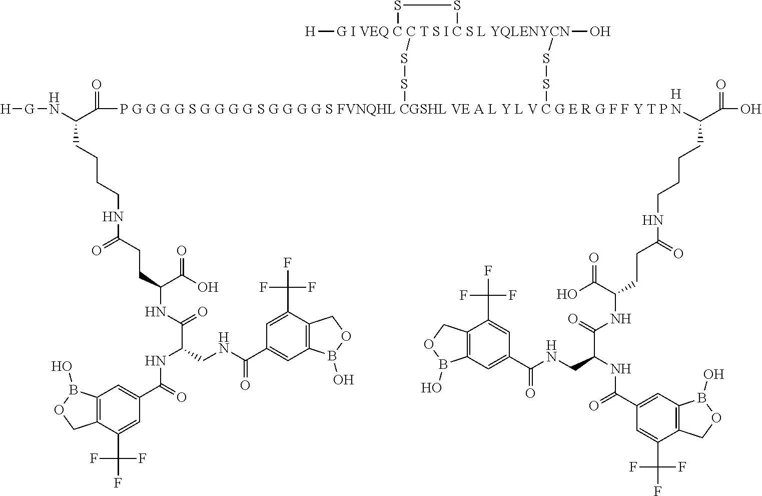

Example 324 was made similarly to example 101 from B1-GKPGGGGSGGGGSGGGGS desB30 human insulin and building block of example 37. The tert-butyl protecting group on the gamma-Glu residue of the insulin derivative was removed by treatment with 95% TFA/water for 30-60 mins at room temperature.

### Example 325

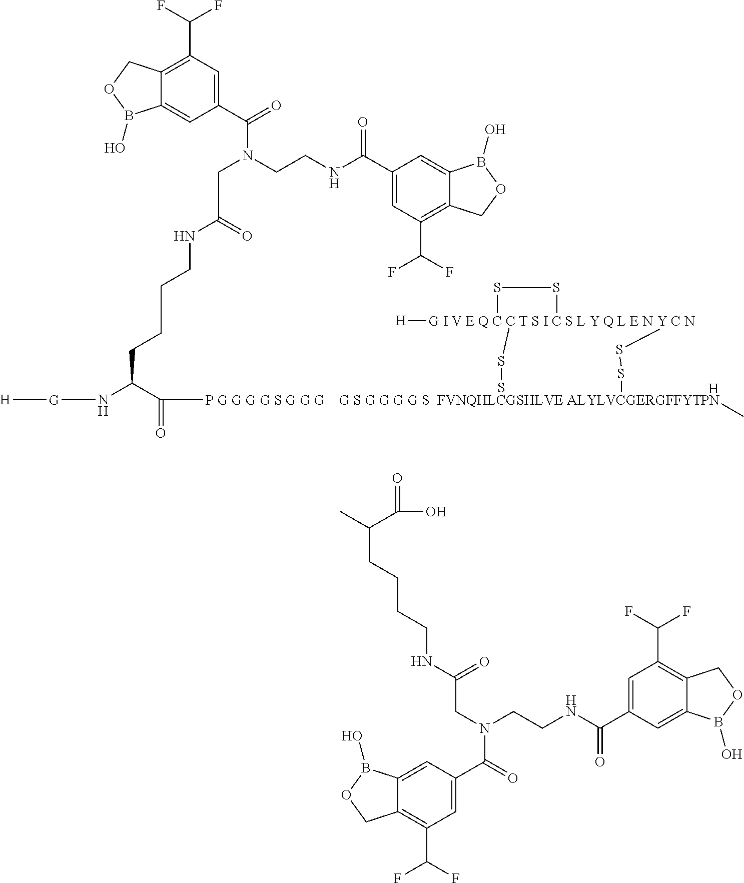

Example 325 was made similarly to example 101 from B1-GKPGGGGSGGGGSGGGGS desB30 human insulin and building block of example 38.

### Example 326

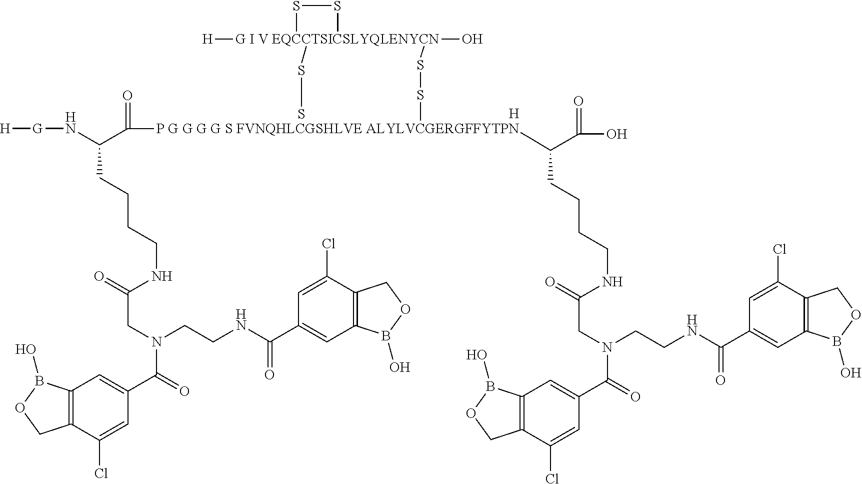

Example 326 was made similarly to example 101 from B1-GKPGGGGS desB30 human insulin and building block of example 36.

### Example 327

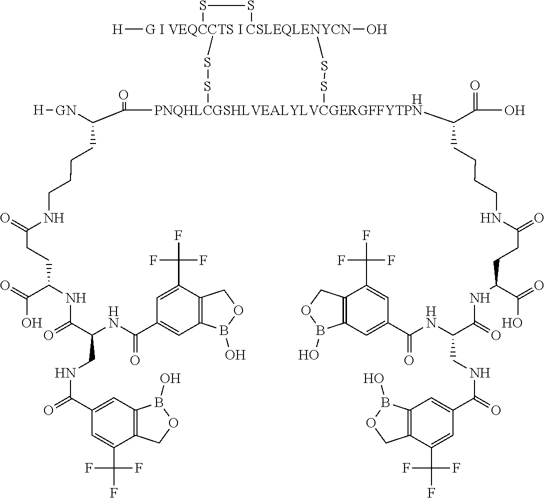

Example 327 was made similarly to example 101 from A14E B-1G B1K B2P desB30 human insulin and building block of example 37. The tert-butyl protecting group on the gamma-Glu residue of the insulin derivative was removed by treatment with 95% TFA/water for 30-60 mins at room temperature.

### Example 328

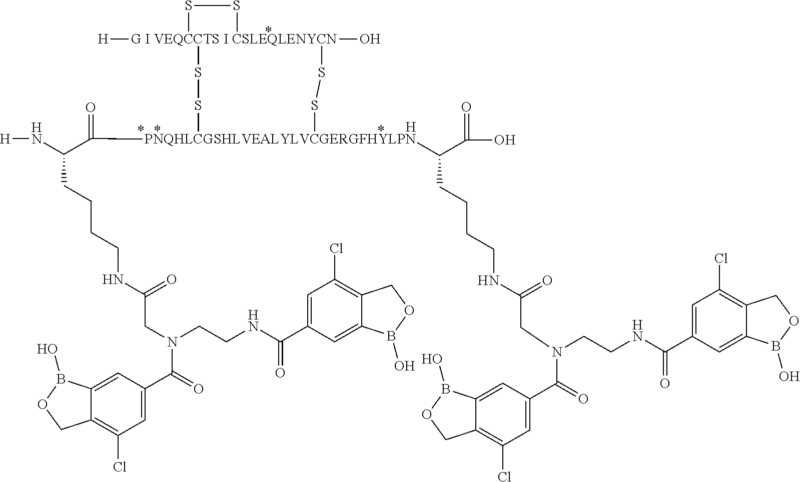

Example 328 was made similarly to example 101 from A14E B1K B2P B25H desB27 desB30 human insulin and building block of example 36.

### Example 329

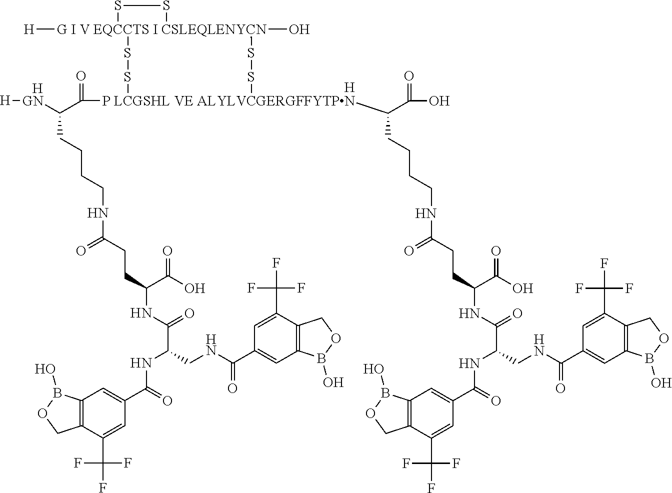

Example 329 was made similarly to example 101 from A14E desB1-B2 B3G B4K B5P desB30 human insulin and building block of example 37. The tert-butyl protecting group on the gamma-Glu residue of the insulin derivative was removed by treatment with 95% TFA/water for 30-60 mins at room temperature.

### Example 330

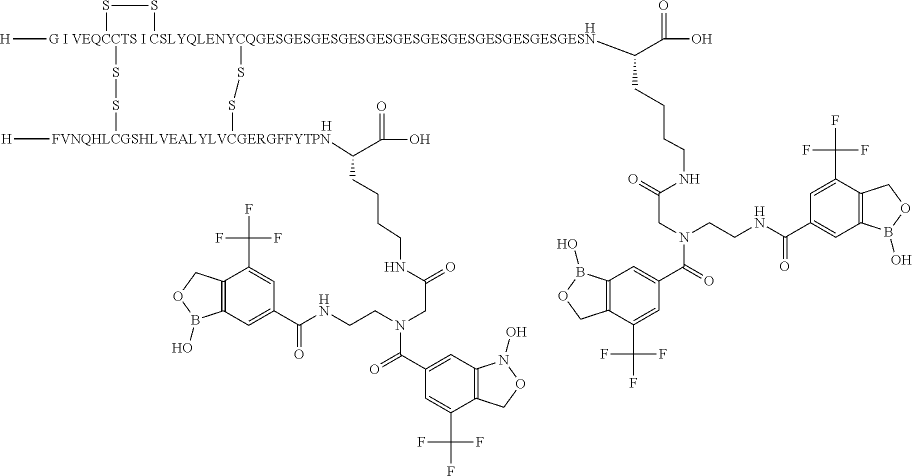

Example 330 was made similarly to example 101 from A21Q (GES)12K desB30 human insulin and building block of example 28.

### Example 331

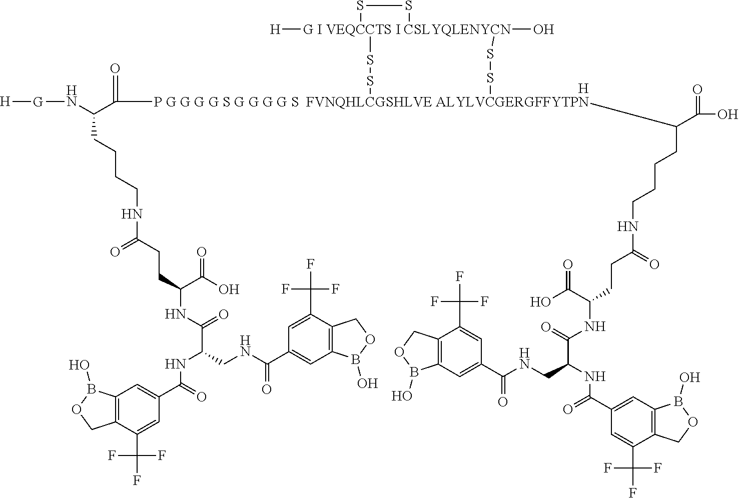

Example 331 was made similarly to example 101 from B1-GKPGGGGSGGGGS desB30 human insulin and building block of example 37. The tert-butyl protecting group on the gamma-Glu residue of the insulin derivative was removed by treatment with 95% TFA/water for 30-60 mins at room temperature.

### Example 332

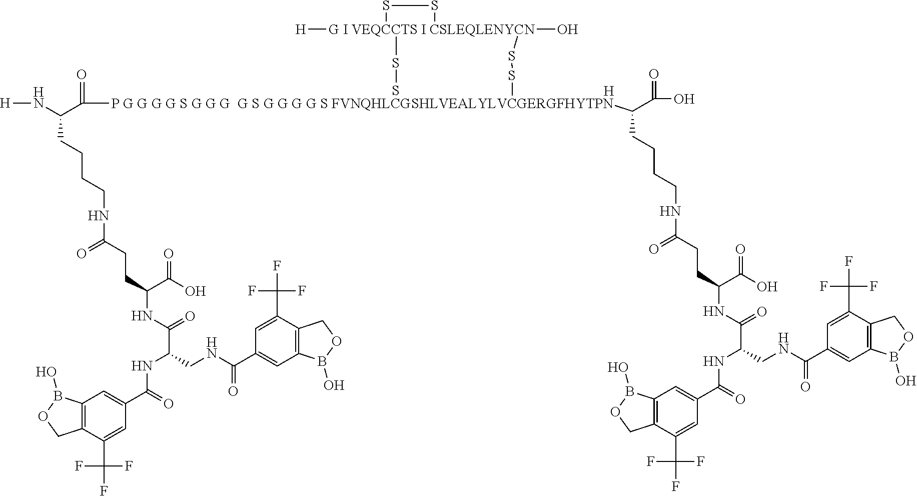

Example 332 was made similarly to example 101 from B1-KPGGGGSGGGGSGGGGS A14E B25H desB30 human insulin and building block of example 37. The tert-butyl protecting group on the gamma-Glu residue of the insulin derivative was removed by treatment with 95% TFA/water for 30-60 mins at room temperature.

### Example 333

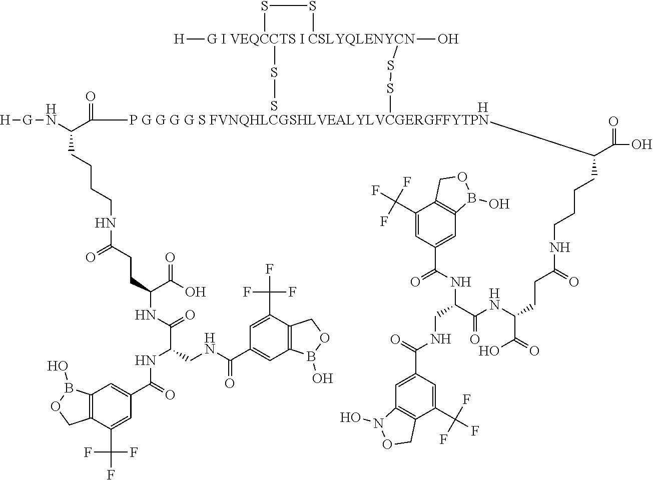

Example 333 was made similarly to example 101 from B1-GKPGGGGS desB30 human insulin and building block of example 37. The tert-butyl protecting group on the gamma-Glu residue of the insulin derivative was removed by treatment with 95% TFA/water for 30-60 mins at room temperature.

### Example 334

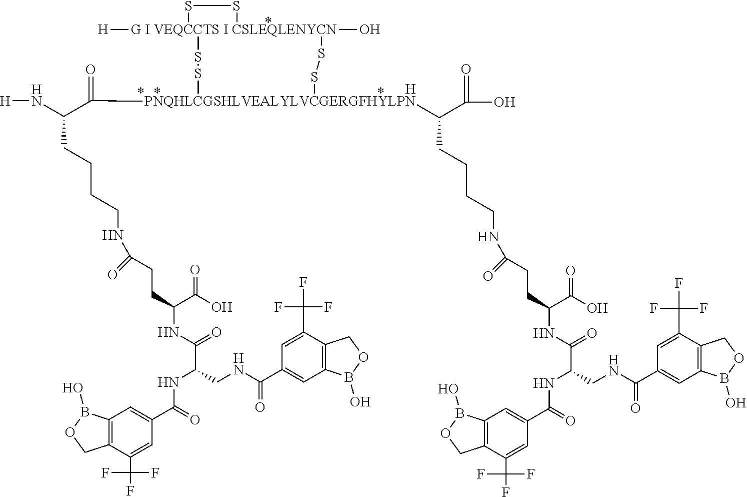

Example 334 was made similarly to example 101 from A14E B1K B2P B25H desB27 desB30 human insulin and building block of example 37. The tert-butyl protecting group on the gamma-Glu residue of the insulin derivative was removed by treatment with 95% TFA/water for 30-60 mins at room temperature.

### Example 335

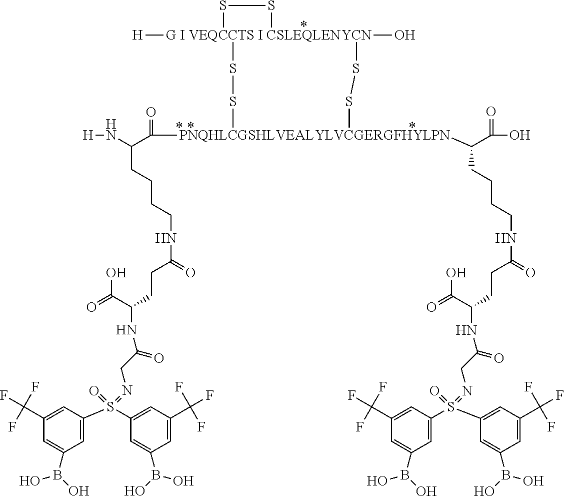

Example 335 was made similarly to example 101 from A14E B1K B2P B25H desB27 desB30 human insulin and building block of example 39. The tert-butyl protecting group on the gamma-Glu residue of the insulin derivative was removed by treatment with 95% TFA/water for 30-60 mins at room temperature.

### Example 336

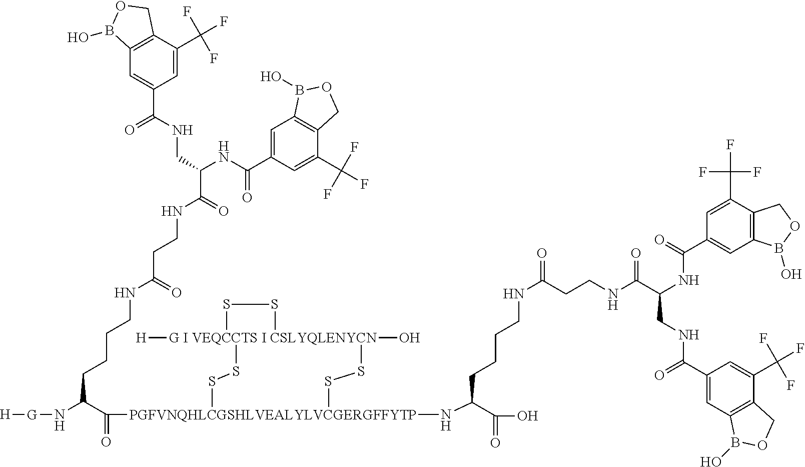

Example 336 was made similarly to example 101 from B1-GKPG desB30 human insulin and building block of example 29.

### Example 337

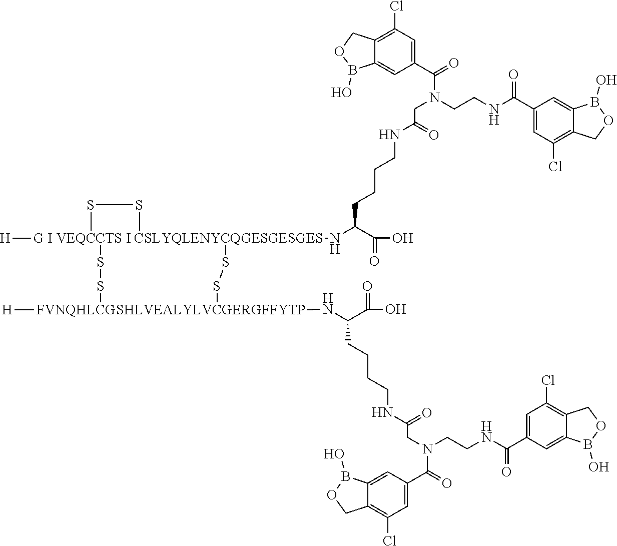

Example 337 was made similarly to example 101 from A21Q (GES)3K desB30 human insulin and building block of example 36.

### Example 338

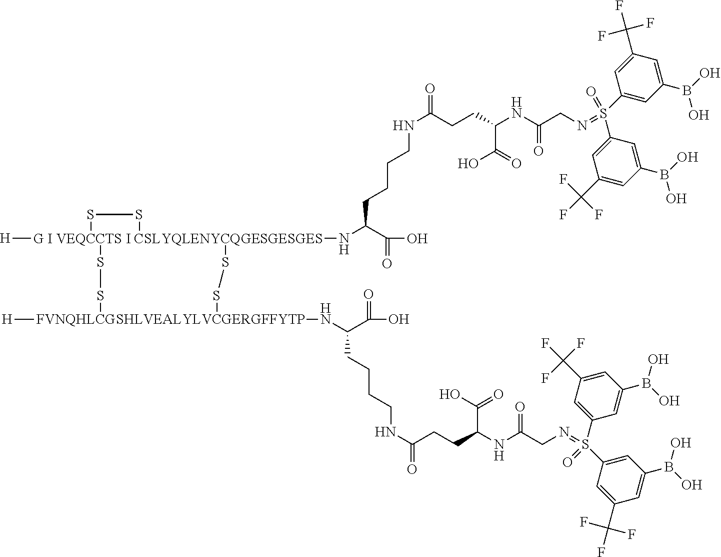

Example 338 was made similarly to example 101 from A21Q (GES)3K desB30 human insulin and building block of example 39. The tert-butyl protecting group on the gamma-Glu residue of the insulin derivative was removed by treatment with 95% TFA/water for 30-60 mins at room temperature.

### Example 339

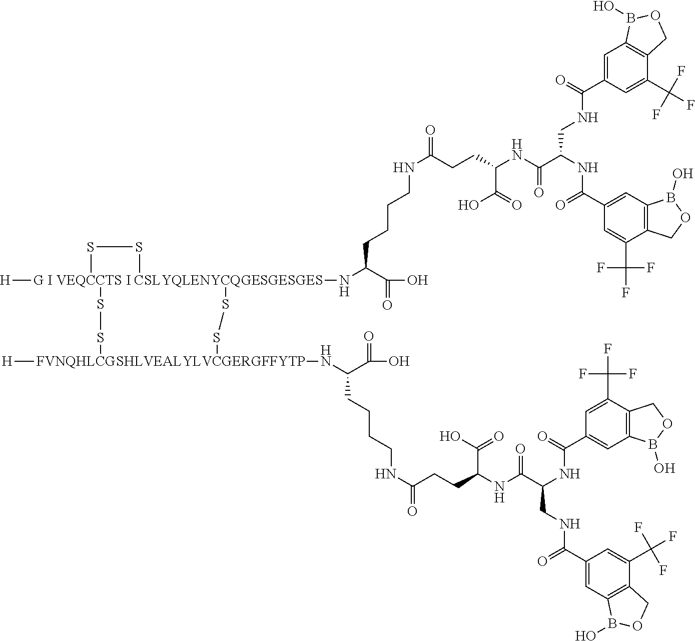

Example 339 was made similarly to example 101 from A21Q (GES)3K desB30 human insulin and building block of example 37. The tert-butyl protecting group on the gamma-Glu residue of the insulin derivative was removed by treatment with 95% TFA/water for 30-60 mins at room temperature.

### Example 340

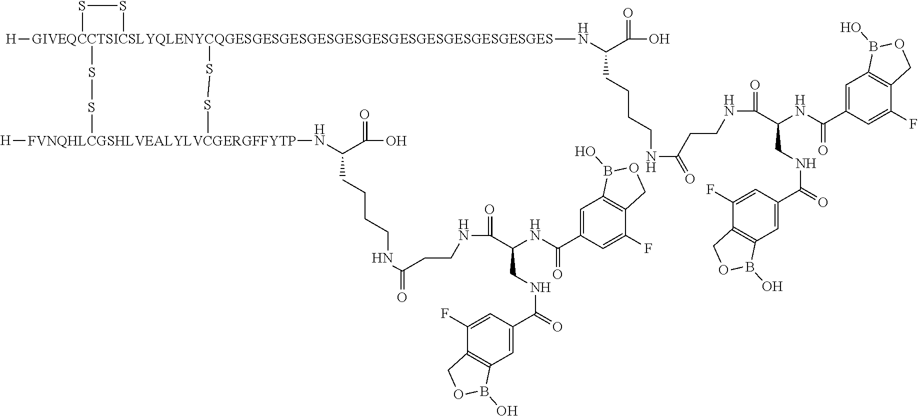

Example 340 was made similarly to example 101 from A21Q (GES)12K desB30 human insulin and building block of example 30.

### Example 341

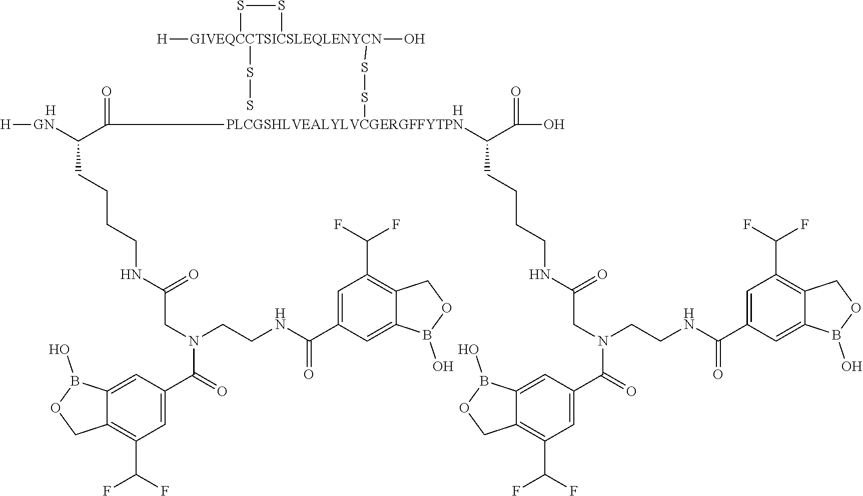

Example 341 was made similarly to example 101 from A21Q (GES)12K desB30 human insulin and building block of example 38.

### Example 342

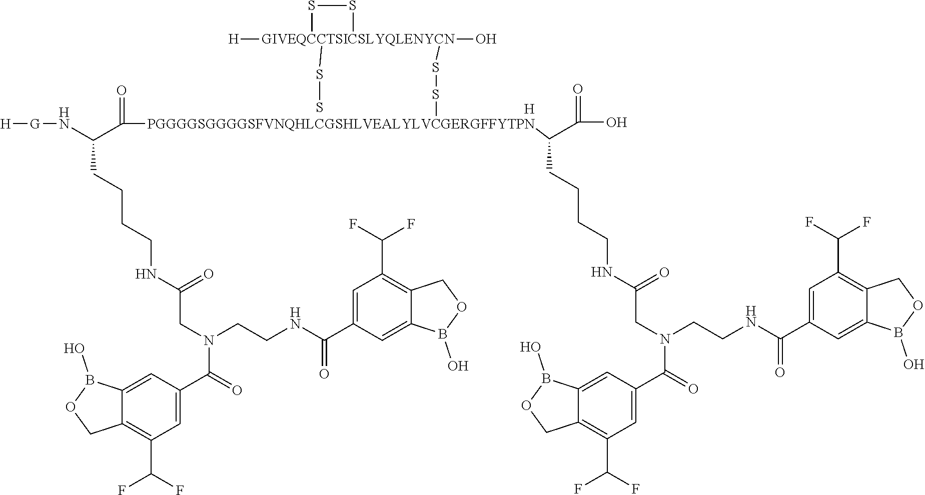

Example 342 was made similarly to example 101 from B1-GKPGGGGSGGGGS desB30 human insulin and building block of example 38.

### Example 343

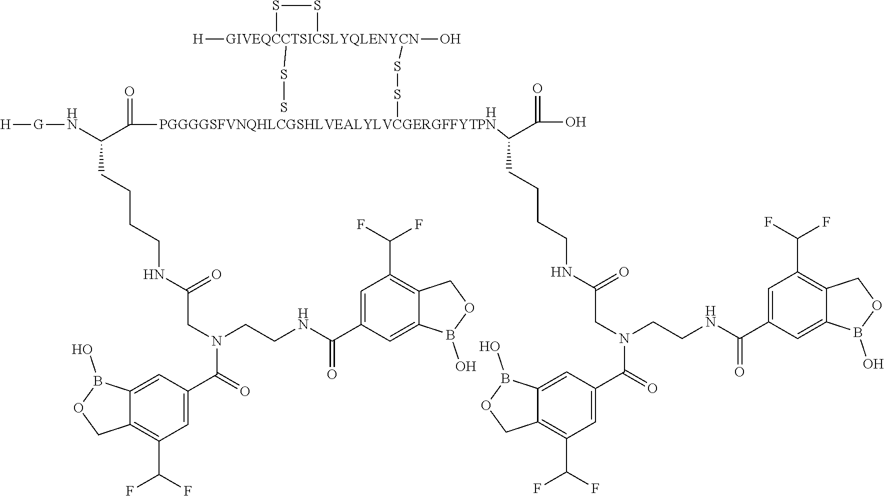

Example 343 was made similarly to example 101 from B1-GKPGGGGS desB30 human insulin and building block of example 38.

### Example 344

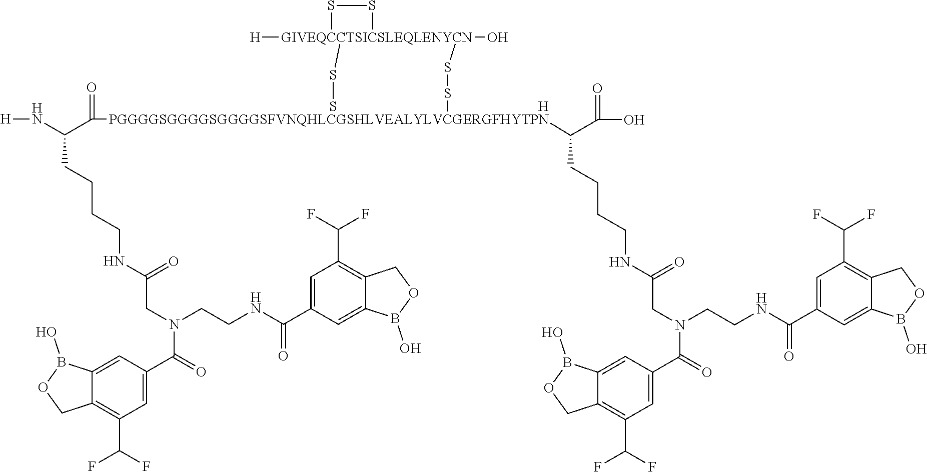

Example 344 was made similarly to example 101 from B1-KPGGGGSGGGGSGGGGS A14E B25H desB30 human insulin and building block of example 38.

### Example 354

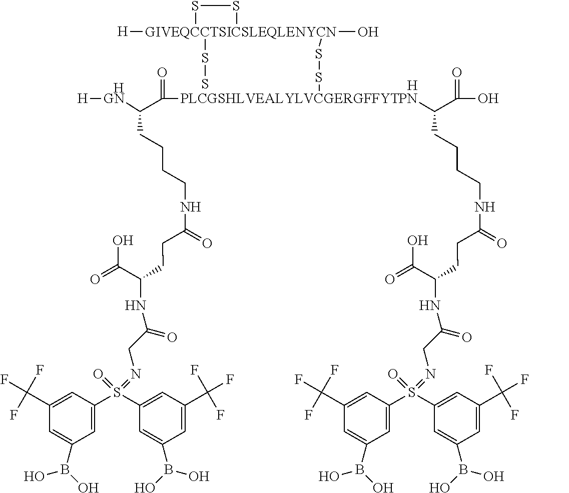

Example 345 was made similarly to example 101 from A14E desB1-B2 B3G B4K B5P desB30 human insulin and building block of example 39. The tert-butyl protecting group on the gamma-Glu residue of the insulin derivative was removed by treatment with 95% TFA/water for 30-60 mins at room temperature.

### Example 346

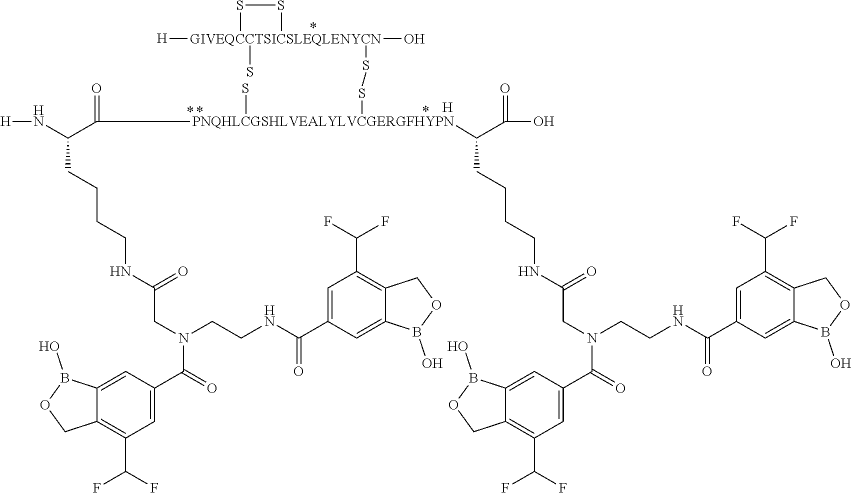

Example 346 was made similarly to example 101 from A14E B1K B2P B25H desB27 desB30 human insulin and building block of example 38.

### Example 347

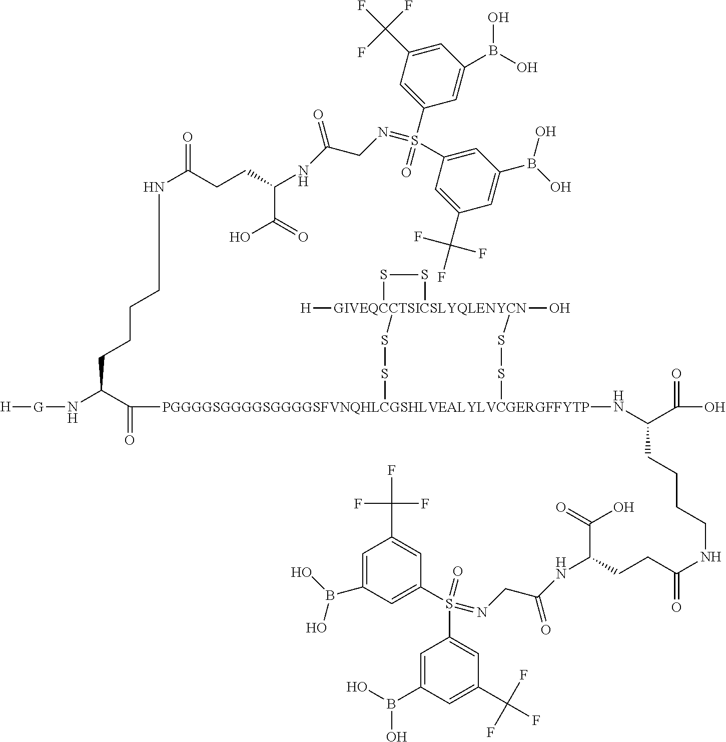

Example 347 was made similarly to example 101 from B1-GKPGGGGSGGGGSGGGGS desB30 human insulin and building block of example 39. The tert-butyl protecting group on the gamma-Glu residue of the insulin derivative was removed by treatment with 95% TFA/water for 30-60 mins at room temperature.

### Example 348

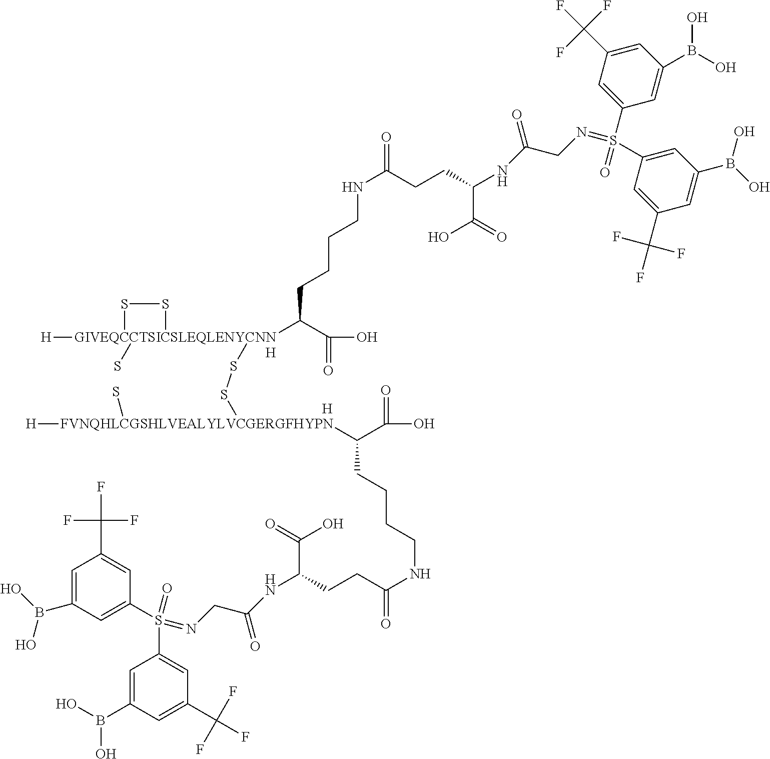

Example 348 was made similarly to example 101 from A14E A22K B25H desB27 desB30 human insulin and building block of example 39. The tert-butyl protecting group on the gamma-Glu residue of the insulin derivative was removed by treatment with 95% TFA/water for 30-60 mins at room temperature.

### Example 349

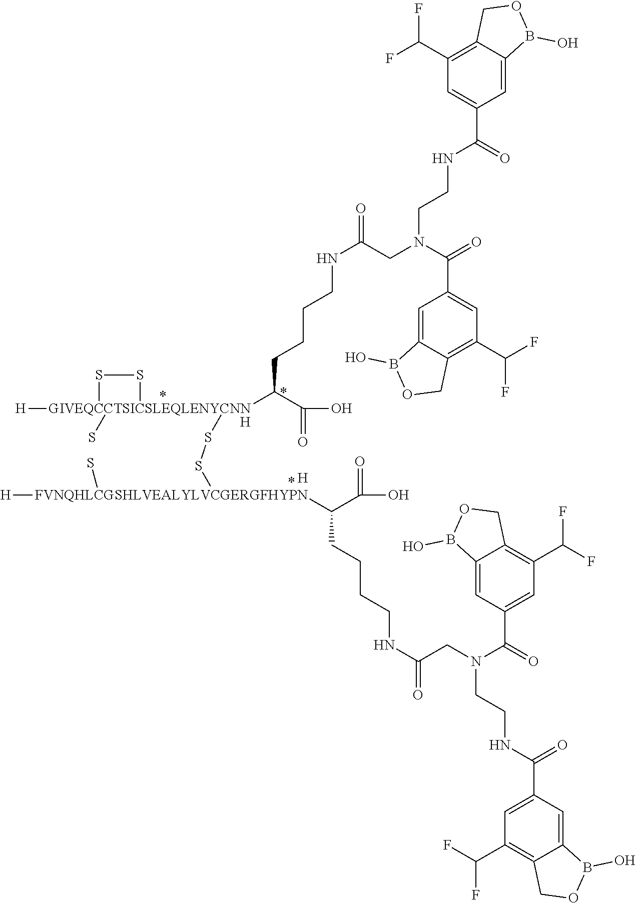

Example 349 was made similarly to example 101 from A14E A22K B25H desB27 desB30 human insulin and building block of example 38.

### Example 350

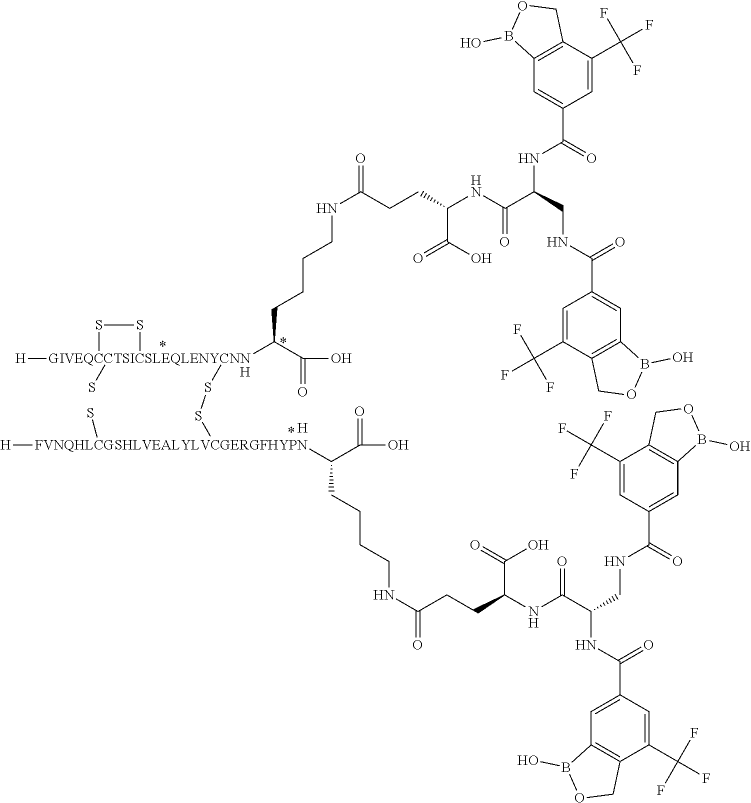

Example 350 was made similarly to example 101 from A14E A22K B25H desB27 desB30 human insulin and building block of example 37. The tert-butyl protecting group on the gamma-Glu residue of the insulin derivative was removed by treatment with 95% TFA/water for 30-60 mins at room temperature.

### Example 351

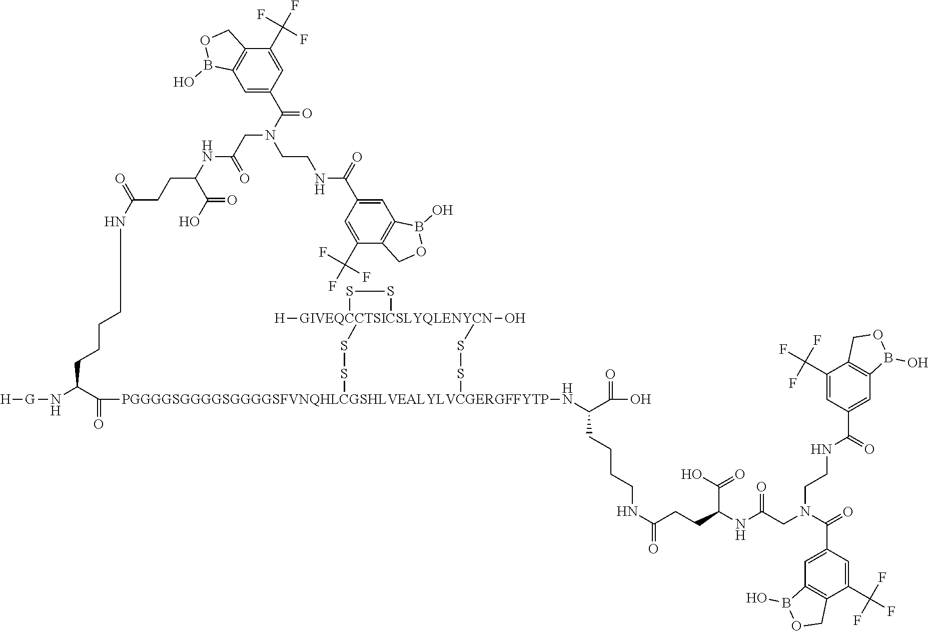

Example 351 was made similarly to example 101 from B1-GKPGGGGSGGGGSGGGGS desB30 human insulin and building block of example 40. The tert-butyl protecting group on the gamma-Glu residue of the insulin derivative was removed by treatment with 95% TFA/water for 30-60 mins at room temperature.

### Example 352

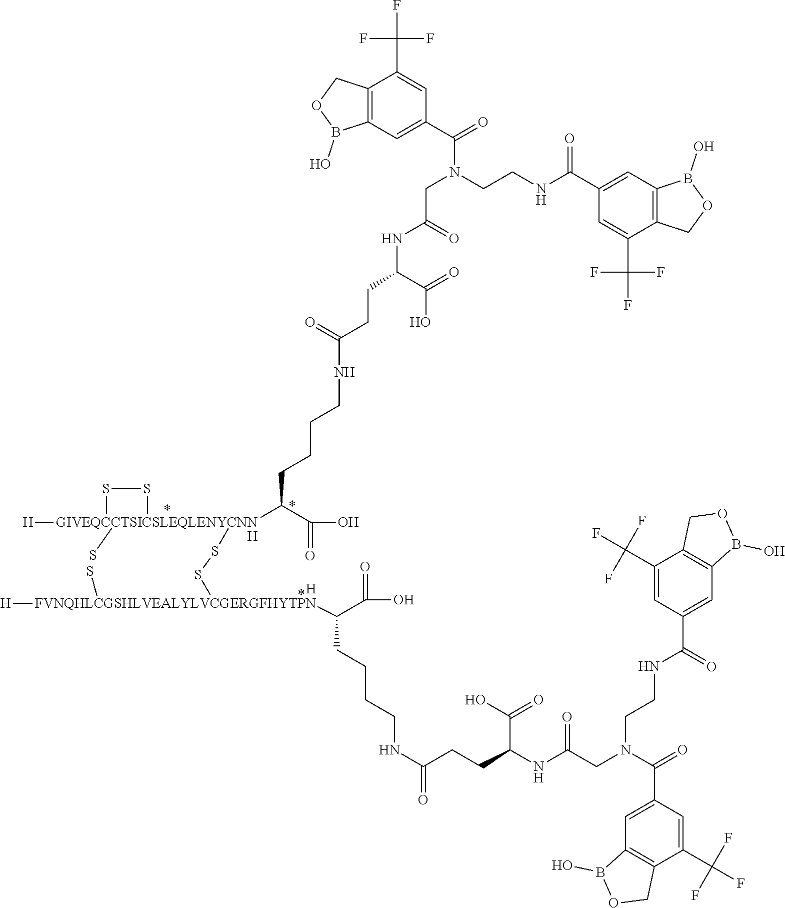

Example 352 was made similarly to example 101 from A14E A22K B25H desB27 desB30 human insulin and building block of example 40. The tert-butyl protecting group on the gamma-Glu residue of the insulin derivative was removed by treatment with 95% TFA/water for 30-60 mins at room temperature.

### Example 353

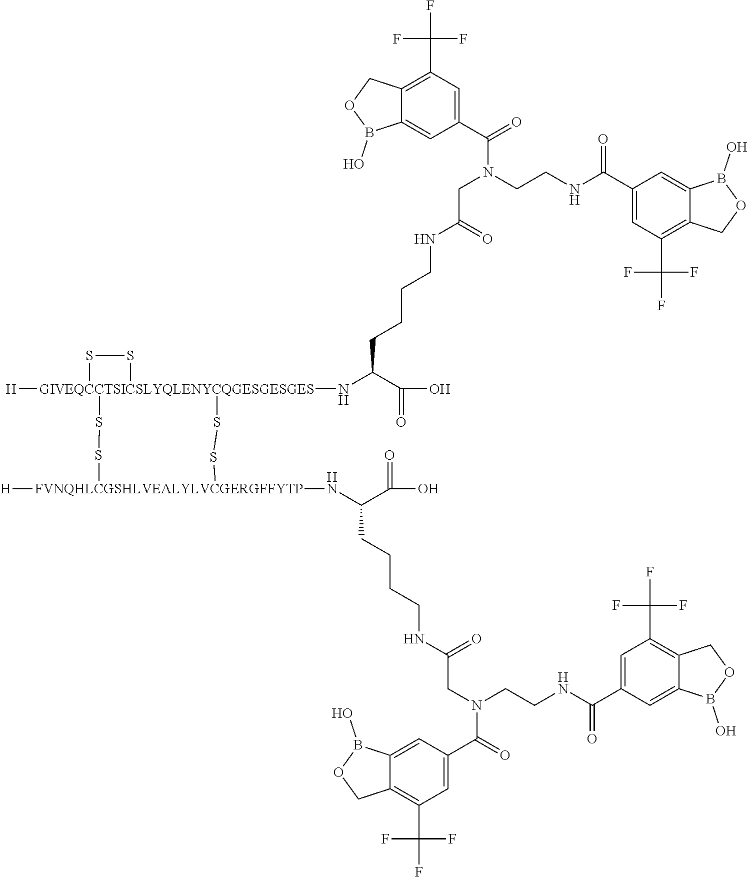

Example 353 was made similarly to example 101 from A21Q (GES)3K desB30 human insulin and building block of example 38.

### Example 354

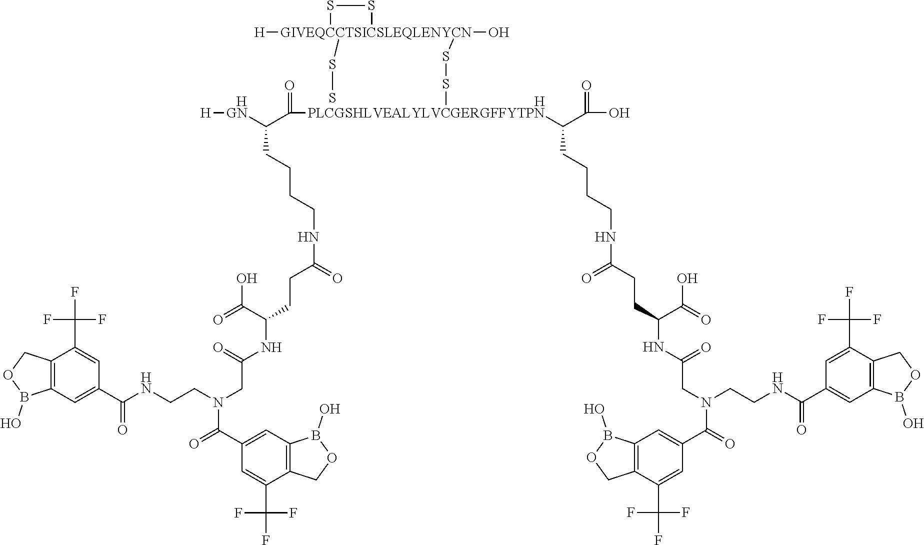

Example 354 was made similarly to example 101 from A14E desB1-B2 B3G B4K B5P desB30 human insulin and building block of example 40. The tert-butyl protecting group on the gamma-Glu residue of the insulin derivative was removed by treatment with 95% TFA/water for 30-60 mins at room temperature.

### Example A: Carbohydrates and Diboronate Binding Affinity, Alizarin Assay (ARS)

The alizarin-red binding assay is a colorimetric assay used to determine the inhibition affinity of boronate/boroxole compounds to glucose. The assay is based on a colour shift of alizarin-red upon binding to boronate, which shift can be followed by change in absorbance in the 330-340 nm region.

Determination of the Dissociation Constant (K_{d}) of Boron Compounds Towards Alizarin

For determination of the dissociation constant (Kd) between the Alizarin Red Sodium (ARS) and the boronate compound, 200 μM of ARS is dissolved in a 20 mM of phosphate buffer pH 7.4, and titrated in triplicate into a 96 well plate with 1, 0.5, 0.25, 0.125, 62.5, 31.25, 15.625, 7.812, 3.906, 1.953, 0.9767, 0.488 and 0.244 mM of boronic acid. After 5 minutes of centrifugation at 4000 rpm, the plate is placed in a multi-well spectrometer (SpectraMax, Molecular Devices) for absorption detection.

The analysis is carried out at room temperature with absorption readings at 330, 340 and 520 nm, respectively. Data obtained for absorption versus concentration of boronate is then fitted (Prism 7, GraphPad) with a sigmoidal function to obtain the Kd value of boronate and ARS.

Determination of the Displacement Constant (K_{d}) of Glucose Towards Boron Compounds

For determination of the inhibitory constant (Ki) between the boronate and the carbohydrate, 400 μM of boronic acids is dissolved in a 20 mM phosphate buffer pH 7.4 under gentle stirring. Upon complete dissolution of the compound, 200 μM of Alizarin red (ARS) is added to the solution. The ARS-boronate solution is then aliquoted into a 96 multiwell plate (black, flat and clear bottom) 1:1 with appropriate carbohydrate. In particular, D-glucose and L-lactate solutions are prepared in a 20 mM phosphate buffer pH 7.4 at these concentrations respectively: 1000, 500, 250, 100, 50, 25, 10, 5, 2.5, 1, 0.25, 0.1 mM and 2500, 1000, 500, 100, 50, 10, 5, 1, 0.5, 0.1, 0.05, 0.01 mM. The plate with ARS-boronate mixed with carbohydrate is incubated 20 minutes at room temperature. After 5 minutes of centrifugation at 4000 rpm the plate is placed in a multiwell spectrometer (SpectraMax, Molecular Devices) for absorption detection.

The analysis is carried out at room temperature with absorption readings at 330, 340 and 520 nm, respectively. Data obtained for absorption versus concentration of carbohydrate is then fitted (Prism 7, GraphPad) with a one site Ki equation constrained for the value of Kd of the obtained for ARS-boronate and for the concentration of the ARS (100 μM) to obtain the Ki value of the boronate for the chosen carbohydrate.

TABLE 1
Glucose and lactate Kd-values as determined by the alizarin
assay described in Example A for diboron compounds used
in the compounds of the invention and for monoboron
compounds included as comparison.

| | Binding Affinity | Binding Affinity |
|---|---|---|
| Diboron/monoboron | Kd Glucose | Kd Lactate |
| of Example No. | (mM) | (mM) |
| Example 10 | 2.6 | 67.9 |
| Example 18 | 3.2 | 352.0 |
| Example 19 | 1.6 | 107.0 |
| Example 23 | 1.5 | 24.1 |
| Example 24 | 1.1 | 41.0 |
| Example 28 | 1.3 | 11.0 |
| Example 29 | 0.52 | N.D. |
| Example 32 | 0.80 | 40.0 |
| Example 35 | 4.2 | 13.0 |
| Example 36 | 3.2 | 60.6 |
| Example 38 | 0.8 | 100.0 |
| Example 41 | | |
| (comparison compound) | 40.0 | 66.0 |
| Example 42 | | |
| (comparison compound) | 10.0 | N.D. |
| Example 43 | | |
| (comparison compound) | 50.0 | 200.0 |

N.D. = not detectable

Data in table 1 show that the diboron compounds used in the compounds of the invention bind glucose with Kd values in the low millimolar range (0.8 to 4.2 mM), and that the given diboron compounds have higher affinity towards glucose than towards lactate. Data in table 1 also show that monoborons (Example 41, 42, 43) have weaker affinity to glucose than the diboron compounds used in the compounds of the invention. Monoborons do not respond well to fluctuations in physiological range for glucose concentrations.

### Example B: Assay to Determine Affinity to the Human Insulin Receptor (hIR-A) in Absence or Presence of Glucose

Insulin Receptor Preparation

BHK cells over-expressing human Insulin Receptor A (hIR-A) were lysed in 50 mM Hepes pH 8.0, 150 mM NaCl, 1% Triton X-100, 2 mM EDTA and 10% glycerol. The cleared cell lysate was batch absorbed with wheat germ agglutinin (WGA)-agarose (Lectin from Triticum vulgaris-Agarose, L1394, Sigma-Aldrich Steinheim, Germany) for 90 minutes. The receptors were washed with 20 volumes 50 mM Hepes pH 8.0, 150 mM NaCl and 0.1% Triton X-100, where after the receptors were eluted with 50 mM Hepes pH 8.0, 150 mM NaCl, 0.1% Triton X-100, 0.5 M n-Acetyl Glucosamine and 10% glycerol. All buffers contained Complete (Roche Diagnostic GmbH, Mannheim, Germany) as described in Andersen et al. 2017 PLos One 12.

Insulin Receptor Scintillation Proximity Assay SPA Binding Assay

SPA PVT anti-mouse beads (Perkin Elmer) were diluted in SPA binding buffer, consisting of 100 mM Hepes, pH 7.4 or pH 7.8, 100 mM NaCl, 10 mM MgSO₄, 0.025% (v/v) Tween-20. SPA beads were incubated with the IR-specific antibody 83-7 (Soos et al. 1986 Biochem J. 235, 199-208) and solubilized semi-purified HIR-A. Receptor concentrations were adjusted to achieve 10% binding of 5000 cpm ¹²⁵I-(Tyr31)-Insulin (Novo Nordisk A/S). Dilution series of cold ligands were added to 96-well Optiplate, followed by tracer (¹²⁵I-Insulin, 5000 cpm/well) and lastly receptor/SPA mix. In order to test the glucose sensitivity, the binding experiments were set up in absence or presence of 20 mM glucose. The plates were rocked gently for 22.5 hours at 22° C., centrifuged for 5 minutes at 1000 rpm and counted in TopCounter (Perkin Elmer). Data points were fitted to a four-parameter logistic model, whereby the relative affinity of the analogue compared to human insulin (within the same plate) was determined. The relative affinities for the analogues compared to human insulin were determined as fold change and the increase in relative affinity from 0 to 20 mM glucose (HIR glucose factor) reflected the glucose sensitivity of the analogues. The experiments were done in presence of 1.5% HSA. Data is shown in table 2.

TABLE 2
Insulin receptor affinity in the presence of 1.5% HSA, glucose
factor and LCMS data for compounds of the invention

| | HIR | | | | | |
|---|---|---|---|---|---|---|
| | affinity | HIR | | | | |
| | relative | glucose | | | | |
| | HI | factor, | | | | |
| | (%), | 20 mM | | | | |
| Com- | 1.5% | glucose | | | | |
| pound | HSA | 1.5% | | | LCMS | LCMS |
| of | (pH 7.8 | HSA (pH | LCMS | LCMS | [M + | [M + |
| Exam- | or 7.4, | 7.8 or | [M + | [M + | 4H]⁴⁺ | 5H]⁵⁺ |
| ple | see | 7.4, see | 4H]⁴⁺ | 5H]⁵⁺ | cal- | cal- |
| No | note) | note) | found | found | culated | culated |
| 101 | 2.9 | 2.7 | 1670.4 | | 1670.6 | |
| 102 | 6.0 | 3.2 | 1689.0 | | 1689.2 | |
| 103 | 2.1 | 0.5 | 1663.0 | | 1664.1 | |
| 104 | 0.7 | 0.4 | 1686.0 | 1346.0 | | |
| 105 | 2.9 | 3.2 | 1827.51 | 1458.5 | 1827.3 | 1462.0 |
| 106 | 0.6 | 1.7 | 1724.3 | | 1724.2 | |
| 107 | 0.9 | 4.2 | 2055.3 | 1640.8 | 2055.3 | 1640.9 |
| 108 | 0.6 | 3.1 | 1987.5 | | 1987.5 | |
| 109 | 6.4 | 1.5 | 2026.3 | 1603.0 | 2026.1 | 1603.0 |
| 110 | 16.1 | 1.3 | 1643.7 | | 1643.7 | |
| 111 | 0.6 | — | | | | |
| 112 | 3.8 | 7.6 | 2198.6 | 1759.1 | 2202.0 | 1762.0 |
| 113 | 2.4 | 2.2 | | | | |
| 114 | 6.8 | 4.5 | 1639.6 | 1318.9 | 1639.3 | 1326.3 |
| 115 | 1.4 | 9.1 | 2100.9 | 1680.9 | 2100.9 | 1680.9 |
| 116 | 3.8 | 5.7 | 2069.9 | 1656.1 | 2069.9 | 1656.1 |
| 117 | 9.2 | — | | | | |
| 118 | 4.0 | 4.5 | | | | |
| 119 | 1.5 | 9.0 | | 1722.7 | | 1722.4 |
| 120 | 1.7 | 2.0 | | | | |
| 121 | 14.6 | — | | 1733.1 | | 1732.8 |
| 122 | 1.9 | 3.9 | 1994.8 | 1596.8 | 1994.3 | 1595.7 |
| 123 | 12.0 | 1.3 | 2090.9 | 1673.0 | 2090.9 | 1672.9 |
| 124 | 2.1 | 3.4 | 1684.0 | 1348.0 | | |
| 125 | 6.2 | 2.0 | 1992.0 | 1587.0 | | |
| 126 | 9.7 | 2.3 | 1686.0 | 1342.0 | | |
| 127 | 1.5 | 3.5 | 1606.1 | | 1605.9 | |
| 128 | 25.6 | 1.9 | 1673.9 | 1339.5 | 1678.6 | 1343.1 |
| 129 | .6 | 4.3 | 1601.3 | | 1601.6 | |
| 130 | 11.9 | 2.1 | 1656.7 | 1325.6 | 1656.7 | 1325.6 |
| 131 | .5 | 3.0 | 1694.8 | 1352.4 | 1694.8 | 1352.4 |
| 132 | 3.7 | 4.0 | | 1726.3 | | 1726.2 |
| 133 | 5.7 | 2.8 | 1883.6 | | 1883.3 | |
| 134 | 8.0 | — | 1595.9 | | 1596.1 | |
| 135 | 1.2 | 20.9 | | 1650.6 | | 1650.4 |
| 136 | 3.2 | 3.4 | | | 1847.7 | |
| 137 | 2.9 | 13.7 | 1590.0 | 1268.0 | | 1590.0 |
| 138 | .9 | 9.9 | 1506.9 | | 1507.0 | 1506.9 |
| 139 | 4.0 | 3.2 | 1603.3 | | 1603.1 | 1603.3 |
| 140 | 2.0 | 13.8 | 1789.0 | | 1788.6 | 1789.0 |
| 141 | 8.6 | 2.3 | 1634.3 | | 1634.1 | |
| 142 | 1.8 | 2.2 | 1879.1 | | 1878.6 | |
| 143 | 4.8 | 2.9 | 1596.0 | | 1596.1 | 1596.0 |
| 144 | 2.9 | 2.3 | 1633.3 | | 1633.2 | 1633.3 |
| 145 | 16.9 | 1.5 | 1633.2 | | 1633.3 | 1633.2 |
| 146 | 3.5 | 4.5 | 1544.3 | | 1544.0 | 1544.3 |
| 147 | 13.1 | 5.1 | 1674.0 | 1336.0 | | |
| 148 | 8.8 | 1.8 | 1666.0 | 1329.0 | 1670.0 | 1336.0 |
| 149 | 6.7 | 2.8 | 1638.8 | | 1638.6 | |
| 150 | 13.3 | 1.3 | 1647.4 | | 1647.0 | |
| 151 | 4.6 | 3.0 | 1622.0 | | 1622.1 | |
| 152 | 4.9 | 2.9 | 1653.1 | | 1653.0 | |
| 153 | 12.4 | 1.9 | 1647.6 | | 1647.3 | |
| 154 | 6.1 | 3.7 | 1640.6 | | 1640.4 | |
| 155 | 5.3 | 3.0 | 1626.3 | | 1626.1 | |
| 156 | 1.4 | 1.7 | 1804.5 | 1440.2 | 1804.5 | 1440.2 |
| 157 | 7.2 | 8.0 | 1667.0 | 1334.0 | | 1667.0 |
| 158 | 2.0 | 9.9 | 1558.5 | | 1558.3 | 1558.5 |
| 159 | 5.2 | 15.2 | 1603.0 | 1275.0 | | 1603.0 |
| 160 | 3.0 | 15.1 | | 1657.8 | | 1657.6 |
| 161 | 1.3 | 7.7 | 1604.9 | | | 1604.7 |
| 162 | 5.0 | 2.0 | 1756.6 | 1401.9 | 1756.7 | 1401.9 |
| 163 | 1.8 | 7.6 | 1666.5 | 1329.8 | 1666.7 | 1329.9 |
| 164 | 0.4 | 3.3 | 1873.2 | 1491.4 | 1873.2 | 1491.4 |
| 165 | 0.2 | 14.4 | 1729.2 | 1376.2 | 1729.0 | 1376.2 |
| 166 | 2.2 | 5.9 | 1553.3 | | 1553.1 | |
| 167 | 1.4 | 14.2 | 1798.0 | | 1797.6 | |
| 168 | 2.8 | 2.3 | 2092.8 | 1670.9 | 2093.1 | 1671.0 |
| 169 | 1.7 | 9.7 | 1998.2 | 1595.2 | 1998.5 | 1595.4 |
| 170 | 5.3 | 1.9 | 1666.5 | 1329.8 | 1666.7 | 1329.9 |
| 171 | 0.9 | 5.7 | 1873.2 | 1491.4 | 1873.2 | 1491.4 |
| 172 | 3.2 | 2.2 | 1729.2 | 1376.2 | 1729.0 | 1376.2 |
| 173 | 0.7 | 21.2 | 1731.9 | 1381.9 | 1731.9 | 1381.9 |
| 174 | 0.5 | 14.1 | 1627.6 | 1298.5 | 1627.6 | 1298.7 |
| 175 | 0.1 | 9.3 | 1913.1 | 1523.5 | 1913.2 | 1523.6 |
| 176 | 0.4 | 46.2 | 1477.1 | | 1477.4 | |
| 177 | 0.4 | 9.0 | 1566 | 1250 | | |
| 178 | 0.5 | 7.2 | 1548.0 | 1239.0 | | |
| 179 | 1.4 | 8.4 | 1661 | 1325 | | |
| 180 | 0.3 | 8.0 | 1559.0 | 1247.0 | | |
| 181 | 0.7 | 15.2 | 1794.5 | 1435.8 | 1794.2 | 1435.7 |
| 182 | 0.2 | 15.3 | 1540 | | 1540.3 | |
| | | | 1540 | | | |
| 183 | 0.37 | 11.14 | 1720.1 | | 1720.3 | |
| 184 | 1.05 | 10.53 | 1550.5 | | 1550.5 | |
| 185 | 1.11 | 27.18 | 1749.9 | 1396.4 | 1749.8 | 1396.5 |
| 186 | 0.01 | 2.5 | 1990.8 | 1593.1 | 1991.5 | 1593.4 |
| 187 | 1.58 | 11.88 | 1709.3 | 1363.9 | 1709.2 | 1363.9 |
| 188 | 0.21 | 8.23 | 1877.5 | | 1877.3 | |
| 189 | 0.23 | 4.25 | 1504.6 | | 1504.5 | |
| 190 | 0.1 | 4.6 | 1772.2 | 1414.2 | 1772.4 | 1414.5 |
| 191 | 0.03 | 2.9 | 1896.3 | 1513.6 | 1894.5 | 1513.8 |
| 192 | 0.03 | 1.3 | 1787.5 | 1426.6 | 1787.5 | 1426.6 |
| 193 | 2.60 | 7.34 | 1871.1 | | 1871.2 | |
| 194 | 15.36 | 5.83 | 1520.0 | 1216.0 | | |
| 195 | 1.17 | 5.68 | 1367.0 | | | |
| 196 | 1.19 | 6.35 | 1717.0 | 1370.0 | | |
| 197 | 0.03 | 8.3 | 1800.0 | 1433.0 | | |
| 198 | 0.2 | 1.9 | 1907.8 | 1526.5 | 1908.1 | 1526.7 |
| 199 | 0.02 | 2.3 | 2100.0 | 1676.6 | 2099.9 | 1676.5 |
| 200 | 1.0 | 3.6 | 1464.5 | | 1464.9 | |
| 201 | 0.1 | 1.9 | 1543.0 | | 1543.3 | |
| 202 | 0.04 | 2.3 | 1939.0 | 1548.0 | | |
| 203 | 4.6 | 1.6 | 1919.4 | | 1919.6 | |
| 204 | 0.6 | 7.6 | 1969.4 | | 1969.6 | |
| 205 | 0.1 | 9.7 | 1607.5 | 1286.3 | 1607.6 | 1286.2 |
| 206 | 0.3 | 5.7 | 1699.0 | 1348.6 | 1699.1 | 1348.7 |
| 207 | 2.6 | 3.1 | 1823.6 | 1458.8 | 1823.6 | 1459.0 |
| 208 | 1.39 | 13.09 | 1926.3 | 1541.2 | 1926.4 | 1539.3 |
| 209 | 0.5 | 1.95 | 2021.9 | 1617.8 | 2021.9 | 1617.7 |
| 210 | 0.7 | 6.3 | 1962.3 | | 1962.5 | |
| 211 | 0.1 | 8.1 | 1649.7 | 1319.9 | 1649.7 | 1320.0 |
| 212 | 0.12 | 2.65 | 1608.9 | | | 1609.2 |
| 213 | 0.1 | 12.6 | 1586.6 | 1265.9 | 1586.7 | 1265.9 |
| 214 | 0.2 | 12.3 | | 1890.46 | | 1890.58 |
| 215 | 0.5 | 16.2 | 1614.6 | | 1614.5 | |
| 216 | 0.1 | 6.2 | 1781.8 | | 1781.6 | |
| 217 | 0.4 | 4.9 | 1862.0 | | 1862.1 | |
| 218 | 1.3 | 7.3 | 1980.5 | | 1980.4 | |
| 219 | 0.2 | 5.5 | 1753.2 | | 1753.2 | |
| 220 | 1.6 | 10.9 | 1840.3 | | 1840.6 | |
| 221 | 3.8 | 11.4 | 1959.0 | | 1958.9 | |
| 222 | 0.9 | 9.2 | 1731.7 | | 1731.5 | |
| 223 | 67.3 | 2.8 | 1579.0 | 1263.0 | | |
| 224 | 40.9 | 1.8 | 1579.0 | 1263.0 | | |
| 225 | 2.9 | 14.2 | 1698.0 | 1359.0 | | |
| 226 | 0.5 | 3.6 | 1822.0 | 1458.0 | | |
| 227 | 0.3 | 14.6 | 1752.0 | 1399.0 | | |
| 228 | 0.03 | 3.1 | 1897.0 | 1518.0 | | |
| 229 | 2.0 | 5.3 | 1720.0 | 1376.0 | | |
| 230 | 0.9 | 11.7 | 1586.2 | | 1586.0 | |
| 231 | 2.8 | 4.5 | 1947.9 | | 1947.9 | |
| 232 | 0.5 | 18.1 | 1524.2 | 1215.9 | 1524.2 | 1215.9 |
| 233 | 0.3 | 12.1 | 1997.9 | | 1997.9 | |
| 234 | 0.1 | 8.8 | 1636.0 | | 1636.5 | |
| 235 | 0.03 | 3.6 | 1649.2 | | 1649.1 | |
| 236 | 16.6 | 1.4 | 1599.1 | | 1599.1 | |
| 237 | 3.8 | 1.8 | 1960.9 | | 1960.9 | |
| 238 | 4.1 | 2.6 | 1919.6 | | 1919.6 | |
| 239 | 1.1 | 10.2 | 1906.6 | | 1906.6 | |
| 240 | 0.2 | 7.5 | 1956.7 | | 1956.6 | |
| 241 | 0.3 | 4.9 | 1928.1 | | 1928.2 | |
| 242 | 0.1 | 1.5 | 1969.7 | | 1969.6 | |
| 243 | 0.8 | 5.1 | 1758.2 | | 1758.1 | |
| 244 | 0.2 | 40.5 | 1929.2 | | 1929.4 | |
| 245 | 16.4 | 6.2 | 1603 | 1283 | | |
| 246 | 9.1 | 3.2 | 1604 | 1283 | | |
| 247 | 13.3 | 9.0 | 1589.0 | 1272.0 | | |
| 248 | 14.7 | 8.2 | 1589.0 | 1272.0 | | |
| 249 | 1.2 | 2.5 | 1854.0 | 1480.0 | | |
| 250 | 4.1 | 4.9 | 1562.0 | 1250.0 | | |
| 251 | 0.02 | 3.5 | 1895.0 | 1513.0 | | |
| 252 | 0.1 | 3.6 | 1771 | 1413 | | |
| 253 | 5.4 | 7.2 | 1560.0 | 1245.0 | | |
| 254 | 0.1 | 18.6 | 1918.1 | | 1918.3 | |
| 255 | 0.04 | 26.1 | 1551.7 | | 1552.0 | |
| 256 | 0.1 | 6.2 | 1872.5 | | 1872.5 | |
| 257 | 14.4 | 5.4 | 1586.0 | 1269.0 | | |
| 258 | 8.9 | 2.0 | 1586.0 | 1269.0 | | |
| 259 | 4.2 | 11.0 | 1562.0 | 1250.0 | | |
| 260 | 0.2 | 10.1 | 1673.0 | 1335.0 | | |
| 261 | 0.1 | 2.3 | 1784.0 | 1420.0 | | |
| 262 | 0.6 | 27.7 | 1888.5 | | 1888.9 | |
| 263 | 0.3 | 13.7 | 1770.4 | | 1770.5 | |
| 264 | 0.2 | 5.2 | 1683.0 | 1347.0 | | |
| 265 | 0.01 | 5.9 | 1818.0 | 1451.0 | | |
| 266 | 0.1 | 6.1 | 1673.7 | | 1673.7 | |
| 267 | 0.1 | 8.0 | 1632.0 | 1306.0 | | |
| 268 | 0.4 | 7.5 | 1831.9 | 1465.4 | 1832.1 | 1465.9 |
| 269 | 0.01 | 19.0 | 1743.0 | 1391.0 | | |
| 270 | 0.1 | 1.8 | 1743.0 | 1391.0 | | |
| 271 | 0.4 | 7.1 | 1620.0 | 1296.0 | | |
| 272 | 0.2 | 12.1 | 1712.0 | 1370.0 | 1710.9 | |
| 273 | 0.1 | 20.4 | 1537.6 | | 1537.7 | |
| 274 | 0.2 | 3.9 | 1593.1 | | 1593.0 | |
| 275 | 0.1 | 6.9 | 1621.6 | | 1621.8 | |
| 276 | 0.7 | 10.1 | 1571.7 | | 1571.8 | |
| 277 | 0.2 | 32.0 | 1943.5 | | 1943.6 | |
| 278 | 0.02 | 26.8 | 1594.1 | 1271.9 | 1594.2 | 1271.9 |
| 279 | 0.1 | 7.2 | 1553.6 | 1239.5 | 1553.7 | 1239.5 |
| 280 | 0.5 | 18.0 | 2027.5 | 1625.8 | 2027.6 | 1625.9 |
| 281 | 5.1 | 7.6 | 1972.8 | 1585.9 | 1973.1 | |
| 282 | 1.4 | 7.1 | 2003.6 | 1336.1 | | |
| 283 | 1.4 | 5.3 | 1623.7 | 1299.2 | 1623.7 | 1299.2 |
| 284 | 0.5 | 55.0 | 1902.8 | | 1903.1 | |
| 285 | 0.1 | 19.7 | 1501.7 | | 1501.7 | |
| 286 | 1.1 | 8.4 | 1915.6 | | 1915.8 | |
| 287 | 1.2 | 7.0 | 1836.7 | | 1837.0 | |
| 288 | 0.5 | 22.3 | 1824.2 | | 1824.3 | |
| 289 | 0.3 | 24.9 | 1745.2 | | 1745.5 | |
| 290 | 0.5 | 8.0 | 1623.5 | 1299.1 | 1623.6 | 1299.1 |
| 291 | 0.1 | 11.9 | 1785.9 | | 1786.0 | |
| 292 | 0.1 | 16.3 | 1864.7 | | 1864.8 | |
| 293 | 0.2 | 7.7 | | 1623.5 | | 1623.6 |
| 294 | 0.3 | 7.0 | | 1729.5 | | 1729.5 |
| 295 | 1.4 | 1.8 | 1893.8 | 1512.1 | 1894.1 | 1511.9 |
| 296 | 0.2 | 8.5 | | 1662.4 | | 1662.2 |
| 297 | 0.3 | 4.4 | 1860.3 | 1488.6 | 1860.6 | 1488.7 |
| 298 | 3.4 | 9.0 | 1815.4 | | 1815.5 | |
| 299 | 2.3 | 14.3 | 1865.4 | | 1865.5 | |
| 300 | 0.3 | 14.5 | 1944.2 | | 1944.4 | |
| 301 | 0.3 | 18.4 | 1458.7 | | 1458.7 | |
| 302 | 0.3 | 15.0 | 1860.2 | | 1860.1 | |
| 303 | 1.8 | 16.2 | 1815.4 | | 1815.5 | |
| 304 | 1.1 | 15.0 | 1781.3 | | 1781.3 | |
| 305 | 1.0 | 13.5 | 1702.1 | | 1702.5 | |
| 306 | 2.3 | 4.6 | 1994.6 | | 1994.8 | |
| 307 | 1.0 | 14.8 | | | | |
| 308 | 1.8 | 15.1 | 1549.3 | | 1549.0 | |
| 309 | 0.3 | 6.2 | 1688.1 | | 1688.1 | |
| 310 | 0.1 | 11.1 | 1716.6 | | 1716.6 | |
| 311 | 0.2 | 8.1 | 1625.0 | | 1625.1 | |
| 312 | 0.04 | 7.8 | 1637.2 | | 1637.1 | |
| 313 | 0.5 | 4.0 | 1593.1 | | 1593.3 | |
| 314 | 1.8 | 4.3 | 1915.9 | | 1915.8 | |
| 315 | 1.2 | 5.3 | 1837.1 | | 1837.0 | |
| 316 | 0.5 | 8.2 | 1714.6 | | 1714.6 | |
| 317 | 0.5 | 2.7 | 1649.8 | 1319.9 | 1649.7 | 1320.0 |
| 318 | 1.3 | 2.9 | 1969.4 | | 1969.4 | |
| 319 | 1.1 | 5.7 | 1935.8 | | 1935.8 | |
| 320 | 0.4 | 5.0 | 1559.5 | | 1559.7 | |
| 321 | 0.6 | 5.4 | | 1957.9 | | 1957.7 |
| 322 | 1.8 | 9.5 | 1882.2 | | 1882.3 | |
| 323 | 0.2 | 46.9 | 1680.9 | 1341.4 | 1680.9 | 1341.3 |
| 324 | 0.3 | 46.5 | 2056.4 | 1645.3 | 2056.7 | 1645.6 |
| 325 | 2.8 | 8.9 | 1977.0 | 1581.0 | | |
| 326 | 1.3 | 10.1 | 1803.5 | | 1803.5 | |
| 327 | 0.1 | 32.2 | 1745.7 | | 1745.7 | |
| 328 | 2.1 | 3.5 | 1611.2 | 1289.1 | 1611.2 | 1289.1 |
| 329 | 5.5 | 18.0 | 1650.5 | | 1651.2 | |
| 330 | 1.1 | 4.1 | 2034.3 | 2542.5 | 2542.9 | 2034.5 |
| 331 | 0.3 | 43.6 | 1973.7 | | 1973.4 | |
| 332 | 0.3 | 33.9 | | 1622.0 | | 1622.0 |
| 333 | 0.2 | 42.9 | 1894.7 | | 1894.5 | |
| 334 | 0.04 | 17.2 | 1707.2 | 1366.0 | 1707.2 | 1366.0 |
| 335 | 0.1 | 29.8 | 1663.2 | 1330.8 | 1663.2 | 1330.8 |
| 336 | 0.2 | 19.5 | 1801.0 | 1441.3 | 1801.0 | 1441.0 |
| 337 | 0.7 | 7.0 | 1895.0 | | 1894.6 | |
| 338 | 0.1 | 7.4 | | | | |
| 339 | 0.1 | 20.8 | 1986.0 | 1588.0 | | |
| 340 | 6.4 | 3.7 | 1681.9 | | 1681.2 | |
| 341 | 0.4 | 7.7 | 1575.4 | | 1575.3 | |
| 342 | 2.8 | 8.7 | 1898.3 | | 1897.8 | |
| 343 | 2.4 | 9.0 | 1819.2 | | 1819.0 | |
| 344 | 2.1 | 3.8 | 1951.8 | | 1951.4 | |
| 345 | 0.04 | 19.7 | | 1602.8 | | 1602.3 |
| 346 | 0.5 | 5.3 | 1627.0 | | 1627.2 | |
| 347 | 0.3 | 73.5 | 2013.0 | 1603.0 | | |
| 348 | 0.2 | 7.3 | 1701.0 | 1358.0 | | |
| 349 | 1.0 | 4.7 | 1665.0 | 1332.0 | | |
| 350 | 0.1 | 18.4 | 1741.0 | 1393.0 | | |
| 351 | 0.2 | 2.4 | 2064.0 | 1651.0 | | |
| 352 | 0.4 | 5.2 | 1748.0 | 1398.0 | | |
| 353 | 1.3 | 5.2 | 1909.8 | 1528.2 | 1909.3 | 1527.6 |
| 354 | 0.1 | 3.2 | 1668.0 | | 1666.0 | |

The data in table 1 show that the diboron insulin conjugates of the invention in presence of 1.5% HSA have higher insulin receptor affinity in presence of 20 mM glucose than when no glucose is present. Glucose can displace the diboron insulin conjugates from binding to albumin, thereby giving a higher free fraction of non-albumin bound diboron insulin conjugate, resulting in netto higher insulin receptor affinity.

### Example C: Assay to Determine Glucose-Sensitive Signalling (AKT Phosphorylation in Low/High Glucose), Table 3

When insulin binds to the Insulin Receptor (IR) it induces activation of downstream signaling pathways. One of the downstream signaling molecules is AKT, and AKT phosphorylation can thus be used to monitor the activation of the insulin signaling pathway.

AKT Assay

Chinese Hamster Ovary cells overexpressing the HIR-A were cultivated at 37° C., and plated in 96-well plates with either 3 mM or 20 mM glucose concentration. Increasing amounts of human insulin or insulin derivatives of the invention to generate concentration-response curves were added and incubated for 10 min. The media was discarded and the cells place on ice. The AKT activation assay was done as described by the vendor using AlphaScreen® SureFire®. The signals were measured with Envision instrument (EnVision, Perkin Elmer). The fold change between the potency of the glucose sensitive analogue (relative to human insulin) at 20 mM and 3 mM glucose concentration was determined.

### Example D: Assay to Determine Carbohydrate-Sensitive Glucose Uptake in Cells (Rat Lipogenesis Assay)

When insulin binds to the insulin receptor it induces activation of downstream signaling pathways. One metabolic endpoint of insulin signaling is lipid metabolism, and the lipogenesis assay was used to measure an end point read-out because in presence of insulin, ³H-glucose uptake by the cells is stimulated and is incorporated into lipids.

Rat Lipogenesis Assay (rFFC)

Epidydimal fat pads from Sprague Dawley rat were degraded with collagenase in Hepes Krebs Ringer Buffer at 36.5° C. for 1-1.5 hours under vigorous shaking. The suspension was filtered through 2 layers of gauze. The phases were separated by 5 min standing at room temperature, allowing the adipocytes to collect in the upper phase. The lower phase was removed with a syringe. The adipocytes were washed twice with 20 ml Hepes Krebs Ringer Buffer. Cells were transferred to 96 well plates in Hepes Krebs Ringer buffer containing 1.5% HSA, 0.5 mM glucose, 0.1 μCi/well glucose (D-[3-³H] glucose (20.0 Ci/mmol) Perkin Elmer), +/−10 mM sorbitol. Increasing amounts of human insulin or insulin derivatives of the invention to generate concentration-response curves were added and incubated for 2 hours at 36.5° C.

The reactions were stopped by addition of 100 μL Microscient E (cat #6013661 Perkin Elmer). The plates rested 3 hours before counting in Top counter. The ratio between EC50 no sorbitol/EC50 10 mM sorbitol of the glucose sensitive analogous was determined.

TABLE 3

| | AKT 1.5% HSA | rFFC 1.5% HSA |
|---|---|---|
| Compound of | 3 vs 20 mM | 0 vs 10 mM |
| Example No. | glucose factor | sorbitol factor |
| 179 | 2.3 | 10.5 |
| 181 | 3.2 | 2.8 |
| 204 | 2.0 | 5.9 |
| 205 | 1.9 | 4.0 |
| 210 | 2.6 | 5.4 |
| 211 | 2.2 | 5.4 |
| 214 | 5.7 | — |
| 215 | 2.3 | 18.0 |
| 216 | 2.3 | 4.4 |
| 217 | 2.6 | 12.8 |
| 218 | 3.0 | 16.3 |
| 219 | 2.3 | — |
| 222 | 1.6 | 10.1 |
| 225 | 2.6 | 17.9 |
| 227 | 2.4 | 16.7 |
| 229 | 2.0 | 11.5 |
| 230 | 2.4 | 9.5 |
| 233 | 2.4 | 12.0 |
| 234 | 1.8 | 5.4 |
| 239 | 1.7 | 15.0 |
| 240 | 2.4 | 9.4 |
| 241 | — | 11.7 |
| 244 | 6.4 | 28.1 |
| 264 | 1.7 | 10.4 |
| 272 | 3.3 | 21.1 |
| 273 | 3.5 | 9.2 |
| 274 | 2.3 | 13.1 |
| 275 | 1.9 | 6.9 |
| 276 | 2.3 | 9.9 |
| 280 | 4.1 | 10.1 |
| 285 | 2.4 | 11.0 |
| 286 | 2.3 | 11.3 |
| 287 | 2.2 | 10.2 |
| 288 | 3.3 | 9.4 |
| 289 | 3.0 | 19.1 |
| 291 | 3.1 | — |
| 292 | 3.8 | 17.1 |
| 294 | 1.7 | 6.5 |
| 300 | 2.9 | 14.7 |
| 301 | 3.3 | 21.9 |
| 309 | 1.7 | 11.6 |
| 310 | 2.6 | 7.3 |
| 316 | 1.8 | 5.9 |
| 326 | 2.2 | 27.6 |
| 331 | — | 16.1 |
| 332 | — | 13.4 |
| 333 | — | 13.5 |
| 335 | — | 7.4 |

The AKT data in table 3 show that the diboron insulin conjugates of the invention give higher levels of AKT phosphorylation in presence of higher glucose concentrations (20 mM) versus lower glucose concentrations (3 mM). The lipogenesis data in table 3 show that the diboron insulin conjugates of the invention give higher levels of lipogenesis (ie more glucose transport) in the presence of higher levels of sugar (10 mM sorbitol) compared to no added sugar (0 mM sorbitol).

The cells need glucose to survive, so 3 mM glucose was used as lower level, and 20 mM as higher level. The rFFC assay is itself sensitive to glucose levels, so sorbitol (which don't affect glucose transport in itself) was used as sugar to displace diboron-insulin derivatives from HSA in the rFFC assay.

### Example E: PK and PD Data

Euglycaemic and hyperglycaemic clamp were performed in 65-100 kg naïve female domestic pigs. The animals were instrumented with two venous catheters one for infusion and one for sampling of blood. Basal replacement was performed by constant infusion of somatostatin, glucagon and human insulin. After infusion start the plasma glucose level was changed to 10 mM or 3.5-4 mM by adjusting the g glucose infusion. After plasma glucose steady state (90 or 120 min) a i.v. bolus of an insulin analog was delivered. For pharmacokinetic (PK) analysis plasma was sampled at selected timepoints for 360 to 510 min and analyzed specifically for the analog. For pharmadynamic (PD) analysis the change in glucose infusion rate from steady state was used.

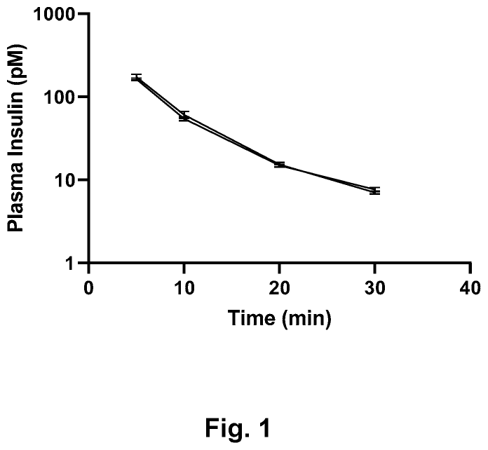

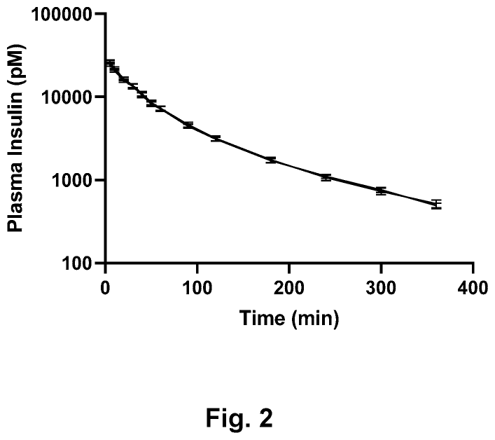

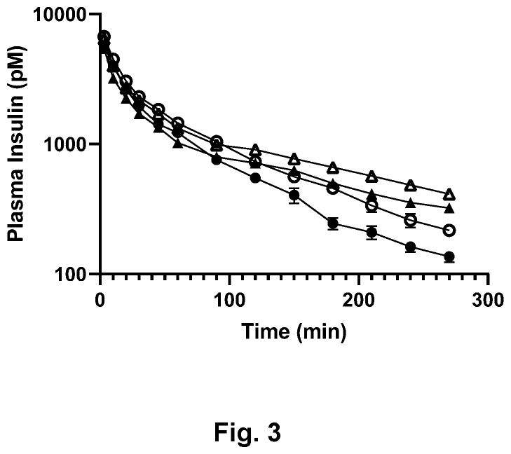

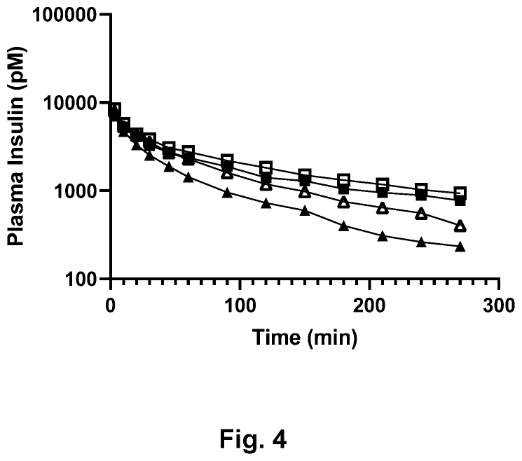

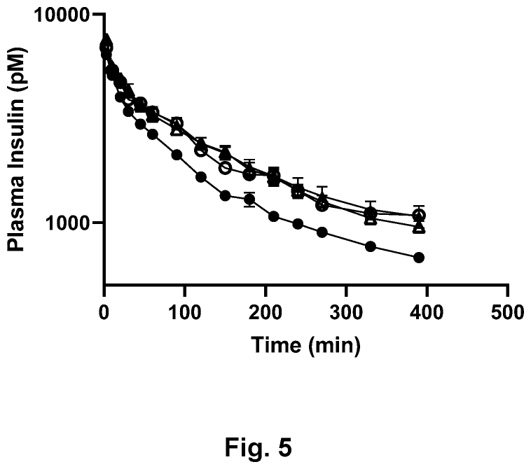

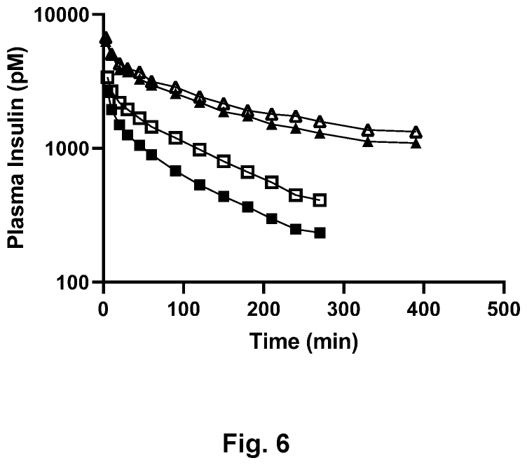

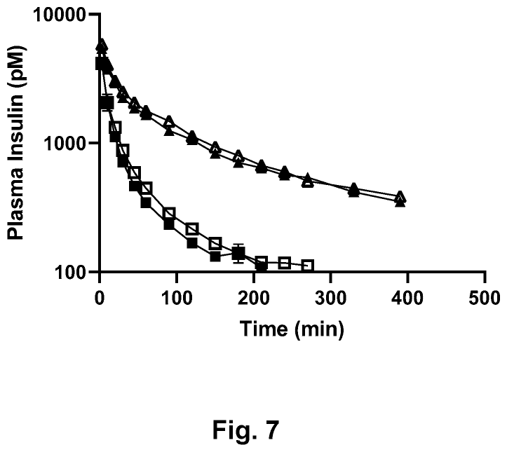

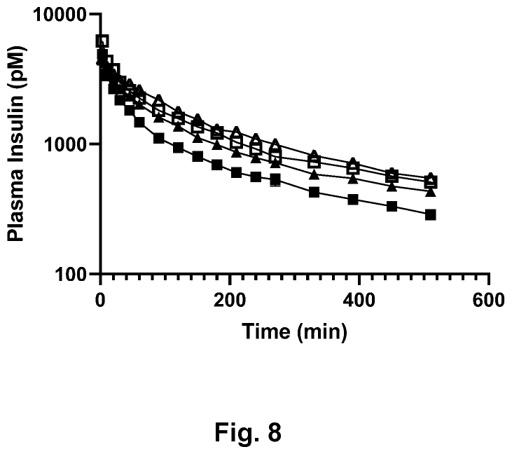

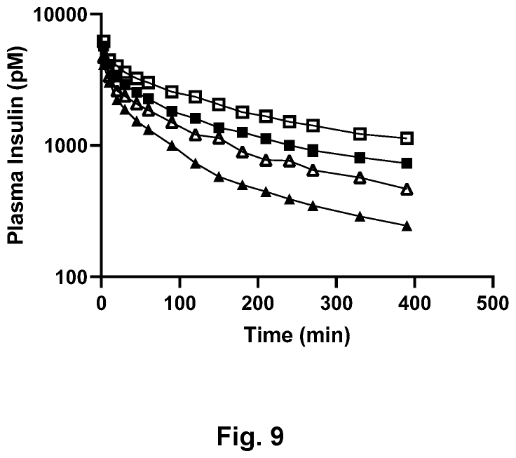

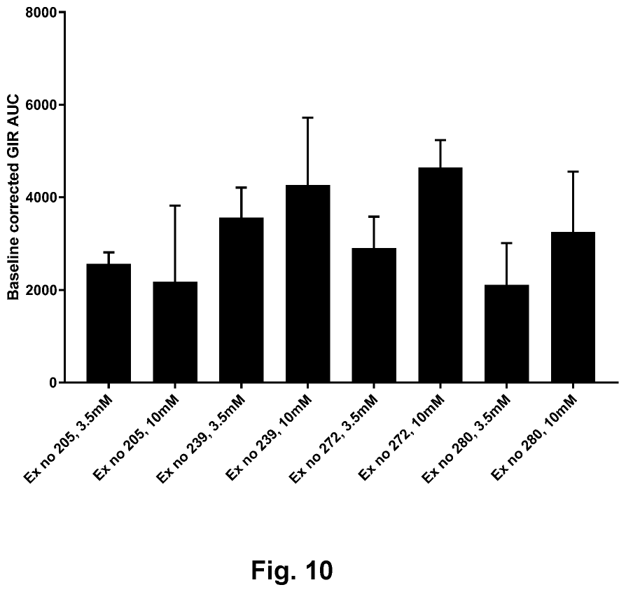

Glucose-sensitive PK data for insulin derivatives of the invention, as well as controls, by i.v. dosing to pigs clamped at 3.5-4 or 10 mM glucose are shown in FIGS. 1-9, and PD data as baseline-adjusted glucose infusion rate areas under the curves, for clamps at 3.5-4 mM glucose vs 10 mM glucose is shown in FIG. 10.

The pig PK data show that diboron insulin conjugates of the invention are cleared faster at higher blood glucose levels (10 mM) compared to lower glucose levels (3.5-4 mM). The displacement of diboron insulin conjugates from albumin binding by glucose give raise to larger fraction of unbound insulin, thus available for insulin receptor binding and activation. The pig PD data show diboron insulin conjugates of the invention give raise to more glucose disposal at high glucose blood glucose levels compared to low glucose level. Contrary, non-glucose-sensitive insulin controls (insulin aspart and insulin degludec) show the same PK and PD in pigs clamped at high and low blood glucose levels.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound comprising
i) human insulin or a human insulin analogue; and
ii) two or more modifying groups M, wherein each of the modifying groups M comprises two aryl moieties, wherein a boron atom is attached to each of the two aryl moieties; and
wherein each of the two or more modifying groups M is attached, optionally via a spacer, to the amino group of the N-terminal amino acid residue of the A-chain or B-chain of said human insulin or human insulin analogue or to the epsilon amino group of a lysine in said human insulin or human insulin analogue; and
wherein each of the modifying groups M is independently selected from the group of 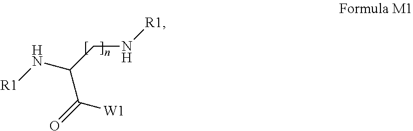
which represents a D- or an L-amino acid form, and
wherein n represents an integer in the range of 1 to 4;
wherein W1 is absent and represents the point of attachment * to said human insulin or human insulin analogue, or W1 represents
NH—CH₂—C(═O)—*,
NH—CH₂CH₂—C(═O)—*,
the D- or L-form of NH—CH(COOH)—CH₂CH₂—C(═O)—*,
the D- or L-form of NH—CH(COOH)—CH₂CH₂—C(═O)—NH—CH₂CH₂—C(═O)—*, or
NH—CH₂CH₂—C(═O)—NH—(CH₂)₂—O—(CH₂)₂—O—CH₂—CO—*,
wherein * represents the point of attachment to said human insulin or human insulin analogue; and
wherein R1 is selected from 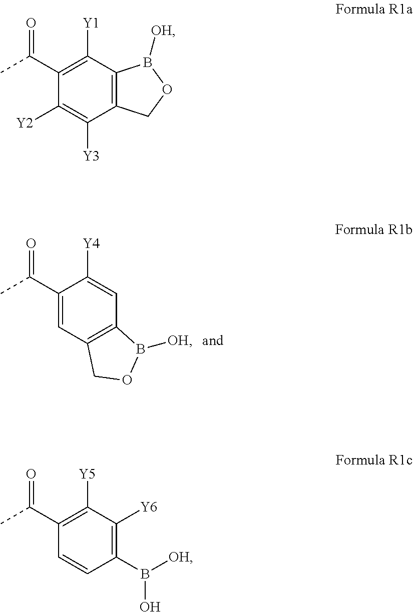
wherein Y1, Y2, Y3, Y4, Y5 and Y6 is independently selected from H, F, Cl, CHF₂, and CF₃; 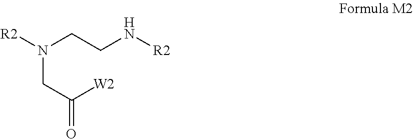
wherein W2 is absent and represents the point of attachment * to said human insulin or human insulin analogue, or W2 represents the D- or L-form of NH—CH(COOH)—CH₂CH₂—C(═O)—*, or NH—CH₂CH₂CH₂—C(═O)—*, wherein * represents the point of attachment to said human insulin or human insulin analogue; and
wherein R2 is selected from 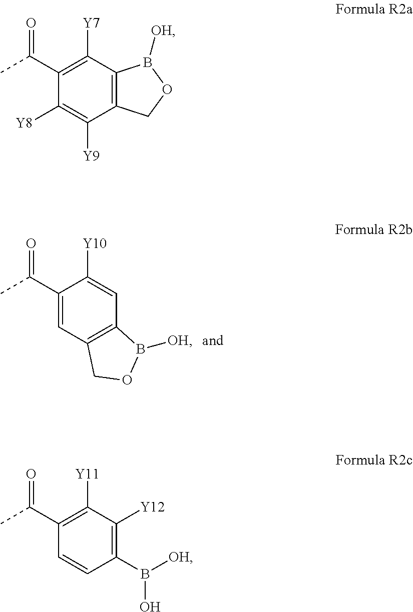
wherein Y7, Y8, Y9, Y10, Y1l and Y12 is independently selected from H, F, Cl, CHF₂, and CF₃; 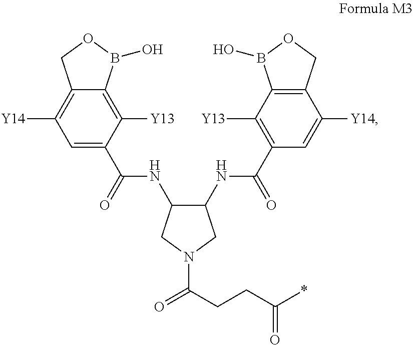
which represents a R,R or S,S, or R,S stereoisomer of the 3,4-diamino-pyrrolidine; and wherein * represents the point of attachment to said human insulin or human insulin analogue; and wherein Y13 and Y14 is independently selected from H, F, Cl, CHF₂, and CF₃; 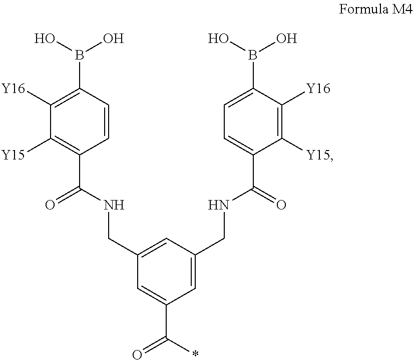
wherein * represents the point of attachment to said human insulin or human insulin analogue, and
wherein Y15 and Y16 is independently selected from H, F, Cl, CHF₂, and CF₃; 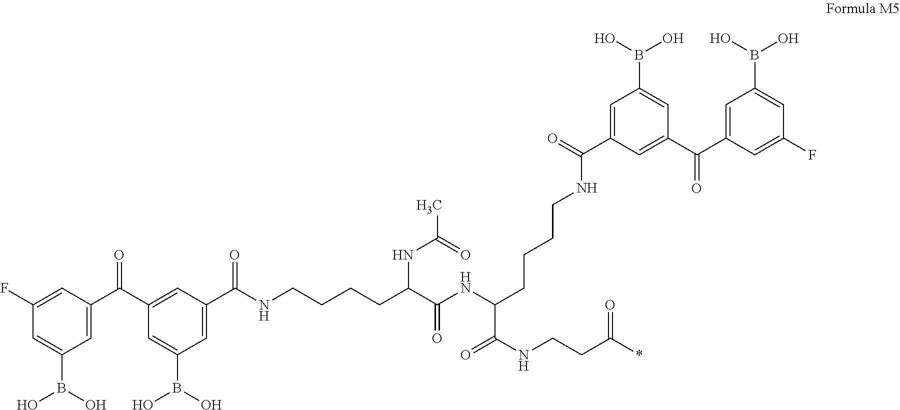 [SMILES: *C(=O)CCNC(=O)C(CCCCNC(=O)c1cc(B(O)O)cc(C(=O)c2cc(F)cc(B(O)O)c2)c1)NC(=O)C(CCCCNC(=O)c1cc(B(O)O)cc(C(=O)c2cc(F)cc(B(O)O)c2)c1)NC(C)=O]
wherein each of the amino acid residues represents a D- or an L-amino acid form, and wherein * represents the point of attachment to said human insulin or human insulin analogue; 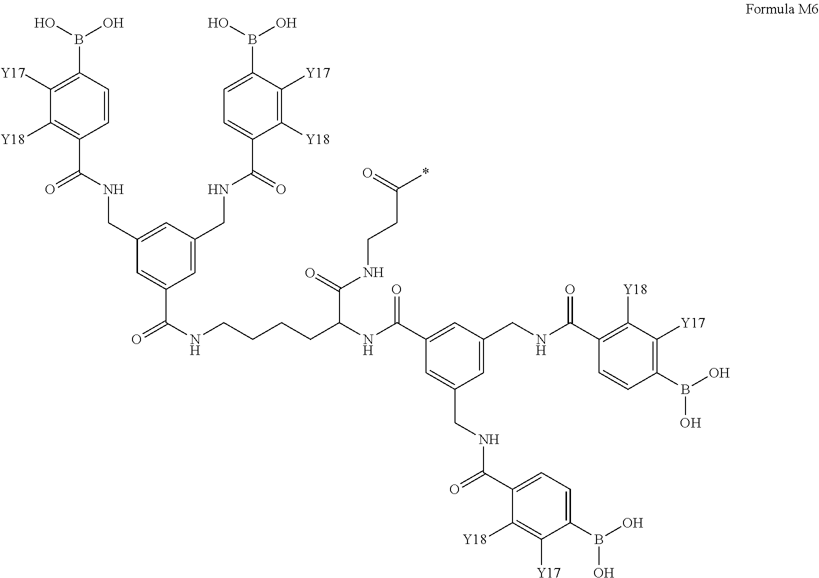
wherein the □-amino acid residue represents a D- or an L-amino acid form, and wherein * represents the point of attachment to said human insulin or human insulin analogue; and wherein Y17 and Y18 is independently selected from H, F, Cl, CHF₂, and CF₃; 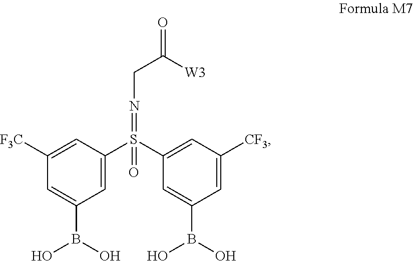
wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue; 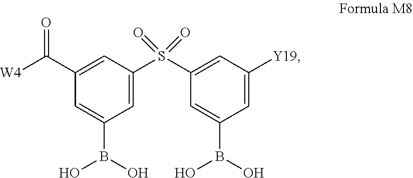
wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is H, F, Cl, CHF₂, and CF₃ or SF₅; 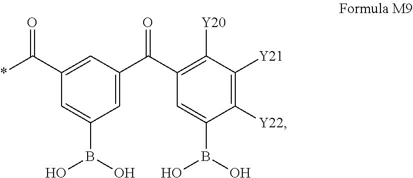
wherein * represents the point of attachment to said human insulin or human insulin analogue; and
wherein each of Y20, Y21, and Y22 is independently selected from H, F, Cl, CHF₂, and CF₃; 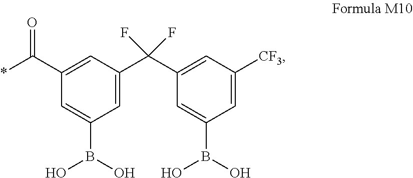 [SMILES: *C(=O)c1cc(B(O)O)cc(C(F)(F)c2cc(B(O)O)cc(C(F)(F)F)c2)c1]
wherein * represents the point of attachment to said human insulin or human insulin analogue; and 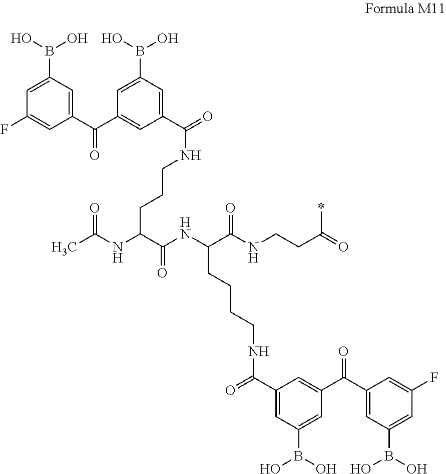 [SMILES: *C(=O)CCNC(=O)C(CCCCNC(=O)c1cc(B(O)O)cc(C(=O)c2cc(F)cc(B(O)O)c2)c1)NC(=O)C(CCCNC(=O)c1cc(B(O)O)cc(C(=O)c2cc(F)cc(B(O)O)c2)c1)NC(C)=O]
wherein each of the amino acid residues represents a D- or an L-amino acid form, and wherein * represents the point of attachment to said human insulin or human insulin analogue.

2. The compound according to claim 1, wherein each of the modifying groups M is independently selected from the group of 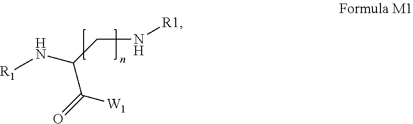
which represents a D- or an L-amino acid form, and wherein n is 1;
W1 represents NH—CH₂CH₂—C(═O)—* or the D- or L-form of NH—CH(COOH)—CH₂CH₂—C(═O)—*,
wherein * represents the point of attachment to said human insulin or human insulin analogue; and
R1 is of 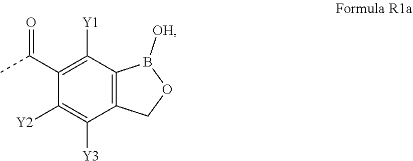
wherein Y1 and Y2 are H; and Y3 is F or CF₃; 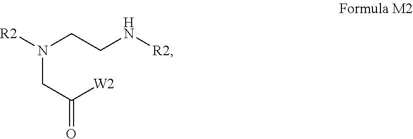
wherein W2 is absent and represents the point of attachment * to said human insulin or human insulin analogue, or W2 represents the D- or L-form of NH—CH(COOH)—CH₂CH₂—C(═O)—*, wherein * represents the point of attachment to said human insulin or human insulin analogue; and
wherein R2 is of 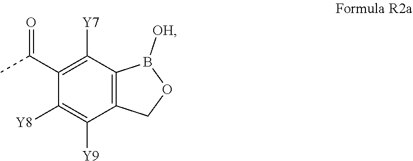
wherein Y7 and Y8 are H; and Y9 is Cl, CHF₂, or CF₃; 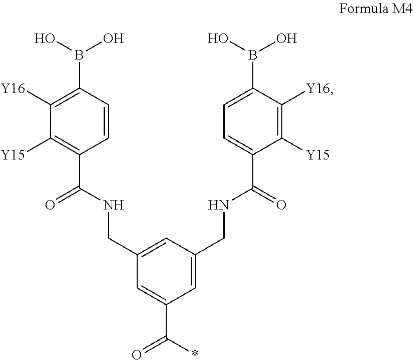
wherein * represents the point of attachment to said human insulin or human insulin analogue, and
wherein Y15 is H, and Y16 is F; 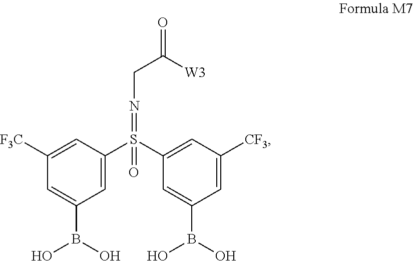
wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue; 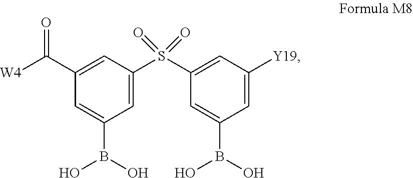
wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is CF₃; and 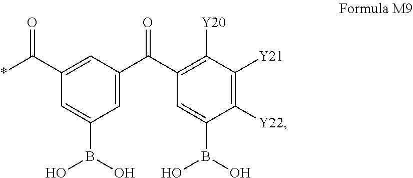
wherein * represents the point of attachment to said human insulin or human insulin analogue; and
wherein each of Y20, Y21, and Y22 is independently selected from H and F; with the provisio that when Y21 is F, then Y20 and Y22 are H; and when Y21 is H, then Y20 and Y22 are F.

3. The compound according to claim 1, wherein
said human insulin or human insulin analogue optionally comprises a spacer selected from the group of
a) a peptide spacer at the C-terminal of the A-chain of said human insulin or human insulin analogue,
wherein said peptide spacer comprises (GES)ₚK, wherein p is an integer from 3 to 12; or
b) a peptide spacer or a linker L at the N-terminal of the B-chain of said human insulin or human insulin analogue;
wherein said peptide spacer comprises GKPG, GKP(G4S)_{q}, KP(G4S)ᵣ, GKPRGFFYTP(G4S)ₛ, or TYFFGRKPD(G4S)ₜ, wherein each of q, r, s and t is independently selected from an integer from 1 to 5; and
wherein said linker L is selected from 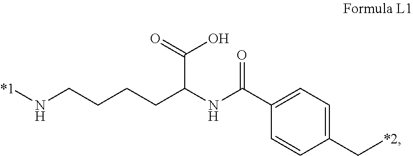
wherein *1 denotes the attachment point to the modifying group M and *2 denotes the attachment point to the amino group of the amino acid residue at the N-terminal of the B-chain of said human insulin or human insulin analogue; 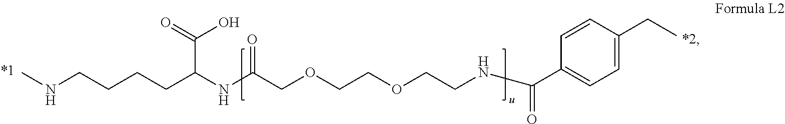
wherein *1 denotes the attachment point to the modifying group M and *2 denotes the attachment point to the amino group of the amino acid residue at N-terminal of the B-chain of said human insulin or human insulin analogue, and
wherein u is 1, 2 or 3; and 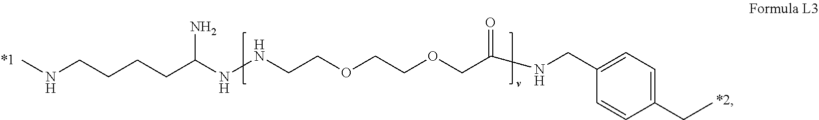
wherein *1 denotes the attachment point to the modifying group M and *2 denotes the attachment point to the amino group of the amino acid residue at N-terminal of the B-chain of said human insulin or human insulin analogue, and
wherein v is 2 or 3.

4. The compound according to claim 3, wherein q is an integer selected from 1 to 3; r is 3; s is 2; and t is 3.

5. The compound according to claim 1, wherein each modifying group M is attached to an attachment point selected from one of the following groups:
a) the amino group of the N-terminal amino acid residue of the A-chain of said human insulin or human insulin analogue;
b) the epsilon amino group of a lysine in position 22 of the A-chain of said human insulin analogue; or
the epsilon amino group of the lysine in said optional peptide spacer at the C-terminal of the A-chain of said human insulin or human insulin analogue;
c) the amino group of the N-terminal amino acid residue of the B-chain of said human insulin or human insulin analogue;
the epsilon amino group of a lysine residue in position 1 or position 4 of the B-chain of said human insulin analogue;
the epsilon amino group of a lysine in said optional peptide spacer at the N-terminal of the B-chain of said human insulin or human insulin analogue; or
the distal amino group marked with *1 in said optional linker L at the N-terminal of the B-chain of said human insulin or human insulin analogue; and
d) the epsilon amino group of a lysine in position 22 or position 29 of the B-chain of said human insulin or human insulin analogue.

6. The compound according to claim 1, having exactly two, three or four modifying groups M.

7. The compound according to claim 1, wherein said human insulin or human insulin analogue is a human insulin analogue selected from the group of
desB30 human insulin;
A21Q desB30 human insulin;
A14E B25H desB30 human insulin;
A14E B1K B2P B25H desB27 desB30 human insulin;
A14E A22K B25H desB27 desB30 human insulin;
A14E A22K B25H B27P B28G desB30 human insulin;
A14E desB1-B2 B4K B5P desB30 human insulin;
A14E desB1-B2 B3G B4K B5P desB30 human insulin;
A14E B-1G B1K B2P desB30 human insulin;
A22K desB30 human insulin;
A22K B29R desB30 human insulin;
A22K B22K B29R desB30 human insulin; and
A-2K A-1P desB30 human insulin.

8. A compound according to claim 1, comprising
i) human insulin or a human insulin analogue, wherein said human insulin or human insulin analogue optionally comprises a peptide spacer at the N-terminal of the B-chain of said human insulin or human insulin analogue;
wherein said peptide spacer comprises GKPG, GKP(G4S)_{q}, KP(G4S)ᵣ, GKPRGFFYTP(G4S)ₛ, or TYFFGRKPD(G4S)ₜ, wherein q is an integer from 1 to 3; r is 3; s is 2 and t is 3;
ii) two modifying groups M, wherein each of the modifying groups M is independently selected from the group of 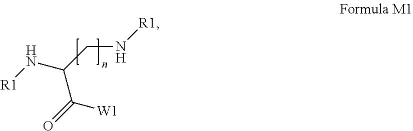
which represents a D- or an L-amino acid form, and wherein n is 1; W1 represents NH—CH₂CH₂—C(═O)—*, or the D- or L-form of NH—CH(COOH)—CH₂CH₂—C(═O)—*, wherein * represents the point of attachment to said human insulin or human insulin analogue; and
wherein R1 is of 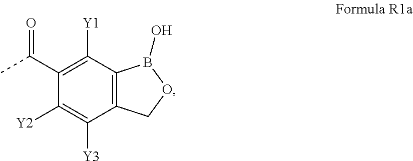
wherein Y1 and Y2 is H, and Y3 is CF₃; 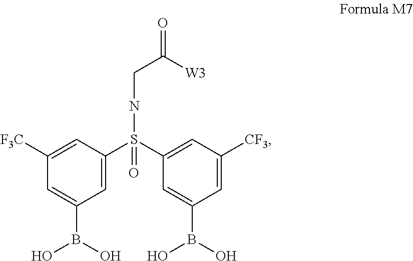
wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue; 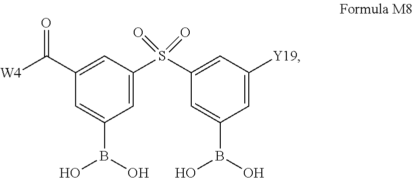
wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is CF₃; 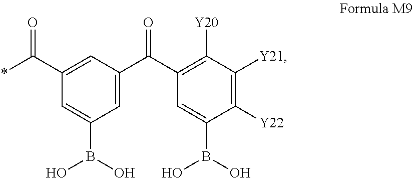
wherein * represents the point of attachment to said human insulin or human insulin analogue; and
wherein each of Y20, Y21, and Y22 is independently selected from H, and F; with the provisio that when Y21 is F, then Y20 and Y22 are H; and when Y21 is H, then Y20 and Y22 are F; and
wherein one modifying group M is attached to the epsilon amino group of a lysine in position 29 of the B-chain of said human insulin or human insulin analogue; and one modifying group M is attached to
the epsilon amino group of a lysine residue in position 1 or position 4 of the B-chain of said human insulin analogue; or
the epsilon amino group of a lysine in said optional peptide spacer at the N-terminal of the B-chain of said human insulin or human insulin analogue.

9. The compound according to claim 1, wherein the compound is selected from the group of:

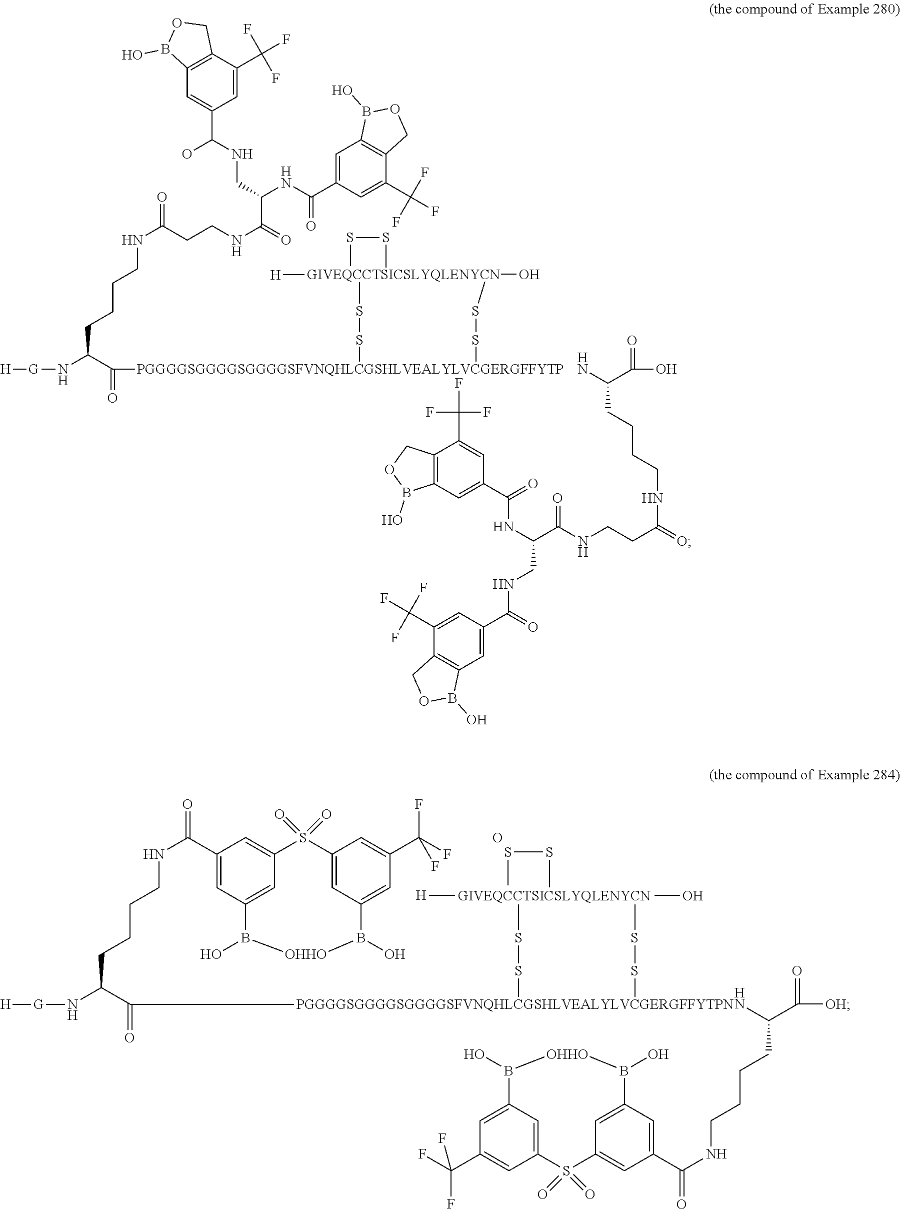

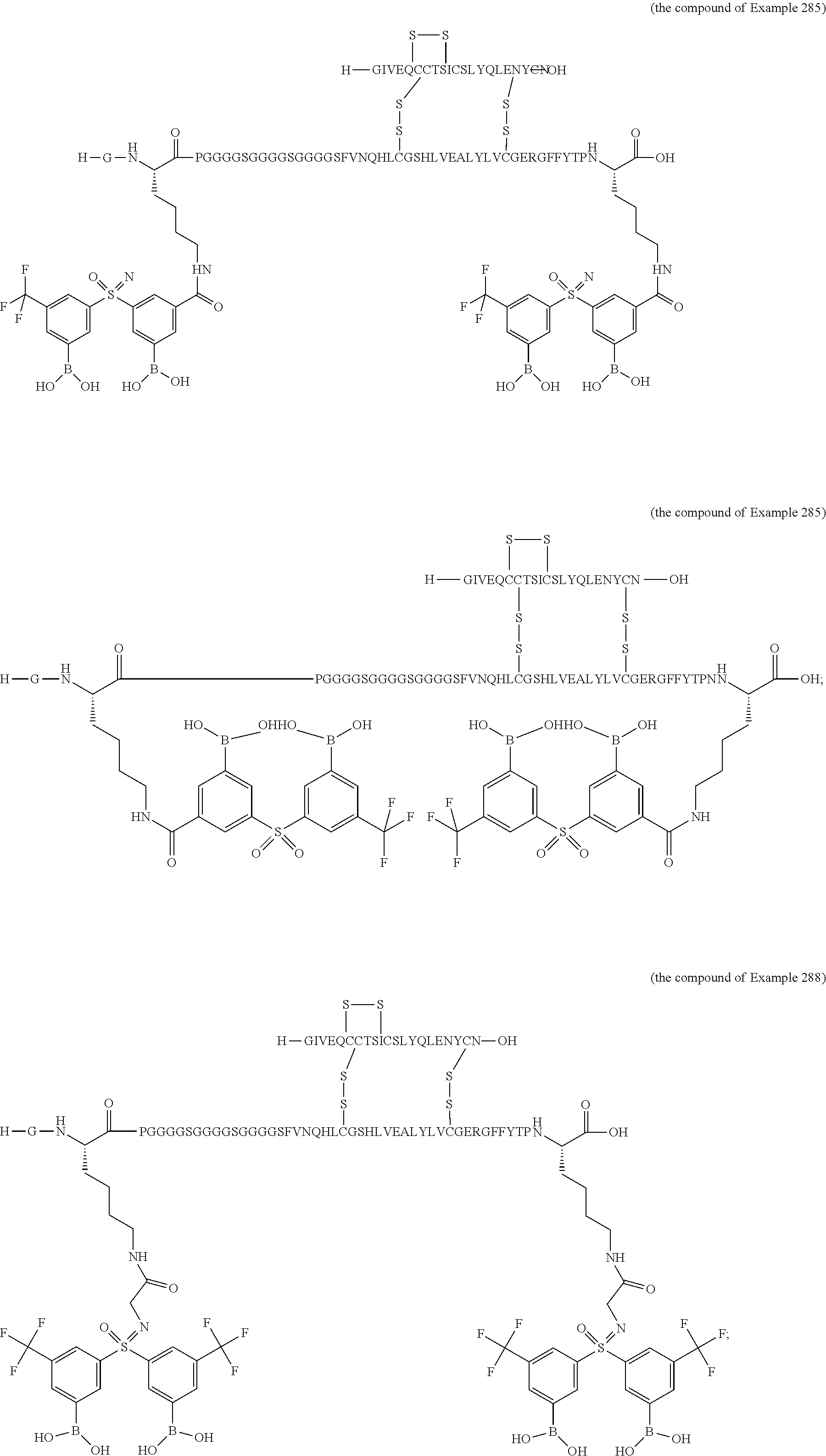

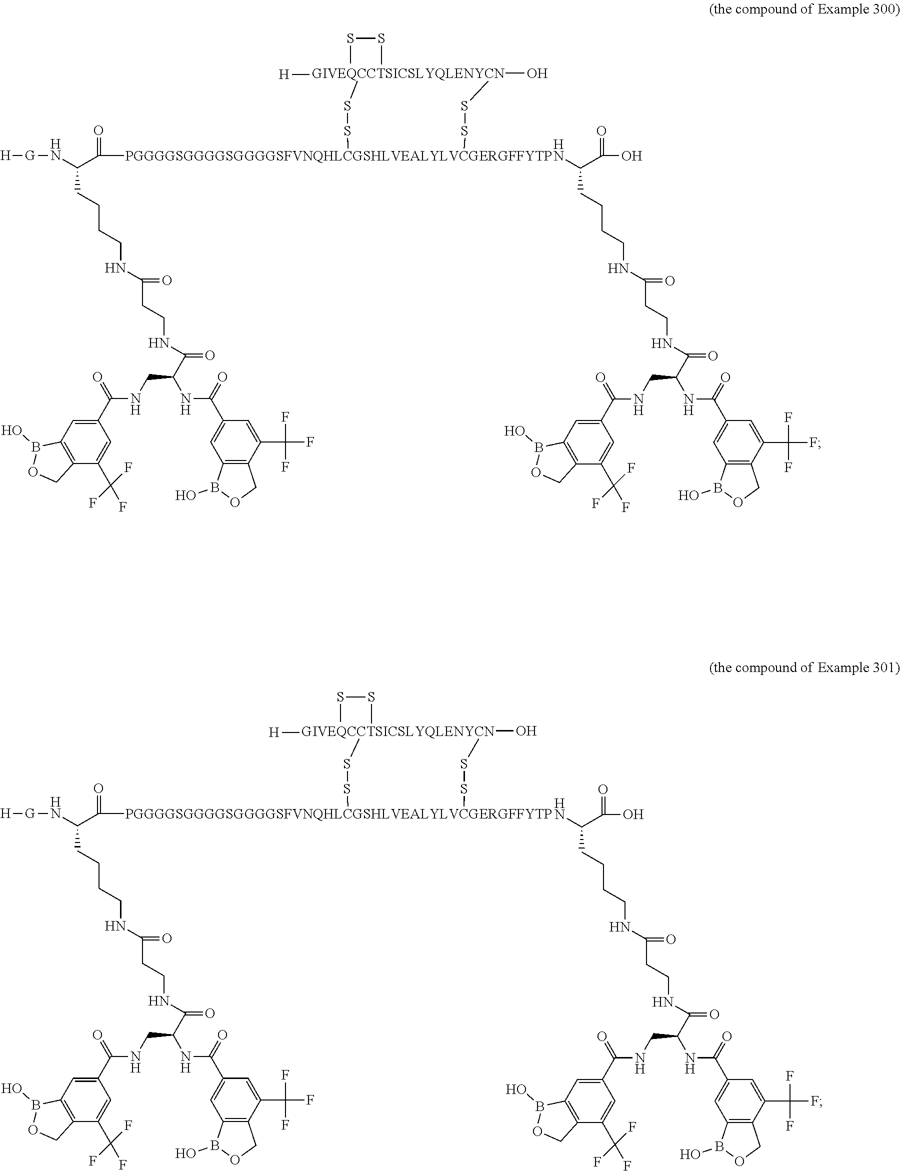

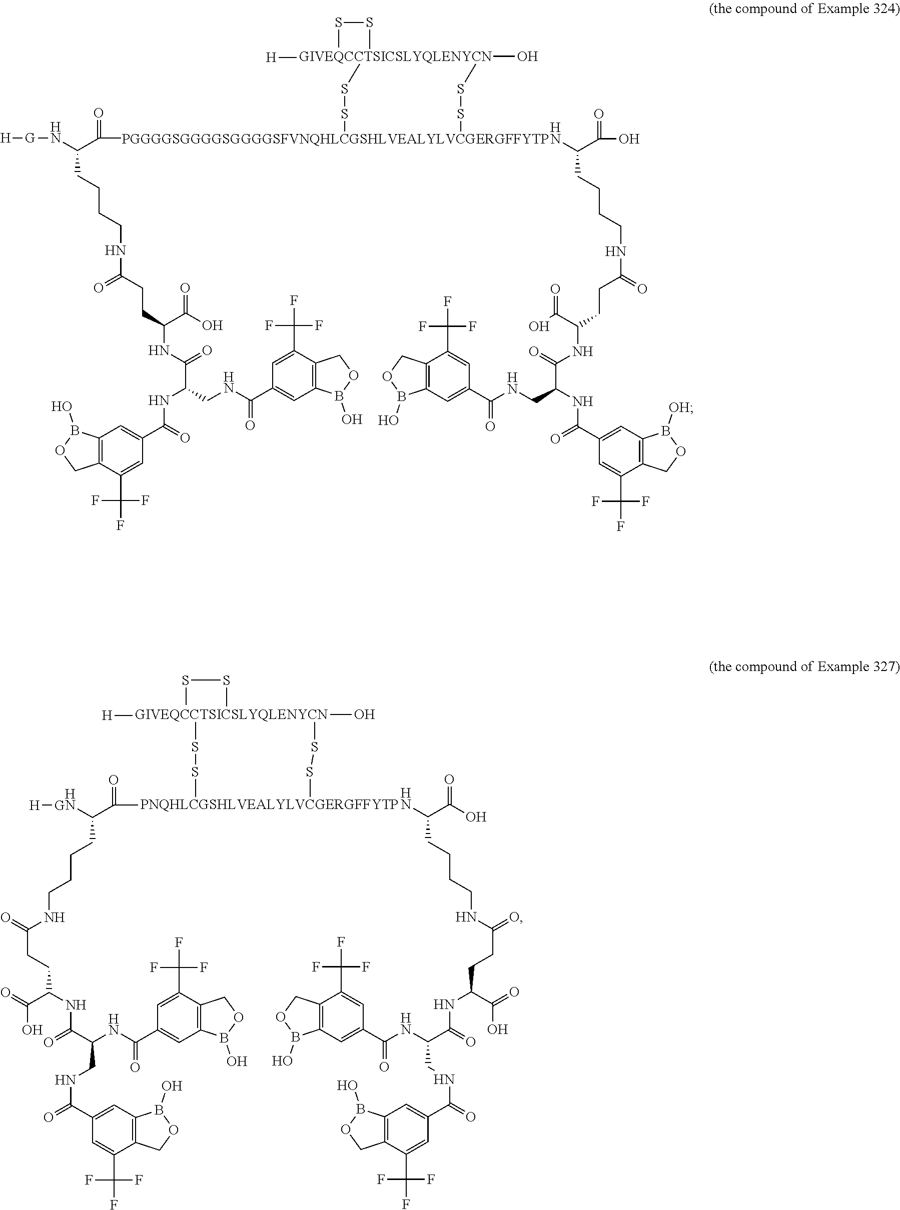

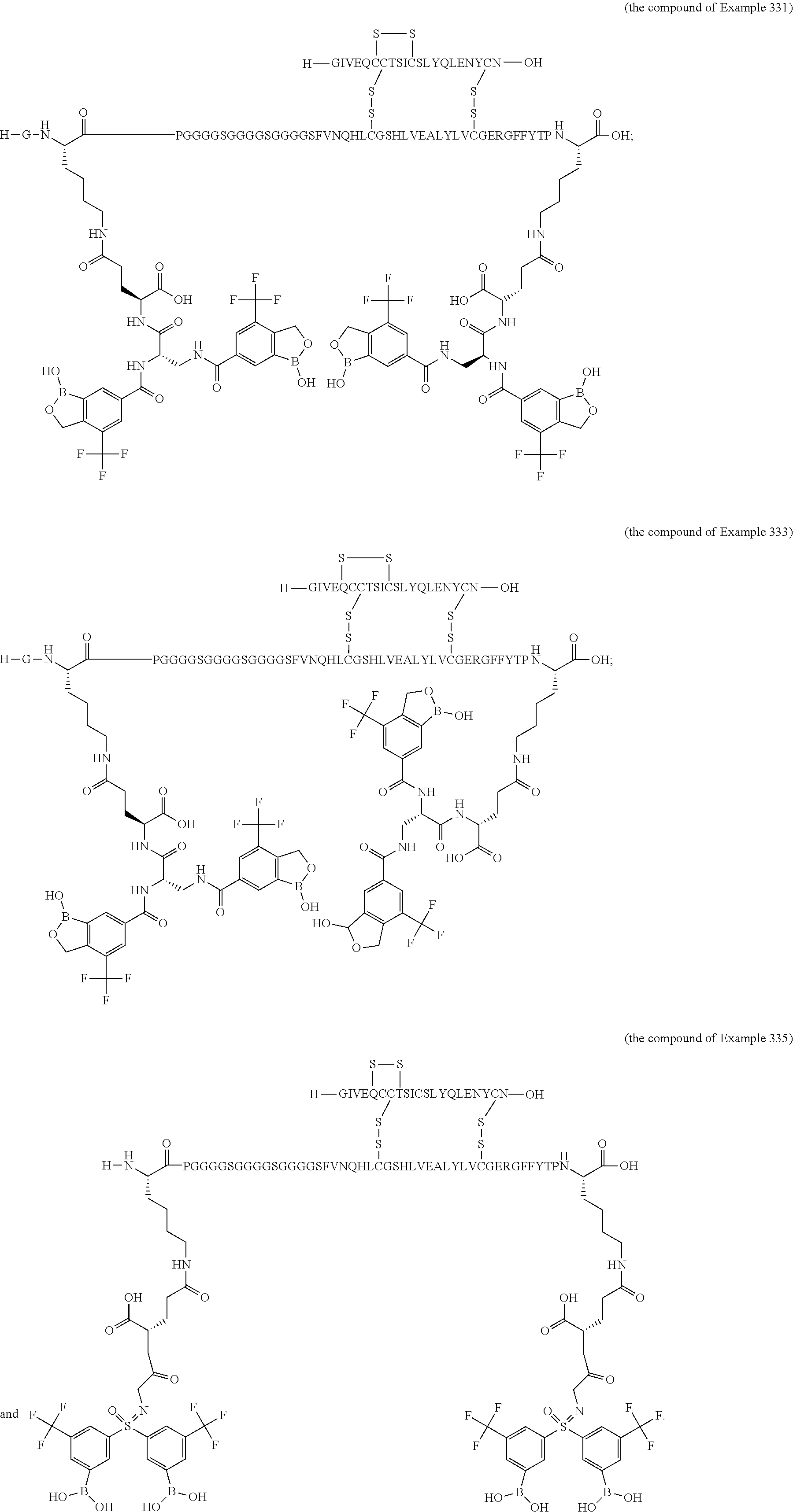

10. An intermediate product selected from the group consisting of
A14E desB1-B2 B4K B5P desB30 human insulin (SEQ ID NO:4 and SEQ ID NO:16);
A14E desB1-B2 B3G B4K B5P desB30 human insulin (SEQ ID NO:4 and SEQ ID NO:17);
A14E B-1G B1K B2P desB30 human insulin (SEQ ID NO:4 and SEQ ID NO:18);
A22K B22K B29R desB30 human insulin (SEQ ID NO:6 and SEQ ID NO:20);
A21Q (GES)3K desB30 human insulin (SEQ ID NO:8 and SEQ ID NO:11);
A21Q (GES)6K desB30 human insulin (SEQ ID NO:9 and SEQ ID NO:11);
A21Q (GES)12K desB30 human insulin (SEQ ID NO:10 and SEQ ID NO:11);
B1-KPGGGGSGGGGSGGGGS desB30 human insulin (SEQ ID NO:1 and SEQ ID NO:21);
B1-KPGGGGSGGGGSGGGGS A14E B25H desB30 human insulin (SEQ ID NO:4 and SEQ ID NO:22);
B1-GKPGGGGSGGGGSGGGGS desB30 human insulin (SEQ ID NO:1 and SEQ ID NO:23);
B1-GKPGGGGSGGGGS desB30 human insulin (SEQ ID NO:1 and SEQ ID NO:24);
B1-GKPGGGGS desB30 human insulin (SEQ ID NO:1 and SEQ ID NO:25);
B1-GKPG desB30 human insulin (SEQ ID NO:1 and SEQ ID NO:26);
B1-GKPRGFFYTPGGGGSGGGGS desB30 human insulin (SEQ ID NO:1 and SEQ ID NO:27); and
B1-TYFFGRKPDGGGGSGGGGSGGGGS desB30 human insulin (SEQ ID NO:1 and SEQ ID NO:28).

11. A composition comprising a compound according to claim 1.

12. (canceled)

13. (canceled)

14. (canceled)

15. The compound according to claim 2, wherein said human insulin or human insulin analogue optionally comprises a spacer selected from the group of
a) a peptide spacer at the C-terminal of the A-chain of said human insulin or human insulin analogue,
wherein said peptide spacer comprises (GES)ₚK, wherein p is an integer from 3 to 12; or
b) a peptide spacer or a linker L at the N-terminal of the B-chain of said human insulin or human insulin analogue;
wherein said peptide spacer comprises GKPG, GKP(G4S)_{q}, KP(G4S)ᵣ, GKPRGFFYTP(G4S)ₛ, or TYFFGRKPD(G4S)ₜ, wherein each of q, r, s and t is independently selected from an integer from 1 to 5; and
wherein said linker L is selected from 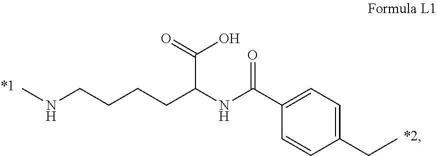
wherein *1 denotes the attachment point to the modifying group M and *2 denotes the attachment point to the amino group of the amino acid residue at the N-terminal of the B-chain of said human insulin or human insulin analogue; 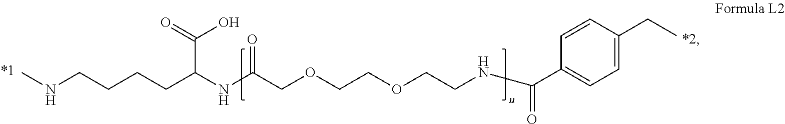
wherein *1 denotes the attachment point to the modifying group M and *2 denotes the attachment point to the amino group of the amino acid residue at N-terminal of the B-chain of said human insulin or human insulin analogue, and
wherein u is 1, 2 or 3; and 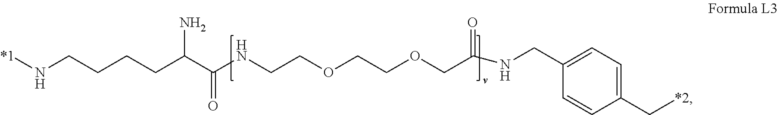
wherein *1 denotes the attachment point to the modifying group M and *2 denotes the attachment point to the amino group of the amino acid residue at N-terminal of the B-chain of said human insulin or human insulin analogue, and
wherein v is 2 or 3.

16. The compound according to claim 15, wherein q is an integer selected from 1 to 3; r is 3; s is 2; and t is 3.

17. The compound according to claim 2, wherein each modifying group M is attached to an attachment point selected from one of the following groups:
a) the amino group of the N-terminal amino acid residue of the A-chain of said human insulin or human insulin analogue;
b) the epsilon amino group of a lysine in position 22 of the A-chain of said human insulin analogue; or
the epsilon amino group of the lysine in said optional peptide spacer at the C-terminal of the A-chain of said human insulin or human insulin analogue;
c) the amino group of the N-terminal amino acid residue of the B-chain of said human insulin or human insulin analogue;
the epsilon amino group of a lysine residue in position 1 or position 4 of the B-chain of said human insulin analogue;
the epsilon amino group of a lysine in said optional peptide spacer at the N-terminal of the B-chain of said human insulin or human insulin analogue; or
the distal amino group marked with *1 in said optional linker L at the N-terminal of the B-chain of said human insulin or human insulin analogue; and
d) the epsilon amino group of a lysine in position 22 or position 29 of the B-chain of said human insulin or human insulin analogue.

18. The compound according to claim 2, having exactly two, three or four modifying groups M.

19. The compound according to claim 2, wherein said human insulin or human insulin analogue is a human insulin analogue selected from the group of
desB30 human insulin;
A21Q desB30 human insulin;
A14E B25H desB30 human insulin;
A14E B1K B2P B25H desB27 desB30 human insulin;
A14E A22K B25H desB27 desB30 human insulin;
A14E A22K B25H B27P B28G desB30 human insulin;
A14E desB1-B2 B4K B5P desB30 human insulin;
A14E desB1-B2 B3G B4K B5P desB30 human insulin;
A14E B-1G B1K B2P desB30 human insulin;
A22K desB30 human insulin;
A22K B29R desB30 human insulin;
A22K B22K B29R desB30 human insulin; and
A-2K A-1P desB30 human insulin.

20. A compound according to claim 2, comprising
i) human insulin or a human insulin analogue, wherein said human insulin or human insulin analogue optionally comprises a peptide spacer at the N-terminal of the B-chain of said human insulin or human insulin analogue;
wherein said peptide spacer comprises GKPG, GKP(G4S)_{q}, KP(G4S)ᵣ, GKPRGFFYTP(G4S)ₛ, or TYFFGRKPD(G4S)ₜ, wherein q is an integer from 1 to 3; r is 3; s is 2 and t is 3;
ii) two modifying groups M, wherein each of the modifying groups M is independently selected from the group of 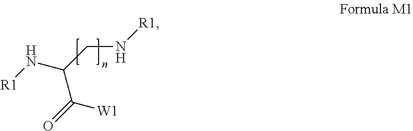
which represents a D- or an L-amino acid form, and wherein n is 1; W1 represents NH—CH₂CH₂—C(═O)—*, or the D- or L-form of NH—CH(COOH)—CH₂CH₂—C(═O)—*, wherein * represents the point of attachment to said human insulin or human insulin analogue; and
wherein R1 is of 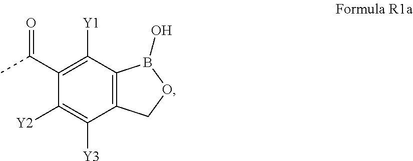
wherein Y1 and Y2 is H, and Y3 is CF₃; 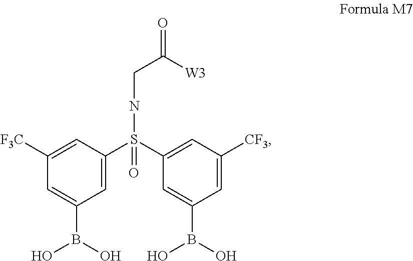
wherein W3 is absent and represents the point of attachment * to said human insulin or human insulin analogue, or W3 represents the D- or L-form of NH—CH(COOH)—CH₂CH₂—C(═O)—*, wherein * represents the point of attachment to said human insulin or human insulin analogue; 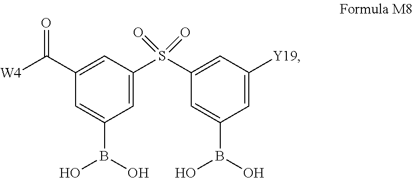
wherein W4 is absent and represents the point of attachment * to said human insulin or human insulin analogue, or W4 represents NH—CH₂—C(═O)—*, wherein * represents the point of attachment to said human insulin or human insulin analogue; and wherein Y19 is CF₃; 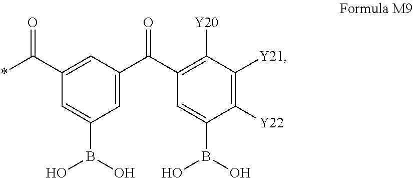
wherein * represents the point of attachment to said human insulin or human insulin analogue; and
wherein each of Y20, Y21, and Y22 is independently selected from H, and F; with the provisio that when Y21 is F, then Y20 and Y22 are H; and when Y21 is H, then Y20 and Y22 are F; and
wherein one modifying group M is attached to the epsilon amino group of a lysine in position 29 of the B-chain of said human insulin or human insulin analogue; and one modifying group M is attached to
the epsilon amino group of a lysine residue in position 1 or position 4 of the B-chain of said human insulin analogue; or
the epsilon amino group of a lysine in said optional peptide spacer at the N-terminal of the B-chain of said human insulin or human insulin analogue.

21. A method for treating diabetes, diabetes of Type 1, diabetes of Type 2, impaired glucose tolerance, hyperglycemia, metabolic syndrome X or insulin resistance syndrome, comprising administration to a subject in need thereof a therapeutically effective amount of a compound according to claim 1.

22. A method for treating diabetes, diabetes of Type 1, diabetes of Type 2, impaired glucose tolerance, hyperglycemia, metabolic syndrome X or insulin resistance syndrome, comprising administration to a subject in need thereof a therapeutically effective amount of a compound according to claim 9.
